SEQUENCE LISTING (190 sequences)

SEQ ID NO: 1 - DNA, 21 bp, synthetic construct
  misc_feature 1..21: note = Description of Artificial Sequence: Synthetic oligonucleotide
  source 1..21: mol_type = other DNA
agacatgattacattaattaa

SEQ ID NO: 2 - DNA, 21 bp, synthetic construct
  misc_feature 1..21: note = Description of Artificial Sequence: Synthetic oligonucleotide
  source 1..21: mol_type = other DNA
cctcctgtttctgaatacaaa

SEQ ID NO: 3 - DNA, 21 bp, synthetic construct
  misc_feature 1..21: note = Description of Artificial Sequence: Synthetic oligonucleotide
  source 1..21: mol_type = other DNA
tcctgggaactatctaattaa

SEQ ID NO: 4 - DNA, 21 bp, synthetic construct
  misc_feature 1..21: note = Description of Artificial Sequence: Synthetic oligonucleotide
  source 1..21: mol_type = other DNA
agtgaaaattctacaatcata

SEQ ID NO: 5 - DNA, 21 bp, synthetic construct
  misc_feature 1..21: note = Description of Artificial Sequence: Synthetic oligonucleotide
  source 1..21: mol_type = other DNA
tgatattcacagattatgtta

SEQ ID NO: 6 - DNA, 21 bp, synthetic construct
  misc_feature 1..21: note = Description of Artificial Sequence: Synthetic oligonucleotide
  source 1..21: mol_type = other DNA
ccctcctgtttctgaatacaa

SEQ ID NO: 7 - DNA, 21 bp, synthetic construct
  misc_feature 1..21: note = Description of Artificial Sequence: Synthetic oligonucleotide
  source 1..21: mol_type = other DNA
cagacatgattacattaatta

SEQ ID NO: 8 - DNA, 21 bp, synthetic construct
  misc_feature 1..21: note = Description of Artificial Sequence: Synthetic oligonucleotide
  source 1..21: mol_type = other DNA
tccctgattggtatttagaaa

SEQ ID NO: 9 - DNA, 21 bp, synthetic construct
  misc_feature 1..21: note = Description of Artificial Sequence: Synthetic oligonucleotide
  source 1..21: mol_type = other DNA
gatattcacagattatgttaa

SEQ ID NO: 10 - DNA, 21 bp, synthetic construct
  misc_feature 1..21: note = Description of Artificial Sequence: Synthetic oligonucleotide
  source 1..21: mol_type = other DNA
gacagtgaactgtttacagta

SEQ ID NO: 11 - DNA, 21 bp, synthetic construct
  misc_feature 1..21: note = Description of Artificial Sequence: Synthetic oligonucleotide
  source 1..21: mol_type = other DNA
gggaactatctaattaacgta

SEQ ID NO: 12 - DNA, 21 bp, synthetic construct
  misc_feature 1..21: note = Description of Artificial Sequence: Synthetic oligonucleotide
  source 1..21: mol_type = other DNA
tggcaactgtttgaatagaaa

SEQ ID NO: 13 - DNA, 21 bp, synthetic construct
  misc_feature 1..21: note = Description of Artificial Sequence: Synthetic oligonucleotide
  source 1..21: mol_type = other DNA
aactgtttgaatagaaattta

SEQ ID NO: 14 - DNA, 21 bp, synthetic construct
  misc_feature 1..21: note = Description of Artificial Sequence: Synthetic oligonucleotide
  source 1..21: mol_type = other DNA
cccggctaagtttttaatttt

SEQ ID NO: 15 - DNA, 21 bp, synthetic construct
  misc_feature 1..21: note = Description of Artificial Sequence: Synthetic oligonucleotide
  source 1..21: mol_type = other DNA
ccatacatgcagacatgatta

SEQ ID NO: 16 - DNA, 21 bp, synthetic construct
  misc_feature 1..21: note = Description of Artificial Sequence: Synthetic oligonucleotide
  source 1..21: mol_type = other DNA
ccaaacaaaatattttatcaa

SEQ ID NO: 17 - DNA, 21 bp, synthetic construct
  misc_feature 1..21: note = Description of Artificial Sequence: Synthetic oligonucleotide
  source 1..21: mol_type = other DNA
accgtatttcaagtattctga

SEQ ID NO: 18 - DNA, 21 bp, synthetic construct
  misc_feature 1..21: note = Description of Artificial Sequence: Synthetic oligonucleotide
  source 1..21: mol_type = other DNA
tctgagaaaaatcatatctta

SEQ ID NO: 19 - DNA, 21 bp, synthetic construct
  misc_feature 1..21: note = Description of Artificial Sequence: Synthetic oligonucleotide
  source 1..21: mol_type = other DNA
cacagattatgttaaaagttt

SEQ ID NO: 20 - DNA, 21 bp, synthetic construct
  misc_feature 1..21: note = Description of Artificial Sequence: Synthetic oligonucleotide
  source 1..21: mol_type = other DNA
ccactgctattgtagtgaaaa

SEQ ID NO: 21 - DNA, 1446 bp, synthetic construct
  misc_feature 1..1446: note = Description of Artificial Sequence: Synthetic polynucleotide
  source 1..1446: mol_type = other DNA
atgagcacactgtgtcctccaccgagccctgccgtggccaagacagagatcgccctgagcggcaagtcccctctgctggccgccacattcgcctactgggacaacatcctgggacctagagttagacacatttgggcccctaagaccgagcaggtgctgctgagtgatggagagatcaccttcctggccaaccacaccctgaacggcgagatcctgagaaatgccgagagcggcgctatcgatgtgaagttcttcgtgctgtctgagaagggtgttatcatcgtgtccctgatcttcgacggcaactggaacggcgatagaagcacctacggcctgagcatcatcctgcctcagaccgagctgagcttctacctgccactgcacagagtgtgcgtggacagactgacacacatcattagaaagggaagaatctggatgcacaaggaaagacaggagaacgtgcaaaagatcatcctggaaggtacagagcggatggaagatcagggccagagcatcatacccatgctgacaggcgaagtgatccccgtgatggaactcctcagctccatgaaaagccacagcgtgcccgaggaaatcgacatcgccgacaccgtgctgaatgacgacgacatcggcgacagctgccacgaaggcttcctgctgaacgccatcagcagccacctgcagacatgcggctgcagcgtcgtggtgggctcttctgccgagaaggtgaacaagatcgtgcggaccctgtgcctgttcctgacacctgctgagaggaagtgcagcagactgtgtgaagccgaatccagctttaagtacgagtctggcctgtttgtgcaaggcctcctgaaagactccaccggcagctttgtgctgccttttagacaggtgatgtacgccccttaccccaccacccacatcgacgtcgacgtgaacaccgtgaagcagatgcctccgtgccacgagcacatctacaaccagcggagatacatgagaagcgagctgaccgccttctggcgggccaccagcgaggaagatatggcacaggacaccatcatctacaccgacgagagcttcacccctgacctgaacatcttccaagatgtgctgcaccgggacaccctggtgaaagccttcctggatcaggtctttcagctgaaacccggcctgtctctgagatctaccttcctggcccagttcctgcttgtgctgcatagaaaggccctgacgctgatcaagtacatcgaggatgatacacagaaaggaaaaaagcccttcaagagcctgcggaacctgaagatcgacctggacctgactgccgagggcgacctgaacatcatcatggccctggctgaaaagattaagccaggcctgcactccttcatctttggcagacctttctacacctccgtgcaggagagagatgtgctgatgaccttctga

SEQ ID NO: 22 - DNA, 1446 bp, synthetic construct
  misc_feature 1..1446: note = Description of Artificial Sequence: Synthetic polynucleotide
  source 1..1446: mol_type = other DNA
atgagcacactctgtcctccccccagccccgccgtggccaagaccgagatcgccctgagcggaaagtcccctctgcttgctgctacatttgcctactgggacaacatcttgggccctagagtgcggcacatctgggcccctaagaccgagcaggtcctgctgagtgatggcgaaatcaccttcctggctaatcacaccctgaacggcgagatcctgagaaacgccgagtccggcgccatcgatgtgaagttcttcgtgctgtctgaaaagggcgtgatcattgtgtccctgatcttcgacggaaattggaacggcgatagatctacctacggcctgtctatcatcctgcctcagacagagctgagcttctacctgcccctgcacagagtgtgcgtggaccggctgacacacattatcagaaagggcagaatctggatgcacaaggaacgccaggagaacgtgcagaagatcatcctggaaggcaccgagagaatggaagatcagggccagagcatcatccccatgctgaccggcgaggtgattcctgtgatggaactgctgagcagcatgaaaagccactccgtccccgaggaaatcgacatcgcagataccgtgctgaacgacgatgacatcggcgacagctgccacgagggattcctcctgaatgccatcagctctcacctgcagacatgcggctgtagcgtcgtcgtgggcagcagcgccgagaaagtgaacaagatcgtgcggacactgtgtctgttcctcacacctgccgaaagaaagtgcagcagactgtgcgaggccgagtctagcttcaagtacgagagcggcctcttcgtgcagggactgctgaaggacagcaccggctctttcgtgctgcctttcagacaggtgatgtacgccccttaccccaccacccacatcgacgttgacgtgaacaccgtgaaacagatgcccccgtgccatgaacacatctacaaccagcggagatacatgagaagcgagctgaccgccttctggcgggccaccagcgaggaagatatggctcaggataccatcatctatacagacgagagcttcacccctgacctgaacatctttcaggacgtgctgcatagagatacactcgtgaaggcctttctggatcaggttttccagctgaagcctggcctgagcctgagatccaccttcctggcacaatttctgctggtgctgcaccggaaggccctgaccctgatcaagtacatcgaggacgacacacagaaaggcaagaagccctttaagagcctgcggaacctgaaaattgatctggacctgactgccgagggcgacctgaatatcatcatggccctggccgagaagatcaagcctggactgcactctttcatcttcggcagacctttctacacaagcgtgcaagagcgggacgtgctgatgaccttctga

SEQ ID NO: 23 - DNA, 1446 bp, synthetic construct
  misc_feature 1..1446: note = Description of Artificial Sequence: Synthetic polynucleotide
  source 1..1446: mol_type = other DNA
atgagcaccctgtgtcctccgcccagccctgccgtggccaagaccgaaatcgccctgagcggaaaaagccccctgctggccgccacctttgcctactgggacaacatcctgggccctagagtgcggcacatctgggcccctaagaccgagcaggtgctgctgagcgacggcgagataacattcctcgctaatcacacactgaacggcgaaatcctgagaaatgccgaaagcggcgccatcgacgttaagttcttcgtgctgtctgaaaagggcgtgatcatcgtgtccctgatcttcgacggcaactggaacggcgatagatcaacctacggcctgagcatcatcctgcctcagaccgagctgtctttctacctgcctctgcatagagtgtgcgtggacagactgacacacatcatcagaaagggaagaatctggatgcacaaggaaagacaggagaacgtgcagaagatcattctggaaggtacagagagaatggaagatcagggacagagcatcattcctatgctgactggagaggtgatccccgtgatggaactgctgagctccatgaaaagccactctgttcctgaggaaatcgacatcgccgacaccgtgctgaacgacgacgatattggagatagctgccacgagggcttccttctgaacgccatcagcagccacctgcagacatgcggctgcagcgtcgtggtgggctccagcgccgagaaggtgaacaagatcgtgcggaccctgtgcctgttcctgacccctgctgagcggaagtgcagtagactgtgtgaagccgagagcagcttcaagtacgagtccggcctgtttgtgcagggcctgctgaaggacagcacaggcagcttcgtgctgcccttcagacaagtgatgtacgccccttaccccaccacccacatcgacgtcgacgtgaacaccgtgaagcagatgcctccatgtcacgagcacatctacaaccagaggcggtacatgagatctgagctgaccgccttttggcgggccacaagcgaggaagatatggcccaggacaccatcatctacaccgacgagtctttcacccctgatctgaatatctttcaggatgtcctgcaccgggacacactggtgaaggccttcctggaccaggtgttccagctgaagcccggcctgtccctgcggagcaccttcctggcccaatttctgctcgtgcttcacagaaaggccctgacactgatcaagtacatcgaggacgacacccagaaaggcaagaagcctttcaagtccctgcgcaacctgaaaatcgatctggacctgaccgccgagggcgacctgaacatcatcatggcccttgccgagaaaatcaaacctggcctgcacagcttcatcttcggcagacctttttataccagcgtgcaggagagagatgtgcttatgaccttctga

SEQ ID NO: 24 - DNA, 1446 bp, synthetic construct
  misc_feature 1..1446: note = Description of Artificial Sequence: Synthetic polynucleotide
  source 1..1446: mol_type = other DNA
atgagcaccctgtgtcctccaccatctcctgccgtggccaagacagagatcgccctgtctggcaagtcacctctgctggccgctacattcgcctactgggacaacatccttggacctagagtgcggcacatctgggcccctaagaccgagcaggttctgctgagcgacggcgagataacatttctggccaaccacacacttaatggcgagatcctgagaaacgccgagtctggcgccatcgatgtgaagttcttcgtgctgtccgagaagggcgtgatcatcgtgtccctgatcttcgacggcaactggaacggcgaccggtctacctacggcctgtccatcatcctgccccagacagagctgagtttctacctgccactgcatagagtgtgcgtggacagactgacacacatcatcagaaagggcagaatctggatgcacaaggaacggcaggagaacgtgcagaagatcatcctcgagggcaccgagcggatggaagatcagggccagagcatcattcctatgctgacaggcgaagtgatccccgtgatggaactgctgtctagcatgaaaagccacagcgtgccggaagagatcgacatcgccgacacagtgctgaacgacgacgacatcggcgatagctgccacgagggcttcctcctgaacgccatcagctcccacctgcagacctgcggctgctctgtggtcgtgggctctagcgccgaaaaggtgaacaagatcgtgcggaccctgtgcctgttcctgacacctgctgaaagaaaatgcagcagactgtgtgaagccgagagcagcttcaagtacgagagcggcctgttcgtgcagggactcctgaaggacagcacaggcagctttgtgctgcctttcagacaggtgatgtacgccccctaccccaccacccacatcgacgtcgacgtgaacaccgtgaaacagatgcctccttgtcacgagcacatctacaaccagcggagatacatgagaagcgagctgacggccttttggcgggccacttccgaggaagatatggctcaggacacaatcatctacactgatgagtccttcacccctgatctgaatatctttcaggacgtgctgcacagagataccctggtgaaggccttcctggatcaggtctttcagctgaagcccggcctgtctctgagaagcaccttcctggcccagttcctgcttgtgctgcaccggaaggccctgaccctgatcaagtacatcgaggacgatacccagaaaggaaaaaagccttttaagagcctgcggaacctgaaaatcgacctggacctgaccgccgagggagatctgaacatcatcatggccctggctgaaaagattaagcctggactgcacagcttcatcttcggcagacctttctacaccagcgtgcaagagcgggacgtgctgatgaccttttga

SEQ ID NO: 25 - DNA, 1446 bp, synthetic construct
  misc_feature 1..1446: note = Description of Artificial Sequence: Synthetic polynucleotide
  source 1..1446: mol_type = other DNA
atgagcacactgtgccctccaccgagccctgctgtggccaagacagagatcgccctctctggcaagagccccctgttggccgccacattcgcctactgggacaacatcctgggtcctagagtgcggcacatctgggcccctaagaccgagcaggtgctgctgagtgatggagaaataacattcctggccaaccacaccctgaacggcgaaatcctgagaaacgccgagagcggtgctatcgacgtgaagttcttcgtgctcagcgagaagggagtgatcatcgtgtccctgatcttcgacggcaactggaacggcgaccggagcacctacggcctgagcatcatcctgcctcagaccgagctgagcttttacctgcctctgcacagagtgtgcgtggacagactgacccacatcattagaaagggcagaatctggatgcacaaggaacggcaggagaacgtgcagaagatcatcctcgagggtacagagagaatggaagatcagggccagtctatcatccctatgctgaccggcgaggtgatcccagtgatggaactgctgtccagcatgaagagtcactctgttcctgaggaaatcgacatcgccgacaccgtgctgaacgacgatgacatcggcgatagctgccacgagggcttcctgctgaatgccatcagcagccacctgcagacatgcggctgtagcgtggtggtcggcagcagcgccgaaaaagtgaacaagatcgtgcggaccctctgtctgttcctgacacctgccgagcgcaagtgcagcagactgtgtgaagccgaatccagcttcaagtacgagtctggactcttcgtgcaaggcctgctgaaggacagcaccggctcttttgtgctgcccttcagacaggtcatgtacgccccataccccaccacacacattgatgttgacgtcaacaccgtgaagcagatgcctccgtgccatgagcacatctacaaccagcggagatacatgagatctgagctgaccgccttttggcgggccaccagcgaagaggatatggctcaagacacaatcatctatactgatgagagcttcacccctgatctgaatatctttcaggacgtgctgcaccgagacaccctcgtgaaagccttcctggaccaggtgttccagctgaaacctggcctgtctctgagaagcaccttcctcgcccagttcctgctggtgctgcacagaaaggccctgacactgatcaagtacatcgaggacgacacccagaaaggcaagaaaccctttaagtccctgcggaatctgaagattgacctggatctgaccgccgagggcgacctgaacatcatcatggccctggccgagaagatcaagcccggcctccacagcttcatctttggcagacctttctacaccagcgtgcaggagagagatgtgctgatgaccttctga

SEQ ID NO: 26 - DNA, 1446 bp, synthetic construct
  misc_feature 1..1446: note = Description of Artificial Sequence: Synthetic polynucleotide
  source 1..1446: mol_type = other DNA
atgagcaccctgtgtcctccaccgagccctgctgtggccaagaccgagatcgccctgagcggcaaatctcctctgctggccgctacattcgcctactgggacaacatcctgggccctagagtgcggcacatttgggcccctaagaccgagcaggtgctgctgagcgacggcgaaatcacctttctggccaaccacaccctgaacggcgagatcctgcggaacgccgaaagcggcgccatcgacgtcaagttcttcgtgctgtctgagaagggcgtgatcattgtgtccctgatcttcgacggcaactggaacggcgacagaagcacctacggcctgtccatcatactgccccagaccgagctgtctttctacctgcctctgcaccgcgtgtgcgtggatagactgacccacatcattagaaaaggcagaatctggatgcacaaggaacggcaggagaacgtgcagaagatcatcctggaagggaccgaaagaatggaagatcagggacagagcatcatccccatgctgactggcgaggtgatccctgtgatggaactgctgagctctatgaaaagccacagcgtgcccgaggaaatcgatatcgctgataccgtgctgaacgacgatgacatcggcgatagctgccacgagggcttcctgctgaacgccatcagcagccacctgcagacatgcggctgtagcgtcgtggtgggctcttccgccgagaaggtgaacaagatcgtgcggaccctgtgcctgttcctgacacctgccgagagaaagtgcagcagactgtgcgaggccgaatcttcttttaagtacgagagcggactcttcgtgcaaggactgctgaaagacagcacaggcagctttgtgctgcctttcagacaggttatgtacgccccctaccccaccacccacatcgacgtggacgtgaacaccgtgaagcagatgcctccatgtcacgagcacatctacaaccagcggagatacatgagatctgaactgaccgcattctggcgggccaccagcgaagaggatatggcccaggacacaatcatctatacagacgagagcttcacccctgatcttaatatcttccaagacgtgctgcaccgggacaccctggtgaaagccttcctggatcaagtgttccagctgaagcccggcctgagcctgagatccacattccttgctcagttcctgctggtcctgcacagaaaggccctgacgctgatcaagtacatcgaggacgacacccagaaaggcaagaagcctttcaagagcctgagaaacctgaagatcgacctggacctgacagccgagggcgacctgaatatcatcatggccctggctgaaaagatcaagcctggactgcatagcttcatctttggaagacctttttacacctccgtccaagagcgggacgtgctgatgaccttctga

SEQ ID NO: 27 - DNA, 1446 bp, synthetic construct
  misc_feature 1..1446: note = Description of Artificial Sequence: Synthetic polynucleotide
  source 1..1446: mol_type = other DNA
atgagcacactgtgccctcctccaagccctgccgtggccaagaccgagatagctctgagcggcaagagccccctgcttgccgccacattcgcctactgggacaacatcctgggccccagagtgcggcacatctgggcccctaagacagagcaggtgctgctgagcgacggcgagatcaccttcctggccaaccacaccctgaatggcgaaatcctgagaaacgccgagagcggtgctatcgatgtgaagttcttcgtgttgtctgaaaagggcgtgatcatagtttctctgatctttgatggcaactggaacggcgatagatccacatacggcctctccatcatactcccccagacagagctgagcttctatctgcctctgcacagagtgtgcgtggacagactgacccacatcattagaaagggcagaatctggatgcacaaggaacggcaggagaacgtgcaaaagatcatcctggaaggtacagagcggatggaagatcagggccagtctatcattcctatgctgaccggcgaggtgatccccgtgatggaactgctgtctagcatgaaatcccacagcgtgccggaagaaatcgacatcgccgacaccgtgctgaacgacgatgacataggagatagctgccacgagggcttcctgctgaatgccatcagcagccacctgcagacctgcggctgcagcgtggtggtcggcagctccgccgaaaaggtgaacaagatcgtgcggaccctctgtctgttcctgacccctgctgaaagaaagtgcagtagactgtgtgaagccgagagctcttttaagtacgagtctggacttttcgtgcagggcctgctgaaggacagcacaggcagcttcgtgctgccttttagacaggtgatgtacgccccttaccccaccacccacatcgacgtggacgtcaacaccgtgaaacagatgcctccttgccatgagcacatctacaaccagagacggtacatgagaagcgagctgaccgccttctggcgggccaccagtgaagaggacatggcacaggataccatcatctatacagacgagtccttcacccctgacctgaacatcttccaggacgtgctgcacagagataccctggtcaaggcttttctggaccaggttttccagctgaagcctggcctgagcctgcggtccaccttcctggcccagttcctgctggtgctgcaccggaaggccctgaccctcatcaagtacatcgaggacgacacccagaaaggcaaaaagcctttcaagtccctgcgcaacctgaaaattgacctggatctgacagccgagggagatctgaatatcatcatggccctggccgagaagatcaagcccggcctgcatagcttcatcttcggccgccccttttacaccagcgtgcaggagagggacgtgctgatgacattctga

SEQ ID NO: 28 - DNA, 1446 bp, synthetic construct
  misc_feature 1..1446: note = Description of Artificial Sequence: Synthetic polynucleotide
  source 1..1446: mol_type = other DNA
atgagcacactgtgtcctccacctagccctgccgtggccaagaccgaaatcgccctgagcggaaagagccccctgctggccgccaccttcgcctactgggacaacatcctgggccctagagtgcggcacatctgggcccctaagaccgagcaggtcttgctttctgatggcgaaatcaccttcctcgctaatcacaccctgaacggcgagatcctgagaaatgccgagtccggcgccattgacgtgaagttcttcgtgctgagcgagaagggcgtgatcatcgtgtccctgatcttcgacggaaactggaacggcgacagaagcacctacggcctgtccatcatcctgcctcagaccgagctgagcttctacctgccactgcatagagtgtgcgtggaccggctgacacacatcatccggaagggcagaatctggatgcacaaggaacggcaggagaacgtgcagaaaatcatcctggaaggtacagagagaatggaagatcagggccagagcatcatccctatgctgaccggcgaggtgatccccgtgatggaactgctcagctctatgaagtcccacagcgtgcctgaggaaattgacatcgccgataccgtgctgaacgacgacgacatcggcgacagctgccacgagggcttcctgctgaacgccatcagcagccacctgcagacctgcggctgcagcgtggtggtcggcagctccgccgagaaggtgaacaagatcgtgcggaccctctgtctgttcctgactcctgctgaaagaaagtgcagtagactgtgcgaggccgaatctagcttcaagtacgagagcggcctttttgtgcagggactcctgaaggactctacaggctctttcgtgctgccttttagacaggtgatgtacgccccctaccccaccacccacattgacgtggatgtcaacacagtgaaacagatgcccccctgccacgagcacatctacaaccagaggcggtacatgcggagcgagctgaccgccttctggcgggccacaagcgaagaggacatggctcaagacaccatcatatatacagacgagagcttcacccctgatctgaatatctttcaggacgtgctgcaccgggacaccctggtcaaggcctttctggaccaggtgttccagctgaaacctggcctgagcctgaggtccaccttcttggcacagttcctgctggtgctgcacagaaaagccctgacactgatcaaatacatcgaggatgacacacagaagggaaaaaagcccttcaagtctctgagaaacctgaagatcgatctggatctgacagccgagggagatctgaacatcatcatggccctggctgaaaagatcaagcctggacttcattctttcatcttcggcagacctttctacaccagcgtgcaggagcgggacgttctgatgaccttttga

SEQ ID NO: 29 - DNA, 1446 bp, synthetic construct
  misc_feature 1..1446: note = Description of Artificial Sequence: Synthetic polynucleotide
  source 1..1446: mol_type = other DNA
atgagcaccctgtgccccccccccagccctgccgtggccaagaccgagatcgccctctccggcaagtcccctctgctggccgctacatttgcctactgggacaacatcctcggccctagagtgcggcacatttgggcccctaagaccgaacaggtcctcctgagcgacggcgaaataacatttctggccaaccacaccctgaacggcgaaatcctgagaaacgccgagagcggcgccatcgacgtgaagttcttcgtgctgtccgagaaaggcgtgatcatcgtgtccctgatcttcgacggcaactggaacggagatagaagcacatacggactgagcatcatcctcccacagaccgagctgtctttctacctgcctctgcaccgggtgtgcgtggacagactgacccacatcattagaaagggcagaatctggatgcacaaggaacggcaggagaacgtgcaaaagatcatcctggaagggaccgagcgtatggaagatcagggccagagcatcattcctatgctgaccggcgaggtgatccccgtgatggaactgctgagcagcatgaaaagccactctgtgcccgaggaaatcgacatcgccgacactgtgttgaacgacgatgatatcggcgatagctgccacgagggcttcctgctgaacgccatcagctcccacctgcagacatgcggctgtagcgttgtggtgggctctagcgccgaaaaagtgaacaagatcgtgcggaccctttgcctgttcctgacacctgctgagagaaagtgcagcagactgtgtgaagccgaatctagctttaagtacgagtccggactcttcgtgcaaggcctgctcaaggacagcacaggcagcttcgtgctgcctttcagacaggtgatgtacgccccttaccccaccacccacatcgatgtcgacgtgaacaccgtgaagcagatgcctccttgccacgagcacatctacaaccagagacggtacatgagaagcgagctgaccgccttttggcgggccaccagcgaagaggacatggctcaagatacaatcatctataccgacgagagctttacccctgatctgaacatctttcaggacgtgctgcacagagataccctggtgaaagccttcctggatcaggtgttccagctgaagcctggcctgtctctgcgatctacattcctcgctcagttcctgctggtcctgcatagaaaggccctgactctgatcaagtacatcgaggacgacacacagaagggcaaaaagcccttcaagtctctgcggaacctgaaaatcgacctggacctgaccgccgagggcgacctgaatatcatcatggccctggccgagaagatcaaacccggcctgcacagcttcatcttcggaagacctttctacaccagcgtgcaggagagagacgtgctgatgaccttctga

SEQ ID NO: 30 - DNA, 1446 bp, synthetic construct
  misc_feature 1..1446: note = Description of Artificial Sequence: Synthetic polynucleotide
  source 1..1446: mol_type = other DNA
atgagcaccctgtgtcctccaccgagccctgccgtggccaagaccgagatagctctgtccggcaagtccccactgctggccgccaccttcgcctactgggacaacatcctgggacctagagtgcggcacatctgggcccctaagacggagcaggtcctgctgagcgacggcgaaataacattcctggctaatcacaccctgaatggcgagatcctgagaaacgccgaaagcggcgccatcgacgtgaagttcttcgtgctgtctgaaaagggagtgatcatcgtgtccctgatcttcgacggcaactggaacggcgaccggtctacctacggcctgagcatcatcctgccccagaccgaactgtctttttacctgcctctgcacagagtgtgcgtggacagactgacccacatcatccggaagggaagaatctggatgcacaaggaacggcaggagaacgtgcaaaagatcattctcgagggcaccgagagaatggaagatcagggccagagcatcatccccatgctgaccggcgaggtgatccctgtgatggaactgctgagcagcatgaagtcccactctgtgcctgaggaaatcgacatcgccgatacagtgctgaacgacgacgatatcggcgacagctgccacgagggcttcctgctgaacgccatcagctctcacctgcagacatgcggctgcagcgtggtggtgggcagcagcgccgagaaggtgaacaagatcgtgcggaccctttgcctgttcttgacccctgctgagagaaagtgcagcagactgtgtgaagccgaatctagctttaagtacgagtctggcctcttcgtgcagggactgctgaaggacagcacaggcagcttcgtgctgccttttagacaggtgatgtacgccccttaccctacaacacacattgacgtggacgttaacaccgtgaaacagatgcctccatgtcacgagcacatctacaaccagagacggtacatgcggagcgagctgacagccttttggcgggccacaagcgaggaagatatggcccaagacacaatcatctatacagacgagagcttcacccctgacctgaacatctttcaggacgtgctccatagagataccctggtgaaggccttcctggaccaggtgttccagctgaagcccggactgagcctgagatctacattcctggcccagttcctgctggtgctgcacagaaaggccctgacactgatcaagtacatcgaggatgatacacagaaaggcaaaaagcctttcaagagcctgcggaacctgaaaatcgacctggatctgaccgccgagggagatctgaacatcatcatggccctggccgaaaagatcaagcccggcctgcacagcttcatcttcggcagacccttctacaccagcgtgcaggagcgggacgttctgatgaccttttga

SEQ ID NO: 31 - DNA, 1446 bp, synthetic construct
  misc_feature 1..1446: note = Description of Artificial Sequence: Synthetic polynucleotide
  source 1..1446: mol_type = other DNA
atgagcaccctgtgccccccccccagccccgccgtggccaagaccgagatcgccctgtctggaaagagccctctgctggccgctacattcgcctactgggacaacatcctgggccctagagtgcggcacatctgggcccctaagaccgaacaggtgctgctgagtgatggcgagatcaccttcctggccaaccacaccctgaatggagaaatcctgagaaatgccgaaagcggcgccatcgacgtgaagttcttcgtgctgagcgagaagggcgtgatcatcgtgtccctgatcttcgacggcaactggaacggcgatagaagcacatacggcctgtctatcatcctgcctcagacagagctgagcttctacctgcccctgcaccgggtgtgcgtggacagactgacacacattatccggaaaggcagaatctggatgcacaaggaacggcaggagaacgtgcagaaaatcatcctggaaggtacagaacggatggaagatcagggccagagcatcattcctatgctgaccggcgaggtgatccccgtgatggaactgctatccagcatgaaaagccactctgtgcctgaggaaatcgatatcgccgacaccgtgctgaacgacgacgacatcggcgactcttgtcacgagggcttcctgctcaatgctatcagcagccacctgcagacctgcggctgttctgtggtcgtgggcagctccgccgaaaaggtgaacaagatagttagaaccctgtgcctgttcctgacccctgccgagcggaagtgcagcagactgtgtgaagccgagtccagctttaagtatgagagcggactgttcgttcaaggcctgctcaaggacagcaccggctcttttgtgctcccttttagacaggtcatgtacgccccttaccccacaacacacatcgacgttgacgtgaacaccgtgaagcagatgcctccttgccacgagcacatctacaaccagagacggtacatgcggagcgagctgaccgccttttggcgggccacatctgaagaggacatggcccaggacaccatcatctacaccgacgagagcttcacacctgacctgaatatcttccaagacgtgctgcacagagacaccctggtgaaagccttcctggatcaggtgttccagctgaaacctggcctgtccctgcggagcacctttctggcccaatttctgctcgtgcttcatagaaaggccctgacgctcatcaagtacatcgaggatgacacacagaagggcaaaaagcctttcaagtccctgagaaacctgaagattgatctggacctgaccgccgagggagatctgaacatcatcatggccctggctgagaagattaagcccggcctgcacagcttcatcttcggcagacctttctacacaagcgtgcaggagcgggacgtcctcatgaccttctga

SEQ ID NO: 32 - DNA, 1446 bp, synthetic construct
  misc_feature 1..1446: note = Description of Artificial Sequence: Synthetic polynucleotide
  source 1..1446: mol_type = other DNA
atgagcacactctgccctcctcctagccctgccgtggccaagaccgagatcgccctgagcggaaagtctccactgctggccgctacattcgcctactgggacaacatactgggccctagagtgcggcacatctgggcccctaagaccgagcaggtcctcctgagtgatggagaaatcacctttctggctaatcacaccctgaacggcgagatcctgaggaacgccgaaagcggcgccatcgacgtgaagttcttcgttctgagcgagaagggagtgatcatcgtgtccctgatcttcgacggcaactggaacggcgatagatctacatacggcctgagcatcatcctgcctcagacagagctgtctttctacctgcctctgcacagagtttgtgtggaccggctgacccacatcatcagaaaaggccggatctggatgcacaaggaacggcaggagaacgtgcagaaaatcatcctggaaggcaccgagcggatggaagatcagggccagagcatcattcctatgctgacaggcgaggtgatccccgtgatggaactgctgtcttctatgaaaagccactctgtgcccgaggaaatcgacatcgccgacaccgtgctcaacgacgacgatatcggcgactcttgtcacgaaggcttcctgctgaatgccatcagcagccacctgcagacctgcggctgttctgtcgtggtgggctccagcgccgaaaaggtgaacaagatagttagaaccctgtgcctgttcctgacccctgctgaaagaaagtgcagcagactgtgcgaggccgagagcagcttcaagtacgagagcggcctgtttgtgcaaggcctgctgaaggacagcaccggcagcttcgtgctgcccttcagacaggtgatgtacgccccttatcctaccacccacatcgacgtggacgtgaacaccgtgaagcagatgcccccctgccacgagcacatctacaaccagagaagatacatgagaagcgagctgaccgccttctggcgggccaccagcgaggaagatatggcccaagatacaatcatctacaccgacgagagctttacacctgatctgaacatctttcaggacgtgctgcaccgggacaccctggtcaaggcctttctggatcaggtgttccagctgaagcctggactgagcctgaggtccaccttcctggcccagttcctgctggtgctgcatagaaaggccctgaccctgatcaagtacatcgaggacgacacacagaagggcaagaagccctttaagtccctgcggaacctgaaaatcgacctggacctgacagccgagggcgacctgaacatcatcatggctctggctgagaagatcaaacccggcctgcacagcttcatcttcggcagacctttttacacaagcgtgcaagagagagatgtgctgatgaccttctga

SEQ ID NO: 33 - DNA, 1446 bp, synthetic construct
  misc_feature 1..1446: note = Description of Artificial Sequence: Synthetic polynucleotide
  source 1..1446: mol_type = other DNA
atgagcacactgtgtcctcctccgagccctgccgtggccaagaccgagatcgccctgagcggcaagtccccactgcttgctgctaccttcgcctactgggacaacatcctgggccctagagtgcggcacatctgggcccctaagacagagcaggtgctgctgagcgacggcgaaataacattcctggccaaccacaccctgaacggcgagatcctgagaaacgccgagagcggcgctatcgacgtgaagttcttcgttctgtctgaaaagggcgtgatcatcgtgtccctgatcttcgacggcaactggaacggcgatagaagcacctacggcctgagcattatcctgcctcagacagaactgtctttctacctgcctctgcacagagtgtgcgtggacagactgacacacatcattagaaagggcagaatctggatgcacaaggaaagacaggagaacgtgcagaagatcatcctggaaggcaccgagagaatggaagatcagggccagtctatcatccctatgctgaccggcgaggtgatccccgtgatggaactgctgtctagcatgaaaagccactctgtgcccgaggaaatcgacatcgccgatacagtgctgaacgacgatgatataggagatagctgccatgagggcttcctgctgaacgccatcagctcccacctgcagacctgcggatgtagcgtggtcgtgggctcctccgccgagaaggtgaacaagatcgtgcggaccctgtgcctgttcctgacacctgctgaacggaagtgcagcagactgtgcgaggccgaatcttcttttaagtacgagagcggactgttcgtgcaaggcctgctgaaggacagcaccggcagctttgtgctgccattccggcaggtgatgtacgccccttaccccaccacccacattgacgtcgacgtgaacaccgtgaagcagatgcccccctgtcacgagcacatctacaaccagaggcggtacatgagaagcgagctgacagccttttggcgggccaccagcgaggaagatatggcccaagacaccatcatctacaccgacgagagcttcacccctgatctgaatatctttcaggacgtgctgcacagagatacactggtgaaagccttcctggaccaggttttccagctgaagcctggcctgagcctgcgcagcacctttctggcccagttcctgctcgtgctgcaccggaaggccctgacactgattaagtacatcgaggacgacacccagaaaggaaaaaagcccttcaagagcctgcggaacctgaaaatcgacctggacctgaccgccgagggcgacctgaacatcatcatggccctggccgaaaagatcaaacctggactgcattctttcatcttcggcagacctttttacaccagcgtgcaggagcgggacgttctgatgaccttctga

SEQ ID NO: 34 - DNA, 1446 bp, synthetic construct
  misc_feature 1..1446: note = Description of Artificial Sequence: Synthetic polynucleotide
  source 1..1446: mol_type = other DNA
atgtctacactctgtcctccacctagccctgctgtggccaagacagaaatcgccctgagcggaaaaagccccctgctggccgccaccttcgcctactgggacaacatcctgggccccagagtcagacacatctgggcccctaagaccgagcaggtgctgctgagcgacggagagatcaccttcctggccaaccacaccctgaatggcgagatcctgcggaacgccgagtctggcgccatcgacgtgaagttcttcgtgctgtctgagaaaggcgtgatcattgtgtccctcatctttgacggcaactggaacggagatagaagcacctacggcctgtccatcatcctgccccagacagagctgagcttctacctgcctctgcacagagtgtgcgtggacagactgacccacatcatcagaaagggcagaatctggatgcacaaggaacggcaggagaacgtgcaaaaaatcatcctggaaggcaccgagagaatggaagatcagggccagagcatcatccccatgctgaccggcgaggtgatccctgtgatggaactgctgagcagcatgaagtcccattctgtccccgaggaaatcgacatcgccgacaccgtgctgaacgacgatgatatcggcgatagctgccacgagggcttcctgctgaacgccatcagctctcacctgcagacctgcggctgcagcgtggtggtcggctcttccgccgaaaaggtgaacaagatcgtgcggaccctgtgcctgttcctgactcctgccgaaagaaagtgctctagactgtgtgaagccgagagcagcttcaaatacgagtccggtctttttgtgcaggggctgctgaaggacagcacaggcagcttcgtgcttccattcagacaggtgatgtacgccccttaccccacaacacacattgatgtggacgtgaacaccgtgaagcagatgcctccttgccacgagcacatctacaaccagcggagatacatgcggagcgagctgacagccttctggcgggccacaagcgaggaagatatggcccaggacaccatcatctacaccgacgagagcttcacccctgatctgaatatcttccaagacgtcctgcaccgcgacacactcgtgaaagcctttctcgaccaggttttccagctgaaacctggcctgagtctgagatccaccttcctggctcaatttctgctggtgctccaccggaaggccctgaccctgatcaagtacatcgaggacgacacccagaagggcaagaagcctttcaagtctctgagaaacctgaagatcgacctggacctgacagctgagggcgacctgaatatcatcatggcccttgctgagaagatcaagcccggcctgcacagcttcatcttcggcagacctttttataccagcgtgcaggagagagatgtgctgatgaccttctga

SEQ ID NO: 35 - DNA, 1446 bp, synthetic construct
  misc_feature 1..1446: note = Description of Artificial Sequence: Synthetic polynucleotide
  source 1..1446: mol_type = other DNA
atgagcaccctgtgtcctccacctagccccgccgtggccaagaccgagatcgccctgtctggaaagtcccctctgctggccgctacattcgcctactgggacaacatcctgggacctagagtgcggcacatctgggcccctaagaccgagcaggtgctcctgagtgatggcgagataacatttctggccaaccacaccctcaacggcgagatcctgagaaacgccgaaagcggcgccatcgacgtgaagttcttcgtgctgtctgaaaagggcgtgatcatcgtgtccctgatcttcgacggcaactggaacggcgacagaagcacgtacggcctgtccatcatcctgccccagaccgagctgtctttctacctgcctctgcaccgggtgtgcgtggatagactgacccacattattagaaagggcagaatctggatgcacaaggaacgccaggagaacgtgcagaagatcatcctggaaggtacagagcggatggaagatcagggccagagcatcatccccatgctgaccggcgaagtgatccctgtgatggaactgctgagttctatgaaaagccacagcgtgccggaagagatcgatatcgccgacaccgtccttaacgacgacgacataggagatagctgccacgagggcttccttctgaacgccatcagctctcacctgcagacatgcggctgcagcgtcgtggtcggctctagcgccgaaaaagtgaacaagatcgtgcggaccctgtgcctgttcctgacacctgccgagagaaagtgctctagactgtgcgaggccgagtccagcttcaagtacgagagcggcctgtttgttcaaggactgctgaaggacagcaccggcagctttgtgctcccttttagacaggtgatgtacgccccttaccccaccacccacatcgacgttgacgtgaataccgtgaaacagatgcctccttgtcacgagcacatctacaaccagagaagatacatgagatctgagctgaccgccttctggcgggccaccagcgaggaagatatggcccaggacaccatcatctacaccgacgagagcttcacccctgatctgaacatctttcaggatgtcctgcaccgcgacaccctggtcaaagcctttctggaccaggtgttccagctgaaacccggactgtctctgcggagcaccttcttggctcaatttctcctggtgctgcacagaaaggccctgacactgatcaagtacatcgaggatgatacacagaaaggcaaaaagcccttcaagagcctgagaaatctgaagatcgacctggacctgacagccgagggcgatctgaacatcatcatggccctggctgagaagattaagcctggcctccattctttcatcttcggcagacctttctacaccagcgtgcaggagcgggacgtgctgatgacattctga

SEQ ID NO: 36 - DNA, 1446 bp, synthetic construct
  misc_feature 1..1446: note = Description of Artificial Sequence: Synthetic polynucleotide
  source 1..1446: mol_type = other DNA
atgagcaccctgtgtcctcctccatctccagccgtggccaagaccgagatcgccctgtccggcaagagccctctgctggccgctacattcgcctactgggacaacatcctgggacctagagtgcggcacatctgggcccctaagacagagcaggtgctgctgagtgatggcgagatcaccttcctggccaaccacaccctgaatggagaaatcctgagaaacgccgagagtggcgccatcgatgtgaagttcttcgtgctgtctgaaaagggcgtgatcatcgtcagcctgatcttcgacggcaactggaacggcgacagaagcacatacggcctgagcatcatcctgccccagacagagctgtctttttacctgcctctgcacagagtgtgcgtggaccggctgacccacatcattagaaagggcagaatctggatgcacaaggaaagacaggagaacgtgcagaagatcatcctggaaggtacagagagaatggaagatcagggacagagcatcatccccatgctgaccggcgaagtgatccctgtgatggaactgctgagcagcatgaaaagccattctgtgcccgaggaaatcgacatcgccgacacagtgctgaacgacgacgatatcggcgatagctgccacgagggattcctgcttaatgccatcagcagccacctgcagacctgtggctgtagcgtggtcgtgggcagctccgccgagaaggtgaacaagatcgtgaggaccctctgcctgttcctgacacctgctgaaagaaagtgcagcagactgtgcgaggccgagtccagcttcaagtacgagagcggcctcttcgtgcagggcctgctgaaggacagcaccggctccttcgtgctgccttttagacaggtgatgtacgccccttaccccaccacccacattgacgtggacgtgaacaccgtgaagcagatgcctccgtgccacgagcacatctacaaccagcgcagatacatgcggagcgagctgaccgccttctggcgggccacatctgaggaagatatggctcaagataccatcatctacaccgacgagagcttcacccctgatctgaacatcttccaggacgtgctgcatagagataccctggtgaaagctttccttgatcaggttttccaactgaagcctggcctgagcctgagaagcaccttcctggctcagttcctgctggtgcttcaccggaaggccctaaccctgatcaagtacatcgaggatgacacccagaaaggcaaaaagccttttaagtccctgcggaacctgaaaatcgacctggacctcacagccgagggagatctgaacatcatcatggccctggccgaaaagataaagcccggcctgcacagcttcatctttggcagacctttctacacaagcgtgcaggagcgggacgtgctgatgaccttctga

SEQ ID NO: 37 - DNA, 1446 bp, synthetic construct
  misc_feature 1..1446: note = Description of Artificial Sequence: Synthetic polynucleotide
  source 1..1446: mol_type = other DNA
atgagcaccctctgtcctccacctagccctgctgtggccaagaccgaaattgccctgagcggaaagtctcctctgttggctgctacattcgcctactgggacaacatcctgggccctagagtgcggcacatctgggcccctaagacagagcaggtgctgctgagtgatggcgaaatcaccttcctggccaaccacaccctgaacggcgagatcctgagaaacgccgaaagcggcgccatcgacgtgaagttcttcgtgctgtctgaaaagggtgttatcattgtgtccctgatctttgacggcaactggaacggcgacagatctacatacggcctgtccatcatcctgcctcagaccgagctgtctttctacctgcctctgcacagagtgtgcgtggaccggctgactcatatcatcagaaagggaagaatctggatgcacaaggaaagacaggagaacgtgcagaagatcatcctggaaggtacagagagaatggaagatcagggccagagcatcatccccatgctgacaggcgaggtgatccctgtgatggaactgctgagcagcatgaagtcccacagcgtccccgaggaaatcgacatcgccgacacagtgctgaacgacgacgatatcggcgattcatgccacgagggcttcctgctgaatgcaatcagcagccacctgcagacctgcggctgttctgtggtggtgggcagcagcgccgaaaaagtgaacaagatcgtgcgcaccctgtgcctgtttttgacccctgccgagcggaagtgcagcagactgtgtgaagccgagagctctttcaagtacgagagcggcctgttcgttcaaggcctgctgaaggacagcaccggcagctttgtgctgcccttccggcaggtgatgtacgccccttaccccaccacccacatcgacgtcgacgtgaacaccgtgaagcagatgcctccgtgccacgagcacatctacaaccagcggagatacatgcggtccgagctgacagccttctggcgggccaccagcgaagaggacatggcccaggacaccatcatctacactgatgagtccttcacacctgatctgaatatcttccaagacgtgcttcacagagacaccctggtgaaagcttttctcgaccaggttttccagctgaagcccggcctgagcctgagatctaccttcctggctcaatttctgctcgtgctgcacagaaaggccctgacgctgatcaagtatatcgaggacgacacgcagaaaggcaagaaacccttcaaaagcctgcggaacctgaaaattgacctggacctgaccgccgagggcgacctgaacatcatcatggccctggccgagaagatcaagcctggactgcatagcttcatcttcggcagacctttttacacctctgtgcaggagcgggacgtgctcatgaccttttga

SEQ ID NO: 38 - DNA, 1446 bp, synthetic construct
  misc_feature 1..1446: note = Description of Artificial Sequence: Synthetic polynucleotide
  source 1..1446: mol_type = other DNA
atgagcaccctgtgtcctcctccaagccctgccgtggccaagacagagatcgcccttagcggaaagtcccctctgctggccgccacatttgcctactgggacaacatcctgggacctagagtgcggcacatttgggccccaaagaccgagcaggtgctgctgagcgacggcgaaatcaccttcctggctaatcacacactgaacggcgagatcctgaggaacgccgaaagcggcgccatcgacgtgaagttcttcgtcctgagcgagaagggcgtgatcattgtgtccctgatcttcgacggcaactggaacggcgaccgctccacatacggcctgtctatcatcctgccccagaccgagctgtctttttacctgcctctgcacagagtgtgcgtggacagactgacccacatcatccggaagggcagaatctggatgcacaaggaacggcaggagaacgtgcagaaaatcatcctggaaggaacagagcggatggaagatcagggccagagcatcatacccatgctgactggcgaggtgatccctgtgatggaactgctgtcaagcatgaaaagccactctgtccccgaggaaatcgacatcgctgataccgtgctcaacgacgacgatatcggcgatagctgccacgagggcttcctgctgaacgccatcagcagccacctgcagacatgcggctgcagcgtcgtggtgggctctagcgccgaaaaggtgaacaagatcgtgcggaccctgtgtctgttcttgacccctgctgaaagaaagtgcagcagactgtgcgaggccgagagcagcttcaagtacgagtctggcctgtttgtgcagggcctgctgaaagacagcacaggcagcttcgtgctgcccttcagacaggtgatgtacgccccttaccctaccacccacattgacgtggacgtgaacaccgtgaagcagatgcctccgtgccacgagcacatctacaaccagcgtagatacatgagatccgagctgacagctttctggcgggccacctctgaagaggatatggcccaggacaccatcatctataccgacgagagcttcacccctgatctgaatatcttccaagacgtgctgcatagagacaccctggtgaaagccttcctggatcaagtgttccagctgaagcctggactgagcctgcggagcaccttcctggcccagttcctgctcgtgcttcatagaaaggccctgacactgatcaagtacatcgaggacgacacacagaagggcaaaaagcccttcaagagcctgagaaacctgaagatcgacctggacctgaccgccgagggcgatctgaacatcatcatggctctggccgagaagatcaagcccggcctgcacagctttatctttggcagacctttctacaccagcgtgcaagagagagatgtgctgatgaccttttga

SEQ ID NO: 39 - DNA, 1446 bp, synthetic construct
  misc_feature 1..1446: note = Description of Artificial Sequence: Synthetic polynucleotide
  source 1..1446: mol_type = other DNA
atgtctaccctgtgtcctcctccaagccccgccgtggccaagactgagatcgccctgagcggcaaatctcctctgctcgctgctaccttcgcctactgggacaacatcctgggacctagagtgcggcacatctgggcccctaagaccgagcaggtcctgctgagcgacggagagataacatttctggccaaccacacactgaacggcgagatcctcagaaatgccgagagcggcgccatcgacgtgaagttcttcgtgctgtctgagaagggcgtgatcattgtgtccctgatcttcgacggcaactggaacggcgacagaagcacctacggcctgagcatcatcctgcctcagacagagctgtccttttacctgccactgcaccgggtgtgcgtggatagactgacacacatcattagaaagggcagaatctggatgcacaaggaaagacaggagaacgtgcagaaaatcatcctggaaggtacagagcggatggaagatcagggccagagcatcatccctatgctgaccggcgaggtgatccccgttatggaactcctgtcttctatgaaaagccacagcgtccccgaggaaatcgacatcgcagatacagtgctgaacgacgacgatataggagatagctgtcacgagggcttcctgttaaacgccatcagcagccacctgcagacctgtggctgcagcgtggtggtcggctctagcgccgaaaaggtgaacaagatcgtgcggaccctgtgcctgttcctgacacctgctgaacggaagtgcagcagactgtgcgaggccgagagcagttttaagtacgagtccggcctgttcgtgcaaggcctgctgaaggactctacaggcagcttcgtgctgcctttcagacaggtgatgtacgccccttaccccaccacccacatcgacgtggacgtgaacaccgtgaagcagatgcctccgtgccacgagcacatctacaaccagcggagatacatgcggagcgagctgaccgctttctggcgggccaccagcgaagaggacatggctcaggacaccatcatctatacagacgagagcttcacccctgacctgaatatctttcaagacgtgctgcacagagataccctcgtgaaagccttcctggaccaggtgttccagctgaaacctggactgtcactgagaagcacctttctggcccagttcctgctggtcctgcacagaaaggccctgacccttatcaagtacatcgaggatgacacccagaagggcaagaagcccttcaagagcctgagaaacctgaagatcgacctggatctgacagccgaaggcgacctgaacatcatcatggccctggccgaaaagattaagcctggcctgcattctttcatcttcggccgccccttctacaccagcgtgcaggagagagatgtgctgatgaccttctga

SEQ ID NO: 40 - DNA, 1446 bp, synthetic construct
  misc_feature 1..1446: note = Description of Artificial Sequence: Synthetic polynucleotide
  source 1..1446: mol_type = other DNA
atgagcaccctgtgtcctcctcctagccctgccgtggcaaagaccgagatcgccctgagcgggaagtcacccctgctggccgctacatttgcctactgggacaacatcctgggccctagagtgcggcacatctgggcccctaagaccgagcaggtgctgctcagtgatggcgagataacattcctcgccaaccacacactgaatggcgaaatccttagaaatgccgagagcggtgctatcgacgtaaagttcttcgtgctgtctgaaaagggcgtgatcatcgtgtccctgatcttcgacggcaactggaacggcgatagaagcacctacggcctgagcatcatcctgcctcagacagagctgagcttctatctgcctctgcacagggtgtgcgtggacagactgactcacattattagaaaaggcagaatctggatgcacaaggaaagacaggagaacgtgcaaaagatcatcctggaaggcaccgagagaatggaagatcagggccagagcatcatccctatgctgaccggcgaggtgatccccgtgatggaactgctgagttctatgaagagtcactctgtgcccgaggaaatcgacatcgccgacacagtgctgaacgacgacgatatcggcgactcctgccacgagggcttcctgctgaacgccatcagcagccacctgcagacctgcggctgcagcgtggtggtcggcagctccgccgaaaaggtgaacaagatcgtgcggaccctgtgcctgttcctgacgcccgccgaaagaaagtgcagtagactgtgcgaggccgaaagctctttcaagtacgagagcggcctgtttgtgcagggcctgctcaaggacagcactggatctttcgtgctccccttcagacaggtgatgtacgccccttaccctacaacacacatcgatgtggacgtgaacaccgtgaagcagatgcctccatgtcacgagcacatctacaaccagcgtagatacatgagaagcgagctgacagccttttggcgggccacaagcgaggaagatatggcccaggacaccatcatctacaccgacgagagcttcacccctgacctgaatatctttcaggacgttctgcaccgggacacccttgtgaaggccttcctggaccaggttttccagctgaaacctggcctctccctgcggagcacattcctggctcagttcctgctggtgctgcatagaaaggccctgacactgatcaagtacatcgaggatgacacccagaagggcaaaaagccttttaagagcctgagaaacctgaagatcgacctggatctgaccgccgagggcgacctgaacatcatcatggctctggccgagaaaatcaagcccggactgcatagcttcatcttcggaagacctttctacaccagcgtgcaggagcgggacgtgctgatgaccttctga

SEQ ID NO: 41 - DNA, 1446 bp, synthetic construct
  misc_feature 1..1446: note = Description of Artificial Sequence: Synthetic polynucleotide
  source 1..1446: mol_type = other DNA
atgagcacactgtgcccccccccgagcccggccgtggccaagacagagatcgccctgagcggcaagtcccctctgctggccgccaccttcgcctactgggacaacatcctgggccctagagtgcggcacatctgggcccctaagaccgagcaggttctgctgagtgatggcgagataacattcctggccaaccacaccctgaacggcgagatcctgagaaatgccgaatctggcgccatcgacgtgaagttcttcgtgctgtctgagaagggcgtgatcattgtgtccctgatcttcgacggcaactggaacggcgatagaagcacctacggcctgagcatcatcctgccacagaccgaactgtcgttctacctgcctctgcaccgagtgtgcgtggacagactgacccacatcatcagaaagggaagaatctggatgcacaaggaaagacaggagaacgtgcagaagatcatcctggaaggtacagaacggatggaagatcagggacagagcatcatccccatgctgacaggcgaagtgatccctgtgatggaactgctgagctctatgaaaagccacagcgtgcctgaggaaatcgacatcgctgataccgtgctgaacgacgacgatatcggcgacagctgccacgagggcttcctgctgaacgccatcagcagtcacctgcagacatgcggctgtagcgtcgtggtgggctccagcgccgagaaagtgaacaagatcgtgcgcaccctgtgcctgttcctgacccctgctgagcggaaatgcagcagactgtgtgaagccgagagctcctttaagtacgagagcggcctttttgtgcagggcctgctgaaggacagcacaggcagcttcgtgctgcccttccggcaggtgatgtacgccccttatcctaccacccacatcgacgtcgacgtgaacaccgtgaagcagatgcctccttgccacgagcacatctacaaccagagaagatacatgagatccgagctgaccgccttctggcgggccacaagcgaggaagatatggcccaagacaccatcatctacactgatgagagtttcacccctgatctgaacatctttcaggacgtgctccatcgggacaccctggtgaaagctttcctggatcaagtctttcagctgaagcccggcctgtccctgcggtccaccttcctggcccagttcctgctcgtgctgcaccggaaggccctgaccctgatcaaatacatcgaggacgacacacagaaaggcaaaaagcctttcaagagcctgagaaacctgaaaatcgatctggacctgacagccgagggcgacctgaatatcatcatggccctggctgaaaagattaagcccggactgcattctttcatcttcggcagacctttctacaccagcgtgcaggagagagatgtcctcatgaccttttga

SEQ ID NO: 42 - DNA, 1446 bp, synthetic construct
  misc_feature 1..1446: note = Description of Artificial Sequence: Synthetic polynucleotide
  source 1..1446: mol_type = other DNA
atgagcacattgtgtcctccaccatctcctgccgtggccaagaccgaaatcgccctgagcggcaagagccccctgctcgccgccaccttcgcctactgggacaacatcctgggccctagagtgcggcacatctgggcccctaagaccgagcaggttctgctgagcgacggcgagataacattcctggctaatcacaccctgaatggcgagatcctgcggaacgccgaaagcggagccatcgacgtgaagttcttcgtgctgagcgagaagggagtgatcatcgtgtccctgatcttcgacggcaactggaacggcgaccgctccacctacggcctgtctatcatcctgcctcagaccgagctgagtttctacctgcctctgcaccgggtgtgcgtggacagactgacacacatcatccggaaaggcagaatctggatgcacaaggaacggcaggagaacgtgcaaaagatcatcctggaaggcaccgagagaatggaagatcagggccagagcatcattcccatgctgactggagaagtgatccctgtgatggaactgctgagcagcatgaagtcccacagcgtgcccgaggaaatcgacatcgccgacaccgtgctgaacgacgatgacataggagattcatgccacgagggcttcctgctgaacgccatcagctctcacctgcagacatgcggctgtagcgtcgtggtgggctctagcgccgaaaaggtgaacaagatcgtcagaaccctgtgcctgttcctgacccctgctgaaagaaagtgcagccggctgtgcgaggccgagtccagttttaagtacgagagcggcttgtttgtgcagggactgctgaaggacagcaccggcagcttcgtgctccccttcagacaggtgatgtacgccccttatcctacaacccacattgatgtggatgttaacaccgtgaagcagatgcctccatgtcatgagcacatctacaaccagcgtagatacatgcggagcgagctgaccgccttttggcgggccacaagcgaggaagatatggcccaggataccatcatctacacagacgagagcttcacccctgatctgaatatcttccaagacgtcctgcacagagacaccctcgtgaaggccttcctggaccaggtgttccagctgaaacccggcctgagcctgagaagcaccttcctcgctcagttcctgctggtgctgcatagaaaggccctgaccctgatcaagtacatcgaggacgacacacagaaaggaaaaaagcccttcaagagcctgagaaacctgaagatcgacctggatctgacagccgagggcgatctgaacatcatcatggctctggccgagaagatcaagcctggcctccactccttcatcttcggcagacctttttacaccagcgtgcaagagcgggacgtgctcatgaccttttga

SEQ ID NO: 43 - DNA, 1446 bp, synthetic construct
  misc_feature 1..1446: note = Description of Artificial Sequence: Synthetic polynucleotide
  source 1..1446: mol_type = other DNA
atgagcaccctgtgccccccccccagcccagccgtggccaagaccgagatagctctgagcggaaaaagccctctgctggccgccaccttcgcctactgggacaacatcctggggcctagagtcagacacatctgggcccctaagaccgagcaggtgctgctgagcgacggagagatcaccttcctggctaatcacaccctgaatggcgagatcctgagaaacgccgaaagcggcgccatcgacgtgaagttcttcgtgctgtctgaaaagggcgtgatcatcgtcagcctgatcttcgacggcaactggaacggcgacagaagcacatacggcctgtctatcattctgcctcagacagagctgagtttttacctgcctctgcaccgggtgtgcgtggaccggctgacccacatcattagaaagggaagaatctggatgcacaaggaacggcaggagaacgtgcagaaaatcatcctggaagggaccgagagaatggaagatcagggccagagcatcatccccatgctgaccggcgaagtgatccctgtgatggaactgctgtcttctatgaaaagccactctgtgcccgaggaaatcgatatcgccgatacagtgctgaacgacgacgacatcggcgactcatgccacgagggcttccttctgaacgccatcagctctcacctgcagacctgtggctgcagcgtggtcgtgggcagcagcgccgagaaagtgaacaagatcgtgcggaccctgtgtctgttcctcacacctgccgagcggaagtgcagtagactgtgcgaggccgaatccagctttaagtacgagagcggcctgttcgtgcagggcctgctgaaagacagcacaggctctttcgtgctcccttttagacaggtgatgtacgccccttaccccaccacacacattgatgtcgacgtgaacaccgtgaaacagatgcctccatgtcacgagcacatctataaccagagaagatacatgcggtccgagctgaccgctttctggcgggccacaagcgaagaggacatggctcaggacacaatcatctacactgatgagtccttcacccctgatctgaacatcttccaagatgtgctgcacagggacaccctggtgaaggccttcctggatcaggtctttcagctgaagcctggcctgtccctgcgctccaccttcctggcccaatttctgctcgtgctgcacagaaaggccctgaccctgattaagtacatcgaggacgatacccagaagggcaagaagcctttcaagtccctgcggaatctgaagatcgacctggacctgaccgccgagggcgatctgaacatcatcatggccctggccgagaagatcaagcccggcctccacagcttcatcttcggcagacctttctacaccagcgtgcaggagagagatgtgctgatgacattttga

SEQ ID NO: 44 - DNA, 1446 bp, synthetic construct
  misc_feature 1..1446: note = Description of Artificial Sequence: Synthetic polynucleotide
  source 1..1446: mol_type = other DNA
atgtctacactgtgtcctccacctagccccgccgtggccaagacagaaatcgccctgagcggaaagtcccctctgctggccgccacatttgcctactgggacaacatactgggccctagagtgcggcacatctgggcccctaagaccgagcaggtgctgctgagcgacggcgagatcaccttcctggccaaccacaccctgaacggcgaaatcctgagaaacgccgaaagcggcgccatcgacgtgaagttcttcgtgctgagcgagaaaggcgtgatcatcgtgtccctgatcttcgacggcaactggaacggcgatagaagcacctacggcctgagcatcattctgcctcagaccgagctgagcttctacctgcctcttcatagagtgtgcgtggacagactgacccacattattagaaagggaagaatctggatgcacaaggaacggcaggagaacgtgcagaaaatcatcctggaagggaccgagcggatggaagatcagggccagagcatcatccccatgctgacaggcgaggtgatccctgtgatggaactgctgtccagcatgaagtctcacagcgtgcccgaggaaatcgatatcgccgatacagtgctgaacgacgatgacatcggcgacagctgccacgagggcttcctgctgaatgccatttctagccacctgcagacatgcggatgtagcgtcgtggtgggctctagcgccgagaaggtgaacaagatcgtgcggaccctgtgcctgttcctgacacctgctgaacgcaagtgcagcagactgtgtgaagccgaaagctcttttaagtacgagagcggcctcttcgtccagggcctgctgaaggacagcaccggctcttttgtgctgcccttcagacaggtgatgtacgccccttaccccaccacccacatcgacgtcgacgtgaataccgtgaaacagatgcctccttgccacgagcacatctacaaccagagaagatacatgagaagcgagctgacagccttctggcgggccacctctgaagaggatatggcccaggacacaatcatctacaccgacgagagcttcacccctgatctgaacatcttccaagacgtgctgcacagagataccctggtgaaggcttttctggaccaggttttccagctgaagcctggactgtctctgagatctaccttccttgctcaatttctgctggtcctccaccggaaagccctgacactgatcaagtacatcgaggacgacacccagaagggcaagaagcccttcaagagcctgaggaacctgaaaatcgacctggatctgaccgccgagggcgacctgaacatcatcatggccctggctgaaaagatcaagcctggcctgcacagtttcatcttcggcagacctttctacaccagcgtgcaggagcgggacgtgctgatgaccttctga

SEQ ID NO: 45 - DNA, 1446 bp, synthetic construct
  misc_feature 1..1446: note = Description of Artificial Sequence: Synthetic polynucleotide
  source 1..1446: mol_type = other DNA
atgagcaccctgtgccccccccccagccccgccgtggccaagaccgagatcgccctgtctggcaagtcccctctgcttgccgctaccttcgcctactgggacaacatcctgggccctagagtgcggcacatctgggcccctaagaccgagcaggtcctgctgagcgacggcgaaatcaccttcctggccaaccacaccctgaacggcgagatcctgcggaacgccgagagcggcgccatcgacgtgaagttcttcgtgctgagcgagaagggcgtgatcatcgtgtccctgatcttcgacggaaattggaacggcgacagatccacatacggcctgagcatcatcctgcctcagacagagctgtccttttacctgcccctgcaccgggtgtgcgtggatagactgacacacatcattagaaagggaagaatctggatgcacaaggaacggcaggagaacgtgcagaaaatcatcctggaaggtacagagagaatggaagatcagggacagtctatcatccccatgctgaccggcgaggtgatccccgtgatggaactgctgagttctatgaagtcccacagcgtgcctgaggaaatcgacatcgccgacaccgtgctgaacgacgatgacataggagatagctgccacgagggcttcctgctgaatgccataagcagccacctgcagacctgtggctgcagcgtcgtggtgggcagcagcgccgaaaaggtgaacaagatcgttagaacactgtgcctgtttctgacccctgctgagcggaagtgcagcagactgtgtgaagccgagtctagcttcaagtacgagtccggcctgttcgtgcaaggcctgctcaaggacagcacaggctccttcgtgctgccttttagacaggtgatgtacgccccttaccccaccacccatatcgacgtggacgtgaacaccgtcaagcagatgcctccatgtcacgagcacatctacaaccagcgtagatacatgagaagcgagcttacagctttctggcgggccacctctgaagaggacatggcccaggacaccatcatctacaccgacgagagcttcacccctgacctgaacatttttcaagatgtgctgcacagagataccctggtgaaagccttcctggatcaggtgttccagctgaaacctggactgagcctgagaagcaccttcttggcacagttcctcctggtcctgcacagaaaggccctgaccctcatcaagtacatcgaggatgatacccagaagggcaaaaagcccttcaagagcctgagaaacctgaagatcgatctggacctgacagccgagggcgacctgaacatcatcatggctctggctgaaaaaatcaagcctggcctgcatagcttcatcttcggcagacctttctatacaagcgtgcaggagcgggacgtgctgatgacattctga

SEQ ID NO: 46 - DNA, 1446 bp, synthetic construct
  misc_feature 1..1446: note = Description of Artificial Sequence: Synthetic polynucleotide
  source 1..1446: mol_type = other DNA
atgagcacactgtgtcctcctccgagccctgctgtggccaagaccgagatcgccctgagcggcaagtccccactcctggctgctacattcgcctactgggacaacatcctgggccctagagtgcggcacatctgggcccccaagacagaacaggttctgctgagtgatggcgagatcaccttcctcgccaatcacaccctgaacggcgaaatcctgagaaacgccgagagcggcgccatcgatgtgaaattcttcgtgctgagcgagaagggcgtgatcatcgtgtccctgatcttcgacggcaactggaacggcgatagaagcacctacggcctgagcatcatcctgccccagaccgagctgagcttctacctgcctctgcaccgggtgtgcgtggacagactgacacacatcattagaaagggcagaatctggatgcacaaggaacggcaggagaacgtgcaaaagatcattctggaagggaccgagcggatggaagatcagggccagagcatcatccctatgctgacaggagaagtgatccccgtgatggaactgctgtctagcatgaaatctcacagcgtgcccgaggaaatcgacatcgccgacaccgtgctgaacgacgacgacatcggcgacagctgccatgagggcttccttctcaacgccatcagcagccacctgcagacctgtggctgcagcgtggtggtcggatcttctgccgaaaaggtgaacaagatcgtgcggaccctgtgcctgttcctgacccctgccgaacggaagtgcagcagactgtgcgaggccgagagcagctttaagtacgagtctggcctgttcgtgcagggcctgctgaaggacagcacaggcagctttgtgctgccttttagacaggtgatgtacgccccttaccccaccacccacatcgacgtcgacgtgaacaccgtgaagcagatgcctccatgtcacgagcacatctacaaccagcggagatacatgagatccgagctgacagccttctggcgggccaccagcgaagaggatatggcccaggatacaatcatctatacagacgagtccttcacccctgatctgaacatctttcaggacgttctgcacagagataccctggtgaaggctttcctggaccaagtgttccagctgaaacctggactgagcctgcggagcacctttctggcccagttcctgctggtcctgcacagaaaggccctgaccctgatcaagtacatcgaggacgatacccagaaaggcaaaaagcctttcaagagcctgagaaatctgaagatcgacctggatctgaccgccgagggagatctgaatatcatcatggccctggccgagaaaatcaagcccggcctccattctttcatcttcggcagacccttctacacatctgtgcaggagcgcgacgtgctgatgaccttctga

SEQ ID NO: 47 - DNA, 1446 bp, synthetic construct
  misc_feature 1..1446: note = Description of Artificial Sequence: Synthetic polynucleotide
  source 1..1446: mol_type = other DNA
atgagcaccctgtgtcctccacccagccctgccgtggccaagacagagatcgccctgtctggaaagagccccctgctggccgctaccttcgcctactgggacaacatcctgggccctagagtgcggcacatctgggcccctaagacagagcaggtcctgctgagcgacggcgaaatcaccttcctggctaatcacacccttaatggagaaatcctgagaaacgccgaatccggcgccatcgacgtgaagttcttcgtgctgagcgagaaaggcgtgatcatcgtgtccctgatctttgatggaaattggaacggcgacagaagcacatacggcctgagcatcatcctgcctcagaccgagctgtctttttacctgcctctgcacagagtgtgcgtggaccggctgacccacatcatcagaaagggcagaatctggatgcacaaggaacggcaggagaacgtgcagaaaatcattctggaaggcaccgagcggatggaagatcagggccagagcatcatccccatgctgaccggcgaggtgatccccgtgatggaactgctgtctagcatgaaatctcactctgtgcctgaggaaatcgacatcgccgacacagtgctgaacgacgacgacatcggcgatagctgccacgagggcttcctgctgaacgccatcagcagccacctgcagacatgcggctgcagcgtggtcgtgggaagcagcgccgaaaaggtgaacaagatcgtgcggaccctctgtctgttcctgacgcccgccgagagaaagtgcagcagactgtgtgaagccgagagcagctttaagtacgagtctggcctgtttgtgcagggcctgctgaaggacagcaccggctctttcgtgctgcccttcagacaggtgatgtacgccccttaccccaccacacacattgacgtggacgtcaacaccgtgaaacagatgcctccttgccatgaacacatctacaaccagcggagatacatgcggagcgagctgaccgccttctggcgggccacctctgaggaagatatggcccaggacaccatcatctatacagacgagtccttcacccctgatctgaatatcttccaagatgttctccacagggacaccctggtgaaggcttttctcgaccaggtgttccagctgaaacctggcctgagcctgcggagcacctttctggcccaatttctgctcgtgctgcacagaaaggccctgaccctgatcaaatacatcgaggacgatacacagaagggcaagaagcctttcaagtccctgagaaacctgaagatcgacctggatctgacagccgagggcgacctgaacatcattatggctctggccgagaagatcaagcctggactccacagcttcatcttcggccgccccttctacaccagcgtgcaagagagagacgtgctgatgaccttctga

SEQ ID NO: 48 - DNA, 1446 bp, synthetic construct
  misc_feature 1..1446: note = Description of Artificial Sequence: Synthetic polynucleotide
  source 1..1446: mol_type = other DNA
atgagcacactgtgcccccccccttctcctgccgtggccaagaccgagattgccctgtccggcaagtcccctctgttggccgccacatttgcctactgggacaacatcctgggccctagagtgcggcacatttgggcccctaagacagaacaggtgctgctgagtgatggcgagatcacctttctggccaaccacaccctgaatggcgaaatcctgagaaacgccgagagcggagccatcgacgtgaagttcttcgtgctgtctgagaagggtgttatcatcgtgtccctgatcttcgacggcaactggaacggcgacagatctacctacggcctttctatcatcctgccccagaccgagctgagcttctacctgcctctgcatcgggtgtgcgtggaccggctgacacacatcattagaaaggggagaatctggatgcacaaggaacgccaggagaacgtgcagaaaatcattctggaagggaccgaaagaatggaagatcagggccagagcatcatccctatgctgacaggagaggtgatccccgtgatggaactgcttagcagcatgaagtctcacagcgtgcccgaggaaatcgacatcgccgacaccgtgctgaacgacgacgatatcggcgactcatgccacgagggcttcctgctgaatgccatcagcagccacctgcagacatgcggctgttctgtggtggtgggctcaagcgccgagaaggtgaacaagatcgtgcggaccctgtgcctgttcctgacacctgctgagcggaagtgcagcagactgtgtgaagccgaatccagctttaagtacgagtctggcctcttcgtgcaaggcctgctgaaggacagcaccggctcttttgtgctgccttttagacaggtgatgtacgccccttaccccaccacacacatcgacgttgatgtcaacaccgtgaaacagatgcctccatgtcacgagcacatctacaaccagagaagatacatgagaagcgagctgaccgccttttggcgggccaccagcgaggaagatatggcccaggacaccatcatctataccgacgagtccttcacccctgatctgaacatcttccaagacgtgctgcaccgggacacactggtcaaggccttcctggaccaagtgttccagctgaagcccggcctgagcctgcggagcaccttcctggctcagttcctgctggtgcttcaccggaaggccctgacccttatcaagtacatcgaggacgacacccagaagggcaaaaagcctttcaagagcctgagaaatctgaaaatcgacctggatctgacagccgaaggcgatctgaacatcatcatggcccttgctgagaaaatcaagccaggcctgcacagctttatcttcggcagacctttctacaccagcgtgcaggagagagatgtgctgatgaccttctga

SEQ ID NO: 49 - DNA, 1446 bp, synthetic construct
  misc_feature 1..1446: note = Description of Artificial Sequence: Synthetic polynucleotide
  source 1..1446: mol_type = other DNA
atgagcaccctctgtcctcctccatctcctgccgtggcaaagaccgagatcgccctgtccggcaaaagccccctgctggccgctacattcgcctactgggacaacatcctcggacctagagtgcggcacatctgggcccctaagaccgagcaggttctgctgagcgacggcgagataacatttctggccaaccacaccctgaacggcgagatcctgagaaacgccgagagcggcgccatcgatgtgaagttcttcgtgctctctgagaagggcgtgatcattgtgtccctgatcttcgacggcaactggaacggcgatagatccacctacggcctgagcatcatcctgccccagacagagctgtctttttacctgcctctgcaccgggtgtgcgtggacagactgacacacatcatcagaaagggcagaatctggatgcacaaggaacggcaggagaacgtgcagaaaatcatcctggaaggcaccgagagaatggaagatcagggccagagcatcattcctatgctgactggagaggtgatccccgtgatggaactgctgtctagcatgaaaagccacagcgtgcccgaggaaatcgacatcgccgacaccgtgctgaacgacgacgacatcggcgacagctgccacgagggcttcctgctcaatgccatcagctcccacctgcagacatgcggctgcagcgtggtcgtgggcagcagcgccgaaaaggtgaacaagatcgtgcggacactgtgtctgttcctgacccctgctgaaagaaagtgcagcagactgtgcgaggccgaatctagctttaagtacgagagcggcctcttcgtgcaaggcctgctgaaggactccacaggcagcttcgtgctgccttttagacaggtgatgtacgccccttatcctacaacccacatcgacgtggacgtcaataccgtgaagcagatgcctccatgtcacgagcacatctacaaccagagaagatacatgagaagcgagctgaccgctttttggcgggccacaagcgaggaagatatggcccaggacaccatcatctatactgatgagtctttcacccctgatctgaacatcttccaagatgtgctccatagagataccctggtcaaagccttcctggaccaggtgttccagctgaaacccggcctgagcctgagatctaccttcctggctcagttcctgctggtgctgcacagaaaggccctgaccctgatcaagtacatcgaggatgatacccagaagggaaaaaagcccttcaagtccctgcggaacctgaagatcgacctggatctgaccgccgagggcgacctgaatatcatcatggccctggccgaaaagatcaagccaggactgcatagcttcatcttcggcagacctttctacacatctgtgcaggagcgggacgtgctgatgaccttctga

SEQ ID NO: 50 - DNA, 1446 bp, synthetic construct
  misc_feature 1..1446: note = Description of Artificial Sequence: Synthetic polynucleotide
  source 1..1446: mol_type = other DNA
atgagcacactctgtcctcctccgagcccagccgtggcaaagaccgagatcgccctgtctggcaagtcccctctgctggccgccaccttcgcctactgggacaacatcctgggccctagagtgcggcacatctgggcccctaagaccgagcaggtgctgctgagcgacggagaaatcaccttcctggctaatcacaccctgaacggcgagatcctgcggaacgccgaaagcggcgccatcgacgtgaagttcttcgtgctgagcgagaagggagtgatcatcgtgtccctgatcttcgacggcaactggaacggcgaccgatctacatacggcctgagcatcatcctgccacagacagagctgagcttttacctgcccctgcatagagtgtgcgtggacagactgacccacatcattagaaagggcagaatctggatgcacaaggaacggcaggagaacgtgcaaaagatcatcctggaaggcaccgaaagaatggaagatcagggccagagcatcattcctatgctgaccggcgaggtgatccccgtgatggaactgttgtccagcatgaaatctcacagcgtccccgaggaaatcgacatcgccgacaccgtgctgaacgacgacgatatcggcgactcatgccatgagggattcctgctgaatgccatcagcagccacctgcagacctgcggctgtagcgtggtcgtgggcagcagtgccgagaaggtgaacaagatcgtgcggaccctgtgtctgtttctgacccctgccgaaagaaagtgcagcagactgtgcgaggccgagagcagcttcaagtacgagtctggcctgttcgtgcagggcctgctgaaagacagcaccggatctttcgtgctgccttttagacaggtgatgtacgccccttatcctacaacccacattgacgtcgacgtcaacaccgtgaaacagatgcctccgtgccacgagcacatctacaaccagaggcggtacatgagatctgagctgacagccttctggcgggccacaagcgaagaggacatggcccaggacaccatcatctacactgatgagagcttcacccctgatctgaacatcttccaagacgtgctgcaccgggacaccctggtcaaggcctttctcgaccaggtgttccagctgaagcccggcctgtccctgagatccacatttcttgctcagttcctgctggtgctgcacagaaaagccctgacactgatcaagtacatcgaggacgacacacagaagggcaaaaagcctttcaaaagcctgagaaacctgaagatcgatctggacctgaccgccgagggcgatcttaatatcatcatggccctggccgaaaaaatcaagcctggcctgcactcttttatcttcggcagacctttctacaccagcgtgcaggagagagatgtgctgatgaccttctga

SEQ ID NO: 51 - DNA, 1446 bp, synthetic construct
  misc_feature 1..1446: note = Description of Artificial Sequence: Synthetic polynucleotide
  source 1..1446: mol_type = other DNA
atgagcaccctctgccccccccccagccccgccgtggccaagacagaaatcgccctgtctggcaagtcccctctgctggccgccacctttgcctactgggacaacatcctgggccctagagtgcggcacatctgggcccctaagaccgagcaagtgctgctgtctgatggagaaatcaccttcctggctaatcacacactgaacggcgagatcctgcggaacgccgagtctggagccatcgacgtgaaattcttcgtgctgagcgagaagggcgtgatcatcgtgtccctgatcttcgacggcaactggaacggcgatagaagcacctacggcctgtccatcatcctgcctcagacagagctgtccttctacctgccactgcaccgggtgtgcgtggacagactgacccacattattagaaagggcagaatctggatgcacaaggaacggcaggagaacgtgcagaagatcattctggaagggaccgagagaatggaagatcagggccagagcatcatccctatgctgactggcgaggtgatccccgtgatggaactgctgagctccatgaaaagccattctgtccccgaggaaatcgacatcgccgacaccgtgctgaacgacgacgatatcggcgacagctgccacgagggcttcctgctgaatgccatcagctctcatctgcagacctgcggctgcagcgtcgtggtgggctctagcgccgagaaggtgaacaagatcgtgcggacactgtgcctgttcctgacacctgccgagaggaagtgcagcagactgtgtgaagccgaatctagctttaagtacgagagcggcctgttcgtgcaaggcctgctgaaggacagcacaggcagcttcgtgctgcctttcagacaggtgatgtacgccccttaccccaccacccacatcgatgttgacgtgaacaccgtgaagcagatgcctccatgtcacgagcacatctacaaccagcggagatacatgcggagcgagctgaccgccttttggcgggccacaagcgaagaggacatggctcaggacacaatcatctacactgatgagagcttcacccctgatctgaacattttccaagacgtgctccacagagataccctggtgaaggccttcctggaccaggttttccagctgaaacctggactgagcctgagaagcaccttcctggcccagttcctgctcgtgctgcacagaaaggccctgacccttatcaagtatatcgaggacgacacccagaaaggcaaaaagcccttcaagagcctgagaaacctgaagatcgacctggatctgaccgccgagggagatctgaacatcatcatggccctggccgagaaaatcaagcctggcctgcacagctttatcttcggccgccccttttacacaagcgtgcaggagagagacgtgctgatgaccttctga

SEQ ID NO: 52 - DNA, 1446 bp, synthetic construct
  misc_feature 1..1446: note = Description of Artificial Sequence: Synthetic polynucleotide
  source 1..1446: mol_type = other DNA
atgagcacactgtgtcctcctcctagccccgccgtggccaagaccgagatcgccctcagcggcaagtctccactgctcgccgctaccttcgcctactgggacaacatcctgggccctagagtgcggcacatctgggcccctaagacagagcaggtccttctgagcgacggcgagataacattcctggccaaccacacactgaacggcgagatcctcaggaacgccgaatctggcgccatcgacgtgaagttcttcgtgctgtctgagaagggcgtgattattgtgtccctgatcttcgacggaaattggaacggcgaccggagcacatacggcctgtccatcatcctgccccagacggaactgtctttttacctgcctctgcacagagtgtgcgtggacagactgacccacatcattagaaagggcagaatctggatgcacaaggaaagacaggagaacgtgcagaaaatcatcctggaaggtacagagagaatggaagatcagggacagagcatcatccctatgctgactggcgaagtgatccccgtgatggaactgctgtccagcatgaaaagccacagcgtgcccgaggaaatcgacatcgccgacactgtgctgaacgacgatgatatcggcgacagctgccatgagggcttcctgctgaatgccatcagctctcacctgcagacctgtggatgtagcgtggtggtcggcagcagcgccgaaaaggtgaacaagattgtgcggaccctgtgcctgttcctcacacctgctgagagaaagtgcagcagactgtgcgaggccgagagcagcttcaagtacgagagcggcctgttcgtgcagggcctgctgaaggacagcaccggctccttcgttctgcctttccggcaggtgatgtacgccccttaccccaccacccacatcgatgttgacgtgaataccgtgaaacagatgcctccatgtcacgagcacatctacaaccagagaagatacatgagaagcgagctgaccgccttctggcgggccaccagcgaagaggacatggcccaggacaccatcatctacaccgacgagagcttcacccctgatctgaacatctttcaggatgtgctccatagagataccctggtcaaggccttcctggaccaggtgttccagctgaaacctggactgagcctgcgcagcaccttcctggctcaatttctacttgtgctgcaccggaaggccctgacactgatcaagtacatcgaggacgacacccagaagggcaaaaagccctttaagagcctgagaaacctgaagatcgacctggatctgacagccgaaggcgatctgaacatcatcatggctcttgctgagaaaatcaagccaggactgcattctttcatcttcggccgccccttctacacatctgtgcaggagcgggacgtgctgatgaccttctga

SEQ ID NO: 53 - DNA, 8506 bp, synthetic construct
  misc_feature 1..8506: note = Description of Artificial Sequence: Synthetic polynucleotide
  source 1..8506: mol_type = other DNA
agtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggcacccaacttttctatacaaagttgtaatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcaccaccaccaccaccacgagcagaagctgatctccgaggaggacctgtaaatcaaggttacaagacaggaataaatttaaggagaccaatagaaactgggcttgtcgagacagagaagactcttgcgtttctgataggcacctattggtcttactgacatccactttgcctttctctccacagaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactacccctacgacgtgcccgactacgcctaaacaactttgtataataaagttgtaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagt

SEQ ID NO: 54 - DNA, 1195 bp, synthetic construct
  misc_feature 1..1195: note = Description of Artificial Sequence: Synthetic polynucleotide
  misc_difference 1..11: note = modified_base - a, c, t, g, unknown or other
  misc_difference 13..16: note = modified_base - a, c, t, g, unknown or other
  misc_difference 22: note = modified_base - a, c, t, g, unknown or other
  misc_difference 912: note = modified_base - a, c, t, g, unknown or other
  misc_difference 925: note = modified_base - a, c, t, g, unknown or other
  misc_difference 964: note = modified_base - a, c, t, g, unknown or other
  misc_difference 968: note = modified_base - a, c, t, g, unknown or other
  misc_difference 970: note = modified_base - a, c, t, g, unknown or other
  misc_difference 974: note = modified_base - a, c, t, g, unknown or other
  misc_difference 977..978: note = modified_base - a, c, t, g, unknown or other
  misc_difference 980..982: note = modified_base - a, c, t, g, unknown or other
  misc_difference 988..989: note = modified_base - a, c, t, g, unknown or other
  misc_difference 999: note = modified_base - a, c, t, g, unknown or other
  misc_difference 1013: note = modified_base - a, c, t, g, unknown or other
  misc_difference 1018: note = modified_base - a, c, t, g, unknown or other
  misc_difference 1021..1022: note = modified_base - a, c, t, g, unknown or other
  misc_difference 1024..1026: note = modified_base - a, c, t, g, unknown or other
  misc_difference 1033: note = modified_base - a, c, t, g, unknown or other
  misc_difference 1038: note = modified_base - a, c, t, g, unknown or other
  misc_difference 1041..1045: note = modified_base - a, c, t, g, unknown or other
  misc_difference 1047..1049: note = modified_base - a, c, t, g, unknown or other
  misc_difference 1058..1064: note = modified_base - a, c, t, g, unknown or other
  misc_difference 1066: note = modified_base - a, c, t, g, unknown or other
  misc_difference 1068..1070: note = modified_base - a, c, t, g, unknown or other
  misc_difference 1072: note = modified_base - a, c, t, g, unknown or other
  misc_difference 1077: note = modified_base - a, c, t, g, unknown or other
  misc_difference 1082..1085: note = modified_base - a, c, t, g, unknown or other
  misc_difference 1087: note = modified_base - a, c, t, g, unknown or other
  misc_difference 1090..1095: note = modified_base - a, c, t, g, unknown or other
  misc_difference 1100..1102: note = modified_base - a, c, t, g, unknown or other
  misc_difference 1104..1105: note = modified_base - a, c, t, g, unknown or other
  misc_difference 1108: note = modified_base - a, c, t, g, unknown or other
  misc_difference 1111..1112: note = modified_base - a, c, t, g, unknown or other
  misc_difference 1114: note = modified_base - a, c, t, g, unknown or other
  misc_difference 1118..1120: note = modified_base - a, c, t, g, unknown or other
  misc_difference 1123..1126: note = modified_base - a, c, t, g, unknown or other
  misc_difference 1129..1134: note = modified_base - a, c, t, g, unknown or other
  misc_difference 1136..1137: note = modified_base - a, c, t, g, unknown or other
  misc_difference 1140: note = modified_base - a, c, t, g, unknown or other
  misc_difference 1142..1191: note = modified_base - a, c, t, g, unknown or other
  misc_difference 1193: note = modified_base - a, c, t, g, unknown or other
  misc_difference 1195: note = modified_base - a, c, t, g, unknown or other
  source 1..1195: mol_type = other DNA
nnnnnnnnnnncnnnntgttcntgccttcttctttttcctacagctcctgggcaacgccaccatggcacccaacttttctatacaaagttgtaatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcaccaccaccaccaccacgagcagaagctgatctccgaggaggacctgtaaatcaaggttacaagacaggaataaatttaaggagaccaatagaaactgggcttgtcgagacagagaagactcttgcgtttctgataggcacctattggncttactgacatcnctttgcctttctctcacagaatgcatcagctcacacttncaancngtgntgnncnnntagtannagcagtgcanagaagtaaataganagtcnganntnnnctttttnctgantcnnnnnannnaaatgctcnnnnnnnancnnnancatcntttannnnantcnnnnnnttgtnnngnngcnaanntnactnnnctnnnnctnnnnnnanncangnnnnnnnnnnnnnnnnnnnnnnnnnnnnnnnnnnnnnnnnnnnnnnnnnngncn

SEQ ID NO: 55 - DNA, 1212 bp, synthetic construct
  misc_feature 1..1212: note = Description of Artificial Sequence: Synthetic polynucleotide
  misc_difference 1..10: note = modified_base - a, c, t, g, unknown or other
  misc_difference 14: note = modified_base - a, c, t, g, unknown or other
  misc_difference 832..833: note = modified_base - a, c, t, g, unknown or other
  misc_difference 851: note = modified_base - a, c, t, g, unknown or other
  misc_difference 920: note = modified_base - a, c, t, g, unknown or other
  misc_difference 978: note = modified_base - a, c, t, g, unknown or other
  misc_difference 987: note = modified_base - a, c, t, g, unknown or other
  misc_difference 992: note = modified_base - a, c, t, g, unknown or other
  misc_difference 996: note = modified_base - a, c, t, g, unknown or other
  misc_difference 1005: note = modified_base - a, c, t, g, unknown or other
  misc_difference 1013: note = modified_base - a, c, t, g, unknown or other
  misc_difference 1025..1027: note = modified_base - a, c, t, g, unknown or other
  misc_difference 1031: note = modified_base - a, c, t, g, unknown or other
  misc_difference 1037: note = modified_base - a, c, t, g, unknown or other
  misc_difference 1043..1053: note = modified_base - a, c, t, g, unknown or other
  misc_difference 1058: note = modified_base - a, c, t, g, unknown or other
  misc_difference 1061: note = modified_base - a, c, t, g, unknown or other
  misc_difference 1065..1070: note = modified_base - a, c, t, g, unknown or other
  misc_difference 1072: note = modified_base - a, c, t, g, unknown or other
  misc_difference 1077: note = modified_base - a, c, t, g, unknown or other
  misc_difference 1080..1081: note = modified_base - a, c, t, g, unknown or other
  misc_difference 1083..1084: note = modified_base - a, c, t, g, unknown or other
  misc_difference 1089: note = modified_base - a, c, t, g, unknown or other
  misc_difference 1092..1093: note = modified_base - a, c, t, g, unknown or other
  misc_difference 1096..1097: note = modified_base - a, c, t, g, unknown or other
  misc_difference 1099: note = modified_base - a, c, t, g, unknown or other
  misc_difference 1101: note = modified_base - a, c, t, g, unknown or other
  misc_difference 1103..1105: note = modified_base - a, c, t, g, unknown or other
  misc_difference 1108..1130: note = modified_base - a, c, t, g, unknown or other
  misc_difference 1132: note = modified_base - a, c, t, g, unknown or other
  misc_difference 1134: note = modified_base - a, c, t, g, unknown or other
  misc_difference 1136: note = modified_base - a, c, t, g, unknown or other
  misc_difference 1139..1140: note = modified_base - a, c, t, g, unknown or other
  misc_difference 1146..1147: note = modified_base - a, c, t, g, unknown or other
  misc_difference 1149..1156: note = modified_base - a, c, t, g, unknown or other
  misc_difference 1158..1159: note = modified_base - a, c, t, g, unknown or other
  misc_difference 1161..1167: note = modified_base - a, c, t, g, unknown or other
  misc_difference 1169..1170: note = modified_base - a, c, t, g, unknown or other
  misc_difference 1172..1176: note = modified_base - a, c, t, g, unknown or other
  misc_difference 1179: note = modified_base - a, c, t, g, unknown or other
  misc_difference 1182: note = modified_base - a, c, t, g, unknown or other
  misc_difference 1185: note = modified_base - a, c, t, g, unknown or other
  misc_difference 1187..1189: note = modified_base - a, c, t, g, unknown or other
  misc_difference 1192..1209: note = modified_base - a, c, t, g, unknown or other
  misc_difference 1211: note = modified_base - a, c, t, g, unknown or other
  source 1..1212: mol_type = other DNA
nnnnnnnnnnattnagcagcgtatccacatagcgtaaaggagcaacatagttaagaataccagtcaatctttcacaaattttgtaatccagaggttgatttacaactttattatacaaagttgtttacaggtcctcctcggagatcagcttctgctcgtggtggtggtggtggtgaaaagtcattagaacatctcgttcttgcacactagtgtagaaaggtcttccaaagataaaagagtgtaggcctggtttaattttctcagccagagccattattatgttaagatcgccctctgctgttaaatcaaggtctatcttcaggttccgaagagatttaaagggcttttttcccttctgcgtatcgtcttctatatattttattagtgtcaaggcttttctgtgaaggacaagtagaaactgtgcaaggaaagtacttctgagagataagccaggtttcagctgaaagacctgatccaggaaggctttcactagagtgtctctgtgtaagacatcttgaaaaatattcaaatcaggagtaaagctttcgtcagtgtagatgatcgtatcctgagccatgtcttcttctgaagtggctctccagaaggctgtcagctcggatctcatgtatctacgctgattataaatatgttcatgacagggtggcatctgcttcacagtattgacatccacatctatgtgtgtggtgggatatggagcatacatgacttgccggaaaggcagcacaaagcttccagttgaatcctttagcaggccttgtacaaagagccctgactcatatttaaatgatgattctgcttcacataacctggnncattttctctctgctggngtcagaaaaaggcataatgttctgactatcttatttactttctctgcactgctacctactacaacgganagccacaggtttgcaagtgtgagctgatggcattctgtggagagaaaggcaaagtggntgtcagtanaccantagngcctatcanaaacgcanagtcttctctgnnncganagccantttctnnnnnnnnnnnaattnttnctgnnnnnnanctganttnncnngtccnccnncgnnanantnnnctnnnnnnnnnnnnnnnnnnnnnnntncnanaannaaagcnncnnnnnnnncnntnnnnnnncnncnnnnntgnagnacngnnntcnnnnnnnnnnnnnnnnnngna

SEQ ID NO: 56 - DNA, 7881 bp, synthetic construct
  misc_feature 1..7881: note = Description of Artificial Sequence: Synthetic polynucleotide
  source 1..7881: mol_type = other DNA
agtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttaggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacagacgcgtctcgaacgccaccatggcacccaacttttctatacaaagttgtaatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcaccaccaccaccaccacgagcagaagctgatctccgaggaggacctgtaaatcaaggttacaagacaggaataaatttaaggagaccaatagaaactgggcttgtcgagacagagaagactcttgcgtttctgataggcacctattggtcttactgacatccactttgcctttctctccacagaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactacccctacgacgtgcccgactacgcctaaacaactttgtataataaagttgtaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagt

SEQ ID NO: 57 - DNA, 1164 bp, synthetic construct
  misc_feature 1..1164: note = Description of Artificial Sequence: Synthetic polynucleotide
  misc_difference 1..10: note = modified_base - a, c, t, g, unknown or other
  misc_difference 832: note = modified_base - a, c, t, g, unknown or other
  misc_difference 919: note = modified_base - a, c, t, g, unknown or other
  misc_difference 923: note = modified_base - a, c, t, g, unknown or other
  misc_difference 957: note = modified_base - a, c, t, g, unknown or other
  misc_difference 963: note = modified_base - a, c, t, g, unknown or other
  misc_difference 965: note = modified_base - a, c, t, g, unknown or other
  misc_difference 970..972: note = modified_base - a, c, t, g, unknown or other
  misc_difference 980..985: note = modified_base - a, c, t, g, unknown or other
  misc_difference 987..988: note = modified_base - a, c, t, g, unknown or other
  misc_difference 990: note = modified_base - a, c, t, g, unknown or other
  misc_difference 995..999: note = modified_base - a, c, t, g, unknown or other
  misc_difference 1003: note = modified_base - a, c, t, g, unknown or other
  misc_difference 1013..1014: note = modified_base - a, c, t, g, unknown or other
  misc_difference 1016: note = modified_base - a, c, t, g, unknown or other
  misc_difference 1019: note = modified_base - a, c, t, g, unknown or other
  misc_difference 1021: note = modified_base - a, c, t, g, unknown or other
  misc_difference 1024..1025: note = modified_base - a, c, t, g, unknown or other
  misc_difference 1028: note = modified_base - a, c, t, g, unknown or other
  misc_difference 1030..1031: note = modified_base - a, c, t, g, unknown or other
  misc_difference 1033..1039: note = modified_base - a, c, t, g, unknown or other
  misc_difference 1042..1046: note = modified_base - a, c, t, g, unknown or other
  misc_difference 1052: note = modified_base - a, c, t, g, unknown or other
  misc_difference 1060..1062: note = modified_base - a, c, t, g, unknown or other
  misc_difference 1064: note = modified_base - a, c, t, g, unknown or other
  misc_difference 1066..1067: note = modified_base - a, c, t, g, unknown or other
  misc_difference 1069..1071: note = modified_base - a, c, t, g, unknown or other
  misc_difference 1073..1076: note = modified_base - a, c, t, g, unknown or other
  misc_difference 1083: note = modified_base - a, c, t, g, unknown or other
  misc_difference 1085: note = modified_base - a, c, t, g, unknown or other
  misc_difference 1087..1091: note = modified_base - a, c, t, g, unknown or other
  misc_difference 1093..1095: note = modified_base - a, c, t, g, unknown or other
  misc_difference 1097..1098: note = modified_base - a, c, t, g, unknown or other
  misc_difference 1100..1106: note = modified_base - a, c, t, g, unknown or other
  misc_difference 1108: note = modified_base - a, c, t, g, unknown or other
  misc_difference 1110: note = modified_base - a, c, t, g, unknown or other
  misc_difference 1112: note = modified_base - a, c, t, g, unknown or other
  misc_difference 1114..1116: note = modified_base - a, c, t, g, unknown or other
  misc_difference 1119..1120: note = modified_base - a, c, t, g, unknown or other
  misc_difference 1122: note = modified_base - a, c, t, g, unknown or other
  misc_difference 1124..1126: note = modified_base - a, c, t, g, unknown or other
  misc_difference 1128..1130: note = modified_base - a, c, t, g, unknown or other
  misc_difference 1132..1133: note = modified_base - a, c, t, g, unknown or other
  misc_difference 1135..1137: note = modified_base - a, c, t, g, unknown or other
  misc_difference 1139..1142: note = modified_base - a, c, t, g, unknown or other
  misc_difference 1147: note = modified_base - a, c, t, g, unknown or other
  misc_difference 1149..1156: note = modified_base - a, c, t, g, unknown or other
  misc_difference 1158..1159: note = modified_base - a, c, t, g, unknown or other
  misc_difference 1162..1164: note = modified_base - a, c, t, g, unknown or other
  source 1..1164: mol_type = other DNA
nnnnnnnnnnattaagcagcgtatccacatagcgtaaaggagcaacatagttaagaataccagtcaatctttcacaaattttgtaatccagaggttgatttacaactttattatacaaagttgtttacaggtcctcctcggagatcagcttctgctcgtggtggtggtggtggtgaaaagtcattagaacatctcgttcttgcacactagtgtagaaaggtcttccaaagataaaagagtgtaggcctggtttaattttctcagccagagccattattatgttaagatcgccctctgctgttaaatcaaggtctatcttcaggttccgaagagatttaaagggcttttttcccttctgcgtatcgtcttctatatattttattagtgtcaaggcttttctgtgaaggacaagtagaaactgtgcaaggaaagtacttctgagagataagccaggtttcagctgaaagacctgatccaggaaggctttcactagagtgtctctgtgtaagacatcttgaaaaatattcaaatcaggagtaaagctttcgtcagtgtagatgatcgtatcctgagccatgtcttcttctgaagtggctctccagaaggctgtcagctcggatctcatgtatctacgctgattataaatatgttcatgacagggtggcatctgcttcacagtattgacatccacatctatgtgtgtggtgggatatggagcatacatgacttgccggaaaggcagcacaaagcttccagttgaatcctttagcaggccttgtacaaagagccctgactcatatttaaatgatgattctgcttcacataacctggngcattttctctctgctggagtcagaaaaaggcataatgttctgactatcttatttactttctctgcactgctacctactacacgganagcncaggtttgcagtgtgagctgatggcattctgtgngagaangnaagtnnngtcagtannnnnngnncnatcannnnnagantcttctctgnntngananccnnttncnntnnnnnnnaannnnngtctgnactgattnnngncnncnnngnnnntcagctncngnnnnngnnngnngnnnnnnntncnanannnaanncntnnngnnncnntnnncnnnntcatncnnnnnnnnannacnnn

SEQ ID NO: 58 - DNA, 1162 bp, synthetic construct
  misc_feature 1..1162: note = Description of Artificial Sequence: Synthetic polynucleotide
  misc_difference 1..12: note = modified_base - a, c, t, g, unknown or other
  misc_difference 16..18: note = modified_base - a, c, t, g, unknown or other
  misc_difference 920: note = modified_base - a, c, t, g, unknown or other
  misc_difference 923: note = modified_base - a, c, t, g, unknown or other
  misc_difference 925: note = modified_base - a, c, t, g, unknown or other
  misc_difference 948: note = modified_base - a, c, t, g, unknown or other
  misc_difference 955..956: note = modified_base - a, c, t, g, unknown or other
  misc_difference 958: note = modified_base - a, c, t, g, unknown or other
  misc_difference 984: note = modified_base - a, c, t, g, unknown or other
  misc_difference 989: note = modified_base - a, c, t, g, unknown or other
  misc_difference 992: note = modified_base - a, c, t, g, unknown or other
  misc_difference 1005..1006: note = modified_base - a, c, t, g, unknown or other
  misc_difference 1009: note = modified_base - a, c, t, g, unknown or other
  misc_difference 1013: note = modified_base - a, c, t, g, unknown or other
  misc_difference 1015: note = modified_base - a, c, t, g, unknown or other
  misc_difference 1017..1023: note = modified_base - a, c, t, g, unknown or other
  misc_difference 1029: note = modified_base - a, c, t, g, unknown or other
  misc_difference 1038..1039: note = modified_base - a, c, t, g, unknown or other
  misc_difference 1041: note = modified_base - a, c, t, g, unknown or other
  misc_difference 1048..1049: note = modified_base - a, c, t, g, unknown or other
  misc_difference 1053: note = modified_base - a, c, t, g, unknown or other
  misc_difference 1058..1059: note = modified_base - a, c, t, g, unknown or other
  misc_difference 1061..1062: note = modified_base - a, c, t, g, unknown or other
  misc_difference 1064..1066: note = modified_base - a, c, t, g, unknown or other
  misc_difference 1068: note = modified_base - a, c, t, g, unknown or other
  misc_difference 1073: note = modified_base - a, c, t, g, unknown or other
  misc_difference 1080: note = modified_base - a, c, t, g, unknown or other
  misc_difference 1082..1083: note = modified_base - a, c, t, g, unknown or other
  misc_difference 1085..1090: note = modified_base - a, c, t, g, unknown or other
  misc_difference 1098..1101: note = modified_base - a, c, t, g, unknown or other
  misc_difference 1103..1106: note = modified_base - a, c, t, g, unknown or other
  misc_difference 1110..1111: note = modified_base - a, c, t, g, unknown or other
  misc_difference 1113: note = modified_base - a, c, t, g, unknown or other
  misc_difference 1115: note = modified_base - a, c, t, g, unknown or other
  misc_difference 1118..1125: note = modified_base - a, c, t, g, unknown or other
  misc_difference 1127: note = modified_base - a, c, t, g, unknown or other
  misc_difference 1130..1135: note = modified_base - a, c, t, g, unknown or other
  misc_difference 1138..1139: note = modified_base - a, c, t, g, unknown or other
  misc_difference 1142..1147: note = modified_base - a, c, t, g, unknown or other
  misc_difference 1153: note = modified_base - a, c, t, g, unknown or other
  misc_difference 1155: note = modified_base - a, c, t, g, unknown or other
  misc_difference 1157: note = modified_base - a, c, t, g, unknown or other
  misc_difference 1159..1162: note = modified_base - a, c, t, g, unknown or other
  source 1..1162: mol_type = other DNA
nnnnnnnnnnnnggtnnngccgatgatgcctctactaaccatgttcatgttttctttttttttctacaggtcctgggtgacgaacagacgcgtctcgaacgccaccatggcacccaacttttctatacaaagttgtaatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcaccaccaccaccaccacgagcagaagctgatctccgaggaggacctgtaaatcaagggttacaagacaggaataaatttaaggagaccaatagaaactgggcttgtcgagacngananactcttgcgtttctgataggcanctattgnntnctgacatccactttgcctttctctcncagangcntcagctcacactnnaanctgngntnnnnnnnagtagnagcagtgcnnanaagtaannaganagtcnnanntnnncnttttnctgactncnncnnnnnnaatgctcnnnnannnnaagnnancntcnnnnnnnnantcnnnnnnttnnacnnnnnnctaaangnantnnnn

SEQ ID NO: 59 - DNA, 8806 bp, synthetic construct
  misc_feature 1..8806: note = Description of Artificial Sequence: Synthetic polynucleotide
  source 1..8806: mol_type = other DNA
agtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggacaactttgtatacaaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgaccaccaccaccaccaccacgagcagaagctgatctccgaggaggacctgtaacacccaacttttctatacaaagttgtagtatccaaggtagtggactagtgtgacgctgctgacccctttctttcccttctgcagaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactacccctacgacgtgcccgactacgcctaaacaactttgtataataaagttgtagccttgataacttcgtataatgtatgctatacgaagttatccgaatcgcaataacttcgtataaagtatcctatacgaagttatcgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagt

SEQ ID NO: 60 - DNA, 1153 bp, synthetic construct
  misc_feature 1..1153: note = Description of Artificial Sequence: Synthetic polynucleotide
  misc_difference 1..22: note = modified_base - a, c, t, g, unknown or other
  misc_difference 28: note = modified_base - a, c, t, g, unknown or other
  misc_difference 867: note = modified_base - a, c, t, g, unknown or other
  misc_difference 908: note = modified_base - a, c, t, g, unknown or other
  misc_difference 917: note = modified_base - a, c, t, g, unknown or other
  misc_difference 921: note = modified_base - a, c, t, g, unknown or other
  misc_difference 992: note = modified_base - a, c, t, g, unknown or other
  misc_difference 1006..1008: note = modified_base - a, c, t, g, unknown or other
  misc_difference 1013: note = modified_base - a, c, t, g, unknown or other
  misc_difference 1018: note = modified_base - a, c, t, g, unknown or other
  misc_difference 1020: note = modified_base - a, c, t, g, unknown or other
  misc_difference 1023: note = modified_base - a, c, t, g, unknown or other
  misc_difference 1036: note = modified_base - a, c, t, g, unknown or other
  misc_difference 1038..1039: note = modified_base - a, c, t, g, unknown or other
  misc_difference 1041: note = modified_base - a, c, t, g, unknown or other
  misc_difference 1043: note = modified_base - a, c, t, g, unknown or other
  misc_difference 1045..1046: note = modified_base - a, c, t, g, unknown or other
  misc_difference 1067: note = modified_base - a, c, t, g, unknown or other
  misc_difference 1070: note = modified_base - a, c, t, g, unknown or other
  misc_difference 1072: note = modified_base - a, c, t, g, unknown or other
  misc_difference 1084: note = modified_base - a, c, t, g, unknown or other
  misc_difference 1086..1091: note = modified_base - a, c, t, g, unknown or other
  misc_difference 1094: note = modified_base - a, c, t, g, unknown or other
  misc_difference 1098..1100: note = modified_base - a, c, t, g, unknown or other
  misc_difference 1102: note = modified_base - a, c, t, g, unknown or other
  misc_difference 1108: note = modified_base - a, c, t, g, unknown or other
  misc_difference 1117..1118: note = modified_base - a, c, t, g, unknown or other
  misc_difference 1123..1126: note = modified_base - a, c, t, g, unknown or other
  misc_difference 1130..1132: note = modified_base - a, c, t, g, unknown or other
  misc_difference 1136: note = modified_base - a, c, t, g, unknown or other
  misc_difference 1138..1144: note = modified_base - a, c, t, g, unknown or other
  misc_difference 1146: note = modified_base - a, c, t, g, unknown or other
  misc_difference 1148..1151: note = modified_base - a, c, t, g, unknown or other
  misc_difference 1153: note = modified_base - a, c, t, g, unknown or other
  source 1..1153: mol_type = other DNA
nnnnnnnnnnnnnnnnnnnnnntgttcntgccttcttctttttcctacagctcctgggcaacgccaccatggacaactttgtatacaaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgaccaccaccaccaccaccacgagcagaagctgatctccgaggagganctgtaacacccaacttttctatacaaagttgtagtatccanggtagtggnctantgtgacgctgctgacccctttctttcccttctgcagaatgccatcagctcacacttgcaaacctgtggctngttccgttgtagtnnnagcantgcananaantaaataagatagncnnancntnntgcctttttctgactcagcanaananaaaatgctccangnnnnnntgnagcnnnancattcntttaaaatnntgagnnnnggcnnntttngnnnnnnnangnnnngn

SEQ ID NO: 61 - DNA, 645 bp, synthetic construct
  misc_feature 1..645: note = Description of Artificial Sequence: Synthetic polynucleotide
  misc_difference 1..17: note = modified_base - a, c, t, g, unknown or other
  misc_difference 19..22: note = modified_base - a, c, t, g, unknown or other
  misc_difference 645: note = modified_base - a, c, t, g, unknown or other
  source 1..645: mol_type = other DNA
nnnnnnnnnnnnnnnnntnnnncagcgtatccacatagcgtaaaaggagcaacatagttaagaataccagtcaatctttcacaaattttgtaatccagaggttgatttcgataacttcgtataggatactttatacgaagttattgcgattcggataacttcgtatagcatacattatacgaagttatcaaggctacaactttattatacaaagttgtttaggcgtagtcgggcacgtcgtaggggtagtggtggtggtggtggtgaaaagtcattataacatctcgttcttgcacactagtgtagaaaggtcttccaaagataaaagagtgtaggcctggtttaattttctcagccagagccattattatgttaagatcgccctctgctgttaaatcaaggtctatcttcaggttccgaagagatttaaagggcttttttcccttctgcgtatcgtcttctatatattttattagtgtcaaggcttttctgtgaaggacaagtagaaactgtgcaaggaaagtacttctgagagataagccaggtttcagctgaaagacctgatccaggaaggctttcactagagtgtctctgtgtaaaacatcttgaaaaatattccaatcaggagtatagctttcgtcagtn

SEQ ID NO: 62 - DNA, 8755 bp, synthetic construct
  misc_feature 1..8755: note = Description of Artificial Sequence: Synthetic polynucleotide
  source 1..8755: mol_type = other DNA
agtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggacaactttgtatacaaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagttgactcgttggatccccactacagccgatactcaagcttgacgaattcgaccacccaacttttctatacaaagttgtagtatccaaggtagtggactagtgtgacgctgctgacccctttctttcccttctgcagaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactacccctacgacgtgcccgactacgcctaaacaactttgtataataaagttgtagccttgataacttcgtataatgtatgctatacgaagttatccgaatcgcaataacttcgtataaagtatcctatacgaagttatcgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagt

SEQ ID NO: 63 - DNA, 1079 bp, synthetic construct
  misc_feature 1..1079: note = Description of Artificial Sequence: Synthetic polynucleotide
  misc_difference 1..18: note = modified_base - a, c, t, g, unknown or other
  misc_difference 20..23: note = modified_base - a, c, t, g, unknown or other
  misc_difference 29: note = modified_base - a, c, t, g, unknown or other
  misc_difference 812: note = modified_base - a, c, t, g, unknown or other
  misc_difference 858: note = modified_base - a, c, t, g, unknown or other
  misc_difference 905: note = modified_base - a, c, t, g, unknown or other
  misc_difference 946: note = modified_base - a, c, t, g, unknown or other
  misc_difference 959..960: note = modified_base - a, c, t, g, unknown or other
  misc_difference 972: note = modified_base - a, c, t, g, unknown or other
  misc_difference 975..977: note = modified_base - a, c, t, g, unknown or other
  misc_difference 982..983: note = modified_base - a, c, t, g, unknown or other
  misc_difference 985: note = modified_base - a, c, t, g, unknown or other
  misc_difference 990..991: note = modified_base - a, c, t, g, unknown or other
  misc_difference 1019: note = modified_base - a, c, t, g, unknown or other
  misc_difference 1022: note = modified_base - a, c, t, g, unknown or other
  misc_difference 1024: note = modified_base - a, c, t, g, unknown or other
  misc_difference 1049: note = modified_base - a, c, t, g, unknown or other
  misc_difference 1052: note = modified_base - a, c, t, g, unknown or other
  misc_difference 1072..1079: note = modified_base - a, c, t, g, unknown or other
  source 1..1079: mol_type = other DNA
nnnnnnnnnnnnnnnnnncnnnntgttcntgccttcttctttttcctacagctcctgggcaacgccaccatggacaactttgtatacaaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagttgactcgttggatccccactacagccgatactcaagcttngacgaattcgaccacccaacttttctatacaaagttgtagtatccnaaggtagtggactagtgtgacgctgctgacccctttctttcccttcntgcagaatgccatcagctcacacttgcaaacctgtggctgntccgttgtagtannagcagtgcaganaannnaatannanagtcnnaacattatgccttttctgactccagcanaananaaaatgctccaggttatgtgaagcnaantcatcatttaaatatgagtnnnnnnnn

SEQ ID NO: 64 - DNA, 1058 bp, synthetic construct
  misc_feature 1..1058: note = Description of Artificial Sequence: Synthetic polynucleotide
  misc_difference 1..13: note = modified_base - a, c, t, g, unknown or other
  misc_difference 15..16: note = modified_base - a, c, t, g, unknown or other
  misc_difference 18..23: note = modified_base - a, c, t, g, unknown or other
  misc_difference 34: note = modified_base - a, c, t, g, unknown or other
  misc_difference 83: note = modified_base - a, c, t, g, unknown or other
  misc_difference 798: note = modified_base - a, c, t, g, unknown or other
  misc_difference 865..866: note = modified_base - a, c, t, g, unknown or other
  misc_difference 939..940: note = modified_base - a, c, t, g, unknown or other
  misc_difference 961: note = modified_base - a, c, t, g, unknown or other
  misc_difference 971: note = modified_base - a, c, t, g, unknown or other
  misc_difference 976: note = modified_base - a, c, t, g, unknown or other
  misc_difference 983: note = modified_base - a, c, t, g, unknown or other
  misc_difference 1000: note = modified_base - a, c, t, g, unknown or other
  misc_difference 1015..1016: note = modified_base - a, c, t, g, unknown or other
  misc_difference 1018: note = modified_base - a, c, t, g, unknown or other
  misc_difference 1020: note = modified_base - a, c, t, g, unknown or other
  misc_difference 1023..1024: note = modified_base - a, c, t, g, unknown or other
  misc_difference 1032: note = modified_base - a, c, t, g, unknown or other
  misc_difference 1047: note = modified_base - a, c, t, g, unknown or other
  misc_difference 1049..1050: note = modified_base - a, c, t, g, unknown or other
  misc_difference 1056: note = modified_base - a, c, t, g, unknown or other
  source 1..1058: mol_type = other DNA
nnnnnnnnnnnnngnntnnnnnncagcgtatccncatagcgtaaaaggagcaacatagttaagaataccagtcaatctttcanaaattttgtaatccagaggttgatttcgataacttcgtataggatactttatacgaagttattgcgattcggataacttcgtatagcatacattatacgaagttatcaaggctacaactttattatacaaagttgtttaggcgtagtcgggcacgtcgtaggggtagtggtggtggtggtggtgaaaagtcattagaacatctcgttcttgcacactagtgtagaaaggtcttccaaagataaaagagtgtaggcctggtttaattttctcagccagagccattattatgttaagatcgccctctgctgttaaatcaaggtctatcttcaggttccgaagagatttaaagggcttttttcccttctgcgtatcgtcttctatatattttattagtgtcaaggcttttctgtgaaggacaagtagaaactgtgcaaggaaagtacttctgagagataagccaggtttcagctgaaagacctgatccaggaaggctttcactagagtgtctctgtgtaagacatcttgaaaaatattcaaatcaggagtaaagctttcgtcagtgtagatgatcgtatcctgagccatgtcttcttctgaagtggctctccagaaggctgtcagctcggatctcatgtatctacgctgattataaatatgttcatgacagggtggcatctgcttcacagtattgacatccacatctatgtgtgtggngggatatggagcatacatgactttgccggaaaggcagcacaaagcttccagttgaatccttttagcnnccttgtacaaagagccctgactcatattttaaatgatgattctgcttcacataacctggagcattttctctcnngctgggagtcagaaaagggcntaatgttctngactnatcttanttactttctctgcaccngcctacctactacanngnancannccacaggntttgcaagtggtgancnnatggcnat

SEQ ID NO: 65 - DNA, 8759 bp, synthetic construct
  misc_feature 1..8759: note = Description of Artificial Sequence: Synthetic polynucleotide
  source 1..8759: mol_type = other DNA
agtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggacaactttgtatacaaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgactgaccacccaacttttctatacaaagttgtagtatccaaggtagtggactagtgtgacgctgctgacccctttctttcccttctgcagaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactacccctacgacgtgcccgactacgcctaaacaactttgtataataaagttgtagccttgataacttcgtataatgtatgctatacgaagttatccgaatcgcaataacttcgtataaagtatcctatacgaagttatcgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagt

SEQ ID NO: 66 - DNA, 775 bp, synthetic construct
  misc_feature 1..775: note = Description of Artificial Sequence: Synthetic polynucleotide
  misc_difference 1..22: note = modified_base - a, c, t, g, unknown or other
  misc_difference 25: note = modified_base - a, c, t, g, unknown or other
  misc_difference 43: note = modified_base - a, c, t, g, unknown or other
  misc_difference 739..740: note = modified_base - a, c, t, g, unknown or other
  misc_difference 767..768: note = modified_base - a, c, t, g, unknown or other
  misc_difference 771: note = modified_base - a, c, t, g, unknown or other
  source 1..775: mol_type = other DNA
nnnnnnnnnnnnnnnnnnnnnncangttctgccttcttctttntcctacagctcctgggcaacgccaccatggacaactttgtatacaaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttaaaaggcacagagagaatggaagatcagggtcagagtattatttccaatgcttactggagaagtgattcctgtaatggaactgctttcatctatgaaatcacacagtgttcctgaagaaatagatatagctgatacagtactcaatgatgatgatattgnngacagctgtcatgaaggctttctttcnncgnaagt

SEQ ID NO: 67 - DNA, 601 bp, synthetic construct
  misc_feature 1..601: note = Description of Artificial Sequence: Synthetic polynucleotide
  misc_difference 1..19: note = modified_base - a, c, t, g, unknown or other
  misc_difference 21..22: note = modified_base - a, c, t, g, unknown or other
  misc_difference 266: note = modified_base - a, c, t, g, unknown or other
  misc_difference 269: note = modified_base - a, c, t, g, unknown or other
  misc_difference 272: note = modified_base - a, c, t, g, unknown or other
  misc_difference 275: note = modified_base - a, c, t, g, unknown or other
  misc_difference 279: note = modified_base - a, c, t, g, unknown or other
  misc_difference 297: note = modified_base - a, c, t, g, unknown or other
  misc_difference 302: note = modified_base - a, c, t, g, unknown or other
  misc_difference 304..305: note = modified_base - a, c, t, g, unknown or other
  misc_difference 318: note = modified_base - a, c, t, g, unknown or other
  misc_difference 327: note = modified_base - a, c, t, g, unknown or other
  misc_difference 349: note = modified_base - a, c, t, g, unknown or other
  misc_difference 357: note = modified_base - a, c, t, g, unknown or other
  misc_difference 361: note = modified_base - a, c, t, g, unknown or other
  misc_difference 367: note = modified_base - a, c, t, g, unknown or other
  misc_difference 379: note = modified_base - a, c, t, g, unknown or other
  misc_difference 381: note = modified_base - a, c, t, g, unknown or other
  misc_difference 383..385: note = modified_base - a, c, t, g, unknown or other
  misc_difference 395: note = modified_base - a, c, t, g, unknown or other
  misc_difference 398..402: note = modified_base - a, c, t, g, unknown or other
  misc_difference 423: note = modified_base - a, c, t, g, unknown or other
  misc_difference 439: note = modified_base - a, c, t, g, unknown or other
  misc_difference 442: note = modified_base - a, c, t, g, unknown or other
  misc_difference 456: note = modified_base - a, c, t, g, unknown or other
  misc_difference 466..467: note = modified_base - a, c, t, g, unknown or other
  misc_difference 469: note = modified_base - a, c, t, g, unknown or other
  misc_difference 473: note = modified_base - a, c, t, g, unknown or other
  misc_difference 475..476: note = modified_base - a, c, t, g, unknown or other
  misc_difference 479..481: note = modified_base - a, c, t, g, unknown or other
  misc_difference 485..488: note = modified_base - a, c, t, g, unknown or other
  misc_difference 501: note = modified_base - a, c, t, g, unknown or other
  misc_difference 508: note = modified_base - a, c, t, g, unknown or other
  misc_difference 533: note = modified_base - a, c, t, g, unknown or other
  misc_difference 569: note = modified_base - a, c, t, g, unknown or other
  misc_difference 572..573: note = modified_base - a, c, t, g, unknown or other
  misc_difference 582: note = modified_base - a, c, t, g, unknown or other
  misc_difference 584: note = modified_base - a, c, t, g, unknown or other
  misc_difference 593..595: note = modified_base - a, c, t, g, unknown or other
  source 1..601: mol_type = other DNA
nnnnnnnnnnnnnnnnnnntnnagcagcgtatccacatagcgtaaaaggagcaacatagttaagaataccagtcaatctttcacaaattttgtaatccagaggttgatttcgataacttcgtataggatactttatacgaagttattgcgattcggataacttcgtatagcatacattatacgaagttatcaaggctacaactttattatacaaagttgtttaggcgtagtcgggcacgtcgtaggggtagtggtggtggtggtgnccnccntgnacanaatctactgtatcaccanaagangnnccatggccatggncgaactcanaatgtctgatggggcagaacancttcatcnacancttccnactgctcaccanantnnnaagcctgtgnacnnnnnaccccaagaccataatactgntgaacgtgcccctgcnccnaccatcctgaccanacccctgctnnanaccnanntannnatcnnnnccctaatcctganatgccangagagaatctctccccaccacctgnacagatgccacagccaggacctaccccaggaaatgnccnntgccaccancntaacctttnnnctacta

SEQ ID NO: 68 - DNA, 8789 bp, synthetic construct
  misc_feature 1..8789: note = Description of Artificial Sequence: Synthetic polynucleotide
  source 1..8789: mol_type = other DNA
agtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggacaactttgtatacaaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgacgagcagaagctgatctccgaggaggacctgtgaccacccaacttttctatacaaagttgtagtatccaaggtagtggactagtgtgacgctgctgacccctttctttcccttctgcagaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactacccctacgacgtgcccgactacgcctaaacaactttgtataataaagttgtagccttgataacttcgtataatgtatgctatacgaagttatccgaatcgcaataacttcgtataaagtatcctatacgaagttatcgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagt

SEQ ID NO: 69 - DNA, 1086 bp, synthetic construct
  misc_feature 1..1086: note = Description of Artificial Sequence: Synthetic polynucleotide
  misc_difference 1..26: note = modified_base - a, c, t, g, unknown or other
  misc_difference 28..29: note = modified_base - a, c, t, g, unknown or other
  misc_difference 32: note = modified_base - a, c, t, g, unknown or other
  misc_difference 845: note = modified_base - a, c, t, g, unknown or other
  misc_difference 907: note = modified_base - a, c, t, g, unknown or other
  misc_difference 929: note = modified_base - a, c, t, g, unknown or other
  misc_difference 991: note = modified_base - a, c, t, g, unknown or other
  misc_difference 1004: note = modified_base - a, c, t, g, unknown or other
  misc_difference 1006: note = modified_base - a, c, t, g, unknown or other
  misc_difference 1018: note = modified_base - a, c, t, g, unknown or other
  misc_difference 1020: note = modified_base - a, c, t, g, unknown or other
  misc_difference 1025..1026: note = modified_base - a, c, t, g, unknown or other
  misc_difference 1047: note = modified_base - a, c, t, g, unknown or other
  misc_difference 1052: note = modified_base - a, c, t, g, unknown or other
  misc_difference 1056: note = modified_base - a, c, t, g, unknown or other
  misc_difference 1058: note = modified_base - a, c, t, g, unknown or other
  misc_difference 1060: note = modified_base - a, c, t, g, unknown or other
  misc_difference 1064: note = modified_base - a, c, t, g, unknown or other
  misc_difference 1066..1067: note = modified_base - a, c, t, g, unknown or other
  misc_difference 1075..1080: note = modified_base - a, c, t, g, unknown or other
  misc_difference 1084..1086: note = modified_base - a, c, t, g, unknown or other
  source 1..1086: mol_type = other DNA
nnnnnnnnnnnnnnnnnnnnnnnnnngnnctnccttcttctttttcctacagctcctgggcaacgccaccatggacaactttgtatacaaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgacgagcagaagctgatctccgangaggacctgtgaccacccaacttttctatacaaagttgtagtatccaaggtagtggactagngtgacgctgctgacccctttcntttcccttctgcagaatgccatcagctcacacttgcaaacctgtggctgttccgttgtagtnggtagcagtgcananaaagtaaataananagtcnnaacattatgcctttttctganttccngcanananaaangnnccaggttnnnnnngaannn

SEQ ID NO: 70 - DNA, 938 bp, synthetic construct
  misc_feature 1..938: note = Description of Artificial Sequence: Synthetic polynucleotide
  misc_difference 1..13: note = modified_base - a, c, t, g, unknown or other
  misc_difference 15: note = modified_base - a, c, t, g, unknown or other
  misc_difference 18..22: note = modified_base - a, c, t, g, unknown or other
  misc_difference 862..863: note = modified_base - a, c, t, g, unknown or other
  misc_difference 866: note = modified_base - a, c, t, g, unknown or other
  misc_difference 869: note = modified_base - a, c, t, g, unknown or other
  misc_difference 892..893: note = modified_base - a, c, t, g, unknown or other
  misc_difference 902: note = modified_base - a, c, t, g, unknown or other
  misc_difference 904..905: note = modified_base - a, c, t, g, unknown or other
  misc_difference 910..911: note = modified_base - a, c, t, g, unknown or other
  misc_difference 915: note = modified_base - a, c, t, g, unknown or other
  misc_difference 917: note = modified_base - a, c, t, g, unknown or other
  misc_difference 925: note = modified_base - a, c, t, g, unknown or other
  misc_difference 927..928: note = modified_base - a, c, t, g, unknown or other
  misc_difference 931: note = modified_base - a, c, t, g, unknown or other
  misc_difference 933: note = modified_base - a, c, t, g, unknown or other
  misc_difference 936: note = modified_base - a, c, t, g, unknown or other
  source 1..938: mol_type = other DNA
nnnnnnnnnnnnngnatnnnnnagcgtatccacatagcgtaaaaggagcaacatagttaagaataccagtcaatctttcacaaattttgtaatccagaggttgatttcgataacttcgtataggatactttatacgaagttattgcgattcggataacttcgtatagcatacattatacgaagttatcaaggctacaactttattatacaaagttgtttaggcgtagtcgggcacgtcgtaggggtagtggtggtggtggtggtgaaaagtcattagaacatctcgttcttgcacactagtgtagaaaggtcttccaaagataaaagagtgtaggcctggtttaattttctcagccagagccattattatgttaagatcgccctctgctgttaaatcaaggtctatcttcaggttccgaagagatttaaagggcttttttcccttctgcgtatcgtcttctatatattttattagtgtcaaggcttttctgtgaaggacaagtagaaactgtgcaaggaaagtacttctgagagataagccaggtttcagctgaaagacctgatccaggaaggctttcactagagtgtctctgtgtaagacatcttgaaaaatattcaaatcaggagtaaagctttcgtcagtgtagatgatcgtatcctgagccatgtcttcttctgaagtggctctccagaaggctgtcagctcggatctcatgtatctacgctgattataaatatgttcatgacagggtggcatctgcttcacagtattgacatccacatctatgtgtgtggtgggatatggagcatacatgacttgccggaaaggcagcacaaagcttccagttgaatccttttagcnngcntgnacaaagagccctgactcatattnnaatgatgantnngcttnncatnancctggaancnnttncnctntg

SEQ ID NO: 71 - DNA, 8738 bp, synthetic construct
  misc_feature 1..8738: note = Description of Artificial Sequence: Synthetic polynucleotide
  source 1..8738: mol_type = other DNA
agtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggcacccaacttttctatacaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgacgagcagaagctgatctccgaggaggacctgtgacgtatccaaggtagtggactagtgtgacgctgctgacccctttctttcccttctgcagaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactaaacaactttgtataataaagttgtagccttgataacttcgtataatgtatgctatacgaagttatccgaatcgcaataacttcgtataaagtatcctatacgaagttatcgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagt

SEQ ID NO: 72 - DNA, 936 bp, synthetic construct
  misc_feature 1..936: note = Description of Artificial Sequence: Synthetic polynucleotide
  misc_difference 1..27: note = modified_base - a, c, t, g, unknown or other
  misc_difference 29..30: note = modified_base - a, c, t, g, unknown or other
  misc_difference 34..35: note = modified_base - a, c, t, g, unknown or other
  misc_difference 859: note = modified_base - a, c, t, g, unknown or other
  misc_difference 878: note = modified_base - a, c, t, g, unknown or other
  source 1..936: mol_type = other DNA
nnnnnnnnnnnnnnnnnnnnnnnnnnnanntgtnntgccttcttctttttcctacagctcctgggcaacgccaccatggcacccaacttttctatacaaagttgtagccaccatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgacgagcagaagctgatctccgaggagganctgtgacgtatccaaaggnagtggactagtgtgacgctgctgacccctttctttcccttctgcagaatgccatcagc

SEQ ID NO: 73 - DNA, 846 bp, synthetic construct
  misc_feature 1..846: note = Description of Artificial Sequence: Synthetic polynucleotide
  misc_difference 1..17: note = modified_base - a, c, t, g, unknown or other
  misc_difference 24: note = modified_base - a, c, t, g, unknown or other
  misc_difference 87: note = modified_base - a, c, t, g, unknown or other
  source 1..846: mol_type = other DNA
nnnnnnnnnnnnnnnnngcattanagcagcgtatccacatagcgtaaaaggagcaacatagttaagaataccagtcaatctttcacnaattttgtaatccagaggttgatttcgataacttcgtataggatactttatacgaagttattgcgattcggataacttcgtatagcatacattatacgaagttatcaaggctacaactttattatacaaagttgtttagtggtggtggtggtggtgaaaagtcattagaacatctcgttcttgcacactagtgtagaaaggtcttccaaagataaaagagtgtaggcctggtttaattttctcagccagagccattattatgttaagatcgccctctgctgttaaatcaaggtctatcttcaggttccgaagagatttaaagggcttttttcccttctgcgtatcgtcttctatatattttattagtgtcaaggcttttctgtgaaggacaagtagaaactgtgcaaggaaagtacttctgagagataagccaggtttcagctgaaagacctgatccaggaaggctttcactagagtgtctctgtgtaagacatcttgaaaaatattcaaatcaggagtaaagctttcgtcagtgtagatgatcgtatcctgagccatgtcttcttctgaagtggctctccagaaggctgtcagctcggatctcatgtatctacgctgattataaatatgttcatgacagggtggcatctgcttcacagtattgacatccacatctatgtgtgtggtgggatatggagcatacatgacttgccggaaaggcagcacaaagcttccagttgaatcctttagcaggccttg

SEQ ID NO: 74 - DNA, 6376 bp, synthetic construct
  misc_feature 1..6376: note = Description of Artificial Sequence: Synthetic polynucleotide
  source 1..6376: mol_type = other DNA
ccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcgcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatgagggagctccaaagaagaagcgtaaggtaggtagttcctagacaactttgtatacaaaagttgtattaaagggaggtagtgagtcgaccagtggatcctggaggcttgctgaaggctgtatgctttcagtgtcagcctttcatacgttttggccactgactgacgtatgaaactgacactgaagacacaaggcctgttactagcactcacatggaacaaatggcccagatctggccgcactcgagatatctagaacccagctttcttgtacaaagtggttgatcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaag

SEQ ID NO: 75 - DNA, 870 bp, synthetic construct
  misc_feature 1..870: note = Description of Artificial Sequence: Synthetic polynucleotide
  misc_difference 1..14: note = modified_base - a, c, t, g, unknown or other
  misc_difference 19..23: note = modified_base - a, c, t, g, unknown or other
  misc_difference 26: note = modified_base - a, c, t, g, unknown or other
  misc_difference 82: note = modified_base - a, c, t, g, unknown or other
  misc_difference 649: note = modified_base - a, c, t, g, unknown or other
  misc_difference 677: note = modified_base - a, c, t, g, unknown or other
  misc_difference 771..773: note = modified_base - a, c, t, g, unknown or other
  misc_difference 780: note = modified_base - a, c, t, g, unknown or other
  misc_difference 787: note = modified_base - a, c, t, g, unknown or other
  misc_difference 793: note = modified_base - a, c, t, g, unknown or other
  misc_difference 796: note = modified_base - a, c, t, g, unknown or other
  misc_difference 802..803: note = modified_base - a, c, t, g, unknown or other
  misc_difference 816: note = modified_base - a, c, t, g, unknown or other
  misc_difference 831: note = modified_base - a, c, t, g, unknown or other
  misc_difference 835..836: note = modified_base - a, c, t, g, unknown or other
  misc_difference 846: note = modified_base - a, c, t, g, unknown or other
  misc_difference 851: note = modified_base - a, c, t, g, unknown or other
  misc_difference 855: note = modified_base - a, c, t, g, unknown or other
  misc_difference 862: note = modified_base - a, c, t, g, unknown or other
  misc_difference 865..866: note = modified_base - a, c, t, g, unknown or other
  misc_difference 870: note = modified_base - a, c, t, g, unknown or other
  source 1..870: mol_type = other DNA
nnnnnnnnnnnnnnatcgnnnnnagntattaatagtaatcaattacggggtcattagttcatagcccatatatggagttccncgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcgaaaaggtgcggcggcagccaatcagagcggcgcgctccgaaagtttccttttatggcgaggcggcggcggcggcggccctataaaaagcnaagcgcgcggcgggcgggagtcgctgcncgctgccttcgccccgtgccccgctccgccgccgcctcgcgccgcccgccccggctctgactgaccgcgttactcccacaggtgagcgggcggnnnggccctnctcctcnggctgnatngcgctnnttaatgacggctngtttcttttctgtgntgcnngaagccttgnggggntccngggaggnccnnttgn

SEQ ID NO: 76 - DNA, 908 bp, synthetic construct
  misc_feature 1..908: note = Description of Artificial Sequence: Synthetic polynucleotide
  misc_difference 1..13: note = modified_base - a, c, t, g, unknown or other
  misc_difference 15: note = modified_base - a, c, t, g, unknown or other
  misc_difference 17: note = modified_base - a, c, t, g, unknown or other
  misc_difference 19: note = modified_base - a, c, t, g, unknown or other
  misc_difference 43: note = modified_base - a, c, t, g, unknown or other
  misc_difference 892: note = modified_base - a, c, t, g, unknown or other
  misc_difference 907..908: note = modified_base - a, c, t, g, unknown or other
  source 1..908: mol_type = other DNA
nnnnnnnnnnnnngngngnggcagatctgggccatttgttccntgtgagtgctagtaacaggccttgtgtcttcagtgtcagtttcatacgtcagtcagtggccaaaacgtatgaaaggctgacactgaaagcatacagccttcagcaagcctccaggatccactggtcgactcactacctccctttaatacaacttttgtatacaaagttgtctaggaactacctaccttacgcttcttctttggagctccctcattaagcttgtgccccagtttgctagggaggtcgcagtatctggccactgccacctcgtgctgctcgacgtaggtctcgttgttggcctccttgattctttccagtctgtagtccacatagtagacgccaggcatcttgaggttcttagcgggtttcttggatctatatgtggtcttgatgtttgcgatcagatggctcccgcccacgagcttcagggccatgtcgtttctgccttccaggccgccgtcagcggggtacagcgtctcggtgaaggcctcccagccgagtgttttcttctgcatcacagggccgttggatgtgaagttcacccctctgatcttgacgttgtagatgaggcagccgtcctggaggctggtgtcctgggtagcggtcagcacgcccccgtcttcgtatgtggtgactctctcccatgtgaagccctcagggaaggactgcttgaagaagtcggggatgccctgggtgtggttgatgaaggtcttgctgccgtagaggaagctagtagccaggatgtcgaaggcgaaggggagagggccgccctcgaccaccttgattctcatggtctgggtgccctcgtagggcttgccttcgccctcggatgtgcacttgaagtgatgnttgtccacggtgccnn

SEQ ID NO: 77 - DNA, 6376 bp, synthetic construct
  misc_feature 1..6376: note = Description of Artificial Sequence: Synthetic polynucleotide
  source 1..6376: mol_type = other DNA
ccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcgcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattctctggctaactagagaacccactgcttactggcttatcgaaattaatacgactcactatagggagacccaagctggctagttaagctatcaacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatgagggagctccaaagaagaagcgtaaggtaggtagttcctagacaactttgtatacaaaagttgtattaaagggaggtagtgagtcgaccagtggatcctggaggcttgctgaaggctgtatgcttagtatgtatgacaaagtcctgttttggccactgactgacaggactttcatacatactagacacaaggcctgttactagcactcacatggaacaaatggcccagatctggccgcactcgagatatctagaacccagctttcttgtacaaagtggttgatcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaag

SEQ ID NO: 78 - DNA, 953 bp, synthetic construct
  misc_feature 1..953: note = Description of Artificial Sequence: Synthetic polynucleotide
  misc_difference 1..14: note = modified_base - a, c, t, g, unknown or other
  misc_difference 16..21: note = modified_base - a, c, t, g, unknown or other
  misc_difference 79: note = modified_base - a, c, t, g, unknown or other
  misc_difference 534: note = modified_base - a, c, t, g, unknown or other
  misc_difference 645..646: note = modified_base - a, c, t, g, unknown or other
  misc_difference 768: note = modified_base - a, c, t, g, unknown or other
  misc_difference 772: note = modified_base - a, c, t, g, unknown or other
  misc_difference 803..804: note = modified_base - a, c, t, g, unknown or other
  misc_difference 822: note = modified_base - a, c, t, g, unknown or other
  misc_difference 829..830: note = modified_base - a, c, t, g, unknown or other
  misc_difference 835..837: note = modified_base - a, c, t, g, unknown or other
  misc_difference 847: note = modified_base - a, c, t, g, unknown or other
  misc_difference 854..856: note = modified_base - a, c, t, g, unknown or other
  misc_difference 860: note = modified_base - a, c, t, g, unknown or other
  misc_difference 862: note = modified_base - a, c, t, g, unknown or other
  misc_difference 867..869: note = modified_base - a, c, t, g, unknown or other
  misc_difference 874: note = modified_base - a, c, t, g, unknown or other
  misc_difference 877: note = modified_base - a, c, t, g, unknown or other
  misc_difference 879: note = modified_base - a, c, t, g, unknown or other
  misc_difference 886..892: note = modified_base - a, c, t, g, unknown or other
  misc_difference 895..896: note = modified_base - a, c, t, g, unknown or other
  misc_difference 898: note = modified_base - a, c, t, g, unknown or other
  misc_difference 900: note = modified_base - a, c, t, g, unknown or other
  misc_difference 902..904: note = modified_base - a, c, t, g, unknown or other
  misc_difference 906: note = modified_base - a, c, t, g, unknown or other
  misc_difference 908: note = modified_base - a, c, t, g, unknown or other
  misc_difference 910: note = modified_base - a, c, t, g, unknown or other
  misc_difference 913..914: note = modified_base - a, c, t, g, unknown or other
  misc_difference 918: note = modified_base - a, c, t, g, unknown or other
  misc_difference 920: note = modified_base - a, c, t, g, unknown or other
  misc_difference 924..926: note = modified_base - a, c, t, g, unknown or other
  misc_difference 934: note = modified_base - a, c, t, g, unknown or other
  misc_difference 938..940: note = modified_base - a, c, t, g, unknown or other
  misc_difference 943: note = modified_base - a, c, t, g, unknown or other
  misc_difference 947..950: note = modified_base - a, c, t, g, unknown or other
  misc_difference 952..953: note = modified_base - a, c, t, g, unknown or other
  source 1..953: mol_type = other DNA
nnnnnnnnnnnnnngnnnnnngttattaatagtaatcaattacggggtcattagttcatagcccatatatggagttccncgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcnaggggcggggcggggcgaggcgaaaaggtgcggcggcagccaatcagagcggcgcgctccgaaagtttccttttatggcgaggcggcggcggcggcggccctataaaaagnnaagcgcgcggcgggcgggagtcgctgcgcgctgccttcgccccgtgccccgctccgccgccgcctcgcgccgcccgccccggctctgactgaccgcgttactcccacaggtgagcgggcggnacgncccttctcctccgggctgtaattagcgcttnnttaatgacggcttgttcntttctgnngctgnnnaaagccttgnggggctnnnaggncntttgnnnggggnagngntcggggnnnnnnntgnntntntnnngnancnccnngtgngntccnnnctgcccgngctnnnacnctgnnnncnn

SEQ ID NO: 79 - DNA, 958 bp, synthetic construct
  misc_feature 1..958: note = Description of Artificial Sequence: Synthetic polynucleotide
  misc_difference 2..16: note = modified_base - a, c, t, g, unknown or other
  misc_difference 18..19: note = modified_base - a, c, t, g, unknown or other
  misc_difference 80: note = modified_base - a, c, t, g, unknown or other
  misc_difference 935: note = modified_base - a, c, t, g, unknown or other
  misc_difference 958: note = modified_base - a, c, t, g, unknown or other
  source 1..958: mol_type = other DNA
cnnnnnnnnnnnnnnngnngcagatctgggccatttgttccatgtgagtgctagtaacaggccttgtgtctagtatgtangaaagtcctgtcagtcagtggccaaaacaggactttgtcatacatactaagcatacagccttcagcaagcctccaggatccactggtcgactcactacctccctttaatacaacttttgtatacaaagttgtctaggaactacctaccttacgcttcttctttggagctccctcattaagcttgtgccccagtttgctagggaggtcgcagtatctggccactgccacctcgtgctgctcgacgtaggtctcgttgttggcctccttgattctttccagtctgtagtccacatagtagacgccaggcatcttgaggttcttagcgggtttcttggatctatatgtggtcttgatgtttgcgatcagatggctcccgcccacgagcttcagggccatgtcgtttctgccttccaggccgccgtcagcggggtacagcgtctcggtgaaggcctcccagccgagtgttttcttctgcatcacagggccgttggatgtgaagttcacccctctgatcttgacgttgtagatgaggcagccgtcctggaggctggtgtcctgggtagcggtcagcacgcccccgtcttcgtatgtggtgactctctcccatgtgaagccctcagggaaggactgcttgaagaagtcggggatgccctgggtgtggttgatgaaggtcttgctgccgtagaggaagctagtagccaggatgtcgaaggcgaaggggagagggccgccctcgaccaccttgattctcatggtctgggtgccctcgtagggcttgccttcgccctcggatgtgcacttgaagtgatggttgtccacggtgccctccatgtacagcttcatgtgcatgttctnccttaatcagctcgctcatccan

SEQ ID NO: 80 - DNA, 11302 bp, synthetic construct
  misc_feature 1..11302: note = Description of Artificial Sequence: Synthetic polynucleotide
  source 1..11302: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaatggccgcacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggcacccaacttttctatacaaagttgtatccttactctaggaccaagaatgaactgctttcatctatgaaagaagaaatagatgtaagtttaaatgagagcaattatacactttaatgtatattattaatattctaaacatactattcacatacagtaataggagcaattaatatttaatgtagtgtcttttgaaacaaaagagtgttaagagatacctttagaagaggaagttgttcttgtaaaaaaaagtgttatttcaacactatgatacagtactcaatgatgatgataaagtaagaatttttcttttcataaaatagggacattacgtatttgaacactcattatatttctatatataacagaatcctttcatattaagttgtactgtagatgaacttaagttatttaagcagtggagtttagtacttaatataagcattgagtaagataaataatataaaagctaacatttcctatttacatttcttctagacacagttacagattttcatgaaattttagcatgagtgtgtttaacctaaagcctttcatacatcattttaaacatgtcaatttcttcagctacattaattaaatgatattatattatcttcaggttccgaagagaacaactttgtataataaagttgtaatgcatcaccaccatcatcacgattataaggatgacgatgacaagggagctggggcgggtgcgggggcaggagccggagccggcgcgggcgcaggtgcaggtgctggtgctggcgccggtgcgggagccggggcaggcgctggggcgggcgctggtgctggtgctggtgccggggccggcgccggagcaggggctggagcgggcgcgggggcgggcgccggagccggtgcgggggccggggccggcgcaggcgcaggcgctggcgccggtgctggagctggcgccggggcgggagcaggggccggagcaggcgctggtgccggcgcaggggctggcgcgggggcaggtgcaggcgcaggtgccggtgccggggcaggcgctggcgctggtgccggcgcaggggcaggggcaggagcgggcgcaggtgcgggggctggtgccggtgctggagctggggcaggggcgggcgcaggtgccggcgcgggtgccggtgccggcgccggggccggggccggggcaggcgctcatcaccaccatcatcacgattataaggatgacgatgacaagagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaacccagctttcttgtacaaagtggtgcggccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 81 - DNA, 1077 bp, synthetic construct
  misc_feature 1..1077: note = Description of Artificial Sequence: Synthetic polynucleotide
  misc_difference 1..20: note = modified_base - a, c, t, g, unknown or other
  misc_difference 22..24: note = modified_base - a, c, t, g, unknown or other
  misc_difference 26: note = modified_base - a, c, t, g, unknown or other
  misc_difference 821..823: note = modified_base - a, c, t, g, unknown or other
  misc_difference 826: note = modified_base - a, c, t, g, unknown or other
  misc_difference 828: note = modified_base - a, c, t, g, unknown or other
  misc_difference 853: note = modified_base - a, c, t, g, unknown or other
  misc_difference 861..862: note = modified_base - a, c, t, g, unknown or other
  misc_difference 903: note = modified_base - a, c, t, g, unknown or other
  misc_difference 913: note = modified_base - a, c, t, g, unknown or other
  misc_difference 915: note = modified_base - a, c, t, g, unknown or other
  misc_difference 947: note = modified_base - a, c, t, g, unknown or other
  misc_difference 968: note = modified_base - a, c, t, g, unknown or other
  misc_difference 975..978: note = modified_base - a, c, t, g, unknown or other
  misc_difference 990: note = modified_base - a, c, t, g, unknown or other
  misc_difference 992..994: note = modified_base - a, c, t, g, unknown or other
  misc_difference 999: note = modified_base - a, c, t, g, unknown or other
  misc_difference 1001: note = modified_base - a, c, t, g, unknown or other
  misc_difference 1019: note = modified_base - a, c, t, g, unknown or other
  misc_difference 1025: note = modified_base - a, c, t, g, unknown or other
  misc_difference 1027: note = modified_base - a, c, t, g, unknown or other
  misc_difference 1030: note = modified_base - a, c, t, g, unknown or other
  misc_difference 1045..1050: note = modified_base - a, c, t, g, unknown or other
  misc_difference 1059: note = modified_base - a, c, t, g, unknown or other
  misc_difference 1068: note = modified_base - a, c, t, g, unknown or other
  misc_difference 1070: note = modified_base - a, c, t, g, unknown or other
  misc_difference 1075..1077: note = modified_base - a, c, t, g, unknown or other
  source 1..1077: mol_type = other DNA
nnnnnnnnnnnnnnnnnnnnannngntctgccttcttctttttcctacagctcctgggcaacgccaccatggcacccaacttttctatacaaagttgtatccttactctaggaccaagaatgaactgctttcatctatgaaagaagaaatagatgtaagtttaaatgagagcaattatacactttaatgtatattattaatattctaaacatactattcacatacagtaataggagcaattaatatttaatgtagtgtcttttgaaacaaaagagtgttaagagatacctttagaagaggaagttgttcttgtaaaaaaaagtgttatttcaacactatgatacagtactcaatgatgatgataaagtaagaatttttcttttcataaaatagggacattacgtatttgaacactcattatatttctatatataacagaatcctttcatattaagttgtactgtagatgaacttaagttatttaagcagtggagtttagtacttaatataagcattgagtaagataaataatataaaagctaacatttcctatttacatttcttctagacacagttacagattttcatgaaattttagcatgagtgtgtttaacctaaagcctttcatacatcattttaaacatgtcaatttcttcagctacattaattaaatgatattatattatcttcaggttccgaagagaacaactttgtataataaagttgtaatgcatcaccaccatcatcacgattataaggatgacgatgacaagggagctggggcgggtgcgggggcaggagccggagccggcgcgggcgcnnngcngngctggtgctggcgccggtgcggganccggggcnncgctggggcgggcgctggtgctggtgctggtgccggggccngcgcccggancnagggctggagcgggcgcgggggcgggcgccgnagccggtgcgggggccggggncggcgcnnnncagcgctggccncnnngctgnanctggcgccggggcgggancagggncnganaggcgctggtgccgnnnnnngggctggcncggggcagntncaggnnn

SEQ ID NO: 82 - DNA, 1045 bp, synthetic construct
  misc_feature 1..1045: note = Description of Artificial Sequence: Synthetic polynucleotide
  misc_difference 1..13: note = modified_base - a, c, t, g, unknown or other
  misc_difference 15..22: note = modified_base - a, c, t, g, unknown or other
  misc_difference 33: note = modified_base - a, c, t, g, unknown or other
  misc_difference 886: note = modified_base - a, c, t, g, unknown or other
  misc_difference 902: note = modified_base - a, c, t, g, unknown or other
  misc_difference 911: note = modified_base - a, c, t, g, unknown or other
  misc_difference 924: note = modified_base - a, c, t, g, unknown or other
  misc_difference 933: note = modified_base - a, c, t, g, unknown or other
  misc_difference 935: note = modified_base - a, c, t, g, unknown or other
  misc_difference 967: note = modified_base - a, c, t, g, unknown or other
  misc_difference 976..977: note = modified_base - a, c, t, g, unknown or other
  misc_difference 980: note = modified_base - a, c, t, g, unknown or other
  misc_difference 983: note = modified_base - a, c, t, g, unknown or other
  misc_difference 985: note = modified_base - a, c, t, g, unknown or other
  misc_difference 992..993: note = modified_base - a, c, t, g, unknown or other
  misc_difference 996: note = modified_base - a, c, t, g, unknown or other
  misc_difference 998..999: note = modified_base - a, c, t, g, unknown or other
  misc_difference 1001: note = modified_base - a, c, t, g, unknown or other
  misc_difference 1005..1012: note = modified_base - a, c, t, g, unknown or other
  misc_difference 1015..1016: note = modified_base - a, c, t, g, unknown or other
  misc_difference 1018: note = modified_base - a, c, t, g, unknown or other
  misc_difference 1020: note = modified_base - a, c, t, g, unknown or other
  misc_difference 1023: note = modified_base - a, c, t, g, unknown or other
  misc_difference 1028..1029: note = modified_base - a, c, t, g, unknown or other
  misc_difference 1032..1033: note = modified_base - a, c, t, g, unknown or other
  misc_difference 1036: note = modified_base - a, c, t, g, unknown or other
  misc_difference 1038..1039: note = modified_base - a, c, t, g, unknown or other
  misc_difference 1041..1043: note = modified_base - a, c, t, g, unknown or other
  misc_difference 1045: note = modified_base - a, c, t, g, unknown or other
  source 1..1045: mol_type = other DNA
nnnnnnnnnnnnngnnnnnnnncagcgtatccncatagcgtaaaaggagcaacatagttaagaataccagtcaatctttcacaaattttgtaatccagaggttgatttcacttgtacagctcgtccatgccatgtgtgatcccagcagcggtcacaaactccagcaggaccatgtggtctctcttttcgttgggatctttagacagggcagactgggtggacaggtaatggttgtctgggaggagcacagggccgtcgccgattggagtgttctgttgataatggtcggccagctgcacggatccatcctcaatgttgtgtctgatcttgaagttgaccttgatgccattcttttgcttgtcggccatgatgtacacattgtgggagttatagttgtattccagcttgtggccgagaatgtttccatcctccttaaagtcaatgcccttcagctcgattctattcaccagggtgtcaccttcgaacttgacttcagcgcgggtcttgtagttcccgtcatctttgaaaaagatggttctctcctgcacatagccctcgggcatggcgctcttgaaaaagtcatgctgcttcatatggtctgggtatctggaaaagcactgcacgccataggtcagggtagtgaccagtgttggccatggcacagggagctttccagtggtgcagatgaatttcagggtgagctttccgtatgtggcatcaccttcaccctctccgctgacagaaaatttgtgcccattcacatcgccatccagttccacgagaattgggaccacgccagtgaacagttcctcgcccttgctcttgtcatcgtcatccttataatcgtgatgatggtggtgatgagcgcctgccccggccccggccncggcgccggcaccggnacccgcgcngcacctgcgcccnccctgcccnanctcagcaccggcaccagccccgcactgcgccnctctgcccnnccngcncngcaccanngcngnncngccnnnnnnnntgnncngnacngcccnngcnnccngnncnnnan

SEQ ID NO: 83 - DNA, 11285 bp, synthetic construct
  misc_feature 1..11285: note = Description of Artificial Sequence: Synthetic polynucleotide
  source 1..11285: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaatggccgcacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggcacccaacttttctatacaaagttgtatccttactctaggaccaagaatccatacatgcagacatgattacattaattaacatgaggttttgctttttctttaatccctgattggtatttagaaaccactgctattgtagtgaaaattctacaatcataaagccctcacttcttgttttttacccggctaagtttttaatttttcctggctctcaatacttgtaagacagtgaactgtttacagtaccagaaagttcacaacactttctcaatcttcaatggaaggtgaagttcatatcactatcctgggaactatctaattaacgtagaatagaatgccaacatagccaaacaaaatattttatcaactcgttcttgtttcagatgtatagcagtttccaactgattcaaccgtatttcaagtattctgagatagtcttgtttctgtgatattcacagattatgttaaaagtttctctgagaaaaatcatatcttaatgcatggcaactgtttgaatagaaatttaccccctcctgtttctgaatacaaatctgtgcacttctttagacaatccttgttttcttctggttaattatcttcaggttccgaagagaacaactttgtataataaagttgtaatgcatcaccaccatcatcacgattataaggatgacgatgacaagggagctggggcgggtgcgggggcaggagccggagccggcgcgggcgcaggtgcaggtgctggtgctggcgccggtgcgggagccggggcaggcgctggggcgggcgctggtgctggtgctggtgccggggccggcgccggagcaggggctggagcgggcgcgggggcgggcgccggagccggtgcgggggccggggccggcgcaggcgcaggcgctggcgccggtgctggagctggcgccggggcgggagcaggggccggagcaggcgctggtgccggcgcaggggctggcgcgggggcaggtgcaggcgcaggtgccggtgccggggcaggcgctggcgctggtgccggcgcaggggcaggggcaggagcgggcgcaggtgcgggggctggtgccggtgctggagctggggcaggggcgggcgcaggtgccggcgcgggtgccggtgccggcgccggggccggggccggggcaggcgctcatcaccaccatcatcacgattataaggatgacgatgacaagagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaacccagctttcttgtacaaagtggtgcggccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 84 - DNA, 1028 bp, synthetic construct
  misc_feature 1..1028: note = Description of Artificial Sequence: Synthetic polynucleotide
  misc_difference 1..12: note = modified_base - a, c, t, g, unknown or other
  misc_difference 14: note = modified_base - a, c, t, g, unknown or other
  misc_difference 16: note = modified_base - a, c, t, g, unknown or other
  misc_difference 19..22: note = modified_base - a, c, t, g, unknown or other
  misc_difference 27..28: note = modified_base - a, c, t, g, unknown or other
  misc_difference 774: note = modified_base - a, c, t, g, unknown or other
  misc_difference 782: note = modified_base - a, c, t, g, unknown or other
  misc_difference 791: note = modified_base - a, c, t, g, unknown or other
  misc_difference 806: note = modified_base - a, c, t, g, unknown or other
  misc_difference 847: note = modified_base - a, c, t, g, unknown or other
  misc_difference 898: note = modified_base - a, c, t, g, unknown or other
  misc_difference 932: note = modified_base - a, c, t, g, unknown or other
  misc_difference 958..959: note = modified_base - a, c, t, g, unknown or other
  misc_difference 963: note = modified_base - a, c, t, g, unknown or other
  misc_difference 984: note = modified_base - a, c, t, g, unknown or other
  misc_difference 990: note = modified_base - a, c, t, g, unknown or other
  misc_difference 997: note = modified_base - a, c, t, g, unknown or other
  misc_difference 1003: note = modified_base - a, c, t, g, unknown or other
  misc_difference 1009..1011: note = modified_base - a, c, t, g, unknown or other
  misc_difference 1014: note = modified_base - a, c, t, g, unknown or other
  misc_difference 1016: note = modified_base - a, c, t, g, unknown or other
  misc_difference 1018: note = modified_base - a, c, t, g, unknown or other
  misc_difference 1022: note = modified_base - a, c, t, g, unknown or other
  misc_difference 1027..1028: note = modified_base - a, c, t, g, unknown or other
  source 1..1028: mol_type = other DNA
nnnnnnnnnnnncncngcnnnntgttnntgccttcttctttttcctacagctcctgggcaacgccaccatggcacccaacttttctatacaaagttgtatccttactctaggaccaagaatccatacatgcagacatgattacattaattaacatgaggttttgctttttctttaatccctgattggtatttagaaaccactgctattgtagtgaaaattctacaatcataaagccctcacttcttgttttttacccggctaagtttttaatttttcctggctctcaatacttgtaagacagtgaactgtttacagtaccagaaagttcacaacactttctcaatcttcaatggaaggtgaagttcatatcactatcctgggaactatctaattaacgtagaatagaatgccaacatagccaaacaaaatattttatcaactcgttcttgtttcagatgtatagcagtttccaactgattcaaccgtatttcaagtattctgagatagtcttgtttctgtgatattcacagattatgttaaaagtttctctgagaaaaatcatatcttaatgcatggcaactgtttgaatagaaatttaccccctcctgtttctgaatacaaatctgtgcacttctttagacaatccttgttttcttctggttaattatcttcaggttccgaagagaacaactttgtataataaagttgtaatgcatcaccaccatcatcacgattataaggatgacgatgacaagggagctggggcgggtgcngggggcangagccgganccggcgcgggcgcangtgcaggtgctggtgctggcgccggtgcgggagccggggcngcgctggggcgggcgctggtgctggtgctggtgccggggccggcgccggancagggctggagcgggcgcggggcgggcgccgganccggtgcgggggccggggccggcgcnncgcngcgctggcgccggtgctgganctggcncccgggncgggancagggnnnggnancnggcnctggnn

SEQ ID NO: 85 - DNA, 1033 bp, synthetic construct
  misc_feature 1..1033: note = Description of Artificial Sequence: Synthetic polynucleotide
  misc_difference 1..14: note = modified_base - a, c, t, g, unknown or other
  misc_difference 16..19: note = modified_base - a, c, t, g, unknown or other
  misc_difference 22..24: note = modified_base - a, c, t, g, unknown or other
  misc_difference 744..745: note = modified_base - a, c, t, g, unknown or other
  misc_difference 781: note = modified_base - a, c, t, g, unknown or other
  misc_difference 919: note = modified_base - a, c, t, g, unknown or other
  misc_difference 921: note = modified_base - a, c, t, g, unknown or other
  misc_difference 935: note = modified_base - a, c, t, g, unknown or other
  misc_difference 951: note = modified_base - a, c, t, g, unknown or other
  misc_difference 953: note = modified_base - a, c, t, g, unknown or other
  misc_difference 960: note = modified_base - a, c, t, g, unknown or other
  misc_difference 966..968: note = modified_base - a, c, t, g, unknown or other
  misc_difference 970: note = modified_base - a, c, t, g, unknown or other
  misc_difference 973: note = modified_base - a, c, t, g, unknown or other
  misc_difference 978: note = modified_base - a, c, t, g, unknown or other
  misc_difference 981: note = modified_base - a, c, t, g, unknown or other
  misc_difference 990..991: note = modified_base - a, c, t, g, unknown or other
  misc_difference 1006: note = modified_base - a, c, t, g, unknown or other
  misc_difference 1014: note = modified_base - a, c, t, g, unknown or other
  misc_difference 1017: note = modified_base - a, c, t, g, unknown or other
  misc_difference 1019: note = modified_base - a, c, t, g, unknown or other
  misc_difference 1026: note = modified_base - a, c, t, g, unknown or other
  misc_difference 1030..1033: note = modified_base - a, c, t, g, unknown or other
  source 1..1033: mol_type = other DNA
nnnnnnnnnnnnnngnnnntannncagcgtatccacatagcgtaaaaggagcaacatagttaagaataccagtcaatctttcacaaattttgtaatccagaggttgatttcacttgtacagctcgtccatgccatgtgtgatcccagcagcggtcacaaactccagcaggaccatgtggtctctcttttcgttgggatctttagacagggcagactgggtggacaggtaatggttgtctgggaggagcacagggccgtcgccgattggagtgttctgttgataatggtcggccagctgcacggatccatcctcaatgttgtgtctgatcttgaagttgaccttgatgccattcttttgcttgtcggccatgatgtacacattgtgggagttatagttgtattccagcttgtggccgagaatgtttccatcctccttaaagtcaatgcccttcagctcgattctattcaccagggtgtcaccttcgaacttgacttcagcgcgggtcttgtagttcccgtcatctttgaaaaagatggttctctcctgcacatagccctcgggcatggcgctcttgaaaaagtcatgctgcttcatatggtctgggtatctggaaaagcactgcacgccataggtcagggtagtgaccagtgttggccatggcacagggagctttccagtggtgcagatgaatttcagggtgagctttccgtatgtggcatcaccttcaccctctccgctgacannaaaatttgtgcccattcacatcgccatccagttccncgagaattgggaccacgccagtgaacagttcctcgcccttgctcttgtcatcgtcatccttataatcgtgatgatggtggtgatgagcgcctgccccggccccggccccggcgccggcaccggcaccccgcgccgggnanctgcgcccgccccngccccaacttcagcancngcaccanccccgnnncntgnccccnctncctgccccnngcccctgcgccgagnaccaacgncangngctctgncccnnnn

SEQ ID NO: 86 - DNA, 11621 bp, synthetic construct
  misc_feature 1..11621: note = Description of Artificial Sequence: Synthetic polynucleotide
  source 1..11621: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaatggccgcacaagtttgtacaaaaaagcaggcttactcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgccaccatggcacccaacttttctatacaaagttgtatctctgtctcgacaagcccagtttctattggtctccttaaacctgtcttgtaaccttgatacttaccaggtggtggcccaggaagccccaggtgtttttgcttatcagatccaggatcagatggccgatgccgctggtgtatggggtgatcaggccgaggccctcgtgtccggcaatgaacatcacggggaacatcagccagctgcagaaaaagacgtaggccatgattttacagatctttctgcacacgcccttaggcagtgtgtggtagctttcgatgtacaccttggcgatctgaaagaagcatgtgacgccgtaaaagagtccgatcatgaagaacagaattttcagagggcccttggtaaaagcggcggtgattccccacacgatgttgccgatgtctgtcacgaggattgtcatggttctcttgctgtactcctcgtgcagtccagtcaggttgctcaggtggatcaggataacggggcaggtcagcagccacatggagtaccgcagccagatcacggcgccgccgttggtctgatacacggtggcagggctgtccacttcgtgaaacagctcgataaagcacttcaccagctcaatcacacacacgtacacttcctcccagccggttgtggccttgaatgagtgccagccgtagaagatcagctgcacgatggccacaatcactgtgaaccactgcaggcccacggcgatcttgtgctgcagctcggtgccgtggttaatgtgaggaaaacaaccatgatcggcgccggctgttgtggcattagatgtctcgccgtgggcgtcggcagcaggggtcaccacggcggcggcagacagcaggcccctgattgtggcctcagcagatggcacagcgcttatgaaggcgtgggtcatggtggcggctgtttccatggtggcacaactttgtataataaagttgtaatgcatcaccaccatcatcacgattataaggatgacgatgacaagggagctggggcgggtgcgggggcaggagccggagccggcgcgggcgcaggtgcaggtgctggtgctggcgccggtgcgggagccggggcaggcgctggggcgggcgctggtgctggtgctggtgccggggccggcgccggagcaggggctggagcgggcgcgggggcgggcgccggagccggtgcgggggccggggccggcgcaggcgcaggcgctggcgccggtgctggagctggcgccggggcgggagcaggggccggagcaggcgctggtgccggcgcaggggctggcgcgggggcaggtgcaggcgcaggtgccggtgccggggcaggcgctggcgctggtgccggcgcaggggcaggggcaggagcgggcgcaggtgcgggggctggtgccggtgctggagctggggcaggggcgggcgcaggtgccggcgcgggtgccggtgccggcgccggggccggggccggggcaggcgctcatcaccaccatcatcacgattataaggatgacgatgacaagagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaacccagctttcttgtacaaagtggtgcggccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 87 - DNA, 801 bp, synthetic construct
  misc_feature 1..801: note = Description of Artificial Sequence: Synthetic polynucleotide
  misc_difference 1..25: note = modified_base - a, c, t, g, unknown or other
  misc_difference 744: note = modified_base - a, c, t, g, unknown or other
  misc_difference 755: note = modified_base - a, c, t, g, unknown or other
  misc_difference 761..763: note = modified_base - a, c, t, g, unknown or other
  misc_difference 775: note = modified_base - a, c, t, g, unknown or other
  misc_difference 780: note = modified_base - a, c, t, g, unknown or other
  misc_difference 784: note = modified_base - a, c, t, g, unknown or other
  misc_difference 788: note = modified_base - a, c, t, g, unknown or other
  misc_difference 790: note = modified_base - a, c, t, g, unknown or other
  misc_difference 798..799: note = modified_base - a, c, t, g, unknown or other
  source 1..801: mol_type = other DNA
nnnnnnnnnnnnnnnnnnnnnnnnngttctgccttcttctttttcctacagctcctgggcaacgccaccatggcacccaacttttctatacaaagttgtatctctgtctcgacaagcccagtttctattggtctccttaaacctgtcttgtaaccttgatacttaccaggtggtggcccaggaagccccaggtgtttttgcttatcagatccaggatcagatggccgatgccgctggtgtatggggtgatcaggccgaggccctcgtgtccggcaatgaacatcacggggaacatcagccagctgcagaaaaagacgtaggccatgattttacagatctttctgcacacgcccttaggcagtgtgtggtagctttcgatgtacaccttggcgatctgaaagaagcatgtgacgccgtaaaagagtccgatcatgaagaacagaattttcagagggcccttggtaaaagcggcggtgattccccacacgatgttgccgatgtctgtcacgaggattgtcatggttctcttgctgtactcctcgtgcagtccagtcaggttgctcaggtggatcaggataacggggcaggtcagcagccacatggagtaccgcagccagatcacggcgccgccgttggtctgatacacggtggcagggctgtccacttcgtgaaacagctcgataaagcacttcaccagctcaatcacacacacgtacacttcctcccagccggttgtggccttgnatgagtgccanccgtannnatcagctgcacnatggncacnatcncngtgaaccnntg

SEQ ID NO: 88 - DNA, 862 bp, synthetic construct
  misc_feature 1..862: note = Description of Artificial Sequence: Synthetic polynucleotide
  misc_difference 1..13: note = modified_base - a, c, t, g, unknown or other
  misc_difference 15..18: note = modified_base - a, c, t, g, unknown or other
  misc_difference 20: note = modified_base - a, c, t, g, unknown or other
  misc_difference 742: note = modified_base - a, c, t, g, unknown or other
  misc_difference 778: note = modified_base - a, c, t, g, unknown or other
  misc_difference 794: note = modified_base - a, c, t, g, unknown or other
  misc_difference 812: note = modified_base - a, c, t, g, unknown or other
  misc_difference 826: note = modified_base - a, c, t, g, unknown or other
  misc_difference 836: note = modified_base - a, c, t, g, unknown or other
  misc_difference 850: note = modified_base - a, c, t, g, unknown or other
  misc_difference 853: note = modified_base - a, c, t, g, unknown or other
  misc_difference 856: note = modified_base - a, c, t, g, unknown or other
  misc_difference 862: note = modified_base - a, c, t, g, unknown or other
  source 1..862: mol_type = other DNA
nnnnnnnnnnnnngnnnnanagcagcgtatccacatagcgtaaaaggagcaacatagttaagaataccagtcaatctttcacaaattttgtaatccagaggttgatttcacttgtacagctcgtccatgccatgtgtgatcccagcagcggtcacaaactccagcaggaccatgtggtctctcttttcgttgggatctttagacagggcagactgggtggacaggtaatggttgtctgggaggagcacagggccgtcgccgattggagtgttctgttgataatggtcggccagctgcacggatccatcctcaatgttgtgtctgatcttgaagttgaccttgatgccattcttttgcttgtcggccatgatgtacacattgtgggagttatagttgtattccagcttgtggccgagaatgtttccatcctccttaaagtcaatgcccttcagctcgattctattcaccagggtgtcaccttcgaacttgacttcagcgcgggtcttgtagttcccgtcatctttgaaaaagatggttctctcctgcacatagccctcgggcatggcgctcttgaaaaagtcatgctgcttcatatggtctgggtatctggaaaagcactgcacgccataggtcagggtagtgaccagtgttggccatggcacagggagctttccagtggtgcagatgaatttcagggtgagctttccgtatgtggcatcaccttcaccctctccgctgacanaaaatttgtgcccattcacatcgccatccagttccncgagaattgggacacnccagtgaacagttcctcnccttgctcttgtcntcgtcattcntataatcggaaganggnggngatgan

SEQ ID NO: 89 - DNA, 1446 bp, Homo sapiens
  source 1..1446: mol_type = unassigned DNA
atgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaa

SEQ ID NO: 90 - DNA, 1446 bp, Homo sapiens; identical to SEQ ID NO: 34
  source 1..1446: mol_type = unassigned DNA

SEQ ID NO: 91 - DNA, 1446 bp, Homo sapiens
  source 1..1446: mol_type = unassigned DNA
atgagcacactgtgccctccacctagccctgccgtggccaagaccgagatcgccctttccggcaagagccccctgctggccgccacattcgcctactgggacaacatcctgggacctagagtgcggcacatttgggcccctaagaccgagcaggtcctgctgagtgatggcgaaatcaccttcctggccaaccacaccctgaacggcgagatcctgcggaacgccgagagcggtgctatcgatgtgaagttcttcgtgctgagcgagaagggcgtgatcatcgtgtccctcatcttcgacggcaactggaacggcgacagatctacatacggcctgtctatcatcctgcctcagaccgaactgtccttctacctgcctctgcaccgggtgtgcgtggaccggctgactcacatcatcagaaagggcagaatctggatgcacaaggaacggcaggagaacgtgcaaaagatcattctggaaggtacagagagaatggaagatcagggccagagcattatccctatgctgacaggcgaggtgatccccgtgatggaactgttgtcctccatgaagtcccactctgttcctgaggaaatcgacatcgccgacacagtgctgaacgacgacgatatcggcgacagctgccacgagggcttcctgctgaacgccatcagcagccacctgcagacctgtggctgtagcgtggtcgtgggctctagcgccgaaaaggtgaacaagatcgtgcggaccctgtgtctgttcctgacacctgctgagagaaagtgcagcagactgtgcgaggccgagtctagctttaagtacgagagcggcctgttcgtgcagggcctcctgaaggacagcaccggcagctttgtgctgcccttcagacaggtgatgtacgccccttaccccaccacccacatcgacgtggacgtgaacaccgtgaagcagatgcctccgtgccatgagcacatctacaaccagagaagatacatgagaagcgagctgaccgctttctggcgggccacctctgaggaagatatggcccaggacaccatcatctatacagacgagagcttcacccctgatctgaatattttccaagatgtgctgcacagagatacacttgtgaaagccttcctcgaccaggtgttccagctgaagcctggcctgtctctgcggagcacctttctggcacagttcctgctggtgctgcatagaaaggccctgaccttgatcaagtacatcgaggatgacacccagaaaggaaagaaacctttcaagagcctgagaaacctgaaaatcgacctggacctgacggccgaaggcgatctgaatatcatcatggccctggccgagaagatcaagcccggcctgcacagcttcatctttggaagacctttttacaccagcgtgcaggagcgggacgtgctgatgacattttga

SEQ ID NO: 92 - DNA, 1446 bp, Homo sapiens; identical to SEQ ID NO: 35
  source 1..1446: mol_type = unassigned DNA

SEQ ID NO: 93 - DNA, 1446 bp, Homo sapiens; identical to SEQ ID NO: 36
  source 1..1446: mol_type = unassigned DNA

SEQ ID NO: 94 - DNA, 1446 bp, Homo sapiens; identical to SEQ ID NO: 37
  source 1..1446: mol_type = unassigned DNA

SEQ ID NO: 95 - DNA, 1446 bp, Homo sapiens; identical to SEQ ID NO: 38
  source 1..1446: mol_type = unassigned DNA

SEQ ID NO: 96 - DNA, 1446 bp, Homo sapiens; identical to SEQ ID NO: 39
  source 1..1446: mol_type = unassigned DNA

SEQ ID NO: 97 - DNA, 1446 bp, Homo sapiens; identical to SEQ ID NO: 40
  source 1..1446: mol_type = unassigned DNA

SEQ ID NO: 98 - DNA, 1446 bp, Homo sapiens; identical to SEQ ID NO: 41
  source 1..1446: mol_type = unassigned DNA

SEQ ID NO: 99 - DNA, 1446 bp, Homo sapiens; identical to SEQ ID NO: 42
  source 1..1446: mol_type = unassigned DNA

SEQ ID NO: 100 - DNA, 1446 bp, synthetic construct
  misc_feature 1..1446: note = Description of Artificial Sequence: Synthetic polynucleotide
  source 1..1446: mol_type = other DNA
atgagcaccctgtgtcctccacctagccccgccgtggccaagacagagatcgccctgagcggaaaaagccctctgctggccgctacatttgcctactgggacaacatcctgggccctagagtgcggcacatttgggcccctaagaccgaacaggtgctgctgagtgatggagagatcaccttcctggctaatcacacccttaacggcgaaatcctgcggaacgccgagagcggagccatcgacgtgaagttcttcgtgttaagcgagaagggcgtgatcattgtgtccctgatcttcgacggcaactggaacggcgatagatctacatacggcctgtccatcattcttccacagacagagctgtctttctacctgcctctgcaccgggtgtgcgtggacagactgacccacattattagaaaaggcagaatctggatgcacaaggaacggcaggagaacgtgcaaaagatcatcctcgagggtacagagagaatggaagatcagggccagagcatcatccccatgctgaccggcgaggtgatccctgtgatggaactgctgagcagcatgaaaagccactctgtccccgaggaaatcgacatcgccgacaccgtgctgaacgacgatgatataggagattcatgccacgagggcttcctgctgaatgccatcagctctcacctgcagacctgtggctgcagcgtcgtggtgggcagcagcgccgagaaagtgaacaagatcgtgcggaccctgtgcctgttcctgacccctgctgaaagaaagtgcagcagactgtgtgaagccgaatctagctttaagtacgagtctggactgtttgtgcagggcctgctgaaggacagcacaggctccttcgtgctgcccttcagacaggttatgtacgccccttaccccaccacccacatcgatgtggacgtcaacacagtgaagcagatgcctccttgccacgagcacatctacaaccagcgtagatacatgcggagcgagctgaccgccttttggcgggccacctctgaagaggacatggcccaggatacaatcatctataccgacgagtccttcacccctgatctgaatatcttccaagacgtgcttcatagagatacactggtgaaagccttcctcgaccaggtgttccagctgaagcctggcctgagcctgaggtccacattcctcgctcagttcctgctcgtgctgcacagaaaggccctgacccttatcaagtacatcgaggatgacacccagaagggcaagaagccgttcaagtccctcagaaacctgaaaatcgacctggacctgacagccgagggagatctgaacatcatcatggctctggccgaaaagatcaagcccggcctgcattctttcatcttcggcagacctttttacaccagcgtgcaagagcgggacgtgctgatgacattctga

SEQ ID NO: 101 - DNA, 1446 bp, synthetic construct
  misc_feature 1..1446: note = Description of Artificial Sequence: Synthetic polynucleotide
  source 1..1446: mol_type = other DNA
atgagcaccctgtgccctccacctagccccgccgtggccaagacagagatcgccctttctggcaagtccccactgctggccgctaccttcgcctattgggacaacatcttgggccccagagtgcggcacatctgggcccctaagaccgagcaggtgctgctgagtgatggcgagatcaccttcctggctaatcacaccctgaacggcgagatcctgagaaacgccgagagcggcgccatcgacgtgaaattcttcgtgctgagcgagaaaggcgtgatcatcgtgtccctgatcttcgacggaaattggaacggcgacagaagcacctacggcctgagcatcatcctcccccagaccgagctgtccttctacctgcctctgcatagagtgtgcgtggaccgcctgacacacatcattagaaagggcagaatctggatgcacaaggaacggcaggagaacgtgcagaaaattatcctggaaggtacagagagaatggaagatcagggacagtctatcatccccatgctgaccggcgaagtgatccctgtgatggaactgctgtctagcatgaagtctcattctgtgcctgaggaaatcgacatcgccgacaccgtgctgaacgacgacgacatcggcgatagctgccacgagggcttcctgctgaacgccattagcagccacctgcagacctgcggatgtagcgtggtggtcggcagcagcgccgagaaggtgaacaagatcgtgcggacactgtgcctgttcctcacacctgctgaaagaaagtgcagcagactgtgtgaagccgaaagcagctttaagtacgagagcggcctgttcgtgcaaggcctgctgaaggacagcacaggctcttttgtgctgcctttcagacaggtgatgtacgccccttaccccaccacacacattgacgtggacgtgaacaccgtgaagcagatgcctccttgtcacgagcacatctacaaccagagaagatacatgagatctgagctgaccgccttttggcgggccaccagcgaagaggacatggcccaggataccatcatctacactgatgagagcttcacccctgatctgaacattttccaggacgtgctgcacagagataccctggtgaaggccttcctggaccaggtctttcagctgaaacctggactgagcctgcggtccacattcctggcccaatttctgctggtgctgcaccggaaggctctgactctgatcaagtatatcgaggacgatacacagaagggcaaaaagcccttcaagagcctgagaaatctgaagatcgatctggatctgacagccgagggcgacctgaatatcatcatggccctggcagaaaagattaagcctggcctgcacagcttcatcttcggccgtccattctacacctctgtgcaggagcgggacgttctcatgaccttctga

SEQ ID NO: 102 - DNA, 1446 bp, synthetic construct
  misc_feature 1..1446: note = Description of Artificial Sequence: Synthetic polynucleotide
  source 1..1446: mol_type = other DNA
atgagcaccctttgtcctcctccatctcctgccgtggccaagacagaaatcgccctgtccggcaagtcccctctgctggctgctacatttgcctactgggacaacatcctgggacctagagttagacacatctgggcccctaagaccgagcaggttctgctgagtgatggcgagataacattcctggccaaccacaccctgaatggagaaatcctgagaaacgccgagagcggcgccatcgatgtgaagttcttcgtgctgagcgagaagggcgtgatcattgtgtccctgatcttcgacggcaactggaacggcgatagatctacatacggcctgtccatcatcctgccccagaccgagctgagcttttacctgcctctgcacagagtttgtgtggacagactgactcacattatcagaaagggaagaatctggatgcacaaggaaagacaggagaacgtgcagaagattattctggaaggtacagagagaatggaagatcagggccagagcatcatccccatgctgaccggcgaggtgatccctgtgatggaactgctgagcagcatgaaaagccacagcgtgcccgaggaaatcgacatcgccgacacagtgctgaatgatgacgacatcggcgacagctgccacgagggcttcctgctgaacgctatcagctctcatctgcagacatgcggctgtagcgtcgtggtgggcagctccgccgagaaggtgaacaagatcgtgcggacactgtgcctgttcctcacccctgctgaacggaaatgctctagactctgcgaggccgagagcagcttcaagtacgagtccggcctcttcgtgcaaggcctgctgaaagacagtacaggcagcttcgtgctgcctttcagacaggtcatgtacgccccttaccccaccacccacatcgatgtggacgtgaacaccgtgaagcagatgcctccgtgccacgagcacatctacaaccagagaagatacatgcggtctgaactgacagccttttggcgggccaccagcgaagaggacatggcccaggacaccatcatctacaccgacgagtctttcacccctgacctgaatatctttcaggatgtgctgcacagagataccctggtcaaggccttcctggaccaggtgttccagctgaagcctggactgtctctgcggagcaccttcctggcccaatttcttctggtgctccaccggaaggccctgacactgatcaagtacatcgaggacgacacccagaaaggaaaaaagccgttcaagtccctgcggaacctgaagatcgacctggatctgaccgccgagggcgacctgaacatcatcatggccctggctgagaaaatcaagcctggcctgcacagcttcatcttcggcagacctttctacaccagcgtgcaggagcgggacgtgctgatgaccttctga

SEQ ID NO: 103 - DNA, 1446 bp, synthetic construct
  misc_feature 1..1446: note = Description of Artificial Sequence: Synthetic polynucleotide
  source 1..1446: mol_type = other DNA
atgagcacactgtgccccccaccttctccagccgtggccaagaccgagatcgccctttctggcaagagccctctgctggccgccacattcgcctactgggacaacatcctgggccctagagtgcggcacatctgggcccctaagaccgagcaggtgctgctgagtgatggcgaaataacattcctggctaatcacaccctcaacggagagatcctgagaaatgccgagagcggcgccatcgacgtcaagttcttcgtgctgtctgaaaagggcgtgatcatagtttctctgatcttcgacggcaactggaacggcgacagaagcacctacggcctgtccatcatcctgccccagacagaactgagcttttacctgcctctgcacagagtgtgcgtggaccggctgacccacatcattagaaagggcagaatctggatgcacaaggaacggcaggagaacgtgcagaagatcatcctggaagggaccgaaagaatggaagatcagggccagagcatcattcctatgctgacaggcgaggtgatccccgtgatggaactgctgagcagcatgaagtctcactctgtccccgaggaaatcgacatcgccgacactgtgctcaacgacgacgatatcggcgatagctgccacgagggatttctgctgaacgccatttctagccacctgcagacctgtggctgcagcgtggtcgtgggcagctccgccgagaaggtgaacaagatcgtgcggaccctgtgcctgtttctgacacctgctgaacggaagtgcagtagactgtgtgaagccgagagcagcttcaaatacgagagcggactgttcgttcaaggcctgctgaaggacagcaccggaagcttcgtgctgcctttcagacaggtgatgtacgccccttaccccacaacacacattgatgtcgatgtgaacacagtgaaacagatgcctccatgtcacgagcacatctacaaccagaggcggtacatgagaagcgagctgaccgccttttggcgggccaccagcgaggaagatatggcccaggacacaatcatctacactgatgagtcctttacccctgatctgaatatcttccaggacgtgctgcatagagacaccctggtgaaggccttcctggaccaggtgttccagctgaagcctggactcagcctgcggagcaccttcctcgctcagttcctgctcgtgctgcacagaaaggccctgaccctgatcaagtacatcgaggacgacacccagaaaggcaaaaagcccttcaagtccctcagaaacctgaaaatcgacctggacctgaccgccgaaggcgacctgaacatcatcatggccctggccgagaagatcaaacctggcctgcacagcttcatcttcggcagacctttctacaccagcgtgcaggagagagatgtgctgatgaccttttga

SEQ ID NO: 104 - DNA, 1446 bp, synthetic construct
  misc_feature 1..1446: note = Description of Artificial Sequence: Synthetic polynucleotide
  source 1..1446: mol_type = other DNA
atgagcaccctgtgccctccacctagccctgccgtggccaagacagagatcgcactgtccggcaagtccccactgctggccgccaccttcgcctactgggacaacatcctgggccctagagtgcggcacatttgggcccctaagaccgagcaggtgctgctgtctgatggcgagatcaccttcctggctaatcacaccctgaacggcgaaatcctgagaaatgccgagagcggcgccatcgacgtgaagttcttcgtgctgtctgagaagggcgtgatcatcgtgtccctgatcttcgacggcaactggaacggcgaccggagcacctacggcctgagcatcatcctgcctcagaccgaactgtccttttacctgcctctgcacagagtgtgcgtggacagactgacacacatcatcagaaagggcagaatctggatgcacaaggaaagacaggagaacgtgcagaagatcattctggaaggtacagaaagaatggaagatcagggccagagcatcattcctatgctgaccggcgaggtgatccccgtgatggaactgctgagcagcatgaaaagccacagcgtccccgaggaaatcgacatcgctgataccgtgctgaacgacgacgatatcggcgatagctgccacgagggcttcctgctgaacgccatcagcagccacctgcagacctgcggctgcagcgtggtcgtgggcagctccgccgagaaggtgaacaagatcgtgcggaccctgtgtctgttcctgacccctgctgagagaaagtgcagcagactgtgtgaagccgagtcctccttcaaatacgagagcggattgtttgtgcaaggactcctgaaggacagcacaggctctttcgtgctgcccttcagacaggtgatgtacgccccttaccccaccacacacattgacgtggacgtcaacacagtgaaacagatgcctccatgtcacgagcacatctacaaccagagacggtacatgagaagcgagctgaccgccttttggcgggccacaagcgaggaagatatggcccaagatacaatcatctatacagacgagtctttcacccctgatctgaatatctttcaggacgtcctgcaccgggacaccctggtgaaggccttcctggatcaggtgttccagctgaaacccggcctgtctctgcggtccaccttcctggcccagttcctgctggtcctgcatagaaaagccctgaccctgatcaagtacatcgaggacgacacgcagaaaggaaagaagcccttcaagagccttagaaacctgaagatcgacctggacctcacagccgaaggcgacctgaacatcatcatggctctggccgaaaaaatcaagcctggcctgcatagcttcatcttcggcagacctttctacacctctgtccaggagagagatgtgctgatgacattctga

SEQ ID NO: 105 - DNA, 1446 bp, synthetic construct
  misc_feature 1..1446: note = Description of Artificial Sequence: Synthetic polynucleotide
  source 1..1446: mol_type = other DNA
atgagcaccctctgtcctccccccagccctgctgtggccaagacagagatcgccctgtctggaaagtcccctctgctggctgctacattcgcctactgggacaacatcctgggccccagagtgcggcacatctgggcccctaagaccgagcaggtgctcctgagcgacggcgagatcaccttcctggctaatcacaccctgaacggcgagatcctgagaaatgccgaaagcggcgccatcgacgtgaagttcttcgtgctgtctgagaagggcgtgatcattgtgtccctgatcttcgacggcaactggaacggcgatagatctacatacggcctgagcatcatcctgcctcagaccgagctgtccttctacctgcctctgcacagagtgtgcgtggacagactgacacacatcattagaaagggcaggatctggatgcacaaggaaagacaggagaacgtgcagaagatcatcctggaagggaccgaaagaatggaagatcagggccagagcatcatccctatgctgaccggcgaagtgatccccgtgatggaactgctgagttccatgaaaagccactctgtgcccgaggaaatcgacatcgccgacaccgtgctgaacgacgacgacataggagatagctgccatgagggcttcctgctgaacgccatcagcagccacctgcagacctgcggttgtagcgtggtggtgggctctagcgccgagaaggtgaacaagatcgtgcggaccctgtgcctgttcctgacacctgccgaacgaaaatgctctagactgtgtgaagccgagagcagctttaagtacgagagcggcctgttcgtgcaaggcctgcttaaagacagcaccggcagcttcgttctgccattcagacaggtgatgtacgccccttaccctaccacccacattgacgtcgacgtgaacaccgtgaaacagatgcctccttgccacgagcacatctacaaccagagaagatacatgcggagcgagttgaccgccttctggcgggccaccagcgaggaagatatggcccaggacaccatcatctacaccgacgagagcttcacccctgacctgaacatctttcaggatgtgctgcatagagatacactggtgaaggcctttctcgaccaggttttccagctgaagcccggcctgagcctgcggagcacatttctggctcaatttctcctggtcctgcaccggaaagccctgacactgatcaagtacatcgaggatgacacccagaaaggcaaaaagcccttcaagagcctgagaaacctgaagatcgacctggacctgaccgccgagggcgaccttaatatcatcatggccctggctgaaaagattaagcctggcctgcacagcttcatcttcggcagacctttctatacaagcgtgcaggagcgggacgtgctgatgacattctga

SEQ ID NO: 106 - DNA, 1446 bp, synthetic construct
  misc_feature 1..1446: note = Description of Artificial Sequence: Synthetic polynucleotide
  source 1..1446: mol_type = other DNA
atgagcacactgtgtcctccaccatctcctgccgtggccaagaccgagatcgccctgagcggaaaaagccccctgctggccgctaccttcgcctactgggacaacatcctgggccctagagtgcggcacatctgggcccctaagacagagcaggtgctcctgagtgatggcgagataacattcctggctaatcacaccctgaatggcgaaatcctgagaaacgccgaaagtggcgccattgacgtgaagttcttcgtgctgtccgagaagggcgtgatcatcgtgtccctgatcttcgacggcaactggaacggcgatagaagcacctacggcctgtctatcatcctgcctcagaccgagctgagcttctacctgcctctgcacagagtgtgcgtggacagactgacacacatcattagaaagggcagaatctggatgcacaaggaacggcaggagaacgtgcagaaaatcatcctggaagggaccgaaaggatggaagatcagggccagagcatcatccccatgctgactggagaggtgatccctgttatggaactgctgagcagcatgaagagccacagcgtgcccgaagagattgacatcgccgacaccgtgctgaacgacgacgacataggagattcatgccacgaaggattcctgctcaacgccatcagcagccacctgcagacatgcggctgctctgtggtcgtgggcagcagcgccgagaaagtgaacaagatcgtgcggaccctctgtctgtttctcacacccgctgagcggaagtgcagcagactgtgcgaggccgagtctagctttaagtacgagagcggcctgttcgtgcaaggcctgctgaaggactctaccggctcctttgtgctcccttttagacaggtgatgtacgccccttaccccaccacccacattgatgtggacgtcaacaccgtgaaacagatgcctccttgccacgagcacatctacaaccagagacggtacatgcggagcgagctgaccgccttctggcgggccacctccgaggaagatatggcccaggacaccatcatctatactgatgagtctttcacccctgatctgaacatctttcaggatgtgctgcaccgggacaccctggtgaaggctttcctcgaccaggtgttccagctgaaacctggcctcagcctcagaagcacattcctggcccagttcctgctcgtgctccatagaaaggccctgacactgatcaagtacatcgaggatgatacacagaagggcaagaagcctttcaagtccctgcggaacctgaagatcgacctggacctgacagccgaaggcgacctgaacatcattatggccctggccgagaagatcaagcccggcctgcattctttcatcttcggcagacctttctacaccagcgtgcaggagagagatgttctgatgaccttctga

SEQ ID NO: 107 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttcagtgtcagcctttcatacgttttggccactgactgacgtatgaaactgacactgaagacacaaggcctgttactagcactcacatggaacaaatggcccagatctggccgcactcgagatatctagaacccagctttcttgtacaaagtggtcccc

SEQ ID NO: 108 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctatcagaagcactttagtcctggttttggccactgactgaccaggactagtgcttctgatgacacaaggcctgttactagcactcacatggaacaaatggcccagatctggccgcactcgagatatctagaacccagctttcttgtacaaagtggtcccc

SEQ ID NO: 109 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttgaatcagaagcactttagtgttttggccactgactgacactaaagtttctgattcaagacacaaggcctgttactagcactcacatggaacaaatggcccagatctggccgcactcgagatatctagaacccagctttcttgtacaaagtggtcccc

SEQ ID NO: 110 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttaacctaagagccttaatggcgttttggccactgactgacgccattaactcttaggttagacacaaggcctgttactagcactcacatggaacaaatggcccagatctggccgcactcgagatatctagaacccagctttcttgtacaaagtggtcccc

SEQ ID NO: 111 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttcatgatggagtatcagaggcgttttggccactgactgacgcctctgactccatcatgagacacaaggcctgttactagcactcacatggaacaaatggcccagatctggccgcactcgagatatctagaacccagctttcttgtacaaagtggtcccc

SEQ ID NO: 112 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttaatagtacctaatgtgtagggttttggccactgactgaccctacacaaggtactattagacacaaggcctgttactagcactcacatggaacaaatggcccagatctggccgcactcgagatatctagaacccagctttcttgtacaaagtggtcccc

SEQ ID NO: 113 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaaagctaacagaatcctttcagttttggccactgactgactgaaaggactgttagctttgacacaaggcctgttactagcactcacatggaacaaatggcccagatctggccgcactcgagatatctagaacccagctttcttgtacaaagtggtcccc

SEQ ID NO: 114 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctcattaaagctaacagaatcctgttttggccactgactgacaggattcttagctttaatggacacaaggcctgttactagcactcacatggaacaaatggcccagatctggccgcactcgagatatctagaacccagctttcttgtacaaagtggtcccc

SEQ ID NO: 115 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctataacagactgtctacttagagttttggccactgactgactctaagtacagtctgttatgacacaaggcctgttactagcactcacatggaacaaatggcccagatctggccgcactcgagatatctagaacccagctttcttgtacaaagtggtcccc

SEQ ID NO: 116 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaataacagactgtctacttaggttttggccactgactgacctaagtagagtctgttattgacacaaggcctgttactagcactcacatggaacaaatggcccagatctggccgcactcgagatatctagaacccagctttcttgtacaaagtggtcccc

SEQ ID NO: 117 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttgaagtttatggtagtgcacagttttggccactgactgactgtgcactcataaacttcagacacaaggcctgttactagcactcacatggaacaaatggcccagatctggccgcactcgagatatctagaacccagctttcttgtacaaagtggtcccc

SEQ ID NO: 118 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttcttctgaagtttatggtaggttttggccactgactgacctaccatacttcagaagaagacacaaggcctgttactagcactcacatggaacaaatggcccagatctggccgcactcgagatatctagaacccagctttcttgtacaaagtggtcccc

SEQ ID NO: 119 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttaacctgcttgaccagctttgttttggccactgactgacaaagctggaagcaggttaagacacaaggcctgttactagcactcacatggaacaaatggcccagatctggccgcactcgagatatctagaacccagctttcttgtacaaagtggtcccc

SEQ ID NO: 120 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttgtttaacctgcttgaccagcgttttggccactgactgacgctggtcacaggttaaacagacacaaggcctgttactagcactcacatggaacaaatggcccagatctggccgcactcgagatatctagaacccagctttcttgtacaaagtggtcccc

SEQ ID NO: 121 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaaattgtttaacctgcttgacgttttggccactgactgacgtcaagcattaaacaatttgacacaaggcctgttactagcactcacatggaacaaatggcccagatctggccgcactcgagatatctagaacccagctttcttgtacaaagtggtcccc

SEQ ID NO: 122 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctatttaggttagtctcctgattgttttggccactgactgacaatcaggactaacctaaatgacacaaggcctgttactagcactcacatggaacaaatggcccagatctggccgcactcgagatatctagaacccagctttcttgtacaaagtggtcccc

SEQ ID NO: 123 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctacctttaggaaactattcttggttttggccactgactgaccaagaatattcctaaaggtgacacaaggcctgttactagcactcacatggaacaaatggcccagatctggccgcactcgagatatctagaacccagctttcttgtacaaagtggtcccc

SEQ ID NO: 124 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaagagatacctttaggaaactgttttggccactgactgacagtttcctaggtatctcttgacacaaggcctgttactagcactcacatggaacaaatggcccagatctggccgcactcgagatatctagaacccagctttcttgtacaaagtggtcccc

SEQ ID NO: 125 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctcaaagtagtaaccattaatgggttttggccactgactgacccattaatttactactttggacacaaggcctgttactagcactcacatggaacaaatggcccagatctggccgcactcgagatatctagaacccagctttcttgtacaaagtggtcccc

SEQ ID NO: 126 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttcacatacagtattagccacgttttggccactgactgacgtggctaactgtatgtgaagacacaaggcctgttactagcactcacatggaacaaatggcccagatctggccgcactcgagatatctagaacccagctttcttgtacaaagtggtcccc

SEQ ID NO: 127 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttaaggttcgcacacgctattgttttggccactgactgacaatagcgtgcgaaccttaagacacaaggcctgttactagcactcacatggaacaaatggcccagatctggccgcactcgagatatctagaacccagctttcttgtacaaagtggtcccc

SEQ ID NO: 128 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctattaaggttcgcacacgctatgttttggccactgactgacatagcgtgcgaaccttaatgacacaaggcctgttactagcactcacatggaacaaatggcccagatctggccgcactcgagatatctagaacccagctttcttgtacaaagtggtcccc

SEQ ID NO: 129 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttattaaggttcgcacacgctagttttggccactgactgactagcgtgtgaaccttaatagacacaaggcctgttactagcactcacatggaacaaatggcccagatctggccgcactcgagatatctagaacccagctttcttgtacaaagtggtcccc

SEQ ID NO: 130 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaactcatccacatattgcaacgttttggccactgactgacgttgcaatgtggatgagttgacacaaggcctgttactagcactcacatggaacaaatggcccagatctggccgcactcgagatatctagaacccagctttcttgtacaaagtggtcccc

SEQ ID NO: 131 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctagtaagtggaatctatacaccgttttggccactgactgacggtgtatattccacttactgacacaaggcctgttactagcactcacatggaacaaatggcccagatctggccgcactcgagatatctagaacccagctttcttgtacaaagtggtcccc

SEQ ID NO: 132 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaatgctactcatctgtagtaagttttggccactgactgacttactacatgagtagcattgacacaaggcctgttactagcactcacatggaacaaatggcccagatctggccgcactcgagatatctagaacccagctttcttgtacaaagtggtcccc

SEQ ID NO: 133 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttgtagtaagtgccatctcacagttttggccactgactgactgtgagatcacttactacagacacaaggcctgttactagcactcacatggaacaaatggcccagatctggccgcactcgagatatctagaacccagctttcttgtacaaagtggtcccc

SEQ ID NO: 134 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttactcactgtagtaagtgccagttttggccactgactgactggcactttacagtgagtagacacaaggcctgttactagcactcacatggaacaaatggcccagatctggccgcactcgagatatctagaacccagctttcttgtacaaagtggtcccc

SEQ ID NO: 135 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaaatgctactcactgtagtaagttttggccactgactgacttactacagagtagcatttgacacaaggcctgttactagcactcacatggaacaaatggcccagatctggccgcactcgagatatctagaacccagctttcttgtacaaagtggtcccc

SEQ ID NO: 136 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttaaatgctactcactgtagtgttttggccactgactgacactacagtgtagcatttaagacacaaggcctgttactagcactcacatggaacaaatggcccagatctggccgcactcgagatatctagaacccagctttcttgtacaaagtggtcccc

SEQ ID NO: 137 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaaacttagcactctactaacagttttggccactgactgactgttagtagtgctaagtttgacacaaggcctgttactagcactcacatggaacaaatggcccagatctggccgcactcgagatatctagaacccagctttcttgtacaaagtggtcccc

SEQ ID NO: 138 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctataccaatcagggaagagatggttttggccactgactgaccatctcttctgattggtatgacacaaggcctgttactagcactcacatggaacaaatggcccagatctggccgcactcgagatatctagaacccagctttcttgtacaaagtggtcccc

SEQ ID NO: 139 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctctaaataccaatcagggaagagttttggccactgactgactcttccctttggtatttaggacacaaggcctgttactagcactcacatggaacaaatggcccagatctggccgcactcgagatatctagaacccagctttcttgtacaaagtggtcccc

SEQ ID NO: 140 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttaaacagcatggttacaagtagttttggccactgactgactacttgtacatgctgtttagacacaaggcctgttactagcactcacatggaacaaatggcccagatctggccgcactcgagatatctagaacccagctttcttgtacaaagtggtcccc

SEQ ID NO: 141 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctataaacagcatggttacaagtgttttggccactgactgacacttgtaaatgctgtttatgacacaaggcctgttactagcactcacatggaacaaatggcccagatctggccgcactcgagatatctagaacccagctttcttgtacaaagtggtcccc

SEQ ID NO: 142 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttctggtactgtaaacagttcgttttggccactgactgacgaactgttcagtaccagaagacacaaggcctgttactagcactcacatggaacaaatggcccagatctggccgcactcgagatatctagaacccagctttcttgtacaaagtggtcccc

SEQ ID NO: 143 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctatgaacttcaccttccagtctgttttggccactgactgacagactggagtgaagttcatgacacaaggcctgttactagcactcacatggaacaaatggcccagatctggccgcactcgagatatctagaacccagctttcttgtacaaagtggtcccc

SEQ ID NO: 144 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttagatagttcccaggaggacgttttggccactgactgacgtcctcctgaactatctaagacacaaggcctgttactagcactcacatggaacaaatggcccagatctggccgcactcgagatatctagaacccagctttcttgtacaaagtggtcccc

SEQ ID NO: 145 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaacaaagtaaaccaaggaggagttttggccactgactgactcctcctttttactttgttgacacaaggcctgttactagcactcacatggaacaaatggcccagatctggccgcactcgagatatctagaacccagctttcttgtacaaagtggtcccc

SEQ ID NO: 146 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaaacaaagtaaaccaaggagggttttggccactgactgaccctccttgttactttgtttgacacaaggcctgttactagcactcacatggaacaaatggcccagatctggccgcactcgagatatctagaacccagctttcttgtacaaagtggtcccc

SEQ ID NO: 147 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttagtatgtatgacaaagtcctgttttggccactgactgacaggactttcatacatactagacacaaggcctgttactagcactcacatggaacaaatggcccagatctggccgcactcgagatatctagaacccagctttcttgtacaaagtggtcccc

SEQ ID NO: 148 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttgctaaagtggctaatactggttttggccactgactgaccagtattacactttagcaagacacaaggcctgttactagcactcacatggaacaaatggcccagatctggccgcactcgagatatctagaacccagctttcttgtacaaagtggtcccc

SEQ ID NO: 149 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaaacgtcctcaacaaatgattgttttggccactgactgacaatcattttgaggacgtttgacacaaggcctgttactagcactcacatggaacaaatggcccagatctggccgcactcgagatatctagaacccagctttcttgtacaaagtggtcccc

SEQ ID NO: 150 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctagaatcaggagactaacctaagttttggccactgactgacttaggttactcctgattctgacacaaggcctgttactagcactcacatggaacaaatggcccagatctggccgcactcgagatatctagaacccagctttcttgtacaaagtggtcccc

SEQ ID NO: 151 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttcatttccgagaatcaagacgttttggccactgactgacgtcttgattcggaaatgaagacacaaggcctgttactagcactcacatggaacaaatggcccagatctggccgcactcgagatatctagaacccagctttcttgtacaaagtggtcccc

SEQ ID NO: 152 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttagtctggctgtaacatagtggttttggccactgactgaccactatgtcagccagactagacacaaggcctgttactagcactcacatggaacaaatggcccagatctggccgcactcgagatatctagaacccagctttcttgtacaaagtggtcccc

SEQ ID NO: 153 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttagtctggctgtaacatagtgttttggccactgactgacactatgttagccagactaagacacaaggcctgttactagcactcacatggaacaaatggcccagatctggccgcactcgagatatctagaacccagctttcttgtacaaagtggtcccc

SEQ ID NO: 154 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctataggtgagcataagatggtagttttggccactgactgactaccatcttgctcacctatgacacaaggcctgttactagcactcacatggaacaaatggcccagatctggccgcactcgagatatctagaacccagctttcttgtacaaagtggtcccc

SEQ ID NO: 155 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaatctaagtagacagtctgttgttttggccactgactgacaacagactctacttagattgacacaaggcctgttactagcactcacatggaacaaatggcccagatctggccgcactcgagatatctagaacccagctttcttgtacaaagtggtcccc

SEQ ID NO: 156 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctagaacaatctaagtagacagtgttttggccactgactgacactgtctatagattgttctgacacaaggcctgttactagcactcacatggaacaaatggcccagatctggccgcactcgagatatctagaacccagctttcttgtacaaagtggtcccc

SEQ ID NO: 157 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttaagtactaaactccactgcgttttggccactgactgacgcagtggattagtacttaagacacaaggcctgttactagcactcacatggaacaaatggcccagatctggccgcactcgagatatctagaacccagctttcttgtacaaagtggtcccc

SEQ ID NO: 158 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaactcttaagtactaaactccgttttggccactgactgacggagtttaacttaagagttgacacaaggcctgttactagcactcacatggaacaaatggcccagatctggccgcactcgagatatctagaacccagctttcttgtacaaagtggtcccc

SEQ ID NO: 159 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaattcaggcaccttgcccacggttttggccactgactgaccgtgggcagtgcctgaattgacacaaggcctgttactagcactcacatggaacaaatggcccagatctggccgcactcgagatatctagaacccagctttcttgtacaaagtggtcccc

SEQ ID NO: 160 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctagagaattcaggcaccttgccgttttggccactgactgacggcaaggtctgaattctctgacacaaggcctgttactagcactcacatggaacaaatggcccagatctggccgcactcgagatatctagaacccagctttcttgtacaaagtggtcccc

SEQ ID NO: 161 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctataacaaccctacacattagggttttggccactgactgaccctaatgtagggttgttatgacacaaggcctgttactagcactcacatggaacaaatggcccagatctggccgcactcgagatatctagaacccagctttcttgtacaaagtggtcccc

SEQ ID NO: 162 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttctgattcaagccattaagggttttggccactgactgacccttaatgttgaatcagaagacacaaggcctgttactagcactcacatggaacaaatggcccagatctggccgcactcgagatatctagaacccagctttcttgtacaaagtggtcccc

SEQ ID NO: 163 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttacaggactaaagtgcttctggttttggccactgactgaccagaagcattagtcctgtagacacaaggcctgttactagcactcacatggaacaaatggcccagatctggccgcactcgagatatctagaacccagctttcttgtacaaagtggtcccc

SEQ ID NO: 164 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaacagatacaggactaaagtggttttggccactgactgaccactttagctgtatctgttgacacaaggcctgttactagcactcacatggaacaaatggcccagatctggccgcactcgagatatctagaacccagctttcttgtacaaagtggtcccc

SEQ ID NO: 165 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctatgaaaggctgacactgaacagttttggccactgactgactgttcagtcagcctttcatgacacaaggcctgttactagcactcacatggaacaaatggcccagatctggccgcactcgagatatctagaacccagctttcttgtacaaagtggtcccc

SEQ ID NO: 166 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaatgatgtatgaaaggctgacgttttggccactgactgacgtcagcctcatacatcattgacacaaggcctgttactagcactcacatggaacaaatggcccagatctggccgcactcgagatatctagaacccagctttcttgtacaaagtggtcccc

SEQ ID NO: 167 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttgagatggcacttactacagtgttttggccactgactgacactgtagtgtgccatctcagacacaaggcctgttactagcactcacatggaacaaatggcccagatctggccgcactcgagatatctagaacccagctttcttgtacaaagtggtcccc

SEQ ID NO: 168 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctatgagtagcatttacaccactgttttggccactgactgacagtggtgtatgctactcatgacacaaggcctgttactagcactcacatggaacaaatggcccagatctggccgcactcgagatatctagaacccagctttcttgtacaaagtggtcccc

SEQ ID NO: 169 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttatagattccacttactacaggttttggccactgactgacctgtagtatggaatctatagacacaaggcctgttactagcactcacatggaacaaatggcccagatctggccgcactcgagatatctagaacccagctttcttgtacaaagtggtcccc

SEQ ID NO: 170 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaaacgtaccattctgtttgatgttttggccactgactgacatcaaacaatggtacgtttgacacaaggcctgttactagcactcacatggaacaaatggcccagatctggccgcactcgagatatctagaacccagctttcttgtacaaagtggtcccc

SEQ ID NO: 171 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttttaccgtaagacactgttaagttttggccactgactgacttaacagtcttacggtaaagacacaaggcctgttactagcactcacatggaacaaatggcccagatctggccgcactcgagatatctagaacccagctttcttgtacaaagtggtcccc

SEQ ID NO: 172 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaatagcgtgtgcgaaccttaagttttggccactgactgacttaaggttcacacgctattgacacaaggcctgttactagcactcacatggaacaaatggcccagatctggccgcactcgagatatctagaacccagctttcttgtacaaagtggtcccc

SEQ ID NO: 173 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttaagacccgctctggaggaggttttggccactgactgacctcctccagcgggtcttaagacacaaggcctgttactagcactcacatggaacaaatggcccagatctggccgcactcgagatatctagaacccagctttcttgtacaaagtggtcccc

SEQ ID NO: 174 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttttatcttaagacccgctctggttttggccactgactgaccagagcggcttaagataaagacacaaggcctgttactagcactcacatggaacaaatggcccagatctggccgcactcgagatatctagaacccagctttcttgtacaaagtggtcccc

SEQ ID NO: 175 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttttctcacgaggctagcgaaagttttggccactgactgactttcgctactcgtgagaaagacacaaggcctgttactagcactcacatggaacaaatggcccagatctggccgcactcgagatatctagaacccagctttcttgtacaaagtggtcccc

SEQ ID NO: 176 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttccagagcttgctacaggctgttttggccactgactgacagcctgtaaagctctggaagacacaaggcctgttactagcactcacatggaacaaatggcccagatctggccgcactcgagatatctagaacccagctttcttgtacaaagtggtcccc

SEQ ID NO: 177 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttgtactatcagcatgtagcaggttttggccactgactgacctgctacactgatagtacagacacaaggcctgttactagcactcacatggaacaaatggcccagatctggccgcactcgagatatctagaacccagctttcttgtacaaagtggtcccc

SEQ ID NO: 178 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttcagatgtactatcagcatggttttggccactgactgaccatgctgagtacatctgaagacacaaggcctgttactagcactcacatggaacaaatggcccagatctggccgcactcgagatatctagaacccagctttcttgtacaaagtggtcccc

SEQ ID NO: 179 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaattaacgtagaatagaacccgttttggccactgactgacgggttctactacgttaattgacacaaggcctgttactagcactcacatggaacaaatggcccagatctggccgcactcgagatatctagaacccagctttcttgtacaaagtggtcccc

SEQ ID NO: 180 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttaaaccgtccactttccacaagttttggccactgactgacttgtggaatggacggtttagacacaaggcctgttactagcactcacatggaacaaatggcccagatctggccgcactcgagatatctagaacccagctttcttgtacaaagtggtcccc

SEQ ID NO: 181 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttgcactggcaggatcatagctgttttggccactgactgacagctatgactgccagtgcagacacaaggcctgttactagcactcacatggaacaaatggcccagatctggccgcactcgagatatctagaacccagctttcttgtacaaagtggtcccc

SEQ ID NO: 182 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctagaggtttcccaatacactttgttttggccactgactgacaaagtgtagggaaacctctgacacaaggcctgttactagcactcacatggaacaaatggcccagatctggccgcactcgagatatctagaacccagctttcttgtacaaagtggtcccc

SEQ ID NO: 183 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctttcaaattgagtgagacggtggttttggccactgactgaccaccgtctctcaatttgaagacacaaggcctgttactagcactcacatggaacaaatggcccagatctggccgcactcgagatatctagaacccagctttcttgtacaaagtggtcccc

SEQ ID NO: 184 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctccaagattcaaattgagtgaggttttggccactgactgacctcactcattgaatcttgggacacaaggcctgttactagcactcacatggaacaaatggcccagatctggccgcactcgagatatctagaacccagctttcttgtacaaagtggtcccc

SEQ ID NO: 185 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgctaatacttgaagtcatcgtcttgttttggccactgactgacaagacgatcttcaagtattgacacaaggcctgttactagcactcacatggaacaaatggcccagatctggccgcactcgagatatctagaacccagctttcttgtacaaagtggtcccc

SEQ ID NO: 186 - DNA, 246 bp, synthetic construct
  misc_feature 1..246: note = Description of Artificial Sequence: Synthetic polynucleotide
  source 1..246: mol_type = other DNA
ggggacaactttgtatacaaaagttgtattaaagggaggtagtgagtcgaccagtggatcctggaggcttgctgaaggctgtatgcttgaaatggtaatgacactactgttttggccactgactgacagtagtgtttaccatttcagacacaaggcctgttactagcactcacatggaacaaatggcccagatctggccgcactcgagatatctagaacccagctttcttgtacaaagtggtcccc

SEQ ID NO: 187 - DNA, 1714 bp, synthetic construct
  misc_feature 1..1714: note = Description of Artificial Sequence: Synthetic polynucleotide
  source 1..1714: mol_type = other DNA
atgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgaccaccaccaccaccaccacgagcagaagctgatctccgaggaggacctgtaacacccaacttttctatacaaagttgtccccggggacaacttttctatacaaagttgtagtatccaaggtagtggactagtgtgacgctgctgacccctttctttcccttctgcagaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactacccctacgacgtgcccgactacgcctaa

SEQ ID NO: 188 - DNA, 771 bp, synthetic construct
  misc_feature 1..771: note = Description of Artificial Sequence: Synthetic polynucleotide
  source 1..771: mol_type = other DNA
atgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcgtaagtcgactcgttggatccccactacagccgatactcaagcttgacgaattcgaccaccaccaccaccaccacgagcagaagctgatctccgaggaggacctgtaa

SEQ ID NO: 189 - DNA, 1491 bp, synthetic construct
  misc_feature 1..1491: note = Description of Artificial Sequence: Synthetic polynucleotide
  source 1..1491: mol_type = other DNA
atgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgacttttcaccaccaccaccaccactacccctacgacgtgcccgactacgcctaa

SEQ ID NO: 190 - DNA, 1446 bp, synthetic construct; identical to SEQ ID NO: 91
  misc_feature 1..1446: note = Description of Artificial Sequence: Synthetic polynucleotide
  source 1..1446: mol_type = other DNA

(19) United States
(12) Patent Application Publication
(10) Pub. No.: US 2024/0067984 A1
(43) Pub. Date: Feb. 29, 2024

(54) TRIPLE FUNCTION ADENO-ASSOCIATED VIRUS (AAV)VECTORS FOR THE TREATMENT OF C9ORF72 ASSOCIATED DISEASES

(71) Applicant: Applied Genetic Technologies Corporation, Alachua, FL (US)

(72) Inventors: Peixin Zhu, Lexington, MA (US); Xijia Wang, Alachua, FL (US); Steven Pennock, Leominster, MA (US); Mark Shearman, Alachua, FL (US)

(21) Appl. No.: 18/138,361

(22) Filed: Apr. 24, 2023

Related U.S. Application Data
(63) Continuation of application No. 17/077,682, filed on Oct. 22, 2020, now abandoned.
(60) Provisional application No. 62/924,351, filed on Oct. 22, 2019.

(51) Int. Cl. C12N 15/86; C07K 14/47; C12N 15/113; C12N 7/00

(52) U.S. Cl. CPC C12N 15/86; C07K 14/47; C12N 15/113; C12N 7/00; C12N 2750/14143; C12N 2310/141

(57) ABSTRACT

The present disclosure provides isolated promoters, transgene expression cassettes, vectors, kits, and methods for treatment of C9ORF72 associated diseases, including ALS and FTD.

### CROSS-REFERENCE TO RELATED APPLICATIONS

This application claims benefit under 35 U.S.C. § 119(e) of U.S. Provisional Application No. 62/924,351 filed Oct. 22, 2019, the contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 6, 2023, is named 119561-02003_SL.txt and is 693,877 bytes in size.

### FIELD OF THE INVENTION

The present invention relates to the field of gene therapy, including AAV vectors for expressing an isolated polynucleotides in a subject or cell. The disclosure also relates to nucleic acid constructs, promoters, vectors, and host cells including the polynucleotides as well as methods of delivering exogenous DNA sequences to a target cell, tissue, organ or organism, and methods for use in the treatment or prevention of c9orf72 associated diseases or disorders, such as amyotrophic lateral sclerosis (ALS) and frontotemporal lobar degeneration (FTLD).

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g., underexpression or overexpression, that can result in a disorder, disease, malignancy, etc. For example, a disease or disorder caused by a defective gene might be treated, prevented or ameliorated by delivery of a corrective genetic material to a patient, or might be treated, prevented or ameliorated by altering or silencing a defective gene, e.g., with a corrective genetic material to a patient resulting in the therapeutic expression of the genetic material within the patient.

The basis of gene therapy is to supply a transcription cassette with an active gene product (sometimes referred to as a transgene or a therapeutic nucleic acid), e.g., that can result in a positive gain-of-function effect, a negative loss-of-function effect, or another outcome. Such outcomes can be attributed to expression of a therapeutic protein such as an antibody, a functional enzyme, or a fusion protein. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors.

Adeno-associated viruses (AAV) belong to the Parvoviridae family and more specifically constitute the dependoparvovirus genus. Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes.

Amyotrophic lateral sclerosis (ALS) and frontotemporal lobar degeneration (FTLD) are severe neurodegenerative diseases with no effective treatment. ALS is a fatal neurodegenerative disease characterized clinically by progressive paralysis leading to death from respiratory failure, typically within two to three years of symptom onset (Rowland and Schneider, N. Engl. J. Med., 2001, 344, 1688-1700). ALS is the third most common neurodegenerative disease in the Western world (Hirtz et al., Neurology, 2007, 68, 326-337), and there are currently no effective therapies. Approximately 10% of cases are familial in nature, whereas the bulk of patients diagnosed with the disease are classified as sporadic as they appear to occur randomly throughout the population (Chio et al., Neurology, 2008, 70, 533-537). Some patients may also develop frontotemporal dementia. Frontotemporal dementia (FTD) is a group of related conditions resulting from the progressive degeneration of the temporal and frontal lobes of the brain. Depending on the affected regions, FTD patients suffer from dementia, behavioral abnormalities, language impairment and personality changes.

A strong genetic link and evidence from multiple families has been reported with autosomal dominant FTD and ALS. There is growing recognition, based on clinical, genetic, and epidemiological data, that ALS and FTD represent an overlapping continuum of disease, characterized pathologically by the presence of TDP-43 positive inclusions throughout the central nervous system (Lillo and Hodges, J. Clin. Neurosci., 2009, 16, 1131-1135; Neumann et al., Science, 2006, 314, 130-133). A mutation in the non-coding region of the C9orf72 gene has been identified as the most common genetic cause of both ALS and FTD (DeJesus-Hernandez et al., Neuron. 2011 Oct. 20; 72(2):245-56; Renton et al., Neuron. 2011 Oct. 20; 72(2):257-68). Two major mature mRNA transcript isoforms of c9orf72 are expressed, v1 & v2, with proposed distinct intracellular functions. v1 regulates Stress Granule assembly in response to cellular stress, while v2 does not appear to participate in stress granule assembly or regulation. Mutation carriers have a GGGGCC hexanucleotide repeat expansion either in the first intron or the promoter region, depending on the isoform of the c9orf72 transcript (Beck et al., Am J Hum Genet. 2013 Mar. 7; 92(3):345-53). Patients typically have several hundred or thousand repeats, whereas healthy controls show <33 repeats (Beck et al., 2013; van der Zee et al., Hum Mutat. 2013 February; 34(2):363-73).

In addition to the common TDP-43 aggregates in FTD and ALS, C9orf72 mutation carriers have abundant star-shaped, TDP-43-negative neuronal cytoplasmic inclusions (NCI) particularly in the cerebellum, hippocampus and frontal neocortex that stain positive for markers of the proteasome system (UPS) such as p62 or ubiquitin (Al Sarraj et al., Acta Neuropathol. 2011 December; 122(6):691-702). These TDP-43-negative inclusions contain dipeptide repeat proteins (DPR) that are translated ATG-independent from both sense and antisense transcripts of the C9orf72 repeat in all reading frames (Ash et al., Neuron. 2013 Feb. 20; 77(4):639-46; Gendron et al., Acta Neuropathol. 2013 December; 126(6):829-44; Mann et al., Acta Neuropathol Commun. 2013 Oct. 14; 10:68).

Although advances have been made in recent years regarding diagnostic criteria, clinical assessment instruments, neuropsychological tests, cerebrospinal fluid biomarkers, and brain imaging techniques, to date, there is no curative treatment for ALS or FTD. The present disclosure addresses the need for effective treatment of neurodegenerative diseases, such as ALS and FTD.

### SUMMARY OF THE INVENTION

The present disclosure describes, in part, triple function AAV vectors and their use in treating a c9orf72 associated disease, an in particular a c9orf72 hexanucleotide repeat expansion associated disease. The triple function of the AAV vectors described herein comprises c9orf72 gene supplementation, knock-down of c9orf72 sense transcripts and knock-down of c9orf72 anti-sense transcripts.

According to a first aspect, the disclosure provides a nucleic acid encoding a C9ORF72 protein, wherein the nucleic acid sequence is codon optimized. According to some embodiments, the nucleic acid sequence is codon optimized to avoid siRNA knockdown.

According to some embodiments, the codon optimized sequence is selected from a nucleic acid sequence set forth in Table 2. According to some embodiments, the codon optimized sequence is selected from a nucleic acid sequence selected from any one of SEQ ID NOs 21-52 and 100-106. According to some embodiments, the codon optimized sequence a nucleic acid sequence that is at least 85% identical, at least 90% identical, at least 95% identical, at least 96% identical, at least 97% identical, at least 98% identical, or at least 99% identical to any one of SEQ ID NOs 21-52 and 100-106.

According to another aspect, the disclosure provides a transgene expression cassette comprising a promoter; and the nucleic acid of any of the aspects and embodiments herein.

According to another aspect, the disclosure provides a transgene expression cassette comprising a promoter; the nucleic acid of any of the aspects and embodiments herein; a c9orf72 sense transcript specific inhibitor; and a c9orf72 antisense transcript specific inhibitor. According to some embodiments, the transgene expression cassette further comprises a c9orf72 sense transcript specific inhibitor. According to some embodiments, the nucleic acid is a microRNA (miRNA). According to some embodiments, the sense transcript inhibitor is selected from an miRNA set forth in Table 4. According to some embodiments, the antisense transcript inhibitor is selected from an miRNA set forth in Table 3. According to some embodiments, the c9orf72 sense transcript specific inhibitor is any of a nucleic acid, aptamer, antibody, peptide, or small molecule. According to some embodiments, the nucleic acid is a single-stranded nucleic acid or a double-stranded nucleic acid. According to some embodiments, the nucleic acid is a siRNA. According to some embodiments, the c9orf72 sense transcript inhibitor is an antisense compound. According to some embodiments, the antisense compound is an antisense oligonucleotide. According to some embodiments, the antisense compound is a modified oligonucleotide. According to some embodiments, the modified oligonucleotide has a nucleobase sequence that is at least 80%, at least 85%, at least 90%, at least 91%, at least 92%, at least 93%, at least 94%, at least 95%, at least 96%, at least 97%, at least 98%, at least 99%, or 100% complementary to a c9orf72 sense transcript. According to some embodiments, the transgene expression cassette further comprises a c9orf72 antisense transcript specific inhibitor. According to some embodiments, the c9orf72 antisense transcript specific inhibitor is an antisense compound. According to some embodiments, the c9orf72 antisense transcript specific antisense compound is an antisense oligonucleotide. According to some embodiments, the antisense oligonucleotide has a nucleobase sequence that is at least 80%, at least 85%, at least 90%, at least 91%, at least 92%, at least 93%, at least 94%, at least 95%, at least 96%, at least 97%, at least 98%, at least 99%, or 100% complementary to a c9orf72 antisense transcript. According to some embodiments, the antisense oligonucleotide is a modified antisense oligonucleotide. According to some embodiments, the antisense oligonucleotide is a gapmer. According to some embodiments, the transgene expression cassette further comprises two inverted terminal repeats (ITRs). According to some embodiments, the transgene expression cassette further comprises minimal regulatory elements (MRE). According to some embodiments, the promoter is specific for expression in neurons. According to some embodiments, the promoter is human Synapsin 1 (hSyn) promoter. According to some embodiments, the nucleic acid is a human nucleic acid.

According to other aspects, the disclosure provides a nucleic acid vector comprising the expression cassette of any of the aspects and embodiments herein. According to some embodiments, the vector is an adeno-associated viral (AAV) vector. According to some embodiments, the serotype of the capsid sequence and the serotype of the ITRs of said AAV vector are independently selected from the group consisting of AAV1, AAV2, AAV3, AAV4, AAV5, AAV6, AAV7, AAV8, AAV9, AAV10, AAV11, and AAV12. According to some embodiments, the capsid sequence is a mutant capsid sequence.

According to some embodiments, the vector comprises SEQ ID NO: 53. According to some embodiments, the vector comprises a nucleic acid sequence at least 85%, 90%, 95%, 96%, 97%, 98%, 99% identical to SEQ ID NO: 53. According to some embodiments, the vector comprises SEQ ID NO: 56. According to some embodiments, the vector comprises a nucleic acid sequence at least 85%, 90%, 95%, 96%, 97%, 98%, 99% identical to SEQ ID NO: 56. According to some embodiments, the vector comprises SEQ ID NO: 59. According to some embodiments, the vector comprises a nucleic acid sequence at least 85%, 90%, 95%, 96%, 97%, 98%, 99% identical to SEQ ID NO: 59. According to some embodiments, the vector comprises SEQ ID NO: 62. According to some embodiments, the vector comprises a nucleic acid sequence at least 85%, 90%, 95%, 96%, 97%, 98%, 99% identical to SEQ ID NO: 62. According to some embodiments, the vector comprises SEQ ID NO: 65. According to some embodiments, the vector comprises a nucleic acid sequence at least 85%, 90%, 95%, 96%, 97%, 98%, 99% identical to SEQ ID NO: 65. According to some embodiments, the vector comprises a nucleic acid sequence at least 85%, 90%, 95%, 96%, 97%, 98%, 99% identical to SEQ ID NO: 65. According to some embodiments, the vector comprises SEQ ID NO: 68. According to some embodiments, the vector comprises a nucleic acid sequence at least 85%, 90%, 95%, 96%, 97%, 98%, 99% identical to SEQ ID NO: 68. According to some embodiments, the vector comprises SEQ ID NO: 71. According to some embodiments, the vector comprises a nucleic acid sequence at least 85%, 90%, 95%, 96%, 97%, 98%, 99% identical to SEQ ID NO: 71.

According to other aspects, the disclosure provides a mammalian cell comprising the vector of any of the aspects and embodiments herein.

According to other aspects, the disclosure provides a method of making a recombinant adeno-associated viral (rAAV) vector comprising inserting into an adeno-associated viral vector a promoter; and at least one nucleic acid of any of the aspects and embodiments herein.

According to other aspects, the disclosure provides a method of making a recombinant adeno-associated viral (rAAV) vector comprising inserting into an adeno-associated viral vector; a promoter; at least one nucleic acid of any of the aspects and embodiments herein; a c9orf72 sense transcript specific inhibitor; and a c9orf72 antisense transcript specific inhibitor. According to some embodiments, the nucleic acid is a human nucleic acid. According to some embodiments, the serotype of the capsid sequence and the serotype of the ITRs of said AAV vector are independently selected from the group consisting of AAV1, AAV2, AAV3, AAV4, AAV5, AAV6, AAV7, AAV8, AAV9, AAV10, AAV11, and AAV12. According to some embodiments, the capsid sequence is a mutant capsid sequence.

According to other aspects, the disclosure provides a method of treating a c9orf72 associated disease, comprising administering to a subject in need thereof the vector of any of the aspects and embodiment herein, thereby treating the c9orf72 associated disease in the subject.

According to other aspects, the disclosure provides a method of preventing the progression of a c9orf72 associated disease, comprising administering to a subject in need thereof the vector of any of the aspects and embodiments herein, thereby treating the c9orf72 associated disease in the subject.

According to some embodiments, the c9orf72 associated disease is a c9orf72 hexanucleotide repeat expansion associated disease. According to some embodiments, the c9orf72 associated disease is a neurodegenerative disease. According to some embodiments, the neurodegenerative disease is selected from the group consisting of amyotrophic lateral sclerosis (ALS), frontotemporal dementia (FTD), Parkinson disease, progressive supranuclear palsy, ataxia, corticobasal syndrome, Huntington disease-like syndrome, Creutzfeldt-Jakob disease and Alzheimer disease. According to some embodiments, the neurodegenerative disease is amyotrophic lateral sclerosis (ALS) and/or frontotemporal dementia (FTD). According to some embodiments, the ALS is familial ALS or sporadic ALS. According to some embodiments, the subject has one or more mutations in the c9orf72 gene. According to some embodiments, the one or more mutations are selected from: one or more hexanucleotide repeat expansions, one or more nonsense mutations and one or more frame-shift mutations. According to some embodiments, the expression of c9orf72 is inhibited or suppressed. According to some embodiments, the c9orf72 is wild type c9orf72, mutated c9orf72 or both wild type c9orf72 and mutated c9orf72. According to some embodiments, the expression of c9orf72 is inhibited or suppressed by about 10% to about 100%, about 10% to about 90%, about 10% to about 70%, about 10% to about 50%, about 10% to about 30%, about 10% to about 20%, about 25% to about 75%, about 25% to about 50%, about 50% to about 75%, about 10%, about 20%, about 30%, about 40%, about 50%, about 60%, about 70%, about 80%, about 90% or more.

According to other aspects, the disclosure provides a method for inhibiting the expression of c9orf72 gene in a cell wherein the c9orf72 gene comprises a hexanucleotide repeat expansion, comprising administering the cell a composition comprising the vector of any of the aspects and embodiments herein. According to some embodiments, the hexanucleotide repeat expansion causes loss of function of c9orf72 protein and/or toxic gain of function from sense and antisense c9orf72 repeat RNA or from dipeptide repeats. According to some embodiments, the cell is a mammalian cell. According to some embodiments, the mammalian cell is a motor neuron or an astrocyte. According to some embodiments of any of the methods described herein, the vector is administered by intracranial administration. According to some embodiments, the intracranial administration comprises intrathecal or intracerebroventricular administration.

According to other aspects, the disclosure provides a kit comprising the vector of any of the aspects and embodiments herein, and instructions for use. According to some embodiments, the kit further comprises a device for intracranial administration delivery of the vector.

### BRIEF DESCRIPTION OF THE DRAWINGS

FIG. 1A is a schematic showing gene structure of c9orf72-AI. FIG. 1B shows the corresponding nucleic acid sequence (SEQ ID NO: 187).

FIG. 2 is a schematic showing gene supplementation of c9orf72.

FIG. 3A is a schematic showing the first open reading frame of an alternative translation of c9orf72. FIG. 3B shows the corresponding nucleic acid sequence (SEQ ID NO: 188). FIG. 3C is a schematic showing the second open reading frame after splicing of an alternative translation of c9orf72. FIG. 3D shows the corresponding nucleic acid sequence. (SEQ ID NO: 189).

FIG. 4 shows schematic constructs with selection marker.

FIG. 5 is a vector map of p084_EXPR_pcDNA_CBA_WTC9-EpiTag_WPRE.

FIG. 6 is a vector map of p085_EXPR_pcDNA_CASI_WTC9-EpiTag_WPRE.

FIG. 7 is a vector map of p111_EXPR-pcDNA-CBA-C9orf72-AI-loxp-WPRE-pA.

FIG. 8 is a vector map of p131_Expr_pcDNA-CBA-C9-mutAI-His-HA-WPRE-pA.

FIG. 9 is a vector map of p132_Expr_pcDNACBA-C9-AI-stop-His-HA-WPRE-pA.

FIG. 10 is a vector map of p133_Expr_pcDNA-CBA-C9-AI-Myc-Stop-His-HA-WPRE-pA.

FIG. 11 is a vector map of p134_Expr_pcDNA-CBA-C9-AI-Myc-stop-V2-His-Wpre_pA.

FIG. 12 is a graph showing high dynamic range generated by different promoters.

FIG. 13 shows schematic constructs and dose ranges.

FIG. 14 shows the results of the modulator test experiment.

FIG. 15 is a vector map of p141_EXPR_AAV_CBA-BFP_Antisense_miRNA1.

FIG. 16 is a vector map of p147_EXPR_AAV_CBA-BFP_sense_miRNA41.

FIG. 17 is a vector map of p136_Lenti_CBA_tandomarray-Sense-GA80s-GFP-WPRE.

FIG. 18 is a vector map of p137_Lenti_CBA_tandomarray-AntiSense-GA80s-GFP-WPRE.

FIG. 19 is a vector map of p138_Lenti_CBA_flex-Chronos-GA80s-GFP-WPRE.

FIG. 20 shows the results of miRNA knockdown experiment.

FIG. 21 shows a Western blot demonstrating expression of short isoform of C9orf72 protein.

### DETAILED DESCRIPTION

### I. Definitions

This disclosure is not limited to the particular methodology, protocols, cell lines, vectors, or reagents described herein because they may vary. Further, the terminology used herein is for the purpose of describing particular embodiments only and is not intended to limit the scope of the present disclosure.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “AAV” refers to adeno-associated virus, and may be used to refer to the recombinant virus vector itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on its serology, e.g., there are eleven serotypes of AAVs, AAV1-AAV11, and the term encompasses pseudotypes with the same properties.

As used herein, an “AAV vector” is meant to refer to a viral particle composed of at least one AAV capsid protein and an encapsidated polynucleotide. If the particle comprises a heterologous polynucleotide (i.e., a polynucleotide other than a wild-type AAV genome such as a transgene to be delivered to a mammalian cell), it can be referred to as “rAAV (recombinant AAV).” Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle).” An AAV “capsid protein” includes a capsid protein of a wild-type AAV, as well as modified forms of an AAV capsid protein which are structurally and or functionally capable of packaging an AAV genome and bind to at least one specific cellular receptor which may be different than a receptor employed by wild type AAV. A modified AAV capsid protein includes a chimeric AAV capsid protein such as one having amino acid sequences from two or more serotypes of AAV, e.g., a capsid protein formed from a portion of the capsid protein from AAV5 fused or linked to a portion of the capsid protein from AAV2, and a AAV capsid protein having a tag or other detectable non-AAV capsid peptide or protein fused or linked to the AAV capsid protein, e.g., a portion of an antibody molecule which binds the transferrin receptor may be recombinantly fused to the AAV-2 capsid protein.

As used herein, a “rAAV virus” or “rAAV viral particle” refers to a viral particle composed of at least one AAV capsid protein and an encapsidated rAAV vector genome.

As used herein, the terms “administer,” “administering,” “administration,” and the like, are meant to refer to methods that are used to enable delivery of therapeutics or pharmaceutical compositions to the desired site of biological action. According to certain embodiments, these methods include subretinal or intravitreal injection to an eye.

As used herein, “antisense activity” is meant to refer to any detectable or measurable activity attributable to the hybridization of an antisense compound to its target nucleic acid. In certain embodiments, antisense activity is a decrease in the amount or expression of a target nucleic acid or protein product encoded by such target nucleic acid.

As used herein, “antisense compound” is meant to refer to an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

As used herein, “antisense inhibition” is meant to refer to reduction of target nucleic acid levels in the presence of an antisense compound complementary to a target nucleic acid compared to target nucleic acid levels or in the absence of the antisense compound.

As used herein, “antisense oligonucleotide” is meant to refer to a single-stranded oligonucleotide having a nucleobase sequence that permits hybridization to a corresponding segment of a target nucleic acid. According to some embodiments, the antisense oligonucleotides of the present disclosure comprise at least 80%, at least about 85%, at least about 90%, at least about 95% sequence complementarity to a target region within the target nucleic acid. For example, an antisense compound in which 18 of 20 nucleobases of the antisense oligonucleotide are complementary, and would therefore specifically hybridize, to a target region would represent 90 percent complementarity. Percent complementarity of an antisense compound with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Antisense and other compounds of the disclosure, which hybridize to ABCD1 mRNA, are identified through experimentation, and representative sequences of these compounds are herein below identified as preferred embodiments of the disclosure.

As used herein, “c9orf72 antisense transcript” means transcripts produced from the non-coding strand (also called antisense strand and template strand) of the c9orf72 gene. The c9orf72 antisense transcript differs from the canonically transcribed “c9orf72 sense transcript”, which is produced from the coding strand (also called sense strand) of the c9orf72 gene.

As used herein, “c9orf72 associated disease” is meant to refer to means any disease associated with any c9orf72 nucleic acid or expression product thereof, regardless of which DNA strand the c9orf72 nucleic acid or expression product thereof is derived from. Such diseases may include a neurodegenerative disease. Such neurodegenerative diseases may include ALS and FTD.

As used herein, “c9orf72 hexanucleotide repeat expansion associated disease” means any disease associated with a c9orf72 nucleic acid containing a hexanucleotide repeat expansion. In certain embodiments, the hexanucleotide repeat expansion may comprise any of the following hexanucleotide repeats: GGGGCC, GGGGGG, GGGGGC, GGGGCG, GGCCCC, CCCCCC, GCCCCC, and/or CGCCCC. In certain embodiments, the hexanucleotide repeat is repeated at least 24 times. Such diseases may include a neurodegenerative disease. Such neurodegenerative diseases may include ALS and FTD.

As used herein, “c9orf72 nucleic acid” is meant to refer to any nucleic acid derived from the c9orf72 locus, regardless of which DNA strand the c9orf72 nucleic acid is derived from. In certain embodiments, a c9orf72 nucleic acid includes a DNA sequence encoding c9orf72, an RNA sequence transcribed from DNA encoding c9orf72 including genomic DNA comprising introns and exons (i.e., pre-mRNA), and an mRNA sequence encoding c9orf72. “c9orf72 mRNA” means an mRNA encoding a c9orf72 protein. In certain embodiments, a c9orf72 nucleic acid includes transcripts produced from the coding strand of the C9ORF72 gene. C9ORF72 sense transcripts are examples of c9orf72 nucleic acids. In certain embodiments, a c9orf72 nucleic acid includes transcripts produced from the non-coding strand of the c9orf72 gene. c9orf72 antisense transcripts are examples of c9orf72 nucleic acids.

As used herein, “c9orf72 transcript” is meant to refer to an RNA transcribed from c9orf72. In certain embodiments, a c9orf72 transcript is a c9orf72 sense transcript. In certain embodiments, a c9orf72 transcript is a c9orf72 antisense transcript.

As used herein, “cap structure” or “terminal cap moiety” is meant to refer to chemical modifications, which have been incorporated at either terminus of an antisense compound.

As used herein, “complementarity” is meant to refer to the capacity for pairing between nucleobases of a first nucleic acid and a second nucleic acid. “Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

As used herein, the term “carrier” is meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host. As used herein, the terms “expression vector,” “vector” or “plasmid” can include any type of genetic construct, including AAV or rAAV vectors, containing a nucleic acid or polynucleotide coding for a gene product in which part or all of the nucleic acid encoding sequence is capable of being transcribed and is adapted for gene therapy. The transcript can be translated into a protein. In some instances, it may be partially translated or not translated. In certain embodiments, expression includes both transcription of a gene and translation of mRNA into a gene product. In other embodiments, expression only includes transcription of the nucleic acid encoding genes of interest. An expression vector can also comprise control elements operatively linked to the encoding region to facilitate expression of the protein in target cells. The combination of control elements and a gene or genes to which they are operably linked for expression can sometimes be referred to as an “expression cassette.”

As used herein, the term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence.

As used herein, the term “gene delivery” means a process by which foreign DNA is transferred to host cells for applications of gene therapy.

As used herein, “gene supplementation” is meant to refer to replacing, altering, or supplementing a gene that is absent or abnormal and whose absence or abnormality is responsible for the disease. According to some embodiments, the c9orf72 gene is supplemented. According to some embodiments, the c9orf72 gene is mutated. According to some embodiments, the c9orf72 gene comprises one or more nonsense mutations. According to some embodiments, the c9orf72 gene comprises one or more frame-shift mutations.

As used herein, the term “heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, “hexanucleotide repeat expansion” is meant to refer to a series of six bases (for example, GGGGCC, GGGGGG, GGGGGC, GGGGCG, GGCCCC, CCCCCC, GCCCCC, and/or CGCCCC) repeated at least twice. In certain embodiments, the hexanucleotide repeat may be transcribed in the antisense direction from the c9orf72 gene. In certain embodiments, a pathogenic hexanucleotide repeat expansion includes at least 24 repeats of GGGGCC, GGGGGG, GGGGGC, GGGGCG, GGCCCC, CCCCCC, GCCCCC, and/or CGCCCC in a c9orf72 nucleic acid and is associated with disease. In certain embodiments, the repeats are consecutive. In certain embodiments, the repeats are interrupted by 1 or more nucleobases. In certain embodiments, a wild-type hexanucleotide repeat expansion includes 23 or fewer repeats of GGGGCC, GGGGGG, GGGGGC, GGGGCG, GGCCCC, CCCCCC, GCCCCC, and/or CGCCCC in a c9orf72 nucleic acid. In certain embodiments, the repeats are consecutive. In certain embodiments, the repeats are interrupted by 1 or more nucleobases.

As used herein, “hybridization” is meant to refer to the annealing of complementary nucleic acid molecules. In certain embodiments, complementary nucleic acid molecules include, but are not limited to, an antisense compound and a target nucleic acid. In certain embodiments, complementary nucleic acid molecules include, but are not limited to, an antisense oligonucleotide and a nucleic acid target.

As used herein, “inhibiting expression of a c9orf72 antisense transcript” is meant to refer to reducing the level or expression of a c9orf72 antisense transcript and/or its expression products (e.g., RAN translation products). In certain embodiments, c9orf72 antisense transcripts are inhibited in the presence of an antisense compound targeting a c9orf72 antisense transcript, including an antisense oligonucleotide targeting a c9orf72 antisense transcript, as compared to expression of c9orf72 antisense transcript levels in the absence of a C9ORF72 antisense compound, such as an antisense oligonucleotide.

As used herein, “inhibiting expression of a c9orf72 sense transcript” is meant to refer to reducing the level or expression of a c9orf72 sense transcript and/or its expression products (e.g., a c9orf72 mRNA and/or protein). In certain embodiments, c9orf72 sense transcripts are inhibited in the presence of an antisense compound targeting a c9orf72 sense transcript, including an antisense oligonucleotide targeting a c9orf72 sense transcript, as compared to expression of c9orf72 sense transcript levels in the absence of a c9orf72 antisense compound, such as an antisense oligonucleotide.

As used herein, “inverted terminal repeat” or “ITR” sequence is meant to refer to relatively short sequences found at the termini of viral genomes which are in opposite orientation. An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “wild-type ITR”, “WT-ITR” or “ITR” refers to the sequence of a naturally occurring ITR sequence in an AAV or other Dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

As used herein, an “isolated” molecule (e.g., nucleic acid or protein) or cell means it has been identified and separated and/or recovered from a component of its natural environment.

As used herein, “locked nucleic acid” or “LNA” or “LNA nucleosides” is meant to refer to nucleic acid monomers having a bridge connecting two carbon atoms between the 4′ and 2′ position of the nucleoside sugar unit, thereby forming a bicyclic sugar.

As used herein, the term “minimize”, “reduce”, “decrease,” and/or “inhibit” (and like terms) generally refers to the act of reducing, either directly or indirectly, a concentration, level, function, activity, or behavior relative to the natural, expected, or average, or relative to a control condition.

As used herein, “minimal regulatory element” is meant to refer to regulatory elements that are necessary for effective expression of a gene in a target cell and thus should be included in a transgene expression cassette. Such sequences could include, for example, promoter or enhancer sequences, a polylinker sequence facilitating the insertion of a DNA fragment within a plasmid vector, and sequences responsible for intron splicing and polyadenlyation of mRNA transcripts. In a recent example of a gene therapy treatment for achromatopsia, the expression cassette included the minimal regulatory elements of a polyadenylation site, splicing signal sequences, and AAV inverted terminal repeats. See, e.g., Komaromy et al.

As used herein, “mismatch” or “non-complementary nucleobase” is meant to refer to the case when a nucleobase of a first nucleic acid is not capable of pairing with the corresponding nucleobase of a second or target nucleic acid.

As used herein, “modified internucleoside linkage” is meant to refer to a substitution or any change from a naturally occurring internucleoside bond (i.e., a phosphodiester internucleoside bond).

As used herein, “modified nucleobase” is meant to refer to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

As used herein, “modified nucleoside” is meant to refer to nucleoside having, independently, a modified sugar moiety and/or modified nucleobase.

As used herein, “modified nucleotide” is meant to refer to a nucleotide having, independently, a modified sugar moiety, modified internucleoside linkage, and/or modified nucleobase.

As used herein, “modified oligonucleotide” is meant to refer to an oligonucleotide comprising at least one modified internucleoside linkage, modified sugar, and/or modified nucleobase.

As used herein, a “nucleic acid” is meant to refer to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

As used herein, “nucleobase” is meant to refer to heterocyclic moiety capable of pairing with a base of another nucleic acid.

As used herein, “nucleotide” is meant to refer to a nucleoside having a phosphate group covalently linked to the sugar portion of the nucleoside.

As used herein, “nucleoside” is meant to refer to a nucleobase linked to a sugar.

The asymmetric ends of DNA and RNA strands are called the 5′ (five prime) and 3′ (three prime) ends, with the 5′ end having a terminal phosphate group and the 3′ end a terminal hydroxyl group. The five prime (5′) end has the fifth carbon in the sugar-ring of the deoxyribose or ribose at its terminus. Nucleic acids are synthesized in vivo in the 5′- to 3′-direction, because the polymerase used to assemble new strands attaches each new nucleotide to the 3′-hydroxyl (—OH) group via a phosphodiester bond.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

A DNA sequence that “encodes” a particular PGRN protein (including fragments and portions thereof) is a nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “nRNA”).

As used herein, the terms “operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. An example of an alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

As used herein, “pharmaceutical composition” or “composition” is meant to refer to a composition or agent described herein (e.g. a recombinant adeno-associated (rAAV) expression vector), optionally mixed with at least one pharmaceutically acceptable chemical component, such as, though not limited to carriers, stabilizers, diluents, dispersing agents, suspending agents, thickening agents, excipients and the like.

As used herein, “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, a “promoter” is meant to refer to a region of DNA that facilitates the transcription of a particular gene. As part of the process of transcription, the enzyme that synthesizes RNA, known as RNA polymerase, attaches to the DNA near a gene. Promoters contain specific DNA sequences and response elements that provide an initial binding site for RNA polymerase and for transcription factors that recruit RNA polymerase.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate.

As used herein, “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, “region” is meant to refer to a portion of the target nucleic acid having at least one identifiable structure, function, or characteristic.

As used herein, “ribonucleotide” is meant to refer to a nucleotide having a hydroxy at the 2′ position of the sugar portion of the nucleotide. Ribonucleotides may be modified with any of a variety of substituents.

As used herein, “single-stranded oligonucleotide” is meant to refer to an oligonucleotide which is not hybridized to a complementary strand.

As used herein, “specifically hybridizable” is meant to refer to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

As used herein, “stringent hybridization conditions” or “stringent conditions” is meant to refer to conditions under which an oligomeric compound will hybridize to its target sequence, but to a minimal number of other sequences.

As used herein, a “subject” or “patient” or “individual” to be treated by the method of the invention is meant to refer to either a human or non-human animal. A “nonhuman animal” includes any vertebrate or invertebrate organism. A human subject can be of any age, gender, race or ethnic group, e.g., Caucasian (white), Asian, African, black, African American, African European, Hispanic, Middle eastern, etc. In some embodiments, the subject can be a patient or other subject in a clinical setting. In some embodiments, the subject is already undergoing treatment. In some embodiments, the subject is a neonate, infant, child, adolescent, or adult.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

As used herein, “targeting” or “targeted” is meant to refer to the process of design and selection of an antisense compound that will specifically hybridize to a target nucleic acid and induce a desired effect.

As used herein, “target nucleic acid,” “target RNA,” and “target RNA transcript” are meant to refer to a nucleic acid capable of being targeted by antisense compounds.

As used herein a “target region” is meant to refer to a portion of a target nucleic acid to which one or more antisense compounds is targeted.

As used herein, a “target segment” is meant to refer to the sequence of nucleotides of a target nucleic acid to which an antisense compound is targeted. “5′ target site” is meant to refer to the 5′-most nucleotide of a target segment. “3′ target site” is meant to refer to the 3′-most nucleotide of a target segment.

As used herein, “transgene” is meant to refer to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

A “transgene expression cassette” or “expression cassette” comprises the gene sequences that a nucleic acid vector is to deliver to target cells. These sequences include the gene of interest (e.g., CHF nucleic acids or variants thereof), one or more promoters, and minimal regulatory elements.

As used herein, “treatment” or “treating” a disease or disorder (such as, for example, a c9orf72 associated disease or a c9orf72 hexanucleotide repeat expansion associated disease, e.g. a neurodegenerative diseases, such as ALS or FTD) is meant to refer to alleviation of one or more signs or symptoms of the disease or disorder, diminishment of extent of disease or disorder, stabilized (e.g., not worsening) state of disease or disorder, preventing spread of disease or disorder, delay or slowing of disease or disorder progression, amelioration or palliation of the disease or disorder state, and remission (whether partial or total), whether detectable or undetectable. “Treatment” can also refer to prolonging survival as compared to expected survival if not receiving treatment.

As used herein, the phrase “unmodified nucleobases” refers to the purine bases adenine (A) and guanine (G), and the pyrimidine bases (T), cytosine (C), and uracil (U).

As used herein, the term “vector” refers to a recombinant plasmid or virus that comprises a nucleic acid to be delivered into a host cell, either in vitro or in vivo.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, a “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

As used herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

Transcriptional regulators refer to transcriptional activators and repressors that either activate or repress transcription of a gene of interest, such as c9orf72. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “including” is used herein to mean, and is used interchangeably with, the phrase “including but not limited to.”

The term “such as” is used herein to mean, and is used interchangeably, with the phrase “such as but not limited to.”

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims. Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### II. Nucleic Acids

The characterization and development of nucleic acid molecules for potential therapeutic use are provided herein. The present disclosure provides promoters, expression cassettes, vectors, kits, and methods that can be used in the treatment of a subject with a c9orf72 associated disease or a c9orf72 hexanucleotide repeat expansion associated disease (e.g., a neurodegenerative disease such as AML or FTD). In certain embodiments, the individual is at risk for developing a c9orf72 associated disease (e.g., a neurodegenerative disease, such as AML or FTD). Certain aspects of the disclosure relate to delivering a rAAV vector comprising a heterologous nucleic acid to cells which are relevant to the disease to be treated, e.g., in ALS the target cells are neurons, in particular embodiments motor neurons, and astrocytes.

According to some embodiments, the expressed c9orf72 protein is functional for the treatment of treatment of a c9orf72 associated disease or a c9orf72 hexanucleotide repeat expansion associated disease (e.g., a neurodegenerative disease such as AML or FTD). In some embodiments, the expressed c9orf72 protein does not cause an immune system reaction.

### Gene Supplementation

According to some aspects, the disclosure provides methods of treating a c9orf72 associated disease or a c9orf72 hexanucleotide repeat expansion associated disease (e.g., a neurodegenerative disease such as AML or FTD) by replacing, altering, or supplementing a c9orf72 gene that is absent or abnormal, and whose absence or abnormality is responsible for the disease. According to some embodiments, the c9orf72 gene comprises one or more nonsense mutations. According to some embodiments, the c9orf72 gene comprises one or more frame-shift mutations. According to some aspects, the disclosure provides methods of treating a c9orf72 associated disease or a c9orf72 hexanucleotide repeat expansion associated disease (e.g., a neurodegenerative disease such as AML or FTD) comprising delivery of a composition comprising rAAV vectors described herein to the subject, wherein the rAAV vector comprises a heterologous nucleic acid (e.g. a nucleic acid encoding c9orf72) and further comprising at least one AAV terminal repeat. According to some embodiments, the heterologous nucleic acid is operably linked to a promoter. According to some embodiments, the promoter is a neuron specific promoter, for example a human Synapsin 1 (hSyn) promoter. The hSyn promoter is particularly suited to use in the rAAVs described herein, due to its small size.

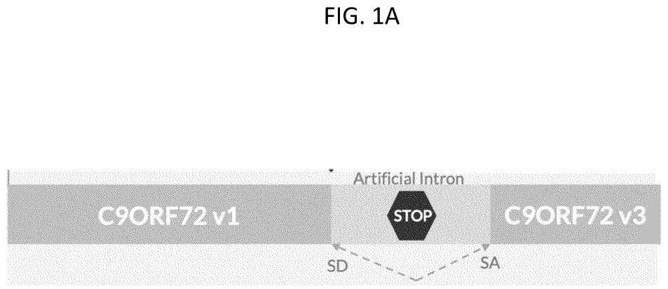

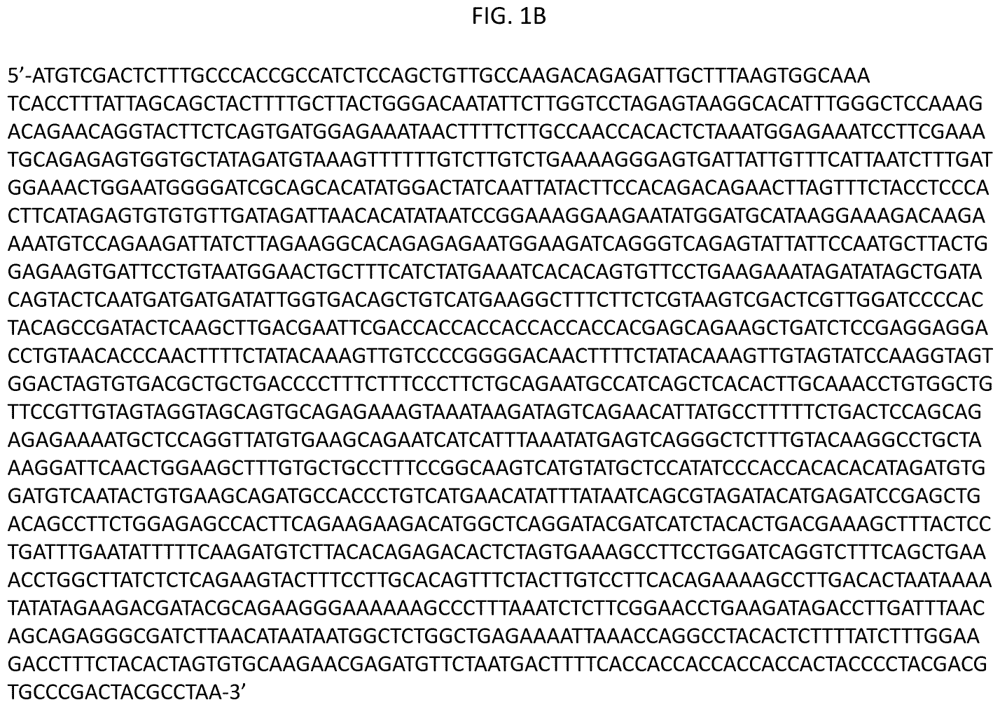

Two major mature mRNA transcript c9orf72 isoforms are expressed, v1 & v2, with proposed distinct intracellular functions: v1) regulates stress granule assembly in response to cellular stress; v2) does not seem to participate in stress granule assembly or regulation (Maharjan N. et al. 2017. Mol. Neurobiol. 54:3062-3077). The gene structure of c9orf72 is shown in FIG. 1.

Nucleotide sequences that encode c9orf72 include, but are not limited to, the following: the complement of GENBANK Accession No. NM_001256054.1 (SEQ ID NO: 53), GENBANK Accession No. NT_008413.18 truncated from nucleobase 27535000 to 27565000 (SEQ ID NO: 54) and the complement thereof (SEQ ID NO: 55), GENBANK Accession No. BQ068108.1 (incorporated herein as SEQ ID NO: 56), GENBANK Accession No. NM_018325.3 (incorporated herein as SEQ ID NO: 57), GENBANK Accession No. DN993522.1 (incorporated herein as SEQ ID NO: 58), GENBANK Accession No. NM_145005.5 (incorporated herein as SEQ ID NO: 59), GENBANK Accession No. DB079375.1 (incorporated herein as SEQ ID NO: 60), and GENBANK Accession No. BU194591.1 (incorporated herein as SEQ ID NO: 61).

According to some embodiments, the sequences described herein can further comprise one or more modifications to a sugar moiety, an internucleoside linkage, or a nucleobase.

According to certain embodiments, the nucleic acid is a human nucleic acid (i.e., a nucleic acid that is derived from a human c9Orf72 gene). In other embodiments, the nucleic acid is a non-human nucleic acid (i.e., a nucleic acid that is derived from a non-human c9Orf72 gene).

According to some embodiments, the AAV vectors comprise at least one nucleic acid region comprising one or more insertions, deletions, inversions, and/or substitutions. According to some embodiments, the AAV vectors described herein comprise at least one nucleic acid region which has been codon optimized. According to one embodiment, the nucleic acid encoding c9orf72 is codon optimized. According to one embodiment, the nucleic acid encoding c9orf72 is codon optimized for expression in a eukaryote, e.g., humans. According to some embodiments, a coding sequence encoding c9orf72 is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available.

A nucleic acid molecule (including, for example, a c9orf72 nucleic acid) of the present disclosure can be isolated using standard molecular biology techniques. Using all or a portion of a nucleic acid sequence of interest as a hybridization probe, nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

A nucleic acid molecule for use in the methods of the disclosure can also be isolated by the polymerase chain reaction (PCR) using synthetic oligonucleotide primers designed based upon the sequence of a nucleic acid molecule of interest. A nucleic acid molecule used in the methods of the disclosure can be amplified using cDNA, mRNA or, alternatively, genomic DNA as a template and appropriate oligonucleotide primers according to standard PCR amplification techniques.

Furthermore, oligonucleotides corresponding to nucleotide sequences of interest can also be chemically synthesized using standard techniques. Numerous methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein). Automated methods for designing synthetic oligonucleotides are available. See e.g., Hoover, D. M. & Lubowski, J. Nucleic Acids Research, 30(10): e43 (2002).

Many embodiments of the disclosure involve a c9orf72 nucleic acid Some aspects and embodiments of the disclosure involve other nucleic acids, such as isolated promoters or regulatory elements. A nucleic acid may be, for example, a cDNA or a chemically synthesized nucleic acid. A cDNA can be obtained, for example, by amplification using the polymerase chain reaction (PCR) or by screening an appropriate cDNA library. Alternatively, a nucleic acid may be chemically synthesized.

### Antisense Oligonucleotides

According to some embodiments, the disclosure provides antisense compounds. An antisense compound is capable of undergoing hybridization to a target nucleic acid through hydrogen bonding. According to certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted. Examples of antisense compounds include single-stranded and double-stranded compounds, such as, antisense oligonucleotides, siRNAs, shRNAs, ssRNAs, and occupancy-based compounds.

According to some embodiments, an antisense compound is targeted to a c9orf72 nucleic acid. According to some embodiments, an antisense compound that is targeted to a c9orf72 nucleic acid is 12 to 30 subunits in length. In other words, such antisense compounds are from 12 to 30 linked subunits. According to some embodiments, the antisense compound is 8 to 80, 12 to 50, 15 to 30, 18 to 24, 19 to 22, or 20 linked subunits. According to some embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. According to some embodiments the antisense compound is an antisense oligonucleotide, and the linked subunits are nucleosides.

According to some embodiments, the antisense compound is an shRNA that is targeted to a c9orf72 nucleic acid. Exemplary shRNAs are set forth in Table 1, below:

TABLE 1

| SEQ ID | |
|---|---|
| NO: | Sequence (5′-3′) |
| 1 | AGACATGATTACATTAATTAA |
| 2 | CCTCCTGTTTCTGAATACAAA |
| 3 | TCCTGGGAACTATCTAATTAA |
| 4 | AGTGAAAATTCTACAATCATA |
| 5 | TGATATTCACAGATTATGTTA |
| 6 | CCCTCCTGTTTCTGAATACAA |
| 7 | CAGACATGATTACATTAATTA |
| 8 | TCCCTGATTGGTATTTAGAAA |
| 9 | GATATTCACAGATTATGTTAA |
| 10 | GACAGTGAACTGTTTACAGTA |
| 11 | GGGAACTATCTAATTAACGTA |
| 12 | TGGCAACTGTTTGAATAGAAA |
| 13 | AACTGTTTGAATAGAAATTTA |
| 14 | CCCGGCTAAGTTTTTAATTTT |
| 15 | CCATACATGCAGACATGATTA |
| 16 | CCAAACAAAATATTTTATCAA |
| 17 | ACCGTATTTCAAGTATTCTGA |
| 18 | TCTGAGAAAAATCATATCTTA |
| 19 | CACAGATTATGTTAAAAGTTT |
| 20 | CCACTGCTATTGTAGTGAAAA |

According to some embodiments, the shRNA sequence comprises SEQ ID NO: 1. According to some embodiments, the shRNA sequence is 85% identical to SEQ ID NO: 1. According to some embodiments, the shRNA sequence is 90% identical to SEQ ID NO: 1. According to some embodiments, the shRNA sequence is 95%, 96%, 97% or 98% identical to SEQ ID NO: 1. According to some embodiments, the shRNA sequence is 99% identical to SEQ ID NO: 1. According to some embodiments, the shRNA sequence comprises SEQ ID NO: 2. According to some embodiments, the shRNA sequence is 85% identical to SEQ ID NO: 2. According to some embodiments, the shRNA sequence is 90% identical to SEQ ID NO: 2. According to some embodiments, the shRNA sequence is 95%, 96%, 97% or 98% identical to SEQ ID NO: 2. According to some embodiments, the shRNA sequence is 99% identical to SEQ ID NO: 2. According to some embodiments, the shRNA sequence comprises SEQ ID NO: 3. According to some embodiments, the shRNA sequence is 85% identical to SEQ ID NO: 3. According to some embodiments, the shRNA sequence is 90% identical to SEQ ID NO: 3. According to some embodiments, the shRNA sequence is 95%, 96%, 97% or 98% identical to SEQ ID NO: 3. According to some embodiments, the shRNA sequence is 99% identical to SEQ ID NO: 3. According to some embodiments, the shRNA sequence comprises SEQ ID NO: 4. According to some embodiments, the shRNA sequence is 85% identical to SEQ ID NO: 4. According to some embodiments, the shRNA sequence is 90% identical to SEQ ID NO: 4. According to some embodiments, the shRNA sequence is 95%, 96%, 97% or 98% identical to SEQ ID NO: 4. According to some embodiments, the shRNA sequence is 99% identical to SEQ ID NO: 4. According to some embodiments, the shRNA sequence comprises SEQ ID NO: 5. According to some embodiments, the shRNA sequence is 85% identical to SEQ ID NO: 5. According to some embodiments, the shRNA sequence is 90% identical to SEQ ID NO: 5. According to some embodiments, the shRNA sequence is 95%, 96%, 97% or 98% identical to SEQ ID NO: 5. According to some embodiments, the shRNA sequence is 99% identical to SEQ ID NO: 5. According to some embodiments, the shRNA sequence comprises SEQ ID NO: 6. According to some embodiments, the shRNA sequence is 85% identical to SEQ ID NO: 6. According to some embodiments, the shRNA sequence is 90% identical to SEQ ID NO: 6. According to some embodiments, the shRNA sequence is 95%, 96%, 97% or 98% identical to SEQ ID NO: 6. According to some embodiments, the shRNA sequence is 99% identical to SEQ ID NO: 6. According to some embodiments, the shRNA sequence comprises SEQ ID NO: 7. According to some embodiments, the shRNA sequence is 85% identical to SEQ ID NO: 7. According to some embodiments, the shRNA sequence is 90% identical to SEQ ID NO: 7. According to some embodiments, the shRNA sequence is 95%, 96%, 97% or 98% identical to SEQ ID NO: 7. According to some embodiments, the shRNA sequence is 99% identical to SEQ ID NO: 7. According to some embodiments, the shRNA sequence comprises SEQ ID NO: 8. According to some embodiments, the shRNA sequence is 85% identical to SEQ ID NO: 8. According to some embodiments, the shRNA sequence is 90% identical to SEQ ID NO: 8. According to some embodiments, the shRNA sequence is 95%, 96%, 97% or 98% identical to SEQ ID NO: 8. According to some embodiments, the shRNA sequence is 99% identical to SEQ ID NO: 8. According to some embodiments, the shRNA sequence comprises SEQ ID NO: 9. According to some embodiments, the shRNA sequence is 85% identical to SEQ ID NO: 9. According to some embodiments, the shRNA sequence is 90% identical to SEQ ID NO: 9. According to some embodiments, the shRNA sequence is 95%, 96%, 97% or 98% identical to SEQ ID NO: 9. According to some embodiments, the shRNA sequence is 99% identical to SEQ ID NO: 9. According to some embodiments, the shRNA sequence comprises SEQ ID NO: 10. According to some embodiments, the shRNA sequence is 85% identical to SEQ ID NO: 10. According to some embodiments, the shRNA sequence is 90% identical to SEQ ID NO: 10. According to some embodiments, the shRNA sequence is 95%, 96%, 97% or 98% identical to SEQ ID NO: 10. According to some embodiments, the shRNA sequence is 99% identical to SEQ ID NO: 10. According to some embodiments, the shRNA sequence comprises SEQ ID NO: 11. According to some embodiments, the shRNA sequence is 85% identical to SEQ ID NO: 11. According to some embodiments, the shRNA sequence is 90% identical to SEQ ID NO: 11. According to some embodiments, the shRNA sequence is 95%, 96%, 97% or 98% identical to SEQ ID NO: 11. According to some embodiments, the shRNA sequence is 99% identical to SEQ ID NO: 11. According to some embodiments, the shRNA sequence comprises SEQ ID NO: 12. According to some embodiments, the shRNA sequence is 85% identical to SEQ ID NO: 12. According to some embodiments, the shRNA sequence is 90% identical to SEQ ID NO: 12. According to some embodiments, the shRNA sequence is 95%, 96%, 97% or 98% identical to SEQ ID NO: 12. According to some embodiments, the shRNA sequence is 99% identical to SEQ ID NO: 12. According to some embodiments, the shRNA sequence comprises SEQ ID NO: 13. According to some embodiments, the shRNA sequence is 85% identical to SEQ ID NO: 13. According to some embodiments, the shRNA sequence is 90% identical to SEQ ID NO: 13. According to some embodiments, the shRNA sequence is 95%, 96%, 97% or 98% identical to SEQ ID NO: 13. According to some embodiments, the shRNA sequence is 99% identical to SEQ ID NO: 13.

According to some embodiments antisense oligonucleotides targeted to a c9orf72 nucleic acid may be shortened or truncated. For example, a single subunit may be deleted from the 5′ end (5′ truncation), or alternatively from the 3′ end (3′ truncation). A shortened or truncated antisense compound targeted to a c9orf72 nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

According to some embodiments, when a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end. Nucleotide sequences that encode c9orf72 are described above.

According to some embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for c9orf72 can be obtained by accession number from sequence databases such as NCBI. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. According to some embodiments, the desired effect is a reduction in mRNA target nucleic acid levels. According to some embodiments, the desired effect is a reduction of levels of protein encoded by the target nucleic acid or a phenotypic change associated with the target nucleic acid.

A target region may contain one or more target segments. Multiple target segments within a target region may be overlapping. Alternatively, they may be non-overlapping. According to some embodiments, target segments within a target region are separated by no more than about 300 nucleotides. According to some embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. According to some embodiments, target segments within a target region are separated by no more than, or no more than about, 5 nucleotides on the target nucleic acid. According to some embodiments, target segments are contiguous. Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within a target region. According to some embodiments, reductions in c9orf72 mRNA levels are indicative of inhibition of c9orf72 expression. Reductions in levels of a c9orf72 protein are also indicative of inhibition of target mRNA expression. Reduction in the presence of expanded c9orf72 RNA foci are indicative of inhibition of c9orf72 expression. Further, phenotypic changes are indicative of inhibition of c9orf72 expression. For example, improved motor function and respiration may be indicative of inhibition of c9orf72 expression.

According to some embodiments, hybridization occurs between an antisense compound disclosed herein and a c9orf72 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized. Methods of determining whether a sequence is specifically hybridizable to a target nucleic acid are well known in the art. In certain embodiments, the antisense compounds provided herein are specifically hybridizable with a c9orf72 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a c9orf72 nucleic acid).

Non-complementary nucleobases between an antisense compound and a c9orf72 nucleic acid may be tolerated provided that the antisense compound remains able to specifically hybridize to a target nucleic acid. Further, an antisense compound may hybridize over one or more segments of a c9orf72 nucleic acid such that intervening or adjacent segments are not involved in the hybridization event (e.g., a loop structure, mismatch or hairpin structure).

According to some embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a c9orf72 nucleic acid, a target region, target segment, or specified portion thereof. Percent complementarity of an antisense compound with a target nucleic acid can be determined using routine methods. For example, an antisense compound in which 18 of 20 nucleobases of the antisense compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an antisense compound which is 18 nucleobases in length having 4 (four) non-complementary nucleobases which are flanked by two regions of complete complementarity with the target nucleic acid would have 77.8% overall complementarity with the target nucleic acid and would thus fall within the scope of the present disclosure. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

According to some embodiments, the antisense compounds provided herein, or specified portions thereof, are fully complementary (i.e., 100% complementary) to a target nucleic acid, or specified portion thereof. For example, in some embodiments, an antisense compound may be fully complementary to a c9orf72 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

According to some embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a c9orf72 nucleic acid, or specified portion thereof. According to some embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a c9orf72 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. According to some embodiments, the antisense compounds, are complementary to at least an 8 nucleobase portion of a target segment. According to some embodiments, the antisense compounds are complementary to at least a 9 nucleobase portion of a target segment. According to some embodiments, the antisense compounds are complementary to at least a 10 nucleobase portion of a target segment. According to some embodiments, the antisense compounds, are complementary to at least an 11 nucleobase portion of a target segment. According to some embodiments, the antisense compounds, are complementary to at least a 12 nucleobase portion of a target segment. According to some embodiments, the antisense compounds, are complementary to at least a 13 nucleobase portion of a target segment. According to some embodiments, the antisense compounds, are complementary to at least a 14 nucleobase portion of a target segment. According to some embodiments, the antisense compounds, are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

The antisense compounds provided herein may also have a defined percent identity to a particular nucleotide sequence set forth herein (e.g., SEQ ID NOs 1-13). As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

According to some embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein. According to some embodiments, a portion of the antisense compound is compared to an equal length portion of the target nucleic acid. According to some embodiments, an 8, 9, 10, 11, 12, 13, 14, 15, 16, 17, 18, 19, 20, 21, 22, 23, 24, or 25 nucleobase portion is compared to an equal length portion of the target nucleic acid. According to some embodiments, a portion of the antisense oligonucleotide is compared to an equal length portion of the target nucleic acid. According to some embodiments, an 8, 9, 10, 11, 12, 13, 14, 15, 16, 17, 18, 19, 20, 21, 22, 23, 24, or 25 nucleobase portion is compared to an equal length portion of the target nucleic acid.

Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity. Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

According to some embodiments, antisense compounds targeted to a c9orf72 nucleic acid comprise one or more modified internucleoside linkages. According to some embodiments, the modified internucleoside linkages are interspersed throughout the antisense compound. According to some embodiments, the modified internucleoside linkages are phosphorothioate linkages. According to some embodiments, each internucleoside linkage of an antisense compound is a phosphorothioate internucleoside linkage. According to some embodiments, the antisense compounds targeted to a C9ORF72 nucleic acid comprise at least one phosphodiester linkage and at least one phosphorothioate linkage.

Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. According to some embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Nucleic acid sequences described herein can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences described herein can be stabilized against nucleolytic degradation such as by the incorporation of a modification, e.g., a nucleotide modification. For example, according to some embodiments, nucleic acid sequences described herein include a phosphorothioate at least the first, second, or third internucleotide linkage at the 5′ or 3′ end of the nucleotide sequence. According to some embodiments, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). According to some embodiments, the nucleic acid sequence can include at least one 2′-O-methyl-modified nucleotide, and in some embodiments, all of the nucleotides include a 2′-O-methyl modification.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

### III. Promoters, Expression Cassettes and Vectors

The promoters, c9orf72 nucleic acids, inhibitory oligonucleotides (RNAi), regulatory elements, and expression cassettes, and vectors of the disclosure may be produced using methods known in the art. The methods described below are provided as non-limiting examples of such methods.

In another aspect, the present disclosure provides vector constructs comprising a nucleotide sequence encoding the antibodies of the present disclosure and a host cell comprising such a vector.

### Promoters

A person skilled in the art may recognize that a target cell may require a specific promoter including but not limited to a promoter that is species specific, inducible, tissue-specific, or cell cycle-specific Pan et al., Nat. Med. 3:1145-9 (1997); the contents of which are herein incorporated by reference in its entirety). In one embodiment, the promoter is a promoter deemed to be efficient to drive the expression of the polynucleotides described herein. Promoters for which promote expression in most tissues include, for example, but are not limited to, human elongation factor 1α-subunit (EF1α), immediate-early cytomegalovirus (CMV), the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the telomerase (hTERT) promoter, chicken I3-actin (CBA) and its derivative CAG, the 13 glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β.), the synapsin (Syn), the methyl-CpG binding protein 2 (MeCP2), CaMKII, mGluR2, NFL, NFH, nβ2, PPE, Enk and EAAT2 promoters.

According to some embodiments, the promoter is the chimeric CMV-chicken ß-actin promoter (CBA) promoter.

In some embodiments, the promoter is capable of expressing the heterologous nucleic acid in a neuronal cell. In some embodiments, the promoter is capable of expressing the heterologous nucleic acid in a motor neuron cell. In some embodiments, the promoter is capable of expressing the heterologous nucleic acid in astrocytes. According to some embodiments, the promoter is a human Synapsin 1 (hSyn) promoter that is specific for neuronal cells. According to some embodiments, the promoter is a glial fibrillary acidic protein (GFAP) or EAAT2 promoter, that are specific for astrocytes.

In one embodiment, the AAV vector genome may comprise a promoter such as, but not limited to, CMV or U6. As a non-limiting example, the promoter for the AAV comprising the nucleic acid sequence for the siRNA molecules of the present disclosure is a CMV promoter. As another non-limiting example, the promoter for the AAV comprising the nucleic acid sequence for the siRNA molecules of the present disclosure is a U6 promoter.

In one embodiment, the AAV vector has an engineered promoter.

In one embodiment, the AAV vector further comprises an enhancer element.

In one embodiment, the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety) such as an intron. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The promoter may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

### Expression Cassettes

According to another aspect, the present disclosure provides a transgene expression cassette comprises (a) a promoter; (b) a nucleic acid comprising a c9orf72 nucleic acid as described herein; and (c) minimal regulatory elements. According to another aspect, the present disclosure provides a transgene expression cassette comprises (a) a promoter; (b) a nucleic acid comprising one or more antisense compounds as described herein; and (c) minimal regulatory elements. According to another aspect, the present disclosure provides a transgene expression cassette comprises (a) a promoter; (b) a nucleic acid comprising a c9orf72 nucleic acid as described herein; (c) a nucleic acid comprising one or more antisense compounds as described herein; and (d) minimal regulatory elements. A promoter of the disclosure includes the promoters discussed supra. According to some embodiments, the promoter is hSyn.

“Minimal regulatory elements” are regulatory elements that are necessary for effective expression of a gene in a target cell. Such regulatory elements could include, for example, promoter or enhancer sequences, a polylinker sequence facilitating the insertion of a DNA fragment within a plasmid vector, and sequences responsible for intron splicing and polyadenylation of mRNA transcripts. The expression cassettes of the disclosure may also optionally include additional regulatory elements that are not necessary for effective incorporation of a gene into a target cell.

### Vectors

The present disclosure also provides vectors that include any one of the expression cassettes discussed in the preceding section. According to some embodiments, the vector is an oligonucleotide that comprises the sequences of the expression cassette.

According to some embodiments, the vector is a viral vector, such as a vector derived from an adeno-associated virus, an adenovirus, a retrovirus, a lentivirus, a vaccinia/poxvirus, or a herpesvirus (e.g., herpes simplex virus (HSV)). See e.g., Howarth. In the most preferred embodiments, the vector is an adeno-associated viral (AAV) vector.

Multiple serotypes of adeno-associated virus (AAV), including 12 human serotypes (AAV1, AAV2, AAV3, AAV4, AAV5, AAV6, AAV7, AAV8, AAV9, AAV10, AAV11, and AAV12) and more than 100 serotypes from nonhuman primates have now been identified. Howarth J L et al., Using viral vectors as gene transfer tools. Cell Biol Toxicol 26:1-10 (2010) (hereinafter Howarth et al.). In embodiments of the present disclosure wherein the vector is an AAV vector, the serotype of the inverted terminal repeats (ITRs) of the AAV vector may be selected from any known human or nonhuman AAV serotype. In preferred embodiments, the serotype of the AAV ITRs of the AAV vector is selected from the group consisting of AAV1, AAV2, AAV3, AAV4, AAV5, AAV6, AAV7, AAV8, AAV9, AAV10, AAV11, and AAV12. Moreover, in embodiments of the present disclosure wherein the vector is an AAV vector, the serotype of the capsid sequence of the AAV vector may be selected from any known human or animal AAV serotype. In some embodiments, the serotype of the capsid sequence of the AAV vector is selected from the group consisting of AAV1, AAV2, AAV3, AAV4, AAV5, AAV6, AAV7, AAV8, AAV9, AAV10, AAV11, and AAV12. In preferred embodiments, the serotype of the capsid sequence is AAV5. In some embodiments wherein the vector is an AAV vector, a pseudotyping approach is employed, wherein the genome of one ITR serotype is packaged into a different serotype capsid. See e.g., Zolutuhkin S. et al. Production and purification of serotype 1, 2, and 5 recombinant adeno-associated viral vectors. Methods 28(2): 158-67 (2002). In preferred embodiments, the serotype of the AAV ITRs of the AAV vector and the serotype of the capsid sequence of the AAV vector are independently selected from the group consisting of AAV1, AAV2, AAV3, AAV4, AAV5, AAV6, AAV7, AAV8, AAV9, AAV10, AAV11, and AAV12.

In some embodiments of the present disclosure wherein the vector is a rAAV vector, a mutant capsid sequence is employed. Mutant capsid sequences, as well as other techniques such as rational mutagenesis, engineering of targeting peptides, generation of chimeric particles, library and directed evolution approaches, and immune evasion modifications, may be employed in the present disclosure to optimize AAV vectors, for purposes such as achieving immune evasion and enhanced therapeutic output. See e.g., Mitchell A. M. et al. AAV's anatomy: Roadmap for optimizing vectors for translational success. Curr Gene Ther. 10(5): 319-340.

AAV vectors can mediate long term gene expression in cells (e.g. neuronal cells) and elicit minimal immune responses making these vectors an attractive choice for gene delivery.

The antisense compounds (e.g. antisense oligonucleotides, siRNA molecules, shRNA molecules) of the present disclosure may be introduced into cells using any of a variety of approaches such as, but not limited to, viral vectors (e.g., AAV vectors). These viral vectors are engineered and optimized to facilitate the entry of siRNA molecule into cells that are not readily amendable to transfection. Also, some synthetic viral vectors possess an ability to integrate the shRNA into the cell genome, thereby leading to stable siRNA expression and long-term knockdown of a target gene. In this manner, viral vectors are engineered as vehicles for specific delivery while lacking the deleterious replication and/or integration features found in wild-type virus.

According to some embodiments, the antisense compounds (e.g. antisense oligonucleotides, siRNA molecules, shRNA molecules) of the present disclosure are introduced into a cell by contacting the cell with a composition comprising a lipophilic carrier and a vector, e.g., an AAV vector, comprising a nucleic acid sequence encoding the antisense compounds (e.g. antisense oligonucleotides, siRNA molecules, shRNA molecules) of the present disclosure. According to some embodiments, the antisense compounds (e.g. antisense oligonucleotides, siRNA molecules, shRNA molecules) are introduced into a cell by transfecting or infecting the cell with a vector, e.g., an AAV vector, comprising nucleic acid sequences capable of producing the antisense compounds (e.g. antisense oligonucleotides, siRNA molecules, shRNA molecules) when transcribed in the cell. According to some embodiments, the antisense compounds (e.g. antisense oligonucleotides, siRNA molecules, shRNA molecules) are introduced into a cell by injecting into the cell a vector, e.g., an AAV vector, comprising a nucleic acid sequence capable of producing the antisense compounds (e.g. antisense oligonucleotides, siRNA molecules, shRNA molecules) when transcribed in the cell.

According to some embodiments, prior to transfection, a vector, e.g., an AAV vector, comprising a nucleic acid sequence encoding the antisense compounds (e.g. antisense oligonucleotides, siRNA molecules, shRNA molecules) of the present disclosure may be transfected into cells.

According to other embodiments, the vectors, e.g., AAV vectors, comprising a nucleic acid sequence encoding the antisense compounds (e.g. antisense oligonucleotides, siRNA molecules, shRNA molecules) of the present disclosure may be delivered into cells by electroporation (e.g. U.S. Patent Publication No. 20050014264; the content of which is herein incorporated by reference in its entirety).

Other methods for introducing vectors, e.g., AAV vectors, comprising the nucleic acid sequence for the siRNA molecules described herein may include photochemical internalization as described in U. S. Patent publication No. 20120264807; the content of which is herein incorporated by reference in its entirety.

According to some embodiments, the formulations described herein may contain at least one vector, e.g., AAV vectors, comprising the nucleic acid sequence encoding antisense compounds (e.g. antisense oligonucleotides, siRNA molecules, shRNA molecules) described herein. According to some embodiments, the antisense compounds (e.g. antisense oligonucleotides, siRNA molecules, shRNA molecules) may target the c9orf72 gene at one target site. According to some embodiments, the formulation comprises a plurality of vectors, e.g., AAV vectors, each vector comprising a nucleic acid sequence encoding antisense compounds (e.g. antisense oligonucleotides, siRNA molecules, shRNA molecules) targeting the c9orf72 gene at a different target site. The c9orf72 gene may be targeted at 2, 3, 4, 5 or more than 5 sites.

According to some embodiments, the vectors, e.g., AAV vectors, from any relevant species, such as, but not limited to, human, dog, mouse, rat or monkey may be introduced into cells.

According to some embodiments, the vectors, e.g., AAV vectors, may be introduced into cells which are relevant to the disease to be treated. As a non-limiting example, the disease is ALS and the target cells are motor neurons and astrocytes.

According to some embodiments, the vectors, e.g., AAV vectors, may be introduced into cells which have a high level of endogenous expression of the target sequence.

According to some embodiments, the vectors, e.g., AAV vectors, may be introduced into cells which have a low level of endogenous expression of the target sequence.

According to some embodiments, the cells may be those which have a high efficiency of AAV transduction.

### IV. Methods of Producing Viral Vectors

The present disclosure also provides methods of making a recombinant adeno-associated viral (rAAV) vectors comprising inserting into an adeno-associated viral vector any one of the nucleic acids described herein. According to some embodiments, the rAAV vector further comprises one or more AAV inverted terminal repeats (ITRs).

According to the methods of making an rAAV vector that are provided by the disclosure, the serotype of the capsid sequence and the serotype of the ITRs of said AAV vector are independently selected from the group consisting of AAV1, AAV2, AAV3, AAV4, AAV5, AAV6, AAV7, AAV8, AAV9, AAV10, AAV11, and AAV12. Thus, the disclosure encompasses vectors that use a pseudotyping approach, wherein the genome of one ITR serotype is packaged into a different serotype capsid. See e.g., Daya S. and Berns, K. I., Gene therapy using adeno-associated virus vectors. Clinical Microbiology Reviews, 21(4): 583-593 (2008) (hereinafter Daya et al.). Furthermore, in some embodiments, the capsid sequence is a mutant capsid sequence.

### AAV Vectors

AAV vectors are derived from adeno-associated virus, which has its name because it was originally described as a contaminant of adenovirus preparations. AAV vectors offer numerous well-known advantages over other types of vectors: wildtype strains infect humans and nonhuman primates without evidence of disease or adverse effects; the AAV capsid displays very low immunogenicity combined with high chemical and physical stability which permits rigorous methods of virus purification and concentration; AAV vector transduction leads to sustained transgene expression in post-mitotic, non-dividing cells and provides long-term gain of function; and the variety of AAV subtypes and variants offers the possibility to target selected tissues and cell types. Heilbronn R & Weger S, Viral Vectors for Gene Transfer: Current Status of Gene Therapeutics, in M. Schafer-Korting (ed.), Drug Delivery, Handbook of Experimental Pharmacology, 197: 143-170 (2010) (hereinafter Heilbronn). A major limitation of AAV vectors is that the AAV offers only a limited transgene capacity (<4.9 kb) for a conventional vector containing single-stranded DNA.

AAV is a non-enveloped, small, single-stranded DNA-containing virus encapsidated by an icosahedral, 20 nm diameter capsid. The human serotype AAV2 was used in a majority of early studies of AAV. Heilbronn. It contains a 4.7 kb linear, single-stranded DNA genome with two open reading frames rep and cap (“rep” for replication and “cap” for capsid). Rep codes for four overlapping nonstructural proteins: Rep78, Rep68, Rep52, and Rep40. Rep78 and Rep69 are required for most steps of the AAV life cycle, including the initiation of AAV DNA replication at the hairpin-structured inverted terminal repeats (ITRs), which is an essential step for AAV vector production. The cap gene codes for three capsid proteins, VP1, VP2, and VP3. Rep and cap are flanked by 145 bp ITRs. The ITRs contain the origins of DNA replication and the packaging signals, and they serve to mediate chromosomal integration. The ITRs are generally the only AAV elements maintained in AAV vector construction.

To achieve replication, AAVs must be coinfected into the target cell with a helper virus (Grieger J C & Samulski R J, 2005. Adv Biochem Engin/Biotechnol 99:119-145). Typically, helper viruses are either adenovirus (Ad) or herpes simplex virus (HSV). In the absence of a helper virus, AAV can establish a latent infection by integrating into a site on human chromosome 19. Ad or HSV infection of cells latently infected with AAV will rescue the integrated genome and begin a productive infection. The four Ad proteins required for helper function are E1A, E1B, E4, and E2A. In addition, synthesis of Ad virus-associated (VA) RNAs is required. Herpesviruses can also serve as helper viruses for productive AAV replication. Genes encoding the helicase-primase complex (ULS, UL8, and UL52) and the DNA-binding protein (UL29) have been found sufficient to mediate the HSV helper effect. In some embodiments of the present disclosure that employ rAAV vectors, the helper virus is an adenovirus. In other embodiments that employ rAAV vectors, the helper virus is HSV.

Making Recombinant AAV (rAAV) Vectors

The production, purification, and characterization of the rAAV vectors of the present disclosure may be carried out using any of the many methods known in the art. For reviews of laboratory-scale production methods, see, e.g., Clark R K, Recent advances in recombinant adeno-associated virus vector production. Kidney Int. 61s:9-15 (2002); Choi V W et al., Production of recombinant adeno-associated viral vectors for in vitro and in vivo use. Current Protocols in Molecular Biology 16.25.1-16.25.24 (2007) (hereinafter Choi et al.); Grieger J C & Samulski R J, Adeno-associated virus as a gene therapy vector: Vector development, production, and clinical applications. Adv Biochem Engin/Biotechnol 99:119-145 (2005) (hereinafter Grieger & Samulski); Heilbronn R & Weger S, Viral Vectors for Gene Transfer: Current Status of Gene Therapeutics, in M. Schafer-Korting (ed.), Drug Delivery, Handbook of Experimental Pharmacology, 197: 143-170 (2010) (hereinafter Heilbronn); Howarth J L et al., Using viral vectors as gene transfer tools. Cell Biol Toxicol 26:1-10 (2010) (hereinafter Howarth). The production methods described below are intended as non-limiting examples.

AAV vector production may be accomplished by co-transfection of packaging plasmids (Heilbronn et al.,). The cell line supplies the deleted AAV genes rep and cap and the required helper virus functions. The adenovirus helper genes, VA-RNA, E2A and E4 are transfected together with the AAV rep and cap genes, either on two separate plasmids or on a single helper construct. A recombinant AAV vector plasmid wherein the AAV capsid genes are replaced with a transgene expression cassette (comprising the gene of interest, e.g., a c9orf72, and/or comprising the antisense compound (e.g. siRNA, shRNA, antisense oligonucleotides)) bracketed by ITRs, is also transfected. These packaging plasmids are typically transfected into 293 cells, a human cell line that constitutively expresses the remaining required Ad helper genes, E1A and E1B. This leads to amplification and packaging of the AAV vector carrying the gene of interest.

Multiple serotypes of AAV, including 12 human serotypes and more than 100 serotypes from nonhuman primates have now been identified. Howarth et al. The AAV vectors of the present disclosure may comprise capsid sequences derived from AAVs of any known serotype. As used herein, a “known serotype” encompasses capsid mutants that can be produced using methods known in the art. Such methods, include, for example, genetic manipulation of the viral capsid sequence, domain swapping of exposed surfaces of the capsid regions of different serotypes, and generation of AAV chimeras using techniques such as marker rescue. See Bowles et al. Marker rescue of adeno-associated virus (AAV) capsid mutants: A novel approach for chimeric AAV production. Journal of Virology, 77(1): 423-432 (2003), as well as references cited therein. Moreover, the AAV vectors of the present disclosure may comprise ITRs derived from AAVs of any known serotype. Preferentially, the ITRs are derived from one of the human serotypes AAV1-AAV12. In some embodiments of the present disclosure, a pseudotyping approach is employed, wherein the genome of one ITR serotype is packaged into a different serotype capsid.

Preferentially, the capsid sequences employed in the present disclosure are derived from one of the human serotypes AAV1-AAV12. Recombinant AAV vectors containing an AAV5 serotype capsid sequence have been demonstrated to target retinal cells in vivo. See, for example, Komaromy et al. Therefore, in preferred embodiments of the present disclosure, the serotype of the capsid sequence of the AAV vector is AAV5. In other embodiments, the serotype of the capsid sequence of the AAV vector is AAV1, AAV2, AAV3, AAV4, AAV6, AAV7, AAV8, AAV9, AAV10, AAV11, or AAV12. Even when the serotype of the capsid sequence does not naturally target retinal cells, other methods of specific tissue targeting may be employed. See Howarth et al. For example, recombinant AAV vectors can be directly targeted by genetic manipulation of the viral capsid sequence, particularly in the looped out region of the AAV three-dimensional structure, or by domain swapping of exposed surfaces of the capsid regions of different serotypes, or by generation of AAV chimeras using techniques such as marker rescue. See Bowles et al. 2003. Journal of Virology, 77(1): 423-432, as well as references cited therein.

One possible protocol for the production, purification, and characterization of recombinant AAV (rAAV) vectors is provided in Choi et al. Generally, the following steps are involved: design a transgene expression cassette, design a capsid sequence for targeting a specific receptor, generate adenovirus-free rAAV vectors, purify and titer. These steps are summarized below and described in detail in Choi et al.

The transgene expression cassette may be a single-stranded AAV (ssAAV) vector or a “dimeric” or self-complementary AAV (scAAV) vector that is packaged as a pseudo-double-stranded transgene. Choi et al.; Heilbronn; Howarth. Using a traditional ssAAV vector generally results in a slow onset of gene expression (from days to weeks until a plateau of transgene expression is reached) due to the required conversion of single-stranded AAV DNA into double-stranded DNA. In contrast, scAAV vectors show an onset of gene expression within hours that plateaus within days after transduction of quiescent cells. Heilbronn. However, the packaging capacity of scAAV vectors is approximately half that of traditional ssAAV vectors. Choi et al. Alternatively, the transgene expression cassette may be split between two AAV vectors, which allows delivery of a longer construct. See e.g., Daya et al. A ssAAV vector can be constructed by digesting an appropriate plasmid (such as, for example, a plasmid containing the c9orf72 gene) with restriction endonucleases to remove the rep and cap fragments, and gel purifying the plasmid backbone containing the AAVwt-ITRs. Choi et al. Subsequently, the desired transgene expression cassette can be inserted between the appropriate restriction sites to construct the single-stranded rAAV vector plasmid. A scAAV vector can be constructed as described in Choi et al.

Then, a large-scale plasmid preparation (at least 1 mg) of the rAAV vector and the suitable AAV helper plasmid and pXX6 Ad helper plasmid can be purified by double CsCl gradient fractionation. Choi et al. A suitable AAV helper plasmid may be selected from the pXR series, pXR1-pXR5, which respectively permit cross-packaging of AAV2 ITR genomes into capsids of AAV serotypes 1 to 5. The appropriate capsid may be chosen based on the efficiency of the capsid's targeting of the cells of interest. Known methods of varying genome (i.e., transgene expression cassette) length and AAV capsids may be employed to improve expression and/or gene transfer to specific cell types (e.g., neuronal cells).

Next, 293 cells are transfected with pXX6 helper plasmid, rAAV vector plasmid, and AAV helper plasmid. Choi et al. Subsequently the fractionated cell lysates are subjected to a multistep process of rAAV purification, followed by either CsCl gradient purification or heparin sepharose column purification. The production and quantitation of rAAV virions may be determined using a dot-blot assay. In vitro transduction of rAAV in cell culture can be used to verify the infectivity of the virus and functionality of the expression cassette.

In addition to the methods described in Choi et al., various other transfection methods for production of AAV may be used in the context of the present disclosure. For example, transient transfection methods are available, including methods that rely on a calcium phosphate precipitation protocol.

In addition to the laboratory-scale methods for producing rAAV vectors, the present disclosure may utilize techniques known in the art for bioreactor-scale manufacturing of AAV vectors, including, for example, Heilbronn; Clement, N. et al. Large-scale adeno-associated viral vector production using a herpesvirus-based system enables manufacturing for clinical studies. Human Gene Therapy, 20: 796-606.

### V. Methods of Treatment

The present disclosure provides methods of gene therapy for c9orf72 associated diseases, for example neurodegenerative diseases, such as ALS and FTD. A hexanucleotide GGGGCC repeat expansion in the C9orf72 gene is the most frequent genetic cause of both ALS and FTD in Europe and North America. The vast majority (>95%) of neurologically healthy individuals have ≤11 hexanucleotide repeats in the C9orf72 gene (Rutherford et al., Neurobiol Aging. 2012 December; 33(12):2950.e5-7). The GGGGCC-expansion lies in the 5′ region of C9orf72 intron 1. The expanded GGGGCC repeats are bidirectionally transcribed into repetitive RNA, which forms sense and antisense RNA foci (Mizielinska et al. 2013. Acta Neuropathol. December; 126(6):845-57; Gendron et al. 2013. Acta Neuropathol. December; 126(6):829-44). Despite being within a non-coding region of C9orf72, these repetitive RNAs can be translated in every reading frame to form five different dipeptide repeat proteins (DPRs)—poly-GA, poly-GP poly-GR, poly-PA and poly-PR—via a non-canonical mechanism known as repeat-associated non-ATG (RAN) translation (Zu et al. 2013. Proc Natl Acad Sci USA. December 17; 110(51):E4968-77; Mori et al., Acta Neuropathol. 2013 December; 126(6):881-93). Three transcript variants (V1, V2, V3) have been described for the C9orf72 gene: V2 and V3 utilize exon 1a and therefore include the hexanucleotide repeat, while V1 utilizes the alternative exon 1b therefore excluding the hexanucleotide repeat, which is located upstream of the transcription start site.

Competing but non-exclusive mechanisms have arisen in understanding the pathogenenic effects of hexanucleotide repeats: loss of function of C9orf72 protein, and toxic gain of function from sense and antisense C9orf72 repeat RNA or from DPRs. C9orf72 repeat expansions have also been identified as a rare cause of other neurodegenerative diseases, including Parkinson disease, progressive supranuclear palsy, ataxia, corticobasal syndrome, Huntington disease-like syndrome, Creutzfeldt-Jakob disease and Alzheimer disease. According to some embodiments, the c9orf72 associated disease is a c9orf72 hexanucleotide repeat expansion associated disease.

Amyotrophic lateral sclerosis (ALS), an adult-onset neurodegenerative disorder, is a progressive and fatal disease characterized by the selective death of motor neurons in the motor cortex, brainstem and spinal cord. The incidence of ALS is about 1.9 per 100,000. Patients diagnosed with ALS develop a progressive muscle phenotype characterized by spasticity, hyperreflexia or hyporeflexia, fasciculations, muscle atrophy and paralysis. These motor impairments are caused by the denervation of muscles due to the loss of motor neurons. The major pathological features of ALS include degeneration of the corticospinal tracts and extensive loss of lower motor neurons (LMNs) or anterior horn cells (Ghatak et al. 1986. J Neuropathol Exp Neurol. 45, 385-395), degeneration and loss of Betz cells and other pyramidal cells in the primary motor cortex (Udaka et al. 1986. Acta Neuropathol. 70, 289-295; Maekawa et al., Brain, 2004, 127, 1237-1251) and reactive gliosis in the motor cortex and spinal cord (Kawamata et al., Am J Pathol., 1992, 140, 691-707; and Schiffer et al., J Neurol Sci., 1996, 139, 27-33). ALS is usually fatal within 3 to 5 years after the diagnosis due to respiratory defects and/or inflammation (Rowland L P and Shneibder N A, N Engl. J. Med., 2001, 344, 1688-1700).

A cellular hallmark of ALS is the presence of proteinaceous, ubiquitinated, cytoplasmic inclusions in degenerating motor neurons and surrounding cells (e.g., astrocytes). Ubiquitinated inclusions (i.e., Lewy body-like inclusions or Skein-like inclusions) are the most common and specific type of inclusion in ALS and are found in lower motor neurons (LMNs) of the spinal cord and brainstem, and in corticospinal upper motor neurons (UMNs) (Matsumoto et al., J Neurol Sci., 1993, 115, 208-213; and Sasak and Maruyama, Acta Neuropathol., 1994, 87, 578-585). A few proteins have been identified to be components of the inclusions, including ubiquitin, Cu/Zn superoxide dismutase 1 (SOD1), peripherin and dorfin. Neurofilamentous inclusions are often found in hyaline conglomerate inclusions (HCIs) and axonal ‘spheroids’ in spinal cord motor neurons in ALS. Other types and less specific inclusions include Bunina bodies (cystatin C-containing inclusions) and Crescent shaped inclusions (SCIs) in upper layers of the cortex. Other neuropathological features seen in ALS include fragmentation of the Golgi apparatus, mitochondrial vacuolization and ultrastructural abnormalities of synaptic terminals (Fujita et al., Acta Neuropathol. 2002, 103, 243-247).

In addition, in frontotemporal dementia ALS (FTD-ALS) cortical atrophy (including the frontal and temporal lobes) is also observed, which may cause cognitive impairment in FTD-ALS patients.

ALS is a complex and multifactorial disease and multiple mechanisms hypothesized as responsible for ALS pathogenesis include, but are not limited to, dysfunction of protein degradation, glutamate excitotoxicity, mitochondrial dysfunction, apoptosis, oxidative stress, inflammation, protein misfolding and aggregation, aberrant RNA metabolism, and altered gene expression.

About 10%-15% of ALS cases have family history of the disease, and these patients are referred to as familial ALS (fALS) or inherited patients, commonly with a Mendelian dominant mode of inheritance and high penetrance. The remainder (approximately 85%-95%) is classified as sporadic ALS (sALS), as they are not associated with a documented family history, but instead are thought to be due to other risk factors including, but not limited to environmental factors, genetic polymorphisms, somatic mutations, and possibly gene-environmental interactions. In most cases, familial (or inherited) ALS is inherited as autosomal dominant disease, but pedigrees with autosomal recessive and X-linked inheritance and incomplete penetrance exist. Sporadic and familial forms are clinically indistinguishable suggesting a common pathogenesis. The precise cause of the selective death of motor neurons in ALS remains elusive. Progress in understanding the genetic factors in familial ALS may shed light on both forms of the disease.

According to some embodiments, the present disclosure provides methods for treating a c9orf72 associated disease by administering to a subject in need thereof a therapeutically effective amount of a plasmid or AAV vector described herein. The ALS may be familial ALS or sporadic ALS. According to some embodiments, the c9orf72 associated disease is a c9orf72 hexanucleotide repeat expansion associated disease. According to some embodiments, the c9orf72 associated disease is ALS. According to some embodiments, the c9orf72 associated disease is FTD. According to some embodiments, the subject has one or more c9orf72 hexanucleotide repeat expansions. According to some embodiments, the subject has one or more c9orf72 nonsense mutations. According to some embodiments, the subject has one or more c9orf72 frame shift mutations.

According to some embodiments, the present disclosure provides methods for treating ALS by administering to a subject in need thereof a therapeutically effective amount of a plasmid or AAV vector described herein. The ALS may be familial ALS or sporadic ALS.

According to some embodiments, the present disclosure provides methods for treating FTD by administering to a subject in need thereof a therapeutically effective amount of a plasmid or AAV vector described herein.

According to some embodiments, the subject is identified by the following criteria: 1) clinical behavioral biomarkers reported from physicians; 2) signs of disease progression; 3) genome and/or transcriptome sequencing for c9orf72 locus.

In any of the methods of treatment, the vector can be any type of vector known in the art. According to some embodiments, the vector is a viral vector, such as a vector derived from an adeno-associated virus, an adenovirus, a retrovirus, a lentivirus, a vaccinia/poxvirus, or a herpesvirus (e.g., herpes simplex virus (HSV)). See e.g., Howarth. According to preferred embodiments, the vector is an adeno-associated viral (AAV) vector. Nucleic acid sequences described herein can be inserted into delivery vectors and expressed from transcription units within the vectors (e.g., AAV vectors). The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the disclosure into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the disclosure can be constructed based on viral backbones including, but not limited to, a retrovirus, lentivirus, adenovirus, adeno-associated virus, pox virus or alphavirus. The recombinant vectors capable of expressing the nucleic acids of the disclosure can be delivered as described herein, and persist in target cells (e.g., stable transformants).

According to some embodiments, the composition comprising the vectors, e.g., AAV vectors, comprising a nucleic acid sequence encoding the antisense compounds (e.g. antisense oligonucleotides, siRNA molecules, shRNA molecules) of the present disclosure is administered to the central nervous system of the subject. In other embodiments, the composition comprising the vectors, e.g., AAV vectors, comprising a nucleic acid sequence encoding the siRNA molecules of the present disclosure is administered to motor neurons. In other embodiments, the composition comprising the vectors, e.g., AAV vectors, comprising a nucleic acid sequence encoding the siRNA molecules of the present disclosure is administered to astrocytes.

According to some embodiments, the vectors, e.g., AAV vectors, comprising a nucleic acid sequence encoding the antisense compounds (e.g. antisense oligonucleotides, siRNA molecules, shRNA molecules) of the present disclosure may be delivered into specific types of targeted cells, including motor neurons; glial cells including oligodendrocyte, astrocyte and microglia; and/or other cells surrounding neurons such as T cells.

According to some embodiments, the vectors, e.g., AAV vectors, comprising a nucleic acid sequence encoding the antisense compounds (e.g. antisense oligonucleotides, siRNA molecules, shRNA molecules) of the present disclosure may be used as a therapy for ALS.

According to some embodiments, the present composition is administered as a solo therapeutics or combination therapeutics for the treatment of ALS.

The vectors, e.g., AAV vectors, encoding antisense compounds (e.g. antisense oligonucleotides, siRNA molecules, shRNA molecules) targeting the c9orf72 gene may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

According to some embodiments, therapeutic agents that may be used in combination with the vectors, e.g., AAV vectors, encoding the nucleic acid sequence for the antisense compounds (e.g. antisense oligonucleotides, siRNA molecules, shRNA molecules) of the present disclosure can be small molecule compounds which are antioxidants, anti-inflammatory agents, anti-apoptosis agents, calcium regulators, antiglutamatergic agents, structural protein inhibitors, and compounds involved in metal ion regulation.

According to some embodiments, compounds for treating ALS which may be used in combination with the vectors described herein include, but are not limited to, antiglutamatergic agents: Riluzole, Topiramate, Talampanel, Lamotrigine, Dextromethorphan, Gabapentin and AMPA antagonist; Anti-apoptosis agents: Minocycline, Sodium phenylbutyrate and Arimoclomol; Anti-inflammatory agent: ganglioside, Celecoxib, Cyclosporine, Azathioprine, Cyclophosphamide, Plasmaphoresis, Glatiramer acetate and thalidomide; Ceftriaxone (Berry et al., Plos One, 2013, 8(4)); Beat-lactam antibiotics; Pramipexole (a dopamine agonist) (Wang et al., Amyotrophic Lateral Scler., 2008, 9(1), 50-58); Nimesulide, described in U.S. Patent Publication No. 20060074991; Diazoxide, described in U.S. Patent Publication No. 20130143873); pyrazolone derivatives, described in US Patent Publication No. 20080161378; free radical scavengers that inhibit oxidative stress-induced cell death, such as bromocriptine (US. Patent Publication No. 20110105517); phenyl carbamate compounds discussed in PCT Patent Publication No. 2013100571; neuroprotective compounds, described in U.S. Pat. Nos. 6,933,310 and 8,399,514 and US Patent Publication Nos. 20110237907 and 20140038927; and glycopeptides, described in U.S. Patent Publication No. 20070185012; the content of each of which is incorporated herein by reference in their entirety.

According to some embodiments, therapeutic agents that may be used in combination therapy with the vectors, e.g., AAV vectors, encoding the nucleic acid sequence for the antisense compounds (e.g. antisense oligonucleotides, siRNA molecules, shRNA molecules) of the present disclosure may be hormones or variants that can protect neuronal loss, such as adrenocorticotropic hormone (ACTH) or fragments thereof (e.g., U.S. Patent Publication No. 20130259875); Estrogen (e.g., U.S. Pat. Nos. 6,334,998 and 6,592,845); the content of each of which is incorporated herein by reference in their entirety.

According to some embodiments, neurotrophic factors may be used in combination therapy with the vectors, e.g., AAV vectors, encoding the nucleic acid sequence for the siRNA molecules of the present disclosure for treating ALS. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

According to some embodiments, the composition of the present disclosure for treating ALS is administered to the subject in need intravenously, intramuscularly, subcutaneously, intraperitoneally, intrathecally and/or intraventricularly, allowing the siRNA molecules or vectors comprising the siRNA molecules to pass through one or both the blood-brain barrier and the blood spinal cord barrier. According to some embodiments, the method includes administering (e.g., intraventricularly administering and/or intrathecally administering) directly to the central nervous system (CNS) of a subject (using, e.g., an infusion pump and/or a delivery scaffold) a therapeutically effective amount of a composition comprising vectors, e.g., AAV vectors, encoding the nucleic acid sequence for the antisense compounds (e.g. antisense oligonucleotides, siRNA molecules, shRNA molecules) of the present disclosure. The vectors may be used to silence or suppress c9orf72 gene expression, and/or reducing one or more symptoms of ALS in the subject such that ALS is therapeutically treated.

According to some embodiments, the symptoms of ALS include, but are not limited to, motor neuron degeneration, muscle weakness, muscle atrophy, the stiffness of muscle, difficulty in breathing, slurred speech, fasciculation development, frontotemporal dementia and/or premature death are improved in the subject treated. In other aspects, the composition of the present disclosure is applied to one or both of the brain and the spinal cord. According to some embodiments, one or both of muscle coordination and muscle function are improved. According to some embodiments, the survival of the subject is prolonged.

According to some embodiments, administration of the vectors, e.g., AAV vectors encoding antisense compounds (e.g. antisense oligonucleotides, siRNA molecules, shRNA molecules) of the disclosure to a subject may lower mutant c9orf72 (e.g. c9orf72 comprising hexanucleotide repeat expansions) in the CNS of a subject. In another embodiment, administration of the vectors, e.g., AAV vectors, to a subject may lower wild-type c9orf72 in the CNS of a subject. In yet another embodiment, administration of the vectors, e.g., AAV vectors, to a subject may lower both mutant c9orf72 and wild-type c9orf72 in the CNS of a subject. The mutant and/or wild-type c9orf72 may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the CNS, a region of the CNS, or a specific cell of the CNS of a subject.

According to some embodiments, reduction of expression of the mutant and/or wild-type c9orf72 will reduce the effects of ALS in a subject.

According to some embodiments, the vectors, e.g., AAV vectors described herein, may be administered to a subject who is in the early stages of ALS. Early stage symptoms include, but are not limited to, muscles which are weak and soft or stiff, tight and spastic, cramping and twitching (fasciculations) of muscles, loss of muscle bulk (atrophy), fatigue, poor balance, slurred words, weak grip, and/or tripping when walking. The symptoms may be limited to a single body region or a mild symptom may affect more than one region. As a non-limiting example, administration of the vectors, e.g., AAV vectors described herein, may reduce the severity and/or occurrence of the symptoms of ALS.

According to some embodiments, the vectors, e.g., AAV vectors described herein, may be administered to a subject who is in the middle stages of ALS. The middle stage of ALS includes, but is not limited to, more widespread muscle symptoms as compared to the early stage, some muscles are paralyzed while others are weakened or unaffected, continued muscle twitchings (fasciculations), unused muscles may cause contractures where the joints become rigid, painful and sometimes deformed, weakness in swallowing muscles may cause choking and greater difficulty eating and managing saliva, weakness in breathing muscles can cause respiratory insufficiency which can be prominent when lying down, and/or a subject may have bouts of uncontrolled and inappropriate laughing or crying (pseudobulbar affect). As a non-limiting example, administration of the vectors, e.g., AAV vectors described herein, may reduce the severity and/or occurrence of the symptoms of ALS.

According to some embodiments, the vectors, e.g., AAV vectors described herein, may be administered to a subject who is in the late stages of ALS. The late stage of ALS includes, but is not limited to, voluntary muscles which are mostly paralyzed, the muscles that help move air in and out of the lungs are severely compromised, mobility is extremely limited, poor respiration may cause fatigue, fuzzy thinking, headaches and susceptibility to infection or diseases (e.g., pneumonia), speech is difficult and eating or drinking by mouth may not be possible.

According to some embodiments, the vectors, e.g., AAV vectors described herein, may be used to treat a subject with ALS who has a C9orf72 mutation.

According to some embodiments, the vectors, e.g., AAV vectors described herein, may be used to treat a subject with ALS who has TDP-43 mutations.

According to some embodiments, the vectors, e.g., AAV vectors described herein, may be used to treat a subject with ALS who has FUS mutations.

According to some embodiments, the nucleic acid sequences described herein are directly introduced into a cell, where the nucleic acid sequences are expressed to produce the encoded product, prior to administration in vivo of the resulting recombinant cell. This can be accomplished by any of numerous methods known in the art, e.g., by such methods as electroporation, lipofection, calcium phosphate mediated transfection.

### Pharmaceutical Compositions

According to some aspects, the disclosure provides pharmaceutical compositions comprising any of the vectors described herein, optionally in a pharmaceutically acceptable excipient.

In addition to the pharmaceutical compositions (vectors, e.g., AAV vectors comprising the nucleic acid sequence encoding the antisense compounds (e.g. antisense oligonucleotides, siRNA molecules, shRNA molecules), provided herein are pharmaceutical compositions which are suitable for administration to humans, it will be understood by the skilled artisan that such compositions are generally suitable for administration to any other animal, e.g., to non-human animals, e.g. non-human mammals Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

According to some embodiments, compositions are administered to humans, human patients or subjects. For the purposes of the present disclosure, the phrase “active ingredient” generally refers either to the synthetic siRNA duplexes, the vector, e.g., AAV vector, encoding the siRNA duplexes, or to the siRNA molecule delivered by a vector as described herein.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

The vectors e.g., AAV vectors, comprising the nucleic acid sequence encoding the antisense compounds (e.g. antisense oligonucleotides, siRNA molecules, shRNA molecules) of the present disclosure can be formulated using one or more excipients to: (1) increase stability; (2) increase cell transfection or transduction; (3) permit the sustained or delayed release; or (4) alter the biodistribution (e.g., target the viral vector to specific tissues or cell types such as brain and motor neurons).

According to some aspects, the disclosure provides pharmaceutical compositions comprising any of the antisense compounds described herein, optionally in a pharmaceutically acceptable excipient.

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to a c9orf72 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a C9ORF72 nucleic acid and a pharmaceutically acceptable diluent. According to some embodiments, the pharmaceutically acceptable diluent is PBS. According to some embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

Excipients, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21.sup.st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

According to some embodiments, the formulations may comprise at least one inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of vectors comprising the nucleic acid sequence for the antisense compounds (e.g. antisense oligonucleotides, siRNA molecules, shRNA molecules) molecules of the present disclosure may include cations or anions. According to some embodiments, the formulations include metal cations such as, but not limited to, Zn2+, Ca2+, Cu2+, Mg+ and combinations thereof.

As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977); the content of each of which is incorporated herein by reference in their entirety.

According to some embodiments, the vector, e.g., AAV vector, comprising the nucleic acid sequence for the antisense compounds (e.g. antisense oligonucleotides, siRNA molecules, shRNA molecules) of the present disclosure may be formulated for CNS delivery. Agents that cross the brain blood barrier may be used. For example, some cell penetrating peptides that can target siRNA molecules to the brain blood barrier endothelium may be used to formulate the siRNA duplexes targeting the SOD1 gene (e.g., Mathupala, Expert Opin Ther Pat., 2009, 19, 137-140; the content of which is incorporated herein by reference in its entirety)

### Administration and Dosing

According to the methods of treatment of the present disclosure, administering of a compositions comprising a vector described herein can be accomplished by any means known in the art. According to some embodiments, compositions of vector, e.g., AAV vector, comprising a nucleic acid sequence described herein (e.g. antisense compounds (e.g. antisense oligonucleotides, siRNA molecules, shRNA molecules)) may be administered in a way which facilitates the vectors or siRNA molecule to enter the central nervous system and penetrate into motor neurons.

According to some embodiments, the vector, e.g., an AAV vector, comprising a nucleic acid sequence encoding antisense compounds (e.g. antisense oligonucleotides, siRNA molecules, shRNA molecules) of the present disclosure may be administered by muscular injection.

According to some embodiments, AAV vectors that express antisense compounds (e.g. antisense oligonucleotides, siRNA molecules, shRNA molecules) of the present disclosure may be administered to a subject by peripheral injections and/or intranasal delivery. It was disclosed in the art that the peripheral administration of AAV vectors for siRNA duplexes can be transported to the central nervous system, for example, to the motor neurons (e.g., U.S. Patent Publication Nos. 20100240739; and 20100130594; the content of each of which is incorporated herein by reference in their entirety).

According to some embodiments, compositions comprising at least one vector, e.g., an AAV vector, comprising a nucleic acid sequence encoding the antisense compounds (e.g. antisense oligonucleotides, siRNA molecules, shRNA molecules) of the present disclosure may be administered to a subject by intracranial delivery (e.g. intrathecal or intracerebroventricular administration, see e.g., U.S. Pat. No. 8,119,611; the content of which is incorporated herein by reference in its entirety).

The vector, e.g., an AAV vector, comprising a nucleic acid sequence encoding the antisense compounds (e.g. antisense oligonucleotides, siRNA molecules, shRNA molecules) of the present disclosure may be administered in any suitable form, either as a liquid solution or suspension, as a solid form suitable for liquid solution or suspension in a liquid solution. The antisense compounds (e.g. antisense oligonucleotides, siRNA molecules, shRNA molecules) may be formulated with any appropriate and pharmaceutically acceptable excipient.

The vector, e.g., an AAV vector, comprising a nucleic acid sequence encoding the antisense compounds (e.g. antisense oligonucleotides, siRNA molecules, shRNA molecules) of the present disclosure may be administered in a “therapeutically effective” amount, i.e., an amount that is sufficient to alleviate and/or prevent at least one symptom associated with the disease, or provide improvement in the condition of the subject.

According to some embodiments, the vector, e.g., an AAV vector, may be administered to the CNS in a therapeutically effective amount to improve function and/or survival for a subject with ALS. As a non-limiting example, the vector may be administered intrathecally.

According to some embodiments, the vector, e.g., an AAV vector, may be administered to a subject (e.g., to the CNS of a subject via intrathecal administration) in a therapeutically effective amount for the antisense compounds (e.g. antisense oligonucleotides, siRNA molecules, shRNA molecules) to target the motor neurons and astrocytes in the spinal cord and/or brain steam. As a non-limiting example, the antisense compounds (e.g. antisense oligonucleotides, siRNA molecules, shRNA molecules) may reduce the expression of c9orf72 protein or mRNA.

According to some embodiments, the vector, e.g., an AAV vector, may be administered to a subject (e.g., to the CNS of a subject) in a therapeutically effective amount to slow the functional decline of a subject (e.g., determined using a known evaluation method such as the ALS functional rating scale (ALSFRS)) and/or prolong ventilator-independent survival of subjects (e.g., decreased mortality or need for ventilation support). As a non-limiting example, the vector may be administered intrathecally.

According to some embodiments, the vector, e.g., an AAV vector, may be administered to the cisterna magna in a therapeutically effective amount to transduce spinal cord motor neurons and/or astrocytes. As a non-limiting example, the vector may be administered intrathecally.

According to some embodiments, the vector, e.g., an AAV vector, may be administered using intrathecal infusion in a therapeutically effective amount to transduce spinal cord motor neurons and/or astrocytes. As a non-limiting example, the vector may be administered intrathecally.

According to some embodiments, the vector, e.g., an AAV vector, comprising antisense compounds (e.g. antisense oligonucleotides, siRNA molecules, shRNA molecules) may be formulated. As a non-limiting example the baricity and/or osmolality of the formulation may be optimized to ensure optimal drug distribution in the central nervous system or a region or component of the central nervous system.

According to some embodiments, the vector, e.g., an AAV vector, comprising antisense compounds (e.g. antisense oligonucleotides, siRNA molecules, shRNA molecules) may be delivered to a subject via a single route administration.

According to some embodiments, the vector, e.g., an AAV vector, comprising antisense compounds (e.g. antisense oligonucleotides, siRNA molecules, shRNA molecules) may be delivered to a subject via a multi-site route of administration. A subject may be administered the vector, e.g., an AAV vector, comprising antisense compounds (e.g. antisense oligonucleotides, siRNA molecules, shRNA molecules) at 2, 3, 4, 5 or more than 5 sites.

According to some embodiments, a subject may be administered the vector, e.g., an AAV vector, comprising antisense compounds (e.g. antisense oligonucleotides, siRNA molecules, shRNA molecules) described herein using a bolus infusion.

According to some embodiments, a subject may be administered the vector, e.g., an AAV vector, comprising antisense compounds (e.g. antisense oligonucleotides, siRNA molecules, shRNA molecules) described herein using sustained delivery over a period of minutes, hours or days. The infusion rate may be changed depending on the subject, distribution, formulation or another delivery parameter.

According to some embodiments, the catheter may be located at more than one site in the spine for multi-site delivery. The vector, e.g., an AAV vector, comprising antisense compounds (e.g. antisense oligonucleotides, siRNA molecules, shRNA molecules) may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

According to some embodiments, a subject may be analyzed for spinal anatomy and pathology prior to delivery of the vector, e.g., an AAV vector, comprising antisense compounds (e.g. antisense oligonucleotides, siRNA molecules, shRNA molecules) described herein. As a non-limiting example, a subject with scoliosis may have a different dosing regimen and/or catheter location compared to a subject without scoliosis.

According to some embodiments, the orientation of the spine of the subject during delivery of the vector, e.g., an AAV vector, comprising antisense compounds (e.g. antisense oligonucleotides, siRNA molecules, shRNA molecules) may be vertical to the ground.

According to some embodiments, the orientation of the spine of the subject during delivery of the vector, e.g., an AAV vector, comprising antisense compounds (e.g. antisense oligonucleotides, siRNA molecules, shRNA molecules) may be horizontal to the ground.

According to some embodiments, the spine of the subject may be at an angle as compared to the ground during the delivery of the vector, e.g., an AAV vector, comprising antisense compounds (e.g. antisense oligonucleotides, siRNA molecules, shRNA molecules). The angle of the spine of the subject as compared to the ground may be at least 10, 20, 30, 40, 50, 60, 70, 80, 90, 100, 110, 120, 130, 140, 150 or 180 degrees.

According to some embodiments, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

The pharmaceutical compositions of the present disclosure may be administered to a subject using any amount effective for reducing, preventing and/or treating a c9orf72 associated disorder (e.g., ALS). The exact amount required will vary from subject to subject, depending on the species, age, and general condition of the subject, the severity of the disease, the particular composition, its mode of administration, its mode of activity, and the like.

The compositions of the present disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutic effectiveness for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the siRNA duplexes employed; the duration of the treatment; drugs used in combination or coincidental with the specific compound employed; and like factors well known in the medical arts.

According to some embodiments, the age and sex of a subject may be used to determine the dose of the compositions of the present disclosure. As a non-limiting example, a subject who is older may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a younger subject. As another non-limiting example, a subject who is younger may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to an older subject. As yet another non-limiting example, a subject who is female may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a male subject. As yet another non-limiting example, a subject who is male may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a female subject.

According to some embodiments, the doses of AAV vectors for delivering antisense compounds (e.g. antisense oligonucleotides, siRNA molecules, shRNA molecules) of the present disclosure may be adapted dependent on the disease condition, the subject and the treatment strategy.

According to the methods of treatment of the present disclosure, the concentration of vector that is administered may differ depending on production method and may be chosen or optimized based on concentrations determined to be therapeutically effective for the particular route of administration. According to some embodiments, the concentration in vector genomes per milliliter (vg/ml) is selected from the group consisting of about 10⁸ vg/ml, about 10⁹ vg/ml, about 10¹⁰ vg/ml, about 10¹¹ vg/ml, about 10¹² vg/ml, about 10¹³ vg/ml, and about 10¹⁴ vg/ml. In some embodiments, the concentration is in the range of 10¹⁰ vg/ml-10¹⁴ vg/ml, for example 10¹⁰ vg/ml-10¹⁴ vg/ml, 0¹⁰ vg/ml-10¹³ vg/ml, 10¹⁰ vg/ml-10¹² vg/ml, 10¹⁰ vg/ml-10¹¹ vg/ml, 10¹¹ vg/ml-10¹⁴ vg/ml, 10¹¹ vg/ml-10¹³ vg/ml, 10¹¹ vg/ml-10¹² vg/ml, 10¹² vg/ml-10¹⁴ vg/ml, 10¹² vg/ml-10¹³ vg/ml, or 10¹³ vg/ml-10¹⁴ vg/ml, delivered by intracranial injection, or intra cisterna magna injection, or intrathecal injection, or intramuscular injection, or intravitreal injection in a volume between about 0.1 ml and about 10 ml, for example between about 0.1 ml and about 10 ml, between about 0.5 ml and about 10 ml, between about 1 ml and about 10 ml, between about 5 ml and about 10 ml, between about 0.1 ml and about 5.0 ml, between about 0.1 nil and about 2.0 ml, between about 0.1 ml and about 1.0 ml, between about 0.1 ml and about 0.8 nil, between about 0.1 ml and about 0.6 ml, between about 0.1 ml and about 0.4 ml, between about 0.1 ml and about 0.2 ml, between about 0.2 ml and about 1.0 ml, between about 0.2 ml and about 0.8 ml, between about 0.2 ml and about 0.6 ml, between about 0.2 ml and about 0.4 nil, between about 0.4 ml and about 1.0 ml, between about 0.4 ml and about 0.8 ml, between about 0.4 ml and about 0.6 ml, between about 0.6 ml and about 1.0 ml, between about 0.6 ml and about 0.8 ml, between about 0.8 ml and about 1.0 ml, or about 0.1 ml, about 0.2 ml, about 0.4 nil, about 0.6 ml, about 0.8 ml, and about 1.0 ml.

According to some embodiments, one or more additional therapeutic agents may be administered to the subject.

The effectiveness of the compositions described herein can be monitored by several criteria. For example, after treatment in a subject using methods of the present disclosure, the subject may be assessed for e.g., an improvement and/or stabilization and/or delay in the progression of one or more signs or symptoms of the disease state by one or more clinical parameters including those described herein. Examples of such tests are known in the art, and include objective as well as subjective (e.g., subject reported) measures.

### In Vitro Analysis

Inhibition of levels or expression of a c9orf72 nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real time PCR, by being run simultaneously with the target, multiplexing, or separately. Total RNA is quantified using RIBOGREEN RNA quantification reagent (Invetrogen, Inc. Eugene, Oreg.). Methods of RNA quantification by RIBOGREEN are taught in Jones, L. J., et al., (Analytical Biochemistry, 1998, 265, 368-374). A CYTOFLUOR 4000 instrument (PE Applied Biosystems) is used to measure RIBOGREEN fluorescence.

Probes and primers are designed to hybridize to a C9ORF72 nucleic acid. Methods for designing real-time PCR probes and primers are well known in the art, and may include the use of software such as PRIMER EXPRESS Software (Applied Biosystems, Foster City, Calif.).

### Analysis of Protein Levels

Antisense inhibition of c9orf72 nucleic acids can be assessed by measuring c9orf72 protein levels. Protein levels of c9orf72 can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of mouse, rat, monkey, and human c9orf72 are commercially available.

### In Vivo Analysis

Antisense compounds described herein are tested in animals to assess their ability to inhibit expression of c9orf72 and produce phenotypic changes, such as, improved motor function and respiration. According to some embodiments, motor function is measured by rotarod, grip strength, pole climb, open field performance, balance beam, hindpaw footprint testing in the animal. In certain embodiments, respiration is measured by whole body plethysmograph, invasive resistance, and compliance measurements in the animal. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from CNS tissue or CSF and changes in c9orf72 nucleic acid expression are measured.

### VI. Kits

The rAAV compositions as described herein may be contained within a kit designed for use in one of the methods of the disclosure as described herein. According to one embodiment, a kit of the disclosure comprises (a) any one of the vectors of the disclosure, and (b) instructions for use thereof. According to some embodiments, a vector of the disclosure may be any type of vector known in the art, including a non-viral or viral vector, as described supra. According to some embodiments, the vector is a viral vector, such as a vector derived from an adeno-associated virus, an adenovirus, a retrovirus, a lentivirus, a vaccinia/poxvirus, or a herpesvirus (e.g., herpes simplex virus (HSV)). According to preferred embodiments, the vector is an adeno-associated viral (AAV) vector.

According to some embodiments, the kits may further comprise instructions for use. According to some embodiments, the instructions for use include instructions according to one of the methods described herein. The instructions provided with the kit may describe how the vector can be administered for therapeutic purposes, e.g., for treating a c9orf72 associated disease (e.g. AML or FTD). According to some embodiments wherein the kit is to be used for therapeutic purposes, the instructions include details regarding recommended dosages and routes of administration.

According to some embodiments, the kits further contain buffers and/or pharmaceutically acceptable excipients. Additional ingredients may also be used, for example preservatives, buffers, tonicity agents, antioxidants and stabilizers, nonionic wetting or clarifying agents, viscosity-increasing agents, and the like. The kits described herein can be packaged in single unit dosages or in multidosage forms. The contents of the kits are generally formulated as sterile and substantially isotonic solution.

All patents and publications mentioned herein are incorporated herein by reference to the extend allowed by law for the purpose of describing and disclosing the proteins, enzymes, vectors, host cells, and methodologies reported therein that might be used with the present disclosure. However, nothing herein is to be construed as an admission that the disclosure is not entitled to antedate such disclosure by virtue of prior disclosure.

The present disclosure is further illustrated by the following examples, which should not be construed as further limiting. The contents of all figures and all references, patents and published patent applications cited throughout this application, as well as the Figures, are expressly incorporated herein by reference in their entirety.

### EXAMPLES

### Example 1. Methods

The invention was performed using, but not limited to, the following methods. The methods as described herein are set forth in PCT Application No. PCT/US2007/017645, filed on Aug. 8, 2007, entitled Recombinant AAV Production in Mammalian Cells, which claims the benefit of U.S. application Ser. No. 11/503,775, entitled Recombinant AAV Production in Mammalian Cells, filed Aug. 14, 2007, which is a continuation-in-part of U.S. application Ser. No. 10/252,182, entitled High Titer Recombinant AAV Production, filed Sep. 23, 2002, now U.S. Pat. No. 7,091,029, issued Aug. 15, 2006. The contents of all the aforementioned applications are hereby incorporated by reference in their entirety.

rHSV Co-Infection Method

The rHSV co-infection method for recombinant adeno-associated virus (rAAV) production employs two ICP27-deficient recombinant herpes simplex virus type 1 (rHSV-1) vectors, one bearing the AAV rep and cap genes (rHSV-rep2capX, with “capX” referring to any of the AAV serotypes), and the second bearing the gene of interest (GOI) cassette flanked by AAV inverted terminal repeats (ITRs). Although the system was developed with AAV serotype 2 rep, cap, and ITRs, as well as the humanized green fluorescent protein gene (GFP) as the transgene, the system can be employed with different transgenes and serotype/pseudotype elements.

Mammalian cells are infected with the rHSV vectors, providing all cis and trans-acting rAAV components as well as the requisite helper functions for productive rAAV infection. Cells are infected with a mixture of rHSV-rep2capX and rHSV-GOI. Cells are harvested and lysed to liberate rAAV-GOI, and the resulting vector stock is titered by the various methods described below.

DOC-Lysis

At harvest, cells and media are separated by centrifugation. The media is set aside while the cell pellet is extracted with lysis buffer (20 mM Tris-HCl, pH 8.0, 150 mM NaCl) containing 0.5% (w/v) deoxycholate (DOC) using 2 to 3 freeze-thaw cycles, which extracts cell-associated rAAV. In some instances, the media and cell-associated rAAV lysate is recombined.

In Situ Lysis

An alternative method for harvesting rAAV is by in situ lysis. At the time of harvest, MgCl₂ is added to a final concentration of 1 mM, 10% (v/v) Triton X-100 added to a final concentration of 1% (v/v), and Benzonase is added to a final concentration of 50 units/mL. This mixture is either shaken or stirred at 37° C. for 2 hours.

Quantitative Real-Time PCR to Determine DRP Yield

The DNAse-resistant particle (DRP) assay employs sequence-specific oligonucleotide primers and a dual-labeled hybridizing probe for detection and quantification of the amplified DNA sequence using real-time quantitative polymerase chain reaction (qPCR) technology. The target sequence is amplified in the presence of a fluorogenic probe which hybridizes to the DNA and emits a copy-dependent fluorescence. The DRP titer (DRP/mL) is calculated by direct comparison of relative fluorescence units (RFUs) of the test article to the fluorescent signal generated from known plasmid dilutions bearing the same DNA sequence. The data generated from this assay reflect the quantity of packaged viral DNA sequences, and are not indicative of sequence integrity or particle infectivity.

Green-Cell Infectivity Assay to Determine Infectious Particle Yield (rAA V-GFP Only)

Infectious particle (ip) titering is performed on stocks of rAA V-GFP using a green cell assay. C12 cells (a HeLa derived line that expressed AAV2 Rep and Cap genes—see references below) are infected with serial dilutions of rAA V-GFP plus saturating concentrations of adenovirus (to provide helper functions for AAV replication). After two to three days incubation, the number of fluorescing green cells (each cell representing one infectious event) are counted and used to calculate the ip/mL titer of the virus sample.

Clark K R et al. described recombinant adenoviral production in Hum. Gene Ther. 1995. 6:1329-1341 and Gene Ther. 1996. 3:1124-1132, both of which are incorporated by reference in their entireties herein.

TCID₅₀ to Determine rAAV Infectivity

Infectivity of rAAV particles harboring a gene of interest (rAAV-GOI) was determined using a tissue culture infectious dose at 50% (TCID₅₀) assay. Eight replicates of rAAV were serially diluted in the presence of human adenovirus type 5 and used to infect HeLaRC32 cells (a HeLa-derived cell line that expresses AAV2 rep and cap, purchased from ATCC) in a 96-well plate. At three days post-infection, lysis buffer (final concentrations of 1 mM Tris-HCl pH 8.0, 1 mM EDTA, 0.25% (w/v) deoxycholate, 0.45% (v/v) Tween-20, 0.1% (w/v) sodium dodecyl sulfate, 0.3 mg/mL Proteinase K) was added to each well then incubated at 37° C. for 1 h, 55° C. for 2 h, and 95° C. for 30 min. The lysate from each well (2.5 μL aliquot) was assayed in the DRP qPCR assay described above. Wells with Ct values lower than the value of the lowest quantity of plasmid of the standard curve were scored as positive. TCID₅₀ infectivity per mL (TCID₅₀/mL) was calculated based on the Karber equation using the ratios of positive wells at 10-fold serial dilutions.

Cell Lines and Viruses

Production of rAAV vectors for gene therapy is carried out in vitro, using suitable producer cell lines such as HEK293 cells (293). Other cell lines suitable for use in the invention include Vero, RD, BHK-21, HT-1080, A549, Cos-7, ARPE-19, and MRC-5.

Mammalian cell lines were maintained in Dulbecco's modified Eagle's medium (DMEM, Hyclone) containing 2-10% (v/v) fetal bovine serum (FBS, Hyclone) unless otherwise noted. Cell culture and virus propagation were performed at 37° C., 5% CO2 for the indicated intervals.

Infection Cell Density

Cells can be grown to various concentrations including, but not limited to at least about, at most about, or about 1×10⁶ to 4×10⁶ cells/mL. The cells can then be infected with recombinant herpesvirus at a predetermined MOI.

### Example 2. Multi-Variant (v1-NM-145005 & v2-NM-018325) c9orf72 Supplementation

Codon optimization of c9orf72 to avoid miRNA knock-down c9orf72 was codon optimized to avoid miRNA knock-down. The GenSmart v1.0 algorithm was used (genscript.com/tools/ensmart-codon-optimization). Greater than 50 permutations are performed. The restriction Enzyme sites (NotI (GCG|CCGC) & AscI (GGC|GCGCC)) were avoided. GC % was ranked, as shown in Table 2. High c9orf72 expression was preferably avoided, therefore according to some embodiments, three variants are enough for supplementation purposes.

The top candidates are shown in Table 2, below.

TABLE 2

| | | Avg GC % - | Excluded enzyme | | Avg GC % - |
|---|---|---|---|---|---|
| Gene name | Original sequence | Original | sites | Optimized sequence | Optimized |
| gene 14 | ATGTCGACTCTTTGCCCACCGCCATCT | 40.73% | NotI | ATGAGCACCCTGTGTCCTCCACCTAGCCCCGCCGTGGCCAAGACAGAGATCGC | 55.16% |
| | CCAGCTGTTGCCAAGACAGAGATTGCT | | [GCGGCCGC] | CCTGAGCGGAAAAAGCCCTCTGCTGGCCGCTACATTTGCCTACTGGGACAACA | |
| | TTAAGTGGCAAATCACCTTTATTAGCA | | | TCCTGGGCCCTAGAGTGCGGCACATTTGGGCCCCTAAGACCGAACAGGTGCTG | |
| | GCTACTTTTGCTTACTGGGACAATATT | | | CTGAGTGATGGAGAGATCACCTTCCTGGCTAATCACACCCTTAACGGCGAAAT | |
| | CTTGGTCCTAGAGTAAGGCACATTTGG | | | CCTGCGGAACGCCGAGAGCGGAGCCATCGACGTGAAGTTCTTCGTGTTAAGCG | |
| | GCTCCAAAGACAGAACAGGTACTTCTC | | | AGAAGGGCGTGATCATTGTGTCCCTGATCTTCGACGGCAACTGGAACGGCGAT | |
| | AGTGATGGAGAAATAACTTTTCTTGCC | | | AGATCTACATACGGCCTGTCCATCATTCTTCCACAGACAGAGCTGTCTTTCTA | |
| | AACCACACTCTAAATGGAGAAATCCTT | | | CCTGCCTCTGCACCGGGTGTGCGTGGACAGACTGACCCACATTATTAGAAAAG | |
| | CGAAATGCAGAGAGTGGTGCTATAGAT | | | GCAGAATCTGGATGCACAAGGAACGGCAGGAGAACGTGCAAAAGATCATCCTC | |
| | GTAAAGTTTTTTGTCTTGTCTGAAAAG | | | GAGGGTACAGAGAGAATGGAAGATCAGGGCCAGAGCATCATCCCCATGCTGAC | |
| | GGAGTGATTATTGTTTCATTAATCTTT | | | CGGCGAGGTGATCCCTGTGATGGAACTGCTGAGCAGCATGAAAAGCCACTCTG | |
| | GATGGAAACTGGAATGGGGATCGCAGC | | | TCCCCGAGGAAATCGACATCGCCGACACCGTGCTGAACGACGATGATATAGGA | |
| | ACATATGGACTATCAATTATACTTCCA | | | GATTCATGCCACGAGGGCTTCCTGCTGAATGCCATCAGCTCTCACCTGCAGAC | |
| | CAGACAGAACTTAGTTTCTACCTCCCA | | | CTGTGGCTGCAGCGTCGTGGTGGGCAGCAGCGCCGAGAAAGTGAACAAGATCG | |
| | CTTCATAGAGTGTGTGTTGATAGATTA | | | TGCGGACCCTGTGCCTGTTCCTGACCCCTGCTGAAAGAAAGTGCAGCAGACTG | |
| | ACACATATAATCCGGAAAGGAAGAATA | | | TGTGAAGCCGAATCTAGCTTTAAGTACGAGTCTGGACTGTTTGTGCAGGGCCT | |
| | TGGATGCATAAGGAAAGACAAGAAAAT | | | GCTGAAGGACAGCACAGGCTCCTTCGTGCTGCCCTTCAGACAGGTTATGTACG | |
| | GTCCAGAAGATTATCTTAGAAGGCACA | | | CCCCTTACCCCACCACCCACATCGATGTGGACGTCAACACAGTGAAGCAGATG | |
| | GAGAGAATGGAAGATCAGGGTCAGAGT | | | CCTCCTTGCCACGAGCACATCTACAACCAGCGTAGATACATGCGGAGCGAGCT | |
| | ATTATTCCAATGCTTACTGGAGAAGTG | | | GACCGCCTTTTGGCGGGCCACCTCTGAAGAGGACATGGCCCAGGATACAATCA | |
| | ATTCCTGTAATGGAACTGCTTTCATCT | | | TCTATACCGACGAGTCCTTCACCCCTGATCTGAATATCTTCCAAGACGTGCTT | |
| | ATGAAATCACACAGTGTTCCTGAAGAA | | | CATAGAGATACACTGGTGAAAGCCTTCCTCGACCAGGTGTTCCAGCTGAAGCC | |
| | ATAGATATAGCTGATACAGTACTCAAT | | | TGGCCTGAGCCTGAGGTCCACATTCCTCGCTCAGTTCCTGCTCGTGCTGCACA | |
| | GATGATGATATTGGTGACAGCTGTCAT | | | GAAAGGCCCTGACCCTTATCAAGTACATCGAGGATGACACCCAGAAGGGCAAG | |
| | GAAGGCTTTCTTCTCAATGCCATCAGC | | | AAGCCGTTCAAGTCCCTCAGAAACCTGAAAATCGACCTGGACCTGACAGCCGA | |
| | TCACACTTGCAAACCTGTGGCTGTTCC | | | GGGAGATCTGAACATCATCATGGCTCTGGCCGAAAAGATCAAGCCCGGCCTGC | |
| | GTTGTAGTAGGTAGCAGTGCAGAGAAA | | | ATTCTTTCATCTTCGGCAGACCTTTTTACACCAGCGTGCAAGAGCGGGACGTG | |
| | GTAAATAAGATAGTCAGAACATTATGC | | | CTGATGACATTCTGA (SEQ ID NO: 100) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SSEQ ID | | | | |
| | NO: 89) | | | | |
| gene 8 | ATGTCGACTCTTTGCCCACCGCCATCT | 40.73% | NotI | ATGAGCACCCTGTGCCCTCCACCTAGCCCCGCCGTGGCCAAGACAGAGATCGC | 55.65% |
| | CCAGCTGTTGCCAAGACAGAGATTGCT | | [GCGGCCGC] | CCTTTCTGGCAAGTCCCCACTGCTGGCCGCTACCTTCGCCTATTGGGACAACA | |
| | TTAAGTGGCAAATCACCTTTATTAGCA | | | TCTTGGGCCCCAGAGTGCGGCACATCTGGGCCCCTAAGACCGAGCAGGTGCTG | |
| | GCTACTTTTGCTTACTGGGACAATATT | | | CTGAGTGATGGCGAGATCACCTTCCTGGCTAATCACACCCTGAACGGCGAGAT | |
| | CTTGGTCCTAGAGTAAGGCACATTTGG | | | CCTGAGAAACGCCGAGAGCGGCGCCATCGACGTGAAATTCTTCGTGCTGAGCG | |
| | GCTCCAAAGACAGAACAGGTACTTCTC | | | AGAAAGGCGTGATCATCGTGTCCCTGATCTTCGACGGAAATTGGAACGGCGAC | |
| | AGTGATGGAGAAATAACTTTTCTTGCC | | | AGAAGCACCTACGGCCTGAGCATCATCCTCCCCCAGACCGAGCTGTCCTTCTA | |
| | AACCACACTCTAAATGGAGAAATCCTT | | | CCTGCCTCTGCATAGAGTGTGCGTGGACCGCCTGACACACATCATTAGAAAGG | |
| | CGAAATGCAGAGAGTGGTGCTATAGAT | | | GCAGAATCTGGATGCACAAGGAACGGCAGGAGAACGTGCAGAAAATTATCCTG | |
| | GTAAAGTTTTTTGTCTTGTCTGAAAAG | | | GAAGGTACAGAGAGAATGGAAGATCAGGGACAGTCTATCATCCCCATGCTGAC | |
| | GGAGTGATTATTGTTTCATTAATCTTT | | | CGGCGAAGTGATCCCTGTGATGGAACTGCTGTCTAGCATGAAGTCTCATTCTG | |
| | GATGGAAACTGGAATGGGGATCGCAGC | | | TGCCTGAGGAAATCGACATCGCCGACACCGTGCTGAACGACGACGACATCGGC | |
| | ACATATGGACTATCAATTATACTTCCA | | | GATAGCTGCCACGAGGGCTTCCTGCTGAACGCCATTAGCAGCCACCTGCAGAC | |
| | CAGACAGAACTTAGTTTCTACCTCCCA | | | CTGCGGATGTAGCGTGGTGGTCGGCAGCAGCGCCGAGAAGGTGAACAAGATCG | |
| | CTTCATAGAGTGTGTGTTGATAGATTA | | | TGCGGACACTGTGCCTGTTCCTCACACCTGCTGAAAGAAAGTGCAGCAGACTG | |
| | ACACATATAATCCGGAAAGGAAGAATA | | | TGTGAAGCCGAAAGCAGCTTTAAGTACGAGAGCGGCCTGTTCGTGCAAGGCCT | |
| | TGGATGCATAAGGAAAGACAAGAAAAT | | | GCTGAAGGACAGCACAGGCTCTTTTGTGCTGCCTTTCAGACAGGTGATGTACG | |
| | GTCCAGAAGATTATCTTAGAAGGCACA | | | CCCCTTACCCCACCACACACATTGACGTGGACGTGAACACCGTGAAGCAGATG | |
| | GAGAGAATGGAAGATCAGGGTCAGAGT | | | CCTCCTTGTCACGAGCACATCTACAACCAGAGAAGATACATGAGATCTGAGCT | |
| | ATTATTCCAATGCTTACTGGAGAAGTG | | | GACCGCCTTTTGGCGGGCCACCAGCGAAGAGGACATGGCCCAGGATACCATCA | |
| | ATTCCTGTAATGGAACTGCTTTCATCT | | | TCTACACTGATGAGAGCTTCACCCCTGATCTGAACATTTTCCAGGACGTGCTG | |
| | ATGAAATCACACAGTGTTCCTGAAGAA | | | CACAGAGATACCCTGGTGAAGGCCTTCCTGGACCAGGTCTTTCAGCTGAAACC | |
| | ATAGATATAGCTGATACAGTACTCAAT | | | TGGACTGAGCCTGCGGTCCACATTCCTGGCCCAATTTCTGCTGGTGCTGCACC | |
| | GATGATGATATTGGTGACAGCTGTCAT | | | GGAAGGCTCTGACTCTGATCAAGTATATCGAGGACGATACACAGAAGGGCAAA | |
| | GAAGGCTTTCTTCTCAATGCCATCAGC | | | AAGCCCTTCAAGAGCCTGAGAAATCTGAAGATCGATCTGGATCTGACAGCCGA | |
| | TCACACTTGCAAACCTGTGGCTGTTCC | | | GGGCGACCTGAATATCATCATGGCCCTGGCAGAAAAGATTAAGCCTGGCCTGC | |
| | GTTGTAGTAGGTAGCAGTGCAGAGAAA | | | ACAGCTTCATCTTCGGCCGTCCATTCTACACCTCTGTGCAGGAGCGGGACGTT | |
| | GTAAATAAGATAGTCAGAACATTATGC | | | CTCATGACCTTCTGA (SEQ ID NO: 101) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 20 | ATGTCGACTCTTTGCCCACCGCCATCT | 40.73% | NotI | ATGAGCACCCTTTGTCCTCCTCCATCTCCTGCCGTGGCCAAGACAGAAATCGC | 55.79% |
| | CCAGCTGTTGCCAAGACAGAGATTGCT | | [GCGGCCGC] | CCTGTCCGGCAAGTCCCCTCTGCTGGCTGCTACATTTGCCTACTGGGACAACA | |
| | TTAAGTGGCAAATCACCTTTATTAGCA | | | TCCTGGGACCTAGAGTTAGACACATCTGGGCCCCTAAGACCGAGCAGGTTCTG | |
| | GCTACTTTTGCTTACTGGGACAATATT | | | CTGAGTGATGGCGAGATAACATTCCTGGCCAACCACACCCTGAATGGAGAAAT | |
| | CTTGGTCCTAGAGTAAGGCACATTTGG | | | CCTGAGAAACGCCGAGAGCGGCGCCATCGATGTGAAGTTCTTCGTGCTGAGCG | |
| | GCTCCAAAGACAGAACAGGTACTTCTC | | | AGAAGGGCGTGATCATTGTGTCCCTGATCTTCGACGGCAACTGGAACGGCGAT | |
| | AGTGATGGAGAAATAACTTTTCTTGCC | | | AGATCTACATACGGCCTGTCCATCATCCTGCCCCAGACCGAGCTGAGCTTTTA | |
| | AACCACACTCTAAATGGAGAAATCCTT | | | CCTGCCTCTGCACAGAGTTTGTGTGGACAGACTGACTCACATTATCAGAAAGG | |
| | CGAAATGCAGAGAGTGGTGCTATAGAT | | | GAAGAATCTGGATGCACAAGGAAAGACAGGAGAACGTGCAGAAGATTATTCTG | |
| | GTAAAGTTTTTTGTCTTGTCTGAAAAG | | | GAAGGTACAGAGAGAATGGAAGATCAGGGCCAGAGCATCATCCCCATGCTGAC | |
| | GGAGTGATTATTGTTTCATTAATCTTT | | | CGGCGAGGTGATCCCTGTGATGGAACTGCTGAGCAGCATGAAAAGCCACAGCG | |
| | GATGGAAACTGGAATGGGGATCGCAGC | | | TGCCCGAGGAAATCGACATCGCCGACACAGTGCTGAATGATGACGACATCGGC | |
| | ACATATGGACTATCAATTATACTTCCA | | | GACAGCTGCCACGAGGGCTTCCTGCTGAACGCTATCAGCTCTCATCTGCAGAC | |
| | CAGACAGAACTTAGTTTCTACCTCCCA | | | ATGCGGCTGTAGCGTCGTGGTGGGCAGCTCCGCCGAGAAGGTGAACAAGATCG | |
| | CTTCATAGAGTGTGTGTTGATAGATTA | | | TGCGGACACTGTGCCTGTTCCTCACCCCTGCTGAACGGAAATGCTCTAGACTC | |
| | ACACATATAATCCGGAAAGGAAGAATA | | | TGCGAGGCCGAGAGCAGCTTCAAGTACGAGTCCGGCCTCTTCGTGCAAGGCCT | |
| | TGGATGCATAAGGAAAGACAAGAAAAT | | | GCTGAAAGACAGTACAGGCAGCTTCGTGCTGCCTTTCAGACAGGTCATGTACG | |
| | GTCCAGAAGATTATCTTAGAAGGCACA | | | CCCCTTACCCCACCACCCACATCGATGTGGACGTGAACACCGTGAAGCAGATG | |
| | GAGAGAATGGAAGATCAGGGTCAGAGT | | | CCTCCGTGCCACGAGCACATCTACAACCAGAGAAGATACATGCGGTCTGAACT | |
| | ATTATTCCAATGCTTACTGGAGAAGTG | | | GACAGCCTTTTGGCGGGCCACCAGCGAAGAGGACATGGCCCAGGACACCATCA | |
| | ATTCCTGTAATGGAACTGCTTTCATCT | | | TCTACACCGACGAGTCTTTCACCCCTGACCTGAATATCTTTCAGGATGTGCTG | |
| | ATGAAATCACACAGTGTTCCTGAAGAA | | | CACAGAGATACCCTGGTCAAGGCCTTCCTGGACCAGGTGTTCCAGCTGAAGCC | |
| | ATAGATATAGCTGATACAGTACTCAAT | | | TGGACTGTCTCTGCGGAGCACCTTCCTGGCCCAATTTCTTCTGGTGCTCCACC | |
| | GATGATGATATTGGTGACAGCTGTCAT | | | GGAAGGCCCTGACACTGATCAAGTACATCGAGGACGACACCCAGAAAGGAAAA | |
| | GAAGGCTTTCTTCTCAATGCCATCAGC | | | AAGCCGTTCAAGTCCCTGCGGAACCTGAAGATCGACCTGGATCTGACCGCCGA | |
| | TCACACTTGCAAACCTGTGGCTGTTCC | | | GGGCGACCTGAACATCATCATGGCCCTGGCTGAGAAAATCAAGCCTGGCCTGC | |
| | GTTGTAGTAGGTAGCAGTGCAGAGAAA | | | ACAGCTTCATCTTCGGCAGACCTTTCTACACCAGCGTGCAGGAGCGGGACGTG | |
| | GTAAATAAGATAGTCAGAACATTATGC | | | CTGATGACCTTCTGA (SEQ ID NO: 102) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 18 | ATGTCGACTCTTTGCCCACCGCCATCT | 40.73% | Not I | ATGAGCACACTGTGCCCCCCACCTTCTCCAGCCGTGGCCAAGACCGAGATCGC | 55.86% |
| | CCAGCTGTTGCCAAGACAGAGATTGCT | | [GCGGCCGC] | CCTTTCTGGCAAGAGCCCTCTGCTGGCCGCCACATTCGCCTACTGGGACAACA | |
| | TTAAGTGGCAAATCACCTTTATTAGCA | | | TCCTGGGCCCTAGAGTGCGGCACATCTGGGCCCCTAAGACCGAGCAGGTGCTG | |
| | GCTACTTTTGCTTACTGGGACAATATT | | | CTGAGTGATGGCGAAATAACATTCCTGGCTAATCACACCCTCAACGGAGAGAT | |
| | CTTGGTCCTAGAGTAAGGCACATTTGG | | | CCTGAGAAATGCCGAGAGCGGCGCCATCGACGTCAAGTTCTTCGTGCTGTCTG | |
| | GCTCCAAAGACAGAACAGGTACTTCTC | | | AAAAGGGCGTGATCATAGTTTCTCTGATCTTCGACGGCAACTGGAACGGCGAC | |
| | AGTGATGGAGAAATAACTTTTCTTGCC | | | AGAAGCACCTACGGCCTGTCCATCATCCTGCCCCAGACAGAACTGAGCTTTTA | |
| | AACCACACTCTAAATGGAGAAATCCTT | | | CCTGCCTCTGCACAGAGTGTGCGTGGACCGGCTGACCCACATCATTAGAAAGG | |
| | CGAAATGCAGAGAGTGGTGCTATAGAT | | | GCAGAATCTGGATGCACAAGGAACGGCAGGAGAACGTGCAGAAGATCATCCTG | |
| | GTAAAGTTTTTTGTCTTGTCTGAAAAG | | | GAAGGGACCGAAAGAATGGAAGATCAGGGCCAGAGCATCATTCCTATGCTGAC | |
| | GGAGTGATTATTGTTTCATTAATCTTT | | | AGGCGAGGTGATCCCCGTGATGGAACTGCTGAGCAGCATGAAGTCTCACTCTG | |
| | GATGGAAACTGGAATGGGGATCGCAGC | | | TCCCCGAGGAAATCGACATCGCCGACACTGTGCTCAACGACGACGATATCGGC | |
| | ACATATGGACTATCAATTATACTTCCA | | | GATAGCTGCCACGAGGGATTTCTGCTGAACGCCATTTCTAGCCACCTGCAGAC | |
| | CAGACAGAACTTAGTTTCTACCTCCCA | | | CTGTGGCTGCAGCGTGGTCGTGGGCAGCTCCGCCGAGAAGGTGAACAAGATCG | |
| | CTTCATAGAGTGTGTGTTGATAGATTA | | | TGCGGACCCTGTGCCTGTTTCTGACACCTGCTGAACGGAAGTGCAGTAGACTG | |
| | ACACATATAATCCGGAAAGGAAGAATA | | | TGTGAAGCCGAGAGCAGCTTCAAATACGAGAGCGGACTGTTCGTTCAAGGCCT | |
| | TGGATGCATAAGGAAAGACAAGAAAAT | | | GCTGAAGGACAGCACCGGAAGCTTCGTGCTGCCTTTCAGACAGGTGATGTACG | |
| | GTCCAGAAGATTATCTTAGAAGGCACA | | | CCCCrTACCCCACAACACACATTGATGTCGATGTGAACACAGTGAAACAGATG | |
| | GAGAGAATGGAAGATCAGGGTCAGAGT | | | CCTCCATGTCACGAGCACATCTACAACCAGAGGCGGTACATGAGAAGCGAGCT | |
| | ATTATTCCAATGCTTACTGGAGAAGTG | | | GACCGCCTTTTGGCGGGCCACCAGCGAGGAAGATATGGCCCAGGACACAATCA | |
| | ATTCCTGTAATGGAACTGCTTTCATCT | | | TCTACACTGATGAGTCCTTTACCCCTGATCTGAATATCTTCCAGGACGTGCTG | |
| | ATGAAATCACACAGTGTTCCTGAAGAA | | | CATAGAGACACCCTGGTGAAGGCCTTCCTGGACCAGGTGTTCCAGCTGAAGCC | |
| | ATAGATATAGCTGATACAGTACTCAAT | | | TGGACTCAGCCTGCGGAGCACCTTCCTCGCTCAGTTCCTGCTCGTGCTGCACA | |
| | GATGATGATATTGGTGACAGCTGTCAT | | | GAAAGGCCCTGACCCTGATCAAGTACATCGAGGACGACACCCAGAAAGGCAAA | |
| | GAAGGCTTTCTTCTCAATGCCATCAGC | | | AAGCCCTTCAAGTCCCTCAGAAACCTGAAAATCGACCTGGACCTGACCGCCGA | |
| | TCACACTTGCAAACCTGTGGCTGTTCC | | | AGGCGACCTGAACATCATCATGGCCCTGGCCGAGAAGATCAAACCTGGCCTGC | |
| | GTTGTAGTAGGTAGCAGTGCAGAGAAA | | | ACAGCTTCATCTTCGGCAGACCTTTCTACACCAGCGTGCAGGAGAGAGATGTG | |
| | GTAAATAAGATAGTCAGAACATTATGC | | | CTGATGACCTTTTGA (SEQ ID NO: 103) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 10 | ATGTCGACTCTTTGCCCACCGCCATCT | 40.73% | NotI | ATGAGCACCCTGTGCCCTCCACCTAGCCCTGCCGTGGCCAAGACAGAGATCGC | 55.99% |
| | CCAGCTGTTGCCAAGACAGAGATTGCT | | [GCGGCCGC] | ACTGTCCGGCAAGTCCCCACTGCTGGCCGCCACCTTCGCCTACTGGGACAACA | |
| | TTAAGTGGCAAATCACCTTTATTAGCA | | | TCCTGGGCCCTAGAGTGCGGCACATTTGGGCCCCTAAGACCGAGCAGGTGCTG | |
| | GCTACTTTTGCTTACTGGGACAATATT | | | CTGTCTGATGGCGAGATCACCTTCCTGGCTAATCACACCCTGAACGGCGAAAT | |
| | CTTGGTCCTAGAGTAAGGCACATTTGG | | | CCTGAGAAATGCCGAGAGCGGCGCCATCGACGTGAAGTTCTTCGTGCTGTCTG | |
| | GCTCCAAAGACAGAACAGGTACTTCTC | | | AGAAGGGCGTGATCATCGTGTCCCTGATCTTCGACGGCAACTGGAACGGCGAC | |
| | AGTGATGGAGAAATAACTTTTCTTGCC | | | CGGAGCACCTACGGCCTGAGCATCATCCTGCCTCAGACCGAACTGTCCTTTTA | |
| | AACCACACTCTAAATGGAGAAATCCTT | | | CCTGCCTCTGCACAGAGTGTGCGTGGACAGACTGACACACATCATCAGAAAGG | |
| | CGAAATGCAGAGAGTGGTGCTATAGAT | | | GCAGAATCTGGATGCACAAGGAAAGACAGGAGAACGTGCAGAAGATCATTCTG | |
| | GTAAAGTTTTTTGTCTTGTCTGAAAAG | | | GAAGGTACAGAAAGAATGGAAGATCAGGGCCAGAGCATCATTCCTATGCTGAC | |
| | GGAGTGATTATTGTTTCATTAATCTTT | | | CGGCGAGGTGATCCCCGTGATGGAACTGCTGAGCAGCATGAAAAGCCACAGCG | |
| | GATGGAAACTGGAATGGGGATCGCAGC | | | TCCCCGAGGAAATCGACATCGCTGATACCGTGCTGAACGACGACGATATCGGC | |
| | ACATATGGACTATCAATTATACTTCCA | | | GATAGCTGCCACGAGGGCTTCCTGCTGAACGCCATCAGCAGCCACCTGCAGAC | |
| | CAGACAGAACTTAGTTTCTACCTCCCA | | | CTGCGGCTGCAGCGTGGTCGTGGGCAGCTCCGCCGAGAAGGTGAACAAGATCG | |
| | CTTCATAGAGTGTGTGTTGATAGATTA | | | TGCGGACCCTGTGTCTGTTCCTGACCCCTGCTGAGAGAAAGTGCAGCAGACTG | |
| | ACACATATAATCCGGAAAGGAAGAATA | | | TGTGAAGCCGAGTCCTCCTTCAAATACGAGAGCGGATTGTTTGTGCAAGGACT | |
| | TGGATGCATAAGGAAAGACAAGAAAAT | | | CCTGAAGGACAGCACAGGCTCTTTCGTGCTGCCCTTCAGACAGGTGATGTACG | |
| | GTCCAGAAGATTATCTTAGAAGGCACA | | | CCCCTTACCCCACCACACACATTGACGTGGACGTCAACACAGTGAAACAGATG | |
| | GAGAGAATGGAAGATCAGGGTCAGAGT | | | CCTCCATGTCACGAGCACATCTACAACCAGAGACGGTACATGAGAAGCGAGCT | |
| | ATTATTCCAATGCTTACTGGAGAAGTG | | | GACCGCCTTTTGGCGGGCCACAAGCGAGGAAGATATGGCCCAAGATACAATCA | |
| | ATTCCTGTAATGGAACTGCTTTCATCT | | | TCTATACAGACGAGTCTTTCACCCCTGATCTGAATATCTTTCAGGACGTCCTG | |
| | ATGAAATCACACAGTGTTCCTGAAGAA | | | CACCGGGACACCCTGGTGAAGGCCTTCCTGGATCAGGTGTTCCAGCTGAAACC | |
| | ATAGATATAGCTGATACAGTACTCAAT | | | CGGCCTGTCTCTGCGGTCCACCTTCCTGGCCCAGTTCCTGCTGGTCCTGCATA | |
| | GATGATGATATTGGTGACAGCTGTCAT | | | GAAAAGCCCTGACCCTGATCAAGTACATCGAGGACGACACGCAGAAAGGAAAG | |
| | GAAGGCTTTCTTCTCAATGCCATCAGC | | | AAGCCCTTCAAGAGCCTTAGAAACCTGAAGATCGACCTGGACCTCACAGCCGA | |
| | TCACACTTGCAAACCTGTGGCTGTTCC | | | AGGCGACCTGAACATCATCATGGCTCTGGCCGAAAAAATCAAGCCTGGCCTGC | |
| | GTTGTAGTAGGTAGCAGTGCAGAGAAA | | | ATAGCTTCATCTTCGGCAGACCTTTCTACACCTCTGTCCAGGAGAGAGATGTG | |
| | GTAAATAAGATAGTCAGAACATTATGC | | | CTGATGACATTCTGA (SEQ ID NO: 104) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 1 | ATGTCGACTCTTTGCCCACCGCCATCT | 40.73% | NotI | ATGAGCACCCTCTGTCCTCCCCCCAGCCCTGCTGTGGCCAAGACAGAGATCGC | 56.06% |
| | CCAGCTGTTGCCAAGACAGAGATTGCT | | [GCGGCCGC] | CCTGTCTGGAAAGTCCCCTCTGCTGGCTGCTACATTCGCCTACTGGGACAACA | |
| | TTAAGTGGCAAATCACCTTTATTAGCA | | | TCCTGGGCCCCAGAGTGCGGCACATCTGGGCCCCTAAGACCGAGCAGGTGCTC | |
| | GCTACTTTTGCTTACTGGGACAATATT | | | CTGAGCGACGGCGAGATCACCTTCCTGGCTAATCACACCCTGAACGGCGAGAT | |
| | CTTGGTCCTAGAGTAAGGCACATTTGG | | | CCTGAGAAATGCCGAAAGCGGCGCCATCGACGTGAAGTTCTTCGTGCTGTCTG | |
| | GCTCCAAAGACAGAACAGGTACTTCTC | | | AGAAGGGCGTGATCATTGTGTCCCTGATCTTCGACGGCAACTGGAACGGCGAT | |
| | AGTGATGGAGAAATAACTTTTCTTGCC | | | AGATCTACATACGGCCTGAGCATCATCCTGCCTCAGACCGAGCTGTCCTTCTA | |
| | AACCACACTCTAAATGGAGAAATCCTT | | | CCTGCCTCTGCACAGAGTGTGCGTGGACAGACTGACACACATCATTAGAAAGG | |
| | CGAAATGCAGAGAGTGGTGCTATAGAT | | | GCAGGATCTGGATGCACAAGGAAAGACAGGAGAACGTGCAGAAGATCATCCTG | |
| | GTAAAGTTTTTTGTCTTGTCTGAAAAG | | | GAAGGGACCGAAAGAATGGAAGATCAGGGCCAGAGCATCATCCCTATGCTGAC | |
| | GGAGTGATTATTGTTTCATTAATCTTT | | | CGGCGAAGTGATCCCCGTGATGGAACTGCTGAGTTCCATGAAAAGCCACTCTG | |
| | GATGGAAACTGGAATGGGGATCGCAGC | | | TGCCCGAGGAAATCGACATCGCCGACACCGTGCTGAACGACGACGACATAGGA | |
| | ACATATGGACTATCAATTATACTTCCA | | | GATAGCTGCCATGAGGGCTTCCTGCTGAACGCCATCAGCAGCCACCTGCAGAC | |
| | CAGACAGAACTTAGTTTCTACCTCCCA | | | CTGCGGTTGTAGCGTGGTGGTGGGCTCTAGCGCCGAGAAGGTGAACAAGATCG | |
| | CTTCATAGAGTGTGTGTTGATAGATTA | | | TGCGGACCCTGTGCCTGTTCCTGACACCTGCCGAACGAAAATGCTCTAGACTG | |
| | ACACATATAATCCGGAAAGGAAGAATA | | | TGTGAAGCCGAGAGCAGCTTTAAGTACGAGAGCGGCCTGTTCGTGCAAGGCCT | |
| | TGGATGCATAAGGAAAGACAAGAAAAT | | | GCTTAAAGACAGCACCGGCAGCTTCGTTCTGCCATTCAGACAGGTGATGTACG | |
| | GTCCAGAAGATTATCTTAGAAGGCACA | | | CCCCTTACCCTACCACCCACATTGACGTCGACGTGAACACCGTGAAACAGATG | |
| | GAGAGAATGGAAGATCAGGGTCAGAGT | | | CCTCCTTGCCACGAGCACATCTACAACCAGAGAAGATACATGCGGAGCGAGTT | |
| | ATTATTCCAATGCTTACTGGAGAAGTG | | | GACCGCCTTCTGGCGGGCCACCAGCGAGGAAGATATGGCCCAGGACACCATCA | |
| | ATTCCTGTAATGGAACTGCTTTCATCT | | | TCTACACCGACGAGAGCTTCACCCCTGACCTGAACATCTTTCAGGATGTGCTG | |
| | ATGAAATCACACAGTGTTCCTGAAGAA | | | CATAGAGATACACTGGTGAAGGCCTTTCTCGACCAGGTTTTCCAGCTGAAGCC | |
| | ATAGATATAGCTGATACAGTACTCAAT | | | CGGCCTGAGCCTGCGGAGCACATTTCTGGCTCAATTTCTCCTGGTCCTGCACC | |
| | GATGATGATATTGGTGACAGCTGTCAT | | | GGAAAGCCCTGACACTGATCAAGTACATCGAGGATGACACCCAGAAAGGCAAA | |
| | GAAGGCTTTCTTCTCAATGCCATCAGC | | | AAGCCCTTCAAGAGCCTGAGAAACCTGAAGATCGACCTGGACCTGACCGCCGA | |
| | TCACACTTGCAAACCTGTGGCTGTTCC | | | GGGCGACCTTAATATCATCATGGCCCTGGCTGAAAAGATTAAGCCTGGCCTGC | |
| | GTTGTAGTAGGTAGCAGTGCAGAGAAA | | | ACAGCTTCATCTTCGGCAGACCTTTCTATACAAGCGTGCAGGAGCGGGACGTG | |
| | GTAAATAAGATAGTCAGAACATTATGC | | | CTGATGACATTCTGA (SEQ ID NO: 105) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 7 | ATGTCGACTCTTTGCCCACCGCCATCT | 40.73% | NotI | ATGAGCACACTGTGTCCTCCACCATCTCCTGCCGTGGCCAAGACCGAGATCGC | 56.06% |
| | CCAGCTGTTGCCAAGACAGAGATTGCT | | [GCGGCCGC] | CCTGAGCGGAAAAAGCCCCCTGCTGGCCGCTACCTTCGCCTACTGGGACAACA | |
| | TTAAGTGGCAAATCACCTTTATTAGCA | | | TCCTGGGCCCTAGAGTGCGGCACATCTGGGCCCCTAAGACAGAGCAGGTGCTC | |
| | GCTACTTTTGCTTACTGGGACAATATT | | | CTGAGTGATGGCGAGATAACATTCCTGGCTAATCACACCCTGAATGGCGAAAT | |
| | CTTGGTCCTAGAGTAAGGCACATTTGG | | | CCTGAGAAACGCCGAAAGTGGCGCCATTGACGTGAAGTTCTTCGTGCTGTCCG | |
| | GCTCCAAAGACAGAACAGGTACTTCTC | | | AGAAGGGCGTGATCATCGTGTCCCTGATCTTCGACGGCAACTGGAACGGCGAT | |
| | AGTGATGGAGAAATAACTTTTCTTGCC | | | AGAAGCACCTACGGCCTGTCTATCATCCTGCCTCAGACCGAGCTGAGCTTCTA | |
| | AACCACACTCTAAATGGAGAAATCCTT | | | CCTGCCTCTGCACAGAGTGTGCGTGGACAGACTGACACACATCATTAGAAAGG | |
| | CGAAATGCAGAGAGTGGTGCTATAGAT | | | GCAGAATCTGGATGCACAAGGAACGGCAGGAGAACGTGCAGAAAATCATCCTG | |
| | GTAAAGTTTTTTGTCTTGTCTGAAAAG | | | GAAGGGACCGAAAGGATGGAAGATCAGGGCCAGAGCATCATCCCCATGCTGAC | |
| | GGAGTGATTATTGTTTCATTAATCTTT | | | TGGAGAGGTGATCCCTGTTATGGAACTGCTGAGCAGCATGAAGAGCCACAGCG | |
| | GATGGAAACTGGAATGGGGATCGCAGC | | | TGCCCGAAGAGATTGACATCGCCGACACCGTGCTGAACGACGACGACATAGGA | |
| | ACATATGGACTATCAATTATACTTCCA | | | GATTCATGCCACGAAGGATTCCTGCTCAACGCCATCAGCAGCCACCTGCAGAC | |
| | CAGACAGAACTTAGTTTCTACCTCCCA | | | ATGCGGCTGCTCTGTGGTCGTGGGCAGCAGCGCCGAGAAAGTGAACAAGATCG | |
| | CTTCATAGAGTGTGTGTTGATAGATTA | | | TGCGGACCCTCTGTCTGTTTCTCACACCCGCTGAGCGGAAGTGCAGCAGACTG | |
| | ACACATATAATCCGGAAAGGAAGAATA | | | TGCGAGGCCGAGTCTAGCTTTAAGTACGAGAGCGGCCTGTTCGTGCAAGGCCT | |
| | TGGATGCATAAGGAAAGACAAGAAAAT | | | GCTGAAGGACTCTACCGGCTCCTTTGTGCTCCCTTTTAGACAGGTGATGTACG | |
| | GTCCAGAAGATTATCTTAGAAGGCACA | | | CCCCTTACCCCACCACCCACATTGATGTGGACGTCAACACCGTGAAACAGATG | |
| | GAGAGAATGGAAGATCAGGGTCAGAGT | | | CCTCCTTGCCACGAGCACATCTACAACCAGAGACGGTACATGCGGAGCGAGCT | |
| | ATTATTCCAATGCTTACTGGAGAAGTG | | | GACCGCCTTCTGGCGGGCCACCTCCGAGGAAGATATGGCCCAGGACACCATCA | |
| | ATTCCTGTAATGGAACTGCTTTCATCT | | | TCTATACTGATGAGTCTTTCACCCCTGATCTGAACATCTTTCAGGATGTGCTG | |
| | ATGAAATCACACAGTGTTCCTGAAGAA | | | CACCGGGACACCCTGGTGAAGGCTTTCCTCGACCAGGTGTTCCAGCTGAAACC | |
| | ATAGATATAGCTGATACAGTACTCAAT | | | TGGCCTCAGCCTCAGAAGCACATTCCTGGCCCAGTTCCTGCTCGTGCTCCATA | |
| | GATGATGATATTGGTGACAGCTGTCAT | | | GAAAGGCCCTGACACTGATCAAGTACATCGAGGATGATACACAGAAGGGCAAG | |
| | GAAGGCTTTCTTCTCAATGCCATCAGC | | | AAGCCTTTCAAGTCCCTGCGGAACCTGAAGATCGACCTGGACCTGACAGCCGA | |
| | TCACACTTGCAAACCTGTGGCTGTTCC | | | AGGCGACCTGAACATCATTATGGCCCTGGCCGAGAAGATCAAGCCCGGCCTGC | |
| | GTTGTAGTAGGTAGCAGTGCAGAGAAA | | | ATTCTTTCATCTTCGGCAGACCTTTCTACACCAGCGTGCAGGAGAGAGATGTT | |
| | GTAAATAAGATAGTCAGAACATTATGC | | | CTGATGACCTTCTGA (SEQ ID NO: 106) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 12 | ATGTCGACTCTTTGCCCACCGCCATCT | 40.73% | NotI | ATGAGCACACTGTGTCCTCCACCGAGCCCTGCCGTGGCCAAGACAGAGATCGC | 56.13% |
| | CCAGCTGTTGCCAAGACAGAGATTGCT | | [GCGGCCGC] | CCTGAGCGGCAAGTCCCCTCTGCTGGCCGCCACATTCGCCTACTGGGACAACA | |
| | TTAAGTGGCAAATCACCTTTATTAGCA | | | TCCTGGGACCTAGAGTTAGACACATTTGGGCCCCTAAGACCGAGCAGGTGCTG | |
| | GCTACTTTTGCTTACTGGGACAATATT | | | CTGAGTGATGGAGAGATCACCTTCCTGGCCAACCACACCCTGAACGGCGAGAT | |
| | CTTGGTCCTAGAGTAAGGCACATTTGG | | | CCTGAGAAATGCCGAGAGCGGCGCTATCGATGTGAAGTTCTTCGTGCTGTCTG | |
| | GCTCCAAAGACAGAACAGGTACTTCTC | | | AGAAGGGTGTTATCATCGTGTCCCTGATCTTCGACGGCAACTGGAACGGCGAT | |
| | AGTGATGGAGAAATAACTTTTCTTGCC | | | AGAAGCACCTACGGCCTGAGCATCATCCTGCCTCAGACCGAGCTGAGCTTCTA | |
| | AACCACACTCTAAATGGAGAAATCCTT | | | CCTGCCACTGCACAGAGTGTGCGTGGACAGACTGACACACATCATTAGAAAGG | |
| | CGAAATGCAGAGAGTGGTGCTATAGAT | | | GAAGAATCTGGATGCACAAGGAAAGACAGGAGAACGTGCAAAAGATCATCCTG | |
| | GTAAAGTTTTTTGTCTTGTCTGAAAAG | | | GAAGGTACAGAGCGGATGGAAGATCAGGGCCAGAGCATCATACCCATGCTGAC | |
| | GGAGTGATTATTGTTTCATTAATCTTT | | | AGGCGAAGTGATCCCCGTGATGGAACTCCTCAGCTCCATGAAAAGCCACAGCG | |
| | GATGGAAACTGGAATGGGGATCGCAGC | | | TGCCCGAGGAAATCGACATCGCCGACACCGTGCTGAATGACGACGACATCGGC | |
| | ACATATGGACTATCAATTATACTTCCA | | | GACAGCTGCCACGAAGGCTTCCTGCTGAACGCCATCAGCAGCCACCTGCAGAC | |
| | CAGACAGAACTTAGTTTCTACCTCCCA | | | ATGCGGCTGCAGCGTCGTGGTGGGCTCTTCTGCCGAGAAGGTGAACAAGATCG | |
| | CTTCATAGAGTGTGTGTTGATAGATTA | | | TGCGGACCCTGTGCCTGTTCCTGACACCTGCTGAGAGGAAGTGCAGCAGACTG | |
| | ACACATATAATCCGGAAAGGAAGAATA | | | TGTGAAGCCGAATCCAGCTTTAAGTACGAGTCTGGCCTGTTTGTGCAAGGCCT | |
| | TGGATGCATAAGGAAAGACAAGAAAAT | | | CCTGAAAGACTCCACCGGCAGCTTTGTGCTGCCTTTTAGACAGGTGATGTACG | |
| | GTCCAGAAGATTATCTTAGAAGGCACA | | | CCCCTTACCCCACCACCCACATCGACGTCGACGTGAACACCGTGAAGCAGATG | |
| | GAGAGAATGGAAGATCAGGGTCAGAGT | | | CCTCCGTGCCACGAGCACATCTACAACCAGCGGAGATACATGAGAAGCGAGCT | |
| | ATTATTCCAATGCTTACTGGAGAAGTG | | | GACCGCCTTCTGGCGGGCCACCAGCGAGGAAGATATGGCACAGGACACCATCA | |
| | ATTCCTGTAATGGAACTGCTTTCATCT | | | TCTACACCGACGAGAGCTTCACCCCTGACCTGAACATCTTCCAAGATGTGCTG | |
| | ATGAAATCACACAGTGTTCCTGAAGAA | | | CACCGGGACACCCTGGTGAAAGCCTTCCTGGATCAGGTCTTTCAGCTGAAACC | |
| | ATAGATATAGCTGATACAGTACTCAAT | | | CGGCCTGTCTCTGAGATCTACCTTCCTGGCCCAGTTCCTGCTTGTGCTGCATA | |
| | GATGATGATATTGGTGACAGCTGTCAT | | | GAAAGGCCCTGACGCTGATCAAGTACATCGAGGATGATACACAGAAAGGAAAA | |
| | GAAGGCTTTCTTCTCAATGCCATCAGC | | | AAGCCCTTCAAGAGCCTGCGGAACCTGAAGATCGACCTGGACCTGACTGCCGA | |
| | TCACACTTGCAAACCTGTGGCTGTTCC | | | GGGCGACCTGAACATCATCATGGCCCTGGCTGAAAAGATTAAGCCAGGCCTGC | |
| | GTTGTAGTAGGTAGCAGTGCAGAGAAA | | | ACTCCTTCATCTTTGGCAGACCTTTCTACACCTCCGTGCAGGAGAGAGATGTG | |
| | GTAAATAAGATAGTCAGAACATTATGC | | | CTGATGACCTTCTGA (SEQ ID NO: 21) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 16 | ATGTCGACTCTTTGCCCACCGCCATCT | 40.73% | NotI | ATGAGCACACTCTGTCCTCCCCCCAGCCCCGCCGTGGCCAAGACCGAGATCGC | 56.13% |
| | CCAGCTGTTGCCAAGACAGAGATTGCT | | [GCGGCCGC] | CCTGAGCGGAAAGTCCCCTCTGCTTGCTGCTACATTTGCCTACTGGGACAACA | |
| | TTAAGTGGCAAATCACCTTTATTAGCA | | | TCTTGGGCCCTAGAGTGCGGCACATCTGGGCCCCTAAGACCGAGCAGGTCCTG | |
| | GCTACTTTTGCTTACTGGGACAATATT | | | CTGAGTGATGGCGAAATCACCTTCCTGGCTAATCACACCCTGAACGGCGAGAT | |
| | CTTGGTCCTAGAGTAAGGCACATTTGG | | | CCTGAGAAACGCCGAGTCCGGCGCCATCGATGTGAAGTTCTTCGTGCTGTCTG | |
| | GCTCCAAAGACAGAACAGGTACTTCTC | | | AAAAGGGCGTGATCATTGTGTCCCTGATCTTCGACGGAAATTGGAACGGCGAT | |
| | AGTGATGGAGAAATAACTTTTCTTGCC | | | AGATCTACCTACGGCCTGTCTATCATCCTGCCTCAGACAGAGCTGAGCTTCTA | |
| | AACCACACTCTAAATGGAGAAATCCTT | | | CCTGCCCCTGCACAGAGTGTGCGTGGACCGGCTGACACACATTATCAGAAAGG | |
| | CGAAATGCAGAGAGTGGTGCTATAGAT | | | GCAGAATCTGGATGCACAAGGAACGCCAGGAGAACGTGCAGAAGATCATCCTG | |
| | GTAAAGTTTTTTGTCTTGTCTGAAAAG | | | GAAGGCACCGAGAGAATGGAAGATCAGGGCCAGAGCATCATCCCCATGCTGAC | |
| | GGAGTGATTATTGTTTCATTAATCTTT | | | CGGCGAGGTGATTCCTGTGATGGAACTGCTGAGCAGCATGAAAAGCCACTCCG | |
| | GATGGAAACTGGAATGGGGATCGCAGC | | | TCCCCGAGGAAATCGACATCGCAGATACCGTGCTGAACGACGATGACATCGGC | |
| | ACATATGGACTATCAATTATACTTCCA | | | GACAGCTGCCACGAGGGATTCCTCCTGAATGCCATCAGCTCTCACCTGCAGAC | |
| | CAGACAGAACTTAGTTTCTACCTCCCA | | | ATGCGGCTGTAGCGTCGTCGTGGGCAGCAGCGCCGAGAAAGTGAACAAGATCG | |
| | CTTCATAGAGTGTGTGTTGATAGATTA | | | TGCGGACACTGTGTCTGTTCCTCACACCTGCCGAAAGAAAGTGCAGCAGACTG | |
| | ACACATATAATCCGGAAAGGAAGAATA | | | TGCGAGGCCGAGTCTAGCTTCAAGTACGAGAGCGGCCTCTTCGTGCAGGGACT | |
| | TGGATGCATAAGGAAAGACAAGAAAAT | | | GCTGAAGGACAGCACCGGCTCTTTCGTGCTGCCTTTCAGACAGGTGATGTACG | |
| | GTCCAGAAGATTATCTTAGAAGGCACA | | | CCCCTTACCCCACCACCCACATCGACGTTGACGTGAACACCGTGAAACAGATG | |
| | GAGAGAATGGAAGATCAGGGTCAGAGT | | | CCCCCGTGCCATGAACACATCTACAACCAGCGGAGATACATGAGAAGCGAGCT | |
| | ATTATTCCAATGCTTACTGGAGAAGTG | | | GACCGCCTTCTGGCGGGCCACCAGCGAGGAAGATATGGCTCAGGATACCATCA | |
| | ATTCCTGTAATGGAACTGCTTTCATCT | | | TCTATACAGACGAGAGCTTCACCCCTGACCTGAACATCTTTCAGGACGTGCTG | |
| | ATGAAATCACACAGTGTTCCTGAAGAA | | | CATAGAGATACACTCGTGAAGGCCTTTCTGGATCAGGTTTTCCAGCTGAAGCC | |
| | ATAGATATAGCTGATACAGTACTCAAT | | | TGGCCTGAGCCTGAGATCCACCTTCCTGGCACAATTTCTGCTGGTGCTGCACC | |
| | GATGATGATATTGGTGACAGCTGTCAT | | | GGAAGGCCCTGACCCTGATCAAGTACATCGAGGACGACACACAGAAAGGCAAG | |
| | GAAGGCTTTCTTCTCAATGCCATCAGC | | | AAGCCCTTTAAGAGCCTGCGGAACCTGAAAATTGATCTGGACCTGACTGCCGA | |
| | TCACACTTGCAAACCTGTGGCTGTTCC | | | GGGCGACCTGAATATCATCATGGCCCTGGCCGAGAAGATCAAGCCTGGACTGC | |
| | GTTGTAGTAGGTAGCAGTGCAGAGAAA | | | ACTCTTTCATCTTCGGCAGACCTTTCTACACAAGCGTGCAAGAGCGGGACGTG | |
| | GTAAATAAGATAGTCAGAACATTATGC | | | CTGATGACCTTCTGA (SEQ ID NO: 22) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 2 | ATGTCGACTCTTTGCCCACCGCCATCT | 40.73% | NotI | ATGAGCACCCTGTGTCCTCCGCCCAGCCCTGCCGTGGCCAAGACCGAAATCGC | 56.20% |
| | CCAGCTGTTGCCAAGACAGAGATTGCT | | [GCGGCCGC] | CCTGAGCGGAAAAAGCCCCCTGCTGGCCGCCACCTTTGCCTACTGGGACAACA | |
| | TTAAGTGGCAAATCACCTTTATTAGCA | | | TCCTGGGCCCTAGAGTGCGGCACATCTGGGCCCCTAAGACCGAGCAGGTGCTG | |
| | GCTACTTTTGCTTACTGGGACAATATT | | | CTGAGCGACGGCGAGATAACATTCCTCGCTAATCACACACTGAACGGCGAAAT | |
| | CTTGGTCCTAGAGTAAGGCACATTTGG | | | CCTGAGAAATGCCGAAAGCGGCGCCATCGACGTTAAGTTCTTCGTGCTGTCTG | |
| | GCTCCAAAGACAGAACAGGTACTTCTC | | | AAAAGGGCGTGATCATCGTGTCCCTGATCTTCGACGGCAACTGGAACGGCGAT | |
| | AGTGATGGAGAAATAACTTTTCTTGCC | | | AGATCAACCTACGGCCTGAGCATCATCCTGCCTCAGACCGAGCTGTCTTTCTA | |
| | AACCACACTCTAAATGGAGAAATCCTT | | | CCTGCCTCTGCATAGAGTGTGCGTGGACAGACTGACACACATCATCAGAAAGG | |
| | CGAAATGCAGAGAGTGGTGCTATAGAT | | | GAAGAATCTGGATGCACAAGGAAAGACAGGAGAACGTGCAGAAGATCATTCTG | |
| | GTAAAGTTTTTTGTCTTGTCTGAAAAG | | | GAAGGTACAGAGAGAATGGAAGATCAGGGACAGAGCATCATTCCTATGCTGAC | |
| | GGAGTGATTATTGTTTCATTAATCTTT | | | TGGAGAGGTGATCCCCGTGATGGAACTGCTGAGCTCCATGAAAAGCCACTCTG | |
| | GATGGAAACTGGAATGGGGATCGCAGC | | | TTCCTGAGGAAATCGACATCGCCGACACCGTGCTGAACGACGACGATATTGGA | |
| | ACATATGGACTATCAATTATACTTCCA | | | GATAGCTGCCACGAGGGCTTCCTTCTGAACGCCATCAGCAGCCACCTGCAGAC | |
| | CAGACAGAACTTAGTTTCTACCTCCCA | | | ATGCGGCTGCAGCGTCGTGGTGGGCTCCAGCGCCGAGAAGGTGAACAAGATCG | |
| | CTTCATAGAGTGTGTGTTGATAGATTA | | | TGCGGACCCTGTGCCTGTTCCTGACCCCTGCTGAGCGGAAGTGCAGTAGACTG | |
| | ACACATATAATCCGGAAAGGAAGAATA | | | TGTGAAGCCGAGAGCAGCTTCAAGTACGAGTCCGGCCTGTTTGTGCAGGGCCT | |
| | TGGATGCATAAGGAAAGACAAGAAAAT | | | GCTGAAGGACAGCACAGGCAGCTTCGTGCTGCCCTTCAGACAAGTGATGTACG | |
| | GTCCAGAAGATTATCTTAGAAGGCACA | | | CCCCTTACCCCACCACCCACATCGACGTCGACGTGAACACCGTGAAGCAGATG | |
| | GAGAGAATGGAAGATCAGGGTCAGAGT | | | CCTCCATGTCACGAGCACATCTACAACCAGAGGCGGTACATGAGATCTGAGCT | |
| | ATTATTCCAATGCTTACTGGAGAAGTG | | | GACCGCCTTTTGGCGGGCCACAAGCGAGGAAGATATGGCCCAGGACACCATCA | |
| | ATTCCTGTAATGGAACTGCTTTCATCT | | | TCTACACCGACGAGTCTTTCACCCCTGATCTGAATATCTTTCAGGATGTCCTG | |
| | ATGAAATCACACAGTGTTCCTGAAGAA | | | CACCGGGACACACTGGTGAAGGCCTTCCTGGACCAGGTGTTCCAGCTGAAGCC | |
| | ATAGATATAGCTGATACAGTACTCAAT | | | CGGCCTGTCCCTGCGGAGCACCTTCCTGGCCCAATTTCTGCTCGTGCTTCACA | |
| | GATGATGATATTGGTGACAGCTGTCAT | | | GAAAGGCCCTGACACTGATCAAGTACATCGAGGACGACACCCAGAAAGGCAAG | |
| | GAAGGCTTTCTTCTCAATGCCATCAGC | | | AAGCCTTTCAAGTCCCTGCGCAACCTGAAAATCGATCTGGACCTGACCGCCGA | |
| | TCACACTTGCAAACCTGTGGCTGTTCC | | | GGGCGACCTGAACATCATCATGGCCCTTGCCGAGAAAATCAAACCTGGCCTGC | |
| | GTTGTAGTAGGTAGCAGTGCAGAGAAA | | | ACAGCTTCATCTTCGGCAGACCTTTTTATACCAGCGTGCAGGAGAGAGATGTG | |
| | GTAAATAAGATAGTCAGAACATTATGC | | | CTTATGACCTTCTGA (SEQ ID NO: 23) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 11 | ATGTCGACTCTTTGCCCACCGCCATCT | 40.73% | NotI | ATGAGCACCCTGTGTCCTCCACCATCTCCTGCCGTGGCCAAGACAGAGATCGC | 56.20% |
| | CCAGCTGTTGCCAAGACAGAGATTGCT | | [GCGGCCGC] | CCTGTCTGGCAAGTCACCTCTGCTGGCCGCTACATTCGCCTACTGGGACAACA | |
| | TTAAGTGGCAAATCACCTTTATTAGCA | | | TCCTTGGACCTAGAGTGCGGCACATCTGGGCCCCTAAGACCGAGCAGGTTCTG | |
| | GCTACTTTTGCTTACTGGGACAATATT | | | CTGAGCGACGGCGAGATAACATTTCTGGCCAACCACACACTTAATGGCGAGAT | |
| | CTTGGTCCTAGAGTAAGGCACATTTGG | | | CCTGAGAAACGCCGAGTCTGGCGCCATCGATGTGAAGTTCTTCGTGCTGTCCG | |
| | GCTCCAAAGACAGAACAGGTACTTCTC | | | AGAAGGGCGTGATCATCGTGTCCCTGATCTTCGACGGCAACTGGAACGGCGAC | |
| | AGTGATGGAGAAATAACTTTTCTTGCC | | | CGGTCTACCTACGGCCTGTCCATCATCCTGCCCCAGACAGAGCTGAGTTTCTA | |
| | AACCACACTCTAAATGGAGAAATCCTT | | | CCTGCCACTGCATAGAGTGTGCGTGGACAGACTGACACACATCATCAGAAAGG | |
| | CGAAATGCAGAGAGTGGTGCTATAGAT | | | GCAGAATCTGGATGCACAAGGAACGGCAGGAGAACGTGCAGAAGATCATCCTC | |
| | GTAAAGTTTTTTGTCTTGTCTGAAAAG | | | GAGGGCACCGAGCGGATGGAAGATCAGGGCCAGAGCATCATTCCTATGCTGAC | |
| | GGAGTGATTATTGTTTCATTAATCTTT | | | AGGCGAAGTGATCCCCGTGATGGAACTGCTGTCTAGCATGAAAAGCCACAGCG | |
| | GATGGAAACTGGAATGGGGATCGCAGC | | | TGCCGGAAGAGATCGACATCGCCGACACAGTGCTGAACGACGACGACATCGGC | |
| | ACATATGGACTATCAATTATACTTCCA | | | GATAGCTGCCACGAGGGCTTCCTCCTGAACGCCATCAGCTCCCACCTGCAGAC | |
| | CAGACAGAACTTAGTTTCTACCTCCCA | | | CTGCGGCTGCTCTGTGGTCGTGGGCTCTAGCGCCGAAAAGGTGAACAAGATCG | |
| | CTTCATAGAGTGTGTGTTGATAGATTA | | | TGCGGACCCTGTGCCTGTTCCTGACACCTGCTGAAAGAAAATGCAGCAGACTG | |
| | ACACATATAATCCGGAAAGGAAGAATA | | | TGTGAAGCCGAGAGCAGCTTCAAGTACGAGAGCGGCCTGTTCGTGCAGGGACT | |
| | TGGATGCATAAGGAAAGACAAGAAAAT | | | CCTGAAGGACAGCACAGGCAGCTTTGTGCTGCCTTTCAGACAGGTGATGTACG | |
| | GTCCAGAAGATTATCTTAGAAGGCACA | | | CCCCCTACCCCACCACCCACATCGACGTCGACGTGAACACCGTGAAACAGATG | |
| | GAGAGAATGGAAGATCAGGGTCAGAGT | | | CCTCCTTGTCACGAGCACATCTACAACCAGCGGAGATACATGAGAAGCGAGCT | |
| | ATTATTCCAATGCTTACTGGAGAAGTG | | | GACGGCCTTTTGGCGGGCCACTTCCGAGGAAGATATGGCTCAGGACACAATCA | |
| | ATTCCTGTAATGGAACTGCTTTCATCT | | | TCTACACTGATGAGTCCTTCACCCCTGATCTGAATATCTTTCAGGACGTGCTG | |
| | A7GAAATCACACAGTGTTCCTGAAGAA | | | CACAGAGATACCCTGGTGAAGGCCTTCCTGGATCAGGTCTTTCAGCTGAAGCC | |
| | ATAGATATAGCTGATACAGTACTCAAT | | | CGGCCTGTCTCTGAGAAGCACCTTCCTGGCCCAGTTCCTGCTTGTGCTGCACC | |
| | GATGATGATATTGGTGACAGCTGTCAT | | | GGAAGGCCCTGACCCTGATCAAGTACATCGAGGACGATACCCAGAAAGGAAAA | |
| | GAAGGCTTTCTTCTCAATGCCATCAGC | | | AAGCCTTTTAAGAGCCTGCGGAACCTGAAAATCGACCTGGACCTGACCGCCGA | |
| | TCACACTTGCAAACCTGTGGCTGTTCC | | | GGGAGATCTGAACATCATCATGGCCCTGGCTGAAAAGATTAAGCCTGGACTGC | |
| | GTTGTAGTAGGTAGCAGTGCAGAGAAA | | | ACAGCTTCATCTTCGGCAGACCTTTCTACACCAGCGTGCAAGAGCGGGACGTG | |
| | GTAAATAAGATAGTCAGAACATTATGC | | | CTGATGACCTTTTGA (SEQ ID NO: 24) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAG7ACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 13 | ATGTCGACTCTTTGCCCACCGCCATCT | 40.73% | Not I | ATGAGCACACTGTGCCCTCCACCGAGCCCTGCTGTGGCCAAGACAGAGATCGC | 56.20% |
| | CCAGCTGTTGCCAAGACAGAGATTGCT | | [GCGGCCGC] | CCTCTCTGGCAAGAGCCCCCTGTTGGCCGCCACATTCGCCTACTGGGACAACA | |
| | TTAAGTGGCAAATCACCTTTATTAGCA | | | TCCTGGGTCCTAGAGTGCGGCACATCTGGGCCCCTAAGACCGAGCAGGTGCTG | |
| | GCTACTTTTGCTTACTGGGACAATATT | | | CTGAGTGATGGAGAAATAACATTCCTGGCCAACCACACCCTGAACGGCGAAAT | |
| | CTTGGTCCTAGAGTAAGGCACATTTGG | | | CCTGAGAAACGCCGAGAGCGGTGCTATCGACGTGAAGTTCTTCGTGCTCAGCG | |
| | GCTCCAAAGACAGAACAGGTACTTCTC | | | AGAAGGGAGTGATCATCGTGTCCCTGATCTTCGACGGCAACTGGAACGGCGAC | |
| | AGTGATGGAGAAATAACTTTTCTTGCC | | | CGGAGCACCTACGGCCTGAGCATCATCCTGCCTCAGACCGAGCTGAGCTTTTA | |
| | AACCACACTCTAAATGGAGAAATCCTT | | | CCTGCCTCTGCACAGAGTGTGCGTGGACAGACTGACCCACATCATTAGAAAGG | |
| | CGAAATGCAGAGAGTGGTGCTATAGAT | | | GCAGAATCTGGATGCACAAGGAACGGCAGGAGAACGTGCAGAAGATCATCCTC | |
| | GTAAAGTTTTTTGTCTTGTCTGAAAAG | | | GAGGGTACAGAGAGAATGGAAGATCAGGGCCAGTCTATCATCCCTATGCTGAC | |
| | GGAGTGATTATTGTTTCATTAATCTTT | | | CGGCGAGGTGATCCCAGTGATGGAACTGCTGTCCAGCATGAAGAGTCACTCTG | |
| | GATGGAAACTGGAATGGGGATCGCAGC | | | TTCCTGAGGAAATCGACATCGCCGACACCGTGCTGAACGACGATGACATCGGC | |
| | ACATATGGACTATCAATTATACTTCCA | | | GATAGCTGCCACGAGGGCTTCCTGCTGAATGCCATCAGCAGCCACCTGCAGAC | |
| | CAGACAGAACTTAGTTTCTACCTCCCA | | | ATGCGGCTGTAGCGTGGTGGTCGGCAGCAGCGCCGAAAAAGTGAACAAGATCG | |
| | CTTCATAGAGTGTGTGTTGATAGATTA | | | TGCGGACCCTCTGTCTGTTCCTGACACCTGCCGAGCGCAAGTGCAGCAGACTG | |
| | ACACATATAATCCGGAAAGGAAGAATA | | | TGTGAAGCCGAATCCAGCTTCAAGTACGAGTCTGGACTCTTCGTGCAAGGCCT | |
| | TGGATGCATAAGGAAAGACAAGAAAAT | | | GCTGAAGGACAGCACCGGCTCTTTTGTGCTGCCCTTCAGACAGGTCATGTACG | |
| | GTCCAGAAGATTATCTTAGAAGGCACA | | | CCCCATACCCCACCACACACATTGATGTTGACGTCAACACCGTGAAGCAGATG | |
| | GAGAGAATGGAAGATCAGGGTCAGAGT | | | CCTCCGTGCCATGAGCACATCTACAACCAGCGGAGATACATGAGATCTGAGCT | |
| | ATTATTCCAATGCTTACTGGAGAAGTG | | | GACCGCCTTTTGGCGGGCCACCAGCGAAGAGGATATGGCTCAAGACACAATCA | |
| | ATTCCTGTAATGGAACTGCTTTCATCT | | | TCTATACTGATGAGAGCTTCACCCCTGATCTGAATATCTTTCAGGACGTGCTG | |
| | ATGAAATCACACAGTGTTCCTGAAGAA | | | CACCGAGACACCCTCGTGAAAGCCTTCCTGGACCAGGTGTTCCAGCTGAAACC | |
| | ATAGATATAGCTGATACAGTACTCAAT | | | TGGCCTGTCTCTGAGAAGCACCTTCCTCGCCCAGTTCCTGCTGGTGCTGCACA | |
| | GATGATGATATTGGTGACAGCTGTCAT | | | GAAAGGCCCTGACACTGATCAAGTACATCGAGGACGACACCCAGAAAGGCAAG | |
| | GAAGGCTTTCTTCTCAATGCCATCAGC | | | AAACCCTTTAAGTCCCTGCGGAATCTGAAGATTGACCTGGATCTGACCGCCGA | |
| | TCACACTTGCAAACCTGTGGCTGTTCC | | | GGGCGACCTGAACATCATCATGGCCCTGGCCGAGAAGATCAAGCCCGGCCTCC | |
| | GTTGTAGTAGGTAGCAGTGCAGAGAAA | | | ACAGCTTCATCTTTGGCAGACCTTTCTACACCAGCGTGCAGGAGAGAGATGTG | |
| | GTAAATAAGATAGTCAGAACATTATGC | | | CTGATGACCTTCTGA (SEQ ID NO: 25) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 17 | ATGTCGACTCTTTGCCCACCGCCATCT | 40.73% | NotI | ATGAGCACCCTGTGTCCTCCACCGAGCCCTGCTGTGGCCAAGACCGAGATCGC | 56.20% |
| | CCAGCTGTTGCCAAGACAGAGATTGCT | | [GCGGCCGC] | CCTGAGCGGCAAATCTCCTCTGCTGGCCGCTACATTCGCCTACTGGGACAACA | |
| | TTAAGTGGCAAATCACCTTTATTAGCA | | | TCCTGGGCCCTAGAGTGCGGCACATTTGGGCCCCTAAGACCGAGCAGGTGCTG | |
| | GCTACTTTTGCTTACTGGGACAATATT | | | CTGAGCGACGGCGAAATCACCTTTCTGGCCAACCACACCCTGAACGGCGAGAT | |
| | CTTGGTCCTAGAGTAAGGCACATTTGG | | | CCTGCGGAACGCCGAAAGCGGCGCCATCGACGTCAAGTTCTTCGTGCTGTCTG | |
| | GCTCCAAAGACAGAACAGGTACTTCTC | | | AGAAGGGCGTGATCATTGTGTCCCTGATCTTCGACGGCAACTGGAACGGCGAC | |
| | AGTGATGGAGAAATAACTTTTCTTGCC | | | AGAAGCACCTACGGCCTGTCCATCATACTGCCCCAGACCGAGCTGTCTTTCTA | |
| | AACCACACTCTAAATGGAGAAATCCTT | | | CCTGCCTCTGCACCGCGTGTGCGTGGATAGACTGACCCACATCATTAGAAAAG | |
| | CGAAATGCAGAGAGTGGTGCTATAGAT | | | GCAGAATCTGGATGCACAAGGAACGGCAGGAGAACGTGCAGAAGATCATCCTG | |
| | GTAAAGTTTTTTGTCTTGTCTGAAAAG | | | GAAGGGACCGAAAGAATGGAAGATCAGGGACAGAGCATCATCCCCATGCTGAC | |
| | GGAGTGATTATTGTTTCATTAATCTTT | | | TGGCGAGGTGATCCCTGTGATGGAACTGCTGAGCTCTATGAAAAGCCACAGCG | |
| | GATGGAAACTGGAATGGGGATCGCAGC | | | TGCCCGAGGAAATCGATATCGCTGATACCGTGCTGAACGACGATGACATCGGC | |
| | ACATATGGACTATCAATTATACTTCCA | | | GATAGCTGCCACGAGGGCTTCCTGCTGAACGCCATCAGCAGCCACCTGCAGAC | |
| | CAGACAGAACTTAGTTTCTACCTCCCA | | | ATGCGGCTGTAGCGTCGTGGTGGGCTCTTCCGCCGAGAAGGTGAACAAGATCG | |
| | CTTCATAGAGTGTGTGTTGATAGATTA | | | TGCGGACCCTGTGCCTGTTCCTGACACCTGCCGAGAGAAAGTGCAGCAGACTG | |
| | ACACATATAATCCGGAAAGGAAGAATA | | | TGCGAGGCCGAATCTTCTTTTAAGTACGAGAGCGGACTCTTCGTGCAAGGACT | |
| | TGGATGCATAAGGAAAGACAAGAAAAT | | | GCTGAAAGACAGCACAGGCAGCTTTGTGCTGCCTTTCAGACAGGTTATGTACG | |
| | GTCCAGAAGATTATCTTAGAAGGCACA | | | CCCCCTACCCCACCACCCACATCGACGTGGACGTGAACACCGTGAAGCAGATG | |
| | GAGAGAATGGAAGATCAGGGTCAGAGT | | | CCTCCATGTCACGAGCACATCTACAACCAGCGGAGATACATGAGATCTGAACT | |
| | ATTATTCCAATGCTTACTGGAGAAGTG | | | GACCGCATTCTGGCGGGCCACCAGCGAAGAGGATATGGCCCAGGACACAATCA | |
| | ATTCCTGTAATGGAACTGCTTTCATCT | | | TCTATACAGACGAGAGCTTCACCCCTGATCTTAATATCTTCCAAGACGTGCTG | |
| | ATGAAATCACACAGTGTTCCTGAAGAA | | | CACCGGGACACCCTGGTGAAAGCCTTCCTGGATCAAGTGTTCCAGCTGAAGCC | |
| | ATAGATATAGCTGATACAGTACTCAAT | | | CGGCCTGAGCCTGAGATCCACATTCCTTGCTCAGTTCCTGCTGGTCCTGCACA | |
| | GATGATGATATTGGTGACAGCTGTCAT | | | GAAAGGCCCTGACGCTGATCAAGTACATCGAGGACGACACCCAGAAAGGCAAG | |
| | GAAGGCTTTCTTCTCAATGCCATCAGC | | | AAGCCTTTCAAGAGCCTGAGAAACCTGAAGATCGACCTGGACCTGACAGCCGA | |
| | TCACACTTGCAAACCTGTGGCTGTTCC | | | GGGCGACCTGAATATCATCATGGCCCTGGCTGAAAAGATCAAGCCTGGACTGC | |
| | GTTGTAGTAGGTAGCAGTGCAGAGAAA | | | ATAGCTTCATCTTTGGAAGACCTTTTTACACCTCCGTCCAAGAGCGGGACGTG | |
| | GTAAATAAGATAGTCAGAACATTATGC | | | CTGATGACCTTCTGA (SEQ ID NO: 26) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 19 | ATGTCGACTCTTTGCCCACCGCCATCT | 40.73% | NotI | ATGAGCACACTGTGCCCTCCTCCAAGCCCTGCCGTGGCCAAGACCGAGATAGC | 56.20% |
| | CCAGCTGTTGCCAAGACAGAGATTGCT | | [GCGGCCGC] | TCTGAGCGGCAAGAGCCCCCTGCTTGCCGCCACATTCGCCTACTGGGACAACA | |
| | TTAAGTGGCAAATCACCTTTATTAGCA | | | TCCTGGGCCCCAGAGTGCGGCACATCTGGGCCCCTAAGACAGAGCAGGTGCTG | |
| | GCTACTTTTGCTTACTGGGACAATATT | | | CTGAGCGACGGCGAGATCACCTTCCTGGCCAACCACACCCTGAATGGCGAAAT | |
| | CTTGGTCCTAGAGTAAGGCACATTTGG | | | CCTGAGAAACGCCGAGAGCGGTGCTATCGATGTGAAGTTCTTCGTGTTGTCTG | |
| | GCTCCAAAGACAGAACAGGTACTTCTC | | | AAAAGGGCGTGATCATAGTTTCTCTGATCTTTGATGGCAACTGGAACGGCGAT | |
| | AGTGATGGAGAAATAACTTTTCTTGCC | | | AGATCCACATACGGCCTCTCCATCATACTCCCCCAGACAGAGCTGAGCTTCTA | |
| | AACCACACTCTAAATGGAGAAATCCTT | | | TCTGCCTCTGCACAGAGTGTGCGTGGACAGACTGACCCACATCATTAGAAAGG | |
| | CGAAATGCAGAGAGTGGTGCTATAGAT | | | GCAGAATCTGGATGCACAAGGAACGGCAGGAGAACGTGCAAAAGATCATCCTG | |
| | GTAAAGTTTTTTGTCTTGTCTGAAAAG | | | GAAGGTACAGAGCGGATGGAAGATCAGGGCCAGTCTATCATTCCTATGCTGAC | |
| | GGAGTGATTATTGTTTCATTAATCTTT | | | CGGCGAGGTGATCCCCGTGATGGAACTGCTGTCTAGCATGAAATCCCACAGCG | |
| | GATGGAAACTGGAATGGGGATCGCAGC | | | TGCCGGAAGAAATCGACATCGCCGACACCGTGCTGAACGACGATGACATAGGA | |
| | ACATATGGACTATCAATTATACTTCCA | | | GATAGCTGCCACGAGGGCTTCCTGCTGAATGCCATCAGCAGCCACCTGCAGAC | |
| | CAGACAGAACTTAGTTTCTACCTCCCA | | | CTGCGGCTGCAGCGTGGTGGTCGGCAGCTCCGCCGAAAAGGTGAACAAGATCG | |
| | CTTCATAGAGTGTGTGTTGATAGATTA | | | TGCGGACCCTCTGTCTGTTCCTGACCCCTGCTGAAAGAAAGTGCAGTAGACTG | |
| | ACACATATAATCCGGAAAGGAAGAATA | | | TGTGAAGCCGAGAGCTCTTTTAAGTACGAGTCTGGACTTTTCGTGCAGGGCCT | |
| | TGGATGCATAAGGAAAGACAAGAAAAT | | | GCTGAAGGACAGCACAGGCAGCTTCGTGCTGCCTTTTAGACAGGTGATGTACG | |
| | GTCCAGAAGATTATCTTAGAAGGCACA | | | CCCCTTACCCCACCACCCACATCGACGTGGACGTCAACACCGTGAAACAGATG | |
| | GAGAGAATGGAAGATCAGGGTCAGAGT | | | CCTCCTTGCCATGAGCACATCTACAACCAGAGACGGTACATGAGAAGCGAGCT | |
| | ATTATTCCAATGCTTACTGGAGAAGTG | | | GACCGCCTTCTGGCGGGCCACCAGTGAAGAGGACATGGCACAGGATACCATCA | |
| | ATTCCTGTAATGGAACTGCTTTCATCT | | | TCTATACAGACGAGTCCTTCACCCCTGACCTGAACATCTTCCAGGACGTGCTG | |
| | ATGAAATCACACAGTGTTCCTGAAGAA | | | CACAGAGATACCCTGGTCAAGGCTTTTCTGGACCAGGTTTTCCAGCTGAAGCC | |
| | ATAGATATAGCTGATACAGTACTCAAT | | | TGGCCTGAGCCTGCGGTCCACCTTCCTGGCCCAGTTCCTGCTGGTGCTGCACC | |
| | GATGATGATATTGGTGACAGCTGTCAT | | | GGAAGGCCCTGACCCTCATCAAGTACATCGAGGACGACACCCAGAAAGGCAAA | |
| | GAAGGCTTTCTTCTCAATGCCATCAGC | | | AAGCCTTTCAAGTCCCTGCGCAACCTGAAAATTGACCTGGATCTGACAGCCGA | |
| | TCACACTTGCAAACCTGTGGCTGTTCC | | | GGGAGATCTGAATATCATCATGGCCCTGGCCGAGAAGATCAAGCCCGGCCTGC | |
| | GTTGTAGTAGGTAGCAGTGCAGAGAAA | | | ATAGCTTCATCTTCGGCCGCCCCTTTTACACCAGCGTGCAGGAGAGGGACGTG | |
| | GTAAATAAGATAGTCAGAACATTATGC | | | CTGATGACATTCTGA (SEQ ID NO: 27) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 3 | ATGTCGACTCTTTGCCCACCGCCATCT | 40.73% | NotI | ATGAGCACACTGTGTCCTCCACCTAGCCCTGCCGTGGCCAAGACCGAAATCGC | 56.27% |
| | CCAGCTGTTGCCAAGACAGAGATTGCT | | [GCGGCCGC] | CCTGAGCGGAAAGAGCCCCCTGCTGGCCGCCACCTTCGCCTACTGGGACAACA | |
| | TTAAGTGGCAAATCACCTTTATTAGCA | | | TCCTGGGCCCTAGAGTGCGGCACATCTGGGCCCCTAAGACCGAGCAGGTCTTG | |
| | GCTACTTTTGCTTACTGGGACAATATT | | | CTTTCTGATGGCGAAATCACCTTCCTCGCTAATCACACCCTGAACGGCGAGAT | |
| | CTTGGTCCTAGAGTAAGGCACATTTGG | | | CCTGAGAAATGCCGAGTCCGGCGCCATTGACGTGAAGTTCTTCGTGCTGAGCG | |
| | GCTCCAAAGACAGAACAGGTACTTCTC | | | AGAAGGGCGTGATCATCGTGTCCCTGATCTTCGACGGAAACTGGAACGGCGAC | |
| | AGTGATGGAGAAATAACTTTTCTTGCC | | | AGAAGCACCTACGGCCTGTCCATCATCCTGCCTCAGACCGAGCTGAGCTTCTA | |
| | AACCACACTCTAAATGGAGAAATCCTT | | | CCTGCCACTGCATAGAGTGTGCGTGGACCGGCTGACACACATCATCCGGAAGG | |
| | CGAAATGCAGAGAGTGGTGCTATAGAT | | | GCAGAATCTGGATGCACAAGGAACGGCAGGAGAACGTGCAGAAAATCATCCTG | |
| | GTAAAGTTTTTTGTCTTGTCTGAAAAG | | | GAAGGTACAGAGAGAATGGAAGATCAGGGCCAGAGCATCATCCCTATGCTGAC | |
| | GGAGTGATTATTGTTTCATTAATCTTT | | | CGGCGAGGTGATCCCCGTGATGGAACTGCTCAGCTCTATGAAGTCCCACAGCG | |
| | GATGGAAACTGGAATGGGGATCGCAGC | | | TGCCTGAGGAAATTGACATCGCCGATACCGTGCTGAACGACGACGACATCGGC | |
| | ACATATGGACTATCAATTATACTTCCA | | | GACAGCTGCCACGAGGGCTTCCTGCTGAACGCCATCAGCAGCCACCTGCAGAC | |
| | CAGACAGAACTTAGTTTCTACCTCCCA | | | CTGCGGCTGCAGCGTGGTGGTCGGCAGCTCCGCCGAGAAGGTGAACAAGATCG | |
| | CTTCATAGAGTGTGTGTTGATAGATTA | | | TGCGGACCCTCTGTCTGTTCCTGACTCCTGCTGAAAGAAAGTGCAGTAGACTG | |
| | ACACATATAATCCGGAAAGGAAGAATA | | | TGCGAGGCCGAATCTAGCTTCAAGTACGAGAGCGGCCTTTTTGTGCAGGGACT | |
| | TGGATGCATAAGGAAAGACAAGAAAAT | | | CCTGAAGGACTCTACAGGCTCTTTCGTGCTGCCTTTTAGACAGGTGATGTACG | |
| | GTCCAGAAGATTATCTTAGAAGGCACA | | | CCCCCTACCCCACCACCCACATTGACGTGGATGTCAACACAGTGAAACAGATG | |
| | GAGAGAATGGAAGATCAGGGTCAGAGT | | | CCCCCCTGCCACGAGCACATCTACAACCAGAGGCGGTACATGCGGAGCGAGCT | |
| | ATTATTCCAATGCTTACTGGAGAAGTG | | | GACCGCCTTCTGGCGGGCCACAAGCGAAGAGGACATGGCTCAAGACACCATCA | |
| | ATTCCTGTAATGGAACTGCTTTCATCT | | | TATATACAGACGAGAGCTTCACCCCTGATCTGAATATCTTTCAGGACGTGCTG | |
| | ATGAAATCACACAGTGTTCCTGAAGAA | | | CACCGGGACACCCTGGTCAAGGCCTTTCTGGACCAGGTGTTCCAGCTGAAACC | |
| | ATAGATATAGCTGATACAGTACTCAAT | | | TGGCCTGAGCCTGAGGTCCACCTTCTTGGCACAGTTCCTGCTGGTGCTGCACA | |
| | GATGATGATATTGGTGACAGCTGTCAT | | | GAAAAGCCCTGACACTGATCAAATACATCGAGGATGACACACAGAAGGGAAAA | |
| | GAAGGCTTTCTTCTCAATGCCATCAGC | | | AAGCCCTTCAAGTCTCTGAGAAACCTGAAGATCGATCTGGATCTGACAGCCGA | |
| | TCACACTTGCAAACCTGTGGCTGTTCC | | | GGGAGATCTGAACATCATCATGGCCCTGGCTGAAAAGATCAAGCCTGGACTTC | |
| | GTTGTAGTAGGTAGCAGTGCAGAGAAA | | | ATTCTTTCATCTTCGGCAGACCTTTCTACACCAGCGTGCAGGAGCGGGACGTT | |
| | GTAAATAAGATAGTCAGAACATTATGC | | | CTGATGACCTTTTGA (SEQ ID NO: 28) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 6 | ATGTCGACTCTTTGCCCACCGCCATCT | 40.73% | NotI | ATGAGCACCCTGTGCCCCCCCCCCAGCCCTGCCGTGGCCAAGACCGAGATCGC | 56.27% |
| | CCAGCTGTTGCCAAGACAGAGATTGCT | | [GCGGCCGC] | CCTCTCCGGCAAGTCCCCTCTGCTGGCCGCTACATTTGCCTACTGGGACAACA | |
| | TTAAGTGGCAAATCACCTTTATTAGCA | | | TCCTCGGCCCTAGAGTGCGGCACATTTGGGCCCCTAAGACCGAACAGGTCCTC | |
| | GCTACTTTTGCTTACTGGGACAATATT | | | CTGAGCGACGGCGAAATAACATTTCTGGCCAACCACACCCTGAACGGCGAAAT | |
| | CTTGGTCCTAGAGTAAGGCACATTTGG | | | CCTGAGAAACGCCGAGAGCGGCGCCATCGACGTGAAGTTCTTCGTGCTGTCCG | |
| | GCTCCAAAGACAGAACAGGTACTTCTC | | | AGAAAGGCGTGATCATCGTGTCCCTGATCTTCGACGGCAACTGGAACGGAGAT | |
| | AGTGATGGAGAAATAACTTTTCTTGCC | | | AGAAGCACATACGGACTGAGCATCATCCTCCCACAGACCGAGCTGTCTTTCTA | |
| | AACCACACTCTAAATGGAGAAATCCTT | | | CCTGCCTCTGCACCGGGTGTGCGTGGACAGACTGACCCACATCATTAGAAAGG | |
| | CGAAATGCAGAGAGTGGTGCTATAGAT | | | GCAGAATCTGGATGCACAAGGAACGGCAGGAGAACGTGCAAAAGATCATCCTG | |
| | GTAAAGTTTTTTGTCTTGTCTGAAAAG | | | GAAGGGACCGAGCGTATGGAAGATCAGGGCCAGAGCATCATTCCTATGCTGAC | |
| | GGAGTGATTATTGTTTCATTAATCTTT | | | CGGCGAGGTGATCCCCGTGATGGAACTGCTGAGCAGCATGAAAAGCCACTCTG | |
| | GATGGAAACTGGAATGGGGATCGCAGC | | | TGCCCGAGGAAATCGACATCGCCGACACTGTGTTGAACGACGATGATATCGGC | |
| | ACATATGGACTATCAATTATACTTCCA | | | GATAGCTGCCACGAGGGCTTCCTGCTGAACGCCATCAGCTCCCACCTGCAGAC | |
| | CAGACAGAACTTAGTTTCTACCTCCCA | | | ATGCGGCTGTAGCGTTGTGGTGGGCTCTAGCGCCGAAAAAGTGAACAAGATCG | |
| | CTTCATAGAGTGTGTGTTGATAGATTA | | | TGCGGACCCTTTGCCTGTTCCTGACACCTGCTGAGAGAAAGTGCAGCAGACTG | |
| | ACACATATAATCCGGAAAGGAAGAATA | | | TGTGAAGCCGAATCTAGCTTTAAGTACGAGTCCGGACTCTTCGTGCAAGGCCT | |
| | TGGATGCATAAGGAAAGACAAGAAAAT | | | GCTCAAGGACAGCACAGGCAGCTTCGTGCTGCCTTTCAGACAGGTGATGTACG | |
| | GTCCAGAAGATTATCTTAGAAGGCACA | | | CCCCTTACCCCACCACCCACATCGATGTCGACGTGAACACCGTGAAGCAGATG | |
| | GAGAGAATGGAAGATCAGGGTCAGAGT | | | CCTCCTTGCCACGAGCACATCTACAACCAGAGACGGTACATGAGAAGCGAGCT | |
| | ATTATTCCAATGCTTACTGGAGAAGTG | | | GACCGCCTTTTGGCGGGCCACCAGCGAAGAGGACATGGCTCAAGATACAATCA | |
| | ATTCCTGTAATGGAACTGCTTTCATCT | | | TCTATACCGACGAGAGCTTTACCCCTGATCTGAACATCTTTCAGGACGTGCTG | |
| | ATGAAATCACACAGTGTTCCTGAAGAA | | | CACAGAGATACCCTGGTGAAAGCCTTCCTGGATCAGGTGTTCCAGCTGAAGCC | |
| | ATAGATATAGCTGATACAGTACTCAAT | | | TGGCCTGTCTCTGCGATCTACATTCCTCGCTCAGTTCCTGCTGGTCCTGCATA | |
| | GATGATGATATTGGTGACAGCTGTCAT | | | GAAAGGCCCTGACTCTGATCAAGTACATCGAGGACGACACACAGAAGGGCAAA | |
| | GAAGGCTTTCTTCTCAATGCCATCAGC | | | AAGCCCTTCAAGTCTCTGCGGAACCTGAAAATCGACCTGGACCTGACCGCCGA | |
| | TCACACTTGCAAACCTGTGGCTGTTCC | | | GGGCGACCTGAATATCATCATGGCCCTGGCCGAGAAGATCAAACCCGGCCTGC | |
| | GTTGTAGTAGGTAGCAGTGCAGAGAAA | | | ACAGCTTCATCTTCGGAAGACCTTTCTACACCAGCGTGCAGGAGAGAGACGTG | |
| | GTAAATAAGATAGTCAGAACATTATGC | | | CTGATGACCTTCTGA (SEQ ID NO: 29) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 9 | ATGTCGACTCTTTGCCCACCGCCATCT | 40.73% | NotI | ATGAGCACCCTGTGTCCTCCACCGAGCCCTGCCGTGGCCAAGACCGAGATAGC | 56.27% |
| | CCAGCTGTTGCCAAGACAGAGATTGCT | | [GCGGCCGC] | TCTGTCCGGCAAGTCCCCACTGCTGGCCGCCACCTTCGCCTACTGGGACAACA | |
| | TTAAGTGGCAAATCACCTTTATTAGCA | | | TCCTGGGACCTAGAGTGCGGCACATCTGGGCCCCTAAGACGGAGCAGGTCCTG | |
| | GCTACTTTTGCTTACTGGGACAATATT | | | CTGAGCGACGGCGAAATAACATTCCTGGCTAATCACACCCTGAATGGCGAGAT | |
| | CTTGGTCCTAGAGTAAGGCACATTTGG | | | CCTGAGAAACGCCGAAAGCGGCGCCATCGACGTGAAGTTCTTCGTGCTGTCTG | |
| | GCTCCAAAGACAGAACAGGTACTTCTC | | | AAAAGGGAGTGATCATCGTGTCCCTGATCTTCGACGGCAACTGGAACGGCGAC | |
| | AGTGATGGAGAAATAACTTTTCTTGCC | | | CGGTCTACCTACGGCCTGAGCATCATCCTGCCCCAGACCGAACTGTCTTTTTA | |
| | AACCACACTCTAAATGGAGAAATCCTT | | | CCTGCCTCTGCACAGAGTGTGCGTGGACAGACTGACCCACATCATCCGGAAGG | |
| | CGAAATGCAGAGAGTGGTGCTATAGAT | | | GAAGAATCTGGATGCACAAGGAACGGCAGGAGAACGTGCAAAAGATCATTCTC | |
| | GTAAAGTTTTTTGTCTTGTCTGAAAAG | | | GAGGGCACCGAGAGAATGGAAGATCAGGGCCAGAGCATCATCCCCATGCTGAC | |
| | GGAGTGATTATTGTTTCATTAATCTTT | | | CGGCGAGGTGATCCCTGTGATGGAACTGCTGAGCAGCATGAAGTCCCACTCTG | |
| | GATGGAAACTGGAATGGGGATCGCAGC | | | TGCCTGAGGAAATCGACATCGCCGATACAGTGCTGAACGACGACGATATCGGC | |
| | ACATATGGACTATCAATTATACTTCCA | | | GACAGCTGCCACGAGGGCTTCCTGCTGAACGCCATCAGCTCTCACCTGCAGAC | |
| | CAGACAGAACTTAGTTTCTACCTCCCA | | | ATGCGGCTGCAGCGTGGTGGTGGGCAGCAGCGCCGAGAAGGTGAACAAGATCG | |
| | CTTCATAGAGTGTGTGTTGATAGATTA | | | TGCGGACCCTTTGCCTGTTCTTGACCCCTGCTGAGAGAAAGTGCAGCAGACTG | |
| | ACACATATAATCCGGAAAGGAAGAATA | | | TGTGAAGCCGAATCTAGCTTTAAGTACGAGTCTGGCCTCTTCGTGCAGGGACT | |
| | TGGATGCATAAGGAAAGACAAGAAAAT | | | GCTGAAGGACAGCACAGGCAGCTTCGTGCTGCCTTTTAGACAGGTGATGTACG | |
| | GTCCAGAAGATTATCTTAGAAGGCACA | | | CCCCTTACCCTACAACACACATTGACGTGGACGTTAACACCGTGAAACAGATG | |
| | GAGAGAATGGAAGATCAGGGTCAGAGT | | | CCTCCATGTCACGAGCACATCTACAACCAGAGACGGTACATGCGGAGCGAGCT | |
| | ATTATTCCAATGCTTACTGGAGAAGTG | | | GACAGCCTTTTGGCGGGCCACAAGCGAGGAAGATATGGCCCAAGACACAATCA | |
| | ATTCCTGTAATGGAACTGCTTTCATCT | | | TCTATACAGACGAGAGCTTCACCCCTGACCTGAACATCTTTCAGGACGTGCTC | |
| | ATGAAATCACACAGTGTTCCTGAAGAA | | | CATAGAGATACCCTGGTGAAGGCCTTCCTGGACCAGGTGTTCCAGCTGAAGCC | |
| | ATAGATATAGCTGATACAGTACTCAAT | | | CGGACTGAGCCTGAGATCTACATTCCTGGCCCAGTTCCTGCTGGTGCTGCACA | |
| | GATGATGATATTGGTGACAGCTGTCAT | | | GAAAGGCCCTGACACTGATCAAGTACATCGAGGATGATACACAGAAAGGCAAA | |
| | GAAGGCTTTCTTCTCAATGCCATCAGC | | | AAGCCTTTCAAGAGCCTGCGGAACCTGAAAATCGACCTGGATCTGACCGCCGA | |
| | TCACACTTGCAAACCTGTGGCTGTTCC | | | GGGAGATCTGAACATCATCATGGCCCTGGCCGAAAAGATCAAGCCCGGCCTGC | |
| | GTTGTAGTAGGTAGCAGTGCAGAGAAA | | | ACAGCTTCATCTTCGGCAGACCCTTCTACACCAGCGTGCAGGAGCGGGACGTT | |
| | GTAAATAAGATAGTCAGAACATTATGC | | | CTGATGACCTTTTGA (SEQ ID NO: 30) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 4 | ATGTCGACTCTTTGCCCACCGCCATCT | 40.73% | NotI | ATGAGCACCCTGTGCCCCCCCCCCAGCCCCGCCGTGGCCAAGACCGAGATCGC | 56.34% |
| | CCAGCTGTTGCCAAGACAGAGATTGCT | | [GCGGCCGC] | CCTGTCTGGAAAGAGCCCTCTGCTGGCCGCTACATTCGCCTACTGGGACAACA | |
| | TTAAGTGGCAAATCACCTTTATTAGCA | | | TCCTGGGCCCTAGAGTGCGGCACATCTGGGCCCCTAAGACCGAACAGGTGCTG | |
| | GCTACTTTTGCTTACTGGGACAATATT | | | CTGAGTGATGGCGAGATCACCTTCCTGGCCAACCACACCCTGAATGGAGAAAT | |
| | CTTGGTCCTAGAGTAAGGCACATTTGG | | | CCTGAGAAATGCCGAAAGCGGCGCCATCGACGTGAAGTTCTTCGTGCTGAGCG | |
| | GCTCCAAAGACAGAACAGGTACTTCTC | | | AGAAGGGCGTGATCATCGTGTCCCTGATCTTCGACGGCAACTGGAACGGCGAT | |
| | AGTGATGGAGAAATAACTTTTCTTGCC | | | AGAAGCACATACGGCCTGTCTATCATCCTGCCTCAGACAGAGCTGAGCTTCTA | |
| | AACCACACTCTAAATGGAGAAATCCTT | | | CCTGCCCCTGCACCGGGTGTGCGTGGACAGACTGACACACATTATCCGGAAAG | |
| | CGAAATGCAGAGAGTGGTGCTATAGAT | | | GCAGAATCTGGATGCACAAGGAACGGCAGGAGAACGTGCAGAAAATCATCCTG | |
| | GTAAAGTTTTTTGTCTTGTCTGAAAAG | | | GAAGGTACAGAACGGATGGAAGATCAGGGCCAGAGCATCATTCCTATGCTGAC | |
| | GGAGTGATTATTGTTTCATTAATCTTT | | | CGGCGAGGTGATCCCCGTGATGGAACTGCTATCCAGCATGAAAAGCCACTCTG | |
| | GATGGAAACTGGAATGGGGATCGCAGC | | | TGCCTGAGGAAATCGATATCGCCGACACCGTGCTGAACGACGACGACATCGGC | |
| | ACATATGGACTATCAATTATACTTCCA | | | GACTCTTGTCACGAGGGCTTCCTGCTCAATGCTATCAGCAGCCACCTGCAGAC | |
| | CAGACAGAACTTAGTTTCTACCTCCCA | | | CTGCGGCTGTTCTGTGGTCGTGGGCAGCTCCGCCGAAAAGGTGAACAAGATAG | |
| | CTTCATAGAGTGTGTGTTGATAGATTA | | | TTAGAACCCTGTGCCTGTTCCTGACCCCTGCCGAGCGGAAGTGCAGCAGACTG | |
| | ACACATATAATCCGGAAAGGAAGAATA | | | TGTGAAGCCGAGTCCAGCTTTAAGTATGAGAGCGGACTGTTCGTTCAAGGCCT | |
| | TGGATGCATAAGGAAAGACAAGAAAAT | | | GCTCAAGGACAGCACCGGCTCTTTTGTGCTCCCTTTTAGACAGGTCATGTACG | |
| | GTCCAGAAGATTATCTTAGAAGGCACA | | | CCCCTTACCCCACAACACACATCGACGTTGACGTGAACACCGTGAAGCAGATG | |
| | GAGAGAATGGAAGATCAGGGTCAGAGT | | | CCTCCTTGCCACGAGCACATCTACAACCAGAGACGGTACATGCGGAGCGAGCT | |
| | ATTATTCCAATGCTTACTGGAGAAGTG | | | GACCGCCTTTTGGCGGGCCACATCTGAAGAGGACATGGCCCAGGACACCATCA | |
| | ATTCCTGTAATGGAACTGCTTTCATCT | | | TCTACACCGACGAGAGCTTCACACCTGACCTGAATATCTTCCAAGACGTGCTG | |
| | ATGAAATCACACAGTGTTCCTGAAGAA | | | CACAGAGACACCCTGGTGAAAGCCTTCCTGGATCAGGTGTTCCAGCTGAAACC | |
| | ATAGATATAGCTGATACAGTACTCAAT | | | TGGCCTGTCCCTGCGGAGCACCTTTCTGGCCCAATTTCTGCTCGTGCTTCATA | |
| | GATGATGATATTGGTGACAGCTGTCAT | | | GAAAGGCCCTGACGCTCATCAAGTACATCGAGGATGACACACAGAAGGGCAAA | |
| | GAAGGCTTTCTTCTCAATGCCATCAGC | | | AAGCCTTTCAAGTCCCTGAGAAACCTGAAGATTGATCTGGACCTGACCGCCGA | |
| | TCACACTTGCAAACCTGTGGCTGTTCC | | | GGGAGATCTGAACATCATCATGGCCCTGGCTGAGAAGATTAAGCCCGGCCTGC | |
| | GTTGTAGTAGGTAGCAGTGCAGAGAAA | | | ACAGCTTCATCTTCGGCAGACCTTTCTACACAAGCGTGCAGGAGCGGGACGTC | |
| | GTAAATAAGATAGTCAGAACATTATGC | | | CTCATGACCTTCTGA (SEQ ID NO: 31) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 5 | ATGTCGACTCTTTGCCCACCGCCATCT | 40.73% | NotI | ATGAGCACACTCTGCCCTCCTCCTAGCCCTGCCGTGGCCAAGACCGAGATCGC | 56.34% |
| | CCAGCTGTTGCCAAGACAGAGATTGCT | | [GCGGCCGC] | CCTGAGCGGAAAGTCTCCACTGCTGGCCGCTACATTCGCCTACTGGGACAACA | |
| | TTAAGTGGCAAATCACCTTTATTAGCA | | | TACTGGGCCCTAGAGTGCGGCACATCTGGGCCCCTAAGACCGAGCAGGTCCTC | |
| | GCTACTTTTGCTTACTGGGACAATATT | | | CTGAGTGATGGAGAAATCACCTTTCTGGCTAATCACACCCTGAACGGCGAGAT | |
| | CTTGGTCCTAGAGTAAGGCACATTTGG | | | CCTGAGGAACGCCGAAAGCGGCGCCATCGACGTGAAGTTCTTCGTTCTGAGCG | |
| | GCTCCAAAGACAGAACAGGTACTTCTC | | | AGAAGGGAGTGATCATCGTGTCCCTGATCTTCGACGGCAACTGGAACGGCGAT | |
| | AGTGATGGAGAAATAACTTTTCTTGCC | | | AGATCTACATACGGCCTGAGCATCATCCTGCCTCAGACAGAGCTGTCTTTCTA | |
| | AACCACACTCTAAATGGAGAAATCCTT | | | CCTGCCTCTGCACAGAGTTTGTGTGGACCGGCTGACCCACATCATCAGAAAAG | |
| | CGAAATGCAGAGAGTGGTGCTATAGAT | | | GCCGGATCTGGATGCACAAGGAACGGCAGGAGAACGTGCAGAAAATCATCCTG | |
| | GTAAAGTTTTTTGTCTTGTCTGAAAAG | | | GAAGGCACCGAGCGGATGGAAGATCAGGGCCAGAGCATCATTCCTATGCTGAC | |
| | GGAGTGATTATTGTTTCATTAATCTTT | | | AGGCGAGGTGATCCCCGTGATGGAACTGCTGTCTTCTATGAAAAGCCACTCTG | |
| | GATGGAAACTGGAATGGGGATCGCAGC | | | TGCCCGAGGAAATCGACATCGCCGACACCGTGCTCAACGACGACGATATCGGC | |
| | ACATATGGACTATCAATTATACTTCCA | | | GACTCTTGTCACGAAGGCTTCCTGCTGAATGCCATCAGCAGCCACCTGCAGAC | |
| | CAGACAGAACTTAGTTTCTACCTCCCA | | | CTGCGGCTGTTCTGTCGTGGTGGGCTCCAGCGCCGAAAAGGTGAACAAGATAG | |
| | CTTCATAGAGTGTGTGTTGATAGATTA | | | TTAGAACCCTGTGCCTGTTCCTGACCCCTGCTGAAAGAAAGTGCAGCAGACTG | |
| | ACACATATAATCCGGAAAGGAAGAATA | | | TGCGAGGCCGAGAGCAGCTTCAAGTACGAGAGCGGCCTGTTTGTGCAAGGCCT | |
| | TGGATGCATAAGGAAAGACAAGAAAAT | | | GCTGAAGGACAGCACCGGCAGCTTCGTGCTGCCCTTCAGACAGGTGATGTACG | |
| | GTCCAGAAGATTATCTTAGAAGGCACA | | | CCCCTTATCCTACCACCCACATCGACGTGGACGTGAACACCGTGAAGCAGATG | |
| | GAGAGAATGGAAGATCAGGGTCAGAGT | | | CCCCCCTGCCACGAGCACATCTACAACCAGAGAAGATACATGAGAAGCGAGCT | |
| | ATTATTCCAATGCTTACTGGAGAAGTG | | | GACCGCCTTCTGGCGGGCCACCAGCGAGGAAGATATGGCCCAAGATACAATCA | |
| | ATTCCTGTAATGGAACTGCTTTCATCT | | | TCTACACCGACGAGAGCTTTACACCTGATCTGAACATCTTTCAGGACGTGCTG | |
| | ATGAAATCACACAGTGTTCCTGAAGAA | | | CACCGGGACACCCTGGTCAAGGCCTTTCTGGATCAGGTGTTCCAGCTGAAGCC | |
| | ATAGATATAGCTGATACAGTACTCAAT | | | TGGACTGAGCCTGAGGTCCACCTTCCTGGCCCAGTTCCTGCTGGTGCTGCATA | |
| | GATGATGATATTGGTGACAGCTGTCAT | | | GAAAGGCCCTGACCCTGATCAAGTACATCGAGGACGACACACAGAAGGGCAAG | |
| | GAAGGCTTTCTTCTCAATGCCATCAGC | | | AAGCCCTTTAAGTCCCTGCGGAACCTGAAAATCGACCTGGACCTGACAGCCGA | |
| | TCACACTTGCAAACCTGTGGCTGTTCC | | | GGGCGACCTGAACATCATCATGGCTCTGGCTGAGAAGATCAAACCCGGCCTGC | |
| | GTTGTAGTAGGTAGCAGTGCAGAGAAA | | | ACAGCTTCATCTTCGGCAGACCTTTTTACACAAGCGTGCAAGAGAGAGATGTG | |
| | GTAAATAAGATAGTCAGAACATTATGC | | | CTGATGACCTTCTGA (SEQ ID NO: 32) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 15 | ATGTCGACTCTTTGCCCACCGCCATCT | 40.73% | NotI | ATGAGCACACTGTGTCCTCCTCCGAGCCCTGCCGTGGCCAAGACCGAGATCGC | 56.41% |
| | CCAGCTGTTGCCAAGACAGAGATTGCT | | [GCGGCCGC] | CCTGAGCGGCAAGTCCCCACTGCTTGCTGCTACCTTCGCCTACTGGGACAACA | |
| | TTAAGTGGCAAATCACCTTTATTAGCA | | | TCCTGGGCCCTAGAGTGCGGCACATCTGGGCCCCTAAGACAGAGCAGGTGCTG | |
| | GCTACTTTTGCTTACTGGGACAATATT | | | CTGAGCGACGGCGAAATAACATTCCTGGCCAACCACACCCTGAACGGCGAGAT | |
| | CTTGGTCCTAGAGTAAGGCACATTTGG | | | CCTGAGAAACGCCGAGAGCGGCGCTATCGACGTGAAGTTCTTCGTTCTGTCTG | |
| | GCTCCAAAGACAGAACAGGTACTTCTC | | | AAAAGGGCGTGATCATCGTGTCCCTGATCTTCGACGGCAACTGGAACGGCGAT | |
| | AGTGATGGAGAAATAACTTTTCTTGCC | | | AGAAGCACCTACGGCCTGAGCATTATCCTGCCTCAGACAGAACTGTCTTTCTA | |
| | AACCACACTCTAAATGGAGAAATCCTT | | | CCTGCCTCTGCACAGAGTGTGCGTGGACAGACTGACACACATCATTAGAAAGG | |
| | CGAAATGCAGAGAGTGGTGCTATAGAT | | | GCAGAATCTGGATGCACAAGGAAAGACAGGAGAACGTGCAGAAGATCATCCTG | |
| | GTAAAGTTTTTTGTCTTGTCTGAAAAG | | | GAAGGCACCGAGAGAATGGAAGATCAGGGCCAGTCTATCATCCCTATGCTGAC | |
| | GGAGTGATTATTGTTTCATTAATCTTT | | | CGGCGAGGTGATCCCCGTGATGGAACTGCTGTCTAGCATGAAAAGCCACTCTG | |
| | GATGGAAACTGGAATGGGGATCGCAGC | | | TGCCCGAGGAAATCGACATCGCCGATACAGTGCTGAACGACGATGATATAGGA | |
| | ACATATGGACTATCAATTATACTTCCA | | | GATAGCTGCCATGAGGGCTTCCTGCTGAACGCCATCAGCTCCCACCTGCAGAC | |
| | CAGACAGAACTTAGTTTCTACCTCCCA | | | CTGCGGATGTAGCGTGGTCGTGGGCTCCTCCGCCGAGAAGGTGAACAAGATCG | |
| | CTTCATAGAGTGTGTGTTGATAGATTA | | | TGCGGACCCTGTGCCTGTTCCTGACACCTGCTGAACGGAAGTGCAGCAGACTG | |
| | ACACATATAATCCGGAAAGGAAGAATA | | | TGCGAGGCCGAATCTTCTTTTAAGTACGAGAGCGGACTGTTCGTGCAAGGCCT | |
| | TGGATGCATAAGGAAAGACAAGAAAAT | | | GCTGAAGGACAGCACCGGCAGCTTTGTGCTGCCATTCCGGCAGGTGATGTACG | |
| | GTCCAGAAGATTATCTTAGAAGGCACA | | | CCCCTTACCCCACCACCCACATTGACGTCGACGTGAACACCGTGAAGCAGATG | |
| | GAGAGAATGGAAGATCAGGGTCAGAGT | | | CCCCCCTGTCACGAGCACATCTACAACCAGAGGCGGTACATGAGAAGCGAGCT | |
| | ATTATTCCAATGCTTACTGGAGAAGTG | | | GACAGCCTTTTGGCGGGCCACCAGCGAGGAAGATATGGCCCAAGACACCATCA | |
| | ATTCCTGTAATGGAACTGCTTTCATCT | | | TCTACACCGACGAGAGCTTCACCCCTGATCTGAATATCTTTCAGGACGTGCTG | |
| | ATGAAATCACACAGTGTTCCTGAAGAA | | | CACAGAGATACACTGGTGAAAGCCTTCCTGGACCAGGTTTTCCAGCTGAAGCC | |
| | ATAGATATAGCTGATACAGTACTCAAT | | | TGGCCTGAGCCTGCGCAGCACCTTTCTGGCCCAGTTCCTGCTCGTGCTGCACC | |
| | GATGATGATATTGGTGACAGCTGTCAT | | | GGAAGGCCCTGACACTGATTAAGTACATCGAGGACGACACCCAGAAAGGAAAA | |
| | GAAGGCTTTCTTCTCAATGCCATCAGC | | | AAGCCCTTCAAGAGCCTGCGGAACCTGAAAATCGACCTGGACCTGACCGCCGA | |
| | TCACACTTGCAAACCTGTGGCTGTTCC | | | GGGCGACCTGAACATCATCATGGCCCTGGCCGAAAAGATCAAACCTGGACTGC | |
| | GTTGTAGTAGGTAGCAGTGCAGAGAAA | | | ATTCTTTCATCTTCGGCAGACCTTTTTACACCAGCGTGCAGGAGCGGGACGTT | |
| | GTAAATAAGATAGTCAGAACATTATGC | | | CTGATGACCTTCTGA (SEQ ID NO: 33) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 21 | ATGTCGACTCTTTGCCCACCGCCATCT | 40.73% | AscI | ATGTCTACACTCTGTCCTCCACCTAGCCCTGCTGTGGCCAAGACAGAAATCGC | 56.34% |
| | CCAGCTGTTGCCAAGACAGAGATTGCT | | [GGCGCGCC]; | CCTGAGCGGAAAAAGCCCCCTGCTGGCCGCCACCTTCGCCTACTGGGACAACA | |
| | TTAAGTGGCAAATCACCTTTATTAGCA | | NotI | TCCTGGGCCCCAGAGTCAGACACATCTGGGCCCCTAAGACCGAGCAGGTGCTG | |
| | GCTACTTTTGCTTACTGGGACAATATT | | [GCGGCCGC] | CTGAGCGACGGAGAGATCACCTTCCTGGCCAACCACACCCTGAATGGCGAGAT | |
| | CTTGGTCCTAGAGTAAGGCACATTTGG | | | CCTGCGGAACGCCGAGTCTGGCGCCATCGACGTGAAGTTCTTCGTGCTGTCTG | |
| | GCTCCAAAGACAGAACAGGTACTTCTC | | | AGAAAGGCGTGATCATTGTGTCCCTCATCTTTGACGGCAACTGGAACGGAGAT | |
| | AGTGATGGAGAAATAACTTTTCTTGCC | | | AGAAGCACCTACGGCCTGTCCATCATCCTGCCCCAGACAGAGCTGAGCTTCTA | |
| | AACCACACTCTAAATGGAGAAATCCTT | | | CCTGCCTCTGCACAGAGTGTGCGTGGACAGACTGACCCACATCATCAGAAAGG | |
| | CGAAATGCAGAGAGTGGTGCTATAGAT | | | GCAGAATCTGGATGCACAAGGAACGGCAGGAGAACGTGCAAAAAATCATCCTG | |
| | GTAAAGTTTTTTGTCTTGTCTGAAAAG | | | GAAGGCACCGAGAGAATGGAAGATCAGGGCCAGAGCATCATCCCCATGCTGAC | |
| | GGAGTGATTATTGTTTCATTAATCTTT | | | CGGCGAGGTGATCCCTGTGATGGAACTGCTGAGCAGCATGAAGTCCCATTCTG | |
| | GATGGAAACTGGAATGGGGATCGCAGC | | | TCCCCGAGGAAATCGACATCGCCGACACCGTGCTGAACGACGATGATATCGGC | |
| | ACATATGGACTATCAATTATACTTCCA | | | GATAGCTGCCACGAGGGCTTCCTGCTGAACGCCATCAGCTCTCACCTGCAGAC | |
| | CAGACAGAACTTAGTTTCTACCTCCCA | | | CTGCGGCTGCAGCGTGGTGGTCGGCTCTTCCGCCGAAAAGGTGAACAAGATCG | |
| | CTTCATAGAGTGTGTGTTGATAGATTA | | | TGCGGACCCTGTGCCTGTTCCTGACTCCTGCCGAAAGAAAGTGCTCTAGACTG | |
| | ACACATATAATCCGGAAAGGAAGAATA | | | TGTGAAGCCGAGAGCAGCTTCAAATACGAGTCCGGTCTTTTTGTGCAGGGGCT | |
| | TGGATGCATAAGGAAAGACAAGAAAAT | | | GCTGAAGGACAGCACAGGCAGCTTCGTGCTTCCATTCAGACAGGTGATGTACG | |
| | GTCCAGAAGATTATCTTAGAAGGCACA | | | CCCCTTACCCCACAACACACATTGATGTGGACGTGAACACCGTGAAGCAGATG | |
| | GAGAGAATGGAAGATCAGGGTCAGAGT | | | CCTCCTTGCCACGAGCACATCTACAACCAGCGGAGATACATGCGGAGCGAGCT | |
| | ATTATTCCAATGCTTACTGGAGAAGTG | | | GACAGCCTTCTGGCGGGCCACAAGCGAGGAAGATATGGCCCAGGACACCATCA | |
| | ATTCCTGTAATGGAACTGCTTTCATCT | | | TCTACACCGACGAGAGCTTCACCCCTGATCTGAATATCTTCCAAGACGTCCTG | |
| | ATGAAATCACACAGTGTTCCTGAAGAA | | | CACCGCGACACACTCGTGAAAGCCTTTCTCGACCAGGTTTTCCAGCTGAAACC | |
| | ATAGATATAGCTGATACAGTACTCAAT | | | TGGCCTGAGTCTGAGATCCACCTTCCTGGCTCAATTTCTGCTGGTGCTCCACC | |
| | GATGATGATATTGGTGACAGCTGTCAT | | | GGAAGGCCCTGACCCTGATCAAGTACATCGAGGACGACACCCAGAAGGGCAAG | |
| | GAAGGCTTTCTTCTCAATGCCATCAGC | | | AAGCCTTTCAAGTCTCTGAGAAACCTGAAGATCGACCTGGACCTGACAGCTGA | |
| | TCACACTTGCAAACCTGTGGCTGTTCC | | | GGGCGACCTGAATATCATCATGGCCCTTGCTGAGAAGATCAAGCCCGGCCTGC | |
| | GTTGTAGTAGGTAGCAGTGCAGAGAAA | | | ACAGCTTCATCTTCGGCAGACCTTTTTATACCAGCGTGCAGGAGAGAGATGTG | |
| | GTAAATAAGATAGTCAGAACATTATGC | | | CTGATGACCTTCTGA (SEQ ID NO: 34) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 22 | ATGTCGACTCTTTGCCCACCGCCATCT | 40.73% | AscI | ATGAGCACACTGTGCCCTCCACCTAGCCCTGCCGTGGCCAAGACCGAGATCGC | 56.20% |
| | CCAGCTGTTGCCAAGACAGAGATTGCT | | [GGCGCGCC]; | CCTTTCCGGCAAGAGCCCCCTGCTGGCCGCCACATTCGCCTACTGGGACAACA | |
| | TTAAGTGGCAAATCACCTTTATTAGCA | | NotI | TCCTGGGACCTAGAGTGCGGCACATTTGGGCCCCTAAGACCGAGCAGGTCCTG | |
| | GCTACTTTTGCTTACTGGGACAATATT | | [GCGGCCGC] | CTGAGTGATGGCGAAATCACCTTCCTGGCCAACCACACCCTGAACGGCGAGAT | |
| | CTTGGTCCTAGAGTAAGGCACATTTGG | | | CCTGCGGAACGCCGAGAGCGGTGCTATCGATGTGAAGTTCTTCGTGCTGAGCG | |
| | GCTCCAAAGACAGAACAGGTACTTCTC | | | AGAAGGGCGTGATCATCGTGTCCCTCATCTTCGACGGCAACTGGAACGGCGAC | |
| | AGTGATGGAGAAATAACTTTTCTTGCC | | | AGATCTACATACGGCCTGTCTATCATCCTGCCTCAGACCGAACTGTCCTTCTA | |
| | AACCACACTCTAAATGGAGAAATCCTT | | | CCTGCCTCTGCACCGGGTGTGCGTGGACCGGCTGACTCACATCATCAGAAAGG | |
| | CGAAATGCAGAGAGTGGTGCTATAGAT | | | GCAGAATCTGGATGCACAAGGAACGGCAGGAGAACGTGCAAAAGATCATTCTG | |
| | GTAAAGTTTTTTGTCTTGTCTGAAAAG | | | GAAGGTACAGAGAGAATGGAAGATCAGGGCCAGAGCATTATCCCTATGCTGAC | |
| | GGAGTGATTATTGTTTCATTAATCTTT | | | AGGCGAGGTGATCCCCGTGATGGAACTGTTGTCCTCCATGAAGTCCCACTCTG | |
| | GATGGAAACTGGAATGGGGATCGCAGC | | | TTCCTGAGGAAATCGACATCGCCGACACAGTGCTGAACGACGACGATATCGGC | |
| | ACATATGGACTATCAATTATACTTCCA | | | GACAGCTGCCACGAGGGCTTCCTGCTGAACGCCATCAGCAGCCACCTGCAGAC | |
| | CAGACAGAACTTAGTTTCTACCTCCCA | | | CTGTGGCTGTAGCGTGGTCGTGGGCTCTAGCGCCGAAAAGGTGAACAAGATCG | |
| | CTTCATAGAGTGTGTGTTGATAGATTA | | | TGCGGACCCTGTGTCTGTTCCTGACACCTGCTGAGAGAAAGTGCAGCAGACTG | |
| | ACACATATAATCCGGAAAGGAAGAATA | | | TGCGAGGCCGAGTCTAGCTTTAAGTACGAGAGCGGCCTGTTCGTGCAGGGCCT | |
| | TGGATGCATAAGGAAAGACAAGAAAAT | | | CCTGAAGGACAGCACCGGCAGCTTTGTGCTGCCCTTCAGACAGGTGATGTACG | |
| | GTCCAGAAGATTATCTTAGAAGGCACA | | | CCCCTTACCCCACCACCCACATCGACGTGGACGTGAACACCGTGAAGCAGATG | |
| | GAGAGAATGGAAGATCAGGGTCAGAGT | | | CCTCCGTGCCATGAGCACATCTACAACCAGAGAAGATACATGAGAAGCGAGCT | |
| | ATTATTCCAATGCTTACTGGAGAAGTG | | | GACCGCTTTCTGGCGGGCCACCTCTGAGGAAGATATGGCCCAGGACACCATCA | |
| | ATTCCTGTAATGGAACTGCTTTCATCT | | | TCTATACAGACGAGAGCTTCACCCCTGATCTGAATATTTTCCAAGATGTGCTG | |
| | ATGAAATCACACAGTGTTCCTGAAGAA | | | CACAGAGATACACTTGTGAAAGCCTTCCTCGACCAGGTGTTCCAGCTGAAGCC | |
| | ATAGATATAGCTGATACAGTACTCAAT | | | TGGCCTGTCTCTGCGGAGCACCTTTCTGGCACAGTTCCTGCTGGTGCTGCATA | |
| | GATGATGATATTGGTGACAGCTGTCAT | | | GAAAGGCCCTGACCTTGATCAAGTACATCGAGGATGACACCCAGAAAGGAAAG | |
| | GAAGGCTTTCTTCTCAATGCCATCAGC | | | AAACCTTTCAAGAGCCTGAGAAACCTGAAAATCGACCTGGACCTGACGGCCGA | |
| | TCACACTTGCAAACCTGTGGCTGTTCC | | | AGGCGATCTGAATATCATCATGGCCCTGGCCGAGAAGATCAAGCCCGGCCTGC | |
| | GTTGTAGTAGGTAGCAGTGCAGAGAAA | | | ACAGCTTCATCTTTGGAAGACCTTTTTACACCAGCGTGCAGGAGCGGGACGTG | |
| | GTAAATAAGATAGTCAGAACATTATGC | | | CTGATGACATTTTGA (SEQ ID NO: 190) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 23 | ATGTCGACTCTTTGCCCACCGCCATCT | 40.73% | AscI | ATGAGCACCCTGTGTCCTCCACCTAGCCCCGCCGTGGCCAAGACCGAGATCGC | 55.93% |
| | CCAGCTGTTGCCAAGACAGAGATTGCT | | [GGCGCGCC]; | CCTGTCTGGAAAGTCCCCTCTGCTGGCCGCTACATTCGCCTACTGGGACAACA | |
| | TTAAGTGGCAAATCACCTTTATTAGCA | | NotI | TCCTGGGACCTAGAGTGCGGCACATCTGGGCCCCTAAGACCGAGCAGGTGCTC | |
| | GCTACTTTTGCTTACTGGGACAATATT | | [GCGGCCGC] | CTGAGTGATGGCGAGATAACATTTCTGGCCAACCACACCCTCAACGGCGAGAT | |
| | CTTGGTCCTAGAGTAAGGCACATTTGG | | | CCTGAGAAACGCCGAAAGCGGCGCCATCGACGTGAAGTTCTTCGTGCTGTCTG | |
| | GCTCCAAAGACAGAACAGGTACTTCTC | | | AAAAGGGCGTGATCATCGTGTCCCTGATCTTCGACGGCAACTGGAACGGCGAC | |
| | AGTGATGGAGAAATAACTTTTCTTGCC | | | AGAAGCACGTACGGCCTGTCCATCATCCTGCCCCAGACCGAGCTGTCTTTCTA | |
| | AACCACACTCTAAATGGAGAAATCCTT | | | CCTGCCTCTGCACCGGGTGTGCGTGGATAGACTGACCCACATTATTAGAAAGG | |
| | CGAAATGCAGAGAGTGGTGCTATAGAT | | | GCAGAATCTGGATGCACAAGGAACGCCAGGAGAACGTGCAGAAGATCATCCTG | |
| | GTAAAGTTTTTTGTCTTGTCTGAAAAG | | | GAAGGTACAGAGCGGATGGAAGATCAGGGCCAGAGCATCATCCCCATGCTGAC | |
| | GGAGTGATTATTGTTTCATTAATCTTT | | | CGGCGAAGTGATCCCTGTGATGGAACTGCTGAGTTCTATGAAAAGCCACAGCG | |
| | GATGGAAACTGGAATGGGGATCGCAGC | | | TGCCGGAAGAGATCGATATCGCCGACACCGTCCTTAACGACGACGACATAGGA | |
| | ACATATGGACTATCAATTATACTTCCA | | | GATAGCTGCCACGAGGGCTTCCTTCTGAACGCCATCAGCTCTCACCTGCAGAC | |
| | CAGACAGAACTTAGTTTCTACCTCCCA | | | ATGCGGCTGCAGCGTCGTGGTCGGCTCTAGCGCCGAAAAAGTGAACAAGATCG | |
| | CTTCATAGAGTGTGTGTTGATAGATTA | | | TGCGGACCCTGTGCCTGTTCCTGACACCTGCCGAGAGAAAGTGCTCTAGACTG | |
| | ACACATATAATCCGGAAAGGAAGAATA | | | TGCGAGGCCGAGTCCAGCTTCAAGTACGAGAGCGGCCTGTTTGTTCAAGGACT | |
| | TGGATGCATAAGGAAAGACAAGAAAAT | | | GCTGAAGGACAGCACCGGCAGCTTTGTGCTCCCTTTTAGACAGGTGATGTACG | |
| | GTCCAGAAGATTATCTTAGAAGGCACA | | | CCCCTTACCCCACCACCCACATCGACGTTGACGTGAATACCGTGAAACAGATG | |
| | GAGAGAATGGAAGATCAGGGTCAGAGT | | | CCTCCTTGTCACGAGCACATCTACAACCAGAGAAGATACATGAGATCTGAGCT | |
| | ATTATTCCAATGCTTACTGGAGAAGTG | | | GACCGCCTTCTGGCGGGCCACCAGCGAGGAAGATATGGCCCAGGACACCATCA | |
| | ATTCCTGTAATGGAACTGCTTTCATCT | | | TCTACACCGACGAGAGCTTCACCCCTGATCTGAACATCTTTCAGGATGTCCTG | |
| | ATGAAATCACACAGTGTTCCTGAAGAA | | | CACCGCGACACCCTGGTCAAAGCCTTTCTGGACCAGGTGTTCCAGCTGAAACC | |
| | ATAGATATAGCTGATACAGTACTCAAT | | | CGGACTGTCTCTGCGGAGCACCTTCTTGGCTCAATTTCTCCTGGTGCTGCACA | |
| | GATGATGATATTGGTGACAGCTGTCAT | | | GAAAGGCCCTGACACTGATCAAGTACATCGAGGATGATACACAGAAAGGCAAA | |
| | GAAGGCTTTCTTCTCAATGCCATCAGC | | | AAGCCCTTCAAGAGCCTGAGAAATCTGAAGATCGACCTGGACCTGACAGCCGA | |
| | TCACACTTGCAAACCTGTGGCTGTTCC | | | GGGCGATCTGAACATCATCATGGCCCTGGCTGAGAAGATTAAGCCTGGCCTCC | |
| | GTTGTAGTAGGTAGCAGTGCAGAGAAA | | | ATTCTTTCATCTTCGGCAGACCTTTCTACACCAGCGTGCAGGAGCGGGACGTG | |
| | GTAAATAAGATAGTCAGAACATTATGC | | | CTGATGACATTCTGA (SEQ ID NO: 35) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 24 | ATGTCGACTCTTTGCCCACCGCCATCT | 40.73% | AscI | ATGAGCACCCTGTGTCCTCCTCCATCTCCAGCCGTGGCCAAGACCGAGATCGC | 56.13% |
| | CCAGCTGTTGCCAAGACAGAGATTGCT | | [GGCGCGCC]; | CCTGTCCGGCAAGAGCCCTCTGCTGGCCGCTACATTCGCCTACTGGGACAACA | |
| | TTAAGTGGCAAATCACCTTTATTAGCA | | NotI | TCCTGGGACCTAGAGTGCGGCACATCTGGGCCCCTAAGACAGAGCAGGTGCTG | |
| | GCTACTTTTGCTTACTGGGACAATATT | | [GCGGCCGC] | CTGAGTGATGGCGAGATCACCTTCCTGGCCAACCACACCCTGAATGGAGAAAT | |
| | CTTGGTCCTAGAGTAAGGCACATTTGG | | | CCTGAGAAACGCCGAGAGTGGCGCCATCGATGTGAAGTTCTTCGTGCTGTCTG | |
| | GCTCCAAAGACAGAACAGGTACTTCTC | | | AAAAGGGCGTGATCATCGTCAGCCTGATCTTCGACGGCAACTGGAACGGCGAC | |
| | AGTGATGGAGAAATAACTTTTCTTGCC | | | AGAAGCACATACGGCCTGAGCATCATCCTGCCCCAGACAGAGCTGTCTTTTTA | |
| | AACCACACTCTAAATGGAGAAATCCTT | | | CCTGCCTCTGCACAGAGTGTGCGTGGACCGGCTGACCCACATCATTAGAAAGG | |
| | CGAAATGCAGAGAGTGGTGCTATAGAT | | | GCAGAATCTGGATGCACAAGGAAAGACAGGAGAACGTGCAGAAGATCATCCTG | |
| | GTAAAGTTTTTTGTCTTGTCTGAAAAG | | | GAAGGTACAGAGAGAATGGAAGATCAGGGACAGAGCATCATCCCCATGCTGAC | |
| | GGAGTGATTATTGTTTCATTAATCTTT | | | CGGCGAAGTGATCCCTGTGATGGAACTGCTGAGCAGCATGAAAAGCCATTCTG | |
| | GATGGAAACTGGAATGGGGATCGCAGC | | | TGCCCGAGGAAATCGACATCGCCGACACAGTGCTGAACGACGACGATATCGGC | |
| | ACATATGGACTATCAATTATACTTCCA | | | GATAGCTGCCACGAGGGATTCCTGCTTAATGCCATCAGCAGCCACCTGCAGAC | |
| | CAGACAGAACTTAGTTTCTACCTCCCA | | | CTGTGGCTGTAGCGTGGTCGTGGGCAGCTCCGCCGAGAAGGTGAACAAGATCG | |
| | CTTCATAGAGTGTGTGTTGATAGATTA | | | TGAGGACCCTCTGCCTGTTCCTGACACCTGCTGAAAGAAAGTGCAGCAGACTG | |
| | ACACATATAATCCGGAAAGGAAGAATA | | | TGCGAGGCCGAGTCCAGCTTCAAGTACGAGAGCGGCCTCTTCGTGCAGGGCCT | |
| | TGGATGCATAAGGAAAGACAAGAAAAT | | | GCTGAAGGACAGCACCGGCTCCTTCGTGCTGCCTTTTAGACAGGTGATGTACG | |
| | GTCCAGAAGATTATCTTAGAAGGCACA | | | CCCCTTACCCCACCACCCACATTGACGTGGACGTGAACACCGTGAAGCAGATG | |
| | GAGAGAATGGAAGATCAGGGTCAGAGT | | | CCTCCGTGCCACGAGCACATCTACAACCAGCGCAGATACATGCGGAGCGAGCT | |
| | ATTATTCCAATGCTTACTGGAGAAGTG | | | GACCGCCTTCTGGCGGGCCACATCTGAGGAAGATATGGCTCAAGATACCATCA | |
| | ATTCCTGTAATGGAACTGCTTTCATCT | | | TCTACACCGACGAGAGCTTCACCCCTGATCTGAACATCTTCCAGGACGTGCTG | |
| | ATGAAATCACACAGTGTTCCTGAAGAA | | | CATAGAGATACCCTGGTGAAAGCTTTCCTTGATCAGGTTTTCCAACTGAAGCC | |
| | ATAGATATAGCTGATACAGTACTCAAT | | | TGGCCTGAGCCTGAGAAGCACCTTCCTGGCTCAGTTCCTGCTGGTGCTTCACC | |
| | GATGATGATATTGGTGACAGCTGTCAT | | | GGAAGGCCCTAACCCTGATCAAGTACATCGAGGATGACACCCAGAAAGGCAAA | |
| | GAAGGCTTTCTTCTCAATGCCATCAGC | | | AAGCCTTTTAAGTCCCTGCGGAACCTGAAAATCGACCTGGACCTCACAGCCGA | |
| | TCACACTTGCAAACCTGTGGCTGTTCC | | | GGGAGATCTGAACATCATCATGGCCCTGGCCGAAAAGATAAAGCCCGGCCTGC | |
| | GTTGTAGTAGGTAGCAGTGCAGAGAAA | | | ACAGCTTCATCTTTGGCAGACCTTTCTACACAAGCGTGCAGGAGCGGGACGTG | |
| | GTAAATAAGATAGTCAGAACATTATGC | | | CTGATGACCTTCTGA (SEQ ID NO: 36) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 25 | ATGTCGACTCTTTGCCCACCGCCATCT | 40.73% | AscI | ATGAGCACCCTCTGTCCTCCACCTAGCCCTGCTGTGGCCAAGACCGAAATTGC | 56.06% |
| | CCAGCTGTTGCCAAGACAGAGATTGCT | | [GGCGCGCC]; | CCTGAGCGGAAAGTCTCCTCTGTTGGCTGCTACATTCGCCTACTGGGACAACA | |
| | TTAAGTGGCAAATCACCTTTATTAGCA | | Not I | TCCTGGGCCCTAGAGTGCGGCACATCTGGGCCCCTAAGACAGAGCAGGTGCTG | |
| | GCTACTTTTGCTTACTGGGACAATATT | | [GCGGCCGC] | CTGAGTGATGGCGAAATCACCTTCCTGGCCAACCACACCCTGAACGGCGAGAT | |
| | CTTGGTCCTAGAGTAAGGCACATTTGG | | | CCTGAGAAACGCCGAAAGCGGCGCCATCGACGTGAAGTTCTTCGTGCTGTCTG | |
| | GCTCCAAAGACAGAACAGGTACTTCTC | | | AAAAGGGTGTTATCATTGTGTCCCTGATCTTTGACGGCAACTGGAACGGCGAC | |
| | AGTGATGGAGAAATAACTTTTCTTGCC | | | AGATCTACATACGGCCTGTCCATCATCCTGCCTCAGACCGAGCTGTCTTTCTA | |
| | AACCACACTCTAAATGGAGAAATCCTT | | | CCTGCCTCTGCACAGAGTGTGCGTGGACCGGCTGACTCATATCATCAGAAAGG | |
| | CGAAATGCAGAGAGTGGTGCTATAGAT | | | GAAGAATCTGGATGCACAAGGAAAGACAGGAGAACGTGCAGAAGATCATCCTG | |
| | GTAAAGTTTTTTGTCTTGTCTGAAAAG | | | GAAGGTACAGAGAGAATGGAAGATCAGGGCCAGAGCATCATCCCCATGCTGAC | |
| | GGAGTGATTATTGTTTCATTAATCTTT | | | AGGCGAGGTGATCCCTGTGATGGAACTGCTGAGCAGCATGAAGTCCCACAGCG | |
| | GATGGAAACTGGAATGGGGATCGCAGC | | | TCCCCGAGGAAATCGACATCGCCGACACAGTGCTGAACGACGACGATATCGGC | |
| | ACATATGGACTATCAATTATACTTCCA | | | GATTCATGCCACGAGGGCTTCCTGCTGAATGCAATCAGCAGCCACCTGCAGAC | |
| | CAGACAGAACTTAGTTTCTACCTCCCA | | | CTGCGGCTGTTCTGTGGTGGTGGGCAGCAGCGCCGAAAAAGTGAACAAGATCG | |
| | CTTCATAGAGTGTGTGTTGATAGATTA | | | TGCGCACCCTGTGCCTGTTTTTGACCCCTGCCGAGCGGAAGTGCAGCAGACTG | |
| | ACACATATAATCCGGAAAGGAAGAATA | | | TGTGAAGCCGAGAGCTCTTTCAAGTACGAGAGCGGCCTGTTCGTTCAAGGCCT | |
| | TGGATGCATAAGGAAAGACAAGAAAAT | | | GCTGAAGGACAGCACCGGCAGCTTTGTGCTGCCCTTCCGGCAGGTGATGTACG | |
| | GTCCAGAAGATTATCTTAGAAGGCACA | | | CCCCTTACCCCACCACCCACATCGACGTCGACGTGAACACCGTGAAGCAGATG | |
| | GAGAGAATGGAAGATCAGGGTCAGAGT | | | CCTCCGTGCCACGAGCACATCTACAACCAGCGGAGATACATGCGGTCCGAGCT | |
| | ATTATTCCAATGCTTACTGGAGAAGTG | | | GACAGCCTTCTGGCGGGCCACCAGCGAAGAGGACATGGCCCAGGACACCATCA | |
| | ATTCCTGTAATGGAACTGCTTTCATCT | | | TCTACACTGATGAGTCCTTCACACCTGATCTGAATATCTTCCAAGACGTGCTT | |
| | ATGAAATCACACAGTGTTCCTGAAGAA | | | CACAGAGACACCCTGGTGAAAGCTTTTCTCGACCAGGTTTTCCAGCTGAAGCC | |
| | ATAGATATAGCTGATACAGTACTCAAT | | | CGGCCTGAGCCTGAGATCTACCTTCCTGGCTCAATTTCTGCTCGTGCTGCACA | |
| | GATGATGATATTGGTGACAGCTGTCAT | | | GAAAGGCCCTGACGCTGATCAAGTATATCGAGGACGACACGCAGAAAGGCAAG | |
| | GAAGGCTTTCTTCTCAATGCCATCAGC | | | AAACCCTTCAAAAGCCTGCGGAACCTGAAAATTGACCTGGACCTGACCGCCGA | |
| | TCACACTTGCAAACCTGTGGCTGTTCC | | | GGGCGACCTGAACATCATCATGGCCCTGGCCGAGAAGATCAAGCCTGGACTGC | |
| | GTTGTAGTAGGTAGCAGTGCAGAGAAA | | | ATAGCTTCATCTTCGGCAGACCTTTTTACACCTCTGTGCAGGAGCGGGACGTG | |
| | GTAAATAAGATAGTCAGAACATTATGC | | | CTCATGACCTTTTGA (SEQ ID NO: 37) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 26 | ATGTCGACTCTTTGCCCACCGCCATCT | 40.73% | AscI | ATGAGCACCCTGTGTCCTCCTCCAAGCCCTGCCGTGGCCAAGACAGAGATCGC | 56.48% |
| | CCAGCTGTTGCCAAGACAGAGATTGCT | | [GGCGCGCC]; | CCTTAGCGGAAAGTCCCCTCTGCTGGCCGCCACATTTGCCTACTGGGACAACA | |
| | TTAAGTGGCAAATCACCTTTATTAGCA | | NotI | TCCTGGGACCTAGAGTGCGGCACATTTGGGCCCCAAAGACCGAGCAGGTGCTG | |
| | GCTACTTTTGCTTACTGGGACAATATT | | [GCGGCCGC] | CTGAGCGACGGCGAAATCACCTTCCTGGCTAATCACACACTGAACGGCGAGAT | |
| | CTTGGTCCTAGAGTAAGGCACATTTGG | | | CCTGAGGAACGCCGAAAGCGGCGCCATCGACGTGAAGTTCTTCGTCCTGAGCG | |
| | GCTCCAAAGACAGAACAGGTACTTCTC | | | AGAAGGGCGTGATCATTGTGTCCCTGATCTTCGACGGCAACTGGAACGGCGAC | |
| | AGTGATGGAGAAATAACTTTTCTTGCC | | | CGCTCCACATACGGCCTGTCTATCATCCTGCCCCAGACCGAGCTGTCTTTTTA | |
| | AACCACACTCTAAATGGAGAAATCCTT | | | CCTGCCTCTGCACAGAGTGTGCGTGGACAGACTGACCCACATCATCCGGAAGG | |
| | CGAAATGCAGAGAGTGGTGCTATAGAT | | | GCAGAATCTGGATGCACAAGGAACGGCAGGAGAACGTGCAGAAAATCATCCTG | |
| | GTAAAGTTTTTTGTCTTGTCTGAAAAG | | | GAAGGAACAGAGCGGATGGAAGATCAGGGCCAGAGCATCATACCCATGCTGAC | |
| | GGAGTGATTATTGTTTCATTAATCTTT | | | TGGCGAGGTGATCCCTGTGATGGAACTGCTGTCAAGCATGAAAAGCCACTCTG | |
| | GATGGAAACTGGAATGGGGATCGCAGC | | | TCCCCGAGGAAATCGACATCGCTGATACCGTGCTCAACGACGACGATATCGGC | |
| | ACATATGGACTATCAATTATACTTCCA | | | GATAGCTGCCACGAGGGCTTCCTGCTGAACGCCATCAGCAGCCACCTGCAGAC | |
| | CAGACAGAACTTAGTTTCTACCTCCCA | | | ATGCGGCTGCAGCGTCGTGGTGGGCTCTAGCGCCGAAAAGGTGAACAAGATCG | |
| | CTTCATAGAGTGTGTGTTGATAGATTA | | | TGCGGACCCTGTGTCTGTTCTTGACCCCTGCTGAAAGAAAGTGCAGCAGACTG | |
| | ACACATATAATCCGGAAAGGAAGAATA | | | TGCGAGGCCGAGAGCAGCTTCAAGTACGAGTCTGGCCTGTTTGTGCAGGGCCT | |
| | TGGATGCATAAGGAAAGACAAGAAAAT | | | GCTGAAAGACAGCACAGGCAGCTTCGTGCTGCCCTTCAGACAGGTGATGTACG | |
| | GTCCAGAAGATTATCTTAGAAGGCACA | | | CCCCTTACCCTACCACCCACATTGACGTGGACGTGAACACCGTGAAGCAGATG | |
| | GAGAGAATGGAAGATCAGGGTCAGAGT | | | CCTCCGTGCCACGAGCACATCTACAACCAGCGTAGATACATGAGATCCGAGCT | |
| | ATTATTCCAATGCTTACTGGAGAAGTG | | | GACAGCTTTCTGGCGGGCCACCTCTGAAGAGGATATGGCCCAGGACACCATCA | |
| | ATTCCTGTAATGGAACTGCTTTCATCT | | | TCTATACCGACGAGAGCTTCACCCCTGATCTGAATATCTTCCAAGACGTGCTG | |
| | ATGAAATCACACAGTGTTCCTGAAGAA | | | CATAGAGACACCCTGGTGAAAGCCTTCCTGGATCAAGTGTTCCAGCTGAAGCC | |
| | ATAGATATAGCTGATACAGTACTCAAT | | | TGGACTGAGCCTGCGGAGCACCTTCCTGGCCCAGTTCCTGCTCGTGCTTCATA | |
| | GATGATGATATTGGTGACAGCTGTCAT | | | GAAAGGCCCTGACACTGATCAAGTACATCGAGGACGACACACAGAAGGGCAAA | |
| | GAAGGCTTTCTTCTCAATGCCATCAGC | | | AAGCCCTTCAAGAGCCTGAGAAACCTGAAGATCGACCTGGACCTGACCGCCGA | |
| | TCACACTTGCAAACCTGTGGCTGTTCC | | | GGGCGATCTGAACATCATCATGGCTCTGGCCGAGAAGATCAAGCCCGGCCTGC | |
| | GTTGTAGTAGGTAGCAGTGCAGAGAAA | | | ACAGCTTTATCTTTGGCAGACCTTTCTACACCAGCGTGCAAGAGAGAGATGTG | |
| | GTAAATAAGATAGTCAGAACATTATGC | | | CTGATGACCTTTTGA (SEQ ID NO: 38) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 27 | ATGTCGACTCTTTGCCCACCGCCATCT | 40.73% | AscI | ATGTCTACCCTGTGTCCTCCTCCAAGCCCCGCCGTGGCCAAGACTGAGATCGC | 56.13% |
| | CCAGCTGTTGCCAAGACAGAGATTGCT | | [GGCGCGCC]; | CCTGAGCGGCAAATCTCCTCTGCTCGCTGCTACCTTCGCCTACTGGGACAACA | |
| | TTAAGTGGCAAATCACCTTTATTAGCA | | NotI | TCCTGGGACCTAGAGTGCGGCACATCTGGGCCCCTAAGACCGAGCAGGTCCTG | |
| | GCTACTTTTGCTTACTGGGACAATATT | | [GCGGCCGC] | CTGAGCGACGGAGAGATAACATTTCTGGCCAACCACACACTGAACGGCGAGAT | |
| | CTTGGTCCTAGAGTAAGGCACATTTGG | | | CCTCAGAAATGCCGAGAGCGGCGCCATCGACGTGAAGTTCTTCGTGCTGTCTG | |
| | GCTCCAAAGACAGAACAGGTACTTCTC | | | AGAAGGGCGTGATCATTGTGTCCCTGATCTTCGACGGCAACTGGAACGGCGAC | |
| | AGTGATGGAGAAATAACTTTTCTTGCC | | | AGAAGCACCTACGGCCTGAGCATCATCCTGCCTCAGACAGAGCTGTCCTTTTA | |
| | AACCACACTCTAAATGGAGAAATCCTT | | | CCTGCCACTGCACCGGGTGTGCGTGGATAGACTGACACACATCATTAGAAAGG | |
| | CGAAATGCAGAGAGTGGTGCTATAGAT | | | GCAGAATCTGGATGCACAAGGAAAGACAGGAGAACGTGCAGAAAATCATCCTG | |
| | GTAAAGTTTTTTGTCTTGTCTGAAAAG | | | GAAGGTACAGAGCGGATGGAAGATCAGGGCCAGAGCATCATCCCTATGCTGAC | |
| | GGAGTGATTATTGTTTCATTAATCTTT | | | CGGCGAGGTGATCCCCGTTATGGAACTCCTGTCTTCTATGAAAAGCCACAGCG | |
| | GATGGAAACTGGAATGGGGATCGCAGC | | | TCCCCGAGGAAATCGACATCGCAGATACAGTGCTGAACGACGACGATATAGGA | |
| | ACATATGGACTATCAATTATACTTCCA | | | GATAGCTGTCACGAGGGCTTCCTGTTAAACGCCATCAGCAGCCACCTGCAGAC | |
| | CAGACAGAACTTAGTTTCTACCTCCCA | | | CTGTGGCTGCAGCGTGGTGGTCGGCTCTAGCGCCGAAAAGGTGAACAAGATCG | |
| | CTTCATAGAGTGTGTGTTGATAGATTA | | | TGCGGACCCTGTGCCTGTTCCTGACACCTGCTGAACGGAAGTGCAGCAGACTG | |
| | ACACATATAATCCGGAAAGGAAGAATA | | | TGCGAGGCCGAGAGCAGTTTTAAGTACGAGTCCGGCCTGTTCGTGCAAGGCCT | |
| | TGGATGCATAAGGAAAGACAAGAAAAT | | | GCTGAAGGACTCTACAGGCAGCTTCGTGCTGCCTTTCAGACAGGTGATGTACG | |
| | GTCCAGAAGATTATCTTAGAAGGCACA | | | CCCCTTACCCCACCACCCACATCGACGTGGACGTGAACACCGTGAAGCAGATG | |
| | GAGAGAATGGAAGATCAGGGTCAGAGT | | | CCTCCGTGCCACGAGCACATCTACAACCAGCGGAGATACATGCGGAGCGAGCT | |
| | ATTATTCCAATGCTTACTGGAGAAGTG | | | GACCGCTTTCTGGCGGGCCACCAGCGAAGAGGACATGGCTCAGGACACCATCA | |
| | ATTCCTGTAATGGAACTGCTTTCATCT | | | TCTATACAGACGAGAGCTTCACCCCTGACCTGAATATCTTTCAAGACGTGCTG | |
| | ATGAAATCACACAGTGTTCCTGAAGAA | | | CACAGAGATACCCTCGTGAAAGCCTTCCTGGACCAGGTGTTCCAGCTGAAACC | |
| | ATAGATATAGCTGATACAGTACTCAAT | | | TGGACTGTCACTGAGAAGCACCTTTCTGGCCCAGTTCCTGCTGGTCCTGCACA | |
| | GATGATGATATTGGTGACAGCTGTCAT | | | GAAAGGCCCTGACCCTTATCAAGTACATCGAGGATGACACCCAGAAGGGCAAG | |
| | GAAGGCTTTCTTCTCAATGCCATCAGC | | | AAGCCCTTCAAGAGCCTGAGAAACCTGAAGATCGACCTGGATCTGACAGCCGA | |
| | TCACACTTGCAAACCTGTGGCTGTTCC | | | AGGCGACCTGAACATCATCATGGCCCTGGCCGAAAAGATTAAGCCTGGCCTGC | |
| | GTTGTAGTAGGTAGCAGTGCAGAGAAA | | | ATTCTTTCATCTTCGGCCGCCCCTTCTACACCAGCGTGCAGGAGAGAGATGTG | |
| | GTAAATAAGATAGTCAGAACATTATGC | | | CTGATGACCTTCTGA (SEQ ID NO: 39) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 28 | ATGTCGACTCTTTGCCCACCGCCATCT | 40.73% | AscI | ATGAGCACCCTGTGTCCTCCTCCTAGCCCTGCCGTGGCAAAGACCGAGATCGC | 55.93% |
| | CCAGCTGTTGCCAAGACAGAGATTGCT | | [GGCGCGCC]; | CCTGAGCGGGAAGTCACCCCTGCTGGCCGCTACATTTGCCTACTGGGACAACA | |
| | TTAAGTGGCAAATCACCTTTATTAGCA | | NotI | TCCTGGGCCCTAGAGTGCGGCACATCTGGGCCCCTAAGACCGAGCAGGTGCTG | |
| | GCTACTTTTGCTTACTGGGACAATATT | | [GCGGCCGC] | CTCAGTGATGGCGAGATAACATTCCTCGCCAACCACACACTGAATGGCGAAAT | |
| | CTTGGTCCTAGAGTAAGGCACATTTGG | | | CCTTAGAAATGCCGAGAGCGGTGCTATCGACGTAAAGTTCTTCGTGCTGTCTG | |
| | GCTCCAAAGACAGAACAGGTACTTCTC | | | AAAAGGGCGTGATCATCGTGTCCCTGATCTTCGACGGCAACTGGAACGGCGAT | |
| | AGTGATGGAGAAATAACTTTTCTTGCC | | | AGAAGCACCTACGGCCTGAGCATCATCCTGCCTCAGACAGAGCTGAGCTTCTA | |
| | AACCACACTCTAAATGGAGAAATCCTT | | | TCTGCCTCTGCACAGGGTGTGCGTGGACAGACTGACTCACATTATTAGAAAAG | |
| | CGAAATGCAGAGAGTGGTGCTATAGAT | | | GCAGAATCTGGATGCACAAGGAAAGACAGGAGAACGTGCAAAAGATCATCCTG | |
| | GTAAAGTTTTTTGTCTTGTCTGAAAAG | | | GAAGGCACCGAGAGAATGGAAGATCAGGGCCAGAGCATCATCCCTATGCTGAC | |
| | GGAGTGATTATTGTTTCATTAATCTTT | | | CGGCGAGGTGATCCCCGTGATGGAACTGCTGAGTTCTATGAAGAGTCACTCTG | |
| | GATGGAAACTGGAATGGGGATCGCAGC | | | TGCCCGAGGAAATCGACATCGCCGACACAGTGCTGAACGACGACGATATCGGC | |
| | ACATATGGACTATCAATTATACTTCCA | | | GACTCCTGCCACGAGGGCTTCCTGCTGAACGCCATCAGCAGCCACCTGCAGAC | |
| | CAGACAGAACTTAGTTTCTACCTCCCA | | | CTGCGGCTGCAGCGTGGTGGTCGGCAGCTCCGCCGAAAAGGTGAACAAGATCG | |
| | CTTCATAGAGTGTGTGTTGATAGATTA | | | TGCGGACCCTGTGCCTGTTCCTGACGCCCGCCGAAAGAAAGTGCAGTAGACTG | |
| | ACACATATAATCCGGAAAGGAAGAATA | | | TGCGAGGCCGAAAGCTCTTTCAAGTACGAGAGCGGCCTGTTTGTGCAGGGCCT | |
| | TGGATGCATAAGGAAAGACAAGAAAAT | | | GCTCAAGGACAGCACTGGATCTTTCGTGCTCCCCTTCAGACAGGTGATGTACG | |
| | GTCCAGAAGATTATCTTAGAAGGCACA | | | CCCCTTACCCTACAACACACATCGATGTGGACGTGAACACCGTGAAGCAGATG | |
| | GAGAGAATGGAAGATCAGGGTCAGAGT | | | CCTCCATGTCACGAGCACATCTACAACCAGCGTAGATACATGAGAAGCGAGCT | |
| | ATTATTCCAATGCTTACTGGAGAAGTG | | | GACAGCCTTTTGGCGGGCCACAAGCGAGGAAGATATGGCCCAGGACACCATCA | |
| | ATTCCTGTAATGGAACTGCTTTCATCT | | | TCTACACCGACGAGAGCTTCACCCCTGACCTGAATATCTTTCAGGACGTTCTG | |
| | ATGAAATCACACAGTGTTCCTGAAGAA | | | CACCGGGACACCCTTGTGAAGGCCTTCCTGGACCAGGTTTTCCAGCTGAAACC | |
| | ATAGATATAGCTGATACAGTACTCAAT | | | TGGCCTCTCCCTGCGGAGCACATTCCTGGCTCAGTTCCTGCTGGTGCTGCATA | |
| | GATGATGATATTGGTGACAGCTGTCAT | | | GAAAGGCCCTGACACTGATCAAGTACATCGAGGATGACACCCAGAAGGGCAAA | |
| | GAAGGCTTTCTTCTCAATGCCATCAGC | | | AAGCCTTTTAAGAGCCTGAGAAACCTGAAGATCGACCTGGATCTGACCGCCGA | |
| | TCACACTTGCAAACCTGTGGCTGTTCC | | | GGGCGACCTGAACATCATCATGGCTCTGGCCGAGAAAATCAAGCCCGGACTGC | |
| | GTTGTAGTAGGTAGCAGTGCAGAGAAA | | | ATAGCTTCATCTTCGGAAGACCTTTCTACACCAGCGTGCAGGAGCGGGACGTG | |
| | GTAAATAAGATAGTCAGAACATTATGC | | | CTGATGACCTTCTGA (SEQ ID NO: 40) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 29 | ATGTCGACTCTTTGCCCACCGCCATCT | 40.73% | AscI | ATGAGCACACTGTGCCCCCCCCCGAGCCCGGCCGTGGCCAAGACAGAGATCGC | 56.48% |
| | CCAGCTGTTGCCAAGACAGAGATTGCT | | [GGCGCGCC]; | CCTGAGCGGCAAGTCCCCTCTGCTGGCCGCCACCTTCGCCTACTGGGACAACA | |
| | TTAAGTGGCAAATCACCTTTATTAGCA | | Not I | TCCTGGGCCCTAGAGTGCGGCACATCTGGGCCCCTAAGACCGAGCAGGTTCTG | |
| | GCTACTTTTGCTTACTGGGACAATATT | | [GCGGCCGC] | CTGAGTGATGGCGAGATAACATTCCTGGCCAACCACACCCTGAACGGCGAGAT | |
| | CTTGGTCCTAGAGTAAGGCACATTTGG | | | CCTGAGAAATGCCGAATCTGGCGCCATCGACGTGAAGTTCTTCGTGCTGTCTG | |
| | GCTCCAAAGACAGAACAGGTACTTCTC | | | AGAAGGGCGTGATCATTGTGTCCCTGATCTTCGACGGCAACTGGAACGGCGAT | |
| | AGTGATGGAGAAATAACTTTTCTTGCC | | | AGAAGCACCTACGGCCTGAGCATCATCCTGCCACAGACCGAACTGTCGTTCTA | |
| | AACCACACTCTAAATGGAGAAATCCTT | | | CCTGCCTCTGCACCGAGTGTGCGTGGACAGACTGACCCACATCATCAGAAAGG | |
| | CGAAATGCAGAGAGTGGTGCTATAGAT | | | GAAGAATCTGGATGCACAAGGAAAGACAGGAGAACGTGCAGAAGATCATCCTG | |
| | GTAAAGTTTTTTGTCTTGTCTGAAAAG | | | GAAGGTACAGAACGGATGGAAGATCAGGGACAGAGCATCATCCCCATGCTGAC | |
| | GGAGTGATTATTGTTTCATTAATCTTT | | | AGGCGAAGTGATCCCTGTGATGGAACTGCTGAGCTCTATGAAAAGCCACAGCG | |
| | GATGGAAACTGGAATGGGGATCGCAGC | | | TGCCTGAGGAAATCGACATCGCTGATACCGTGCTGAACGACGACGATATCGGC | |
| | ACATATGGACTATCAATTATACTTCCA | | | GACAGCTGCCACGAGGGCTTCCTGCTGAACGCCATCAGCAGTCACCTGCAGAC | |
| | CAGACAGAACTTAGTTTCTACCTCCCA | | | ATGCGGCTGTAGCGTCGTGGTGGGCTCCAGCGCCGAGAAAGTGAACAAGATCG | |
| | CTTCATAGAGTGTGTGTTGATAGATTA | | | TGCGCACCCTGTGCCTGTTCCTGACCCCTGCTGAGCGGAAATGCAGCAGACTG | |
| | ACACATATAATCCGGAAAGGAAGAATA | | | TGTGAAGCCGAGAGCTCCTTTAAGTACGAGAGCGGCCTTTTTGTGCAGGGCCT | |
| | TGGATGCATAAGGAAAGACAAGAAAAT | | | GCTGAAGGACAGCACAGGCAGCTTCGTGCTGCCCTTCCGGCAGGTGATGTACG | |
| | GTCCAGAAGATTATCTTAGAAGGCACA | | | CCCCTTATCCTACCACCCACATCGACGTCGACGTGAACACCGTGAAGCAGATG | |
| | GAGAGAATGGAAGATCAGGGTCAGAGT | | | CCTCCTTGCCACGAGCACATCTACAACCAGAGAAGATACATGAGATCCGAGCT | |
| | ATTATTCCAATGCTTACTGGAGAAGTG | | | GACCGCCTTCTGGCGGGCCACAAGCGAGGAAGATATGGCCCAAGACACCATCA | |
| | ATTCCTGTAATGGAACTGCTTTCATCT | | | TCTACACTGATGAGAGTTTCACCCCTGATCTGAACATCTTTCAGGACGTGCTC | |
| | ATGAAATCACACAGTGTTCCTGAAGAA | | | CATCGGGACACCCTGGTGAAAGCTTTCCTGGATCAAGTCTTTCAGCTGAAGCC | |
| | ATAGATATAGCTGATACAGTACTCAAT | | | CGGCCTGTCCCTGCGGTCCACCTTCCTGGCCCAGTTCCTGCTCGTGCTGCACC | |
| | GATGATGATATTGGTGACAGCTGTCAT | | | GGAAGGCCCTGACCCTGATCAAATACATCGAGGACGACACACAGAAAGGCAAA | |
| | GAAGGCTTTCTTCTCAATGCCATCAGC | | | AAGCCTTTCAAGAGCCTGAGAAACCTGAAAATCGATCTGGACCTGACAGCCGA | |
| | TCACACTTGCAAACCTGTGGCTGTTCC | | | GGGCGACCTGAATATCATCATGGCCCTGGCTGAAAAGATTAAGCCCGGACTGC | |
| | GTTGTAGTAGGTAGCAGTGCAGAGAAA | | | ATTCTTTCATCTTCGGCAGACCTTTCTACACCAGCGTGCAGGAGAGAGATGTC | |
| | GTAAATAAGATAGTCAGAACATTATGC | | | CTCATGACCTTTTGA (SEQ ID NO: 41) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 30 | ATGTCGACTCTTTGCCCACCGCCATCT | 40.73% | AscI | ATGAGCACATTGTGTCCTCCACCATCTCCTGCCGTGGCCAAGACCGAAATCGC | 56.41% |
| | CCAGCTGTTGCCAAGACAGAGATTGCT | | [GGCGCGCC]; | CCTGAGCGGCAAGAGCCCCCTGCTCGCCGCCACCTTCGCCTACTGGGACAACA | |
| | TTAAGTGGCAAATCACCTTTATTAGCA | | NotI | TCCTGGGCCCTAGAGTGCGGCACATCTGGGCCCCTAAGACCGAGCAGGTTCTG | |
| | GCTACTTTTGCTTACTGGGACAATATT | | [GCGGCCGC] | CTGAGCGACGGCGAGATAACATTCCTGGCTAATCACACCCTGAATGGCGAGAT | |
| | CTTGGTCCTAGAGTAAGGCACATTTGG | | | CCTGCGGAACGCCGAAAGCGGAGCCATCGACGTGAAGTTCTTCGTGCTGAGCG | |
| | GCTCCAAAGACAGAACAGGTACTTCTC | | | AGAAGGGAGTGATCATCGTGTCCCTGATCTTCGACGGCAACTGGAACGGCGAC | |
| | AGTGATGGAGAAATAACTTTTCTTGCC | | | CGCTCCACCTACGGCCTGTCTATCATCCTGCCTCAGACCGAGCTGAGTTTCTA | |
| | AACCACACTCTAAATGGAGAAATCCTT | | | CCTGCCTCTGCACCGGGTGTGCGTGGACAGACTGACACACATCATCCGGAAAG | |
| | CGAAATGCAGAGAGTGGTGCTATAGAT | | | GCAGAATCTGGATGCACAAGGAACGGCAGGAGAACGTGCAAAAGATCATCCTG | |
| | GTAAAGTTTTTTGTCTTGTCTGAAAAG | | | GAAGGCACCGAGAGAATGGAAGATCAGGGCCAGAGCATCATTCCCATGCTGAC | |
| | GGAGTGATTATTGTTTCATTAATCTTT | | | TGGAGAAGTGATCCCTGTGATGGAACTGCTGAGCAGCATGAAGTCCCACAGCG | |
| | GATGGAAACTGGAATGGGGATCGCAGC | | | TGCCCGAGGAAATCGACATCGCCGACACCGTGCTGAACGACGATGACATAGGA | |
| | ACATATGGACTATCAATTATACTTCCA | | | GATTCATGCCACGAGGGCTTCCTGCTGAACGCCATCAGCTCTCACCTGCAGAC | |
| | CAGACAGAACTTAGTTTCTACCTCCCA | | | ATGCGGCTGTAGCGTCGTGGTGGGCTCTAGCGCCGAAAAGGTGAACAAGATCG | |
| | CTTCATAGAGTGTGTGTTGATAGATTA | | | TCAGAACCCTGTGCCTGTTCCTGACCCCTGCTGAAAGAAAGTGCAGCCGGCTG | |
| | ACACATATAATCCGGAAAGGAAGAATA | | | TGCGAGGCCGAGTCCAGTTTTAAGTACGAGAGCGGCTTGTTTGTGCAGGGACT | |
| | TGGATGCATAAGGAAAGACAAGAAAAT | | | GCTGAAGGACAGCACCGGCAGCTTCGTGCTCCCCTTCAGACAGGTGATGTACG | |
| | GTCCAGAAGATTATCTTAGAAGGCACA | | | CCCCTTATCCTACAACCCACATTGATGTGGATGTTAACACCGTGAAGCAGATG | |
| | GAGAGAATGGAAGATCAGGGTCAGAGT | | | CCTCCATGTCATGAGCACATCTACAACCAGCGTAGATACATGCGGAGCGAGCT | |
| | ATTATTCCAATGCTTACTGGAGAAGTG | | | GACCGCCTTTTGGCGGGCCACAAGCGAGGAAGATATGGCCCAGGATACCATCA | |
| | ATTCCTGTAATGGAACTGCTTTCATCT | | | TCTACACAGACGAGAGCTTCACCCCTGATCTGAATATCTTCCAAGACGTCCTG | |
| | ATGAAATCACACAGTGTTCCTGAAGAA | | | CACAGAGACACCCTCGTGAAGGCCTTCCTGGACCAGGTGTTCCAGCTGAAACC | |
| | ATAGATATAGCTGATACAGTACTCAAT | | | CGGCCTGAGCCTGAGAAGCACCTTCCTCGCTCAGTTCCTGCTGGTGCTGCATA | |
| | GATGATGATATTGGTGACAGCTGTCAT | | | GAAAGGCCCTGACCCTGATCAAGTACATCGAGGACGACACACAGAAAGGAAAA | |
| | GAAGGCTTTCTTCTCAATGCCATCAGC | | | AAGCCCTTCAAGAGCCTGAGAAACCTGAAGATCGACCTGGATCTGACAGCCGA | |
| | TCACACTTGCAAACCTGTGGCTGTTCC | | | GGGCGATCTGAACATCATCATGGCTCTGGCCGAGAAGATCAAGCCTGGCCTCC | |
| | GTTGTAGTAGGTAGCAGTGCAGAGAAA | | | ACTCCTTCATCTTCGGCAGACCTTTTTACACCAGCGTGCAAGAGCGGGACGTG | |
| | GTAAATAAGATAGTCAGAACATTATGC | | | CTCATGACCTTTTGA (SEQ ID NO: 42) | |
| | CTTTTTCTGACTCCAGCAGAGAGAAAA | | | | |
| | TGCTCCAGGTTATGTGAAGCAGAATCA | | | | |
| | TCATTTAAATATGAGTCAGGGCTCTTT | | | | |
| | GTACAAGGCCTGCTAAAGGATTCAACT | | | | |
| | GGAAGCTTTGTGCTGCCTTTCCGGCAA | | | | |
| | GTCATGTATGCTCCATATCCCACCACA | | | | |
| | CACATAGATGTGGATGTCAATACTGTG | | | | |
| | AAGCAGATGCCACCCTGTCATGAACAT | | | | |
| | ATTTATAATCAGCGTAGATACATGAGA | | | | |
| | TCCGAGCTGACAGCCTTCTGGAGAGCC | | | | |
| | ACTTCAGAAGAAGACATGGCTCAGGAT | | | | |
| | ACGATCATCTACACTGACGAAAGCTTT | | | | |
| | ACTCCTGATTTGAATATTTTTCAAGAT | | | | |
| | GTCTTACACAGAGACACTCTAGTGAAA | | | | |
| | GCCTTCCTGGATCAGGTCTTTCAGCTG | | | | |
| | AAACCTGGCTTATCTCTCAGAAGTACT | | | | |
| | TTCCTTGCACAGTTTCTACTTGTCCTT | | | | |
| | CACAGAAAAGCCTTGACACTAATAAAA | | | | |
| | TATATAGAAGACGATACGCAGAAGGGA | | | | |
| | AAAAAGCCCTTTAAATCTCTTCGGAAC | | | | |
| | CTGAAGATAGACCTTGATTTAACAGCA | | | | |
| | GAGGGCGATCTTAACATAATAATGGCT | | | | |
| | CTGGCTGAGAAAATTAAACCAGGCCTA | | | | |
| | CACTCTTTTATCTTTGGAAGACCTTTC | | | | |
| | TACACTAGTGTGCAAGAACGAGATGTT | | | | |
| | CTAATGACTTTTTAA (SEQ ID | | | | |
| | NO: 89) | | | | |
| gene 31 | ATGTCTACACTCTGTCCTCCACCTAGC | 56.29% | AscI | ATGAGCACCCTGTGCCCCCCCCCCAGCCCAGCCGTGGCCAAGACCGAGATAGC | 56.48% |
| | CCTGCTGTGGCCAAGACAGAAATCGCC | | [GGCGCGCC]; | TCTGAGCGGAAAAAGCCCTCTGCTGGCCGCCACCTTCGCCTACTGGGACAACA | |
| | CTGAGCGGAAAAAGCCCCCTGCTGGCC | | NotI | TCCTGGGGCCTAGAGTCAGACACATCTGGGCCCCTAAGACCGAGCAGGTGCTG | |
| | GCCACCTTCGCCTACTGGGACAACATC | | [GCGGCCGC] | CTGAGCGACGGAGAGATCACCTTCCTGGCTAATCACACCCTGAATGGCGAGAT | |
| | CTGGGCCCCAGAGTCAGACACATCTGG | | | CCTGAGAAACGCCGAAAGCGGCGCCATCGACGTGAAGTTCTTCGTGCTGTCTG | |
| | GCCCCTAAGACCGAGCAGGTGCTGCTG | | | AAAAGGGCGTGATCATCGTCAGCCTGATCTTCGACGGCAACTGGAACGGCGAC | |
| | AGCGACGGAGAGATCACCTTCCTGGCC | | | AGAAGCACATACGGCCTGTCTATCATTCTGCCTCAGACAGAGCTGAGTTTTTA | |
| | AACCACACCCTGAATGGCGAGATCCTG | | | CCTGCCTCTGCACCGGGTGTGCGTGGACCGGCTGACCCACATCATTAGAAAGG | |
| | CGGAACGCCGAGTCTGGCGCCATCGAC | | | GAAGAATCTGGATGCACAAGGAACGGCAGGAGAACGTGCAGAAAATCATCCTG | |
| | GTGAAGTTCTTCGTGCTGTCTGAGAAA | | | GAAGGGACCGAGAGAATGGAAGATCAGGGCCAGAGCATCATCCCCATGCTGAC | |
| | GGCGTGATCATTGTGTCCCTCATCTTT | | | CGGCGAAGTGATCCCTGTGATGGAACTGCTGTCTTCTATGAAAAGCCACTCTG | |
| | GACGGCAACTGGAACGGAGATAGAAGC | | | TGCCCGAGGAAATCGATATCGCCGATACAGTGCTGAACGACGACGACATCGGC | |
| | ACCTACGGCCTGTCCATCATCCTGCCC | | | GACTCATGCCACGAGGGCTTCCTTCTGAACGCCATCAGCTCTCACCTGCAGAC | |
| | CAGACAGAGCTGAGCTTCTACCTGCCT | | | CTGTGGCTGCAGCGTGGTCGTGGGCAGCAGCGCCGAGAAAGTGAACAAGATCG | |
| | CTGCACAGAGTGTGCGTGGACAGACTG | | | TGCGGACCCTGTGTCTGTTCCTCACACCTGCCGAGCGGAAGTGCAGTAGACTG | |
| | ACCCACATCATCAGAAAGGGCAGAATC | | | TGCGAGGCCGAATCCAGCTTTAAGTACGAGAGCGGCCTGTTCGTGCAGGGCCT | |
| | TGGATGCACAAGGAACGGCAGGAGAAC | | | GCTGAAAGACAGCACAGGCTCTTTCGTGCTCCCTTTTAGACAGGTGATGTACG | |
| | GTGCAAAAAATCATCCTGGAAGGCACC | | | CCCCTTACCCCACCACACACATTGATGTCGACGTGAACACCGTGAAACAGATG | |
| | GAGAGAATGGAAGATCAGGGCCAGAGC | | | CCTCCATGTCACGAGCACATCTATAACCAGAGAAGATACATGCGGTCCGAGCT | |
| | ATCATCCCCATGCTGACCGGCGAGGTG | | | GACCGCTTTCTGGCGGGCCACAAGCGAAGAGGACATGGCTCAGGACACAATCA | |
| | ATCCCTGTGATGGAACTGCTGAGCAGC | | | TCTACACTGATGAGTCCTTCACCCCTGATCTGAACATCTTCCAAGATGTGCTG | |
| | ATGAAGTCCCATTCTGTCCCCGAGGAA | | | CACAGGGACACCCTGGTGAAGGCCTTCCTGGATCAGGTCTTTCAGCTGAAGCC | |
| | ATCGACATCGCCGACACCGTGCTGAAC | | | TGGCCTGTCCCTGCGCTCCACCTTCCTGGCCCAATTTCTGCTCGTGCTGCACA | |
| | GACGATGATATCGGCGATAGCTGCCAC | | | GAAAGGCCCTGACCCTGATTAAGTACATCGAGGACGATACCCAGAAGGGCAAG | |
| | GAGGGCTTCCTGCTGAACGCCATCAGC | | | AAGCCTTTCAAGTCCCTGCGGAATCTGAAGATCGACCTGGACCTGACCGCCGA | |
| | TCTCACCTGCAGACCTGCGGCTGCAGC | | | GGGCGATCTGAACATCATCATGGCCCTGGCCGAGAAGATCAAGCCCGGCCTCC | |
| | GTGGTGGTCGGCTCTTCCGCCGAAAAG | | | ACAGCTTCATCTTCGGCAGACCTTTCTACACCAGCGTGCAGGAGAGAGATGTG | |
| | GTGAACAAGATCGTGCGGACCCTGTGC | | | CTGATGACATTTTGA (SEQ ID NO: 43) | |
| | CTGTTCCTGACTCCTGCCGAAAGAAAG | | | | |
| | TGCTCTAGACTGTGTGAAGCCGAGAGC | | | | |
| | AGCTTCAAATACGAGTCCGGTCTTTTT | | | | |
| | GTGCAGGGGCTGCTGAAGGACAGCACA | | | | |
| | GGCAGCTTCGTGCTTCCATTCAGACAG | | | | |
| | GTGATGTACGCCCCTTACCCCACAACA | | | | |
| | CACATTGATGTGGACGTGAACACCGTG | | | | |
| | AAGCAGATGCCTCCTTGCCACGAGCAC | | | | |
| | ATCTACAACCAGCGGAGATACATGCGG | | | | |
| | AGCGAGCTGACAGCCTTCTGGCGGGCC | | | | |
| | ACAAGCGAGGAAGATATGGCCCAGGAC | | | | |
| | ACCATCATCTACACCGACGAGAGCTTC | | | | |
| | ACCCCTGATCTGAATATCTTCCAAGAC | | | | |
| | GTCCTGCACCGCGACACACTCGTGAAA | | | | |
| | GCCTTTCTCGACCAGGTTTTCCAGCTG | | | | |
| | AAACCTGGCCTGAGTCTGAGATCCACC | | | | |
| | TTCCTGGCTCAATTTCTGCTGGTGCTC | | | | |
| | CACCGGAAGGCCCTGACCCTGATCAAG | | | | |
| | TACATCGAGGACGACACCCAGAAGGGC | | | | |
| | AAGAAGCCTTTCAAGTCTCTGAGAAAC | | | | |
| | CTGAAGATCGACCTGGACCTGACAGCT | | | | |
| | GAGGGCGACCTGAATATCATCATGGCC | | | | |
| | CTTGCTGAGAAGATCAAGCCCGGCCTG | | | | |
| | CACAGCTTCATCTTCGGCAGACCTTTT | | | | |
| | TATACCAGCGTGCAGGAGAGAGATGTG | | | | |
| | CTGATGACCTTCTGA (SEQ ID | | | | |
| | NO: 90) | | | | |
| gene 32 | ATGAGCACACTGTGCCCTCCACCTAGC | 56.15% | AscI | ATGTCTACACTGTGTCCTCCACCTAGCCCCGCCGTGGCCAAGACAGAAATCGC | 56.20% |
| | CCTGCCGTGGCCAAGACCGAGATCGCC | | [GGCGCGCC]; | CCTGAGCGGAAAGTCCCCTCTGCTGGCCGCCACATTTGCCTACTGGGACAACA | |
| | CTTTCCGGCAAGAGCCCCCTGCTGGCC | | NotI | TACTGGGCCCTAGAGTGCGGCACATCTGGGCCCCTAAGACCGAGCAGGTGCTG | |
| | GCCACATTCGCCTACTGGGACAACATC | | [GCGGCCGC] | CTGAGCGACGGCGAGATCACCTTCCTGGCCAACCACACCCTGAACGGCGAAAT | |
| | CTGGGACCTAGAGTGCGGCACATTTGG | | | CCTGAGAAACGCCGAAAGCGGCGCCATCGACGTGAAGTTCTTCGTGCTGAGCG | |
| | GCCCCTAAGACCGAGCAGGTCCTGCTG | | | AGAAAGGCGTGATCATCGTGTCCCTGATCTTCGACGGCAACTGGAACGGCGAT | |
| | AGTGATGGCGAAATCACCTTCCTGGCC | | | AGAAGCACCTACGGCCTGAGCATCATTCTGCCTCAGACCGAGCTGAGCTTCTA | |
| | AACCACACCCTGAACGGCGAGATCCTG | | | CCTGCCTCTTCATAGAGTGTGCGTGGACAGACTGACCCACATTATTAGAAAGG | |
| | CGGAACGCCGAGAGCGGTGCTATCGAT | | | GAAGAATCTGGATGCACAAGGAACGGCAGGAGAACGTGCAGAAAATCATCCTG | |
| | GTGAAGTTCTTCGTGCTGAGCGAGAAG | | | GAAGGGACCGAGCGGATGGAAGATCAGGGCCAGAGCATCATCCCCATGCTGAC | |
| | GGCGTGATCATCGTGTCCCTCATCTTC | | | AGGCGAGGTGATCCCTGTGATGGAACTGCTGTCCAGCATGAAGTCTCACAGCG | |
| | GACGGCAACTGGAACGGCGACAGATCT | | | TGCCCGAGGAAATCGATATCGCCGATACAGTGCTGAACGACGATGACATCGGC | |
| | ACATACGGCCTGTCTATCATCCTGCCT | | | GACAGCTGCCACGAGGGCTTCCTGCTGAATGCCATTTCTAGCCACCTGCAGAC | |
| | CAGACCGAACTGTCCTTCTACCTGCCT | | | ATGCGGATGTAGCGTCGTGGTGGGCTCTAGCGCCGAGAAGGTGAACAAGATCG | |
| | CTGCACCGGGTGTGCGTGGACCGGCTG | | | TGCGGACCCTGTGCCTGTTCCTGACACCTGCTGAACGCAAGTGCAGCAGACTG | |
| | ACTCACATCATCAGAAAGGGCAGAATC | | | TGTGAAGCCGAAAGCTCTTTTAAGTACGAGAGCGGCCTCTTCGTCCAGGGCCT | |
| | TGGATGCACAAGGAACGGCAGGAGAAC | | | GCTGAAGGACAGCACCGGCTCTTTTGTGCTGCCCTTCAGACAGGTGATGTACG | |
| | GTGCAAAAGATCATTCTGGAAGGTACA | | | CCCCTTACCCCACCACCCACATCGACGTCGACGTGAATACCGTGAAACAGATG | |
| | GAGAGAATGGAAGATCAGGGCCAGAGC | | | CCTCCTTGCCACGAGCACATCTACAACCAGAGAAGATACATGAGAAGCGAGCT | |
| | ATTATCCCTATGCTGACAGGCGAGGTG | | | GACAGCCTTCTGGCGGGCCACCTCTGAAGAGGATATGGCCCAGGACACAATCA | |
| | ATCCCCGTGATGGAACTGTTGTCCTCC | | | TCTACACCGACGAGAGCTTCACCCCTGATCTGAACATCTTCCAAGACGTGCTG | |
| | ATGAAGTCCCACTCTGTTCCTGAGGAA | | | CACAGAGATACCCTGGTGAAGGCTTTTCTGGACCAGGTTTTCCAGCTGAAGCC | |
| | ATCGACATCGCCGACACAGTGCTGAAC | | | TGGACTGTCTCTGAGATCTACCTTCCTTGCTCAATTTCTGCTGGTCCTCCACC | |
| | GACGACGATATCGGCGACAGCTGCCAC | | | GGAAAGCCCTGACACTGATCAAGTACATCGAGGACGACACCCAGAAGGGCAAG | |
| | GAGGGCTTCCTGCTGAACGCCATCAGC | | | AAGCCCTTCAAGAGCCTGAGGAACCTGAAAATCGACCTGGATCTGACCGCCGA | |
| | AGCCACCTGCAGACCTGTGGCTGTAGC | | | GGGCGACCTGAACATCATCATGGCCCTGGCTGAAAAGATCAAGCCTGGCCTGC | |
| | GTGGTCGTGGGCTCTAGCGCCGAAAAG | | | ACAGTTTCATCTTCGGCAGACCTTTCTACACCAGCGTGCAGGAGCGGGACGTG | |
| | GTGAACAAGATCGTGCGGACCCTGTGT | | | CTGATGACCTTCTGA (SEQ ID NO: 44) | |
| | CTGTTCCTGACACCTGCTGAGAGAAAG | | | | |
| | TGCAGCAGACTGTGCGAGGCCGAGTCT | | | | |
| | AGCTTTAAGTACGAGAGCGGCCTGTTC | | | | |
| | GTGCAGGGCCTCCTGAAGGACAGCACC | | | | |
| | GGCAGCTTTGTGCTGCCCTTCAGACAG | | | | |
| | GTGATGTACGCCCCTTACCCCACCACC | | | | |
| | CACATCGACGTGGACGTGAACACCGTG | | | | |
| | AAGCAGATGCCTCCGTGCCATGAGCAC | | | | |
| | ATCTACAACCAGAGAAGATACATGAGA | | | | |
| | AGCGAGCTGACCGCTTTCTGGCGGGCC | | | | |
| | ACCTCTGAGGAAGATATGGCCCAGGAC | | | | |
| | ACCATCATCTATACAGACGAGAGCTTC | | | | |
| | ACCCCTGATCTGAATATTTTCCAAGAT | | | | |
| | GTGCTGCACAGAGATACACTTGTGAAA | | | | |
| | GCCTTCCTCGACCAGGTGTTCCAGCTG | | | | |
| | AAGCCTGGCCTGTCTCTGCGGAGCACC | | | | |
| | TTTCTGGCACAGTTCCTGCTGGTGCTG | | | | |
| | CATAGAAAGGCCCTGACCTTGATCAAG | | | | |
| | TACATCGAGGATGACACCCAGAAAGGA | | | | |
| | AAGAAACCTTTCAAGAGCCTGAGAAAC | | | | |
| | CTGAAAATCGACCTGGACCTGACGGCC | | | | |
| | GAAGGCGATCTGAATATCATCATGGCC | | | | |
| | CTGGCCGAGAAGATCAAGCCCGGCCTG | | | | |
| | CACAGCTTCATCTTTGGAAGACCTTTT | | | | |
| | TACACCAGCGTGCAGGAGCGGGACGTG | | | | |
| | CTGATGACATTTTGA (SEQ ID | | | | |
| | NO: 91) | | | | |
| gene 33 | ATGAGCACCCTGTGTCCTCCACCTAGC | 55.88% | AscI | ATGAGCACCCTGTGCCCCCCCCCCAGCCCCGCCGTGGCCAAGACCGAGATCGC | 56.34% |
| | CCCGCCGTGGCCAAGACCGAGATCGCC | | [GGCGCGCC]; | CCTGTCTGGCAAGTCCCCTCTGCTTGCCGCTACCTTCGCCTACTGGGACAACA | |
| | CTGTCTGGAAAGTCCCCTCTGCTGGCC | | NotI | TCCTGGGCCCTAGAGTGCGGCACATCTGGGCCCCTAAGACCGAGCAGGTCCTG | |
| | GCTACATTCGCCTACTGGGACAACATC | | [GCGGCCGC] | CTGAGCGACGGCGAAATCACCTTCCTGGCCAACCACACCCTGAACGGCGAGAT | |
| | CTGGGACCTAGAGTGCGGCACATCTGG | | | CCTGCGGAACGCCGAGAGCGGCGCCATCGACGTGAAGTTCTTCGTGCTGAGCG | |
| | GCCCCTAAGACCGAGCAGGTGCTCCTG | | | AGAAGGGCGTGATCATCGTGTCCCTGATCTTCGACGGAAATTGGAACGGCGAC | |
| | AGTGATGGCGAGATAACATTTCTGGCC | | | AGATCCACATACGGCCTGAGCATCATCCTGCCTCAGACAGAGCTGTCCTTTTA | |
| | AACCACACCCTCAACGGCGAGATCCTG | | | CCTGCCCCTGCACCGGGTGTGCGTGGATAGACTGACACACATCATTAGAAAGG | |
| | AGAAACGCCGAAAGCGGCGCCATCGAC | | | GAAGAATCTGGATGCACAAGGAACGGCAGGAGAACGTGCAGAAAATCATCCTG | |
| | GTGAAGTTCTTCGTGCTGTCTGAAAAG | | | GAAGGTACAGAGAGAATGGAAGATCAGGGACAGTCTATCATCCCCATGCTGAC | |
| | GGCGTGATCATCGTGTCCCTGATCTTC | | | CGGCGAGGTGATCCCCGTGATGGAACTGCTGAGTTCTATGAAGTCCCACAGCG | |
| | GACGGCAACTGGAACGGCGACAGAAGC | | | TGCCTGAGGAAATCGACATCGCCGACACCGTGCTGAACGACGATGACATAGGA | |
| | ACGTACGGCCTGTCCATCATCCTGCCC | | | GATAGCTGCCACGAGGGCTTCCTGCTGAATGCCATAAGCAGCCACCTGCAGAC | |
| | CAGACCGAGCTGTCTTTCTACCTGCCT | | | CTGTGGCTGCAGCGTCGTGGTGGGCAGCAGCGCCGAAAAGGTGAACAAGATCG | |
| | CTGCACCGGGTGTGCGTGGATAGACTG | | | TTAGAACACTGTGCCTGTTTCTGACCCCTGCTGAGCGGAAGTGCAGCAGACTG | |
| | ACCCACATTATTAGAAAGGGCAGAATC | | | TGTGAAGCCGAGTCTAGCTTCAAGTACGAGTCCGGCCTGTTCGTGCAAGGCCT | |
| | TGGATGCACAAGGAACGCCAGGAGAAC | | | GCTCAAGGACAGCACAGGCTCCTTCGTGCTGCCTTTTAGACAGGTGATGTACG | |
| | GTGCAGAAGATCATCCTGGAAGGTACA | | | CCCCTTACCCCACCACCCATATCGACGTGGACGTGAACACCGTCAAGCAGATG | |
| | GAGCGGATGGAAGATCAGGGCCAGAGC | | | CCTCCATGTCACGAGCACATCTACAACCAGCGTAGATACATGAGAAGCGAGCT | |
| | ATCATCCCCATGCTGACCGGCGAAGTG | | | TACAGCTTTCTGGCGGGCCACCTCTGAAGAGGACATGGCCCAGGACACCATCA | |
| | ATCCCTGTGATGGAACTGCTGAGTTCT | | | TCTACACCGACGAGAGCTTCACCCCTGACCTGAACATTTTTCAAGATGTGCTG | |
| | ATGAAAAGCCACAGCGTGCCGGAAGAG | | | CACAGAGATACCCTGGTGAAAGCCTTCCTGGATCAGGTGTTCCAGCTGAAACC | |
| | ATCGATATCGCCGACACCGTCCTTAAC | | | TGGACTGAGCCTGAGAAGCACCTTCTTGGCACAGTTCCTCCTGGTCCTGCACA | |
| | GACGACGACATAGGAGATAGCTGCCAC | | | GAAAGGCCCTGACCCTCATCAAGTACATCGAGGATGATACCCAGAAGGGCAAA | |
| | GAGGGCTTCCTTCTGAACGCCATCAGC | | | AAGCCCTTCAAGAGCCTGAGAAACCTGAAGATCGATCTGGACCTGACAGCCGA | |
| | TCTCACCTGCAGACATGCGGCTGCAGC | | | GGGCGACCTGAACATCATCATGGCTCTGGCTGAAAAAATCAAGCCTGGCCTGC | |
| | GTCGTGGTCGGCTCTAGCGCCGAAAAA | | | ATAGCTTCATCTTCGGCAGACCTTTCTATACAAGCGTGCAGGAGCGGGACGTG | |
| | GTGAACAAGATCGTGCGGACCCTGTGC | | | CTGATGACATTCTGA (SEQ ID NO: 45) | |
| | CTGTTCCTGACACCTGCCGAGAGAAAG | | | | |
| | TGCTCTAGACTGTGCGAGGCCGAGTCC | | | | |
| | AGCTTCAAGTACGAGAGCGGCCTGTTT | | | | |
| | GTTCAAGGACTGCTGAAGGACAGCACC | | | | |
| | GGCAGCTTTGTGCTCCCTTTTAGACAG | | | | |
| | GTGATGTACGCCCCTTACCCCACCACC | | | | |
| | CACATCGACGTTGACGTGAATACCGTG | | | | |
| | AAACAGATGCCTCCTTGTCACGAGCAC | | | | |
| | ATCTACAACCAGAGAAGATACATGAGA | | | | |
| | TCTGAGCTGACCGCCTTCTGGCGGGCC | | | | |
| | ACCAGCGAGGAAGATATGGCCCAGGAC | | | | |
| | ACCATCATCTACACCGACGAGAGCTTC | | | | |
| | ACCCCTGATCTGAACATCTTTCAGGAT | | | | |
| | GTCCTGCACCGCGACACCCTGGTCAAA | | | | |
| | GCCTTTCTGGACCAGGTGTTCCAGCTG | | | | |
| | AAACCCGGACTGTCTCTGCGGAGCACC | | | | |
| | TTCTTGGCTCAATTTCTCCTGGTGCTG | | | | |
| | CACAGAAAGGCCCTGACACTGATCAAG | | | | |
| | TACATCGAGGATGATACACAGAAAGGC | | | | |
| | AAAAAGCCCTTCAAGAGCCTGAGAAAT | | | | |
| | CTGAAGATCGACCTGGACCTGACAGCC | | | | |
| | GAGGGCGATCTGAACATCATCATGGCC | | | | |
| | CTGGCTGAGAAGATTAAGCCTGGCCTC | | | | |
| | CATTCTTTCATCTTCGGCAGACCTTTC | | | | |
| | TACACCAGCGTGCAGGAGCGGGACGTG | | | | |
| | CTGATGACATTCTGA (SEQ ID | | | | |
| | NO: 92) | | | | |
| gene 34 | ATGAGCACCCTGTGTCCTCCTCCATCT | 56.09% | AscI | ATGAGCACACTGTGTCCTCCTCCGAGCCCTGCTGTGGCCAAGACCGAGATCGC | 56.62% |
| | CCAGCCGTGGCCAAGACCGAGATCGCC | | [GGCGCGCC]; | CCTGAGCGGCAAGTCCCCACTCCTGGCTGCTACATTCGCCTACTGGGACAACA | |
| | CTGTCCGGCAAGAGCCCTCTGCTGGCC | | NotI | TCCTGGGCCCTAGAGTGCGGCACATCTGGGCCCCCAAGACAGAACAGGTTCTG | |
| | GCTACATTCGCCTACTGGGACAACATC | | [GCGGCCGC] | CTGAGTGATGGCGAGATCACCTTCCTCGCCAATCACACCCTGAACGGCGAAAT | |
| | CTGGGACCTAGAGTGCGGCACATCTGG | | | CCTGAGAAACGCCGAGAGCGGCGCCATCGATGTGAAATTCTTCGTGCTGAGCG | |
| | GCCCCTAAGACAGAGCAGGTGCTGCTG | | | AGAAGGGCGTGATCATCGTGTCCCTGATCTTCGACGGCAACTGGAACGGCGAT | |
| | AGTGATGGCGAGATCACCTTCCTGGCC | | | AGAAGCACCTACGGCCTGAGCATCATCCTGCCCCAGACCGAGCTGAGCTTCTA | |
| | AACCACACCCTGAATGGAGAAATCCTG | | | CCTGCCTCTGCACCGGGTGTGCGTGGACAGACTGACACACATCATTAGAAAGG | |
| | AGAAACGCCGAGAGTGGCGCCATCGAT | | | GCAGAATCTGGATGCACAAGGAACGGCAGGAGAACGTGCAAAAGATCATTCTG | |
| | GTGAAGTTCTTCGTGCTGTCTGAAAAG | | | GAAGGGACCGAGCGGATGGAAGATCAGGGCCAGAGCATCATCCCTATGCTGAC | |
| | GGCGTGATCATCGTCAGCCTGATCTTC | | | AGGAGAAGTGATCCCCGTGATGGAACTGCTGTCTAGCATGAAATCTCACAGCG | |
| | GACGGCAACTGGAACGGCGACAGAAGC | | | TGCCCGAGGAAATCGACATCGCCGACACCGTGCTGAACGACGACGACATCGGC | |
| | ACATACGGCCTGAGCATCATCCTGCCC | | | GACAGCTGCCATGAGGGCTTCCTTCTCAACGCCATCAGCAGCCACCTGCAGAC | |
| | CAGACAGAGCTGTCTTTTTACCTGCCT | | | CTGTGGCTGCAGCGTGGTGGTCGGATCTTCTGCCGAAAAGGTGAACAAGATCG | |
| | CTGCACAGAGTGTGCGTGGACCGGCTG | | | TGCGGACCCTGTGCCTGTTCCTGACCCCTGCCGAACGGAAGTGCAGCAGACTG | |
| | ACCCACATCATTAGAAAGGGCAGAATC | | | TGCGAGGCCGAGAGCAGCTTTAAGTACGAGTCTGGCCTGTTCGTGCAGGGCCT | |
| | TGGATGCACAAGGAAAGACAGGAGAAC | | | GCTGAAGGACAGCACAGGCAGCTTTGTGCTGCCTTTTAGACAGGTGATGTACG | |
| | GTGCAGAAGATCATCCTGGAAGGTACA | | | CCCCTTACCCCACCACCCACATCGACGTCGACGTGAACACCGTGAAGCAGATG | |
| | GAGAGAATGGAAGATCAGGGACAGAGC | | | CCTCCATGTCACGAGCACATCTACAACCAGCGGAGATACATGAGATCCGAGCT | |
| | ATCATCCCCATGCTGACCGGCGAAGTG | | | GACAGCCTTCTGGCGGGCCACCAGCGAAGAGGATATGGCCCAGGATACAATCA | |
| | ATCCCTGTGATGGAACTGCTGAGCAGC | | | TCTATACAGACGAGTCCTTCACCCCTGATCTGAACATCTTTCAGGACGTTCTG | |
| | ATGAAAAGCCATTCTGTGCCCGAGGAA | | | CACAGAGATACCCTGGTGAAGGCTTTCCTGGACCAAGTGTTCCAGCTGAAACC | |
| | ATCGACATCGCCGACACAGTGCTGAAC | | | TGGACTGAGCCTGCGGAGCACCTTTCTGGCCCAGTTCCTGCTGGTCCTGCACA | |
| | GACGACGATATCGGCGATAGCTGCCAC | | | GAAAGGCCCTGACCCTGATCAAGTACATCGAGGACGATACCCAGAAAGGCAAA | |
| | GAGGGATTCCTGCTTAATGCCATCAGC | | | AAGCCTTTCAAGAGCCTGAGAAATCTGAAGATCGACCTGGATCTGACCGCCGA | |
| | AGCCACCTGCAGACCTGTGGCTGTAGC | | | GGGAGATCTGAATATCATCATGGCCCTGGCCGAGAAAATCAAGCCCGGCCTCC | |
| | GTGGTCGTGGGCAGCTCCGCCGAGAAG | | | ATTCTTTCATCTTCGGCAGACCCTTCTACACATCTGTGCAGGAGCGCGACGTG | |
| | GTGAACAAGATCGTGAGGACCCTCTGC | | | CTGATGACCTTCTGA (SEQ ID NO: 46) | |
| | CTGTTCCTGACACCTGCTGAAAGAAAG | | | | |
| | TGCAGCAGACTGTGCGAGGCCGAGTCC | | | | |
| | AGCTTCAAGTACGAGAGCGGCCTCTTC | | | | |
| | GTGCAGGGCCTGCTGAAGGACAGCACC | | | | |
| | GGCTCCTTCGTGCTGCCTTTTAGACAG | | | | |
| | GTGATGTACGCCCCTTACCCCACCACC | | | | |
| | CACATTGACGTGGACGTGAACACCGTG | | | | |
| | AAGCAGATGCCTCCGTGCCACGAGCAC | | | | |
| | ATCTACAACCAGCGCAGATACATGCGG | | | | |
| | AGCGAGCTGACCGCCTTCTGGCGGGCC | | | | |
| | ACATCTGAGGAAGATATGGCTCAAGAT | | | | |
| | ACCATCATCTACACCGACGAGAGCTTC | | | | |
| | ACCCCTGATCTGAACATCTTCCAGGAC | | | | |
| | GTGCTGCATAGAGATACCCTGGTGAAA | | | | |
| | GCTTTCCTTGATCAGGTTTTCCAACTG | | | | |
| | AAGCCTGGCCTGAGCCTGAGAAGCACC | | | | |
| | TTCCTGGCTCAGTTCCTGCTGGTGCTT | | | | |
| | CACCGGAAGGCCCTAACCCTGATCAAG | | | | |
| | TACATCGAGGATGACACCCAGAAAGGC | | | | |
| | AAAAAGCCTTTTAAGTCCCTGCGGAAC | | | | |
| | CTGAAAATCGACCTGGACCTCACAGCC | | | | |
| | GAGGGAGATCTGAACATCATCATGGCC | | | | |
| | CTGGCCGAAAAGATAAAGCCCGGCCTG | | | | |
| | CACAGCTTCATCTTTGGCAGACCTTTC | | | | |
| | TACACAAGCGTGCAGGAGCGGGACGTG | | | | |
| | CTGATGACCTTCTGA (SEQ ID | | | | |
| | NO: 93) | | | | |
| gene 35 | ATGAGCACCCTCTGTCCTCCACCTAGC | 56.02% | AscI | ATGAGCACCCTGTGTCCTCCACCCAGCCCTGCCGTGGCCAAGACAGAGATCGC | 56.62% |
| | CCTGCTGTGGCCAAGACCGAAATTGCC | | [GGCGCGCC]; | CCTGTCTGGAAAGAGCCCCCTGCTGGCCGCTACCTTCGCCTACTGGGACAACA | |
| | CTGAGCGGAAAGTCTCCTCTGTTGGCT | | NotI | TCCTGGGCCCTAGAGTGCGGCACATCTGGGCCCCTAAGACAGAGCAGGTCCTG | |
| | GCTACATTCGCCTACTGGGACAACATC | | [GCGGCCGC] | CTGAGCGACGGCGAAATCACCTTCCTGGCTAATCACACCCTTAATGGAGAAAT | |
| | CTGGGCCCTAGAGTGCGGCACATCTGG | | | CCTGAGAAACGCCGAATCCGGCGCCATCGACGTGAAGTTCTTCGTGCTGAGCG | |
| | GCCCCTAAGACAGAGCAGGTGCTGCTG | | | AGAAAGGCGTGATCATCGTGTCCCTGATCTTTGATGGAAATTGGAACGGCGAC | |
| | AGTGATGGCGAAATCACCTTCCTGGCC | | | AGAAGCACATACGGCCTGAGCATCATCCTGCCTCAGACCGAGCTGTCTTTTTA | |
| | AACCACACCCTGAACGGCGAGATCCTG | | | CCTGCCTCTGCACAGAGTGTGCGTGGACCGGCTGACCCACATCATCAGAAAGG | |
| | AGAAACGCCGAAAGCGGCGCCATCGAC | | | GCAGAATCTGGATGCACAAGGAACGGCAGGAGAACGTGCAGAAAATCATTCTG | |
| | GTGAAGTTCTTCGTGCTGTCTGAAAAG | | | GAAGGCACCGAGCGGATGGAAGATCAGGGCCAGAGCATCATCCCCATGCTGAC | |
| | GGTGTTATCATTGTGTCCCTGATCTTT | | | CGGCGAGGTGATCCCCGTGATGGAACTGCTGTCTAGCATGAAATCTCACTCTG | |
| | GACGGCAACTGGAACGGCGACAGATCT | | | TGCCTGAGGAAATCGACATCGCCGACACAGTGCTGAACGACGACGACATCGGC | |
| | ACATACGGCCTGTCCATCATCCTGCCT | | | GATAGCTGCCACGAGGGCTTCCTGCTGAACGCCATCAGCAGCCACCTGCAGAC | |
| | CAGACCGAGCTGTCTTTCTACCTGCCT | | | ATGCGGCTGCAGCGTGGTCGTGGGAAGCAGCGCCGAAAAGGTGAACAAGATCG | |
| | CTGCACAGAGTGTGCGTGGACCGGCTG | | | TGCGGACCCTCTGTCTGTTCCTGACGCCCGCCGAGAGAAAGTGCAGCAGACTG | |
| | ACTCATATCATCAGAAAGGGAAGAATC | | | TGTGAAGCCGAGAGCAGCTTTAAGTACGAGTCTGGCCTGTTTGTGCAGGGCCT | |
| | TGGATGCACAAGGAAAGACAGGAGAAC | | | GCTGAAGGACAGCACCGGCTCTTTCGTGCTGCCCTTCAGACAGGTGATGTACG | |
| | GTGCAGAAGATCATCCTGGAAGGTACA | | | CCCCTTACCCCACCACACACATTGACGTGGACGTCAACACCGTGAAACAGATG | |
| | GAGAGAATGGAAGATCAGGGCCAGAGC | | | CCTCCTTGCCATGAACACATCTACAACCAGCGGAGATACATGCGGAGCGAGCT | |
| | ATCATCCCCATGCTGACAGGCGAGGTG | | | GACCGCCTTCTGGCGGGCCACCTCTGAGGAAGATATGGCCCAGGACACCATCA | |
| | ATCCCTGTGATGGAACTGCTGAGCAGC | | | TCTATACAGACGAGTCCTTCACCCCTGATCTGAATATCTTCCAAGATGTTCTC | |
| | ATGAAGTCCCACAGCGTCCCCGAGGAA | | | CACAGGGACACCCTGGTGAAGGCTTTTCTCGACCAGGTGTTCCAGCTGAAACC | |
| | ATCGACATCGCCGACACAGTGCTGAAC | | | TGGCCTGAGCCTGCGGAGCACCTTTCTGGCCCAATTTCTGCTCGTGCTGCACA | |
| | GACGACGATATCGGCGATTCATGCCAC | | | GAAAGGCCCTGACCCTGATCAAATACATCGAGGACGATACACAGAAGGGCAAG | |
| | GAGGGCTTCCTGCTGAATGCAATCAGC | | | AAGCCTTTCAAGTCCCTGAGAAACCTGAAGATCGACCTGGATCTGACAGCCGA | |
| | AGCCACCTGCAGACCTGCGGCTGTTCT | | | GGGCGACCTGAACATCATTATGGCTCTGGCCGAGAAGATCAAGCCTGGACTCC | |
| | GTGGTGGTGGGCAGCAGCGCCGAAAAA | | | ACAGCTTCATCTTCGGCCGCCCCTTCTACACCAGCGTGCAAGAGAGAGACGTG | |
| | GTGAACAAGATCGTGCGCACCCTGTGC | | | CTGATGACCTTCTGA (SEQ ID NO: 47) | |
| | CTGTTTTTGACCCCTGCCGAGCGGAAG | | | | |
| | TGCAGCAGACTGTGTGAAGCCGAGAGC | | | | |
| | TCTTTCAAGTACGAGAGCGGCCTGTTC | | | | |
| | GTTCAAGGCCTGCTGAAGGACAGCACC | | | | |
| | GGCAGCTTTGTGCTGCCCTTCCGGCAG | | | | |
| | GTGATGTACGCCCCTTACCCCACCACC | | | | |
| | CACATCGACGTCGACGTGAACACCGTG | | | | |
| | AAGCAGATGCCTCCGTGCCACGAGCAC | | | | |
| | ATCTACAACCAGCGGAGATACATGCGG | | | | |
| | TCCGAGCTGACAGCCTTCTGGCGGGCC | | | | |
| | ACCAGCGAAGAGGACATGGCCCAGGAC | | | | |
| | ACCATCATCTACACTGATGAGTCCTTC | | | | |
| | ACACCTGATCTGAATATCTTCCAAGAC | | | | |
| | GTGCTTCACAGAGACACCCTGGTGAAA | | | | |
| | GCTTTTCTCGACCAGGTTTTCCAGCTG | | | | |
| | AAGCCCGGCCTGAGCCTGAGATCTACC | | | | |
| | TTCCTGGCTCAATTTCTGCTCGTGCTG | | | | |
| | CACAGAAAGGCCCTGACGCTGATCAAG | | | | |
| | TATATCGAGGACGACACGCAGAAAGGC | | | | |
| | AAGAAACCCTTCAAAAGCCTGCGGAAC | | | | |
| | CTGAAAATTGACCTGGACCTGACCGCC | | | | |
| | GAGGGCGACCTGAACATCATCATGGCC | | | | |
| | CTGGCCGAGAAGATCAAGCCTGGACTG | | | | |
| | CATAGCTTCATCTTCGGCAGACCTTTT | | | | |
| | TACACCTCTGTGCAGGAGCGGGACGTG | | | | |
| | CTCATGACCTTTTGA (SEQ ID | | | | |
| | NO: 94) | | | | |
| gene 36 | ATGAGCACCCTGTGTCCTCCTCCAAGC | 56.43% | AscI | ATGAGCACACTGTGCCCCCCCCCTTCTCCTGCCGTGGCCAAGACCGAGATTGC | 55.99% |
| | CCTGCCGTGGCCAAGACAGAGATCGCC | | [GGCGCGCC]; | CCTGTCCGGCAAGTCCCCTCTGTTGGCCGCCACATTTGCCTACTGGGACAACA | |
| | CT7AGCGGAAAGTCCCCTCTGCTGGCC | | Not I | TCCTGGGCCCTAGAGTGCGGCACATTTGGGCCCCTAAGACAGAACAGGTGCTG | |
| | GCCACATTTGCCTACTGGGACAACATC | | [GCGGCCGC] | CTGAGTGATGGCGAGATCACCTTTCTGGCCAACCACACCCTGAATGGCGAAAT | |
| | CTGGGACCTAGAGTGCGGCACATTTGG | | | CCTGAGAAACGCCGAGAGCGGAGCCATCGACGTGAAGTTCTTCGTGCTGTCTG | |
| | GCCCCAAAGACCGAGCAGGTGCTGCTG | | | AGAAGGGTGTTATCATCGTGTCCCTGATCTTCGACGGCAACTGGAACGGCGAC | |
| | AGCGACGGCGAAATCACCTTCCTGGCT | | | AGATCTACCTACGGCCTTTCTATCATCCTGCCCCAGACCGAGCTGAGCTTCTA | |
| | AATCACACACTGAACGGCGAGATCCTG | | | CCTGCCTCTGCATCGGGTGTGCGTGGACCGGCTGACACACATCATTACAAAGG | |
| | AGGAACGCCGAAAGCGGCGCCATCGAC | | | GGAGAATCTGGATGCACAAGGAACGCCAGGAGAACGTGCAGAAAATCATTCTG | |
| | GTGAAGTTCTTCGTCCTGAGCGAGAAG | | | GAAGGGACCGAAAGAATGGAAGATCAGGGCCAGAGCATCATCCCTATGCTGAC | |
| | GGCGTGATCATTGTGTCCCTGATCTTC | | | AGGAGAGGTGATCCCCGTGATGGAACTGCTTAGCAGCATGAAGTCTCACAGCG | |
| | GACGGCAACTGGAACGGCGACCGCTCC | | | TGCCCGAGGAAA7CGACATCGCCGACACCGTGCTGAACGACGACGATATCGGC | |
| | ACATACGGCCTGTCTATCATCCTGCCC | | | GACTCATGCCACGAGGGCTTCCTGCTGAATGCCATCAGCAGCCACCTGCAGAC | |
| | CAGACCGAGCTGTCTTTTTACCTGCCT | | | ATGCGGCTGTTCTGTGGTGGTGGGCTCAAGCGCCGAGAAGGTGAACAAGATCG | |
| | CTGCACAGAGTGTGCGTGGACAGACTG | | | TGCGGACCCTGTGCCTGTTCCTGACACCTGCTGAGCGGAAGTGCAGCAGACTG | |
| | ACCCACATCATCCGGAAGGGCAGAATC | | | TGTGAAGCCGAATCCAGCTTTAAGTACGAGTCTGGCCTCTTCGTGCAAGGCCT | |
| | TGGATGCACAAGGAACGGCAGGAGAAC | | | GCTGAAGGACAGCACCGGCTCTTTTGTGCTGCCTTTTAGACAGGTGATGTACG | |
| | GTGCAGAAAATCATCCTGGAAGGAACA | | | CCCCTTACCCCACCACACACATCGACGTTGATGTCAACACCGTGAAACAGATG | |
| | GAGCGGATGGAAGATCAGGGCCAGAGC | | | CCTCCATGTCACGAGCACATCTACAACCAGAGAAGATACATGAGAAGCGAGCT | |
| | ATCATACCCATGCTGACTGGCGAGGTG | | | GACCGCCTTTTGGCGGGCCACCAGCGAGGAAGATATGGCCCAGGACACCATCA | |
| | ATCCCTGTGATGGAACTGCTGTCAAGC | | | TCTATACCGACGAGTCCTTCACCCCTGATCTGAACATCTTCCAAGACGTGCTG | |
| | ATGAAAAGCCACTCTGTCCCCGAGGAA | | | CACCGGGACACACTGGTCAAGGCCTTCCTGGACCAAGTGTTCCAGCTGAAGCC | |
| | ATCGACATCGCTGATACCGTGCTCAAC | | | CGGCCTGAGCCTGCGGAGCACCTTCCTGGCTCAGTTCCTGCTGGTGCTTCACC | |
| | GACGACGATATCGGCGATAGCTGCCAC | | | GGAAGGCCCTGACCCTTATCAAGTACATCGAGGACGACACCCAGAAGGGCAAA | |
| | GAGGGCTTCCTGCTGAACGCCATCAGC | | | AAGCCTTTCAAGAGCCTGAGAAATCTGAAAATCGACCTGGATCTGACAGCCGA | |
| | AGCCACCTGCAGACATGCGGCTGCAGC | | | AGGCGATCTGAACATCATCATGGCCCTTGCTGAGAAAATCAAGCCAGGCCTGC | |
| | GTCGTGGTGGGCTCTAGCGCCGAAAAG | | | ACAGCTTTATCTTCGGCAGACCTTTCTACACCAGCGTGCAGGAGAGAGATGTG | |
| | GTGAACAAGATCGTGCGGACCCTGTGT | | | CTGATGACCTTCTGA (SEQ ID NO: 48) | |
| | CTGTTCTTGACCCCTGCTGAAAGAAAG | | | | |
| | TGCAGCAGACTGTGCGAGGCCGAGAGC | | | | |
| | AGCTTCAAGTACGAGTCTGGCCTGTTT | | | | |
| | GTGCAGGGCCTGCTGAAAGACAGCACA | | | | |
| | GGCAGCTTCGTGCTGCCCTTCAGACAG | | | | |
| | GTGATGTACGCCCCTTACCCTACCACC | | | | |
| | CACATTGACGTGGACGTGAACACCGTG | | | | |
| | AAGCAGATGCCTCCGTGCCACGAGCAC | | | | |
| | ATCTACAACCAGCGTAGATACATGAGA | | | | |
| | TCCGAGCTGACAGCTTTCTGGCGGGCC | | | | |
| | ACCTCTGAAGAGGATATGGCCCAGGAC | | | | |
| | ACCATCATCTATACCGACGAGAGCTTC | | | | |
| | ACCCCTGATCTGAATATCTTCCAAGAC | | | | |
| | GTGCTGCATAGAGACACCCTGGTGAAA | | | | |
| | GCCTTCCTGGATCAAGTGTTCCAGCTG | | | | |
| | AAGCCTGGACTGAGCCTGCGGAGCACC | | | | |
| | TTCCTGGCCCAGTTCCTGCTCGTGCTT | | | | |
| | CATAGAAAGGCCCTGACACTGATCAAG | | | | |
| | TACATCGAGGACGACACACAGAAGGGC | | | | |
| | AAAAAGCCCTTCAAGAGCCTGAGAAAC | | | | |
| | CTGAAGATCGACCTGGACCTGACCGCC | | | | |
| | GAGGGCGATCTGAACATCATCATGGCT | | | | |
| | CTGGCCGAGAAGATCAAGCCCGGCCTG | | | | |
| | CACAGCTTTATCTTTGGCAGACCTTTC | | | | |
| | TACACCAGCGTGCAAGAGAGAGATGTG | | | | |
| | CTGATGACCTTTTGA (SEQ ID | | | | |
| | NO: 95) | | | | |
| gene 37 | ATGTCTACCCTGTGTCCTCCTCCAAGC | 56.09% | AscI | ATGAGCACCCTCTGTCCTCCTCCATCTCCTGCCGTGGCAAAGACCGAGATCGC | 55.93% |
| | CCCGCCGTGGCCAAGACTGAGATCGCC | | [GGCGCGCC]; | CCTGTCCGGCAAAAGCCCCCTGCTGGCCGCTACATTCGCCTACTGGGACAACA | |
| | CTGAGCGGCAAATCTCCTCTGCTCGCT | | NotI | TCCTCGGACCTAGAGTGCGGCACATCTGGGCCCCTAAGACCGAGCAGGTTCTG | |
| | GCTACCTTCGCCTACTGGGACAACATC | | [GCGGCCGC] | CTGAGCGACGGCGAGATAACATTTCTGGCCAACCACACCCTGAACGGCGAGAT | |
| | CTGGGACCTAGAGTGCGGCACATCTGG | | | CCTGAGAAACGCCGAGAGCGGCGCCATCGATGTGAAGTTCTTCGTGCTCTCTG | |
| | GCCCCTAAGACCGAGCAGGTCCTGCTG | | | AGAAGGGCGTGATCATTGTGTCCCTGATCTTCGACGGCAACTGGAACGGCGAT | |
| | AGCGACGGAGAGATAACATTTCTGGCC | | | AGATCCACCTACGGCCTGAGCATCATCCTGCCCCAGACAGAGCTGTCTTTTTA | |
| | AACCACACACTGAACGGCGAGATCCTC | | | CCTGCCTCTGCACCGGGTGTGCGTGGACAGACTGACACACATCATCAGAAAGG | |
| | AGAAATGCCGAGAGCGGCGCCATCGAC | | | GCAGAATCTGGATGCACAAGGAACGGCAGGAGAACGTGCAGAAAATCATCCTG | |
| | GTGAAGTTCTTCGTGCTGTCTGAGAAG | | | GAAGGCACCGAGAGAATGGAAGATCAGGGCCAGAGCATCATTCCTATGCTGAC | |
| | GGCGTGATCATTGTGTCCCTGATCTTC | | | TGGAGAGGTGATCCCCGTGATGGAACTGCTGTCTAGCATGAAAAGCCACAGCG | |
| | GACGGCAACTGGAACGGCGACAGAAGC | | | TGCCCGAGGAAATCGACATCGCCGACACCGTGCTGAACGACGACGACATCGGC | |
| | ACCTACGGCCTGAGCATCATCCTGCCT | | | GACAGCTGCCACGAGGGCTTCCTGCTCAATGCCATCAGCTCCCACCTGCAGAC | |
| | CAGACAGAGCTGTCCTTTTACCTGCCA | | | ATGCGGCTGCAGCGTGGTCGTGGGCAGCAGCGCCGAAAAGGTGAACAAGATCG | |
| | CTGCACCGGGTGTGCGTGGATAGACTG | | | TGCGGACACTGTGTCTGTTCCTGACCCCTGCTGAAAGAAAGTGCAGCAGACTG | |
| | ACACACATCATTAGAAAGGGCAGAATC | | | TGCGAGGCCGAATCTAGCTTTAAGTACGAGAGCGGCCTCTTCGTGCAAGGCCT | |
| | TGGATGCACAAGGAAAGACAGGAGAAC | | | GCTGAAGGACTCCACAGGCAGCTTCGTGCTGCCTTTTAGACAGGTGATGTACG | |
| | GTGCAGAAAATCATCCTGGAAGGTACA | | | CCCCTTATCCTACAACCCACATCGACGTGGACGTCAATACCGTGAAGCAGATG | |
| | GAGCGGATGGAAGATCAGGGCCAGAGC | | | CCTCCATGTCACGAGCACATCTACAACCAGAGAAGATACATGAGAAGCGAGCT | |
| | ATCATCCCTATGCTGACCGGCGAGGTG | | | GACCGCTTTTTGGCGGGCCACAAGCGAGGAAGATATGGCCCAGGACACCATCA | |
| | ATCCCCGTTATGGAACTCCTGTCTTCT | | | TCTATACTGATGAGTCTTTCACCCCTGATCTGAACATCTTCCAAGATGTGCTC | |
| | ATGAAAAGCCACAGCGTCCCCGAGGAA | | | CATAGAGATACCCTGGTCAAAGCCTTCCTGGACCAGGTGTTCCAGCTGAAACC | |
| | ATCGACATCGCAGATACAGTGCTGAAC | | | CGGCCTGAGCCTGAGATCTACCTTCCTGGCTCAGTTCCTGCTGGTGCTGCACA | |
| | GACGACGATATAGGAGATAGCTGTCAC | | | GAAAGGCCCTGACCCTGATCAAGTACATCGAGGATGATACCCAGAAGGGAAAA | |
| | GAGGGCTTCCTGTTAAACGCCATCAGC | | | AAGCCCTTCAAGTCCCTGCGGAACCTGAAGATCGACCTGGATCTGACCGCCGA | |
| | AGCCACCTGCAGACCTGTGGCTGCAGC | | | GGGCGACCTGAATATCATCATGGCCCTGGCCGAAAAGATCAAGCCAGGACTGC | |
| | GTGGTGGTCGGCTCTAGCGCCGAAAAG | | | ATAGCTTCATCTTCGGCAGACCTTTCTACACATCTGTGCAGGAGCGGGACGTG | |
| | GTGAACAAGATCGTGCGGACCCTGTGC | | | CTGATGACCTTCTGA (SEQ ID NO: 49) | |
| | CTGTTCCTGACACCTGCTGAACGGAAG | | | | |
| | TGCAGCAGACTGTGCGAGGCCGAGAGC | | | | |
| | AGTTTTAAGTACGAGTCCGGCCTGTTC | | | | |
| | GTGCAAGGCCTGCTGAAGGACTCTACA | | | | |
| | GGCAGCTTCGTGCTGCCTTTCAGACAG | | | | |
| | GTGATGTACGCCCCTTACCCCACCACC | | | | |
| | CACATCGACGTGGACGTGAACACCGTG | | | | |
| | AAGCAGATGCCTCCGTGCCACGAGCAC | | | | |
| | ATCTACAACCAGCGGAGATACATGCGG | | | | |
| | AGCGAGCTGACCGCTTTCTGGCGGGCC | | | | |
| | ACCAGCGAAGAGGACATGGCTCAGGAC | | | | |
| | ACCATCATCTATACAGACGAGAGCTTC | | | | |
| | ACCCCTGACCTGAATATCTTTCAAGAC | | | | |
| | GTGCTGCACAGAGATACCCTCGTGAAA | | | | |
| | GCCTTCCTGGACCAGGTGTTCCAGCTG | | | | |
| | AAACCTGGACTGTCACTGAGAAGCACC | | | | |
| | TTTCTGGCCCAGTTCCTGCTGGTCCTG | | | | |
| | CACAGAAAGGCCCTGACCCTTATCAAG | | | | |
| | TACATCGAGGATGACACCCAGAAGGGC | | | | |
| | AAGAAGCCCTTCAAGAGCCTGAGAAAC | | | | |
| | CTGAAGATCGACCTGGATCTGACAGCC | | | | |
| | GAAGGCGACCTGAACATCATCATGGCC | | | | |
| | CTGGCCGAAAAGATTAAGCCTGGCCTG | | | | |
| | CATTCTTTCATCTTCGGCCGCCCCTTC | | | | |
| | TACACCAGCGTGCAGGAGAGAGATGTG | | | | |
| | CTGATGACCTTCTGA (SEQ ID | | | | |
| | NO: 96) | | | | |
| gene 38 | ATGAGCACCCTGTGTCCTCCTCCTAGC | 55.88% | AscI | ATGAGCACACTCTGTCCTCCTCCGAGCCCAGCCGTGGCAAAGACCGAGATCGC | 56.27% |
| | CCTGCCGTGGCAAAGACCGAGATCGCC | | [GGCGCGCC]; | CCTGTCTGGCAAGTCCCCTCTGCTGGCCGCCACCTTCGCCTACTGGGACAACA | |
| | CTGAGCGGGAAGTCACCCCTGCTGGCC | | NotI | TCCTGGGCCCTAGAGTGCGGCACATCTGGGCCCCTAAGACCGAGCAGGTGCTG | |
| | GCTACATTTGCCTACTGGGACAACATC | | [GCGGCCGC] | CTGAGCGACGGAGAAATCACCTTCCTGGCTAATCACACCCTGAACGGCGAGAT | |
| | CTGGGCCCTAGAGTGCGGCACATCTGG | | | CCTGCGGAACGCCGAAAGCGGCGCCATCGACGTGAAGTTCTTCGTGCTGAGCG | |
| | GCCCCTAAGACCGAGCAGGTGCTGCTC | | | AGAAGGGAGTGATCATCGTGTCCCTGATCTTCGACGGCAACTGGAACGGCGAC | |
| | AGTGATGGCGAGATAACATTCCTCGCC | | | CGATCTACATACGGCCTGAGCATCATCCTGCCACAGACAGAGCTGAGCTTTTA | |
| | AACCACACACTGAATGGCGAAATCCTT | | | CCTGCCCCTGCATAGAGTGTGCGTGGACAGACTGACCCACATCATTAGAAAGG | |
| | AGAAATGCCGAGAGCGGTGCTATCGAC | | | GCAGAATCTGGATGCACAAGGAACGGCAGGAGAACGTGCAAAAGATCATCCTG | |
| | GTAAAGTTCTTCGTGCTGTCTGAAAAG | | | GAAGGCACCGAAAGAATGGAAGATCAGGGCCAGAGCATCATTCCTATGCTGAC | |
| | GGCGTGATCATCGTGTCCCTGATCTTC | | | CGGCGAGGTGATCCCCGTGATGGAACTGTTGTCCAGCATGAAATCTCACAGCG | |
| | GACGGCAACTGGAACGGCGATAGAAGC | | | TCCCCGAGGAAATCGACATCGCCGACACCGTGCTGAACGACGACGATATCGGC | |
| | ACCTACGGCCTGAGCATCATCCTGCCT | | | GACTCATGCCATGAGGGATTCCTGCTGAATGCCATCAGCAGCCACCTGCAGAC | |
| | CAGACAGAGCTGAGCTTCTATCTGCCT | | | CTGCGGCTGTAGCGTGGTCGTGGGCAGCAGTGCCGAGAAGGTGAACAAGATCG | |
| | CTGCACAGGGTGTGCGTGGACAGACTG | | | TGCGGACCCTGTGTCTGTTTCTGACCCCTGCCGAAAGAAAGTGCAGCAGACTG | |
| | ACTCACATTATTAGAAAAGGCAGAATC | | | TGCGAGGCCGAGAGCAGCTTCAAGTACGAGTCTGGCCTGTTCGTGCAGGGCCT | |
| | TGGATGCACAAGGAAAGACAGGAGAAC | | | GCTGAAAGACAGCACCGGATCTTTCGTGCTGCCTTTTAGACAGGTGATGTACG | |
| | GTGCAAAAGATCATCCTGGAAGGCACC | | | CCCCTTATCCTACAACCCACATTGACGTCGACGTCAACACCGTGAAACAGATG | |
| | GAGAGAATGGAAGATCAGGGCCAGAGC | | | CCTCCGTGCCACGAGCACATCTACAACCAGAGGCGGTACATGAGATCTGAGCT | |
| | ATCATCCCTATGCTGACCGGCGAGGTG | | | GACAGCCTTCTGGCGGGCCACAAGCGAAGAGGACATGGCCCAGGACACCATCA | |
| | ATCCCCGTGATGGAACTGCTGAGTTCT | | | TCTACACTGATGAGAGCTTCACCCCTGATCTGAACATCTTCCAAGACGTGCTG | |
| | ATGAAGAGTCACTCTGTGCCCGAGGAA | | | CACCGGGACACCCTGGTCAAGGCCTTTCTCGACCAGGTGTTCCAGCTGAAGCC | |
| | ATCGACATCGCCGACACAGTGCTGAAC | | | CGGCCTGTCCCTGAGATCCACATTTCTTGCTCAGTTCCTGCTGGTGCTGCACA | |
| | GACGACGATATCGGCGACTCCTGCCAC | | | GAAAAGCCCTGACACTGATCAAGTACATCGAGGACGACACACAGAAGGGCAAA | |
| | GAGGGCTTCCTGCTGAACGCCATCAGC | | | AAGCCTTTCAAAAGCCTGAGAAACCTGAAGATCGATCTGGACCTGACCGCCGA | |
| | AGCCACCTGCAGACCTGCGGCTGCAGC | | | GGGCGATCTTAATATCATCATGGCCCTGGCCGAAAAAATCAAGCCTGGCCTGC | |
| | GTGGTGGTCGGCAGCTCCGCCGAAAAG | | | ACTCTTTTATCTTCGGCAGACCTTTCTACACCAGCGTGCAGGAGAGAGATGTG | |
| | GTGAACAAGATCGTGCGGACCCTGTGC | | | CTGATGACCTTCTGA (SEQ ID NO: 50) | |
| | CTGTTCCTGACGCCCGCCGAAAGAAAG | | | | |
| | TGCAGTAGACTGTGCGAGGCCGAAAGC | | | | |
| | TCTTTCAAGTACGAGAGCGGCCTGTTT | | | | |
| | GTGCAGGGCCTGCTCAAGGACAGCACT | | | | |
| | GGATCTTTCGTGCTCCCCTTCAGACAG | | | | |
| | GTGATGTACGCCCCTTACCCTACAACA | | | | |
| | CACATCGATGTGGACGTGAACACCGTG | | | | |
| | AAGCAGATGCCTCCATGTCACGAGCAC | | | | |
| | ATCTACAACCAGCGTAGATACATGAGA | | | | |
| | AGCGAGCTGACAGCCTTTTGGCGGGCC | | | | |
| | ACAAGCGAGGAAGATATGGCCCAGGAC | | | | |
| | ACCATCATCTACACCGACGAGAGCTTC | | | | |
| | ACCCCTGACCTGAATATCTTTCAGGAC | | | | |
| | GTTCTGCACCGGGACACCCTTGTGAAG | | | | |
| | GCCTTCCTGGACCAGGTTTTCCAGCTG | | | | |
| | AAACCTGGCCTCTCCCTGCGGAGCACA | | | | |
| | TTCCTGGCTCAGTTCCTGCTGGTGCTG | | | | |
| | CATAGAAAGGCCCTGACACTGATCAAG | | | | |
| | TACATCGAGGATGACACCCAGAAGGGC | | | | |
| | AAAAAGCCTTTTAAGAGCCTGAGAAAC | | | | |
| | CTGAAGATCGACCTGGATCTGACCGCC | | | | |
| | GAGGGCGACCTGAACATCATCATGGCT | | | | |
| | CTGGCCGAGAAAATCAAGCCCGGACTG | | | | |
| | CATAGCTTCATCTTCGGAAGACCTTTC | | | | |
| | TACACCAGCGTGCAGGAGCGGGACGTG | | | | |
| | CTGATGACCTTCTGA (SEQ ID | | | | |
| | NO: 97) | | | | |
| gene 39 | ATGAGCACACTGTGCCCCCCCCCGAGC | 56.43% | AscI | ATGAGCACCCTCTGCCCCCCCCCCAGCCCCGCCGTGGCCAAGACAGAAATCGC | 56.83% |
| | CCGGCCGTGGCCAAGACAGAGATCGCC | | [GGCGCGCC]; | CCTGTCTGGCAAGTCCCCTCTGCTGGCCGCCACCTTTGCCTACTGGGACAACA | |
| | CTGAGCGGCAAGTCCCCTCTGCTGGCC | | NotI | TCCTGGGCCCTAGAGTGCGGCACATCTGGGCCCCTAAGACCGAGCAAGTGCTG | |
| | GCCACCTTCGCCTACTGGGACAACATC | | [GCGGCCGC] | CTGTCTGATGGAGAAATCACCTTCCTGGCTAATCACACACTGAACGGCGAGAT | |
| | CTGGGCCCTAGAGTGCGGCACATCTGG | | | CCTGCGGAACGCCGAGTCTGGAGCCATCGACGTGAAATTCTTCGTGCTGAGCG | |
| | GCCCCTAAGACCGAGCAGGTTCTGCTG | | | AGAAGGGCGTGATCATCGTGTCCCTGATCTTCGACGGCAACTGGAACGGCGAT | |
| | AGTGATGGCGAGATAACATTCCTGGCC | | | AGAAGCACCTACGGCCTGTCCATCATCCTGCCTCAGACAGAGCTGTCCTTCTA | |
| | AACCACACCCTGAACGGCGAGATCCTG | | | CCTGCCACTGCACCGGGTGTGCGTGGACAGACTGACCCACATTATTAGAAAGG | |
| | AGAAATGCCGAATCTGGCGCCATCGAC | | | GCAGAATCTGGATGCACAAGGAACGGCAGGAGAACGTGCAGAAGATCATTCTG | |
| | GTGAAGTTCTTCGTGCTGTCTGAGAAG | | | GAAGGGACCGAGAGAATGGAAGATCAGGGCCAGAGCATCATCCCTATGCTGAC | |
| | GGCGTGATCATTGTGTCCCTGATCTTC | | | TGGCGAGGTGATCCCCGTGATGGAACTGCTGAGCTCCATGAAAAGCCATTCTG | |
| | GACGGCAACTGGAACGGCGATAGAAGC | | | TCCCCGAGGAAATCGACATCGCCGACACCGTGCTGAACGACGACGATATCGGC | |
| | ACCTACGGCCTGAGCATCATCCTGCCA | | | GACAGCTGCCACGAGGGCTTCCTGCTGAATGCCATCAGCTCTCATCTGCAGAC | |
| | CAGACCGAACTGTCGTTCTACCTGCCT | | | CTGCGGCTGCAGCGTCGTGGTGGGCTCTAGCGCCGAGAAGGTGAACAAGATCG | |
| | CTGCACCGAGTGTGCGTGGACAGACTG | | | TGCGGACACTGTGCCTGTTCCTGACACCTGCCGAGAGGAAGTGCAGCAGACTG | |
| | ACCCACATCATCAGAAAGGGAAGAATC | | | TGTGAAGCCGAATCTAGCTTTAAGTACGAGAGCGGCCTGTTCGTGCAAGGCCT | |
| | TGGATGCACAAGGAAAGACAGGAGAAC | | | GCTGAAGGACAGCACAGGCAGCTTCGTGCTGCCTTTCAGACAGGTGATGTACG | |
| | GTGCAGAAGATCATCCTGGAAGGTACA | | | CCCCTTACCCCACCACCCACATCGATGTTGACGTGAACACCGTGAAGCAGATG | |
| | GAACGGATGGAAGATCAGGGACAGAGC | | | CCTCCATGTCACGAGCACATCTACAACCAGCGGAGATACATGCGGAGCGAGCT | |
| | ATCATCCCCATGCTGACAGGCGAAGTG | | | GACCGCCTTTTGGCGGGCCACAAGCGAAGAGGACATGGCTCAGGACACAATCA | |
| | ATCCCTGTGATGGAACTGCTGAGCTCT | | | TCTACACTGATGAGAGCTTCACCCCTGATCTGAACATTTTCCAAGACGTGCTC | |
| | ATGAAAAGCCACAGCGTGCCTGAGGAA | | | CACAGAGATACCCTGGTGAAGGCCTTCCTGGACCAGGTTTTCCAGCTGAAACC | |
| | ATCGACATCGCTGATACCGTGCTGAAC | | | TGGACTGAGCCTGAGAAGCACCTTCCTGGCCCAGTTCCTGCTCGTGCTGCACA | |
| | GACGACGATATCGGCGACAGCTGCCAC | | | GAAAGGCCCTGACCCTTATCAAGTATATCGAGGACGACACCCAGAAAGGCAAA | |
| | GAGGGCTTCCTGCTGAACGCCATCAGC | | | AAGCCCTTCAAGAGCCTGAGAAACCTGAAGATCGACCTGGATCTGACCGCCGA | |
| | AGTCACCTGCAGACATGCGGCTGTAGC | | | GGGAGATCTGAACATCATCATGGCCCTGGCCGAGAAAATCAAGCCTGGCCTGC | |
| | GTCGTGGTGGGCTCCAGCGCCGAGAAA | | | ACAGCTTTATCTTCGGCCGCCCCTTTTACACAAGCGTGCAGGAGAGAGACGTG | |
| | GTGAACAAGATCGTGCGCACCCTGTGC | | | CTGATGACCTTCTGA (SEQ ID NO: 51) | |
| | CTGTTCCTGACCCCTGCTGAGCGGAAA | | | | |
| | TGCAGCAGACTGTGTGAAGCCGAGAGC | | | | |
| | TCCTTTAAGTACGAGAGCGGCCTTTTT | | | | |
| | GTGCAGGGCCTGCTGAAGGACAGCACA | | | | |
| | GGCAGCTTCGTGCTGCCCTTCCGGCAG | | | | |
| | GTGATGTACGCCCCTTATCCTACCACC | | | | |
| | CACATCGACGTCGACGTGAACACCGTG | | | | |
| | AAGCAGATGCCTCCTTGCCACGAGCAC | | | | |
| | ATCTACAACCAGAGAAGATACATGAGA | | | | |
| | TCCGAGCTGACCGCCTTCTGGCGGGCC | | | | |
| | ACAAGCGAGGAAGATATGGCCCAAGAC | | | | |
| | ACCATCATCTACACTGATGAGAGTTTC | | | | |
| | ACCCCTGATCTGAACATCTTTCAGGAC | | | | |
| | GTGCTCCATCGGGACACCCTGGTGAAA | | | | |
| | GCTTTCCTGGATCAAGTCTTTCAGCTG | | | | |
| | AAGCCCGGCCTGTCCCTGCGGTCCACC | | | | |
| | TTCCTGGCCCAGTTCCTGCTCGTGCTG | | | | |
| | CACCGGAAGGCCCTGACCCTGATCAAA | | | | |
| | TACATCGAGGACGACACACAGAAAGGC | | | | |
| | AAAAAGCCTTTCAAGAGCCTGAGAAAC | | | | |
| | CTGAAAATCGATCTGGACCTGACAGCC | | | | |
| | GAGGGCGACCTGAATATCATCATGGCC | | | | |
| | CTGGCTGAAAAGATTAAGCCCGGACTG | | | | |
| | CATTCTTTCATCTTCGGCAGACCTTTC | | | | |
| | TACACCAGCGTGCAGGAGAGAGATGTC | | | | |
| | CTCATGACCTTTTGA (SEQ ID | | | | |
| | NO: 98) | | | | |
| gene 40 | ATGAGCACATTGTGTCCTCCACCATCT | 56.36% | AscI | ATGAGCACACTGTGTCCTCCTCCTAGCCCCGCCGTGGCCAAGACCGAGATCGC | 55.99% |
| | CCTGCCGTGGCCAAGACCGAAATCGCC | | [GGCGCGCC]; | CCTCAGCGGCAAGTCTCCACTGCTCGCCGCTACCTTCGCCTACTGGGACAACA | |
| | CTGAGCGGCAAGAGCCCCCTGCTCGCC | | NotI | TCCTGGGCCCTAGAGTGCGGCACATCTGGGCCCCTAAGACAGAGCAGGTCCTT | |
| | GCCACCTTCGCCTACTGGGACAACATC | | [GCGGCCGC] | CTGAGCGACGGCGAGATAACATTCCTGGCCAACCACACACTGAACGGCGAGAT | |
| | CTGGGCCCTAGAGTGCGGCACATCTGG | | | CCTCAGGAACGCCGAATCTGGCGCCATCGACGTGAAGTTCTTCGTGCTGTCTG | |
| | GCCCCTAAGACCGAGCAGGTTCTGCTG | | | AGAAGGGCGTGATTATTGTGTCCCTGATCTTCGACGGAAATTGGAACGGCGAC | |
| | AGCGACGGCGAGATAACATTCCTGGCT | | | CGGAGCACATACGGCCTGTCCATCATCCTGCCCCAGACGGAACTGTCTTTTTA | |
| | AATCACACCCTGAATGGCGAGATCCTG | | | CCTGCCTCTGCACAGAGTGTGCGTGGACAGACTGACCCACATCATTAGAAAGG | |
| | CGGAACGCCGAAAGCGGAGCCATCGAC | | | GCAGAATCTGGATGCACAAGGAAAGACAGGAGAACGTGCAGAAAATCATCCTG | |
| | GTGAAGTTCTTCGTGCTGAGCGAGAAG | | | GAAGGTACAGAGAGAATGGAAGATCAGGGACAGAGCATCATCCCTATGCTGAC | |
| | GGAGTGATCATCGTGTCCCTGATCTTC | | | TGGCGAAGTGATCCCCGTGATGGAACTGCTGTCCAGCATGAAAAGCCACAGCG | |
| | GACGGCAACTGGAACGGCGACCGCTCC | | | TGCCCGAGGAAATCGACATCGCCGACACTGTGCTGAACGACGATGATATCGGC | |
| | ACCTACGGCCTGTCTATCATCCTGCCT | | | GACAGCTGCCATGAGGGCTTCCTGCTGAATGCCATCAGCTCTCACCTGCAGAC | |
| | CAGACCGAGCTGAGTTTCTACCTGCCT | | | CTGTGGATGTAGCGTGGTGGTCGGCAGCAGCGCCGAAAAGGTGAACAAGATTG | |
| | CTGCACCGGGTGTGCGTGGACAGACTG | | | TGCGGACCCTGTGCCTGTTCCTCACACCTGCTGAGAGAAAGTGCAGCAGACTG | |
| | ACACACATCATCCGGAAAGGCAGAATC | | | TGCGAGGCCGAGAGCAGCTTCAAGTACGAGAGCGGCCTGTTCGTGCAGGGCCT | |
| | TGGATGCACAAGGAACGGCAGGAGAAC | | | GCTGAAGGACAGCACCGGCTCCTTCGTTCTGCCTTTCCGGCAGGTGATGTACG | |
| | GTGCAAAAGATCATCCTGGAAGGCACC | | | CCCCTTACCCCACCACCCACATCGATGTTGACGTGAATACCGTGAAACAGATG | |
| | GAGAGAATGGAAGATCAGGGCCAGAGC | | | CCTCCATGTCACGAGCACATCTACAACCAGAGAAGATACATGAGAAGCGAGCT | |
| | ATCATTCCCATGCTGACTGGAGAAGTG | | | GACCGCCTTCTGGCGGGCCACCAGCGAAGAGGACATGGCCCAGGACACCATCA | |
| | ATCCCTGTGATGGAACTGCTGAGCAGC | | | TCTACACCGACGAGAGCTTCACCCCTGATCTGAACATCTTTCAGGATGTGCTC | |
| | ATGAAGTCCCACAGCGTGCCCGAGGAA | | | CATAGAGATACCCTGGTCAAGGCCTTCCTGGACCAGGTGTTCCAGCTGAAACC | |
| | ATCGACATCGCCGACACCGTGCTGAAC | | | TGGACTGAGCCTGCGCAGCACCTTCCTGGCTCAATTTCTACTTGTGCTGCACC | |
| | GACGATGACATAGGAGATTCATGCCAC | | | GGAAGGCCCTGACACTGATCAAGTACATCGAGGACGACACCCAGAAGGGCAAA | |
| | GAGGGCTTCCTGCTGAACGCCATCAGC | | | AAGCCCTTTAAGAGCCTGAGAAACCTGAAGATCGACCTGGATCTGACAGCCGA | |
| | TCTCACCTGCAGACATGCGGCTGTAGC | | | AGGCGATCTGAACATCATCATGGCTCTTGCTGAGAAAATCAAGCCAGGACTGC | |
| | GTCGTGGTGGGCTCTAGCGCCGAAAAG | | | ATTCTTTCATCTTCGGCCGCCCCTTCTACACATCTGTGCAGGAGCGGGACGTG | |
| | GTGAACAAGATCGTCAGAACCCTGTGC | | | CTGATGACCTTCTGA (SEQ ID NO: 52) | |
| | CTGTTCCTGACCCCTGCTGAAAGAAAG | | | | |
| | TGCAGCCGGCTGTGCGAGGCCGAGTCC | | | | |
| | AGTTTTAAGTACGAGAGCGGCTTGTTT | | | | |
| | GTGCAGGGACTGCTGAAGGACAGCACC | | | | |
| | GGCAGCTTCGTGCTCCCCTTCAGACAG | | | | |
| | GTGATGTACGCCCCTTATCCTACAACC | | | | |
| | CACATTGATGTGGATGTTAACACCGTG | | | | |
| | AAGCAGATGCCTCCATGTCATGAGCAC | | | | |
| | ATCTACAACCAGCGTAGATACATGCGG | | | | |
| | AGCGAGCTGACCGCCTTTTGGCGGGCC | | | | |
| | ACAAGCGAGGAAGATATGGCCCAGGAT | | | | |
| | ACCATCATCTACACAGACGAGAGCTTC | | | | |
| | ACCCCTGATCTGAATATCTTCCAAGAC | | | | |
| | GTCCTGCACAGAGACACCCTCGTGAAG | | | | |
| | GCCTTCCTGGACCAGGTGTTCCAGCTG | | | | |
| | AAACCCGGCCTGAGCCTGAGAAGCACC | | | | |
| | TTCCTCGCTCAGTTCCTGCTGGTGCTG | | | | |
| | CATAGAAAGGCCCTGACCCTGATCAAG | | | | |
| | TACATCGAGGACGACACACAGAAAGGA | | | | |
| | AAAAAGCCCTTCAAGAGCCTGAGAAAC | | | | |
| | CTGAAGATCGACCTGGATCTGACAGCC | | | | |
| | GAGGGCGATCTGAACATCATCATGGCT | | | | |
| | CTGGCCGAGAAGATCAAGCCTGGCCTC | | | | |
| | CACTCCTTCATCTTCGGCAGACCTTTT | | | | |
| | TACACCAGCGTGCAAGAGCGGGACGTG | | | | |
| | CTCATGACCTTTTGA (SEQ ID | | | | |
| | NO: 99) | | | | |

According to some embodiments, the codon optimized sequence comprises SEQ ID NO: 14, shown below.

SEQ ID NO: 100
ATGAGCACCCTGTGTCCTCCACCTAGCCCCGCCGTGGCCAAGACAGAGAT
CGCCCTGAGCGGAAAAAGCCCTCTGCTGGCCGCTACATTTGCCTACTGGG
ACAACATCCTGGGCCCTAGAGTGCGGCACATTTGGGCCCCTAAGACCGAA
CAGGTGCTGCTGAGTGATGGAGAGATCACCTTCCTGGCTAATCACACCCT
TAACGGCGAAATCCTGCGGAACGCCGAGAGCGGAGCCATCGACGTGAAGT
TCTTCGTGTTAAGCGAGAAGGGCGTGATCATTGTGTCCCTGATCTTCGAC
GGCAACTGGAACGGCGATAGATCTACATACGGCCTGTCCATCATTCTTCC
ACAGACAGAGCTGTCTTTCTACCTGCCTCTGCACCGGGTGTGCGTGGACA
GACTGACCCACATTATTAGAAAAGGCAGAATCTGGATGCACAAGGAACGG
CAGGAGAACGTGCAAAAGATCATCCTCGAGGGTACAGAGAGAATGGAAGA
TCAGGGCCAGAGCATCATCCCCATGCTGACCGGCGAGGTGATCCCTGTGA
TGGAACTGCTGAGCAGCATGAAAAGCCACTCTGTCCCCGAGGAAATCGAC
ATCGCCGACACCGTGCTGAACGACGATGATATAGGAGATTCATGCCACGA
GGGCTTCCTGCTGAATGCCATCAGCTCTCACCTGCAGACCTGTGGCTGCA
GCGTCGTGGTGGGCAGCAGCGCCGAGAAAGTGAACAAGATCGTGCGGACC
CTGTGCCTGTTCCTGACCCCTGCTGAAAGAAAGTGCAGCAGACTGTGTGA
AGCCGAATCTAGCTTTAAGTACGAGTCTGGACTGTTTGTGCAGGGCCTGC
TGAAGGACAGCACAGGCTCCTTCGTGCTGCCCTTCAGACAGGTTATGTAC
GCCCCTTACCCCACCACCCACATCGATGTGGACGTCAACACAGTGAAGCA
GATGCCTCCTTGCCACGAGCACATCTACAACCAGCGTAGATACATGCGGA
GCGAGCTGACCGCCTTTTGGCGGGCCACCTCTGAAGAGGACATGGCCCAG
GATACAATCATCTATACCGACGAGTCCTTCACCCCTGATCTGAATATCTT
CCAAGACGTGCTTCATAGAGATACACTGGTGAAAGCCTTCCTCGACCAGG
TGTTCCAGCTGAAGCCTGGCCTGAGCCTGAGGTCCACATTCCTCGCTCAG
TTCCTGCTCGTGCTGCACAGAAAGGCCCTGACCCTTATCAAGTACATCGA
GGATGACACCCAGAAGGGCAAGAAGCCGTTCAAGTCCCTCAGAAACCTGA
AAATCGACCTGGACCTGACAGCCGAGGGAGATCTGAACATCATCATGGCT
CTGGCCGAAAAGATCAAGCCCGGCCTGCATTCTTTCATCTTCGGCAGACC
TTTTTACACCAGCGTGCAAGAGCGGGACGTGCTGATGACATTCTGA.

According to some embodiments, the codon optimized sequence is at least 85%, at least 90%, at least 95%, at least 96%, at least 97%, at least 98% or at least 99% identical to SEQ ID NO: 100.

According to some embodiments, the codon optimized sequence comprises SEQ ID NO: 15, shown below.

SEQ ID NO: 101
ATGAGCACCCTGTGCCCTCCACCTAGCCCCGCCGTGGCCAAGACAGAGAT
CGCCCTTTCTGGCAAGTCCCCACTGCTGGCCGCTACCTTCGCCTATTGGG
ACAACATCTTGGGCCCCAGAGTGCGGCACATCTGGGCCCCTAAGACCGAG
CAGGTGCTGCTGAGTGATGGCGAGATCACCTTCCTGGCTAATCACACCCT
GAACGGCGAGATCCTGAGAAACGCCGAGAGCGGCGCCATCGACGTGAAAT
TCTTCGTGCTGAGCGAGAAAGGCGTGATCATCGTGTCCCTGATCTTCGAC
GGAAATTGGAACGGCGACAGAAGCACCTACGGCCTGAGCATCATCCTCCC
CCAGACCGAGCTGTCCTTCTACCTGCCTCTGCATAGAGTGTGCGTGGACC
GCCTGACACACATCATTAGAAAGGGCAGAATCTGGATGCACAAGGAACGG
CAGGAGAACGTGCAGAAAATTATCCTGGAAGGTACAGAGAGAATGGAAGA
TCAGGGACAGTCTATCATCCCCATGCTGACCGGCGAAGTGATCCCTGTGA
TGGAACTGCTGTCTAGCATGAAGTCTCATTCTGTGCCTGAGGAAATCGAC
ATCGCCGACACCGTGCTGAACGACGACGACATCGGCGATAGCTGCCACGA
GGGCTTCCTGCTGAACGCCATTAGCAGCCACCTGCAGACCTGCGGATGTA
GCGTGGTGGTCGGCAGCAGCGCCGAGAAGGTGAACAAGATCGTGCGGACA
CTGTGCCTGTTCCTCACACCTGCTGAAAGAAAGTGCAGCAGACTGTGTGA
AGCCGAAAGCAGCTTTAAGTACGAGAGCGGCCTGTTCGTGCAAGGCCTGC
TGAAGGACAGCACAGGCTCTTTTGTGCTGCCTTTCAGACAGGTGATGTAC
GCCCCTTACCCCACCACACACATTGACGTGGACGTGAACACCGTGAAGCA
GATGCCTCCTTGTCACGAGCACATCTACAACCAGAGAAGATACATGAGAT
CTGAGCTGACCGCCTTTTGGCGGGCCACCAGCGAAGAGGACATGGCCCAG
GATACCATCATCTACACTGATGAGAGCTTCACCCCTGATCTGAACATTTT
CCAGGACGTGCTGCACAGAGATACCCTGGTGAAGGCCTTCCTGGACCAGG
TCTTTCAGCTGAAACCTGGACTGAGCCTGCGGTCCACATTCCTGGCCCAA
TTTCTGCTGGTGCTGCACCGGAAGGCTCTGACTCTGATCAAGTATATCGA
GGACGATACACAGAAGGGCAAAAAGCCCTTCAAGAGCCTGAGAAATCTGA
AGATCGATCTGGATCTGACAGCCGAGGGCGACCTGAATATCATCATGGCC
CTGGCAGAAAAGATTAAGCCTGGCCTGCACAGCTTCATCTTCGGCCGTCC
ATTCTACACCTCTGTGCAGGAGCGGGACGTTCTCATGACCTTCTGA

According to some embodiments, the codon optimized sequence is at least 85%, at least 90%, at least 95%, at least 96%, at least 97%, at least 98% or at least 99% identical to SEQ ID NO: 101.

According to some embodiments, the codon optimized sequence comprises SEQ ID NO: 16, shown below.

SEQ ID NO: 102
ATGAGCACCCTTTGTCCTCCTCCATCTCCTGCCGTGGCCAAGACAGAAAT
CGCCCTGTCCGGCAAGTCCCCTCTGCTGGCTGCTACATTTGCCTACTGGG
ACAACATCCTGGGACCTAGAGTTAGACACATCTGGGCCCCTAAGACCGAG
CAGGTTCTGCTGAGTGATGGCGAGATAACATTCCTGGCCAACCACACCCT
GAATGGAGAAATCCTGAGAAACGCCGAGAGCGGCGCCATCGATGTGAAGT
TCTTCGTGCTGAGCGAGAAGGGCGTGATCATTGTGTCCCTGATCTTCGAC
GGCAACTGGAACGGCGATAGATCTACATACGGCCTGTCCATCATCCTGCC
CCAGACCGAGCTGAGCTTTTACCTGCCTCTGCACAGAGTTTGTGTGGACA
GACTGACTCACATTATCAGAAAGGGAAGAATCTGGATGCACAAGGAAAGA
CAGGAGAACGTGCAGAAGATTATTCTGGAAGGTACAGAGAGAATGGAAGA
TCAGGGCCAGAGCATCATCCCCATGCTGACCGGCGAGGTGATCCCTGTGA
TGGAACTGCTGAGCAGCATGAAAAGCCACAGCGTGCCCGAGGAAATCGAC
ATCGCCGACACAGTGCTGAATGATGACGACATCGGCGACAGCTGCCACGA
GGGCTTCCTGCTGAACGCTATCAGCTCTCATCTGCAGACATGCGGCTGTA
GCGTCGTGGTGGGCAGCTCCGCCGAGAAGGTGAACAAGATCGTGCGGACA
CTGTGCCTGTTCCTCACCCCTGCTGAACGGAAATGCTCTAGACTCTGCGA
GGCCGAGAGCAGCTTCAAGTACGAGTCCGGCCTCTTCGTGCAAGGCCTGC
TGAAAGACAGTACAGGCAGCTTCGTGCTGCCTTTCAGACAGGTCATGTAC
GCCCCTTACCCCACCACCCACATCGATGTGGACGTGAACACCGTGAAGCA
GATGCCTCCGTGCCACGAGCACATCTACAACCAGAGAAGATACATGCGGT
CTGAACTGACAGCCTTTTGGCGGGCCACCAGCGAAGAGGACATGGCCCAG
GACACCATCATCTACACCGACGAGTCTTTCACCCCTGACCTGAATATCTT
TCAGGATGTGCTGCACAGAGATACCCTGGTCAAGGCCTTCCTGGACCAGG
TGTTCCAGCTGAAGCCTGGACTGTCTCTGCGGAGCACCTTCCTGGCCCAA
TTTCTTCTGGTGCTCCACCGGAAGGCCCTGACACTGATCAAGTACATCGA
GGACGACACCCAGAAAGGAAAAAAGCCGTTCAAGTCCCTGCGGAACCTGA
AGATCGACCTGGATCTGACCGCCGAGGGCGACCTGAACATCATCATGGCC
CTGGCTGAGAAAATCAAGCCTGGCCTGCACAGCTTCATCTTCGGCAGACC
TTTCTACACCAGCGTGCAGGAGCGGGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 102.

According to some embodiments, the codon optimized sequence comprises SEQ ID NO: 17, shown below.

SEQ ID NO: 103
ATGAGCACACTGTGCCCCCCACCTTCTCCAGCCGTGGCCAAGACCGAGAT
CGCCCTTTCTGGCAAGAGCCCTCTGCTGGCCGCCACATTCGCCTACTGGG
ACAACATCCTGGGCCCTAGAGTGCGGCACATCTGGGCCCCTAAGACCGAG
CAGGTGCTGCTGAGTGATGGCGAAATAACATTCCTGGCTAATCACACCCT
CAACGGAGAGATCCTGAGAAATGCCGAGAGCGGCGCCATCGACGTCAAGT
TCTTCGTGCTGTCTGAAAAGGGCGTGATCATAGTTTCTCTGATCTTCGAC
GGCAACTGGAACGGCGACAGAAGCACCTACGGCCTGTCCATCATCCTGCC
CCAGACAGAACTGAGCTTTTACCTGCCTCTGCACAGAGTGTGCGTGGACC
GGCTGACCCACATCATTAGAAAGGGCAGAATCTGGATGCACAAGGAACGG
CAGGAGAACGTGCAGAAGATCATCCTGGAAGGGACCGAAAGAATGGAAGA
TCAGGGCCAGAGCATCATTCCTATGCTGACAGGCGAGGTGATCCCCGTGA
TGGAACTGCTGAGCAGCATGAAGTCTCACTCTGTCCCCGAGGAAATCGAC
ATCGCCGACACTGTGCTCAACGACGACGATATCGGCGATAGCTGCCACGA
GGGATTTCTGCTGAACGCCATTTCTAGCCACCTGCAGACCTGTGGCTGCA
GCGTGGTCGTGGGCAGCTCCGCCGAGAAGGTGAACAAGATCGTGCGGACC
CTGTGCCTGTTTCTGACACCTGCTGAACGGAAGTGCAGTAGACTGTGTGA
AGCCGAGAGCAGCTTCAAATACGAGAGCGGACTGTTCGTTCAAGGCCTGC
TGAAGGACAGCACCGGAAGCTTCGTGCTGCCTTTCAGACAGGTGATGTAC
GCCCCTTACCCCACAACACACATTGATGTCGATGTGAACACAGTGAAACA
GATGCCTCCATGTCACGAGCACATCTACAACCAGAGGCGGTACATGAGAA
GCGAGCTGACCGCCTTTTGGCGGGCCACCAGCGAGGAAGATATGGCCCAG
GACACAATCATCTACACTGATGAGTCCTTTACCCCTGATCTGAATATCTT
CCAGGACGTGCTGCATAGAGACACCCTGGTGAAGGCCTTCCTGGACCAGG
TGTTCCAGCTGAAGCCTGGACTCAGCCTGCGGAGCACCTTCCTCGCTCAG
TTCCTGCTCGTGCTGCACAGAAAGGCCCTGACCCTGATCAAGTACATCGA
GGACGACACCCAGAAAGGCAAAAAGCCCTTCAAGTCCCTCAGAAACCTGA
AAATCGACCTGGACCTGACCGCCGAAGGCGACCTGAACATCATCATGGCC
CTGGCCGAGAAGATCAAACCTGGCCTGCACAGCTTCATCTTCGGCAGACC
TTTCTACACCAGCGTGCAGGAGAGAGATGTGCTGATGACCTTTTGA

According to some embodiments, the codon optimized sequence is at least 85%, at least 90%, at least 95%, at least 96%, at least 97%, at least 98% or at least 99% identical to SEQ ID NO: 103.

According to some embodiments, the codon optimized sequence comprises SEQ ID NO: 18, shown below.

SEQ ID NO: 104
ATGAGCACCCTGTGCCCTCCACCTAGCCCTGCCGTGGCCAAGACAGAGAT
CGCACTGTCCGGCAAGTCCCCACTGCTGGCCGCCACCTTCGCCTACTGGG
ACAACATCCTGGGCCCTAGAGTGCGGCACATTTGGGCCCCTAAGACCGAG
CAGGTGCTGCTGTCTGATGGCGAGATCACCTTCCTGGCTAATCACACCCT
GAACGGCGAAATCCTGAGAAATGCCGAGAGCGGCGCCATCGACGTGAAGT
TCTTCGTGCTGTCTGAGAAGGGCGTGATCATCGTGTCCCTGATCTTCGAC
GGCAACTGGAACGGCGACCGGAGCACCTACGGCCTGAGCATCATCCTGCC
TCAGACCGAACTGTCCTTTTACCTGCCTCTGCACAGAGTGTGCGTGGACA
GACTGACACACATCATCAGAAAGGGCAGAATCTGGATGCACAAGGAAAGA
CAGGAGAACGTGCAGAAGATCATTCTGGAAGGTACAGAAAGAATGGAAGA
TCAGGGCCAGAGCATCATTCCTATGCTGACCGGCGAGGTGATCCCCGTGA
TGGAACTGCTGAGCAGCATGAAAAGCCACAGCGTCCCCGAGGAAATCGAC
ATCGCTGATACCGTGCTGAACGACGACGATATCGGCGATAGCTGCCACGA
GGGCTTCCTGCTGAACGCCATCAGCAGCCACCTGCAGACCTGCGGCTGCA
GCGTGGTCGTGGGCAGCTCCGCCGAGAAGGTGAACAAGATCGTGCGGACC
CTGTGTCTGTTCCTGACCCCTGCTGAGAGAAAGTGCAGCAGACTGTGTGA
AGCCGAGTCCTCCTTCAAATACGAGAGCGGATTGTTTGTGCAAGGACTCC
TGAAGGACAGCACAGGCTCTTTCGTGCTGCCCTTCAGACAGGTGATGTAC
GCCCCTTACCCCACCACACACATTGACGTGGACGTCAACACAGTGAAACA
GATGCCTCCATGTCACGAGCACATCTACAACCAGAGACGGTACATGAGAA
GCGAGCTGACCGCCTTTTGGCGGGCCACAAGCGAGGAAGATATGGCCCAA
GATACAATCATCTATACAGACGAGTCTTTCACCCCTGATCTGAATATCTT
TCAGGACGTCCTGCACCGGGACACCCTGGTGAAGGCCTTCCTGGATCAGG
TGTTCCAGCTGAAACCCGGCCTGTCTCTGCGGTCCACCTTCCTGGCCCAG
TTCCTGCTGGTCCTGCATAGAAAAGCCCTGACCCTGATCAAGTACATCGA
GGACGACACGCAGAAAGGAAAGAAGCCCTTCAAGAGCCTTAGAAACCTGA
AGATCGACCTGGACCTCACAGCCGAAGGCGACCTGAACATCATCATGGCT
CTGGCCGAAAAAATCAAGCCTGGCCTGCATAGCTTCATCTTCGGCAGACC
TTTCTACACCTCTGTCCAGGAGAGAGATGTGCTGATGACATTCTGA

According to some embodiments, the codon optimized sequence is at least 85%, at least 90%, at least 95%, at least 96%, at least 97%, at least 98% or at least 99% identical to SEQ ID NO: 104.

According to some embodiments, the codon optimized sequence comprises SEQ ID NO: 19, shown below.

SEQ ID NO: 105
ATGAGCACCCTCTGTCCTCCCCCCAGCCCTGCTGTGGCCAAGACAGAGAT
CGCCCTGTCTGGAAAGTCCCCTCTGCTGGCTGCTACATTCGCCTACTGGG
ACAACATCCTGGGCCCCAGAGTGCGGCACATCTGGGCCCCTAAGACCGAG
CAGGTGCTCCTGAGCGACGGCGAGATCACCTTCCTGGCTAATCACACCCT
GAACGGCGAGATCCTGAGAAATGCCGAAAGCGGCGCCATCGACGTGAAGT
TCTTCGTGCTGTCTGAGAAGGGCGTGATCATTGTGTCCCTGATCTTCGAC
GGCAACTGGAACGGCGATAGATCTACATACGGCCTGAGCATCATCCTGCC
TCAGACCGAGCTGTCCTTCTACCTGCCTCTGCACAGAGTGTGCGTGGACA
GACTGACACACATCATTAGAAAGGGCAGGATCTGGATGCACAAGGAAAGA
CAGGAGAACGTGCAGAAGATCATCCTGGAAGGGACCGAAAGAATGGAAGA
TCAGGGCCAGAGCATCATCCCTATGCTGACCGGCGAAGTGATCCCCGTGA
TGGAACTGCTGAGTTCCATGAAAAGCCACTCTGTGCCCGAGGAAATCGAC
ATCGCCGACACCGTGCTGAACGACGACGACATAGGAGATAGCTGCCATGA
GGGCTTCCTGCTGAACGCCATCAGCAGCCACCTGCAGACCTGCGGTTGTA
GCGTGGTGGTGGGCTCTAGCGCCGAGAAGGTGAACAAGATCGTGCGGACC
CTGTGCCTGTTCCTGACACCTGCCGAACGAAAATGCTCTAGACTGTGTGA
AGCCGAGAGCAGCTTTAAGTACGAGAGCGGCCTGTTCGTGCAAGGCCTGC
TTAAAGACAGCACCGGCAGCTTCGTTCTGCCATTCAGACAGGTGATGTAC
GCCCCTTACCCTACCACCCACATTGACGTCGACGTGAACACCGTGAAACA
GATGCCTCCTTGCCACGAGCACATCTACAACCAGAGAAGATACATGCGGA
GCGAGTTGACCGCCTTCTGGCGGGCCACCAGCGAGGAAGATATGGCCCAG
GACACCATCATCTACACCGACGAGAGCTTCACCCCTGACCTGAACATCTT
TCAGGATGTGCTGCATAGAGATACACTGGTGAAGGCCTTTCTCGACCAGG
TTTTCCAGCTGAAGCCCGGCCTGAGCCTGCGGAGCACATTTCTGGCTCAA
TTTCTCCTGGTCCTGCACCGGAAAGCCCTGACACTGATCAAGTACATCGA
GGATGACACCCAGAAAGGCAAAAAGCCCTTCAAGAGCCTGAGAAACCTGA
AGATCGACCTGGACCTGACCGCCGAGGGCGACCTTAATATCATCATGGCC
CTGGCTGAAAAGATTAAGCCTGGCCTGCACAGCTTCATCTTCGGCAGACC
TTTCTATACAAGCGTGCAGGAGCGGGACGTGCTGATGACATTCTGA

According to some embodiments, the codon optimized sequence is at least 85%, at least 90%, at least 95%, at least 96%, at least 97%, at least 98% or at least 99% identical to SEQ ID NO: 105.

According to some embodiments, the codon optimized sequence comprises SEQ ID NO: 20, shown below.

SEQ ID NO: 106
ATGAGCACACTGTGTCCTCCACCATCTCCTGCCGTGGCCAAGACCGAGAT
CGCCCTGAGCGGAAAAAGCCCCCTGCTGGCCGCTACCTTCGCCTACTGGG
ACAACATCCTGGGCCCTAGAGTGCGGCACATCTGGGCCCCTAAGACAGAG
CAGGTGCTCCTGAGTGATGGCGAGATAACATTCCTGGCTAATCACACCCT
GAATGGCGAAATCCTGAGAAACGCCGAAAGTGGCGCCATTGACGTGAAGT
TCTTCGTGCTGTCCGAGAAGGGCGTGATCATCGTGTCCCTGATCTTCGAC
GGCAACTGGAACGGCGATAGAAGCACCTACGGCCTGTCTATCATCCTGCC
TCAGACCGAGCTGAGCTTCTACCTGCCTCTGCACAGAGTGTGCGTGGACA
GACTGACACACATCATTAGAAAGGGCAGAATCTGGATGCACAAGGAACGG
CAGGAGAACGTGCAGAAAATCATCCTGGAAGGGACCGAAAGGATGGAAGA
TCAGGGCCAGAGCATCATCCCCATGCTGACTGGAGAGGTGATCCCTGTTA
TGGAACTGCTGAGCAGCATGAAGAGCCACAGCGTGCCCGAAGAGATTGAC
ATCGCCGACACCGTGCTGAACGACGACGACATAGGAGATTCATGCCACGA
AGGATTCCTGCTCAACGCCATCAGCAGCCACCTGCAGACATGCGGCTGCT
CTGTGGTCGTGGGCAGCAGCGCCGAGAAAGTGAACAAGATCGTGCGGACC
CTCTGTCTGTTTCTCACACCCGCTGAGCGGAAGTGCAGCAGACTGTGCGA
GGCCGAGTCTAGCTTTAAGTACGAGAGCGGCCTGTTCGTGCAAGGCCTGC
TGAAGGACTCTACCGGCTCCTTTGTGCTCCCTTTTAGACAGGTGATGTAC
GCCCCTTACCCCACCACCCACATTGATGTGGACGTCAACACCGTGAAACA
GATGCCTCCTTGCCACGAGCACATCTACAACCAGAGACGGTACATGCGGA
GCGAGCTGACCGCCTTCTGGCGGGCCACCTCCGAGGAAGATATGGCCCAG
GACACCATCATCTATACTGATGAGTCTTTCACCCCTGATCTGAACATCTT
TCAGGATGTGCTGCACCGGGACACCCTGGTGAAGGCTTTCCTCGACCAGG
TGTTCCAGCTGAAACCTGGCCTCAGCCTCAGAAGCACATTCCTGGCCCAG
TTCCTGCTCGTGCTCCATAGAAAGGCCCTGACACTGATCAAGTACATCGA
GGATGATACACAGAAGGGCAAGAAGCCTTTCAAGTCCCTGCGGAACCTGA
AGATCGACCTGGACCTGACAGCCGAAGGCGACCTGAACATCATTATGGCC
CTGGCCGAGAAGATCAAGCCCGGCCTGCATTCTTTCATCTTCGGCAGACC
TTTCTACACCAGCGTGCAGGAGAGAGATGTTCTGATGACCTTCTGA

According to some embodiments, the codon optimized sequence is at least 85%, at least 90%, at least 95%, at least 96%, at least 97%, at least 98% or at least 99% identical to SEQ ID NO: 106.

According to some embodiments, the codon optimized sequence comprises SEQ ID NO: 21, shown below.

SEQ ID NO: 21
ATGAGCACACTGTGTCCTCCACCGAGCCCTGCCGTGGCCAAGACAGAGAT
CGCCCTGAGCGGCAAGTCCCCTCTGCTGGCCGCCACATTCGCCTACTGGG
ACAACATCCTGGGACCTAGAGTTAGACACATTTGGGCCCCTAAGACCGAG
CAGGTGCTGCTGAGTGATGGAGAGATCACCTTCCTGGCCAACCACACCCT
GAACGGCGAGATCCTGAGAAATGCCGAGAGCGGCGCTATCGATGTGAAGT
TCTTCGTGCTGTCTGAGAAGGGTGTTATCATCGTGTCCCTGATCTTCGAC
GGCAACTGGAACGGCGATAGAAGCACCTACGGCCTGAGCATCATCCTGCC
TCAGACCGAGCTGAGCTTCTACCTGCCACTGCACAGAGTGTGCGTGGACA
GACTGACACACATCATTAGAAAGGGAAGAATCTGGATGCACAAGGAAAGA
CAGGAGAACGTGCAAAAGATCATCCTGGAAGGTACAGAGCGGATGGAAGA
TCAGGGCCAGAGCATCATACCCATGCTGACAGGCGAAGTGATCCCCGTGA
TGGAACTCCTCAGCTCCATGAAAAGCCACAGCGTGCCCGAGGAAATCGAC
ATCGCCGACACCGTGCTGAATGACGACGACATCGGCGACAGCTGCCACGA
AGGCTTCCTGCTGAACGCCATCAGCAGCCACCTGCAGACATGCGGCTGCA
GCGTCGTGGTGGGCTCTTCTGCCGAGAAGGTGAACAAGATCGTGCGGACC
CTGTGCCTGTTCCTGACACCTGCTGAGAGGAAGTGCAGCAGACTGTGTGA
AGCCGAATCCAGCTTTAAGTACGAGTCTGGCCTGTTTGTGCAAGGCCTCC
TGAAAGACTCCACCGGCAGCTTTGTGCTGCCTTTTAGACAGGTGATGTAC
GCCCCTTACCCCACCACCCACATCGACGTCGACGTGAACACCGTGAAGCA
GATGCCTCCGTGCCACGAGCACATCTACAACCAGCGGAGATACATGAGAA
GCGAGCTGACCGCCTTCTGGCGGGCCACCAGCGAGGAAGATATGGCACAG
GACACCATCATCTACACCGACGAGAGCTTCACCCCTGACCTGAACATCTT
CCAAGATGTGCTGCACCGGGACACCCTGGTGAAAGCCTTCCTGGATCAGG
TCTTTCAGCTGAAACCCGGCCTGTCTCTGAGATCTACCTTCCTGGCCCAG
TTCCTGCTTGTGCTGCATAGAAAGGCCCTGACGCTGATCAAGTACATCGA
GGATGATACACAGAAAGGAAAAAAGCCCTTCAAGAGCCTGCGGAACCTGA
AGATCGACCTGGACCTGACTGCCGAGGGCGACCTGAACATCATCATGGCC
CTGGCTGAAAAGATTAAGCCAGGCCTGCACTCCTTCATCTTTGGCAGACC
TTTCTACACCTCCGTGCAGGAGAGAGATGTGCTGATGACCTTCTGA

According to some embodiments, the codon optimized sequence is at least 85%, at least 90%, at least 95%, at least 96%, at least 97%, at least 98% or at least 99% identical to SEQ ID NO: 21.

According to some embodiments, the codon optimized sequence comprises SEQ ID NO: 22, shown below.

SEQ ID NO: 22
ATGAGCACACTCTGTCCTCCCCCCAGCCCCGCCGTGGCCAAGACCGAGAT
CGCCCTGAGCGGAAAGTCCCCTCTGCTTGCTGCTACATTTGCCTACTGGG
ACAACATCTTGGGCCCTAGAGTGCGGCACATCTGGGCCCCTAAGACCGAG
CAGGTCCTGCTGAGTGATGGCGAAATCACCTTCCTGGCTAATCACACCCT
GAACGGCGAGATCCTGAGAAACGCCGAGTCCGGCGCCATCGATGTGAAGT
TCTTCGTGCTGTCTGAAAAGGGCGTGATCATTGTGTCCCTGATCTTCGAC
GGAAATTGGAACGGCGATAGATCTACCTACGGCCTGTCTATCATCCTGCC
TCAGACAGAGCTGAGCTTCTACCTGCCCCTGCACAGAGTGTGCGTGGACC
GGCTGACACACATTATCAGAAAGGGCAGAATCTGGATGCACAAGGAACGC
CAGGAGAACGTGCAGAAGATCATCCTGGAAGGCACCGAGAGAATGGAAGA
TCAGGGCCAGAGCATCATCCCCATGCTGACCGGCGAGGTGATTCCTGTGA
TGGAACTGCTGAGCAGCATGAAAAGCCACTCCGTCCCCGAGGAAATCGAC
ATCGCAGATACCGTGCTGAACGACGATGACATCGGCGACAGCTGCCACGA
GGGATTCCTCCTGAATGCCATCAGCTCTCACCTGCAGACATGCGGCTGTA
GCGTCGTCGTGGGCAGCAGCGCCGAGAAAGTGAACAAGATCGTGCGGACA
CTGTGTCTGTTCCTCACACCTGCCGAAAGAAAGTGCAGCAGACTGTGCGA
GGCCGAGTCTAGCTTCAAGTACGAGAGCGGCCTCTTCGTGCAGGGACTGC
TGAAGGACAGCACCGGCTCTTTCGTGCTGCCTTTCAGACAGGTGATGTAC
GCCCCTTACCCCACCACCCACATCGACGTTGACGTGAACACCGTGAAACA
GATGCCCCCGTGCCATGAACACATCTACAACCAGCGGAGATACATGAGAA
GCGAGCTGACCGCCTTCTGGCGGGCCACCAGCGAGGAAGATATGGCTCAG
GATACCATCATCTATACAGACGAGAGCTTCACCCCTGACCTGAACATCTT
TCAGGACGTGCTGCATAGAGATACACTCGTGAAGGCCTTTCTGGATCAGG
TTTTCCAGCTGAAGCCTGGCCTGAGCCTGAGATCCACCTTCCTGGCACAA
TTTCTGCTGGTGCTGCACCGGAAGGCCCTGACCCTGATCAAGTACATCGA
GGACGACACACAGAAAGGCAAGAAGCCCTTTAAGAGCCTGCGGAACCTGA
AAATTGATCTGGACCTGACTGCCGAGGGCGACCTGAATATCATCATGGCC
CTGGCCGAGAAGATCAAGCCTGGACTGCACTCTTTCATCTTCGGCAGACC
TTTCTACACAAGCGTGCAAGAGCGGGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 22.

According to some embodiments, the codon optimized sequence comprises SEQ ID NO: 23, shown below.

SEQ ID NO: 23
ATGAGCACCCTGTGTCCTCCGCCCAGCCCTGCCGTGGCCAAGACCGAAAT
CGCCCTGAGCGGAAAAAGCCCCCTGCTGGCCGCCACCTTTGCCTACTGGG
ACAACATCCTGGGCCCTAGAGTGCGGCACATCTGGGCCCCTAAGACCGAG
CAGGTGCTGCTGAGCGACGGCGAGATAACATTCCTCGCTAATCACACACT
GAACGGCGAAATCCTGAGAAATGCCGAAAGCGGCGCCATCGACGTTAAGT
TCTTCGTGCTGTCTGAAAAGGGCGTGATCATCGTGTCCCTGATCTTCGAC
GGCAACTGGAACGGCGATAGATCAACCTACGGCCTGAGCATCATCCTGCC
TCAGACCGAGCTGTCTTTCTACCTGCCTCTGCATAGAGTGTGCGTGGACA
GACTGACACACATCATCAGAAAGGGAAGAATCTGGATGCACAAGGAAAGA
CAGGAGAACGTGCAGAAGATCATTCTGGAAGGTACAGAGAGAATGGAAGA
TCAGGGACAGAGCATCATTCCTATGCTGACTGGAGAGGTGATCCCCGTGA
TGGAACTGCTGAGCTCCATGAAAAGCCACTCTGTTCCTGAGGAAATCGAC
ATCGCCGACACCGTGCTGAACGACGACGATATTGGAGATAGCTGCCACGA
GGGCTTCCTTCTGAACGCCATCAGCAGCCACCTGCAGACATGCGGCTGCA
GCGTCGTGGTGGGCTCCAGCGCCGAGAAGGTGAACAAGATCGTGCGGACC
CTGTGCCTGTTCCTGACCCCTGCTGAGCGGAAGTGCAGTAGACTGTGTGA
AGCCGAGAGCAGCTTCAAGTACGAGTCCGGCCTGTTTGTGCAGGGCCTGC
TGAAGGACAGCACAGGCAGCTTCGTGCTGCCCTTCAGACAAGTGATGTAC
GCCCCTTACCCCACCACCCACATCGACGTCGACGTGAACACCGTGAAGCA
GATGCCTCCATGTCACGAGCACATCTACAACCAGAGGCGGTACATGAGAT
CTGAGCTGACCGCCTTTTGGCGGGCCACAAGCGAGGAAGATATGGCCCAG
GACACCATCATCTACACCGACGAGTCTTTCACCCCTGATCTGAATATCTT
TCAGGATGTCCTGCACCGGGACACACTGGTGAAGGCCTTCCTGGACCAGG
TGTTCCAGCTGAAGCCCGGCCTGTCCCTGCGGAGCACCTTCCTGGCCCAA
TTTCTGCTCGTGCTTCACAGAAAGGCCCTGACACTGATCAAGTACATCGA
GGACGACACCCAGAAAGGCAAGAAGCCTTTCAAGTCCCTGCGCAACCTGA
AAATCGATCTGGACCTGACCGCCGAGGGCGACCTGAACATCATCATGGCC
CTTGCCGAGAAAATCAAACCTGGCCTGCACAGCTTCATCTTCGGCAGACC
TTTTTATACCAGCGTGCAGGAGAGAGATGTGCTTATGACCTTCTGA

According to some embodiments, the codon optimized sequence is at least 85%, at least 90%, at least 95%, at least 96%, at least 97%, at least 98% or at least 99% identical to SEQ ID NO: 23.

According to some embodiments, the codon optimized sequence comprises SEQ ID NO: 24, shown below.

SEQ ID NO: 24
ATGAGCACCCTGTGTCCTCCACCATCTCCTGCCGTGGCCAAGACAGAGAT
CGCCCTGTCTGGCAAGTCACCTCTGCTGGCCGCTACATTCGCCTACTGGG
ACAACATCCTTGGACCTAGAGTGCGGCACATCTGGGCCCCTAAGACCGAG
CAGGTTCTGCTGAGCGACGGCGAGATAACATTTCTGGCCAACCACACACT
TAATGGCGAGATCCTGAGAAACGCCGAGTCTGGCGCCATCGATGTGAAGT
TCTTCGTGCTGTCCGAGAAGGGCGTGATCATCGTGTCCCTGATCTTCGAC
GGCAACTGGAACGGCGACCGGTCTACCTACGGCCTGTCCATCATCCTGCC
CCAGACAGAGCTGAGTTTCTACCTGCCACTGCATAGAGTGTGCGTGGACA
GACTGACACACATCATCAGAAAGGGCAGAATCTGGATGCACAAGGAACGG
CAGGAGAACGTGCAGAAGATCATCCTCGAGGGCACCGAGCGGATGGAAGA
TCAGGGCCAGAGCATCATTCCTATGCTGACAGGCGAAGTGATCCCCGTGA
TGGAACTGCTGTCTAGCATGAAAAGCCACAGCGTGCCGGAAGAGATCGAC
ATCGCCGACACAGTGCTGAACGACGACGACATCGGCGATAGCTGCCACGA
GGGCTTCCTCCTGAACGCCATCAGCTCCCACCTGCAGACCTGCGGCTGCT
CTGTGGTCGTGGGCTCTAGCGCCGAAAAGGTGAACAAGATCGTGCGGACC
CTGTGCCTGTTCCTGACACCTGCTGAAAGAAAATGCAGCAGACTGTGTGA
AGCCGAGAGCAGCTTCAAGTACGAGAGCGGCCTGTTCGTGCAGGGACTCC
TGAAGGACAGCACAGGCAGCTTTGTGCTGCCTTTCAGACAGGTGATGTAC
GCCCCCTACCCCACCACCCACATCGACGTCGACGTGAACACCGTGAAACA
GATGCCTCCTTGTCACGAGCACATCTACAACCAGCGGAGATACATGAGAA
GCGAGCTGACGGCCTTTTGGCGGGCCACTTCCGAGGAAGATATGGCTCAG
GACACAATCATCTACACTGATGAGTCCTTCACCCCTGATCTGAATATCTT
TCAGGACGTGCTGCACAGAGATACCCTGGTGAAGGCCTTCCTGGATCAGG
TCTTTCAGCTGAAGCCCGGCCTGTCTCTGAGAAGCACCTTCCTGGCCCAG
TTCCTGCTTGTGCTGCACCGGAAGGCCCTGACCCTGATCAAGTACATCGA
GGACGATACCCAGAAAGGAAAAAAGCCTTTTAAGAGCCTGCGGAACCTGA
AAATCGACCTGGACCTGACCGCCGAGGGAGATCTGAACATCATCATGGCC
CTGGCTGAAAAGATTAAGCCTGGACTGCACAGCTTCATCTTCGGCAGACC
TTTCTACACCAGCGTGCAAGAGCGGGACGTGCTGATGACCTTTTGA

According to some embodiments, the codon optimized sequence is at least 85%, at least 90%, at least 95%, at least 96%, at least 97%, at least 98% or at least 99% identical to SEQ ID NO: 24.

According to some embodiments, the codon optimized sequence comprises SEQ ID NO: 25, shown below.

SEQ ID NO: 25
ATGAGCACACTGTGCCCTCCACCGAGCCCTGCTGTGGCCAAGACAGAGAT
CGCCCTCTCTGGCAAGAGCCCCCTGTTGGCCGCCACATTCGCCTACTGGG
ACAACATCCTGGGTCCTAGAGTGCGGCACATCTGGGCCCCTAAGACCGAG
CAGGTGCTGCTGAGTGATGGAGAAATAACATTCCTGGCCAACCACACCCT
GAACGGCGAAATCCTGAGAAACGCCGAGAGCGGTGCTATCGACGTGAAGT
TCTTCGTGCTCAGCGAGAAGGGAGTGATCATCGTGTCCCTGATCTTCGAC
GGCAACTGGAACGGCGACCGGAGCACCTACGGCCTGAGCATCATCCTGCC
TCAGACCGAGCTGAGCTTTTACCTGCCTCTGCACAGAGTGTGCGTGGACA
GACTGACCCACATCATTAGAAAGGGCAGAATCTGGATGCACAAGGAACGG
CAGGAGAACGTGCAGAAGATCATCCTCGAGGGTACAGAGAGAATGGAAGA
TCAGGGCCAGTCTATCATCCCTATGCTGACCGGCGAGGTGATCCCAGTGA
TGGAACTGCTGTCCAGCATGAAGAGTCACTCTGTTCCTGAGGAAATCGAC
ATCGCCGACACCGTGCTGAACGACGATGACATCGGCGATAGCTGCCACGA
GGGCTTCCTGCTGAATGCCATCAGCAGCCACCTGCAGACATGCGGCTGTA
GCGTGGTGGTCGGCAGCAGCGCCGAAAAAGTGAACAAGATCGTGCGGACC
CTCTGTCTGTTCCTGACACCTGCCGAGCGCAAGTGCAGCAGACTGTGTGA
AGCCGAATCCAGCTTCAAGTACGAGTCTGGACTCTTCGTGCAAGGCCTGC
TGAAGGACAGCACCGGCTCTTTTGTGCTGCCCTTCAGACAGGTCATGTAC
GCCCCATACCCCACCACACACATTGATGTTGACGTCAACACCGTGAAGCA
GATGCCTCCGTGCCATGAGCACATCTACAACCAGCGGAGATACATGAGAT
CTGAGCTGACCGCCTTTTGGCGGGCCACCAGCGAAGAGGATATGGCTCAA
GACACAATCATCTATACTGATGAGAGCTTCACCCCTGATCTGAATATCTT
TCAGGACGTGCTGCACCGAGACACCCTCGTGAAAGCCTTCCTGGACCAGG
TGTTCCAGCTGAAACCTGGCCTGTCTCTGAGAAGCACCTTCCTCGCCCAG
TTCCTGCTGGTGCTGCACAGAAAGGCCCTGACACTGATCAAGTACATCGA
GGACGACACCCAGAAAGGCAAGAAACCCTTTAAGTCCCTGCGGAATCTGA
AGATTGACCTGGATCTGACCGCCGAGGGCGACCTGAACATCATCATGGCC
CTGGCCGAGAAGATCAAGCCCGGCCTCCACAGCTTCATCTTTGGCAGACC
TTTCTACACCAGCGTGCAGGAGAGAGATGTGCTGATGACCTTCTGA

According to some embodiments, the codon optimized sequence is at least 85%, at least 90%, at least 95%, at least 96%, at least 97%, at least 98% or at least 99% identical to SEQ ID NO: 25.

According to some embodiments, the codon optimized sequence comprises SEQ ID NO: 26, shown below.

SEQ ID NO: 26
ATGAGCACCCTGTGTCCTCCACCGAGCCCTGCTGTGGCCAAGACCGAGAT
CGCCCTGAGCGGCAAATCTCCTCTGCTGGCCGCTACATTCGCCTACTGGG
ACAACATCCTGGGCCCTAGAGTGCGGCACATTTGGGCCCCTAAGACCGAG
CAGGTGCTGCTGAGCGACGGCGAAATCACCTTTCTGGCCAACCACACCCT
GAACGGCGAGATCCTGCGGAACGCCGAAAGCGGCGCCATCGACGTCAAGT
TCTTCGTGCTGTCTGAGAAGGGCGTGATCATTGTGTCCCTGATCTTCGAC
GGCAACTGGAACGGCGACAGAAGCACCTACGGCCTGTCCATCATACTGCC
CCAGACCGAGCTGTCTTTCTACCTGCCTCTGCACCGCGTGTGCGTGGATA
GACTGACCCACATCATTAGAAAAGGCAGAATCTGGATGCACAAGGAACGG
CAGGAGAACGTGCAGAAGATCATCCTGGAAGGGACCGAAAGAATGGAAGA
TCAGGGACAGAGCATCATCCCCATGCTGACTGGCGAGGTGATCCCTGTGA
TGGAACTGCTGAGCTCTATGAAAAGCCACAGCGTGCCCGAGGAAATCGAT
ATCGCTGATACCGTGCTGAACGACGATGACATCGGCGATAGCTGCCACGA
GGGCTTCCTGCTGAACGCCATCAGCAGCCACCTGCAGACATGCGGCTGTA
GCGTCGTGGTGGGCTCTTCCGCCGAGAAGGTGAACAAGATCGTGCGGACC
CTGTGCCTGTTCCTGACACCTGCCGAGAGAAAGTGCAGCAGACTGTGCGA
GGCCGAATCTTCTTTTAAGTACGAGAGCGGACTCTTCGTGCAAGGACTGC
TGAAAGACAGCACAGGCAGCTTTGTGCTGCCTTTCAGACAGGTTATGTAC
GCCCCCTACCCCACCACCCACATCGACGTGGACGTGAACACCGTGAAGCA
GATGCCTCCATGTCACGAGCACATCTACAACCAGCGGAGATACATGAGAT
CTGAACTGACCGCATTCTGGCGGGCCACCAGCGAAGAGGATATGGCCCAG
GACACAATCATCTATACAGACGAGAGCTTCACCCCTGATCTTAATATCTT
CCAAGACGTGCTGCACCGGGACACCCTGGTGAAAGCCTTCCTGGATCAAG
TGTTCCAGCTGAAGCCCGGCCTGAGCCTGAGATCCACATTCCTTGCTCAG
TTCCTGCTGGTCCTGCACAGAAAGGCCCTGACGCTGATCAAGTACATCGA
GGACGACACCCAGAAAGGCAAGAAGCCTTTCAAGAGCCTGAGAAACCTGA
AGATCGACCTGGACCTGACAGCCGAGGGCGACCTGAATATCATCATGGCC
CTGGCTGAAAAGATCAAGCCTGGACTGCATAGCTTCATCTTTGGAAGACC
TTTTTACACCTCCGTCCAAGAGCGGGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 26.

According to some embodiments, the codon optimized sequence comprises SEQ ID NO: 27, shown below.

SEQ ID NO: 27
ATGAGCACACTGTGCCCTCCTCCAAGCCCTGCCGTGGCCAAGACCGAGAT
AGCTCTGAGCGGCAAGAGCCCCCTGCTTGCCGCCACATTCGCCTACTGGG
ACAACATCCTGGGCCCCAGAGTGCGGCACATCTGGGCCCCTAAGACAGAG
CAGGTGCTGCTGAGCGACGGCGAGATCACCTTCCTGGCCAACCACACCCT
GAATGGCGAAATCCTGAGAAACGCCGAGAGCGGTGCTATCGATGTGAAGT
TCTTCGTGTTGTCTGAAAAGGGCGTGATCATAGTTTCTCTGATCTTTGAT
GGCAACTGGAACGGCGATAGATCCACATACGGCCTCTCCATCATACTCCC
CCAGACAGAGCTGAGCTTCTATCTGCCTCTGCACAGAGTGTGCGTGGACA
GACTGACCCACATCATTAGAAAGGGCAGAATCTGGATGCACAAGGAACGG
CAGGAGAACGTGCAAAAGATCATCCTGGAAGGTACAGAGCGGATGGAAGA
TCAGGGCCAGTCTATCATTCCTATGCTGACCGGCGAGGTGATCCCCGTGA
TGGAACTGCTGTCTAGCATGAAATCCCACAGCGTGCCGGAAGAAATCGAC
ATCGCCGACACCGTGCTGAACGACGATGACATAGGAGATAGCTGCCACGA
GGGCTTCCTGCTGAATGCCATCAGCAGCCACCTGCAGACCTGCGGCTGCA
GCGTGGTGGTCGGCAGCTCCGCCGAAAAGGTGAACAAGATCGTGCGGACC
CTCTGTCTGTTCCTGACCCCTGCTGAAAGAAAGTGCAGTAGACTGTGTGA
AGCCGAGAGCTCTTTTAAGTACGAGTCTGGACTTTTCGTGCAGGGCCTGC
TGAAGGACAGCACAGGCAGCTTCGTGCTGCCTTTTAGACAGGTGATGTAC
GCCCCTTACCCCACCACCCACATCGACGTGGACGTCAACACCGTGAAACA
GATGCCTCCTTGCCATGAGCACATCTACAACCAGAGACGGTACATGAGAA
GCGAGCTGACCGCCTTCTGGCGGGCCACCAGTGAAGAGGACATGGCACAG
GATACCATCATCTATACAGACGAGTCCTTCACCCCTGACCTGAACATCTT
CCAGGACGTGCTGCACAGAGATACCCTGGTCAAGGCTTTTCTGGACCAGG
TTTTCCAGCTGAAGCCTGGCCTGAGCCTGCGGTCCACCTTCCTGGCCCAG
TTCCTGCTGGTGCTGCACCGGAAGGCCCTGACCCTCATCAAGTACATCGA
GGACGACACCCAGAAAGGCAAAAAGCCTTTCAAGTCCCTGCGCAACCTGA
AAATTGACCTGGATCTGACAGCCGAGGGAGATCTGAATATCATCATGGCC
CTGGCCGAGAAGATCAAGCCCGGCCTGCATAGCTTCATCTTCGGCCGCCC
CTTTTACACCAGCGTGCAGGAGAGGGACGTGCTGATGACATTCTGA

According to some embodiments, the codon optimized sequence is at least 85%, at least 90%, at least 95%, at least 96%, at least 97%, at least 98% or at least 99% identical to SEQ ID NO: 27.

According to some embodiments, the codon optimized sequence comprises SEQ ID NO: 28, shown below.

SEQ ID NO: 28
ATGAGCACACTGTGTCCTCCACCTAGCCCTGCCGTGGCCAAGACCGAAAT
CGCCCTGAGCGGAAAGAGCCCCCTGCTGGCCGCCACCTTCGCCTACTGGG
ACAACATCCTGGGCCCTAGAGTGCGGCACATCTGGGCCCCTAAGACCGAG
CAGGTCTTGCTTTCTGATGGCGAAATCACCTTCCTCGCTAATCACACCCT
GAACGGCGAGATCCTGAGAAATGCCGAGTCCGGCGCCATTGACGTGAAGT
TCTTCGTGCTGAGCGAGAAGGGCGTGATCATCGTGTCCCTGATCTTCGAC
GGAAACTGGAACGGCGACAGAAGCACCTACGGCCTGTCCATCATCCTGCC
TCAGACCGAGCTGAGCTTCTACCTGCCACTGCATAGAGTGTGCGTGGACC
GGCTGACACACATCATCCGGAAGGGCAGAATCTGGATGCACAAGGAACGG
CAGGAGAACGTGCAGAAAATCATCCTGGAAGGTACAGAGAGAATGGAAGA
TCAGGGCCAGAGCATCATCCCTATGCTGACCGGCGAGGTGATCCCCGTGA
TGGAACTGCTCAGCTCTATGAAGTCCCACAGCGTGCCTGAGGAAATTGAC
ATCGCCGATACCGTGCTGAACGACGACGACATCGGCGACAGCTGCCACGA
GGGCTTCCTGCTGAACGCCATCAGCAGCCACCTGCAGACCTGCGGCTGCA
GCGTGGTGGTCGGCAGCTCCGCCGAGAAGGTGAACAAGATCGTGCGGACC
CTCTGTCTGTTCCTGACTCCTGCTGAAAGAAAGTGCAGTAGACTGTGCGA
GGCCGAATCTAGCTTCAAGTACGAGAGCGGCCTTTTTGTGCAGGGACTCC
TGAAGGACTCTACAGGCTCTTTCGTGCTGCCTTTTAGACAGGTGATGTAC
GCCCCCTACCCCACCACCCACATTGACGTGGATGTCAACACAGTGAAACA
GATGCCCCCCTGCCACGAGCACATCTACAACCAGAGGCGGTACATGCGGA
GCGAGCTGACCGCCTTCTGGCGGGCCACAAGCGAAGAGGACATGGCTCAA
GACACCATCATATATACAGACGAGAGCTTCACCCCTGATCTGAATATCTT
TCAGGACGTGCTGCACCGGGACACCCTGGTCAAGGCCTTTCTGGACCAGG
TGTTCCAGCTGAAACCTGGCCTGAGCCTGAGGTCCACCTTCTTGGCACAG
TTCCTGCTGGTGCTGCACAGAAAAGCCCTGACACTGATCAAATACATCGA
GGATGACACACAGAAGGGAAAAAAGCCCTTCAAGTCTCTGAGAAACCTGA
AGATCGATCTGGATCTGACAGCCGAGGGAGATCTGAACATCATCATGGCC
CTGGCTGAAAAGATCAAGCCTGGACTTCATTCTTTCATCTTCGGCAGACC
TTTCTACACCAGCGTGCAGGAGCGGGACGTTCTGATGACCTTTTGA

According to some embodiments, the codon optimized sequence is at least 85%, at least 90%, at least 95%, at least 96%, at least 97%, at least 98% or at least 99% identical to SEQ ID NO: 28.

According to some embodiments, the codon optimized sequence comprises SEQ ID NO: 29, shown below.

SEQ ID NO: 29
ATGAGCACCCTGTGCCCCCCCCCCAGCCCTGCCGTGGCCAAGACCGAGAT
CGCCCTCTCCGGCAAGTCCCCTCTGCTGGCCGCTACATTTGCCTACTGGG
ACAACATCCTCGGCCCTAGAGTGCGGCACATTTGGGCCCCTAAGACCGAA
CAGGTCCTCCTGAGCGACGGCGAAATAACATTTCTGGCCAACCACACCCT
GAACGGCGAAATCCTGAGAAACGCCGAGAGCGGCGCCATCGACGTGAAGT
TCTTCGTGCTGTCCGAGAAAGGCGTGATCATCGTGTCCCTGATCTTCGAC
GGCAACTGGAACGGAGATAGAAGCACATACGGACTGAGCATCATCCTCCC
ACAGACCGAGCTGTCTTTCTACCTGCCTCTGCACCGGGTGTGCGTGGACA
GACTGACCCACATCATTAGAAAGGGCAGAATCTGGATGCACAAGGAACGG
CAGGAGAACGTGCAAAAGATCATCCTGGAAGGGACCGAGCGTATGGAAGA
TCAGGGCCAGAGCATCATTCCTATGCTGACCGGCGAGGTGATCCCCGTGA
TGGAACTGCTGAGCAGCATGAAAAGCCACTCTGTGCCCGAGGAAATCGAC
ATCGCCGACACTGTGTTGAACGACGATGATATCGGCGATAGCTGCCACGA
GGGCTTCCTGCTGAACGCCATCAGCTCCCACCTGCAGACATGCGGCTGTA
GCGTTGTGGTGGGCTCTAGCGCCGAAAAAGTGAACAAGATCGTGCGGACC
CTTTGCCTGTTCCTGACACCTGCTGAGAGAAAGTGCAGCAGACTGTGTGA
AGCCGAATCTAGCTTTAAGTACGAGTCCGGACTCTTCGTGCAAGGCCTGC
TCAAGGACAGCACAGGCAGCTTCGTGCTGCCTTTCAGACAGGTGATGTAC
GCCCCTTACCCCACCACCCACATCGATGTCGACGTGAACACCGTGAAGCA
GATGCCTCCTTGCCACGAGCACATCTACAACCAGAGACGGTACATGAGAA
GCGAGCTGACCGCCTTTTGGCGGGCCACCAGCGAAGAGGACATGGCTCAA
GATACAATCATCTATACCGACGAGAGCTTTACCCCTGATCTGAACATCTT
TCAGGACGTGCTGCACAGAGATACCCTGGTGAAAGCCTTCCTGGATCAGG
TGTTCCAGCTGAAGCCTGGCCTGTCTCTGCGATCTACATTCCTCGCTCAG
TTCCTGCTGGTCCTGCATAGAAAGGCCCTGACTCTGATCAAGTACATCGA
GGACGACACACAGAAGGGCAAAAAGCCCTTCAAGTCTCTGCGGAACCTGA
AAATCGACCTGGACCTGACCGCCGAGGGCGACCTGAATATCATCATGGCC
CTGGCCGAGAAGATCAAACCCGGCCTGCACAGCTTCATCTTCGGAAGACC
TTTCTACACCAGCGTGCAGGAGAGAGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 29.

According to some embodiments, the codon optimized sequence comprises SEQ ID NO: 30, shown below.

SEQ ID NO: 30
ATGAGCACCCTGTGTCCTCCACCGAGCCCTGCCGTGGCCAAGACCGAGAT
AGCTCTGTCCGGCAAGTCCCCACTGCTGGCCGCCACCTTCGCCTACTGGG
ACAACATCCTGGGACCTAGAGTGCGGCACATCTGGGCCCCTAAGACGGAG
CAGGTCCTGCTGAGCGACGGCGAAATAACATTCCTGGCTAATCACACCCT
GAATGGCGAGATCCTGAGAAACGCCGAAAGCGGCGCCATCGACGTGAAGT
TCTTCGTGCTGTCTGAAAAGGGAGTGATCATCGTGTCCCTGATCTTCGAC
GGCAACTGGAACGGCGACCGGTCTACCTACGGCCTGAGCATCATCCTGCC
CCAGACCGAACTGTCTTTTTACCTGCCTCTGCACAGAGTGTGCGTGGACA
GACTGACCCACATCATCCGGAAGGGAAGAATCTGGATGCACAAGGAACGG
CAGGAGAACGTGCAAAAGATCATTCTCGAGGGCACCGAGAGAATGGAAGA
TCAGGGCCAGAGCATCATCCCCATGCTGACCGGCGAGGTGATCCCTGTGA
TGGAACTGCTGAGCAGCATGAAGTCCCACTCTGTGCCTGAGGAAATCGAC
ATCGCCGATACAGTGCTGAACGACGACGATATCGGCGACAGCTGCCACGA
GGGCTTCCTGCTGAACGCCATCAGCTCTCACCTGCAGACATGCGGCTGCA
GCGTGGTGGTGGGCAGCAGCGCCGAGAAGGTGAACAAGATCGTGCGGACC
CTTTGCCTGTTCTTGACCCCTGCTGAGAGAAAGTGCAGCAGACTGTGTGA
AGCCGAATCTAGCTTTAAGTACGAGTCTGGCCTCTTCGTGCAGGGACTGC
TGAAGGACAGCACAGGCAGCTTCGTGCTGCCTTTTAGACAGGTGATGTAC
GCCCCTTACCCTACAACACACATTGACGTGGACGTTAACACCGTGAAACA
GATGCCTCCATGTCACGAGCACATCTACAACCAGAGACGGTACATGCGGA
GCGAGCTGACAGCCTTTTGGCGGGCCACAAGCGAGGAAGATATGGCCCAA
GACACAATCATCTATACAGACGAGAGCTTCACCCCTGACCTGAACATCTT
TCAGGACGTGCTCCATAGAGATACCCTGGTGAAGGCCTTCCTGGACCAGG
TGTTCCAGCTGAAGCCCGGACTGAGCCTGAGATCTACATTCCTGGCCCAG
TTCCTGCTGGTGCTGCACAGAAAGGCCCTGACACTGATCAAGTACATCGA
GGATGATACACAGAAAGGCAAAAAGCCTTTCAAGAGCCTGCGGAACCTGA
AAATCGACCTGGATCTGACCGCCGAGGGAGATCTGAACATCATCATGGCC
CTGGCCGAAAAGATCAAGCCCGGCCTGCACAGCTTCATCTTCGGCAGACC
CTTCTACACCAGCGTGCAGGAGCGGGACGTTCTGATGACCTTTTGA

According to some embodiments, the codon optimized sequence is at least 85%, at least 90%, at least 95%, at least 96%, at least 97%, at least 98% or at least 99% identical to SEQ ID NO: 30.

According to some embodiments, the codon optimized sequence comprises SEQ ID NO: 31, shown below.

SEQ ID NO: 31
ATGAGCACCCTGTGCCCCCCCCCCAGCCCCGCCGTGGCCAAGACCGAGAT
CGCCCTGTCTGGAAAGAGCCCTCTGCTGGCCGCTACATTCGCCTACTGGG
ACAACATCCTGGGCCCTAGAGTGCGGCACATCTGGGCCCCTAAGACCGAA
CAGGTGCTGCTGAGTGATGGCGAGATCACCTTCCTGGCCAACCACACCCT
GAATGGAGAAATCCTGAGAAATGCCGAAAGCGGCGCCATCGACGTGAAGT
TCTTCGTGCTGAGCGAGAAGGGCGTGATCATCGTGTCCCTGATCTTCGAC
GGCAACTGGAACGGCGATAGAAGCACATACGGCCTGTCTATCATCCTGCC
TCAGACAGAGCTGAGCTTCTACCTGCCCCTGCACCGGGTGTGCGTGGACA
GACTGACACACATTATCCGGAAAGGCAGAATCTGGATGCACAAGGAACGG
CAGGAGAACGTGCAGAAAATCATCCTGGAAGGTACAGAACGGATGGAAGA
TCAGGGCCAGAGCATCATTCCTATGCTGACCGGCGAGGTGATCCCCGTGA
TGGAACTGCTATCCAGCATGAAAAGCCACTCTGTGCCTGAGGAAATCGAT
ATCGCCGACACCGTGCTGAACGACGACGACATCGGCGACTCTTGTCACGA
GGGCTTCCTGCTCAATGCTATCAGCAGCCACCTGCAGACCTGCGGCTGTT
CTGTGGTCGTGGGCAGCTCCGCCGAAAAGGTGAACAAGATAGTTAGAACC
CTGTGCCTGTTCCTGACCCCTGCCGAGCGGAAGTGCAGCAGACTGTGTGA
AGCCGAGTCCAGCTTTAAGTATGAGAGCGGACTGTTCGTTCAAGGCCTGC
TCAAGGACAGCACCGGCTCTTTTGTGCTCCCTTTTAGACAGGTCATGTAC
GCCCCTTACCCCACAACACACATCGACGTTGACGTGAACACCGTGAAGCA
GATGCCTCCTTGCCACGAGCACATCTACAACCAGAGACGGTACATGCGGA
GCGAGCTGACCGCCTTTTGGCGGGCCACATCTGAAGAGGACATGGCCCAG
GACACCATCATCTACACCGACGAGAGCTTCACACCTGACCTGAATATCTT
CCAAGACGTGCTGCACAGAGACACCCTGGTGAAAGCCTTCCTGGATCAGG
TGTTCCAGCTGAAACCTGGCCTGTCCCTGICGGAGCACCTTTCTGGCCCA
ATTTCTGCTCGTGCTTCATAGAAAGGCCCTGACGCTCATCAAGTACATCG
AGGATGACACACAGAAGGGCAAAAAGCCTTTCAAGTCCCTGAGAAACCTG
AAGATTGATCTGGACCTGACCGCCGAGGGAGATCTGAACATCATCATGGC
CCTGGCTGAGAAGATTAAGCCCGGCCTGCACAGCTTCATCTTCGGCAGAC
CTTTCTACACAAGCGTGCAGGAGCGGGACGTCCTCATGACCTTCTGA

According to some embodiments, the codon optimized sequence is at least 85%, at least 90%, at least 95%, at least 96%, at least 97%, at least 98% or at least 99% identical to SEQ ID NO: 31.

According to some embodiments, the codon optimized sequence comprises SEQ ID NO: 32, shown below.

SEQ ID NO: 32
ATGAGCACACTCTGCCCTCCTCCTAGCCCTGCCGTGGCCAAGACCGAGAT
CGCCCTGAGCGGAAAGTCTCCACTGCTGGCCGCTACATTCGCCTACTGGG
ACAACATACTGGGCCCTAGAGTGCGGCACATCTGGGCCCCTAAGACCGAG
CAGGTCCTCCTGAGTGATGGAGAAATCACCTTTCTGGCTAATCACACCCT
GAACGGCGAGATCCTGAGGAACGCCGAAAGCGGCGCCATCGACGTGAAGT
TCTTCGTTCTGAGCGAGAAGGGAGTGATCATCGTGTCCCTGATCTTCGAC
GGCAACTGGAACGGCGATAGATCTACATACGGCCTGAGCATCATCCTGCC
TCAGACAGAGCTGTCTTTCTACCTGCCTCTGCACAGAGTTTGTGTGGACC
GGCTGACCCACATCATCAGAAAAGGCCGGATCTGGATGCACAAGGAACGG
CAGGAGAACGTGCAGAAAATCATCCTGGAAGGCACCGAGCGGATGGAAGA
TCAGGGCCAGAGCATCATTCCTATGCTGACAGGCGAGGTGATCCCCGTGA
TGGAACTGCTGTCTTCTATGAAAAGCCACTCTGTGCCCGAGGAAATCGAC
ATCGCCGACACCGTGCTCAACGACGACGATATCGGCGACTCTTGTCACGA
AGGCTTCCTGCTGAATGCCATCAGCAGCCACCTGCAGACCTGCGGCTGTT
CTGTCGTGGTGGGCTCCAGCGCCGAAAAGGTGAACAAGATAGTTAGAACC
CTGTGCCTGTTCCTGACCCCTGCTGAAAGAAAGTGCAGCAGACTGTGCGA
GGCCGAGAGCAGCTTCAAGTACGAGAGCGGCCTGTTTGTGCAAGGCCTGC
TGAAGGACAGCACCGGCAGCTTCGTGCTGCCCTTCAGACAGGTGATGTAC
GCCCCTTATCCTACCACCCACATCGACGTGGACGTGAACACCGTGAAGCA
GATGCCCCCCTGCCACGAGCACATCTACAACCAGAGAAGATACATGAGAA
GCGAGCTGACCGCCTTCTGGCGGGCCACCAGCGAGGAAGATATGGCCCAA
GATACAATCATCTACACCGACGAGAGCTTTACACCTGATCTGAACATCTT
TCAGGACGTGCTGCACCGGGACACCCTGGTCAAGGCCTTTCTGGATCAGG
TGTTCCAGCTGAAGCCTGGACTGAGCCTGAGGTCCACCTTCCTGGCCCAG
TTCCTGCTGGTGCTGCATAGAAAGGCCCTGACCCTGATCAAGTACATCGA
GGACGACACACAGAAGGGCAAGAAGCCCTTTAAGTCCCTGCGGAACCTGA
AAATCGACCTGGACCTGACAGCCGAGGGCGACCTGAACATCATCATGGCT
CTGGCTGAGAAGATCAAACCCGGCCTGCACAGCTTCATCTTCGGCAGACC
TTTTTACACAAGCGTGCAAGAGAGAGATGTGCTGATGACCTTCTGA

According to some embodiments, the codon optimized sequence is at least 85%, at least 90%, at least 95%, at least 96%, at least 97%, at least 98% or at least 99% identical to SEQ ID NO: 32.

According to some embodiments, the codon optimized sequence comprises SEQ ID NO: 33, shown below.

SEQ ID NO: 33
ATGAGCACACTGTGTCCTCCTCCGAGCCCTGCCGTGGCCAAGACCGAGAT
CGCCCTGAGCGGCAAGTCCCCACTGCTTGCTGCTACCTTCGCCTACTGGG
ACAACATCCTGGGCCCTAGAGTGCGGCACATCTGGGCCCCTAAGACAGAG
CAGGTGCTGCTGAGCGACGGCGAAATAACATTCCTGGCCAACCACACCCT
GAACGGCGAGATCCTGAGAAACGCCGAGAGCGGCGCTATCGACGTGAAGT
TCTTCGTTCTGTCTGAAAAGGGCGTGATCATCGTGTCCCTGATCTTCGAC
GGCAACTGGAACGGCGATAGAAGCACCTACGGCCTGAGCATTATCCTGCC
TCAGACAGAACTGTCTTTCTACCTGCCTCTGCACAGAGTGTGCGTGGACA
GACTGACACACATCATTAGAAAGGGCAGAATCTGGATGCACAAGGAAAGA
CAGGAGAACGTGCAGAAGATCATCCTGGAAGGCACCGAGAGAATGGAAGA
TCAGGGCCAGTCTATCATCCCTATGCTGACCGGCGAGGTGATCCCCGTGA
TGGAACTGCTGTCTAGCATGAAAAGCCACTCTGTGCCCGAGGAAATCGAC
ATCGCCGATACAGTGCTGAACGACGATGATATAGGAGATAGCTGCCATGA
GGGCTTCCTGCTGAACGCCATCAGCTCCCACCTGCAGACCTGCGGATGTA
GCGTGGTCGTGGGCTCCTCCGCCGAGAAGGTGAACAAGATCGTGCGGACC
CTGTGCCTGTTCCTGACACCTGCTGAACGGAAGTGCAGCAGACTGTGCGA
GGCCGAATCTTCTTTTAAGTACGAGAGCGGACTGTTCGTGCAAGGCCTGC
TGAAGGACAGCACCGGCAGCTTTGTGCTGCCATTCCGGCAGGTGATGTAC
GCCCCTTACCCCACCACCCACATTGACGTCGACGTGAACACCGTGAAGCA
GATGCCCCCCTGTCACGAGCACATCTACAACCAGAGGCGGTACATGAGAA
GCGAGCTGACAGCCTTTTGGCGGGCCACCAGCGAGGAAGATATGGCCCAA
GACACCATCATCTACACCGACGAGAGCTTCACCCCTGATCTGAATATCTT
TCAGGACGTGCTGCACAGAGATACACTGGTGAAAGCCTTCCTGGACCAGG
TTTTCCAGCTGAAGCCTGGCCTGAGCCTGCGCAGCACCTTTCTGGCCCAG
TTCCTGCTCGTGCTGCACCGGAAGGCCCTGACACTGATTAAGTACATCGA
GGACGACACCCAGAAAGGAAAAAAGCCCTTCAAGAGCCTGCGGAACCTGA
AAATCGACCTGGACCTGACCGCCGAGGGCGACCTGAACATCATCATGGCC
CTGGCCGAAAAGATCAAACCTGGACTGCATTCTTTCATCTTCGGCAGACC
TTTTTACACCAGCGTGCAGGAGCGGGACGTTCTGATGACCTTCTGA

According to some embodiments, the codon optimized sequence is at least 85%, at least 90%, at least 95%, at least 96%, at least 97%, at least 98% or at least 99% identical to SEQ ID NO: 33.

According to some embodiments, the codon optimized sequence comprises SEQ ID NO: 34, shown below.

SEQ ID NO: 34
ATGTCTACACTCTGTCCTCCACCTAGCCCTGCTGTGGCCAAGACAGAAAT
CGCCCTGAGCGGAAAAAGCCCCCTGCTGGCCGCCACCTTCGCCTACTGGG
ACAACATCCTGGGCCCCAGAGTCAGACACATCTGGGCCCCTAAGACCGAG
CAGGTGCTGCTGAGCGACGGAGAGATCACCTTCCTGGCCAACCACACCCT
GAATGGCGAGATCCTGCGGAACGCCGAGTCTGGCGCCATCGACGTGAAGT
TCTTCGTGCTGTCTGAGAAAGGCGTGATCATTGTGTCCCTCATCTTTGAC
GGCAACTGGAACGGAGATAGAAGCACCTACGGCCTGTCCATCATCCTGCC
CCAGACAGAGCTGAGCTTCTACCTGCCTCTGCACAGAGTGTGCGTGGACA
GACTGACCCACATCATCAGAAAGGGCAGAATCTGGATGCACAAGGAACGG
CAGGAGAACGTGCAAAAAATCATCCTGGAAGGCACCGAGAGAATGGAAGA
TCAGGGCCAGAGCATCATCCCCATGCTGACCGGCGAGGTGATCCCTGTGA
TGGAACTGCTGAGCAGCATGAAGTCCCATTCTGTCCCCGAGGAAATCGAC
ATCGCCGACACCGTGCTGAACGACGATGATATCGGCGATAGCTGCCACGA
GGGCTTCCTGCTGAACGCCATCAGCTCTCACCTGCAGACCTGCGGCTGCA
GCGTGGTGGTCGGCTCTTCCGCCGAAAAGGTGAACAAGATCGTGCGGACC
CTGTGCCTGTTCCTGACTCCTGCCGAAAGAAAGTGCTCTAGACTGTGTGA
AGCCGAGAGCAGCTTCAAATACGAGTCCGGTCTTTTTGTGCAGGGGCTGC
TGAAGGACAGCACAGGCAGCTTCGTGCTTCCATTCAGACAGGTGATGTAC
GCCCCTTACCCCACAACACACATTGATGTGGACGTGAACACCGTGAAGCA
GATGCCTCCTTGCCACGAGCACATCTACAACCAGCGGAGATACATGCGGA
GCGAGCTGACAGCCTTCTGGCGGGCCACAAGCGAGGAAGATATGGCCCAG
GACACCATCATCTACACCGACGAGAGCTTCACCCCTGATCTGAATATCTT
CCAAGACGTCCTGCACCGCGACACACTCGTGAAAGCCTTTCTCGACCAGG
TTTTCCAGCTGAAACCTGGCCTGAGTCTGAGATCCACCTTCCTGGCTCAA
TTTCTGCTGGTGCTCCACCGGAAGGCCCTGACCCTGATCAAGTACATCGA
GGACGACACCCAGAAGGGCAAGAAGCCTTTCAAGTCTCTGAGAAACCTGA
AGATCGACCTGGACCTGACAGCTGAGGGCGACCTGAATATCATCATGGCC
CTTGCTGAGAAGATCAAGCCCGGCCTGCACAGCTTCATCTTCGGCAGACC
TTTTTATACCAGCGTGCAGGAGAGAGATGTGCTGATGACCTTCTGA

According to some embodiments, the codon optimized sequence is at least 85%, at least 90%, at least 95%, at least 96%, at least 97%, at least 98% or at least 99% identical to SEQ ID NO: 34.

According to some embodiments, the codon optimized sequence comprises SEQ ID NO: 35, shown below.

SEQ ID NO: 35
ATGAGCACCCTGTGTCCTCCACCTAGCCCCGCCGTGGCCAAGACCGAGAT
CGCCCTGTCTGGAAAGTCCCCTCTGCTGGCCGCTACATTCGCCTACTGGG
ACAACATCCTGGGACCTAGAGTGCGGCACATCTGGGCCCCTAAGACCGAG
CAGGTGCTCCTGAGTGATGGCGAGATAACATTTCTGGCCAACCACACCCT
CAACGGCGAGATCCTGAGAAACGCCGAAAGCGGCGCCATCGACGTGAAGT
TCTTCGTGCTGTCTGAAAAGGGCGTGATCATCGTGTCCCTGATCTTCGAC
GGCAACTGGAACGGCGACAGAAGCACGTACGGCCTGTCCATCATCCTGCC
CCAGACCGAGCTGTCTTTCTACCTGCCTCTGCACCGGGTGTGCGTGGATA
GACTGACCCACATTATTAGAAAGGGCAGAATCTGGATGCACAAGGAACGC
CAGGAGAACGTGCAGAAGATCATCCTGGAAGGTACAGAGCGGATGGAAGA
TCAGGGCCAGAGCATCATCCCCATGCTGACCGGCGAAGTGATCCCTGTGA
TGGAACTGCTGAGTTCTATGAAAAGCCACAGCGTGCCGGAAGAGATCGAT
ATCGCCGACACCGTCCTTAACGACGACGACATAGGAGATAGCTGCCACGA
GGGCTTCCTTCTGAACGCCATCAGCTCTCACCTGCAGACATGCGGCTGCA
GCGTCGTGGTCGGCTCTAGCGCCGAAAAAGTGAACAAGATCGTGCGGACC
CTGTGCCTGTTCCTGACACCTGCCGAGAGAAAGTGCTCTAGACTGTGCGA
GGCCGAGTCCAGCTTCAAGTACGAGAGCGGCCTGTTTGTTCAAGGACTGC
TGAAGGACAGCACCGGCAGCTTTGTGCTCCCTTTTAGACAGGTGATGTAC
GCCCCTTACCCCACCACCCACATCGACGTTGACGTGAATACCGTGAAACA
GATGCCTCCTTGTCACGAGCACATCTACAACCAGAGAAGATACATGAGAT
CTGAGCTGACCGCCTTCTGGCGGGCCACCAGCGAGGAAGATATGGCCCAG
GACACCATCATCTACACCGACGAGAGCTTCACCCCTGATCTGAACATCTT
TCAGGATGTCCTGCACCGCGACACCCTGGTCAAAGCCTTTCTGGACCAGG
TGTTCCAGCTGAAACCCGGACTGTCTCTGCGGAGCACCTTCTTGGCTCAA
TTTCTCCTGGTGCTGCACAGAAAGGCCCTGACACTGATCAAGTACATCGA
GGATGATACACAGAAAGGCAAAAAGCCCTTCAAGAGCCTGAGAAATCTGA
AGATCGACCTGGACCTGACAGCCGAGGGCGATCTGAACATCATCATGGCC
CTGGCTGAGAAGATTAAGCCTGGCCTCCATTCTTTCATCTTCGGCAGACC
TTTCTACACCAGCGTGCAGGAGCGGGACGTGCTGATGACATTCTGA

According to some embodiments, the codon optimized sequence is at least 85%, at least 90%, at least 95%, at least 96%, at least 97%, at least 98% or at least 99% identical to SEQ ID NO: 35.

According to some embodiments, the codon optimized sequence comprises SEQ ID NO: 36, shown below.

SEQ ID NO: 36
ATGAGCACCCTGTGTCCTCCTCCATCTCCAGCCGTGGCCAAGACCGAGAT
CGCCCTGTCCGGCAAGAGCCCTCTGCTGGCCGCTACATTCGCCTACTGGG
ACAACATCCTGGGACCTAGAGTGCGGCACATCTGGGCCCCTAAGACAGAG
CAGGTGCTGCTGAGTGATGGCGAGATCACCTTCCTGGCCAACCACACCCT
GAATGGAGAAATCCTGAGAAACGCCGAGAGTGGCGCCATCGATGTGAAGT
TCTTCGTGCTGTCTGAAAAGGGCGTGATCATCGTCAGCCTGATCTTCGAC
GGCAACTGGAACGGCGACAGAAGCACATACGGCCTGAGCATCATCCTGCC
CCAGACAGAGCTGTCTTTTTACCTGCCTCTGCACAGAGTGTGCGTGGACC
GGCTGACCCACATCATTAGAAAGGGCAGAATCTGGATGCACAAGGAAAGA
CAGGAGAACGTGCAGAAGATCATCCTGGAAGGTACAGAGAGAATGGAAGA
TCAGGGACAGAGCATCATCCCCATGCTGACCGGCGAAGTGATCCCTGTGA
TGGAACTGCTGAGCAGCATGAAAAGCCATTCTGTGCCCGAGGAAATCGAC
ATCGCCGACACAGTGCTGAACGACGACGATATCGGCGATAGCTGCCACGA
GGGATTCCTGCTTAATGCCATCAGCAGCCACCTGCAGACCTGTGGCTGTA
GCGTGGTCGTGGGCAGCTCCGCCGAGAAGGTGAACAAGATCGTGAGGACC
CTCTGCCTGTTCCTGACACCTGCTGAAAGAAAGTGCAGCAGACTGTGCGA
GGCCGAGTCCAGCTTCAAGTACGAGAGCGGCCTCTTCGTGCAGGGCCTGC
TGAAGGACAGCACCGGCTCCTTCGTGCTGCCTTTTAGACAGGTGATGTAC
GCCCCTTACCCCACCACCCACATTGACGTGGACGTGAACACCGTGAAGCA
GATGCCTCCGTGCCACGAGCACATCTACAACCAGCGCAGATACATGCGGA
GCGAGCTGACCGCCTTCTGGCGGGCCACATCTGAGGAAGATATGGCTCAA
GATACCATCATCTACACCGACGAGAGCTTCACCCCTGATCTGAACATCTT
CCAGGACGTGCTGCATAGAGATACCCTGGTGAAAGCTTTCCTTGATCAGG
TTTTCCAACTGAAGCCTGGCCTGAGCCTGAGAAGCACCTTCCTGGCTCAG
TTCCTGCTGGTGCTTCACCGGAAGGCCCTAACCCTGATCAAGTACATCGA
GGATGACACCCAGAAAGGCAAAAAGCCTTTTAAGTCCCTGCGGAACCTGA
AAATCGACCTGGACCTCACAGCCGAGGGAGATCTGAACATCATCATGGCC
CTGGCCGAAAAGATAAAGCCCGGCCTGCACAGCTTCATCTTTGGCAGACC
TTTCTACACAAGCGTGCAGGAGCGGGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 36.

According to some embodiments, the codon optimized sequence comprises SEQ ID NO: 37, shown below.

SEQ ID NO: 37
ATGAGCACCCTCTGTCCTCCACCTAGCCCTGCTGTGGCCAAGACCGAAAT
TGCCCTGAGCGGAAAGTCTCCTCTGTTGGCTGCTACATTCGCCTACTGGG
ACAACATCCTGGGCCCTAGAGTGCGGCACATCTGGGCCCCTAAGACAGAG
CAGGTGCTGCTGAGTGATGGCGAAATCACCTTCCTGGCCAACCACACCCT
GAACGGCGAGATCCTGAGAAACGCCGAAAGCGGCGCCATCGACGTGAAGT
TCTTCGTGCTGTCTGAAAAGGGTGTTATCATTGTGTCCCTGATCTTTGAC
GGCAACTGGAACGGCGACAGATCTACATACGGCCTGTCCATCATCCTGCC
TCAGACCGAGCTGTCTTTCTACCTGCCTCTGCACAGAGTGTGCGTGGACC
GGCTGACTCATATCATCAGAAAGGGAAGAATCTGGATGCACAAGGAAAGA
CAGGAGAACGTGCAGAAGATCATCCTGGAAGGTACAGAGAGAATGGAAGA
TCAGGGCCAGAGCATCATCCCCATGCTGACAGGCGAGGTGATCCCTGTGA
TGGAACTGCTGAGCAGCATGAAGTCCCACAGCGTCCCCGAGGAAATCGAC
ATCGCCGACACAGTGCTGAACGACGACGATATCGGCGATTCATGCCACGA
GGGCTTCCTGCTGAATGCAATCAGCAGCCACCTGCAGACCTGCGGCTGTT
CTGTGGTGGTGGGCAGCAGCGCCGAAAAAGTGAACAAGATCGTGCGCACC
CTGTGCCTGTTTTTGACCCCTGCCGAGCGGAAGTGCAGCAGACTGTGTGA
AGCCGAGAGCTCTTTCAAGTACGAGAGCGGCCTGTTCGTTCAAGGCCTGC
TGAAGGACAGCACCGGCAGCTTTGTGCTGCCCTTCCGGCAGGTGATGTAC
GCCCCTTACCCCACCACCCACATCGACGTCGACGTGAACACCGTGAAGCA
GATGCCTCCGTGCCACGAGCACATCTACAACCAGCGGAGATACATGCGGT
CCGAGCTGACAGCCTTCTGGCGGGCCACCAGCGAAGAGGACATGGCCCAG
GACACCATCATCTACACTGATGAGTCCTTCACACCTGATCTGAATATCTT
CCAAGACGTGCTTCACAGAGACACCCTGGTGAAAGCTTTTCTCGACCAGG
TTTTCCAGCTGAAGCCCGGCCTGAGCCTGAGATCTACCTTCCTGGCTCAA
TTTCTGCTCGTGCTGCACAGAAAGGCCCTGACGCTGATCAAGTATATCGA
GGACGACACGCAGAAAGGCAAGAAACCCTTCAAAAGCCTGCGGAACCTGA
AAATTGACCTGGACCTGACCGCCGAGGGCGACCTGAACATCATCATGGCC
CTGGCCGAGAAGATCAAGCCTGGACTGCATAGCTTCATCTTCGGCAGACC
TTTTTACACCTCTGTGCAGGAGCGGGACGTGCTCATGACCTTTTGA

According to some embodiments, the codon optimized sequence is at least 85%, at least 90%, at least 95%, at least 96%, at least 97%, at least 98% or at least 99% identical to SEQ ID NO: 37.

According to some embodiments, the codon optimized sequence comprises SEQ ID NO: 38, shown below.

SEQ ID NO: 38
ATGAGCACCCTGTGTCCTCCTCCAAGCCCTGCCGTGGCCAAGACAGAGAT
CGCCCTTAGCGGAAAGTCCCCTCTGCTGGCCGCCACATTTGCCTACTGGG
ACAACATCCTGGGACCTAGAGTGCGGCACATTTGGGCCCCAAAGACCGAG
CAGGTGCTGCTGAGCGACGGCGAAATCACCTTCCTGGCTAATCACACACT
GAACGGCGAGATCCTGAGGAACGCCGAAAGCGGCGCCATCGACGTGAAGT
TCTTCGTCCTGAGCGAGAAGGGCGTGATCATTGTGTCCCTGATCTTCGAC
GGCAACTGGAACGGCGACCGCTCCACATACGGCCTGTCTATCATCCTGCC
CCAGACCGAGCTGTCTTTTTACCTGCCTCTGCACAGAGTGTGCGTGGACA
GACTGACCCACATCATCCGGAAGGGCAGAATCTGGATGCACAAGGAACGG
CAGGAGAACGTGCAGAAAATCATCCTGGAAGGAACAGAGCGGATGGAAGA
TCAGGGCCAGAGCATCATACCCATGCTGACTGGCGAGGTGATCCCTGTGA
TGGAACTGCTGTCAAGCATGAAAAGCCACTCTGTCCCCGAGGAAATCGAC
ATCGCTGATACCGTGCTCAACGACGACGATATCGGCGATAGCTGCCACGA
GGGCTTCCTGCTGAACGCCATCAGCAGCCACCTGCAGACATGCGGCTGCA
GCGTCGTGGTGGGCTCTAGCGCCGAAAAGGTGAACAAGATCGTGCGGACC
CTGTGTCTGTTCTTGACCCCTGCTGAAAGAAAGTGCAGCAGACTGTGCGA
GGCCGAGAGCAGCTTCAAGTACGAGTCTGGCCTGTTTGTGCAGGGCCTGC
TGAAAGACAGCACAGGCAGCTTCGTGCTGCCCTTCAGACAGGTGATGTAC
GCCCCTTACCCTACCACCCACATTGACGTGGACGTGAACACCGTGAAGCA
GATGCCTCCGTGCCACGAGCACATCTACAACCAGCGTAGATACATGAGAT
CCGAGCTGACAGCTTTCTGGCGGGCCACCTCTGAAGAGGATATGGCCCAG
GACACCATCATCTATACCGACGAGAGCTTCACCCCTGATCTGAATATCTT
CCAAGACGTGCTGCATAGAGACACCCTGGTGAAAGCCTTCCTGGATCAAG
TGTTCCAGCTGAAGCCTGGACTGAGCCTGCGGAGCACCTTCCTGGCCCAG
TTCCTGCTCGTGCTTCATAGAAAGGCCCTGACACTGATCAAGTACATCGA
GGACGACACACAGAAGGGCAAAAAGCCCTTCAAGAGCCTGAGAAACCTGA
AGATCGACCTGGACCTGACCGCCGAGGGCGATCTGAACATCATCATGGCT
CTGGCCGAGAAGATCAAGCCCGGCCTGCACAGCTTTATCTTTGGCAGACC
TTTCTACACCAGCGTGCAAGAGAGAGATGTGCTGATGACCTTTTGA

According to some embodiments, the codon optimized sequence is at least 85%, at least 90%, at least 95%, at least 96%, at least 97%, at least 98% or at least 99% identical to SEQ ID NO: 38.

According to some embodiments, the codon optimized sequence comprises SEQ ID NO: 39, shown below.

SEQ ID NO: 39
ATGTCTACCCTGTGTCCTCCTCCAAGCCCCGCCGTGGCCAAGACTGAGAT
CGCCCTGAGCGGCAAATCTCCTCTGCTCGCTGCTACCTTCGCCTACTGGG
ACAACATCCTGGGACCTAGAGTGCGGCACATCTGGGCCCCTAAGACCGAG
CAGGTCCTGCTGAGCGACGGAGAGATAACATTTCTGGCCAACCACACACT
GAACGGCGAGATCCTCAGAAATGCCGAGAGCGGCGCCATCGACGTGAAGT
TCTTCGTGCTGTCTGAGAAGGGCGTGATCATTGTGTCCCTGATCTTCGAC
GGCAACTGGAACGGCGACAGAAGCACCTACGGCCTGAGCATCATCCTGCC
TCAGACAGAGCTGTCCTTTTACCTGCCACTGCACCGGGTGTGCGTGGATA
GACTGACACACATCATTAGAAAGGGCAGAATCTGGATGCACAAGGAAAGA
CAGGAGAACGTGCAGAAAATCATCCTGGAAGGTACAGAGCGGATGGAAGA
TCAGGGCCAGAGCATCATCCCTATGCTGACCGGCGAGGTGATCCCCGTTA
TGGAACTCCTGTCTTCTATGAAAAGCCACAGCGTCCCCGAGGAAATCGAC
ATCGCAGATACAGTGCTGAACGACGACGATATAGGAGATAGCTGTCACGA
GGGCTTCCTGTTAAACGCCATCAGCAGCCACCTGCAGACCTGTGGCTGCA
GCGTGGTGGTCGGCTCTAGCGCCGAAAAGGTGAACAAGATCGTGCGGACC
CTGTGCCTGTTCCTGACACCTGCTGAACGGAAGTGCAGCAGACTGTGCGA
GGCCGAGAGCAGTTTTAAGTACGAGTCCGGCCTGTTCGTGCAAGGCCTGC
TGAAGGACTCTACAGGCAGCTTCGTGCTGCCTTTCAGACAGGTGATGTAC
GCCCCTTACCCCACCACCCACATCGACGTGGACGTGAACACCGTGAAGCA
GATGCCTCCGTGCCACGAGCACATCTACAACCAGCGGAGATACATGCGGA
GCGAGCTGACCGCTTTCTGGCGGGCCACCAGCGAAGAGGACATGGCTCAG
GACACCATCATCTATACAGACGAGAGCTTCACCCCTGACCTGAATATCTT
TCAAGACGTGCTGCACAGAGATACCCTCGTGAAAGCCTTCCTGGACCAGG
TGTTCCAGCTGAAACCTGGACTGTCACTGAGAAGCACCTTTCTGGCCCAG
TTCCTGCTGGTCCTGCACAGAAAGGCCCTGACCCTTATCAAGTACATCGA
GGATGACACCCAGAAGGGCAAGAAGCCCTTCAAGAGCCTGAGAAACCTGA
AGATCGACCTGGATCTGACAGCCGAAGGCGACCTGAACATCATCATGGCC
CTGGCCGAAAAGATTAAGCCTGGCCTGCATTCTTTCATCTTCGGCCGCCC
CTTCTACACCAGCGTGCAGGAGAGAGATGTGCTGATGACCTTCTGA

According to some embodiments, the codon optimized sequence is at least 85%, at least 90%, at least 95%, at least 96%, at least 97%, at least 98% or at least 99% identical to SEQ ID NO: 39.

According to some embodiments, the codon optimized sequence comprises SEQ ID NO: 40, shown below.

SEQ ID NO: 40
ATGAGCACCCTGTGTCCTCCTCCTAGCCCTGCCGTGGCAAAGACCGAGAT
CGCCCTGAGCGGGAAGTCACCCCTGCTGGCCGCTACATTTGCCTACTGGG
ACAACATCCTGGGCCCTAGAGTGCGGCACATCTGGGCCCCTAAGACCGAG
CAGGTGCTGCTCAGTGATGGCGAGATAACATTCCTCGCCAACCACACACT
GAATGGCGAAATCCTTAGAAATGCCGAGAGCGGTGCTATCGACGTAAAGT
TCTTCGTGCTGTCTGAAAAGGGCGTGATCATCGTGTCCCTGATCTTCGAC
GGCAACTGGAACGGCGATAGAAGCACCTACGGCCTGAGCATCATCCTGCC
TCAGACAGAGCTGAGCTTCTATCTGCCTCTGCACAGGGTGTGCGTGGACA
GACTGACTCACATTATTAGAAAAGGCAGAATCTGGATGCACAAGGAAAGA
CAGGAGAACGTGCAAAAGATCATCCTGGAAGGCACCGAGAGAATGGAAGA
TCAGGGCCAGAGCATCATCCCTATGCTGACCGGCGAGGTGATCCCCGTGA
TGGAACTGCTGAGTTCTATGAAGAGTCACTCTGTGCCCGAGGAAATCGAC
ATCGCCGACACAGTGCTGAACGACGACGATATCGGCGACTCCTGCCACGA
GGGCTTCCTGCTGAACGCCATCAGCAGCCACCTGCAGACCTGCGGCTGCA
GCGTGGTGGTCGGCAGCTCCGCCGAAAAGGTGAACAAGATCGTGCGGACC
CTGTGCCTGTTCCTGACGCCCGCCGAAAGAAAGTGCAGTAGACTGTGCGA
GGCCGAAAGCTCTTTCAAGTACGAGAGCGGCCTGTTTGTGCAGGGCCTGC
TCAAGGACAGCACTGGATCTTTCGTGCTCCCCTTCAGACAGGTGATGTAC
GCCCCTTACCCTACAACACACATCGATGTGGACGTGAACACCGTGAAGCA
GATGCCTCCATGTCACGAGCACATCTACAACCAGCGTAGATACATGAGAA
GCGAGCTGACAGCCTTTTGGCGGGCCACAAGCGAGGAAGATATGGCCCAG
GACACCATCATCTACACCGACGAGAGCTTCACCCCTGACCTGAATATCTT
TCAGGACGTTCTGCACCGGGACACCCTTGTGAAGGCCTTCCTGGACCAGG
TTTTCCAGCTGAAACCTGGCCTCTCCCTGCGGAGCACATTCCTGGCTCAG
TTCCTGCTGGTGCTGCATAGAAAGGCCCTGACACTGATCAAGTACATCGA
GGATGACACCCAGAAGGGCAAAAAGCCTTTTAAGAGCCTGAGAAACCTGA
AGATCGACCTGGATCTGACCGCCGAGGGCGACCTGAACATCATCATGGCT
CTGGCCGAGAAAATCAAGCCCGGACTGCATAGCTTCATCTTCGGAAGACC
TTTCTACACCAGCGTGCAGGAGCGGGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 40.

According to some embodiments, the codon optimized sequence comprises SEQ ID NO: 41, shown below.

SEQ ID NO: 41
ATGAGCACACTGTGCCCCCCCCCGAGCCCGGCCGTGGCCAAGACAGAGAT
CGCCCTGAGCGGCAAGTCCCCTCTGCTGGCCGCCACCTTCGCCTACTGGG
ACAACATCCTGGGCCCTAGAGTGCGGCACATCTGGGCCCCTAAGACCGAG
CAGGTTCTGCTGAGTGATGGCGAGATAACATTCCTGGCCAACCACACCCT
GAACGGCGAGATCCTGAGAAATGCCGAATCTGGCGCCATCGACGTGAAGT
TCTTCGTGCTGTCTGAGAAGGGCGTGATCATTGTGTCCCTGATCTTCGAC
GGCAACTGGAACGGCGATAGAAGCACCTACGGCCTGAGCATCATCCTGCC
ACAGACCGAACTGTCGTTCTACCTGCCTCTGCACCGAGTGTGCGTGGACA
GACTGACCCACATCATCAGAAAGGGAAGAATCTGGATGCACAAGGAAAGA
CAGGAGAACGTGCAGAAGATCATCCTGGAAGGTACAGAACGGATGGAAGA
TCAGGGACAGAGCATCATCCCCATGCTGACAGGCGAAGTGATCCCTGTGA
TGGAACTGCTGAGCTCTATGAAAAGCCACAGCGTGCCTGAGGAAATCGAC
ATCGCTGATACCGTGCTGAACGACGACGATATCGGCGACAGCTGCCACGA
GGGCTTCCTGCTGAACGCCATCAGCAGTCACCTGCAGACATGCGGCTGTA
GCGTCGTGGTGGGCTCCAGCGCCGAGAAAGTGAACAAGATCGTGCGCACC
CTGTGCCTGTTCCTGACCCCTGCTGAGCGGAAATGCAGCAGACTGTGTGA
AGCCGAGAGCTCCTTTAAGTACGAGAGCGGCCTTTTTGTGCAGGGCCTGC
TGAAGGACAGCACAGGCAGCTTCGTGCTGCCCTTCCGGCAGGTGATGTAC
GCCCCTTATCCTACCACCCACATCGACGTCGACGTGAACACCGTGAAGCA
GATGCCTCCTTGCCACGAGCACATCTACAACCAGAGAAGATACATGAGAT
CCGAGCTGACCGCCTTCTGGCGGGCCACAAGCGAGGAAGATATGGCCCAA
GACACCATCATCTACACTGATGAGAGTTTCACCCCTGATCTGAACATCTT
TCAGGACGTGCTCCATCGGGACACCCTGGTGAAAGCTTTCCTGGATCAAG
TCTTTCAGCTGAAGCCCGGCCTGTCCCTGCGGTCCACCTTCCTGGCCCAG
TTCCTGCTCGTGCTGCACCGGAAGGCCCTGACCCTGATCAAATACATCGA
GGACGACACACAGAAAGGCAAAAAGCCTTTCAAGAGCCTGAGAAACCTGA
AAATCGATCTGGACCTGACAGCCGAGGGCGACCTGAATATCATCATGGCC
CTGGCTGAAAAGATTAAGCCCGGACTGCATTCTTTCATCTTCGGCAGACC
TTTCTACACCAGCGTGCAGGAGAGAGATGTCCTCATGACCTTTTGA

According to some embodiments, the codon optimized sequence is at least 85%, at least 90%, at least 95%, at least 96%, at least 97%, at least 98% or at least 99% identical to SEQ ID NO: 41.

According to some embodiments, the codon optimized sequence comprises SEQ ID NO: 42, shown below.

SEQ ID NO: 42
ATGAGCACATTGTGTCCTCCACCATCTCCTGCCGTGGCCAAGACCGAAAT
CGCCCTGAGCGGCAAGAGCCCCCTGCTCGCCGCCACCTTCGCCTACTGGG
ACAACATCCTGGGCCCTAGAGTGCGGCACATCTGGGCCCCTAAGACCGAG
CAGGTTCTGCTGAGCGACGGCGAGATAACATTCCTGGCTAATCACACCCT
GAATGGCGAGATCCTGCGGAACGCCGAAAGCGGAGCCATCGACGTGAAGT
TCTTCGTGCTGAGCGAGAAGGGAGTGATCATCGTGTCCCTGATCTTCGAC
GGCAACTGGAACGGCGACCGCTCCACCTACGGCCTGTCTATCATCCTGCC
TCAGACCGAGCTGAGTTTCTACCTGCCTCTGCACCGGGTGTGCGTGGACA
GACTGACACACATCATCCGGAAAGGCAGAATCTGGATGCACAAGGAACGG
CAGGAGAACGTGCAAAAGATCATCCTGGAAGGCACCGAGAGAATGGAAGA
TCAGGGCCAGAGCATCATTCCCATGCTGACTGGAGAAGTGATCCCTGTGA
TGGAACTGCTGAGCAGCATGAAGTCCCACAGCGTGCCCGAGGAAATCGAC
ATCGCCGACACCGTGCTGAACGACGATGACATAGGAGATTCATGCCACGA
GGGCTTCCTGCTGAACGCCATCAGCTCTCACCTGCAGACATGCGGCTGTA
GCGTCGTGGTGGGCTCTAGCGCCGAAAAGGTGAACAAGATCGTCAGAACC
CTGTGCCTGTTCCTGACCCCTGCTGAAAGAAAGTGCAGCCGGCTGTGCGA
GGCCGAGTCCAGTTTTAAGTACGAGAGCGGCTTGTTTGTGCAGGGACTGC
TGAAGGACAGCACCGGCAGCTTCGTGCTCCCCTTCAGACAGGTGATGTAC
GCCCCTTATCCTACAACCCACATTGATGTGGATGTTAACACCGTGAAGCA
GATGCCTCCATGTCATGAGCACATCTACAACCAGCGTAGATACATGCGGA
GCGAGCTGACCGCCTTTTGGCGGGCCACAAGCGAGGAAGATATGGCCCAG
GATACCATCATCTACACAGACGAGAGCTTCACCCCTGATCTGAATATCTT
CCAAGACGTCCTGCACAGAGACACCCTCGTGAAGGCCTTCCTGGACCAGG
TGTTCCAGCTGAAACCCGGCCTGAGCCTGAGAAGCACCTTCCTCGCTCAG
TTCCTGCTGGTGCTGCATAGAAAGGCCCTGACCCTGATCAAGTACATCGA
GGACGACACACAGAAAGGAAAAAAGCCCTTCAAGAGCCTGAGAAACCTGA
AGATCGACCTGGATCTGACAGCCGAGGGCGATCTGAACATCATCATGGCT
CTGGCCGAGAAGATCAAGCCTGGCCTCCACTCCTTCATCTTCGGCAGACC
TTTTTACACCAGCGTGCAAGAGCGGGACGTGCTCATGACCTTTTGA

According to some embodiments, the codon optimized sequence is at least 85%, at least 90%, at least 95%, at least 96%, at least 97%, at least 98% or at least 99% identical to SEQ ID NO: 42.

According to some embodiments, the codon optimized sequence comprises SEQ ID NO: 43, shown below.

SEQ ID NO: 43
ATGAGCACCCTGTGCCCCCCCCCCAGCCCAGCCGTGGCCAAGACCGAGAT
AGCTCTGAGCGGAAAAAGCCCTCTGCTGGCCGCCACCTTCGCCTACTGGG
ACAACATCCTGGGGCCTAGAGTCAGACACATCTGGGCCCCTAAGACCGAG
CAGGTGCTGCTGAGCGACGGAGAGATCACCTTCCTGGCTAATCACACCCT
GAATGGCGAGATCCTGAGAAACGCCGAAAGCGGCGCCATCGACGTGAAGT
TCTTCGTGCTGTCTGAAAAGGGCGTGATCATCGTCAGCCTGATCTTCGAC
GGCAACTGGAACGGCGACAGAAGCACATACGGCCTGTCTATCATTCTGCC
TCAGACAGAGCTGAGTTTTTACCTGCCTCTGCACCGGGTGTGCGTGGACC
GGCTGACCCACATCATTAGAAAGGGAAGAATCTGGATGCACAAGGAACGG
CAGGAGAACGTGCAGAAAATCATCCTGGAAGGGACCGAGAGAATGGAAGA
TCAGGGCCAGAGCATCATCCCCATGCTGACCGGCGAAGTGATCCCTGTGA
TGGAACTGCTGTCTTCTATGAAAAGCCACTCTGTGCCCGAGGAAATCGAT
ATCGCCGATACAGTGCTGAACGACGACGACATCGGCGACTCATGCCACGA
GGGCTTCCTTCTGAACGCCATCAGCTCTCACCTGCAGACCTGTGGCTGCA
GCGTGGTCGTGGGCAGCAGCGCCGAGAAAGTGAACAAGATCGTGCGGACC
CTGTGTCTGTTCCTCACACCTGCCGAGCGGAAGTGCAGTAGACTGTGCGA
GGCCGAATCCAGCTTTAAGTACGAGAGCGGCCTGTTCGTGCAGGGCCTGC
TGAAAGACAGCACAGGCTCTTTCGTGCTCCCTTTTAGACAGGTGATGTAC
GCCCCTTACCCCACCACACACATTGATGTCGACGTGAACACCGTGAAACA
GATGCCTCCATGTCACGAGCACATCTATAACCAGAGAAGATACATGCGGT
CCGAGCTGACCGCTTTCTGGCGGGCCACAAGCGAAGAGGACATGGCTCAG
GACACAATCATCTACACTGATGAGTCCTTCACCCCTGATCTGAACATCTT
CCAAGATGTGCTGCACAGGGACACCCTGGTGAAGGCCTTCCTGGATCAGG
TCTTTCAGCTGAAGCCTGGCCTGTCCCTGCGCTCCACCTTCCTGGCCCAA
TTTCTGCTCGTGCTGCACAGAAAGGCCCTGACCCTGATTAAGTACATCGA
GGACGATACCCAGAAGGGCAAGAAGCCTTTCAAGTCCCTGCGGAATCTGA
AGATCGACCTGGACCTGACCGCCGAGGGCGATCTGAACATCATCATGGCC
CTGGCCGAGAAGATCAAGCCCGGCCTCCACAGCTTCATCTTCGGCAGACC
TTTCTACACCAGCGTGCAGGAGAGAGATGTGCTGATGACATTTTGA

According to some embodiments, the codon optimized sequence is at least 85%, at least 90%, at least 95%, at least 96%, at least 97%, at least 98% or at least 99% identical to SEQ ID NO: 43.

According to some embodiments, the codon optimized sequence comprises SEQ ID NO: 44, shown below.

SEQ ID NO: 44
ATGTCTACACTGTGTCCTCCACCTAGCCCCGCCGTGGCCAAGACAGAAAT
CGCCCTGAGCGGAAAGTCCCCTCTGCTGGCCGCCACATTTGCCTACTGGG
ACAACATACTGGGCCCTAGAGTGCGGCACATCTGGGCCCCTAAGACCGAG
CAGGTGCTGCTGAGCGACGGCGAGATCACCTTCCTGGCCAACCACACCCT
GAACGGCGAAATCCTGAGAAACGCCGAAAGCGGCGCCATCGACGTGAAGT
TCTTCGTGCTGAGCGAGAAAGGCGTGATCATCGTGTCCCTGATCTTCGAC
GGCAACTGGAACGGCGATAGAAGCACCTACGGCCTGAGCATCATTCTGCC
TCAGACCGAGCTGAGCTTCTACCTGCCTCTTCATAGAGTGTGCGTGGACA
GACTGACCCACATTATTAGAAAGGGAAGAATCTGGATGCACAAGGAACGG
CAGGAGAACGTGCAGAAAATCATCCTGGAAGGGACCGAGCGGATGGAAGA
TCAGGGCCAGAGCATCATCCCCATGCTGACAGGCGAGGTGATCCCTGTGA
TGGAACTGCTGTCCAGCATGAAGTCTCACAGCGTGCCCGAGGAAATCGAT
ATCGCCGATACAGTGCTGAACGACGATGACATCGGCGACAGCTGCCACGA
GGGCTTCCTGCTGAATGCCATTTCTAGCCACCTGCAGACATGCGGATGTA
GCGTCGTGGTGGGCTCTAGCGCCGAGAAGGTGAACAAGATCGTGCGGACC
CTGTGCCTGTTCCTGACACCTGCTGAACGCAAGTGCAGCAGACTGTGTGA
AGCCGAAAGCTCTTTTAAGTACGAGAGCGGCCTCTTCGTCCAGGGCCTGC
TGAAGGACAGCACCGGCTCTTTTGTGCTGCCCTTCAGACAGGTGATGTAC
GCCCCTTACCCCACCACCCACATCGACGTCGACGTGAATACCGTGAAACA
GATGCCTCCTTGCCACGAGCACATCTACAACCAGAGAAGATACATGAGAA
GCGAGCTGACAGCCTTCTGGCGGGCCACCTCTGAAGAGGATATGGCCCAG
GACACAATCATCTACACCGACGAGAGCTTCACCCCTGATCTGAACATCTT
CCAAGACGTGCTGCACAGAGATACCCTGGTGAAGGCTTTTCTGGACCAGG
TTTTCCAGCTGAAGCCTGGACTGTCTCTGAGATCTACCTTCCTTGCTCAA
TTTCTGCTGGTCCTCCACCGGAAAGCCCTGACACTGATCAAGTACATCGA
GGACGACACCCAGAAGGGCAAGAAGCCCTTCAAGAGCCTGAGGAACCTGA
AAATCGACCTGGATCTGACCGCCGAGGGCGACCTGAACATCATCATGGCC
CTGGCTGAAAAGATCAAGCCTGGCCTGCACAGTTTCATCTTCGGCAGACC
TTTCTACACCAGCGTGCAGGAGCGGGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 44.

According to some embodiments, the codon optimized sequence comprises SEQ ID NO: 45, shown below.

SEQ ID NO: 45
ATGAGCACCCTGTGCCCCCCCCCCAGCCCCGCCGTGGCCAAGACCGAGAT
CGCCCTGTCTGGCAAGTCCCCTCTGCTTGCCGCTACCTTCGCCTACTGGG
ACAACATCCTGGGCCCTAGAGTGCGGCACATCTGGGCCCCTAAGACCGAG
CAGGTCCTGCTGAGCGACGGCGAAATCACCTTCCTGGCCAACCACACCCT
GAACGGCGAGATCCTGCGGAACGCCGAGAGCGGCGCCATCGACGTGAAGT
TCTTCGTGCTGAGCGAGAAGGGCGTGATCATCGTGTCCCTGATCTTCGAC
GGAAATTGGAACGGCGACAGATCCACATACGGCCTGAGCATCATCCTGCC
TCAGACAGAGCTGTCCTTTTACCTGCCCCTGCACCGGGTGTGCGTGGATA
GACTGACACACATCATTAGAAAGGGAAGAATCTGGATGCACAAGGAACGG
CAGGAGAACGTGCAGAAAATCATCCTGGAAGGTACAGAGAGAATGGAAGA
TCAGGGACAGTCTATCATCCCCATGCTGACCGGCGAGGTGATCCCCGTGA
TGGAACTGCTGAGTTCTATGAAGTCCCACAGCGTGCCTGAGGAAATCGAC
ATCGCCGACACCGTGCTGAACGACGATGACATAGGAGATAGCTGCCACGA
GGGCTTCCTGCTGAATGCCATAAGCAGCCACCTGCAGACCTGTGGCTGCA
GCGTCGTGGTGGGCAGCAGCGCCGAAAAGGTGAACAAGATCGTTAGAACA
CTGTGCCTGTTTCTGACCCCTGCTGAGCGGAAGTGCAGCAGACTGTGTGA
AGCCGAGTCTAGCTTCAAGTACGAGTCCGGCCTGTTCGTGCAAGGCCTGC
TCAAGGACAGCACAGGCTCCTTCGTGCTGCCTTTTAGACAGGTGATGTAC
GCCCCTTACCCCACCACCCATATCGACGTGGACGTGAACACCGTCAAGCA
GATGCCTCCATGTCACGAGCACATCTACAACCAGCGTAGATACATGAGAA
GCGAGCTTACAGCTTTCTGGCGGGCCACCTCTGAAGAGGACATGGCCCAG
GACACCATCATCTACACCGACGAGAGCTTCACCCCTGACCTGAACATTTT
TCAAGATGTGCTGCACAGAGATACCCTGGTGAAAGCCTTCCTGGATCAGG
TGTTCCAGCTGAAACCTGGACTGAGCCTGAGAAGCACCTTCTTGGCACAG
TTCCTCCTGGTCCTGCACAGAAAGGCCCTGACCCTCATCAAGTACATCGA
GGATGATACCCAGAAGGGCAAAAAGCCCTTCAAGAGCCTGAGAAACCTGA
AGATCGATCTGGACCTGACAGCCGAGGGCGACCTGAACATCATCATGGCT
CTGGCTGAAAAAATCAAGCCTGGCCTGCATAGCTTCATCTTCGGCAGACC
TTTCTATACAAGCGTGCAGGAGCGGGACGTGCTGATGACATTCTGA

According to some embodiments, the codon optimized sequence is at least 85%, at least 90%, at least 95%, at least 96%, at least 97%, at least 98% or at least 99% identical to SEQ ID NO: 45.

According to some embodiments, the codon optimized sequence comprises SEQ ID NO: 46, shown below.

SEQ ID NO: 46
ATGAGCACACTGTGTCCTCCTCCGAGCCCTGCTGTGGCCAAGACCGAGAT
CGCCCTGAGCGGCAAGTCCCCACTCCTGGCTGCTACATTCGCCTACTGGG
ACAACATCCTGGGCCCTAGAGTGCGGCACATCTGGGCCCCCAAGACAGAA
CAGGTTCTGCTGAGTGATGGCGAGATCACCTTCCTCGCCAATCACACCCT
GAACGGCGAAATCCTGAGAAACGCCGAGAGCGGCGCCATCGATGTGAAAT
TCTTCGTGCTGAGCGAGAAGGGCGTGATCATCGTGTCCCTGATCTTCGAC
GGCAACTGGAACGGCGATAGAAGCACCTACGGCCTGAGCATCATCCTGCC
CCAGACCGAGCTGAGCTTCTACCTGCCTCTGCACCGGGTGTGCGTGGACA
GACTGACACACATCATTAGAAAGGGCAGAATCTGGATGCACAAGGAACGG
CAGGAGAACGTGCAAAAGATCATTCTGGAAGGGACCGAGCGGATGGAAGA
TCAGGGCCAGAGCATCATCCCTATGCTGACAGGAGAAGTGATCCCCGTGA
TGGAACTGCTGTCTAGCATGAAATCTCACAGCGTGCCCGAGGAAATCGAC
ATCGCCGACACCGTGCTGAACGACGACGACATCGGCGACAGCTGCCATGA
GGGCTTCCTTCTCAACGCCATCAGCAGCCACCTGCAGACCTGTGGCTGCA
GCGTGGTGGTCGGATCTTCTGCCGAAAAGGTGAACAAGATCGTGCGGACC
CTGTGCCTGTTCCTGACCCCTGCCGAACGGAAGTGCAGCAGACTGTGCGA
GGCCGAGAGCAGCTTTAAGTACGAGTCTGGCCTGTTCGTGCAGGGCCTGC
TGAAGGACAGCACAGGCAGCTTTGTGCTGCCTTTTAGACAGGTGATGTAC
GCCCCTTACCCCACCACCCACATCGACGTCGACGTGAACACCGTGAAGCA
GATGCCTCCATGTCACGAGCACATCTACAACCAGCGGAGATACATGAGAT
CCGAGCTGACAGCCTTCTGGCGGGCCACCAGCGAAGAGGATATGGCCCAG
GATACAATCATCTATACAGACGAGTCCTTCACCCCTGATCTGAACATCTT
TCAGGACGTTCTGCACAGAGATACCCTGGTGAAGGCTTTCCTGGACCAAG
TGTTCCAGCTGAAACCTGGACTGAGCCTGCGGAGCACCTTTCTGGCCCAG
TTCCTGCTGGTCCTGCACAGAAAGGCCCTGACCCTGATCAAGTACATCGA
GGACGATACCCAGAAAGGCAAAAAGCCTTTCAAGAGCCTGAGAAATCTGA
AGATCGACCTGGATCTGACCGCCGAGGGAGATCTGAATATCATCATGGCC
CTGGCCGAGAAAATCAAGCCCGGCCTCCATTCTTTCATCTTCGGCAGACC
CTTCTACACATCTGTGCAGGAGCGCGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 46.

According to some embodiments, the codon optimized sequence comprises SEQ ID NO: 47, shown below.

SEQ ID NO: 47
ATGAGCACCCTGTGTCCTCCACCCAGCCCTGCCGTGGCCAAGACAGAGAT
CGCCCTGTCTGGAAAGAGCCCCCTGCTGGCCGCTACCTTCGCCTACTGGG
ACAACATCCTGGGCCCTAGAGTGCGGCACATCTGGGCCCCTAAGACAGAG
CAGGTCCTGCTGAGCGACGGCGAAATCACCTTCCTGGCTAATCACACCCT
TAATGGAGAAATCCTGAGAAACGCCGAATCCGGCGCCATCGACGTGAAGT
TCTTCGTGCTGAGCGAGAAAGGCGTGATCATCGTGTCCCTGATCTTTGAT
GGAAATTGGAACGGCGACAGAAGCACATACGGCCTGAGCATCATCCTGCC
TCAGACCGAGCTGTCTTTTTACCTGCCTCTGCACAGAGTGTGCGTGGACC
GGCTGACCCACATCATCAGAAAGGGCAGAATCTGGATGCACAAGGAACGG
CAGGAGAACGTGCAGAAAATCATTCTGGAAGGCACCGAGCGGATGGAAGA
TCAGGGCCAGAGCATCATCCCCATGCTGACCGGCGAGGTGATCCCCGTGA
TGGAACTGCTGTCTAGCATGAAATCTCACTCTGTGCCTGAGGAAATCGAC
ATCGCCGACACAGTGCTGAACGACGACGACATCGGCGATAGCTGCCACGA
GGGCTTCCTGCTGAACGCCATCAGCAGCCACCTGCAGACATGCGGCTGCA
GCGTGGTCGTGGGAAGCAGCGCCGAAAAGGTGAACAAGATCGTGCGGACC
CTCTGTCTGTTCCTGACGCCCGCCGAGAGAAAGTGCAGCAGACTGTGTGA
AGCCGAGAGCAGCTTTAAGTACGAGTCTGGCCTGTTTGTGCAGGGCCTGC
TGAAGGACAGCACCGGCTCTTTCGTGCTGCCCTTCAGACAGGTGATGTAC
GCCCCTTACCCCACCACACACATTGACGTGGACGTCAACACCGTGAAACA
GATGCCTCCTTGCCATGAACACATCTACAACCAGCGGAGATACATGCGGA
GCGAGCTGACCGCCTTCTGGCGGGCCACCTCTGAGGAAGATATGGCCCAG
GACACCATCATCTATACAGACGAGTCCTTCACCCCTGATCTGAATATCTT
CCAAGATGTTCTCCACAGGGACACCCTGGTGAAGGCTTTTCTCGACCAGG
TGTTCCAGCTGAAACCTGGCCTGAGCCTGCGGAGCACCTTTCTGGCCCAA
TTTCTGCTCGTGCTGCACAGAAAGGCCCTGACCCTGATCAAATACATCGA
GGACGATACACAGAAGGGCAAGAAGCCTTTCAAGTCCCTGAGAAACCTGA
AGATCGACCTGGATCTGACAGCCGAGGGCGACCTGAACATCATTATGGCT
CTGGCCGAGAAGATCAAGCCTGGACTCCACAGCTTCATCTTCGGCCGCCC
CTTCTACACCAGCGTGCAAGAGAGAGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 47.

According to some embodiments, the codon optimized sequence comprises SEQ ID NO: 48. shown below.

SEQ ID NO: 48
ATGAGCACACTGTGCCCCCCCCCTTCTCCTGCCGTGGCCAAGACCGAGAT
TGCCCTGTCCGGCAAGTCCCCTCTGTTGGCCGCCACATTTGCCTACTGGG
ACAACATCCTGGGCCCTAGAGTGCGGCACATTTGGGCCCCTAAGACAGAA
CAGGTGCTGCTGAGTGATGGCGAGATCACCTTTCTGGCCAACCACACCCT
GAATGGCGAAATCCTGAGAAACGCCGAGAGCGGAGCCATCGACGTGAAGT
TCTTCGTGCTGTCTGAGAAGGGTGTTATCATCGTGTCCCTGATCTTCGAC
GGCAACTGGAACGGCGACAGATCTACCTACGGCCTTTCTATCATCCTGCC
CCAGACCGAGCTGAGCTTCTACCTGCCTCTGCATCGGGTGTGCGTGGACC
GGCTGACACACATCATTAGAAAGGGGAGAATCTGGATGCACAAGGAACGC
CAGGAGAACGTGCAGAAAATCATTCTGGAAGGGACCGAAAGAATGGAAGA
TCAGGGCCAGAGCATCATCCCTATGCTGACAGGAGAGGTGATCCCCGTGA
TGGAACTGCTTAGCAGCATGAAGTCTCACAGCGTGCCCGAGGAAATCGAC
ATCGCCGACACCGTGCTGAACGACGACGATATCGGCGACTCATGCCACGA
GGGCTTCCTGCTGAATGCCATCAGCAGCCACCTGCAGACATGCGGCTGTT
CTGTGGTGGTGGGCTCAAGCGCCGAGAAGGTGAACAAGATCGTGCGGACC
CTGTGCCTGTTCCTGACACCTGCTGAGCGGAAGTGCAGCAGACTGTGTGA
AGCCGAATCCAGCTTTAAGTACGAGTCTGGCCTCTTCGTGCAAGGCCTGC
TGAAGGACAGCACCGGCTCTTTTGTGCTGCCTTTTAGACAGGTGATGTAC
GCCCCTTACCCCACCACACACATCGACGTTGATGTCAACACCGTGAAACA
GATGCCTCCATGTCACGAGCACATCTACAACCAGAGAAGATACATGAGAA
GCGAGCTGACCGCCTTTTGGCGGGCCACCAGCGAGGAAGATATGGCCCAG
GACACCATCATCTATACCGACGAGTCCTTCACCCCTGATCTGAACATCTT
CCAAGACGTGCTGCACCGGGACACACTGGTCAAGGCCTTCCTGGACCAAG
TGTTCCAGCTGAAGCCCGGCCTGAGCCTGCGGAGCACCTTCCTGGCTCAG
TTCCTGCTGGTGCTTCACCGGAAGGCCCTGACCCTTATCAAGTACATCGA
GGACGACACCCAGAAGGGCAAAAAGCCTTTCAAGAGCCTGAGAAATCTGA
AAATCGACCTGGATCTGACAGCCGAAGGCGATCTGAACATCATCATGGCC
CTTGCTGAGAAAATCAAGCCAGGCCTGCACAGCTTTATCTTCGGCAGACC
TTTCTACACCAGCGTGCAGGAGAGAGATGTGCTGATGACCTTCTGA

According to some embodiments, the codon optimized sequence is at least 85%, at least 90%, at least 95%, at least 96%, at least 97%, at least 98% or at least 99% identical to SEQ ID NO: 48.

According to some embodiments, the codon optimized sequence comprises SEQ ID NO: 49, shown below.

SEQ ID NO: 49
ATGAGCACCCTCTGTCCTCCTCCATCTCCTGCCGTGGCAAAGACCGAGAT
CGCCCTGTCCGGCAAAAGCCCCCTGCTGGCCGCTACATTCGCCTACTGGG
ACAACATCCTCGGACCTAGAGTGCGGCACATCTGGGCCCCTAAGACCGAG
CAGGTTCTGCTGAGCGACGGCGAGATAACATTTCTGGCCAACCACACCCT
GAACGGCGAGATCCTGAGAAACGCCGAGAGCGGCGCCATCGATGTGAAGT
TCTTCGTGCTCTCTGAGAAGGGCGTGATCATTGTGTCCCTGATCTTCGAC
GGCAACTGGAACGGCGATAGATCCACCTACGGCCTGAGCATCATCCTGCC
CCAGACAGAGCTGTCTTTTTACCTGCCTCTGCACCGGGTGTGCGTGGACA
GACTGACACACATCATCAGAAAGGGCAGAATCTGGATGCACAAGGAACGG
CAGGAGAACGTGCAGAAAATCATCCTGGAAGGCACCGAGAGAATGGAAGA
TCAGGGCCAGAGCATCATTCCTATGCTGACTGGAGAGGTGATCCCCGTGA
TGGAACTGCTGTCTAGCATGAAAAGCCACAGCGTGCCCGAGGAAATCGAC
ATCGCCGACACCGTGCTGAACGACGACGACATCGGCGACAGCTGCCACGA
GGGCTTCCTGCTCAATGCCATCAGCTCCCACCTGCAGACATGCGGCTGCA
GCGTGGTCGTGGGCAGCAGCGCCGAAAAGGTGAACAAGATCGTGCGGACA
CTGTGTCTGTTCCTGACCCCTGCTGAAAGAAAGTGCAGCAGACTGTGCGA
GGCCGAATCTAGCTTTAAGTACGAGAGCGGCCTCTTCGTGCAAGGCCTGC
TGAAGGACTCCACAGGCAGCTTCGTGCTGCCTTTTAGACAGGTGATGTAC
GCCCCTTATCCTACAACCCACATCGACGTGGACGTCAATACCGTGAAGCA
GATGCCTCCATGTCACGAGCACATCTACAACCAGAGAAGATACATGAGAA
GCGAGCTGACCGCTTTTTGGCGGGCCACAAGCGAGGAAGATATGGCCCAG
GACACCATCATCTATACTGATGAGTCTTTCACCCCTGATCTGAACATCTT
CCAAGATGTGCTCCATAGAGATACCCTGGTCAAAGCCTTCCTGGACCAGG
TGTTCCAGCTGAAACCCGGCCTGAGCCTGAGATCTACCTTCCTGGCTCAG
TTCCTGCTGGTGCTGCACAGAAAGGCCCTGACCCTGATCAAGTACATCGA
GGATGATACCCAGAAGGGAAAAAAGCCCTTCAAGTCCCTGCGGAACCTGA
AGATCGACCTGGATCTGACCGCCGAGGGCGACCTGAATATCATCATGGCC
CTGGCCGAAAAGATCAAGCCAGGACTGCATAGCTTCATCTTCGGCAGACC
TTTCTACACATCTGTGCAGGAGCGGGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 49.

According to some embodiments, the codon optimized sequence comprises SEQ ID NO: 50, shown below.

SEQ ID NO: 50
ATGAGCACACTCTGTCCTCCTCCGAGCCCAGCCGTGGCAAAGACCGAGAT
CGCCCTGTCTGGCAAGTCCCCTCTGCTGGCCGCCACCTTCGCCTACTGGG
ACAACATCCTGGGCCCTAGAGTGCGGCACATCTGGGCCCCTAAGACCGAG
CAGGTGCTGCTGAGCGACGGAGAAATCACCTTCCTGGCTAATCACACCCT
GAACGGCGAGATCCTGCGGAACGCCGAAAGCGGCGCCATCGACGTGAAGT
TCTTCGTGCTGAGCGAGAAGGGAGTGATCATCGTGTCCCTGATCTTCGAC
GGCAACTGGAACGGCGACCGATCTACATACGGCCTGAGCATCATCCTGCC
ACAGACAGAGCTGAGCTTTTACCTGCCCCTGCATAGAGTGTGCGTGGACA
GACTGACCCACATCATTAGAAAGGGCAGAATCTGGATGCACAAGGAACGG
CAGGAGAACGTGCAAAAGATCATCCTGGAAGGCACCGAAAGAATGGAAGA
TCAGGGCCAGAGCATCATTCCTATGCTGACCGGCGAGGTGATCCCCGTGA
TGGAACTGTTGTCCAGCATGAAATCTCACAGCGTCCCCGAGGAAATCGAC
ATCGCCGACACCGTGCTGAACGACGACGATATCGGCGACTCATGCCATGA
GGGATTCCTGCTGAATGCCATCAGCAGCCACCTGCAGACCTGCGGCTGTA
GCGTGGTCGTGGGCAGCAGTGCCGAGAAGGTGAACAAGATCGTGCGGACC
CTGTGTCTGTTTCTGACCCCTGCCGAAAGAAAGTGCAGCAGACTGTGCGA
GGCCGAGAGCAGCTTCAAGTACGAGTCTGGCCTGTTCGTGCAGGGCCTGC
TGAAAGACAGCACCGGATCTTTCGTGCTGCCTTTTAGACAGGTGATGTAC
GCCCCTTATCCTACAACCCACATTGACGTCGACGTCAACACCGTGAAACA
GATGCCTCCGTGCCACGAGCACATCTACAACCAGAGGCGGTACATGAGAT
CTGAGCTGACAGCCTTCTGGCGGGCCACAAGCGAAGAGGACATGGCCCAG
GACACCATCATCTACACTGATGAGAGCTTCACCCCTGATCTGAACATCTT
CCAAGACGTGCTGCACCGGGACACCCTGGTCAAGGCCTTTCTCGACCAGG
TGTTCCAGCTGAAGCCCGGCCTGTCCCTGAGATCCACATTTCTTGCTCAG
TTCCTGCTGGTGCTGCACAGAAAAGCCCTGACACTGATCAAGTACATCGA
GGACGACACACAGAAGGGCAAAAAGCCTTTCAAAAGCCTGAGAAACCTGA
AGATCGATCTGGACCTGACCGCCGAGGGCGATCTTAATATCATCATGGCC
CTGGCCGAAAAAATCAAGCCTGGCCTGCACTCTTTTATCTTCGGCAGACC
TTTCTACACCAGCGTGCAGGAGAGAGATGTGCTGATGACCTTCTGA

According to some embodiments, the codon optimized sequence is at least 85%, at least 90%, at least 95%, at least 96%, at least 97%, at least 98% or at least 99% identical to SEQ ID NO: 50.

According to some embodiments, the codon optimized sequence comprises SEQ ID NO: 51, shown below.

SEQ ID NO: 51
ATGAGCACCCTCTGCCCCCCCCCCAGCCCCGCCGTGGCCAAGACAGAAAT
CGCCCTGTCTGGCAAGTCCCCTCTGCTGGCCGCCACCTTTGCCTACTGGG
ACAACATCCTGGGCCCTAGAGTGCGGCACATCTGGGCCCCTAAGACCGAG
CAAGTGCTGCTGTCTGATGGAGAAATCACCTTCCTGGCTAATCACACACT
GAACGGCGAGATCCTGCGGAACGCCGAGTCTGGAGCCATCGACGTGAAAT
TCTTCGTGCTGAGCGAGAAGGGCGTGATCATCGTGTCCCTGATCTTCGAC
GGCAACTGGAACGGCGATAGAAGCACCTACGGCCTGTCCATCATCCTGCC
TCAGACAGAGCTGTCCTTCTACCTGCCACTGCACCGGGTGTGCGTGGACA
GACTGACCCACATTATTAGAAAGGGCAGAATCTGGATGCACAAGGAACGG
CAGGAGAACGTGCAGAAGATCATTCTGGAAGGGACCGAGAGAATGGAAGA
TCAGGGCCAGAGCATCATCCCTATGCTGACTGGCGAGGTGATCCCCGTGA
TGGAACTGCTGAGCTCCATGAAAAGCCATTCTGTCCCCGAGGAAATCGAC
ATCGCCGACACCGTGCTGAACGACGACGATATCGGCGACAGCTGCCACGA
GGGCTTCCTGCTGAATGCCATCAGCTCTCATCTGCAGACCTGCGGCTGCA
GCGTCGTGGTGGGCTCTAGCGCCGAGAAGGTGAACAAGATCGTGCGGACA
CTGTGCCTGTTCCTGACACCTGCCGAGAGGAAGTGCAGCAGACTGTGTGA
AGCCGAATCTAGCTTTAAGTACGAGAGCGGCCTGTTCGTGCAAGGCCTGC
TGAAGGACAGCACAGGCAGCTTCGTGCTGCCTTTCAGACAGGTGATGTAC
GCCCCTTACCCCACCACCCACATCGATGTTGACGTGAACACCGTGAAGCA
GATGCCTCCATGTCACGAGCACATCTACAACCAGCGGAGATACATGCGGA
GCGAGCTGACCGCCTTTTGGCGGGCCACAAGCGAAGAGGACATGGCTCAG
GACACAATCATCTACACTGATGAGAGCTTCACCCCTGATCTGAACATTTT
CCAAGACGTGCTCCACAGAGATACCCTGGTGAAGGCCTTCCTGGACCAGG
TTTTCCAGCTGAAACCTGGACTGAGCCTGAGAAGCACCTTCCTGGCCCAG
TTCCTGCTCGTGCTGCACAGAAAGGCCCTGACCCTTATCAAGTATATCGA
GGACGACACCCAGAAAGGCAAAAAGCCCTTCAAGAGCCTGAGAAACCTGA
AGATCGACCTGGATCTGACCGCCGAGGGAGATCTGAACATCATCATGGCC
CTGGCCGAGAAAATCAAGCCTGGCCTGCACAGCTTTATCTTCGGCCGCCC
CTTTTACACAAGCGTGCAGGAGAGAGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 51.

According to some embodiments, the codon optimized sequence comprises SEQ ID NO: 52, shown below.

SEQ ID NO: 52
ATGAGCACACTGTGTCCTCCTCCTAGCCCCGCCGTGGCCAAGACCGAGAT
CGCCCTCAGCGGCAAGTCTCCACTGCTCGCCGCTACCTTCGCCTACTGGG
ACAACATCCTGGGCCCTAGAGTGCGGCACATCTGGGCCCCTAAGACAGAG
CAGGTCCTTCTGAGCGACGGCGAGATAACATTCCTGGCCAACCACACACT
GAACGGCGAGATCCTCAGGAACGCCGAATCTGGCGCCATCGACGTGAAGT
TCTTCGTGCTGTCTGAGAAGGGCGTGATTATTGTGTCCCTGATCTTCGAC
GGAAATTGGAACGGCGACCGGAGCACATACGGCCTGTCCATCATCCTGCC
CCAGACGGAACTGTCTTTTTACCTGCCTCTGCACAGAGTGTGCGTGGACA
GACTGACCCACATCATTAGAAAGGGCAGAATCTGGATGCACAAGGAAAGA
CAGGAGAACGTGCAGAAAATCATCCTGGAAGGTACAGAGAGAATGGAAGA
TCAGGGACAGAGCATCATCCCTATGCTGACTGGCGAAGTGATCCCCGTGA
TGGAACTGCTGTCCAGCATGAAAAGCCACAGCGTGCCCGAGGAAATCGAC
ATCGCCGACACTGTGCTGAACGACGATGATATCGGCGACAGCTGCCATGA
GGGCTTCCTGCTGAATGCCATCAGCTCTCACCTGCAGACCTGTGGATGTA
GCGTGGTGGTCGGCAGCAGCGCCGAAAAGGTGAACAAGATTGTGCGGACC
CTGTGCCTGTTCCTCACACCTGCTGAGAGAAAGTGCAGCAGACTGTGCGA
GGCCGAGAGCAGCTTCAAGTACGAGAGCGGCCTGTTCGTGCAGGGCCTGC
TGAAGGACAGCACCGGCTCCTTCGTTCTGCCTTTCCGGCAGGTGATGTAC
GCCCCTTACCCCACCACCCACATCGATGTTGACGTGAATACCGTGAAACA
GATGCCTCCATGTCACGAGCACATCTACAACCAGAGAAGATACATGAGAA
GCGAGCTGACCGCCTTCTGGCGGGCCACCAGCGAAGAGGACATGGCCCAG
GACACCATCATCTACACCGACGAGAGCTTCACCCCTGATCTGAACATCTT
TCAGGATGTGCTCCATAGAGATACCCTGGTCAAGGCCTTCCTGGACCAGG
TGTTCCAGCTGAAACCTGGACTGAGCCTGCGCAGCACCTTCCTGGCTCAA
TTTCTACTTGTGCTGCACCGGAAGGCCCTGACACTGATCAAGTACATCGA
GGACGACACCCAGAAGGGCAAAAAGCCCTTTAAGAGCCTGAGAAACCTGA
AGATCGACCTGGATCTGACAGCCGAAGGCGATCTGAACATCATCATGGCT
CTTGCTGAGAAAATCAAGCCAGGACTGCATTCTTTCATCTTCGGCCGCCC
CTTCTACACATCTGTGCAGGAGCGGGACGTGCTGATGACCTTCTGA

According to some embodiments, the codon optimized sequence is at least 85%, at least 90%, at least 95%, at least 96%, at least 97%, at least 98% or at least 99% identical to SEQ ID NO: 52.

Gene Structure of Multiplexed Expression of c9orf72 with Artificial Intron (A.I.)

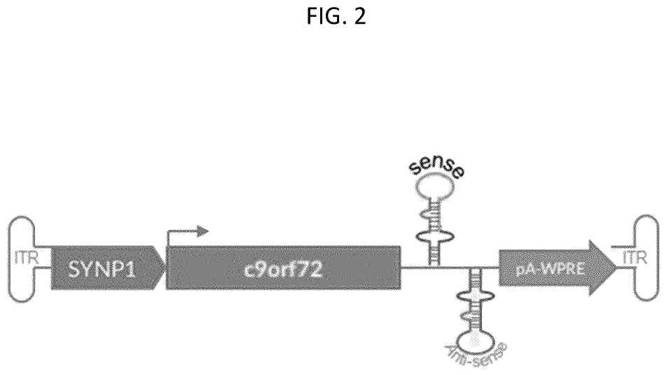

The gene structure of c9orf72-AI (artificial intron) is shown in FIG. 1A. The corresponding nucleic acid sequence is shown in FIG. 1B. The artificial structures for c9orf72 supplementation are shown in FIG. 2. A customer designed artificial intron harboring His-cMyc tags and His-HA tags were added for v1 and v3 transcript, respectively. The A.I. sequence was tested in vitro using plasmid transfection.

Final AAV Construct Size

The final size of the AAV construct is about 4.8 kb. The promoters employed for the final AAV version were: a hSyn promoter (neuron specific), a CBA promoter (ubiquitous), or a CASI promoter (ubiquitous).

Multi-Variant (v1-NM-145005 & v2-NM-018325) c9orf72 Supplementation

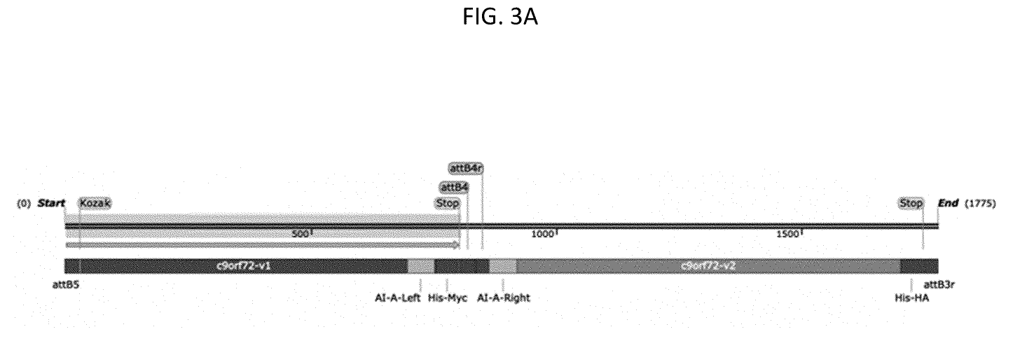

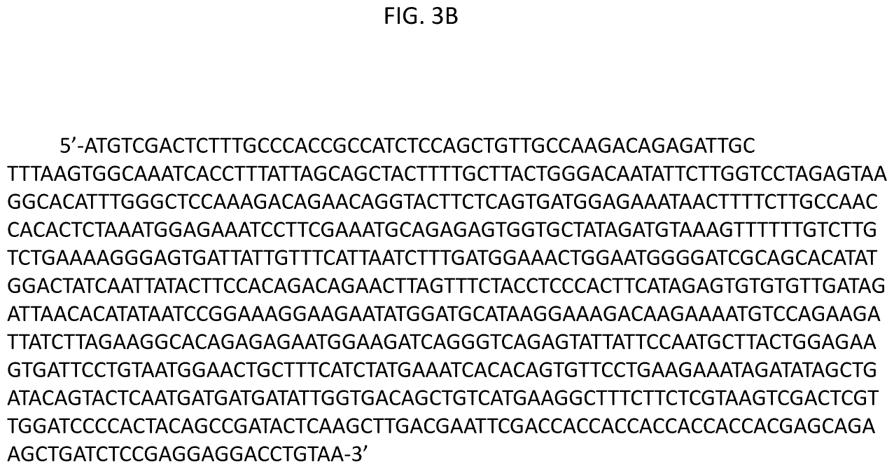

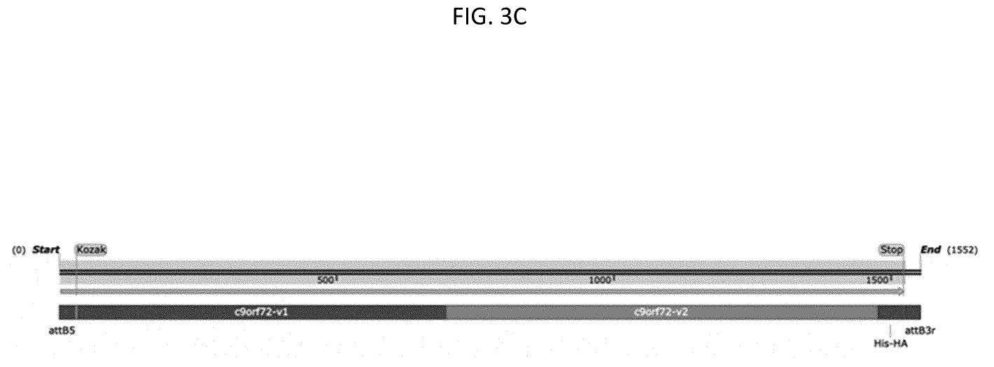

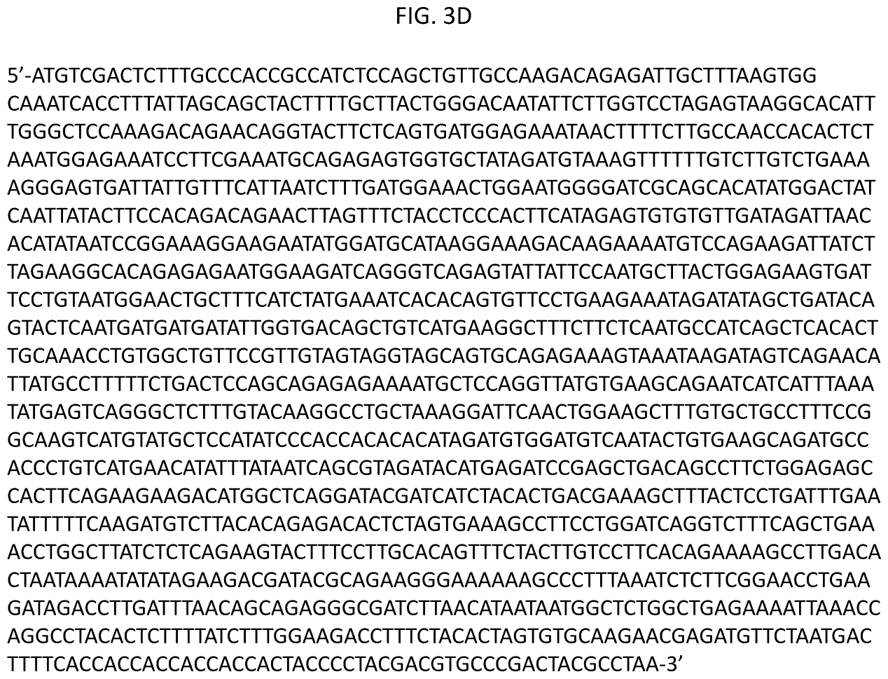

Wildtype (WT) cells express predominantly v1 (NM-145005) & v2 (NM-018325). An “Alternative Stop-or-Go” design was proposed for v1 & v2 cistronic variants. The splicing efficiency of artificial “intron” was found to be less than 100%. The v1 variant came from translation read-through on non-spliced mRNA. The v2 variant came from spliced mRNA. The ratio of v1/v2 was balanced by changing artificial intron properties. Schematic constructs of alternative translation are shown in FIGS. 3A-3D. FIG. 3A is a schematic showing the first open reading frame of an alternative translation of c9orf72. FIG. 3B shows the corresponding nucleic acid sequence. FIG. 3C is a schematic showing the second open reading frame after splicing of an alternative translation of c9orf72. FIG. 3D shows the corresponding nucleic acid sequence.

Experimental Design Validating Cistronic v1 & v2 Supplementation

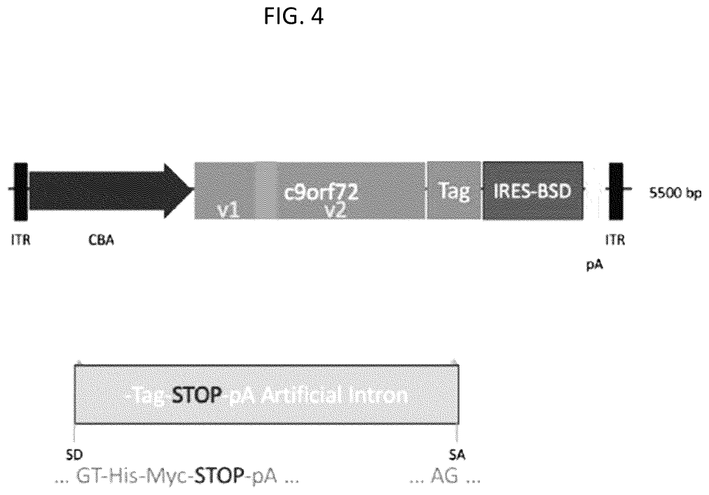

The testing construct carried BSD or Puro element as selection marker. BSD: blasticidin resistant to ensure v1 & v2 expression ratio measure. Blasticidin resistance ensures non-transduced cells expressing WT c9orf72 variants will die off. Therefore, recombinant v1 vs v2 ratio was measured. The final AAV construct did not include the BSD marker. FIG. 4 shows a schematic of constructs with selection marker.

The Following Multi-Variant c9orf72 Constructs were Prepared:

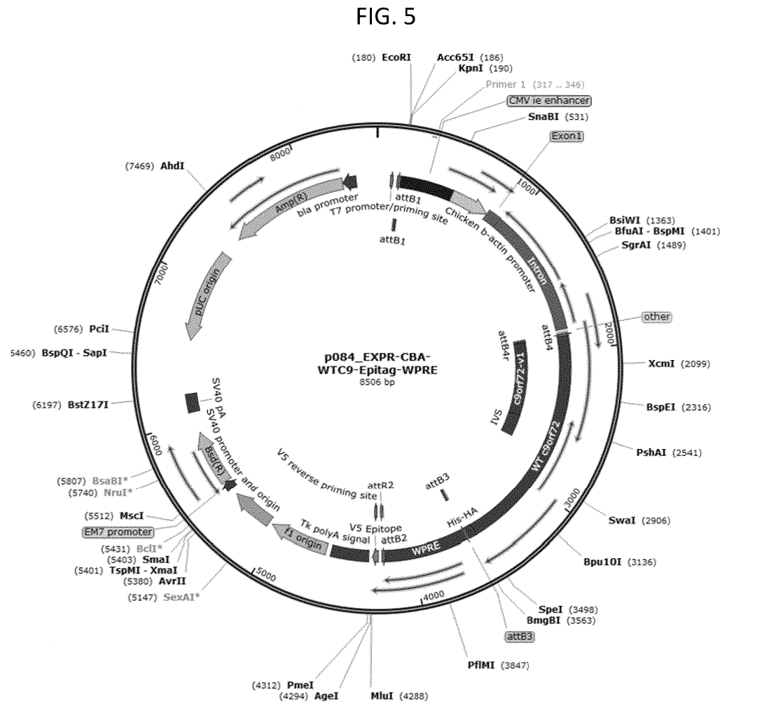

(1) p084_EXPR_pcDNA_CBA_WTC9-EpiTag_WPRE. This construct comprises CBA promoter, wildtype C9orf72 sequence (long isoform) tagged with His and HA tag, TK polyA signal. Ampicillin resistance gene. The vector map is shown in FIG. 5. According to some embodiments, the nucleic acid sequence of p084_EXPR_pcDNA_CBA_WTC9-EpiTag_WPRE comprises SEQ ID NO: 53. According to some embodiments, the nucleic acid sequence of p084_EXPR_pcDNA_CBA_WTC9-EpiTag_WPRE is at least 85%, at least 90%, at least 95%, at least 96%, at least 97%, at least 98%, at least 99% identical to SEQ ID NO: 53, shown below.

agtgcgcgagcaaaatttaagctacaacaaggcaaggcttgaccgacaattctctggctaacta
gagaacccactgcttactggcttatcgaaattaatacgactcactatagggagacccaagctgg
ctagttaagctatcaacaagtttGTACAAAAAAGCAGGCTTActcagatctgaattcggtacct
agttattaatagtaatcaattacggggtcattagttcatagcccatatatggagttccgcgtta
cataacttacggtaaatggcccgcctggctgaccgcccaacgacccccgcccattgacgtcaat
aatgacgtatgttcccatagtaacgccaatagggactttccattgacgtcaatgggtggagtat
ttacggtaaactgcccacttggcagtacatcaagtgtatcatatgccaagtacgccccctattg
acgtcaatgacggtaaatggcccgcctggcattatgcccagtacatgaccttatgggactttcc
tacttggcagtacatctacgtattagtcatcgctattaccatggtcgaggtgagccccacgttc
tgcttcactctccccatctcccccccctccccacccccaattttgtatttatttattttttaat
tattttgtgcagcgatgggggcggggggggggggggggcgcgcgccaggcggggcggggcgggg
cgaggggcggggcggggcgaggcggagaggtgcggcggcagccaatcagagcggcgcgctccga
aagtttccttttatggcgaggcggcggcggcggcggccctataaaaagcgaagcgcgcggcggg
cgggagtcgctgcgcgctgccttcgccccgtgccccgctccgccgccgcctcgcgccgcccgcc
ccggctctgactgaccgcgttactcccacaggtgagcgggcgggacggcccttctcctccgggc
tgtaattagcgcttggtttaatgacggcttgtttcttttctgtggctgcgtgaaagccttgagg
ggctccgggagggccctttgtgcggggggagcggctcggggggtgcgtgcgtgtgtgtgtgcgt
ggggagcgccgcgtgcggctccgcgctgcccggcggctgtgagcgctgcgggcgcggcgcgggg
ctttgtgcgctccgcagtgtgcgcgaggggagcgcggccgggggcggtgccccgcggtgcgggg
ggggctgcgaggggaacaaaggctgcgtgcggggtgtgtgcgtgggggggtgagcagggggtgt
gggcgcgtcggtcgggctgcaaccccccctgcacccccctccccgagttgctgagcacggcccg
gcttcgggtgcggggctccgtacggggcgtggcgcggggctcgccgtgccgggcggggggtggc
ggcaggtgggggtgccgggcggggcggggccgcctcgggccggggagggctcgggggaggggcg
cggcggcccccggagcgccggcggctgtcgaggcgcggcgagccgcagccattgccttttatgg
taatcgtgcgagagggcgcagggacttcctttgtcccaaatctgtgcggagccgaaatctggga
ggcgccgccgcaccccctctagcgggcgcggggcgaagcggtgcggcgccggcaggaaggaaat
gggcggggagggccttcgtgcgtcgccgcgccgccgtccccttctccctctccagcctcggggc
tgtccgcggggggacggctgccttcgggggggacggggcagggcggggttcggcttctggcgtg
tgaccggcggctctagagcctctgctaaccatgttcatgccttcttctttttcctacagctcct
gggcaacgccaccatggCACCCAACTTTTCTATACAAAGTTGTAATGTCGACTCTTTGCCCACC
GCCATCTCCAGCTGTTGCCAAGACAGAGATTGCTTTAAGTGGCAAATCACCTTTATTAGCAGCT
ACTTTTGCTTACTGGGACAATATTCTTGGTCCTAGAGTAAGGCACATTTGGGCTCCAAAGACAG
AACAGGTACTTCTCAGTGATGGAGAAATAACTTTTCTTGCCAACCACACTCTAAATGGAGAAAT
CCTTCGAAATGCAGAGAGTGGTGCTATAGATGTAAAGTTTTTTGTCTTGTCTGAAAAGGGAGTG
ATTATTGTTTCATTAATCTTTGATGGAAACTGGAATGGGGATCGCAGCACATATGGACTATCAA
TTATACTTCCACAGACAGAACTTAGTTTCTACCTCCCACTTCATAGAGTGTGTGTTGATAGATT
AACACATATAATCCGGAAAGGAAGAATATGGATGCATAAGGAAAGACAAGAAAATGTCCAGAAG
ATTATCTTAGAAGGCACAGAGAGAATGGAAGATCAGGGTCAGAGTATTATTCCAATGCTTACTG
GAGAAGTGATTCCTGTAATGGAACTGCTTTCATCTATGAAATCACACAGTGTTCCTGAAGAAAT
AGATATAGCTGATACAGTACTCAATGATGATGATATTGGTGACAGCTGTCATGAAGGCTTTCTT
CTCgtaagtCACCACCACCACCACCACGAGCAGAAGCTGATCTCCGAGGAGGACCTGTAAatca
aggttacaagacaggAATAAAtttaaggagaccaatagaaactgggcttgtcgagacagagaag
actcttgcgtttctgataggcacctattggtcttactgacatccactttgcctttctctccaca
gAATGCCATCAGCTCACACTTGCAAACCTGTGGCTGTTCCGTTGTAGTAGGTAGCAGTGCAGAG
AAAGTAAATAAGATAGTCAGAACATTATGCCTTTTTCTGACTCCAGCAGAGAGAAAATGCTCCA
GGTTATGTGAAGCAGAATCATCATTTAAATATGAGTCAGGGCTCTTTGTACAAGGCCTGCTAAA
GGATTCAACTGGAAGCTTTGTGCTGCCTTTCCGGCAAGTCATGTATGCTCCATATCCCACCACA
CACATAGATGTGGATGTCAATACTGTGAAGCAGATGCCACCCTGTCATGAACATATTTATAATC
AGCGTAGATACATGAGATCCGAGCTGACAGCCTTCTGGAGAGCCACTTCAGAAGAAGACATGGC
TCAGGATACGATCATCTACACTGACGAAAGCTTTACTCCTGATTTGAATATTTTTCAAGATGTC
TTACACAGAGACACTCTAGTGAAAGCCTTCCTGGATCAGGTCTTTCAGCTGAAACCTGGCTTAT
CTCTCAGAAGTACTTTCCTTGCACAGTTTCTACTTGTCCTTCACAGAAAAGCCTTGACACTAAT
AAAATATATAGAAGACGATACGCAGAAGGGAAAAAAGCCCTTTAAATCTCTTCGGAACCTGAAG
ATAGACCTTGATTTAACAGCAGAGGGCGATCTTAACATAATAATGGCTCTGGCTGAGAAAATTA
AACCAGGCCTACACTCTTTTATCTTTGGAAGACCTTTCTACACTAGTGTGCAAGAACGAGATGT
TCTAATGACTTTTCACCACCACCACCACCACTACCCCTACGACGTGCCCGACTACGCCTAAACA
ACTTTGTATAATAAAGTTGTAaatcaacctctggattacaaaatttgtgaaagattgactggta
ttcttaactatgttgctccttttacgctatgtggatacgctgctttaatgcctttgtatcatgc
tattgcttcccgtatggctttcattttctcctccttgtataaatcctggttgctgtctctttat
gaggagttgtggcccgttgtcaggcaacgtggcgtggtgtgcactgtgtttgctgacgcaaccc
ccactggttggggcattgccaccacctgtcagctcctttccgggactttcgctttccccctccc
tattgccacggcggaactcatcgccgcctgccttgcccgctgctggacaggggctcggctgttg
ggcactgacaattccgtggtgttgtcggggaaatcatcgtcctttccttggctgctcgcctgtg
ttgccacctggattctgcgcgggacgtccttctgctacgtcccttcggccctcaatccagcgga
ccttccttcccgcggcctgctgccggctctgcggcctcttccgcgtcttcgccttcgccctcag
acgagtcggatctccctttgggccgcctccccgcctgAACCCAGCTTTcttgtacaaagtggtt
gatctagagggcccgcggttcgaaggtaagcctatccctaaccctctcctcggtctcgattcta
cgcgtaccggttagtaatgagtttaaacgggggaggctaactgaaacacggaaggagacaatac
cggaaggaacccgcgctatgacggcaataaaaagacagaataaaacgcacgggtgttgggtcgt
ttgttcataaacgcggggttcggtcccagggctggcactctgtcgataccccaccgagacccca
ttggggccaatacgcccgcgtttcttccttttccccaccccaccccccaagttcgggtgaaggc
ccagggctcgcagccaacgtcggggcggcaggccctgccatagcagatctgcgcagctggggct
ctagggggtatccccacgcgccctgtagcggcgcattaagcgcggcgggtgtggtggttacgcg
cagcgtgaccgctacacttgccagcgccctagcgcccgctcctttcgctttcttcccttccttt
ctcgccacgttcgccggctttccccgtcaagctctaaatcgggggctccctttagggttccgat
ttagtgctttacggcacctcgaccccaaaaaacttgattagggtgatggttcacgtagtgggcc
atcgccctgatagacggtttttcgccctttgacgttggagtccacgttctttaatagtggactc
ttgttccaaactggaacaacactcaaccctatctcggtctattcttttgatttataagggattt
tgccgatttcggcctattggttaaaaaatgagctgatttaacaaaaatttaacgcgaattaatt
ctgtggaatgtgtgtcagttagggtgtggaaagtccccaggctccccagcaggcagaagtatgc
aaagcatgcatctcaattagtcagcaaccaggtgtggaaagtccccaggctccccagcaggcag
aagtatgcaaagcatgcatctcaattagtcagcaaccatagtcccgcccctaactccgcccatc
ccgcccctaactccgcccagttccgcccattctccgccccatggctgactaattttttttattt
atgcagaggccgaggccgcctctgcctctgagctattccagaagtagtgaggaggcttttttgg
aggcctaggcttttgcaaaaagctcccgggagcttgtatatccattttcggatctgatcagcac
gtgttgacaattaatcatcggcatagtatatcggcatagtataatacgacaaggtgaggaacta
aaccatggccaagcctttgtctcaagaagaatccaccctcattgaaagagcaacggctacaatc
aacagcatccccatctctgaagactacagcgtcgccagcgcagctctctctagcgacggccgca
tcttcactggtgtcaatgtatatcattttactgggggaccttgtgcagaactcgtggtgctggg
cactgctgctgctgcggcagctggcaacctgacttgtatcgtcgcgatcggaaatgagaacagg
ggcatcttgagcccctgcggacggtgccgacaggtgcttctcgatctgcatcctgggatcaaag
ccatagtgaaggacagtgatggacagccgacggcagttgggattcgtgaattgctgccctctgg
ttatgtgtgggagggctaagcacttcgtggccgaggagcaggactgacacgtgctacgagattt
cgattccaccgccgccttctatgaaaggttgggcttcggaatcgttttccgggacgccggctgg
atgatcctccagcgcggggatctcatgctggagttcttcgcccaccccaacttgtttattgcag
cttataatggttacaaataaagcaatagcatcacaaatttcacaaataaagcatttttttcact
gcattctagttgtggtttgtccaaactcatcaatgtatcttatcatgtctgtataccgtcgacc
tctagctagagcttggcgtaatcatggtcatagctgtttcctgtgtgaaattgttatccgctca
caattccacacaacatacgagccggaagcataaagtgtaaagcctggggtgcctaatgagtgag
ctaactcacattaattgcgttgcgctcactgcccgctttccagtcgggaaacctgtcgtgccag
ctgcattaatgaatcggccaacgcgcggggagaggcggtttgcgtattgggcgctcttccgctt
cctcgctcactgactcgctgcgctcggtcgttcggctgcggcgagcggtatcagctcactcaaa
ggcggtaatacggttatccacagaatcaggggataacgcaggaaagaacatgtgagcaaaaggc
cagcaaaaggccaggaaccgtaaaaaggccgcgttgctggcgtttttccataggctccgccccc
ctgacgagcatcacaaaaatcgacgctcaagtcagaggtggcgaaacccgacaggactataaag
ataccaggcgtttccccctggaagctccctcgtgcgctctcctgttccgaccctgccgcttacc
ggatacctgtccgcctttctcccttcgggaagcgtggcgctttctcatagctcacgctgtaggt
atctcagttcggtgtaggtcgttcgctccaagctgggctgtgtgcacgaaccccccgttcagcc
cgaccgctgcgccttatccggtaactatcgtcttgagtccaacccggtaagacacgacttatcg
ccactggcagcagccactggtaacaggattagcagagcgaggtatgtaggcggtgctacagagt
tcttgaagtggtggcctaactacggctacactagaagaacagtatttggtatctgcgctctgct
gaagccagttaccttcggaaaaagagttggtagctcttgatccggcaaacaaaccaccgctggt
agcggtggtttttttgtttgcaagcagcagattacgcgcagaaaaaaaggatctcaagaagatc
ctttgatcttttctacggggtctgacgctcagtggaacgaaaactcacgttaagggattttggt
catgagattatcaaaaaggatcttcacctagatccttttaaattaaaaatgaagttttaaatca
atctaaagtatatatgagtaaacttggtctgacagttaccaatgcttaatcagtgaggcaccta
tctcagcgatctgtctatttcgttcatccatagttgcctgactccccgtcgtgtagataactac
gatacgggagggcttaccatctggccccagtgctgcaatgataccgcgagacccacgctcaccg
gctccagatttatcagcaataaaccagccagccggaagggccgagcgcagaagtggtcctgcaa
ctttatccgcctccatccagtctattaattgttgccgggaagctagagtaagtagttcgccagt
taatagtttgcgcaacgttgttgccattgctacaggcatcgtggtgtcacgctcgtcgtttggt
atggcttcattcagctccggttcccaacgatcaaggcgagttacatgatcccccatgttgtgca
aaaaagcggttagctccttcggtcctccgatcgttgtcagaagtaagttggccgcagtgttatc
actcatggttatggcagcactgcataattctcttactgtcatgccatccgtaagatgcttttct
gtgactggtgagtactcaaccaagtcattctgagaatagtgtatgcggcgaccgagttgctctt
gcccggcgtcaatacgggataataccgcgccacatagcagaactttaaaagtgctcatcattgg
aaaacgttcttcggggcgaaaactctcaaggatcttaccgctgttgagatccagttcgatgtaa
cccactcgtgcacccaactgatcttcagcatcttttactttcaccagcgtttctgggtgagcaa
aaacaggaaggcaaaatgccgcaaaaaagggaataagggcgacacggaaatgttgaatactcat
actcttcctttttcaatattattgaagcatttatcagggttattgtctcatgagcggatacata
tttgaatgtatttagaaaaataaacaaataggggttccgcgcacatttccccgaaaagtgccac
ctgacgtcgacggatcgggagatctcccgatcccctatggtgcactctcagtacaatctgctct
gatgccgcatagttaagccagtatctgctccctgcttgtgtgttggaggtcgctgagt

According to some embodiments, p084_Expr_pcDNA_CBA_WTC9-EpiTag_WPRE_2-FP-CBA_(forward primer) (1195 bp) comprises SEQ ID NO: 54.

NNNNNNNNNNNCNNNNTGTTCNTGCCTTCTTCTTTTTCCTACAGCTCCTG
GGCAACGCCACCATGGCACCCAACTTTTCTATACAAAGTTGTAATGTCGA
CTCTTTGCCCACCGCCATCTCCAGCTGTTGCCAAGACAGAGATTGCTTTA
AGTGGCAAATCACCTTTATTAGCAGCTACTTTTGCTTACTGGGACAATAT
TCTTGGTCCTAGAGTAAGGCACATTTGGGCTCCAAAGACAGAACAGGTAC
TTCTCAGTGATGGAGAAATAACTTTTCTTGCCAACCACACTCTAAATGGA
GAAATCCTTCGAAATGCAGAGAGTGGTGCTATAGATGTAAAGTTTTTTGT
CTTGTCTGAAAAGGGAGTGATTATTGTTTCATTAATCTTTGATGGAAACT
GGAATGGGGATCGCAGCACATATGGACTATCAATTATACTTCCACAGACA
GAACTTAGTTTCTACCTCCCACTTCATAGAGTGTGTGTTGATAGATTAAC
ACATATAATCCGGAAAGGAAGAATATGGATGCATAAGGAAAGACAAGAAA
ATGTCCAGAAGATTATCTTAGAAGGCACAGAGAGAATGGAAGATCAGGGT
CAGAGTATTATTCCAATGCTTACTGGAGAAGTGATTCCTGTAATGGAACT
GCTTTCATCTATGAAATCACACAGTGTTCCTGAAGAAATAGATATAGCTG
ATACAGTACTCAATGATGATGATATTGGTGACAGCTGTCATGAAGGCTTT
CTTCTCGTAAGTCACCACCACCACCACCACGAGCAGAAGCTGATCTCCGA
GGAGGACCTGTAAATCAAGGTTACAAGACAGGAATAAATTTAAGGAGACC
AATAGAAACTGGGCTTGTCGAGACAGAGAAGACTCTTGCGTTTCTGATAG
GCACCTATTGGNCTTACTGACATCNCTTTGCCTTTCTCTCACAGAATGCA
TCAGCTCACACTTNCAANCNGTGNTGNNCNNNTAGTANNAGCAGTGCANA
GAAGTAAATAGANAGTCNGANNTNNNCTTTTTNCTGANTCNNNNNANNNA
AATGCTCNNNNNNNANCNNNANCATCNTTTANNNNANTCNNNNNNTTGTN
NNGNNGCNAANNTNACTNNNCTNNNNCTNNNNNNANNCANGNNNNNNNNN
NNNNNNNNNNNNNNNNNNNNNNNNNNNNNNNNNNNNNNNNNGNCN

According to some embodiments, p084_Expr_pcDNA_CBA_WTC9-EpiTag_WPRE_2-RP-WPRE_reverse primer (1212 bp) comprises SEQ ID NO: 55.

NNNNNNNNNNATTNAGCAGCGTATCCACATAGCGTAAAGGAGCAACATAG
TTAAGAATACCAGTCAATCTTTCACAAATTTTGTAATCCAGAGGTTGATT
TACAACTTTATTATACAAAGTTGTTTACAGGTCCTCCTCGGAGATCAGCT
TCTGCTCGTGGTGGTGGTGGTGGTGAAAAGTCATTAGAACATCTCGTTCT
TGCACACTAGTGTAGAAAGGTCTTCCAAAGATAAAAGAGTGTAGGCCTGG
TTTAATTTTCTCAGCCAGAGCCATTATTATGTTAAGATCGCCCTCTGCTG
TTAAATCAAGGTCTATCTTCAGGTTCCGAAGAGATTTAAAGGGCTTTTTT
CCCTTCTGCGTATCGTCTTCTATATATTTTATTAGTGTCAAGGCTTTTCT
GTGAAGGACAAGTAGAAACTGTGCAAGGAAAGTACTTCTGAGAGATAAGC
CAGGTTTCAGCTGAAAGACCTGATCCAGGAAGGCTTTCACTAGAGTGTCT
CTGTGTAAGACATCTTGAAAAATATTCAAATCAGGAGTAAAGCTTTCGTC
AGTGTAGATGATCGTATCCTGAGCCATGTCTTCTTCTGAAGTGGCTCTCC
AGAAGGCTGTCAGCTCGGATCTCATGTATCTACGCTGATTATAAATATGT
TCATGACAGGGTGGCATCTGCTTCACAGTATTGACATCCACATCTATGTG
TGTGGTGGGATATGGAGCATACATGACTTGCCGGAAAGGCAGCACAAAGC
TTCCAGTTGAATCCTTTAGCAGGCCTTGTACAAAGAGCCCTGACTCATAT
TTAAATGATGATTCTGCTTCACATAACCTGGNNCATTTTCTCTCTGCTGG
NGTCAGAAAAAGGCATAATGTTCTGACTATCTTATTTACTTTCTCTGCAC
TGCTACCTACTACAACGGANAGCCACAGGTTTGCAAGTGTGAGCTGATGG
CATTCTGTGGAGAGAAAGGCAAAGTGGNTGTCAGTANACCANTAGNGCCT
ATCANAAACGCANAGTCTTCTCTGNNNCGANAGCCANTTTCTNNNNNNNN
NNNAATTNTTNCTGNNNNNNANCTGANTTNNCNNGTCCNCCNNCGNNANA
NTNNNCTNNNNNNNNNNNNNNNNNNNNNNNTNCNANAANNAAAGCNNCNN
NNNNNNCNNTNNNNNNNCNNCNNNNNTGNAGNACNGNNNTCNNNNNNNNN
NNNNNNNNNGNA

(2) p085_EXPR_pcDNA_CASI_WTC9-EpiTag_WPRE. This construct comprises

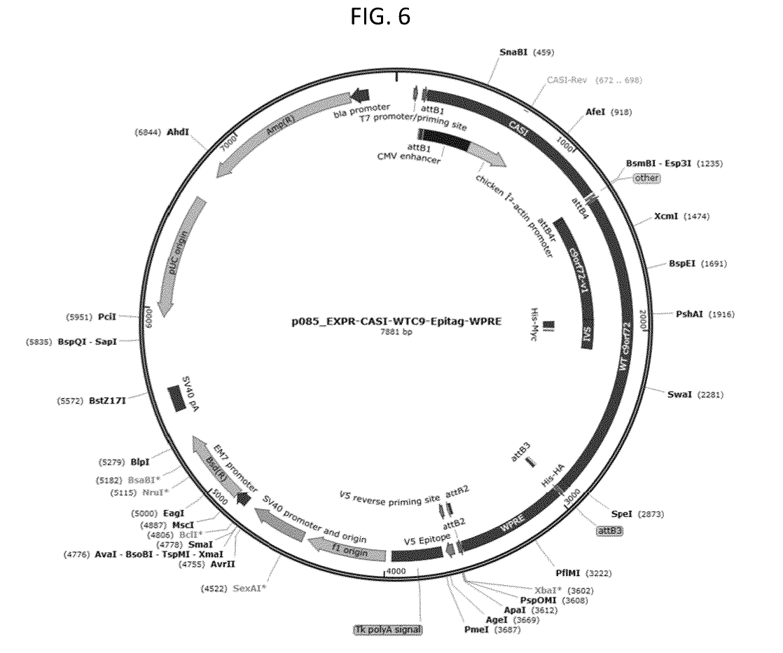

CASI promoter, wildtype C9orf72 sequence (express only long isoform) tagged with His and HA tag, TK polyA signal. Ampicillin resistance gene. The vector map is shown in FIG. 6. According to some embodiments, the nucleic acid sequence of p085_EXPR_pcDNA_CASI_WTC9-EpiTag_WPRE comprises SEQ ID NO:56. According to some embodiments, the nucleic acid sequence of p085_EXPR_pcDNA_CASI_WTC9-EpiTag_WPRE is at least 85%, at least 90%, at least 95%, at least 96%, at least 97%, at least 98%, at least 99% identical to SEQ ID NO: 56, shown below.

agtgcgcgagcaaaatttaagctacaacaaggcaaggcttgaccgacaattctctggctaacta
gagaacccactgcttactggcttatcgaaattaatacgactcactatagggagacccaagctgg
ctagttaagctatcaacaagtttGTACAAAAAAGCAGGCTTAggagttccgcgttacataactt
acggtaaatggcccgcctggctgaccgcccaacgacccccgcccattgacgtcaataatgacgt
atgttcccatagtaacgccaatagggactttccattgacgtcaatgggtggagtatttacggta
aactgcccacttggcagtacatcaagtgtatcatatgccaagtacgccccctattgacgtcaat
gacggtaaatggcccgcctggcattatgcccagtacatgaccttatgggactttcctacttggc
agtacatctacgtattagtcatcgctattaccatggtcgaggtgagccccacgttctgcttcac
tctccccatctcccccccctccccacccccaattttgtatttatttattttttaattattttgt
gcagcgatgggggcgggggggggggggggcgcgcgccaggcggggcggggcggggcgaggggcg
gggcggggcgaggcggagaggtgcggcggcagccaatcagagcggcgcgctccgaaagtttcct
tttatggcgaggcggcggcggcggcggccctataaaaagcgaagcgcgcggcgggcgggagtcg
ctgcgcgctgccttcgccccgtgccccgctccgccgccgcctcgcgccgcccgccccggctctg
actgaccgcgttactaaaacaggtaagtccggcctccgcgccgggttttggcgcctcccgcggg
cgcccccctcctcacggcgagcgctgccacgtcagacgaagggcgcagcgagcgtcctgatcct
tccgcccggacgctcaggacagcggcccgctgctcataagactcggccttagaaccccagtatc
agcagaaggacattttaggacgggacttgggtgactctagggcactggttttctttccagagag
cggaacaggcgaggaaaagtagtcccttctcggcgattctgcggagggatctccgtggggcggt
gaacgccgatgatgcctctactaaccatgttcatgttttctttttttttctacaggtcctgggt
gacgaacagacgcgtctcgaacgccaccatggCACCCAACTTTTCTATACAAAGTTGTAATGTC
GACTCTTTGCCCACCGCCATCTCCAGCTGTTGCCAAGACAGAGATTGCTTTAAGTGGCAAATCA
CCTTTATTAGCAGCTACTTTTGCTTACTGGGACAATATTCTTGGTCCTAGAGTAAGGCACATTT
GGGCTCCAAAGACAGAACAGGTACTTCTCAGTGATGGAGAAATAACTTTTCTTGCCAACCACAC
TCTAAATGGAGAAATCCTTCGAAATGCAGAGAGTGGTGCTATAGATGTAAAGTTTTTTGTCTTG
TCTGAAAAGGGAGTGATTATTGTTTCATTAATCTTTGATGGAAACTGGAATGGGGATCGCAGCA
CATATGGACTATCAATTATACTTCCACAGACAGAACTTAGTTTCTACCTCCCACTTCATAGAGT
GTGTGTTGATAGATTAACACATATAATCCGGAAAGGAAGAATATGGATGCATAAGGAAAGACAA
GAAAATGTCCAGAAGATTATCTTAGAAGGCACAGAGAGAATGGAAGATCAGGGTCAGAGTATTA
TTCCAATGCTTACTGGAGAAGTGATTCCTGTAATGGAACTGCTTTCATCTATGAAATCACACAG
TGTTCCTGAAGAAATAGATATAGCTGATACAGTACTCAATGATGATGATATTGGTGACAGCTGT
CATGAAGGCTTTCTTCTCgtaagtCACCACCACCACCACCACGAGCAGAAGCTGATCTCCGAGG
AGGACCTGTAAatcaaggttacaagacaggAATAAAtttaaggagaccaatagaaactgggctt
gtcgagacagagaagactcttgcgtttctgataggcacctattggtcttactgacatccacttt
gcctttctctccacagAATGCCATCAGCTCACACTTGCAAACCTGTGGCTGTTCCGTTGTAGTA
GGTAGCAGTGCAGAGAAAGTAAATAAGATAGTCAGAACATTATGCCTTTTTCTGACTCCAGCAG
AGAGAAAATGCTCCAGGTTATGTGAAGCAGAATCATCATTTAAATATGAGTCAGGGCTCTTTGT
ACAAGGCCTGCTAAAGGATTCAACTGGAAGCTTTGTGCTGCCTTTCCGGCAAGTCATGTATGCT
CCATATCCCACCACACACATAGATGTGGATGTCAATACTGTGAAGCAGATGCCACCCTGTCATG
AACATATTTATAATCAGCGTAGATACATGAGATCCGAGCTGACAGCCTTCTGGAGAGCCACTTC
AGAAGAAGACATGGCTCAGGATACGATCATCTACACTGACGAAAGCTTTACTCCTGATTTGAAT
ATTTTTCAAGATGTCTTACACAGAGACACTCTAGTGAAAGCCTTCCTGGATCAGGTCTTTCAGC
TGAAACCTGGCTTATCTCTCAGAAGTACTTTCCTTGCACAGTTTCTACTTGTCCTTCACAGAAA
AGCCTTGACACTAATAAAATATATAGAAGACGATACGCAGAAGGGAAAAAAGCCCTTTAAATCT
CTTCGGAACCTGAAGATAGACCTTGATTTAACAGCAGAGGGCGATCTTAACATAATAATGGCTC
TGGCTGAGAAAATTAAACCAGGCCTACACTCTTTTATCTTTGGAAGACCTTTCTACACTAGTGT
GCAAGAACGAGATGTTCTAATGACTTTTCACCACCACCACCACCACTACCCCTACGACGTGCCC
GACTACGCCTAAACAACTTTGTATAATAAAGTTGTAaatcaacctctggattacaaaatttgtg
aaagattgactggtattcttaactatgttgctccttttacgctatgtggatacgctgctttaat
gcctttgtatcatgctattgcttcccgtatggctttcattttctcctccttgtataaatcctgg
ttgctgtctctttatgaggagttgtggcccgttgtcaggcaacgtggcgtggtgtgcactgtgt
ttgctgacgcaacccccactggttggggcattgccaccacctgtcagctcctttccgggacttt
cgctttccccctccctattgccacggcggaactcatcgccgcctgccttgcccgctgctggaca
ggggctcggctgttgggcactgacaattccgtggtgttgtcggggaaatcatcgtcctttcctt
ggctgctcgcctgtgttgccacctggattctgcgcgggacgtccttctgctacgtcccttcggc
cctcaatccagcggaccttccttcccgcggcctgctgccggctctgcggcctcttccgcgtctt
cgccttcgccctcagacgagtcggatctccctttgggccgcctccccgcctgAACCCAGCTTTc
ttgtacaaagtggttgatctagagggcccgcggttcgaaggtaagcctatccctaaccctctcc
tcggtctcgattctacgcgtaccggttagtaatgagtttaaacgggggaggctaactgaaacac
ggaaggagacaataccggaaggaacccgcgctatgacggcaataaaaagacagaataaaacgca
cgggtgttgggtcgtttgttcataaacgcggggttcggtcccagggctggcactctgtcgatac
cccaccgagaccccattggggccaatacgcccgcgtttcttccttttccccaccccacccccca
agttcgggtgaaggcccagggctcgcagccaacgtcggggcggcaggccctgccatagcagatc
tgcgcagctggggctctagggggtatccccacgcgccctgtagcggcgcattaagcgcggcggg
tgtggtggttacgcgcagcgtgaccgctacacttgccagcgccctagcgcccgctcctttcgct
ttcttcccttcctttctcgccacgttcgccggctttccccgtcaagctctaaatcgggggctcc
ctttagggttccgatttagtgctttacggcacctcgaccccaaaaaacttgattagggtgatgg
ttcacgtagtgggccatcgccctgatagacggtttttcgccctttgacgttggagtccacgttc
tttaatagtggactcttgttccaaactggaacaacactcaaccctatctcggtctattcttttg
atttataagggattttgccgatttcggcctattggttaaaaaatgagctgatttaacaaaaatt
taacgcgaattaattctgtggaatgtgtgtcagttagggtgtggaaagtccccaggctccccag
caggcagaagtatgcaaagcatgcatctcaattagtcagcaaccaggtgtggaaagtccccagg
ctccccagcaggcagaagtatgcaaagcatgcatctcaattagtcagcaaccatagtcccgccc
ctaactccgcccatcccgcccctaactccgcccagttccgcccattctccgccccatggctgac
taattttttttatttatgcagaggccgaggccgcctctgcctctgagctattccagaagtagtg
aggaggcttttttggaggcctaggcttttgcaaaaagctcccgggagcttgtatatccattttc
ggatctgatcagcacgtgttgacaattaatcatcggcatagtatatcggcatagtataatacga
caaggtgaggaactaaaccatggccaagcctttgtctcaagaagaatccaccctcattgaaaga
gcaacggctacaatcaacagcatccccatctctgaagactacagcgtcgccagcgcagctctct
ctagcgacggccgcatcttcactggtgtcaatgtatatcattttactgggggaccttgtgcaga
actcgtggtgctgggcactgctgctgctgcggcagctggcaacctgacttgtatcgtcgcgatc
ggaaatgagaacaggggcatcttgagcccctgcggacggtgccgacaggtgcttctcgatctgc
atcctgggatcaaagccatagtgaaggacagtgatggacagccgacggcagttgggattcgtga
attgctgccctctggttatgtgtgggagggctaagcacttcgtggccgaggagcaggactgaca
cgtgctacgagatttcgattccaccgccgccttctatgaaaggttgggcttcggaatcgttttc
cgggacgccggctggatgatcctccagcgcggggatctcatgctggagttcttcgcccacccca
acttgtttattgcagcttataatggttacaaataaagcaatagcatcacaaatttcacaaataa
agcatttttttcactgcattctagttgtggtttgtccaaactcatcaatgtatcttatcatgtc
tgtataccgtcgacctctagctagagcttggcgtaatcatggtcatagctgtttcctgtgtgaa
attgttatccgctcacaattccacacaacatacgagccggaagcataaagtgtaaagcctgggg
tgcctaatgagtgagctaactcacattaattgcgttgcgctcactgcccgctttccagtcggga
aacctgtcgtgccagctgcattaatgaatcggccaacgcgcggggagaggcggtttgcgtattg
ggcgctcttccgcttcctcgctcactgactcgctgcgctcggtcgttcggctgcggcgagcggt
atcagctcactcaaaggcggtaatacggttatccacagaatcaggggataacgcaggaaagaac
atgtgagcaaaaggccagcaaaaggccaggaaccgtaaaaaggccgcgttgctggcgtttttcc
ataggctccgcccccctgacgagcatcacaaaaatcgacgctcaagtcagaggtggcgaaaccc
gacaggactataaagataccaggcgtttccccctggaagctccctcgtgcgctctcctgttccg
accctgccgcttaccggatacctgtccgcctttctcccttcgggaagcgtggcgctttctcata
gctcacgctgtaggtatctcagttcggtgtaggtcgttcgctccaagctgggctgtgtgcacga
accccccgttcagcccgaccgctgcgccttatccggtaactatcgtcttgagtccaacccggta
agacacgacttatcgccactggcagcagccactggtaacaggattagcagagcgaggtatgtag
gcggtgctacagagttcttgaagtggtggcctaactacggctacactagaagaacagtatttgg
tatctgcgctctgctgaagccagttaccttcggaaaaagagttggtagctcttgatccggcaaa
caaaccaccgctggtagcggtggtttttttgtttgcaagcagcagattacgcgcagaaaaaaag
gatctcaagaagatcctttgatcttttctacggggtctgacgctcagtggaacgaaaactcacg
ttaagggattttggtcatgagattatcaaaaaggatcttcacctagatccttttaaattaaaaa
tgaagttttaaatcaatctaaagtatatatgagtaaacttggtctgacagttaccaatgcttaa
tcagtgaggcacctatctcagcgatctgtctatttcgttcatccatagttgcctgactccccgt
cgtgtagataactacgatacgggagggcttaccatctggccccagtgctgcaatgataccgcga
gacccacgctcaccggctccagatttatcagcaataaaccagccagccggaagggccgagcgca
gaagtggtcctgcaactttatccgcctccatccagtctattaattgttgccgggaagctagagt
aagtagttcgccagttaatagtttgcgcaacgttgttgccattgctacaggcatcgtggtgtca
cgctcgtcgtttggtatggcttcattcagctccggttcccaacgatcaaggcgagttacatgat
cccccatgttgtgcaaaaaagcggttagctccttcggtcctccgatcgttgtcagaagtaagtt
ggccgcagtgttatcactcatggttatggcagcactgcataattctcttactgtcatgccatcc
gtaagatgcttttctgtgactggtgagtactcaaccaagtcattctgagaatagtgtatgcggc
gaccgagttgctcttgcccggcgtcaatacgggataataccgcgccacatagcagaactttaaa
agtgctcatcattggaaaacgttcttcggggcgaaaactctcaaggatcttaccgctgttgaga
tccagttcgatgtaacccactcgtgcacccaactgatcttcagcatcttttactttcaccagcg
tttctgggtgagcaaaaacaggaaggcaaaatgccgcaaaaaagggaataagggcgacacggaa
atgttgaatactcatactcttcctttttcaatattattgaagcatttatcagggttattgtctc
atgagcggatacatatttgaatgtatttagaaaaataaacaaataggggttccgcgcacatttc
cccgaaaagtgccacctgacgtcgacggatcgggagatctcccgatcccctatggtgcactctc
agtacaatctgctctgatgccgcatagttaagccagtatctgctccctgcttgtgtgttggagg
tcgctgagt

According to some embodiments, p085_Expr_pcDNA_CASI_WTC9-EpiTag_WPRE_6-RP-WPRE-01 (1164 bp) comprises SEQ ID NO: 57, shown below.

NNNNNNNNNNATTAAGCAGCGTATCCACATAGCGTAAAGGAGCAACATAG
TTAAGAATACCAGTCAATCTTTCACAAATTTTGTAATCCAGAGGTTGATT
TACAACTTTATTATACAAAGTTGTTTACAGGTCCTCCTCGGAGATCAGCT
TCTGCTCGTGGTGGTGGTGGTGGTGAAAAGTCATTAGAACATCTCGTTCT
TGCACACTAGTGTAGAAAGGTCTTCCAAAGATAAAAGAGTGTAGGCCTGG
TTTAATTTTCTCAGCCAGAGCCATTATTATGTTAAGATCGCCCTCTGCTG
TTAAATCAAGGTCTATCTTCAGGTTCCGAAGAGATTTAAAGGGCTTTTTT
CCCTTCTGCGTATCGTCTTCTATATATTTTATTAGTGTCAAGGCTTTTCT
GTGAAGGACAAGTAGAAACTGTGCAAGGAAAGTACTTCTGAGAGATAAGC
CAGGTTTCAGCTGAAAGACCTGATCCAGGAAGGCTTTCACTAGAGTGTCT
CTGTGTAAGACATCTTGAAAAATATTCAAATCAGGAGTAAAGCTTTCGTC
AGTGTAGATGATCGTATCCTGAGCCATGTCTTCTTCTGAAGTGGCTCTCC
AGAAGGCTGTCAGCTCGGATCTCATGTATCTACGCTGATTATAAATATGT
TCATGACAGGGTGGCATCTGCTTCACAGTATTGACATCCACATCTATGTG
TGTGGTGGGATATGGAGCATACATGACTTGCCGGAAAGGCAGCACAAAGC
TTCCAGTTGAATCCTTTAGCAGGCCTTGTACAAAGAGCCCTGACTCATAT
TTAAATGATGATTCTGCTTCACATAACCTGGNGCATTTTCTCTCTGCTGG
AGTCAGAAAAAGGCATAATGTTCTGACTATCTTATTTACTTTCTCTGCAC
TGCTACCTACTACACGGANAGCNCAGGTTTGCAGTGTGAGCTGATGGCAT
TCTGTGNGAGAANGNAAGTNNNGTCAGTANNNNNNGNNCNATCANNNNNA
GANTCTTCTCTGNNTNGANANCCNNTTNCNNTNNNNNNNAANNNNNGTCT
GNACTGATTNNNGNCNNCNNNGNNNNTCAGCTNCNGNNNNNGNNNGNNGN
NNNNNNTNCNANANNNAANNCNTNNNGNNNCNNTNNNCNNNNTCATNCNN
NNNNNNANNACNNN

According to some embodiments, p085_Expr_pcDNA_CASI_WTC9-EpiTag_WPRE_6-FP-CASI (1162 bp) comprises SEQ ID NO: 58, shown below.

NNNNNNNNNNNNGGTNNNGCCGATGATGCCTCTACTAACCATGTTCATGT
TTTCTTTTTTTTTCTACAGGTCCTGGGTGACGAACAGACGCGTCTCGAAC
GCCACCATGGCACCCAACTTTTCTATACAAAGTTGTAATGTCGACTCTTT
GCCCACCGCCATCTCCAGCTGTTGCCAAGACAGAGATTGCTTTAAGTGGC
AAATCACCTTTATTAGCAGCTACTTTTGCTTACTGGGACAATATTCTTGG
TCCTAGAGTAAGGCACATTTGGGCTCCAAAGACAGAACAGGTACTTCTCA
GTGATGGAGAAATAACTTTTCTTGCCAACCACACTCTAAATGGAGAAATC
CTTCGAAATGCAGAGAGTGGTGCTATAGATGTAAAGTTTTTTGTCTTGTC
TGAAAAGGGAGTGATTATTGTTTCATTAATCTTTGATGGAAACTGGAATG
GGGATCGCAGCACATATGGACTATCAATTATACTTCCACAGACAGAACTT
AGTTTCTACCTCCCACTTCATAGAGTGTGTGTTGATAGATTAACACATAT
AATCCGGAAAGGAAGAATATGGATGCATAAGGAAAGACAAGAAAATGTCC
AGAAGATTATCTTAGAAGGCACAGAGAGAATGGAAGATCAGGGTCAGAGT
ATTATTCCAATGCTTACTGGAGAAGTGATTCCTGTAATGGAACTGCTTTC
ATCTATGAAATCACACAGTGTTCCTGAAGAAATAGATATAGCTGATACAG
TACTCAATGATGATGATATTGGTGACAGCTGTCATGAAGGCTTTCTTCTC
GTAAGTCACCACCACCACCACCACGAGCAGAAGCTGATCTCCGAGGAGGA
CCTGTAAATCAAGGGTTACAAGACAGGAATAAATTTAAGGAGACCAATAG
AAACTGGGCTTGTCGAGACNGANANACTCTTGCGTTTCTGATAGGCANCT
ATTGNNTNCTGACATCCACTTTGCCTTTCTCTCNCAGANGCNTCAGCTCA
CACTNNAANCTGNGNTNNNNNNNAGTAGNAGCAGTGCNNANAAGTAANNA
GANAGTCNNANNTNNNCNTTTTNCTGACTNCNNCNNNNNNAATGCTCNNN
NANNNNAAGNNANCNTCNNNNNNNNANTCNNNNNNTTNNACNNNNNNCTA
AANGNANTNNNN

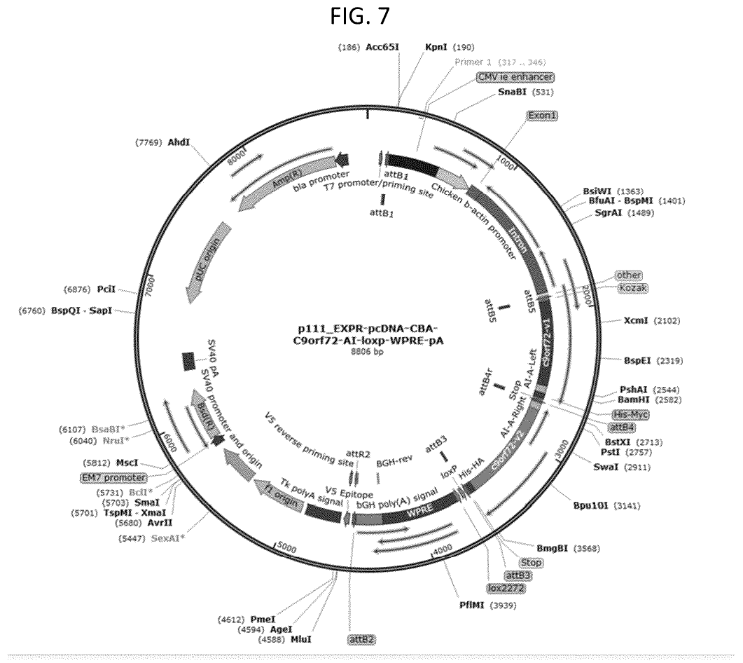

(3) p111_EXPR-pcDNA-CBA-C9orf72-AI-loxp-WPRE-pA. This construct comprises CBA promoter, polyA signal, Ampicillin resistance gene. This construct carry a C9orf72 sequence designed to express long C9orf72 protein isoform tagged with His and HA, a short C90rf72 protein isoform tagged with His and Myc tag. The vector map is shown in FIG. 7. According to some embodiments, the nucleic acid sequence of p111_EXPR-pcDNA-CBA-C9orf72-AI-loxp-WPRE-pA comprises SEQ ID NO: 59. According to some embodiments, the nucleic acid sequence of p111_EXPR-pcDNA-CBA-C9orf72-AI-loxp-WPRE-pA is at least 85%, at least 90%, at least 95%, at least 96%, at least 97%, at least 98%, at least 99% identical to SEQ ID NO: 59, shown below.

agtgcgcgagcaaaatttaagctacaacaaggcaaggcttgaccgacaattctctggctaacta
gagaacccactgcttactggcttatcgaaattaatacgactcactatagggagacccaagctgg
ctagttaagctatcaacaagtttGTACAAAAAAGCAGGCTTActcagatctgaattcggtacct
agttattaatagtaatcaattacggggtcattagttcatagcccatatatggagttccgcgtta
cataacttacggtaaatggcccgcctggctgaccgcccaacgacccccgcccattgacgtcaat
aatgacgtatgttcccatagtaacgccaatagggactttccattgacgtcaatgggtggagtat
ttacggtaaactgcccacttggcagtacatcaagtgtatcatatgccaagtacgccccctattg
acgtcaatgacggtaaatggcccgcctggcattatgcccagtacatgaccttatgggactttcc
tacttggcagtacatctacgtattagtcatcgctattaccatggtcgaggtgagccccacgttc
tgcttcactctccccatctcccccccctccccacccccaattttgtatttatttattttttaat
tattttgtgcagcgatgggggcggggggggggggggggcgcgcgccaggcggggcggggcgggg
cgaggggcggggcggggcgaggcggagaggtgcggcggcagccaatcagagcggcgcgctccga
aagtttccttttatggcgaggcggcggcggcggcggccctataaaaagcgaagcgcgcggcggg
cgggagtcgctgcgcgctgccttcgccccgtgccccgctccgccgccgcctcgcgccgcccgcc
ccggctctgactgaccgcgttactcccacaggtgagcgggcgggacggcccttctcctccgggc
tgtaattagcgcttggtttaatgacggcttgtttcttttctgtggctgcgtgaaagccttgagg
ggctccgggagggccctttgtgcggggggagcggctcggggggtgcgtgcgtgtgtgtgtgcgt
ggggagcgccgcgtgcggctccgcgctgcccggcggctgtgagcgctgcgggcgcggcgcgggg
ctttgtgcgctccgcagtgtgcgcgaggggagcgcggccgggggcggtgccccgcggtgcgggg
ggggctgcgaggggaacaaaggctgcgtgcggggtgtgtgcgtgggggggtgagcagggggtgt
gggcgcgtcggtcgggctgcaaccccccctgcacccccctccccgagttgctgagcacggcccg
gcttcgggtgcggggctccgtacggggcgtggcgcggggctcgccgtgccgggcggggggtggc
ggcaggtgggggtgccgggcggggcggggccgcctcgggccggggagggctcgggggaggggcg
cggcggcccccggagcgccggcggctgtcgaggcgcggcgagccgcagccattgccttttatgg
taatcgtgcgagagggcgcagggacttcctttgtcccaaatctgtgcggagccgaaatctggga
ggcgccgccgcaccccctctagcgggcgcggggcgaagcggtgcggcgccggcaggaaggaaat
gggcggggagggccttcgtgcgtcgccgcgccgccgtccccttctccctctccagcctcggggc
tgtccgcggggggacggctgccttcgggggggacggggcagggcggggttcggcttctggcgtg
tgaccggcggctctagagcctctgctaaccatgttcatgccttcttctttttcctacagctcct
gggcaacgccaccatggACAACTTTGTATACAAAAGTTGTAgccaccATGTCGACTCTTTGCCC
ACCGCCATCTCCAGCTGTTGCCAAGACAGAGATTGCTTTAAGTGGCAAATCACCTTTATTAGCA
GCTACTTTTGCTTACTGGGACAATATTCTTGGTCCTAGAGTAAGGCACATTTGGGCTCCAAAGA
CAGAACAGGTACTTCTCAGTGATGGAGAAATAACTTTTCTTGCCAACCACACTCTAAATGGAGA
AATCCTTCGAAATGCAGAGAGTGGTGCTATAGATGTAAAGTTTTTTGTCTTGTCTGAAAAGGGA
GTGATTATTGTTTCATTAATCTTTGATGGAAACTGGAATGGGGATCGCAGCACATATGGACTAT
CAATTATACTTCCACAGACAGAACTTAGTTTCTACCTCCCACTTCATAGAGTGTGTGTTGATAG
ATTAACACATATAATCCGGAAAGGAAGAATATGGATGCATAAGGAAAGACAAGAAAATGTCCAG
AAGATTATCTTAGAAGGCACAGAGAGAATGGAAGATCAGGGTCAGAGTATTATTCCAATGCTTA
CTGGAGAAGTGATTCCTGTAATGGAACTGCTTTCATCTATGAAATCACACAGTGTTCCTGAAGA
AATAGATATAGCTGATACAGTACTCAATGATGATGATATTGGTGACAGCTGTCATGAAGGCTTT
CTTCTCgtaagtcgactcgttggatccccactacagccgatactcaagcttgacgaattcgacC
ACCACCACCACCACCACGAGCAGAAGCTGATCTCCGAGGAGGACCTGTAACACCCAACTTTTCT
ATACAAAGTTGTAgtatccaaggtagtggactagtgtgacgctgctgacccctttctttccctt
ctgcagAATGCCATCAGCTCACACTTGCAAACCTGTGGCTGTTCCGTTGTAGTAGGTAGCAGTG
CAGAGAAAGTAAATAAGATAGTCAGAACATTATGCCTTTTTCTGACTCCAGCAGAGAGAAAATG
CTCCAGGTTATGTGAAGCAGAATCATCATTTAAATATGAGTCAGGGCTCTTTGTACAAGGCCTG
CTAAAGGATTCAACTGGAAGCTTTGTGCTGCCTTTCCGGCAAGTCATGTATGCTCCATATCCCA
CCACACACATAGATGTGGATGTCAATACTGTGAAGCAGATGCCACCCTGTCATGAACATATTTA
TAATCAGCGTAGATACATGAGATCCGAGCTGACAGCCTTCTGGAGAGCCACTTCAGAAGAAGAC
ATGGCTCAGGATACGATCATCTACACTGACGAAAGCTTTACTCCTGATTTGAATATTTTTCAAG
ATGTCTTACACAGAGACACTCTAGTGAAAGCCTTCCTGGATCAGGTCTTTCAGCTGAAACCTGG
CTTATCTCTCAGAAGTACTTTCCTTGCACAGTTTCTACTTGTCCTTCACAGAAAAGCCTTGACA
CTAATAAAATATATAGAAGACGATACGCAGAAGGGAAAAAAGCCCTTTAAATCTCTTCGGAACC
TGAAGATAGACCTTGATTTAACAGCAGAGGGCGATCTTAACATAATAATGGCTCTGGCTGAGAA
AATTAAACCAGGCCTACACTCTTTTATCTTTGGAAGACCTTTCTACACTAGTGTGCAAGAACGA
GATGTTCTAATGACTTTTCACCACCACCACCACCACTACCCCTACGACGTGCCCGACTACGCCT
AAACAACTTTGTATAATAAAGTTGTAgccttgataacttcgtataatgtatgctatacgaagtt
atccgaatcgcaataacttcgtataaagtatcctatacgaagttatcgaaatcaacctctggat
tacaaaatttgtgaaagattgactggtattcttaactatgttgctccttttacgctatgtggat
acgctgctttaatgcctttgtatcatgctattgcttcccgtatggctttcattttctcctcctt
gtataaatcctggttgctgtctctttatgaggagttgtggcccgttgtcaggcaacgtggcgtg
gtgtgcactgtgtttgctgacgcaacccccactggttggggcattgccaccacctgtcagctcc
tttccgggactttcgctttccccctccctattgccacggcggaactcatcgccgcctgccttgc
ccgctgctggacaggggctcggctgttgggcactgacaattccgtggtgttgtcggggaaatca
tcgtcctttccttggctgctcgcctgtgttgccacctggattctgcgcgggacgtccttctgct
acgtcccttcggccctcaatccagcggaccttccttcccgcggcctgctgccggctctgcggcc
tcttccgcgtcttcgccttcgccctcagacgagtcggatctccctttgggccgcctccccgcct
gctgtgccttctagttgccagccatctgttgtttgcccctcccccgtgccttccttgaccctgg
aaggtgccactcccactgtcctttcctaataaaatgaggaaattgcatcgcattgtctgagtag
gtgtcattctattctggggggtggggtggggcaggacagcaagggggaggattgggaagacaat
agcaggcatgctggggaAACCCAGCTTTcttgtacaaagtggttgatctagagggcccgcggtt
cgaaggtaagcctatccctaaccctctcctcggtctcgattctacgcgtaccggttagtaatga
gtttaaacgggggaggctaactgaaacacggaaggagacaataccggaaggaacccgcgctatg
acggcaataaaaagacagaataaaacgcacgggtgttgggtcgtttgttcataaacgcggggtt
cggtcccagggctggcactctgtcgataccccaccgagaccccattggggccaatacgcccgcg
tttcttccttttccccaccccaccccccaagttcgggtgaaggcccagggctcgcagccaacgt
cggggcggcaggccctgccatagcagatctgcgcagctggggctctagggggtatccccacgcg
ccctgtagcggcgcattaagcgcggcgggtgtggtggttacgcgcagcgtgaccgctacacttg
ccagcgccctagcgcccgctcctttcgctttcttcccttcctttctcgccacgttcgccggctt
tccccgtcaagctctaaatcgggggctccctttagggttccgatttagtgctttacggcacctc
gaccccaaaaaacttgattagggtgatggttcacgtagtgggccatcgccctgatagacggttt
ttcgccctttgacgttggagtccacgttctttaatagtggactcttgttccaaactggaacaac
actcaaccctatctcggtctattcttttgatttataagggattttgccgatttcggcctattgg
ttaaaaaatgagctgatttaacaaaaatttaacgcgaattaattctgtggaatgtgtgtcagtt
agggtgtggaaagtccccaggctccccagcaggcagaagtatgcaaagcatgcatctcaattag
tcagcaaccaggtgtggaaagtccccaggctccccagcaggcagaagtatgcaaagcatgcatc
tcaattagtcagcaaccatagtcccgcccctaactccgcccatcccgcccctaactccgcccag
ttccgcccattctccgccccatggctgactaattttttttatttatgcagaggccgaggccgcc
tctgcctctgagctattccagaagtagtgaggaggcttttttggaggcctaggcttttgcaaaa
agctcccgggagcttgtatatccattttcggatctgatcagcacgtgttgacaattaatcatcg
gcatagtatatcggcatagtataatacgacaaggtgaggaactaaaccatggccaagcctttgt
ctcaagaagaatccaccctcattgaaagagcaacggctacaatcaacagcatccccatctctga
agactacagcgtcgccagcgcagctctctctagcgacggccgcatcttcactggtgtcaatgta
tatcattttactgggggaccttgtgcagaactcgtggtgctgggcactgctgctgctgcggcag
ctggcaacctgacttgtatcgtcgcgatcggaaatgagaacaggggcatcttgagcccctgcgg
acggtgccgacaggtgcttctcgatctgcatcctgggatcaaagccatagtgaaggacagtgat
ggacagccgacggcagttgggattcgtgaattgctgccctctggttatgtgtgggagggctaag
cacttcgtggccgaggagcaggactgacacgtgctacgagatttcgattccaccgccgccttct
atgaaaggttgggcttcggaatcgttttccgggacgccggctggatgatcctccagcgcgggga
tctcatgctggagttcttcgcccaccccaacttgtttattgcagcttataatggttacaaataa
agcaatagcatcacaaatttcacaaataaagcatttttttcactgcattctagttgtggtttgt
ccaaactcatcaatgtatcttatcatgtctgtataccgtcgacctctagctagagcttggcgta
atcatggtcatagctgtttcctgtgtgaaattgttatccgctcacaattccacacaacatacga
gccggaagcataaagtgtaaagcctggggtgcctaatgagtgagctaactcacattaattgcgt
tgcgctcactgcccgctttccagtcgggaaacctgtcgtgccagctgcattaatgaatcggcca
acgcgcggggagaggcggtttgcgtattgggcgctcttccgcttcctcgctcactgactcgctg
cgctcggtcgttcggctgcggcgagcggtatcagctcactcaaaggcggtaatacggttatcca
cagaatcaggggataacgcaggaaagaacatgtgagcaaaaggccagcaaaaggccaggaaccg
taaaaaggccgcgttgctggcgtttttccataggctccgcccccctgacgagcatcacaaaaat
cgacgctcaagtcagaggtggcgaaacccgacaggactataaagataccaggcgtttccccctg
gaagctccctcgtgcgctctcctgttccgaccctgccgcttaccggatacctgtccgcctttct
cccttcgggaagcgtggcgctttctcatagctcacgctgtaggtatctcagttcggtgtaggtc
gttcgctccaagctgggctgtgtgcacgaaccccccgttcagcccgaccgctgcgccttatccg
gtaactatcgtcttgagtccaacccggtaagacacgacttatcgccactggcagcagccactgg
taacaggattagcagagcgaggtatgtaggcggtgctacagagttcttgaagtggtggcctaac
tacggctacactagaagaacagtatttggtatctgcgctctgctgaagccagttaccttcggaa
aaagagttggtagctcttgatccggcaaacaaaccaccgctggtagcggtggtttttttgtttg
caagcagcagattacgcgcagaaaaaaaggatctcaagaagatcctttgatcttttctacgggg
tctgacgctcagtggaacgaaaactcacgttaagggattttggtcatgagattatcaaaaagga
tcttcacctagatccttttaaattaaaaatgaagttttaaatcaatctaaagtatatatgagta
aacttggtctgacagttaccaatgcttaatcagtgaggcacctatctcagcgatctgtctattt
cgttcatccatagttgcctgactccccgtcgtgtagataactacgatacgggagggcttaccat
ctggccccagtgctgcaatgataccgcgagacccacgctcaccggctccagatttatcagcaat
aaaccagccagccggaagggccgagcgcagaagtggtcctgcaactttatccgcctccatccag
tctattaattgttgccgggaagctagagtaagtagttcgccagttaatagtttgcgcaacgttg
ttgccattgctacaggcatcgtggtgtcacgctcgtcgtttggtatggcttcattcagctccgg
ttcccaacgatcaaggcgagttacatgatcccccatgttgtgcaaaaaagcggttagctccttc
ggtcctccgatcgttgtcagaagtaagttggccgcagtgttatcactcatggttatggcagcac
tgcataattctcttactgtcatgccatccgtaagatgcttttctgtgactggtgagtactcaac
caagtcattctgagaatagtgtatgcggcgaccgagttgctcttgcccggcgtcaatacgggat
aataccgcgccacatagcagaactttaaaagtgctcatcattggaaaacgttcttcggggcgaa
aactctcaaggatcttaccgctgttgagatccagttcgatgtaacccactcgtgcacccaactg
atcttcagcatcttttactttcaccagcgtttctgggtgagcaaaaacaggaaggcaaaatgcc
gcaaaaaagggaataagggcgacacggaaatgttgaatactcatactcttcctttttcaatatt
attgaagcatttatcagggttattgtctcatgagcggatacatatttgaatgtatttagaaaaa
taaacaaataggggttccgcgcacatttccccgaaaagtgccacctgacgtcgacggatcggga
gatctcccgatcccctatggtgcactctcagtacaatctgctctgatgccgcatagttaagcca
gtatctgctccctgcttgtgtgttggaggtcgctgagt

According to some embodiments, p111_EXPR-pcDNA-CBA-C9orf72-AI-loxp-WPRE-pA_4-018_FP-CBA (1153 bp) comprises SEQ ID NO: 60, shown below.

NNNNNNNNNNNNNNNNNNNNNNTGTTCNTGCCTTCTTCTTTTTCCTACAG
CTCCTGGGCAACGCCACCATGGACAACTTTGTATACAAAAGTTGTAGCCA
CCATGTCGACTCTTTGCCCACCGCCATCTCCAGCTGTTGCCAAGACAGAG
ATTGCTTTAAGTGGCAAATCACCTTTATTAGCAGCTACTTTTGCTTACTG
GGACAATATTCTTGGTCCTAGAGTAAGGCACATTTGGGCTCCAAAGACAG
AACAGGTACTTCTCAGTGATGGAGAAATAACTTTTCTTGCCAACCACACT
CTAAATGGAGAAATCCTTCGAAATGCAGAGAGTGGTGCTATAGATGTAAA
GTTTTTTGTCTTGTCTGAAAAGGGAGTGATTATTGTTTCATTAATCTTTG
ATGGAAACTGGAATGGGGATCGCAGCACATATGGACTATCAATTATACTT
CCACAGACAGAACTTAGTTTCTACCTCCCACTTCATAGAGTGTGTGTTGA
TAGATTAACACATATAATCCGGAAAGGAAGAATATGGATGCATAAGGAAA
GACAAGAAAATGTCCAGAAGATTATCTTAGAAGGCACAGAGAGAATGGAA
GATCAGGGTCAGAGTATTATTCCAATGCTTACTGGAGAAGTGATTCCTGT
AATGGAACTGCTTTCATCTATGAAATCACACAGTGTTCCTGAAGAAATAG
ATATAGCTGATACAGTACTCAATGATGATGATATTGGTGACAGCTGTCAT
GAAGGCTTTCTTCTCGTAAGTCGACTCGTTGGATCCCCACTACAGCCGAT
ACTCAAGCTTGACGAATTCGACCACCACCACCACCACCACGAGCAGAAGC
TGATCTCCGAGGAGGANCTGTAACACCCAACTTTTCTATACAAAGTTGTA
GTATCCANGGTAGTGGNCTANTGTGACGCTGCTGACCCCTTTCTTTCCCT
TCTGCAGAATGCCATCAGCTCACACTTGCAAACCTGTGGCTNGTTCCGTT
GTAGTNNNAGCANTGCANANAANTAAATAAGATAGNCNNANCNTNNTGCC
TTTTTCTGACTCAGCANAANANAAAATGCTCCANGNNNNNNTGNAGCNNN
ANCATTCNTTTAAAATNNTGAGNNNNGGCNNNTTTNGNNNNNNNANGNNN
NGN

According to some embodiments, p111_EXPR-pcDNA-CBA-C9orf72-AI-loxp-WPRE-pA_4-RP-WPRE-01 (645 bp) comprises SEQ ID NO: 61, shown below.

NNNNNNNNNNNNNNNNNTNNNNCAGCGTATCCACATAGCGTAAAAGGAGC
AACATAGTTAAGAATACCAGTCAATCTTTCACAAATTTTGTAATCCAGAG
GTTGATTTCGATAACTTCGTATAGGATACTTTATACGAAGTTATTGCGAT
TCGGATAACTTCGTATAGCATACATTATACGAAGTTATCAAGGCTACAAC
TTTATTATACAAAGTTGTTTAGGCGTAGTCGGGCACGTCGTAGGGGTAGT
GGTGGTGGTGGTGGTGAAAAGTCATTATAACATCTCGTTCTTGCACACTA
GTGTAGAAAGGTCTTCCAAAGATAAAAGAGTGTAGGCCTGGTTTAATTTT
CTCAGCCAGAGCCATTATTATGTTAAGATCGCCCTCTGCTGTTAAATCAA
GGTCTATCTTCAGGTTCCGAAGAGATTTAAAGGGCTTTTTTCCCTTCTGC
GTATCGTCTTCTATATATTTTATTAGTGTCAAGGCTTTTCTGTGAAGGAC
AAGTAGAAACTGTGCAAGGAAAGTACTTCTGAGAGATAAGCCAGGTTTCA
GCTGAAAGACCTGATCCAGGAAGGCTTTCACTAGAGTGTCTCTGTGTAAA
ACATCTTGAAAAATATTCCAATCAGGAGTATAGCTTTCGTCAGTN

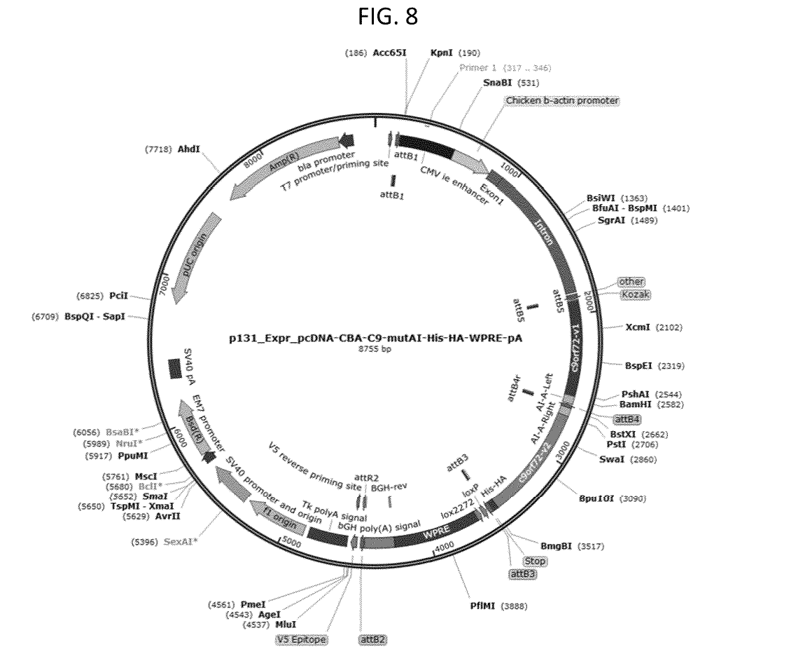

(4) p131_Expr_pcDNA-CBA-C9-mutAI-His-HA-WPRE-pA. This construct comprises CBA promoter, polyA signal, Ampicillin resistance gene. This construct carry a C9orf72 sequence designed to express long C9orf72 protein isoform tagged with His and HA, a short C90rf72 protein isoform tagged with no tag. The vector map is shown in FIG. 8. According to some embodiments, the nucleic acid sequence of p131_Expr_pcDNA-CBA-C9-mutAI-His-HA-WPRE-pA comprises SEQ ID NO: 62. According to some embodiments, the nucleic acid sequence of p131_Expr_pcDNA-CBA-C9-mutAI-His-HA-WPRE-pA is at least 85%, at least 90%, at least 95%, at least 96%, at least 97%, at least 98%, at least 99% identical to SEQ ID NO: 62, shown below.

agtgcgcgagcaaaatttaagctacaacaaggcaaggcttgaccgacaattctctggctaacta
gagaacccactgcttactggcttatcgaaattaatacgactcactatagggagacccaagctgg
ctagttaagctatcaacaagtttGTACAAAAAAGCAGGCTTActcagatctgaattcggtacct
agttattaatagtaatcaattacggggtcattagttcatagcccatatatggagttccgcgtta
cataacttacggtaaatggcccgcctggctgaccgcccaacgacccccgcccattgacgtcaat
aatgacgtatgttcccatagtaacgccaatagggactttccattgacgtcaatgggtggagtat
ttacggtaaactgcccacttggcagtacatcaagtgtatcatatgccaagtacgccccctattg
acgtcaatgacggtaaatggcccgcctggcattatgcccagtacatgaccttatgggactttcc
tacttggcagtacatctacgtattagtcatcgctattaccatggtcgaggtgagccccacgttc
tgcttcactctccccatctcccccccctccccacccccaattttgtatttatttattttttaat
tattttgtgcagcgatgggggcggggggggggggggggcgcgcgccaggcggggcggggcgggg
cgaggggcggggcggggcgaggcggagaggtgcggcggcagccaatcagagcggcgcgctccga
aagtttccttttatggcgaggcggcggcggcggcggccctataaaaagcgaagcgcgcggcggg
cgggagtcgctgcgcgctgccttcgccccgtgccccgctccgccgccgcctcgcgccgcccgcc
ccggctctgactgaccgcgttactcccacaggtgagcgggcgggacggcccttctcctccgggc
tgtaattagcgcttggtttaatgacggcttgtttcttttctgtggctgcgtgaaagccttgagg
ggctccgggagggccctttgtgcggggggagcggctcggggggtgcgtgcgtgtgtgtgtgcgt
ggggagcgccgcgtgcggctccgcgctgcccggcggctgtgagcgctgcgggcgcggcgcgggg
ctttgtgcgctccgcagtgtgcgcgaggggagcgcggccgggggcggtgccccgcggtgcgggg
ggggctgcgaggggaacaaaggctgcgtgcggggtgtgtgcgtgggggggtgagcagggggtgt
gggcgcgtcggtcgggctgcaaccccccctgcacccccctccccgagttgctgagcacggcccg
gcttcgggtgcggggctccgtacggggcgtggcgcggggctcgccgtgccgggcggggggtggc
ggcaggtgggggtgccgggcggggcggggccgcctcgggccggggagggctcgggggaggggcg
cggcggcccccggagcgccggcggctgtcgaggcgcggcgagccgcagccattgccttttatgg
taatcgtgcgagagggcgcagggacttcctttgtcccaaatctgtgcggagccgaaatctggga
ggcgccgccgcaccccctctagcgggcgcggggcgaagcggtgcggcgccggcaggaaggaaat
gggcggggagggccttcgtgcgtcgccgcgccgccgtccccttctccctctccagcctcggggc
tgtccgcggggggacggctgccttcgggggggacggggcagggcggggttcggcttctggcgtg
tgaccggcggctctagagcctctgctaaccatgttcatgccttcttctttttcctacagctcct
gggcaacgccaccatggACAACTTTGTATACAAAAGTTGTAgccaccATGTCGACTCTTTGCCC
ACCGCCATCTCCAGCTGTTGCCAAGACAGAGATTGCTTTAAGTGGCAAATCACCTTTATTAGCA
GCTACTTTTGCTTACTGGGACAATATTCTTGGTCCTAGAGTAAGGCACATTTGGGCTCCAAAGA
CAGAACAGGTACTTCTCAGTGATGGAGAAATAACTTTTCTTGCCAACCACACTCTAAATGGAGA
AATCCTTCGAAATGCAGAGAGTGGTGCTATAGATGTAAAGTTTTTTGTCTTGTCTGAAAAGGGA
GTGATTATTGTTTCATTAATCTTTGATGGAAACTGGAATGGGGATCGCAGCACATATGGACTAT
CAATTATACTTCCACAGACAGAACTTAGTTTCTACCTCCCACTTCATAGAGTGTGTGTTGATAG
ATTAACACATATAATCCGGAAAGGAAGAATATGGATGCATAAGGAAAGACAAGAAAATGTCCAG
AAGATTATCTTAGAAGGCACAGAGAGAATGGAAGATCAGGGTCAGAGTATTATTCCAATGCTTA
CTGGAGAAGTGATTCCTGTAATGGAACTGCTTTCATCTATGAAATCACACAGTGTTCCTGAAGA
AATAGATATAGCTGATACAGTACTCAATGATGATGATATTGGTGACAGCTGTCATGAAGGCTTT
CTTCTCgtaagtTgactcgttggatccccactacagccgatactcaagcttgacgaattcgacC
ACCCAACTTTTCTATACAAAGTTGTAgtatccaaggtagtggactagtgtgacgctgctgaccc
ctttctttcccttctgcagAATGCCATCAGCTCACACTTGCAAACCTGTGGCTGTTCCGTTGTA
GTAGGTAGCAGTGCAGAGAAAGTAAATAAGATAGTCAGAACATTATGCCTTTTTCTGACTCCAG
CAGAGAGAAAATGCTCCAGGTTATGTGAAGCAGAATCATCATTTAAATATGAGTCAGGGCTCTT
TGTACAAGGCCTGCTAAAGGATTCAACTGGAAGCTTTGTGCTGCCTTTCCGGCAAGTCATGTAT
GCTCCATATCCCACCACACACATAGATGTGGATGTCAATACTGTGAAGCAGATGCCACCCTGTC
ATGAACATATTTATAATCAGCGTAGATACATGAGATCCGAGCTGACAGCCTTCTGGAGAGCCAC
TTCAGAAGAAGACATGGCTCAGGATACGATCATCTACACTGACGAAAGCTTTACTCCTGATTTG
AATATTTTTCAAGATGTCTTACACAGAGACACTCTAGTGAAAGCCTTCCTGGATCAGGTCTTTC
AGCTGAAACCTGGCTTATCTCTCAGAAGTACTTTCCTTGCACAGTTTCTACTTGTCCTTCACAG
AAAAGCCTTGACACTAATAAAATATATAGAAGACGATACGCAGAAGGGAAAAAAGCCCTTTAAA
TCTCTTCGGAACCTGAAGATAGACCTTGATTTAACAGCAGAGGGCGATCTTAACATAATAATGG
CTCTGGCTGAGAAAATTAAACCAGGCCTACACTCTTTTATCTTTGGAAGACCTTTCTACACTAG
TGTGCAAGAACGAGATGTTCTAATGACTTTTCACCACCACCACCACCACTACCCCTACGACGTG
CCCGACTACGCCTAAACAACTTTGTATAATAAAGTTGTAgccttgataacttcgtataatgtat
gctatacgaagttatccgaatcgcaataacttcgtataaagtatcctatacgaagttatcgaaa
tcaacctctggattacaaaatttgtgaaagattgactggtattcttaactatgttgctcctttt
acgctatgtggatacgctgctttaatgcctttgtatcatgctattgcttcccgtatggctttca
ttttctcctccttgtataaatcctggttgctgtctctttatgaggagttgtggcccgttgtcag
gcaacgtggcgtggtgtgcactgtgtttgctgacgcaacccccactggttggggcattgccacc
acctgtcagctcctttccgggactttcgctttccccctccctattgccacggcggaactcatcg
ccgcctgccttgcccgctgctggacaggggctcggctgttgggcactgacaattccgtggtgtt
gtcggggaaatcatcgtcctttccttggctgctcgcctgtgttgccacctggattctgcgcggg
acgtccttctgctacgtcccttcggccctcaatccagcggaccttccttcccgcggcctgctgc
cggctctgcggcctcttccgcgtcttcgccttcgccctcagacgagtcggatctccctttgggc
cgcctccccgcctgctgtgccttctagttgccagccatctgttgtttgcccctcccccgtgcct
tccttgaccctggaaggtgccactcccactgtcctttcctaataaaatgaggaaattgcatcgc
attgtctgagtaggtgtcattctattctggggggtggggtggggcaggacagcaagggggagga
ttgggaagacaatagcaggcatgctggggaAACCCAGCTTTcttgtacaaagtggttgatctag
agggcccgcggttcgaaggtaagcctatccctaaccctctcctcggtctcgattctacgcgtac
cggttagtaatgagtttaaacgggggaggctaactgaaacacggaaggagacaataccggaagg
aacccgcgctatgacggcaataaaaagacagaataaaacgcacgggtgttgggtcgtttgttca
taaacgcggggttcggtcccagggctggcactctgtcgataccccaccgagaccccattggggc
caatacgcccgcgtttcttccttttccccaccccaccccccaagttcgggtgaaggcccagggc
tcgcagccaacgtcggggcggcaggccctgccatagcagatctgcgcagctggggctctagggg
gtatccccacgcgccctgtagcggcgcattaagcgcggcgggtgtggtggttacgcgcagcgtg
accgctacacttgccagcgccctagcgcccgctcctttcgctttcttcccttcctttctcgcca
cgttcgccggctttccccgtcaagctctaaatcgggggctccctttagggttccgatttagtgc
tttacggcacctcgaccccaaaaaacttgattagggtgatggttcacgtagtgggccatcgccc
tgatagacggtttttcgccctttgacgttggagtccacgttctttaatagtggactcttgttcc
aaactggaacaacactcaaccctatctcggtctattcttttgatttataagggattttgccgat
ttcggcctattggttaaaaaatgagctgatttaacaaaaatttaacgcgaattaattctgtgga
atgtgtgtcagttagggtgtggaaagtccccaggctccccagcaggcagaagtatgcaaagcat
gcatctcaattagtcagcaaccaggtgtggaaagtccccaggctccccagcaggcagaagtatg
caaagcatgcatctcaattagtcagcaaccatagtcccgcccctaactccgcccatcccgcccc
taactccgcccagttccgcccattctccgccccatggctgactaattttttttatttatgcaga
ggccgaggccgcctctgcctctgagctattccagaagtagtgaggaggcttttttggaggccta
ggcttttgcaaaaagctcccgggagcttgtatatccattttcggatctgatcagcacgtgttga
caattaatcatcggcatagtatatcggcatagtataatacgacaaggtgaggaactaaaccatg
gccaagcctttgtctcaagaagaatccaccctcattgaaagagcaacggctacaatcaacagca
tccccatctctgaagactacagcgtcgccagcgcagctctctctagcgacggccgcatcttcac
tggtgtcaatgtatatcattttactgggggaccttgtgcagaactcgtggtgctgggcactgct
gctgctgcggcagctggcaacctgacttgtatcgtcgcgatcggaaatgagaacaggggcatct
tgagcccctgcggacggtgccgacaggtgcttctcgatctgcatcctgggatcaaagccatagt
gaaggacagtgatggacagccgacggcagttgggattcgtgaattgctgccctctggttatgtg
tgggagggctaagcacttcgtggccgaggagcaggactgacacgtgctacgagatttcgattcc
accgccgccttctatgaaaggttgggcttcggaatcgttttccgggacgccggctggatgatcc
tccagcgcggggatctcatgctggagttcttcgcccaccccaacttgtttattgcagcttataa
tggttacaaataaagcaatagcatcacaaatttcacaaataaagcatttttttcactgcattct
agttgtggtttgtccaaactcatcaatgtatcttatcatgtctgtataccgtcgacctctagct
agagcttggcgtaatcatggtcatagctgtttcctgtgtgaaattgttatccgctcacaattcc
acacaacatacgagccggaagcataaagtgtaaagcctggggtgcctaatgagtgagctaactc
acattaattgcgttgcgctcactgcccgctttccagtcgggaaacctgtcgtgccagctgcatt
aatgaatcggccaacgcgcggggagaggcggtttgcgtattgggcgctcttccgcttcctcgct
cactgactcgctgcgctcggtcgttcggctgcggcgagcggtatcagctcactcaaaggcggta
atacggttatccacagaatcaggggataacgcaggaaagaacatgtgagcaaaaggccagcaaa
aggccaggaaccgtaaaaaggccgcgttgctggcgtttttccataggctccgcccccctgacga
gcatcacaaaaatcgacgctcaagtcagaggtggcgaaacccgacaggactataaagataccag
gcgtttccccctggaagctccctcgtgcgctctcctgttccgaccctgccgcttaccggatacc
tgtccgcctttctcccttcgggaagcgtggcgctttctcatagctcacgctgtaggtatctcag
ttcggtgtaggtcgttcgctccaagctgggctgtgtgcacgaaccccccgttcagcccgaccgc
tgcgccttatccggtaactatcgtcttgagtccaacccggtaagacacgacttatcgccactgg
cagcagccactggtaacaggattagcagagcgaggtatgtaggcggtgctacagagttcttgaa
gtggtggcctaactacggctacactagaagaacagtatttggtatctgcgctctgctgaagcca
gttaccttcggaaaaagagttggtagctcttgatccggcaaacaaaccaccgctggtagcggtg
gtttttttgtttgcaagcagcagattacgcgcagaaaaaaaggatctcaagaagatcctttgat
cttttctacggggtctgacgctcagtggaacgaaaactcacgttaagggattttggtcatgaga
ttatcaaaaaggatcttcacctagatccttttaaattaaaaatgaagttttaaatcaatctaaa
gtatatatgagtaaacttggtctgacagttaccaatgcttaatcagtgaggcacctatctcagc
gatctgtctatttcgttcatccatagttgcctgactccccgtcgtgtagataactacgatacgg
gagggcttaccatctggccccagtgctgcaatgataccgcgagacccacgctcaccggctccag
atttatcagcaataaaccagccagccggaagggccgagcgcagaagtggtcctgcaactttatc
cgcctccatccagtctattaattgttgccgggaagctagagtaagtagttcgccagttaatagt
ttgcgcaacgttgttgccattgctacaggcatcgtggtgtcacgctcgtcgtttggtatggctt
cattcagctccggttcccaacgatcaaggcgagttacatgatcccccatgttgtgcaaaaaagc
ggttagctccttcggtcctccgatcgttgtcagaagtaagttggccgcagtgttatcactcatg
gttatggcagcactgcataattctcttactgtcatgccatccgtaagatgcttttctgtgactg
gtgagtactcaaccaagtcattctgagaatagtgtatgcggcgaccgagttgctcttgcccggc
gtcaatacgggataataccgcgccacatagcagaactttaaaagtgctcatcattggaaaacgt
tcttcggggcgaaaactctcaaggatcttaccgctgttgagatccagttcgatgtaacccactc
gtgcacccaactgatcttcagcatcttttactttcaccagcgtttctgggtgagcaaaaacagg
aaggcaaaatgccgcaaaaaagggaataagggcgacacggaaatgttgaatactcatactcttc
ctttttcaatattattgaagcatttatcagggttattgtctcatgagcggatacatatttgaat
gtatttagaaaaataaacaaataggggttccgcgcacatttccccgaaaagtgccacctgacgt
cgacggatcgggagatctcccgatcccctatggtgcactctcagtacaatctgctctgatgccg
catagttaagccagtatctgctccctgcttgtgtgttggaggtcgctgagt

According to some embodiments, p131_Expr_pcDNA-CBA-C9-mutAI-His-HA-WPRE-pA_6-FP-CBA (1079 bp) comprises SEQ ID NO: 63, shown below.

NNNNNNNNNNNNNNNNNNCNNNNTGTTCNTGCCTTCTTCTTTTTCCTACA
GCTCCTGGGCAACGCCACCATGGACAACTTTGTATACAAAAGTTGTAGCC
ACCATGTCGACTCTTTGCCCACCGCCATCTCCAGCTGTTGCCAAGACAGA
GATTGCTTTAAGTGGCAAATCACCTTTATTAGCAGCTACTTTTGCTTACT
GGGACAATATTCTTGGTCCTAGAGTAAGGCACATTTGGGCTCCAAAGACA
GAACAGGTACTTCTCAGTGATGGAGAAATAACTTTTCTTGCCAACCACAC
TCTAAATGGAGAAATCCTTCGAAATGCAGAGAGTGGTGCTATAGATGTAA
AGTTTTTTGTCTTGTCTGAAAAGGGAGTGATTATTGTTTCATTAATCTTT
GATGGAAACTGGAATGGGGATCGCAGCACATATGGACTATCAATTATACT
TCCACAGACAGAACTTAGTTTCTACCTCCCACTTCATAGAGTGTGTGTTG
ATAGATTAACACATATAATCCGGAAAGGAAGAATATGGATGCATAAGGAA
AGACAAGAAAATGTCCAGAAGATTATCTTAGAAGGCACAGAGAGAATGGA
AGATCAGGGTCAGAGTATTATTCCAATGCTTACTGGAGAAGTGATTCCTG
TAATGGAACTGCTTTCATCTATGAAATCACACAGTGTTCCTGAAGAAATA
GATATAGCTGATACAGTACTCAATGATGATGATATTGGTGACAGCTGTCA
TGAAGGCTTTCTTCTCGTAAGTTGACTCGTTGGATCCCCACTACAGCCGA
TACTCAAGCTTNGACGAATTCGACCACCCAACTTTTCTATACAAAGTTGT
AGTATCCNAAGGTAGTGGACTAGTGTGACGCTGCTGACCCCTTTCTTTCC
CTTCNTGCAGAATGCCATCAGCTCACACTTGCAAACCTGTGGCTGNTCCG
TTGTAGTANNAGCAGTGCAGANAANNNAATANNANAGTCNNAACATTATG
CCTTTTCTGACTCCAGCANAANANAAAATGCTCCAGGTTATGTGAAGCNA
ANTCATCATTTAAATATGAGTNNNNNNNN

According to some embodiments, p131_Expr_pcDNA-CBA-C9-mutAI-His-HA-WPRE-pA_6-RP-WPRE-01 (1058 bp) comprises SEQ ID NO: 64, shown below.

NNNNNNNNNNNNNGNNTNNNNNNCAGCGTATCCNCATAGCGTAAAAGGAG
CAACATAGTTAAGAATACCAGTCAATCTTTCANAAATTTTGTAATCCAGA
GGTTGATTTCGATAACTTCGTATAGGATACTTTATACGAAGTTATTGCGA
TTCGGATAACTTCGTATAGCATACATTATACGAAGTTATCAAGGCTACAA
CTTTATTATACAAAGTTGTTTAGGCGTAGTCGGGCACGTCGTAGGGGTAG
TGGTGGTGGTGGTGGTGAAAAGTCATTAGAACATCTCGTTCTTGCACACT
AGTGTAGAAAGGTCTTCCAAAGATAAAAGAGTGTAGGCCTGGTTTAATTT
TCTCAGCCAGAGCCATTATTATGTTAAGATCGCCCTCTGCTGTTAAATCA
AGGTCTATCTTCAGGTTCCGAAGAGATTTAAAGGGCTTTTTTCCCTTCTG
CGTATCGTCTTCTATATATTTTATTAGTGTCAAGGCTTTTCTGTGAAGGA
CAAGTAGAAACTGTGCAAGGAAAGTACTTCTGAGAGATAAGCCAGGTTTC
AGCTGAAAGACCTGATCCAGGAAGGCTTTCACTAGAGTGTCTCTGTGTAA
GACATCTTGAAAAATATTCAAATCAGGAGTAAAGCTTTCGTCAGTGTAGA
TGATCGTATCCTGAGCCATGTCTTCTTCTGAAGTGGCTCTCCAGAAGGCT
GTCAGCTCGGATCTCATGTATCTACGCTGATTATAAATATGTTCATGACA
GGGTGGCATCTGCTTCACAGTATTGACATCCACATCTATGTGTGTGGNGG
GATATGGAGCATACATGACTTTGCCGGAAAGGCAGCACAAAGCTTCCAGT
TGAATCCTTTTAGCNNCCTTGTACAAAGAGCCCTGACTCATATTTTAAAT
GATGATTCTGCTTCACATAACCTGGAGCATTTTCTCTCNNGCTGGGAGTC
AGAAAAGGGCNTAATGTTCTNGACTNATCTTANTTACTTTCTCTGCACCN
GCCTACCTACTACANNGNANCANNCCACAGGNTTTGCAAGTGGTGANCNN
ATGGCNAT

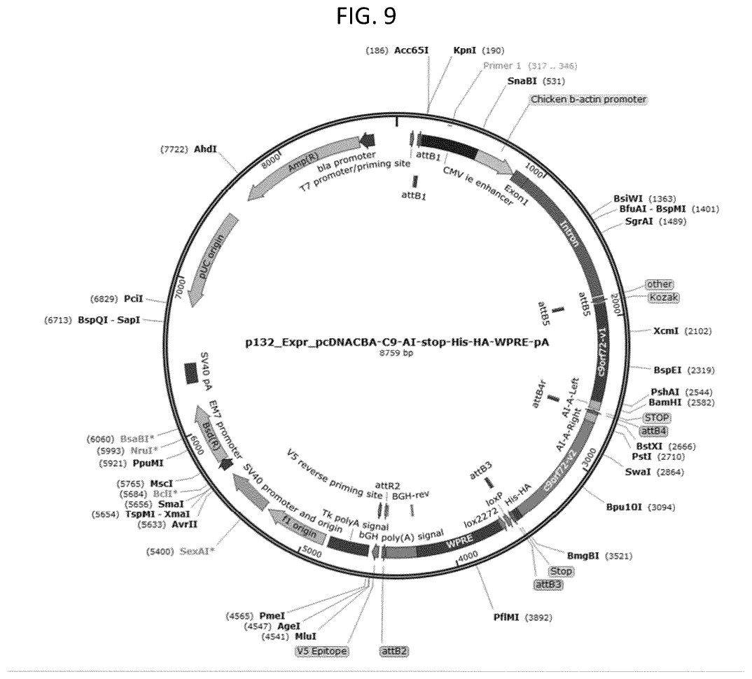

(5) p132_Expr_pcDNACBA-C9-AI-stop-His-HA-WPRE-pA. This construct comprises a C9orf72 sequence designed to express long C9orf72 protein isoform tagged with His and HA, a short C9Orf72 protein isoform tagged with no tag. The vector map is shown in FIG. 9.

According to some embodiments, the nucleic acid sequence of p132_Expr_pcDNACBA-C9-AI-stop-His-HA-WPRE-pA comprises SEQ ID NO: 65. According to some embodiments, the nucleic acid sequence of p132_Expr_pcDNACBA-C9-AI-stop-His-HA-WPRE-pA is at least 85%, at least 90%, at least 95%, at least 96%, at least 97%, at least 98%, at least 99% identical to SEQ ID NO: 65, shown below.

agtgcgcgagcaaaatttaagctacaacaaggcaaggcttgaccgacaattctctggctaacta
gagaacccactgcttactggcttatcgaaattaatacgactcactatagggagacccaagctgg
ctagttaagctatcaacaagtttGTACAAAAAAGCAGGCTTActcagatctgaattcggtacct
agttattaatagtaatcaattacggggtcattagttcatagcccatatatggagttccgcgtta
cataacttacggtaaatggcccgcctggctgaccgcccaacgacccccgcccattgacgtcaat
aatgacgtatgttcccatagtaacgccaatagggactttccattgacgtcaatgggtggagtat
ttacggtaaactgcccacttggcagtacatcaagtgtatcatatgccaagtacgccccctattg
acgtcaatgacggtaaatggcccgcctggcattatgcccagtacatgaccttatgggactttcc
tacttggcagtacatctacgtattagtcatcgctattaccatggtcgaggtgagccccacgttc
tgcttcactctccccatctcccccccctccccacccccaattttgtatttatttattttttaat
tattttgtgcagcgatgggggcggggggggggggggggcgcgcgccaggcggggcggggcgggg
cgaggggcggggcggggcgaggcggagaggtgcggcggcagccaatcagagcggcgcgctccga
aagtttccttttatggcgaggcggcggcggcggcggccctataaaaagcgaagcgcgcggcggg
cgggagtcgctgcgcgctgccttcgccccgtgccccgctccgccgccgcctcgcgccgcccgcc
ccggctctgactgaccgcgttactcccacaggtgagcgggcgggacggcccttctcctccgggc
tgtaattagcgcttggtttaatgacggcttgtttcttttctgtggctgcgtgaaagccttgagg
ggctccgggagggccctttgtgcggggggagcggctcggggggtgcgtgcgtgtgtgtgtgcgt
ggggagcgccgcgtgcggctccgcgctgcccggcggctgtgagcgctgcgggcgcggcgcgggg
ctttgtgcgctccgcagtgtgcgcgaggggagcgcggccgggggcggtgccccgcggtgcgggg
ggggctgcgaggggaacaaaggctgcgtgcggggtgtgtgcgtgggggggtgagcagggggtgt
gggcgcgtcggtcgggctgcaaccccccctgcacccccctccccgagttgctgagcacggcccg
gcttcgggtgcggggctccgtacggggcgtggcgcggggctcgccgtgccgggcggggggtggc
ggcaggtgggggtgccgggcggggcggggccgcctcgggccggggagggctcgggggaggggcg
cggcggcccccggagcgccggcggctgtcgaggcgcggcgagccgcagccattgccttttatgg
taatcgtgcgagagggcgcagggacttcctttgtcccaaatctgtgcggagccgaaatctggga
ggcgccgccgcaccccctctagcgggcgcggggcgaagcggtgcggcgccggcaggaaggaaat
gggcggggagggccttcgtgcgtcgccgcgccgccgtccccttctccctctccagcctcggggc
tgtccgcggggggacggctgccttcgggggggacggggcagggcggggttcggcttctggcgtg
tgaccggcggctctagagcctctgctaaccatgttcatgccttcttctttttcctacagctcct
gggcaacgccaccatggACAACTTTGTATACAAAAGTTGTAgccaccATGTCGACTCTTTGCCC
ACCGCCATCTCCAGCTGTTGCCAAGACAGAGATTGCTTTAAGTGGCAAATCACCTTTATTAGCA
GCTACTTTTGCTTACTGGGACAATATTCTTGGTCCTAGAGTAAGGCACATTTGGGCTCCAAAGA
CAGAACAGGTACTTCTCAGTGATGGAGAAATAACTTTTCTTGCCAACCACACTCTAAATGGAGA
AATCCTTCGAAATGCAGAGAGTGGTGCTATAGATGTAAAGTTTTTTGTCITGTCTGAAAAGGGA
GTGATTATTGTTICATTAATCITTGATGGAAACTGGAATGGGGATCGCAGCACATATGGACTAT
CAATTATACTTCCACAGACAGAACTTAGTTTCTACCTCCCACTTCATAGAGTGTGTGTTGATAG
ATTAACACATATAATCCGGAAAGGAAGAATATGGATGCATAAGGAAAGACAAGAAAATGTCCAG
AAGATTATCTTAGAAGGCACAGAGAGAATGGAAGATCAGGGTCAGAGTATTATTCCAATGCTTA
CTGGAGAAGTGATTCCTGTAATGGAACTGCTTTCATCTATGAAATCACACAGTGTTCCTGAAGA
AATAGATATAGCTGATACAGTACTCAATGATGATGATATTGGTGACAGCTGTCATGAAGGCTTT
CTTCTCgtaagtcgactcgttggatccccactacagccgatactcaagcttgacgaattcgacT
GACCACCCAACTTTTCTATACAAAGTTGTAgtatccaaggtagtggactagtgtgacgctgctg
acccctttctttcccttctgcagAATGCCATCAGCTCACACTTGCAAACCTGTGGCTGTTCCGT
TGTAGTAGGTAGCAGTGCAGAGAAAGTAAATAAGATAGTCAGAACATTATGCCTTTTTCTGACT
CCAGCAGAGAGAAAATGCTCCAGGTTATGTGAAGCAGAATCATCATTTAAATATGAGTCAGGGC
TCTTTGTACAAGGCCTGCTAAAGGATTCAACTGGAAGCTTTGTGCTGCCTTTCCGGCAAGTCAT
GTATGCTCCATATCCCACCACACACATAGATGTGGATGTCAATACTGTGAAGCAGATGCCACCC
TGTCATGAACATATTTATAATCAGCGTAGATACATGAGATCCGAGCTGACAGCCTTCTGGAGAG
CCACTTCAGAAGAAGACATGGCTCAGGATACGATCATCTACACTGACGAAAGCTTTACTCCTGA
TTTGAATATTTTTCAAGATGTCTTACACAGAGACACTCTAGTGAAAGCCTTCCTGGATCAGGTC
TTTCAGCTGAAACCTGGCTTATCTCTCAGAAGTACTTTCCTTGCACAGTTTCTACTTGTCCTTC
ACAGAAAAGCCTTGACACTAATAAAATATATAGAAGACGATACGCAGAAGGGAAAAAAGCCCTT
TAAATCTCTTCGGAACCTGAAGATAGACCTTGATTTAACAGCAGAGGGCGATCTTAACATAATA
ATGGCTCTGGCTGAGAAAATTAAACCAGGCCTACACTCTTTTATCTTTGGAAGACCTTTCTACA
CTAGTGTGCAAGAACGAGATGTTCTAATGACTTTTCACCACCACCACCACCACTACCCCTACGA
CGTGCCCGACTACGCCTAAACAACTTTGTATAATAAAGTTGTAgccttgataacttcgtataat
gtatgctatacgaagttatccgaatcgcaataacttcgtataaagtatcctatacgaagttatc
gaaatcaacctctggattacaaaatttgtgaaagattgactggtattcttaactatgttgctcc
ttttacgctatgtggatacgctgctttaatgcctttgtatcatgctattgcttcccgtatggct
ttcattttctcctccttgtataaatcctggttgctgtctctttatgaggagttgtggcccgttg
tcaggcaacgtggcgtggtgtgcactgtgtttgctgacgcaacccccactggttggggcattgc
caccacctgtcagctcctttccgggactttcgctttccccctccctattgccacggcggaactc
atcgccgcctgccttgcccgctgctggacaggggctcggctgttgggcactgacaattccgtgg
tgttgtcggggaaatcatcgtcctttccttggctgctcgcctgtgttgccacctggattctgcg
cgggacgtccttctgctacgtcccttcggccctcaatccagcggaccttccttcccgcggcctg
ctgccggctctgcggcctcttccgcgtcttcgccttcgccctcagacgagtcggatctcccttt
gggccgcctccccgcctgctgtgccttctagttgccagccatctgttgtttgcccctcccccgt
gccttccttgaccctggaaggtgccactcccactgtcctttcctaataaaatgaggaaattgca
tcgcattgtctgagtaggtgtcattctattctggggggtggggtggggcaggacagcaaggggg
aggattgggaagacaatagcaggcatgctggggaAACCCAGCTTTcttgtacaaagtggttgat
ctagagggcccgcggttcgaaggtaagcctatccctaaccctctcctcggtctcgattctacgc
gtaccggttagtaatgagtttaaacgggggaggctaactgaaacacggaaggagacaataccgg
aaggaacccgcgctatgacggcaataaaaagacagaataaaacgcacgggtgttgggtcgtttg
ttcataaacgcggggttcggtcccagggctggcactctgtcgataccccaccgagaccccattg
gggccaatacgcccgcgtttcttccttttccccaccccaccccccaagttcgggtgaaggccca
gggctcgcagccaacgtcggggcggcaggccctgccatagcagatctgcgcagctggggctcta
gggggtatccccacgcgccctgtagcggcgcattaagcgcggcgggtgtggtggttacgcgcag
cgtgaccgctacacttgccagcgccctagcgcccgctcctttcgctttcttcccttcctttctc
gccacgttcgccggctttccccgtcaagctctaaatcgggggctccctttagggttccgattta
gtgctttacggcacctcgaccccaaaaaacttgattagggtgatggttcacgtagtgggccatc
gccctgatagacggtttttcgccctttgacgttggagtccacgttctttaatagtggactcttg
ttccaaactggaacaacactcaaccctatctcggtctattcttttgatttataagggattttgc
cgatttcggcctattggttaaaaaatgagctgatttaacaaaaatttaacgcgaattaattctg
tggaatgtgtgtcagttagggtgtggaaagtccccaggctccccagcaggcagaagtatgcaaa
gcatgcatctcaattagtcagcaaccaggtgtggaaagtccccaggctccccagcaggcagaag
tatgcaaagcatgcatctcaattagtcagcaaccatagtcccgcccctaactccgcccatcccg
cccctaactccgcccagttccgcccattctccgccccatggctgactaattttttttatttatg
cagaggccgaggccgcctctgcctctgagctattccagaagtagtgaggaggcttttttggagg
cctaggcttttgcaaaaagctcccgggagcttgtatatccattttcggatctgatcagcacgtg
ttgacaattaatcatcggcatagtatatcggcatagtataatacgacaaggtgaggaactaaac
catggccaagcctttgtctcaagaagaatccaccctcattgaaagagcaacggctacaatcaac
agcatccccatctctgaagactacagcgtcgccagcgcagctctctctagcgacggccgcatct
tcactggtgtcaatgtatatcattttactgggggaccttgtgcagaactcgtggtgctgggcac
tgctgctgctgcggcagctggcaacctgacttgtatcgtcgcgatcggaaatgagaacaggggc
atcttgagcccctgcggacggtgccgacaggtgcttctcgatctgcatcctgggatcaaagcca
tagtgaaggacagtgatggacagccgacggcagttgggattcgtgaattgctgccctctggtta
tgtgtgggagggctaagcacttcgtggccgaggagcaggactgacacgtgctacgagatttcga
ttccaccgccgccttctatgaaaggttgggcttcggaatcgttttccgggacgccggctggatg
atcctccagcgcggggatctcatgctggagttcttcgcccaccccaacttgtttattgcagctt
ataatggttacaaataaagcaatagcatcacaaatttcacaaataaagcatttttttcactgca
ttctagttgtggtttgtccaaactcatcaatgtatcttatcatgtctgtataccgtcgacctct
agctagagcttggcgtaatcatggtcatagctgtttcctgtgtgaaattgttatccgctcacaa
ttccacacaacatacgagccggaagcataaagtgtaaagcctggggtgcctaatgagtgagcta
actcacattaattgcgttgcgctcactgcccgctttccagtcgggaaacctgtcgtgccagctg
cattaatgaatcggccaacgcgcggggagaggcggtttgcgtattgggcgctcttccgcttcct
cgctcactgactcgctgcgctcggtcgttcggctgcggcgagcggtatcagctcactcaaaggc
ggtaatacggttatccacagaatcaggggataacgcaggaaagaacatgtgagcaaaaggccag
caaaaggccaggaaccgtaaaaaggccgcgttgctggcgtttttccataggctccgcccccctg
acgagcatcacaaaaatcgacgctcaagtcagaggtggcgaaacccgacaggactataaagata
ccaggcgtttccccctggaagctccctcgtgcgctctcctgttccgaccctgccgcttaccgga
tacctgtccgcctttctcccttcgggaagcgtggcgctttctcatagctcacgctgtaggtatc
tcagttcggtgtaggtcgttcgctccaagctgggctgtgtgcacgaaccccccgttcagcccga
ccgctgcgccttatccggtaactatcgtcttgagtccaacccggtaagacacgacttatcgcca
ctggcagcagccactggtaacaggattagcagagcgaggtatgtaggcggtgctacagagttct
tgaagtggtggcctaactacggctacactagaagaacagtatttggtatctgcgctctgctgaa
gccagttaccttcggaaaaagagttggtagctcttgatccggcaaacaaaccaccgctggtagc
ggtggtttttttgtttgcaagcagcagattacgcgcagaaaaaaaggatctcaagaagatcctt
tgatcttttctacggggtctgacgctcagtggaacgaaaactcacgttaagggattttggtcat
gagattatcaaaaaggatcttcacctagatccttttaaattaaaaatgaagttttaaatcaatc
taaagtatatatgagtaaacttggtctgacagttaccaatgcttaatcagtgaggcacctatct
cagcgatctgtctatttcgttcatccatagttgcctgactccccgtcgtgtagataactacgat
acgggagggcttaccatctggccccagtgctgcaatgataccgcgagacccacgctcaccggct
ccagatttatcagcaataaaccagccagccggaagggccgagcgcagaagtggtcctgcaactt
tatccgcctccatccagtctattaattgttgccgggaagctagagtaagtagttcgccagttaa
tagtttgcgcaacgttgttgccattgctacaggcatcgtggtgtcacgctcgtcgtttggtatg
gcttcattcagctccggttcccaacgatcaaggcgagttacatgatcccccatgttgtgcaaaa
aagcggttagctccttcggtcctccgatcgttgtcagaagtaagttggccgcagtgttatcact
catggttatggcagcactgcataattctcttactgtcatgccatccgtaagatgcttttctgtg
actggtgagtactcaaccaagtcattctgagaatagtgtatgcggcgaccgagttgctcttgcc
cggcgtcaatacgggataataccgcgccacatagcagaactttaaaagtgctcatcattggaaa
acgttcttcggggcgaaaactctcaaggatcttaccgctgttgagatccagttcgatgtaaccc
actcgtgcacccaactgatcttcagcatcttttactttcaccagcgtttctgggtgagcaaaaa
caggaaggcaaaatgccgcaaaaaagggaataagggcgacacggaaatgttgaatactcatact
cttcctttttcaatattattgaagcatttatcagggttattgtctcatgagcggatacatattt
gaatgtatttagaaaaataaacaaataggggttccgcgcacatttccccgaaaagtgccacctg
acgtcgacggatcgggagatctcccgatcccctatggtgcactctcagtacaatctgctctgat
gccgcatagttaagccagtatctgctccctgcttgtgtgttggaggtcgctgagt

According to some embodiments, p132_Expr_pcDNACBA-C9-AI-stop-His-HA-WPRE-pA_6-FP-CBA-01 (775 bp) comprises SEQ ID NO: 66, shown below.

NNNNNNNNNNNNNNNNNNNNNNCANGTTCTGCCTTCTTCTTTNTCCTACA
GCTCCTGGGCAACGCCACCATGGACAACTTTGTATACAAAAGTTGTAGCC
ACCATGTCGACTCTTTGCCCACCGCCATCTCCAGCTGTTGCCAAGACAGA
GATTGCTTTAAGTGGCAAATCACCTTTATTAGCAGCTACTTTTGCTTACT
GGGACAATATTCTTGGTCCTAGAGTAAGGCACATTTGGGCTCCAAAGACA
GAACAGGTACTTCTCAGTGATGGAGAAATAACTTTTCTTGCCAACCACAC
TCTAAATGGAGAAATCCTTCGAAATGCAGAGAGTGGTGCTATAGATGTAA
AGTTTTTTGTCTTGTCTGAAAAGGGAGTGATTATTGTTTCATTAATCTTT
GATGGAAACTGGAATGGGGATCGCAGCACATATGGACTATCAATTATACT
TCCACAGACAGAACTTAGTTTCTACCTCCCACTTCATAGAGTGTGTGTTG
ATAGATTAACACATATAATCCGGAAAGGAAGAATATGGATGCATAAGGAA
AGACAAGAAAATGTCCAGAAGATTATCTTAAAAGGCACAGAGAGAATGGA
AGATCAGGGTCAGAGTATTATTTCCAATGCTTACTGGAGAAGTGATTCCT
GTAATGGAACTGCTTTCATCTATGAAATCACACAGTGTTCCTGAAGAAAT
AGATATAGCTGATACAGTACTCAATGATGATGATATTGNNGACAGCTGTC
ATGAAGGCTTTCTTTCNNCGNAAGT

According to some embodiments, p132_Expr_pcDNACBA-C9-AI-stop-His-HA-WPRE-pA_6-RP-WPRE-01 (601 bp) comprises SEQ ID NO: 67, shown below.

NNNNNNNNNNNNNNNNNNNTNNAGCAGCGTATCCACATAGCGTAAAAGGA
GCAACATAGTTAAGAATACCAGTCAATCTTTCACAAATTTTGTAATCCAG
AGGTTGATTTCGATAACTTCGTATAGGATACTTTATACGAAGTTATTGCG
ATTCGGATAACTTCGTATAGCATACATTATACGAAGTTATCAAGGCTACA
ACTTTATTATACAAAGTTGTTTAGGCGTAGTCGGGCACGTCGTAGGGGTA
GTGGTGGTGGTGGTGNCCNCCNTGNACANAATCTACTGTATCACCANAAG
ANGNNCCATGGCCATGGNCGAACTCANAATGTCTGATGGGGCAGAACANC
TTCATCNACANCTTCCNACTGCTCACCANANTNNNAAGCCTGTGNACNNN
NNACCCCAAGACCATAATACTGNTGAACGTGCCCCTGCNCCNACCATCCT
GACCANACCCCTGCTNNANACCNANNTANNNATCNNNNCCCTAATCCTGA
NATGCCANGAGAGAATCTCTCCCCACCACCTGNACAGATGCCACAGCCAG
GACCTACCCCAGGAAATGNCCNNTGCCACCANCNTAACCTTTNNNCTACT
A

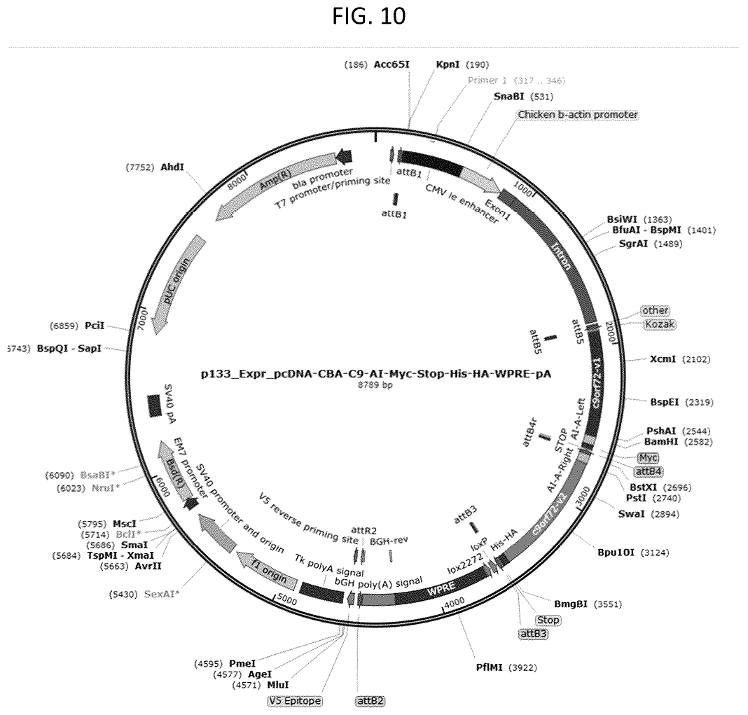

(6) p133_Expr_pcDNA-CBA-C9-AI-Myc-Stop-His-HA-WPRE-pA. This construct comprises CBA promoter, bGH polyA signal, Ampicillin resistance gene. This construct carry a C9orf72 sequence designed to express long C9orf72 protein isoform tagged with His and HA, a short C90rf72 protein isoform tagged with Myc tag The vector map is shown in FIG. 10. According to some embodiments, the nucleic acid sequence of p133_Expr_pcDNA-CBA-C9-AI-Myc-Stop-His-HA-WPRE-pA comprises SEQ ID NO: 68. According to some embodiments, the nucleic acid sequence of p133_Expr_pcDNA-CBA-C9-AI-Myc-Stop-His-HA-WPRE-pA is at least 85%, at least 90%, at least 95%, at least 96%, at least 97%, at least 98%, at least 99% identical to SEQ ID NO: 68, shown below.

agtgcgcgagcaaaatttaagctacaacaaggcaaggcttgaccgacaattctctggctaacta
gagaacccactgcttactggcttatcgaaattaatacgactcactatagggagacccaagctgg
ctagttaagctatcaacaagtttGTACAAAAAAGCAGGCTTActcagatctgaattcggtacct
agttattaatagtaatcaattacggggtcattagttcatagcccatatatggagttccgcgtta
cataacttacggtaaatggcccgcctggctgaccgcccaacgacccccgcccattgacgtcaat
aatgacgtatgttcccatagtaacgccaatagggactttccattgacgtcaatgggtggagtat
ttacggtaaactgcccacttggcagtacatcaagtgtatcatatgccaagtacgccccctattg
acgtcaatgacggtaaatggcccgcctggcattatgcccagtacatgaccttatgggactttcc
tacttggcagtacatctacgtattagtcatcgctattaccatggtcgaggtgagccccacgttc
tgcttcactctccccatctcccccccctccccacccccaattttgtatttatttattttttaat
tattttgtgcagcgatgggggcggggggggggggggggcgcgcgccaggcggggcggggcgggg
cgaggggcggggcggggcgaggcggagaggtgcggcggcagccaatcagagcggcgcgctccga
aagtttccttttatggcgaggcggcggcggcggcggccctataaaaagcgaagcgcgcggcggg
cgggagtcgctgcgcgctgccttcgccccgtgccccgctccgccgccgcctcgcgccgcccgcc
ccggctctgactgaccgcgttactcccacaggtgagcgggcgggacggcccttctcctccgggc
tgtaattagcgcttggtttaatgacggcttgtttcttttctgtggctgcgtgaaagccttgagg
ggctccgggagggccctttgtgcggggggagcggctcggggggtgcgtgcgtgtgtgtgtgcgt
ggggagcgccgcgtgcggctccgcgctgcccggcggctgtgagcgctgcgggcgcggcgcgggg
ctttgtgcgctccgcagtgtgcgcgaggggagcgcggccgggggcggtgccccgcggtgcgggg
ggggctgcgaggggaacaaaggctgcgtgcggggtgtgtgcgtgggggggtgagcagggggtgt
gggcgcgtcggtcgggctgcaaccccccctgcacccccctccccgagttgctgagcacggcccg
gcttcgggtgcggggctccgtacggggcgtggcgcggggctcgccgtgccgggcggggggtggc
ggcaggtgggggtgccgggcggggcggggccgcctcgggccggggagggctcgggggaggggcg
cggcggcccccggagcgccggcggctgtcgaggcgcggcgagccgcagccattgccttttatgg
taatcgtgcgagagggcgcagggacttcctttgtcccaaatctgtgcggagccgaaatctggga
ggcgccgccgcaccccctctagcgggcgcggggcgaagcggtgcggcgccggcaggaaggaaat
gggcggggagggccttcgtgcgtcgccgcgccgccgtccccttctccctctccagcctcggggc
tgtccgcggggggacggctgccttcgggggggacggggcagggcggggttcggcttctggcgtg
tgaccggcggctctagagcctctgctaaccatgttcatgccttcttctttttcctacagctcct
gggcaacgccaccatggACAACTTTGTATACAAAAGTTGTAgccaccATGTCGACTCTTTGCCC
ACCGCCATCTCCAGCTGTTGCCAAGACAGAGATTGCTTTAAGTGGCAAATCACCTTTATTAGCA
GCTACTTTTGCTTACTGGGACAATATTCTTGGTCCTAGAGTAAGGCACATTTGGGCTCCAAAGA
CAGAACAGGTACTTCTCAGTGATGGAGAAATAACTTTTCTTGCCAACCACACTCTAAATGGAGA
AATCCTTCGAAATGCAGAGAGTGGTGCTATAGATGTAAAGTTTTTTGTCTTGTCTGAAAAGGGA
GTGATTATTGTTTCATTAATCTTTGATGGAAACTGGAATGGGGATCGCAGCACATATGGACTAT
CAATTATACTTCCACAGACAGAACTTAGTTTCTACCTCCCACTTCATAGAGTGTGTGTTGATAG
ATTAACACATATAATCCGGAAAGGAAGAATATGGATGCATAAGGAAAGACAAGAAAATGTCCAG
AAGATTATCTTAGAAGGCACAGAGAGAATGGAAGATCAGGGTCAGAGTATTATTCCAATGCTTA
CTGGAGAAGTGATTCCTGTAATGGAACTGCTTTCATCTATGAAATCACACAGTGTTCCTGAAGA
AATAGATATAGCTGATACAGTACTCAATGATGATGATATTGGTGACAGCTGTCATGAAGGCTTT
CTTCTCgtaagtcgactcgttggatccccactacagccgatactcaagcttgacgaattcgacG
AGCAGAAGCTGATCTCCGAGGAGGACCTGTGACCACCCAACTTTTCTATACAAAGTTGTAgtat
ccaaggtagtggactagtgtgacgctgctgacccctttctttcccttctgcagAATGCCATCAG
CTCACACTTGCAAACCTGTGGCTGTTCCGTTGTAGTAGGTAGCAGTGCAGAGAAAGTAAATAAG
ATAGTCAGAACATTATGCCTTTTTCTGACTCCAGCAGAGAGAAAATGCTCCAGGTTATGTGAAG
CAGAATCATCATTTAAATATGAGTCAGGGCTCTTTGTACAAGGCCTGCTAAAGGATTCAACTGG
AAGCTTTGTGCTGCCTTTCCGGCAAGTCATGTATGCTCCATATCCCACCACACACATAGATGTG
GATGTCAATACTGTGAAGCAGATGCCACCCTGTCATGAACATATTTATAATCAGCGTAGATACA
TGAGATCCGAGCTGACAGCCTTCTGGAGAGCCACTTCAGAAGAAGACATGGCTCAGGATACGAT
CATCTACACTGACGAAAGCTTTACTCCTGATTTGAATATTTTTCAAGATGTCTTACACAGAGAC
ACTCTAGTGAAAGCCTTCCTGGATCAGGTCTTTCAGCTGAAACCTGGCTTATCTCTCAGAAGTA
CTTTCCTTGCACAGTTTCTACTTGTCCTTCACAGAAAAGCCTTGACACTAATAAAATATATAGA
AGACGATACGCAGAAGGGAAAAAAGCCCTTTAAATCTCTTCGGAACCTGAAGATAGACCTTGAT
TTAACAGCAGAGGGCGATCTTAACATAATAATGGCTCTGGCTGAGAAAATTAAACCAGGCCTAC
ACTCTTTTATCTTTGGAAGACCTTTCTACACTAGTGTGCAAGAACGAGATGTTCTAATGACTTT
TCACCACCACCACCACCACTACCCCTACGACGTGCCCGACTACGCCTAAACAACTTTGTATAAT
AAAGTTGTAgccttgataacttcgtataatgtatgctatacgaagttatccgaatcgcaataac
ttcgtataaagtatcctatacgaagttatcgaaatcaacctctggattacaaaatttgtgaaag
attgactggtattcttaactatgttgctccttttacgctatgtggatacgctgctttaatgcct
ttgtatcatgctattgcttcccgtatggctttcattttctcctccttgtataaatcctggttgc
tgtctctttatgaggagttgtggcccgttgtcaggcaacgtggcgtggtgtgcactgtgtttgc
tgacgcaacccccactggttggggcattgccaccacctgtcagctcctttccgggactttcgct
ttccccctccctattgccacggcggaactcatcgccgcctgccttgcccgctgctggacagggg
ctcggctgttgggcactgacaattccgtggtgttgtcggggaaatcatcgtcctttccttggct
gctcgcctgtgttgccacctggattctgcgcgggacgtccttctgctacgtcccttcggccctc
aatccagcggaccttccttcccgcggcctgctgccggctctgcggcctcttccgcgtcttcgcc
ttcgccctcagacgagtcggatctccctttgggccgcctccccgcctgctgtgccttctagttg
ccagccatctgttgtttgcccctcccccgtgccttccttgaccctggaaggtgccactcccact
gtcctttcctaataaaatgaggaaattgcatcgcattgtctgagtaggtgtcattctattctgg
ggggtggggtggggcaggacagcaagggggaggattgggaagacaatagcaggcatgctgggga
AACCCAGCTTTcttgtacaaagtggttgatctagagggcccgcggttcgaaggtaagcctatcc
ctaaccctctcctcggtctcgattctacgcgtaccggttagtaatgagtttaaacgggggaggc
taactgaaacacggaaggagacaataccggaaggaacccgcgctatgacggcaataaaaagaca
gaataaaacgcacgggtgttgggtcgtttgttcataaacgcggggttcggtcccagggctggca
ctctgtcgataccccaccgagaccccattggggccaatacgcccgcgtttcttccttttcccca
ccccaccccccaagttcgggtgaaggcccagggctcgcagccaacgtcggggcggcaggccctg
ccatagcagatctgcgcagctggggctctagggggtatccccacgcgccctgtagcggcgcatt
aagcgcggcgggtgtggtggttacgcgcagcgtgaccgctacacttgccagcgccctagcgccc
gctcctttcgctttcttcccttcctttctcgccacgttcgccggctttccccgtcaagctctaa
atcgggggctccctttagggttccgatttagtgctttacggcacctcgaccccaaaaaacttga
ttagggtgatggttcacgtagtgggccatcgccctgatagacggtttttcgccctttgacgttg
gagtccacgttctttaatagtggactcttgttccaaactggaacaacactcaaccctatctcgg
tctattcttttgatttataagggattttgccgatttcggcctattggttaaaaaatgagctgat
ttaacaaaaatttaacgcgaattaattctgtggaatgtgtgtcagttagggtgtggaaagtccc
caggctccccagcaggcagaagtatgcaaagcatgcatctcaattagtcagcaaccaggtgtgg
aaagtccccaggctccccagcaggcagaagtatgcaaagcatgcatctcaattagtcagcaacc
atagtcccgcccctaactccgcccatcccgcccctaactccgcccagttccgcccattctccgc
cccatggctgactaattttttttatttatgcagaggccgaggccgcctctgcctctgagctatt
ccagaagtagtgaggaggcttttttggaggcctaggcttttgcaaaaagctcccgggagcttgt
atatccattttcggatctgatcagcacgtgttgacaattaatcatcggcatagtatatcggcat
agtataatacgacaaggtgaggaactaaaccatggccaagcctttgtctcaagaagaatccacc
ctcattgaaagagcaacggctacaatcaacagcatccccatctctgaagactacagcgtcgcca
gcgcagctctctctagcgacggccgcatcttcactggtgtcaatgtatatcattttactggggg
accttgtgcagaactcgtggtgctgggcactgctgctgctgcggcagctggcaacctgacttgt
atcgtcgcgatcggaaatgagaacaggggcatcttgagcccctgcggacggtgccgacaggtgc
ttctcgatctgcatcctgggatcaaagccatagtgaaggacagtgatggacagccgacggcagt
tgggattcgtgaattgctgccctctggttatgtgtgggagggctaagcacttcgtggccgagga
gcaggactgacacgtgctacgagatttcgattccaccgccgccttctatgaaaggttgggcttc
ggaatcgttttccgggacgccggctggatgatcctccagcgcggggatctcatgctggagttct
tcgcccaccccaacttgtttattgcagcttataatggttacaaataaagcaatagcatcacaaa
tttcacaaataaagcatttttttcactgcattctagttgtggtttgtccaaactcatcaatgta
tcttatcatgtctgtataccgtcgacctctagctagagcttggcgtaatcatggtcatagctgt
ttcctgtgtgaaattgttatccgctcacaattccacacaacatacgagccggaagcataaagtg
taaagcctggggtgcctaatgagtgagctaactcacattaattgcgttgcgctcactgcccgct
ttccagtcgggaaacctgtcgtgccagctgcattaatgaatcggccaacgcgcggggagaggcg
gtttgcgtattgggcgctcttccgcttcctcgctcactgactcgctgcgctcggtcgttcggct
gcggcgagcggtatcagctcactcaaaggcggtaatacggttatccacagaatcaggggataac
gcaggaaagaacatgtgagcaaaaggccagcaaaaggccaggaaccgtaaaaaggccgcgttgc
tggcgtttttccataggctccgcccccctgacgagcatcacaaaaatcgacgctcaagtcagag
gtggcgaaacccgacaggactataaagataccaggcgtttccccctggaagctccctcgtgcgc
tctcctgttccgaccctgccgcttaccggatacctgtccgcctttctcccttcgggaagcgtgg
cgctttctcatagctcacgctgtaggtatctcagttcggtgtaggtcgttcgctccaagctggg
ctgtgtgcacgaaccccccgttcagcccgaccgctgcgccttatccggtaactatcgtcttgag
tccaacccggtaagacacgacttatcgccactggcagcagccactggtaacaggattagcagag
cgaggtatgtaggcggtgctacagagttcttgaagtggtggcctaactacggctacactagaag
aacagtatttggtatctgcgctctgctgaagccagttaccttcggaaaaagagttggtagctct
tgatccggcaaacaaaccaccgctggtagcggtggtttttttgtttgcaagcagcagattacgc
gcagaaaaaaaggatctcaagaagatcctttgatcttttctacggggtctgacgctcagtggaa
cgaaaactcacgttaagggattttggtcatgagattatcaaaaaggatcttcacctagatcctt
ttaaattaaaaatgaagttttaaatcaatctaaagtatatatgagtaaacttggtctgacagtt
accaatgcttaatcagtgaggcacctatctcagcgatctgtctatttcgttcatccatagttgc
ctgactccccgtcgtgtagataactacgatacgggagggcttaccatctggccccagtgctgca
atgataccgcgagacccacgctcaccggctccagatttatcagcaataaaccagccagccggaa
gggccgagcgcagaagtggtcctgcaactttatccgcctccatccagtctattaattgttgccg
ggaagctagagtaagtagttcgccagttaatagtttgcgcaacgttgttgccattgctacaggc
atcgtggtgtcacgctcgtcgtttggtatggcttcattcagctccggttcccaacgatcaaggc
gagttacatgatcccccatgttgtgcaaaaaagcggttagctccttcggtcctccgatcgttgt
cagaagtaagttggccgcagtgttatcactcatggttatggcagcactgcataattctcttact
gtcatgccatccgtaagatgcttttctgtgactggtgagtactcaaccaagtcattctgagaat
agtgtatgcggcgaccgagttgctcttgcccggcgtcaatacgggataataccgcgccacatag
cagaactttaaaagtgctcatcattggaaaacgttcttcggggcgaaaactctcaaggatctta
ccgctgttgagatccagttcgatgtaacccactcgtgcacccaactgatcttcagcatctttta
ctttcaccagcgtttctgggtgagcaaaaacaggaaggcaaaatgccgcaaaaaagggaataag
ggcgacacggaaatgttgaatactcatactcttcctttttcaatattattgaagcatttatcag
ggttattgtctcatgagcggatacatatttgaatgtatttagaaaaataaacaaataggggttc
cgcgcacatttccccgaaaagtgccacctgacgtcgacggatcgggagatctcccgatccccta
tggtgcactctcagtacaatctgctctgatgccgcatagttaagccagtatctgctccctgctt
gtgtgttggaggtcgctgagt

According to some embodiments, p133_Expr_pcDNA-CBA-C9-AI-Myc-Stop-His-HA-WPRE-pA_1-FP-CBA-01 (1086 bp) comprises SEQ ID NO: 69, shown below.

NNNNNNNNNNNNNNNNNNNNNNNNNNGNNCTNCCTTCTTCTTTTTCCTAC
AGCTCCTGGGCAACGCCACCATGGACAACTTTGTATACAAAAGTTGTAGC
CACCATGTCGACTCTTTGCCCACCGCCATCTCCAGCTGTTGCCAAGACAG
AGATTGCTTTAAGTGGCAAATCACCTTTATTAGCAGCTACTTTTGCTTAC
TGGGACAATATTCTTGGTCCTAGAGTAAGGCACATTTGGGCTCCAAAGAC
AGAACAGGTACTTCTCAGTGATGGAGAAATAACTTTTCTTGCCAACCACA
CTCTAAATGGAGAAATCCTTCGAAATGCAGAGAGTGGTGCTATAGATGTA
AAGTTTTTTGTCTTGTCTGAAAAGGGAGTGATTATTGTTTCATTAATCTT
TGATGGAAACTGGAATGGGGATCGCAGCACATATGGACTATCAATTATAC
TTCCACAGACAGAACTTAGTTTCTACCTCCCACTTCATAGAGTGTGTGTT
GATAGATTAACACATATAATCCGGAAAGGAAGAATATGGATGCATAAGGA
AAGACAAGAAAATGTCCAGAAGATTATCTTAGAAGGCACAGAGAGAATGG
AAGATCAGGGTCAGAGTATTATTCCAATGCTTACTGGAGAAGTGATTCCT
GTAATGGAACTGCTTTCATCTATGAAATCACACAGTGTTCCTGAAGAAAT
AGATATAGCTGATACAGTACTCAATGATGATGATATTGGTGACAGCTGTC
ATGAAGGCTTTCTTCTCGTAAGTCGACTCGTTGGATCCCCACTACAGCCG
ATACTCAAGCTTGACGAATTCGACGAGCAGAAGCTGATCTCCGANGAGGA
CCTGTGACCACCCAACTTTTCTATACAAAGTTGTAGTATCCAAGGTAGTG
GACTAGNGTGACGCTGCTGACCCCTTTCNTTTCCCTTCTGCAGAATGCCA
TCAGCTCACACTTGCAAACCTGTGGCTGTTCCGTTGTAGTNGGTAGCAGT
GCANANAAAGTAAATAANANAGTCNNAACATTATGCCTTTTTCTGANTTC
CNGCANANANAAANGNNCCAGGTTNNNNNNGAANNN

According to some embodiments, p133_Expr_pcDNA-CBA-C9-AI-Myc-Stop-His-HA-WPRE-pA_1-RP-WPRE-01 (938 bp) comprises SEQ ID NO: 70, shown below.

NNNNNNNNNNNNNGNATNNNNNAGCGTATCCACATAGCGTAAAAGGAGCA
ACATAGTTAAGAATACCAGTCAATCTTTCACAAATTTTGTAATCCAGAGG
TTGATTTCGATAACTTCGTATAGGATACTTTATACGAAGTTATTGCGATT
CGGATAACTICGTATAGCATACATTATACGAAGTTATCAAGGCTACAACT
TTATTATACAAAGTTGTTTAGGCGTAGTCGGGCACGTCGTAGGGGTAGTG
GTGGTGGTGGTGGTGAAAAGTCATTAGAACATCTCGTTCTTGCACACTAG
TGTAGAAAGGTCTTCCAAAGATAAAAGAGTGTAGGCCTGGTTTAATTTTC
TCAGCCAGAGCCATTATTATGTTAAGATCGCCCTCTGCTGTTAAATCAAG
GTCTATCTTCAGGTTCCGAAGAGATTTAAAGGGCTTTTTTCCCTTCTGCG
TATCGTCTTCTATATATTTTATTAGTGTCAAGGCTTTTCTGTGAAGGACA
AGTAGAAACTGTGCAAGGAAAGTACTTCTGAGAGATAAGCCAGGTTTCAG
CTGAAAGACCTGATCCAGGAAGGCTTTCACTAGAGTGTCTCTGTGTAAGA
CATCTTGAAAAATATTCAAATCAGGAGTAAAGCTTTCGTCAGTGTAGATG
ATCGTATCCTGAGCCATGTCTTCTTCTGAAGTGGCTCTCCAGAAGGCTGT
CAGCTCGGATCTCATGTATCTACGCTGATTATAAATATGTTCATGACAGG
GTGGCATCTGCTTCACAGTATTGACATCCACATCTATGTGTGTGGTGGGA
TATGGAGCATACATGACTTGCCGGAAAGGCAGCACAAAGCTTCCAGTTGA
ATCCTTTTAGCNNGCNTGNACAAAGAGCCCTGACTCATATTNNAATGATG
ANTNNGCTTNNCATNANCCTGGAANCNNTTNCNCTNTG

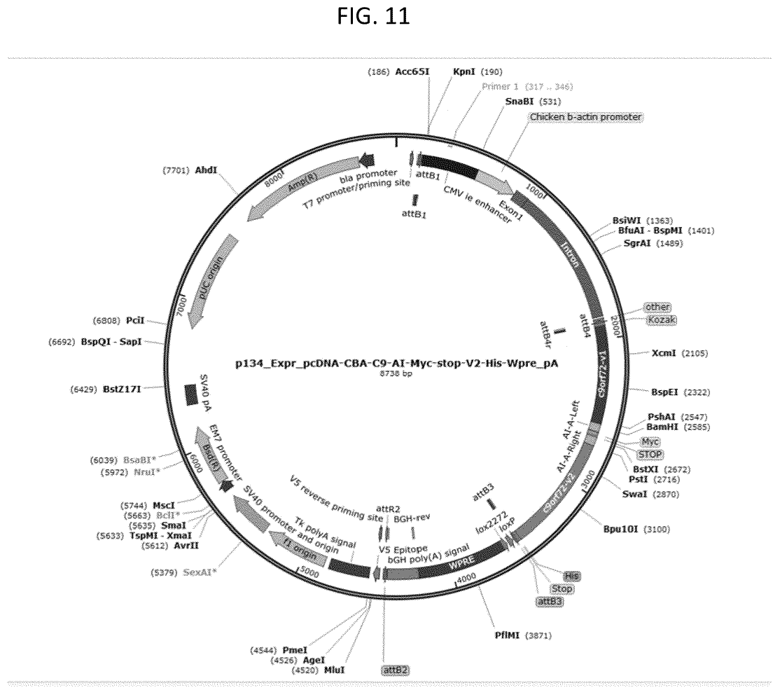

(7) p134_Expr_pcDNA-CBA-C9-AI-Myc-stop-V2-His-Wpre_pA. This construct comprises CBA promoter, bGH polyA signal, Ampicillin resistance gene. This construct carry a C9orf72 sequence designed to express long C9orf72 protein isoform tagged with His, a short C90rf72 protein isoform tagged with Myc tag. The vector map is shown in FIG. 11. According to some embodiments, the nucleic acid sequence of p134_Expr_pcDNA-CBA-C9-AI-Myc-stop-V2-His-Wpre_pA comprises SEQ ID NO: 71. According to some embodiments, the nucleic acid sequence of p134_Expr_pcDNA-CBA-C9-AI-Myc-stop-V2-His-Wpre_pA is at least 85%, at least 90%, at least 95%, at least 96%, at least 97%, at least 98%, at least 99% identical to SEQ ID NO: 71.

agtgcgcgagcaaaatttaagctacaacaaggcaaggcttgaccgacaattctctggctaacta
gagaacccactgcttactggcttatcgaaattaatacgactcactatagggagacccaagctgg
ctagttaagctatcaacaagtttGTACAAAAAAGCAGGCTTActcagatctgaattcggtacct
agttattaatagtaatcaattacggggtcattagttcatagcccatatatggagttccgcgtta
cataacttacggtaaatggcccgcctggctgaccgcccaacgacccccgcccattgacgtcaat
aatgacgtatgttcccatagtaacgccaatagggactttccattgacgtcaatgggtggagtat
ttacggtaaactgcccacttggcagtacatcaagtgtatcatatgccaagtacgccccctattg
acgtcaatgacggtaaatggcccgcctggcattatgcccagtacatgaccttatgggactttcc
tacttggcagtacatctacgtattagtcatcgctattaccatggtcgaggtgagccccacgttc
tgcttcactctccccatctcccccccctccccacccccaattttgtatttatttattttttaat
tattttgtgcagcgatgggggcggggggggggggggggcgcgcgccaggcggggcggggcgggg
cgaggggcggggcggggcgaggcggagaggtgcggcggcagccaatcagagcggcgcgctccga
aagtttccttttatggcgaggcggcggcggcggcggccctataaaaagcgaagcgcgcggcggg
cgggagtcgctgcgcgctgccttcgccccgtgccccgctccgccgccgcctcgcgccgcccgcc
ccggctctgactgaccgcgttactcccacaggtgagcgggcgggacggcccttctcctccgggc
tgtaattagcgcttggtttaatgacggcttgtttcttttctgtggctgcgtgaaagccttgagg
ggctccgggagggccctttgtgcggggggagcggctcggggggtgcgtgcgtgtgtgtgtgcgt
ggggagcgccgcgtgcggctccgcgctgcccggcggctgtgagcgctgcgggcgcggcgcgggg
ctttgtgcgctccgcagtgtgcgcgaggggagcgcggccgggggcggtgccccgcggtgcgggg
ggggctgcgaggggaacaaaggctgcgtgcggggtgtgtgcgtgggggggtgagcagggggtgt
gggcgcgtcggtcgggctgcaaccccccctgcacccccctccccgagttgctgagcacggcccg
gcttcgggtgcggggctccgtacggggcgtggcgcggggctcgccgtgccgggcggggggtggc
ggcaggtgggggtgccgggcggggcggggccgcctcgggccggggagggctcgggggaggggcg
cggcggcccccggagcgccggcggctgtcgaggcgcggcgagccgcagccattgccttttatgg
taatcgtgcgagagggcgcagggacttcctttgtcccaaatctgtgcggagccgaaatctggga
ggcgccgccgcaccccctctagcgggcgcggggcgaagcggtgcggcgccggcaggaaggaaat
gggcggggagggccttcgtgcgtcgccgcgccgccgtccccttctccctctccagcctcggggc
tgtccgcggggggacggctgccttcgggggggacggggcagggcggggttcggcttctggcgtg
tgaccggcggctctagagcctctgctaaccatgttcatgccttcttctttttcctacagctcct
gggcaacgccaccatggCACCCAACTTTTCTATACAAAGTTGTAgccaccATGTCGACTCTTTG
CCCACCGCCATCTCCAGCTGTTGCCAAGACAGAGATTGCTTTAAGTGGCAAATCACCTTTATTA
GCAGCTACTTTTGCTTACTGGGACAATATTCTTGGTCCTAGAGTAAGGCACATTTGGGCTCCAA
AGACAGAACAGGTACTTCTCAGTGATGGAGAAATAACTTTTCTTGCCAACCACACTCTAAATGG
AGAAATCCTTCGAAATGCAGAGAGTGGTGCTATAGATGTAAAGTTTTTTGTCTTGTCTGAAAAG
GGAGTGATTATTGTTTCATTAATCTTTGATGGAAACTGGAATGGGGATCGCAGCACATATGGAC
TATCAATTATACTTCCACAGACAGAACTTAGTTTCTACCTCCCACTTCATAGAGTGTGTGTTGA
TAGATTAACACATATAATCCGGAAAGGAAGAATATGGATGCATAAGGAAAGACAAGAAAATGTC
CAGAAGATTATCTTAGAAGGCACAGAGAGAATGGAAGATCAGGGTCAGAGTATTATTCCAATGC
TTACTGGAGAAGTGATTCCTGTAATGGAACTGCTTTCATCTATGAAATCACACAGTGTTCCTGA
AGAAATAGATATAGCTGATACAGTACTCAATGATGATGATATTGGTGACAGCTGTCATGAAGGC
TTTCTTCTCgtaagtcgactcgttggatccccactacagccgatactcaagcttgacgaattcg
acGAGCAGAAGCTGATCTCCGAGGAGGACCTGTGACgtatccaaggtagtggactagtgtgacg
ctgctgacccctttctttcccttctgcagAATGCCATCAGCTCACACTTGCAAACCTGTGGCTG
TTCCGTTGTAGTAGGTAGCAGTGCAGAGAAAGTAAATAAGATAGTCAGAACATTATGCCTTTTT
CTGACTCCAGCAGAGAGAAAATGCTCCAGGTTATGTGAAGCAGAATCATCATTTAAATATGAGT
CAGGGCTCTTTGTACAAGGCCTGCTAAAGGATTCAACTGGAAGCTTTGTGCTGCCTTTCCGGCA
AGTCATGTATGCTCCATATCCCACCACACACATAGATGTGGATGTCAATACTGTGAAGCAGATG
CCACCCTGTCATGAACATATTTATAATCAGCGTAGATACATGAGATCCGAGCTGACAGCCTTCT
GGAGAGCCACTTCAGAAGAAGACATGGCTCAGGATACGATCATCTACACTGACGAAAGCTTTAC
TCCTGATTTGAATATTTTTCAAGATGTCTTACACAGAGACACTCTAGTGAAAGCCTTCCTGGAT
CAGGTCTTTCAGCTGAAACCTGGCTTATCTCTCAGAAGTACTTTCCTTGCACAGTTTCTACTTG
TCCTTCACAGAAAAGCCTTGACACTAATAAAATATATAGAAGACGATACGCAGAAGGGAAAAAA
GCCCTTTAAATCTCTTCGGAACCTGAAGATAGACCTTGATTTAACAGCAGAGGGCGATCTTAAC
ATAATAATGGCTCTGGCTGAGAAAATTAAACCAGGCCTACACTCTTTTATCTTTGGAAGACCTT
TCTACACTAGTGTGCAAGAACGAGATGTTCTAATGACTTTTCACCACCACCACCACCACTAAAC
AACTTTGTATAATAAAGTTGTAgccttgataacttcgtataatgtatgctatacgaagttatcc
gaatcgcaataacttcgtataaagtatcctatacgaagttatcgaaatcaacctctggattaca
aaatttgtgaaagattgactggtattcttaactatgttgctccttttacgctatgtggatacgc
tgctttaatgcctttgtatcatgctattgcttcccgtatggctttcattttctcctccttgtat
aaatcctggttgctgtctctttatgaggagttgtggcccgttgtcaggcaacgtggcgtggtgt
gcactgtgtttgctgacgcaacccccactggttggggcattgccaccacctgtcagctcctttc
cgggactttcgctttccccctccctattgccacggcggaactcatcgccgcctgccttgcccgc
tgctggacaggggctcggctgttgggcactgacaattccgtggtgttgtcggggaaatcatcgt
cctttccttggctgctcgcctgtgttgccacctggattctgcgcgggacgtccttctgctacgt
cccttcggccctcaatccagcggaccttccttcccgcggcctgctgccggctctgcggcctctt
ccgcgtcttcgccttcgccctcagacgagtcggatctccctttgggccgcctccccgcctgctg
tgccttctagttgccagccatctgttgtttgcccctcccccgtgccttccttgaccctggaagg
tgccactcccactgtcctttcctaataaaatgaggaaattgcatcgcattgtctgagtaggtgt
cattctattctggggggtggggtggggcaggacagcaagggggaggattgggaagacaatagca
ggcatgctggggaAACCCAGCTTTcttgtacaaagtggttgatctagagggcccgcggttcgaa
ggtaagcctatccctaaccctctcctcggtctcgattctacgcgtaccggttagtaatgagttt
aaacgggggaggctaactgaaacacggaaggagacaataccggaaggaacccgcgctatgacgg
caataaaaagacagaataaaacgcacgggtgttgggtcgtttgttcataaacgcggggttcggt
cccagggctggcactctgtcgataccccaccgagaccccattggggccaatacgcccgcgtttc
ttccttttccccaccccaccccccaagttcgggtgaaggcccagggctcgcagccaacgtcggg
gcggcaggccctgccatagcagatctgcgcagctggggctctagggggtatccccacgcgccct
gtagcggcgcattaagcgcggcgggtgtggtggttacgcgcagcgtgaccgctacacttgccag
cgccctagcgcccgctcctttcgctttcttcccttcctttctcgccacgttcgccggctttccc
cgtcaagctctaaatcgggggctccctttagggttccgatttagtgctttacggcacctcgacc
ccaaaaaacttgattagggtgatggttcacgtagtgggccatcgccctgatagacggtttttcg
ccctttgacgttggagtccacgttctttaatagtggactcttgttccaaactggaacaacactc
aaccctatctcggtctattcttttgatttataagggattttgccgatttcggcctattggttaa
aaaatgagctgatttaacaaaaatttaacgcgaattaattctgtggaatgtgtgtcagttaggg
tgtggaaagtccccaggctccccagcaggcagaagtatgcaaagcatgcatctcaattagtcag
caaccaggtgtggaaagtccccaggctccccagcaggcagaagtatgcaaagcatgcatctcaa
ttagtcagcaaccatagtcccgcccctaactccgcccatcccgcccctaactccgcccagttcc
gcccattctccgccccatggctgactaattttttttatttatgcagaggccgaggccgcctctg
cctctgagctattccagaagtagtgaggaggcttttttggaggcctaggcttttgcaaaaagct
cccgggagcttgtatatccattttcggatctgatcagcacgtgttgacaattaatcatcggcat
agtatatcggcatagtataatacgacaaggtgaggaactaaaccatggccaagcctttgtctca
agaagaatccaccctcattgaaagagcaacggctacaatcaacagcatccccatctctgaagac
tacagcgtcgccagcgcagctctctctagcgacggccgcatcttcactggtgtcaatgtatatc
attttactgggggaccttgtgcagaactcgtggtgctgggcactgctgctgctgcggcagctgg
caacctgacttgtatcgtcgcgatcggaaatgagaacaggggcatcttgagcccctgcggacgg
tgccgacaggtgcttctcgatctgcatcctgggatcaaagccatagtgaaggacagtgatggac
agccgacggcagttgggattcgtgaattgctgccctctggttatgtgtgggagggctaagcact
tcgtggccgaggagcaggactgacacgtgctacgagatttcgattccaccgccgccttctatga
aaggttgggcttcggaatcgttttccgggacgccggctggatgatcctccagcgcggggatctc
atgctggagttcttcgcccaccccaacttgtttattgcagcttataatggttacaaataaagca
atagcatcacaaatttcacaaataaagcatttttttcactgcattctagttgtggtttgtccaa
actcatcaatgtatcttatcatgtctgtataccgtcgacctctagctagagcttggcgtaatca
tggtcatagctgtttcctgtgtgaaattgttatccgctcacaattccacacaacatacgagccg
gaagcataaagtgtaaagcctggggtgcctaatgagtgagctaactcacattaattgcgttgcg
ctcactgcccgctttccagtcgggaaacctgtcgtgccagctgcattaatgaatcggccaacgc
gcggggagaggcggtttgcgtattgggcgctcttccgcttcctcgctcactgactcgctgcgct
cggtcgttcggctgcggcgagcggtatcagctcactcaaaggcggtaatacggttatccacaga
atcaggggataacgcaggaaagaacatgtgagcaaaaggccagcaaaaggccaggaaccgtaaa
aaggccgcgttgctggcgtttttccataggctccgcccccctgacgagcatcacaaaaatcgac
gctcaagtcagaggtggcgaaacccgacaggactataaagataccaggcgtttccccctggaag
ctccctcgtgcgctctcctgttccgaccctgccgcttaccggatacctgtccgcctttctccct
tcgggaagcgtggcgctttctcatagctcacgctgtaggtatctcagttcggtgtaggtcgttc
gctccaagctgggctgtgtgcacgaaccccccgttcagcccgaccgctgcgccttatccggtaa
ctatcgtcttgagtccaacccggtaagacacgacttatcgccactggcagcagccactggtaac
aggattagcagagcgaggtatgtaggcggtgctacagagttcttgaagtggtggcctaactacg
gctacactagaagaacagtatttggtatctgcgctctgctgaagccagttaccttcggaaaaag
agttggtagctcttgatccggcaaacaaaccaccgctggtagcggtggtttttttgtttgcaag
cagcagattacgcgcagaaaaaaaggatctcaagaagatcctttgatcttttctacggggtctg
acgctcagtggaacgaaaactcacgttaagggattttggtcatgagattatcaaaaaggatctt
cacctagatccttttaaattaaaaatgaagttttaaatcaatctaaagtatatatgagtaaact
tggtctgacagttaccaatgcttaatcagtgaggcacctatctcagcgatctgtctatttcgtt
catccatagttgcctgactccccgtcgtgtagataactacgatacgggagggcttaccatctgg
ccccagtgctgcaatgataccgcgagacccacgctcaccggctccagatttatcagcaataaac
cagccagccggaagggccgagcgcagaagtggtcctgcaactttatccgcctccatccagtcta
ttaattgttgccgggaagctagagtaagtagttcgccagttaatagtttgcgcaacgttgttgc
cattgctacaggcatcgtggtgtcacgctcgtcgtttggtatggcttcattcagctccggttcc
caacgatcaaggcgagttacatgatcccccatgttgtgcaaaaaagcggttagctccttcggtc
ctccgatcgttgtcagaagtaagttggccgcagtgttatcactcatggttatggcagcactgca
taattctcttactgtcatgccatccgtaagatgcttttctgtgactggtgagtactcaaccaag
tcattctgagaatagtgtatgcggcgaccgagttgctcttgcccggcgtcaatacgggataata
ccgcgccacatagcagaactttaaaagtgctcatcattggaaaacgttcttcggggcgaaaact
ctcaaggatcttaccgctgttgagatccagttcgatgtaacccactcgtgcacccaactgatct
tcagcatcttttactttcaccagcgtttctgggtgagcaaaaacaggaaggcaaaatgccgcaa
aaaagggaataagggcgacacggaaatgttgaatactcatactcttcctttttcaatattattg
aagcatttatcagggttattgtctcatgagcggatacatatttgaatgtatttagaaaaataaa
caaataggggttccgcgcacatttccccgaaaagtgccacctgacgtcgacggatcgggagatc
tcccgatcccctatggtgcactctcagtacaatctgctctgatgccgcatagttaagccagtat
ctgctccctgcttgtgtgttggaggtcgctgagt

According to some embodiments, p134_Expr_pcDNA-CBA-C9-AI-Myc-stop-V2-His-Wpre_pA_1-FP-CBA-01 (936 bp) comprises SEQ ID NO: 72, shown below.

NNNNNNNNNNNNNNNNNNNNNNNNNNNANNTGTNNTGCCTTCTTCTTTTT
CCTACAGCTCCTGGGCAACGCCACCATGGCACCCAACTTTTCTATACAAA
GTTGTAGCCACCATGTCGACTCTTTGCCCACCGCCATCTCCAGCTGTTGC
CAAGACAGAGATTGCTTTAAGTGGCAAATCACCTTTATTAGCAGCTACTT
TTGCTTACTGGGACAATATTCTTGGTCCTAGAGTAAGGCACATTTGGGCT
CCAAAGACAGAACAGGTACTTCTCAGTGATGGAGAAATAACTTTTCTTGC
CAACCACACTCTAAATGGAGAAATCCTTCGAAATGCAGAGAGTGGTGCTA
TAGATGTAAAGTTTTTTGTCTTGTCTGAAAAGGGAGTGATTATTGTTTCA
TTAATCTTTGATGGAAACTGGAATGGGGATCGCAGCACATATGGACTATC
AATTATACTTCCACAGACAGAACTTAGTTTCTACCTCCCACTTCATAGAG
TGTGTGTTGATAGATTAACACATATAATCCGGAAAGGAAGAATATGGATG
CATAAGGAAAGACAAGAAAATGTCCAGAAGATTATCTTAGAAGGCACAGA
GAGAATGGAAGATCAGGGTCAGAGTATTATTCCAATGCTTACTGGAGAAG
TGATTCCTGTAATGGAACTGCTTTCATCTATGAAATCACACAGTGTTCCT
GAAGAAATAGATATAGCTGATACAGTACTCAATGATGATGATATTGGTGA
CAGCTGTCATGAAGGCTTTCTTCTCGTAAGTCGACTCGTTGGATCCCCAC
TACAGCCGATACTCAAGCTTGACGAATTCGACGAGCAGAAGCTGATCTCC
GAGGAGGANCTGTGACGTATCCAAAGGNAGTGGACTAGTGTGACGCTGCT
GACCCCTTTCTTTCCCTTCTGCAGAATGCCATCAGC

According to some embodiments, p134_Expr_pcDNA-CBA-C9-AI-Myc-stop-V2-His-Wpre_pA_1-RP-WPRE-01 (846 bp) comprises SEQ ID NO: 73, shown below.

NNNNNNNNNNNNNNNNNGCATTANAGCAGCGTATCCACATAGCGTAAAAG
GAGCAACATAGTTAAGAATACCAGTCAATCTTTCACNAATTTTGTAATCC
AGAGGTTGATTTCGATAACTTCGTATAGGATACTTTATACGAAGTTATTG
CGATTCGGATAACTTCGTATAGCATACATTATACGAAGTTATCAAGGCTA
CAACTTTATTATACAAAGTTGTTTAGTGGTGGTGGTGGTGGTGAAAAGTC
ATTAGAACATCTCGTTCTTGCACACTAGTGTAGAAAGGTCTTCCAAAGAT
AAAAGAGTGTAGGCCTGGTTTAATTTTCTCAGCCAGAGCCATTATTATGT
TAAGATCGCCCTCTGCTGTTAAATCAAGGTCTATCTTCAGGTTCCGAAGA
GATTTAAAGGGCTTTTTTCCCTTCTGCGTATCGTCTTCTATATATTTTAT
TAGTGTCAAGGCTTTTCTGTGAAGGACAAGTAGAAACTGTGCAAGGAAAG
TACTTCTGAGAGATAAGCCAGGTTTCAGCTGAAAGACCTGATCCAGGAAG
GCTTTCACTAGAGTGTCTCTGTGTAAGACATCTTGAAAAATATTCAAATC
AGGAGTAAAGCTTTCGTCAGTGTAGATGATCGTATCCTGAGCCATGTCTT
CTTCTGAAGTGGCTCTCCAGAAGGCTGTCAGCTCGGATCTCATGTATCTA
CGCTGATTATAAATATGTTCATGACAGGGTGGCATCTGCTTCACAGTATT
GACATCCACATCTATGTGTGTGGTGGGATATGGAGCATACATGACTTGCC
GGAAAGGCAGCACAAAGCTTCCAGTTGAATCCTTTAGCAGGCCTTG

Dynamic Range Control of Gene Expression Levels

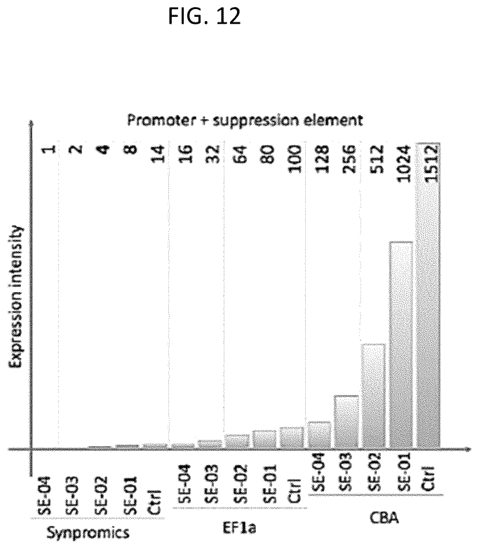

It is possible that over expression of c9orf72 will be toxic, over long term in vivo. Thus, precise expression levels of both v1 & v2 variants are key requirements. A 3D mRNA attenuator (˜200 nt) was used to tune expression levels. This creates a “High Dynamic Range” of expression level control. FIG. 12 is a graph showing the high dynamic range that was generated by different promoters.

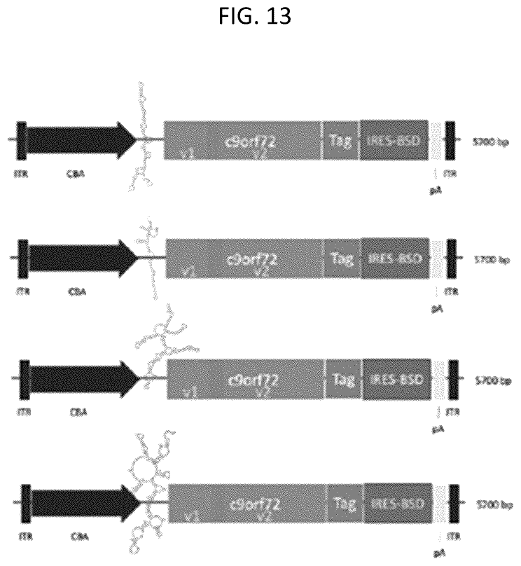

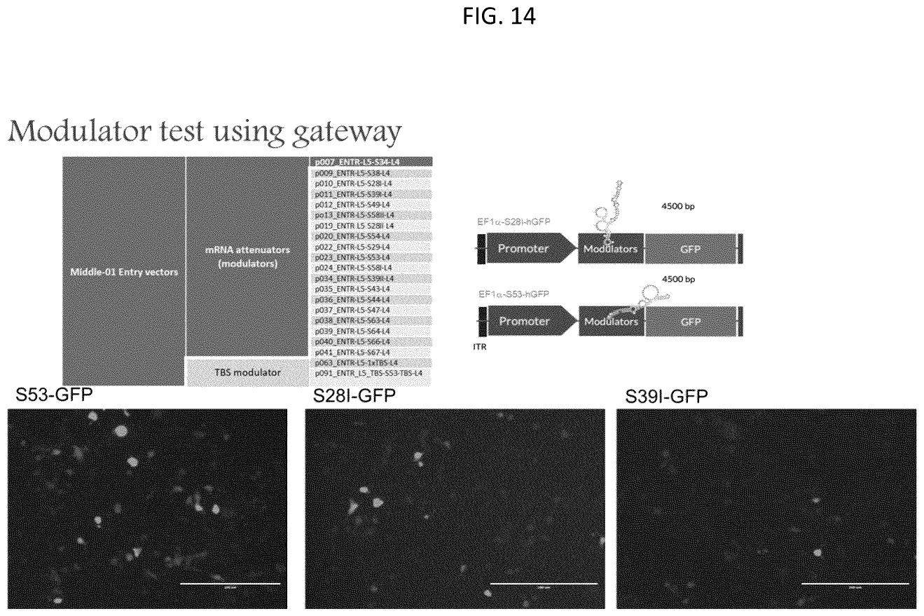

A 3D mRNA attenuator can be placed into the 3′ UTR or in artificial introns. 3′ UTR placement will control the overall expression levels. Artificial intron placement will control the ratio of v1/v2 variants. The promoter used determines the upper and lower boundaries of expressions. FIG. 13 shows schematic constructs and dose ranges. FIG. 14 shows the result of a 3D mRNA attenuator test experiment. From the intensity of the fluorescence, it can be seen that different 3D mRNA attenuators have different influence on the gene's expression level.

### In Vitro Validation in HEK293 Cells

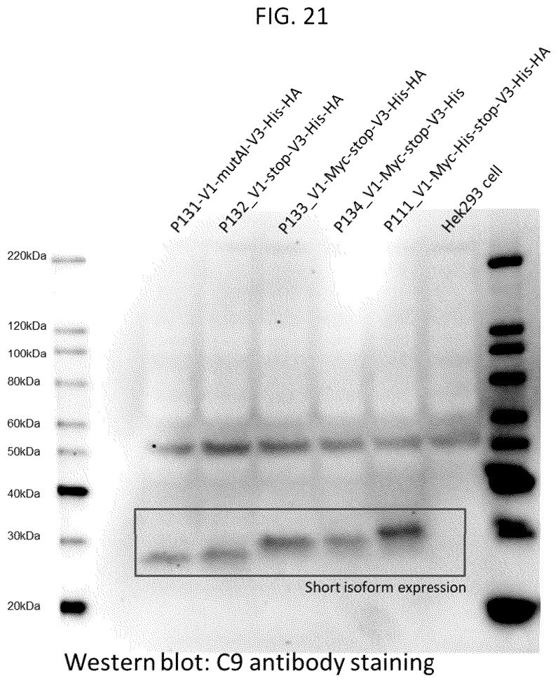

Experiments were performed to detect the expression of C9orf72 protein. Briefly, HEK293 cells were transfected and selected with Puro+ or BSD+, or Hygro+. 48-72 hrs later, Western Blots were prepared. Epitope tags His, cMyc, HA were used for detection. Results are shown in FIG. 21. From this data, it was confirmed that short isoform of C9orf72 protein was successfully expressed.

HEK293 mRNA Sequencing Data

Both 1 and V2 variant mRNA should be detected

V1 variant mRNA length is expected to be ˜3,795 bp (including IVS: 960 bp).

V2 variant mRNA length is expected to be ˜2,835 bp (excluding IVS: 960 bp).

### HEK293 IHC Staining Data

In a set of experiments, expression of the V1 and V2 variants will be determined in HEK293 cells in vitro using immunohistochemistry. V1 will be detected by cMyc tagged antibody, V2 will be detected by FLAG tagged antibody.

V1 variant will specifically detected using cMyc (Green channel).

V2 variant will specifically detected using FLAG (Red channel).

### Example 3. c9orf72 RNAi Knockdown

Compared to other technologies, such as nanoparticles or RNA transfection, gene therapy provides precise, efficient and long-term gene expression regulation in vivo. MicroRNA (miRNA) is applied to achieve mutant mRNA transcript down-regulation, after endogenous processing with Drosha cleavage, preserving fidelity and efficiency against target mRNA transcripts. Structure and sequence of the miRNA scaffold is critical for the entire process as documented previously. Efforts are put into investigating, designing, and screening of most appropriate miRNA scaffolds.

To minimize off-target effect, miRNA expression is maintained at its minimum but effective level, and multiple miRNA were explored. The following Tables set forth miRNA-c9orf72 sense and antisense libraries that were constructed to be employed for c9orf72 knockdown.

TABLE 3
miRNA-C9ORF72-ANTIsense-Library

| miR Name- | | | | | | | |
|---|---|---|---|---|---|---|---|
| Append (SEQ ID | | | | | | | |
| NOS 107-146, | | | | | | | |
| respectively, | | | 5′ miR | mature-miR. | | | |
| in order of | | | flanking | Loop sequence (19 nt). | 3′ miR flanking | | |
| appearance) | attB5 | 5′-buffer | region | 21-mer target | region | 3′-buffer | attB2 |
| AntiSense_rAAV- | GGGGACAACTTTGT | TTAAAGGGAGGTAGTG | CTGGAGGCTTGCTG | TTCAGTGTCAGCCTTTCATAC | GACACAAGGCCTGTTACTA | CAGATCTGGCCGCAC | AACCCAGCTTTCTTGT |
| miR_1 | ATACAAAAGTTGTA | AGTCGACCAGTGGATC | AAGGCTGTATGCT | GTTTTGGCCACTGACTGACGT | GCACTCACATGGAACAAAT | TCGAGATATCTAG | ACAAAGTGGTCCCC |
| | | | | ATGAAACTGACACTGAA | GGCC | | |
| AntiSense_rAAV- | GGGGACAACTTTGT | TTAAAGGGAGGTAGTG | CTGGAGGCTTGCTG | ATCAGAAGCACTTTAGTCCTG | GACACAAGGCCTGTTACTA | CAGATCTGGCCGCAC | AACCCAGCTTTCTTGT |
| miR_2 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | GGACTAGTGCTTCTGAT | GGCC | | |
| AntiSense_rAAV- | GGGGACAACTTTGT | TTAAAGGGAGGTAGTG | CTGGAGGCTTGCTG | TTGAATCAGAAGCACTTTAGT | GACACAAGGCCTGTTACTA | CAGATCTGGCCGCAC | AACCCAGCTTTCTTGT |
| miR_3 | ATACAAAAGTTGTA | AGTCGACCAGTGGATC | AAGGCTGTATGCT | GTTTTGGCCACTGACTGACAC | GCACTCACATGGAACAAAT | TCGAGATATCTAG | ACAAAGTGGTCCCC |
| | | | | TAAAGTTTCTGATTCAA | GGCC | | |
| AntiSense_rAAV- | GGGGACAACTTTGT | TTAAAGGGAGGTAGTG | CTGGAGGCTTGCTG | TAACCTAAGAGCCTTAATGGC | GACACAAGGCCTGTTACTA | CAGATCTGGCCGCAC | AACCCAGCTTTCTTGT |
| miR_4 | ATACAAAAGTTGTA | AGTCGACCAGTGGATC | AAGGCTGTATGCT | GTTTTGGCCACTGACTGACGC | GCACTCACATGGAACAAAT | TCGAGATATCTAG | ACAAAGTGGTCCCC |
| | | | | CATTAACTCTTAGGTTA | GGCC | | |
| AntiSense_rAAV- | GGGGACAACTTTGT | TTAAAGGGAGGTAGTG | CTGGAGGCTTGCTG | TCATGATGGAGTATCAGAGGC | GACACAAGGCCTGTTACTA | CAGATCTGGCCGCAC | AACCCAGCTTTCTTGT |
| miR_5 | ATACAAAAGTTGTA | AGTCGACCAGTGGATC | AAGGCTGTATGCT | GTTTTGGCCACTGACTGACGC | GCACTCACATGGAACAAAT | TCGAGATATCTAG | ACAAAGTGGTCCCC |
| | | | | CTCTGACTCCATCATGA | GGCC | | |
| AntiSense_rAAV- | GGGGACAACTTTGT | TTAAAGGGAGGTAGTG | CTGGAGGCTTGCTG | TAATAGTACCTAATGTGTAGG | GACACAAGGCCTGTTACTA | CAGATCTGGCCGCAC | AACCCAGCTTTCTTGT |
| miR_6 | ATACAAAAGTTGTA | AGTCGACCAGTGGATC | AAGGCTGTATGCT | GTTTTGGCCACTGACTGACCC | GCACTCACATGGAACAAAT | TCGAGATATCTAG | ACAAAGTGGTCCCC |
| | | | | TACACAAGGTACTATTA | GGCC | | |
| AntiSense_rAAV- | GGGGACAACTTTGT | TTAAAGGGAGGTAGTG | CTGGAGGCTTGCTG | AAAGCTAACAGAATCCTTTCA | GACACAAGGCCTGTTACTA | CAGATCTGGCCGCAC | AACCCAGCTTTCTTGT |
| miR_7 | ATACAAAAGTTGTA | AGTCGACCAGTGGATC | AAGGCTGTATGCT | GTTTTGGCCACTGACTGACTG | GCACTCACATGGAACAAAT | TCGAGATATCTAG | ACAAAGTGGTCCCC |
| | | | | AAAGGACTGTTAGCTTT | GGCC | | |
| AntiSense_rAAV- | GGGGACAACTTTGT | TTAAAGGGAGGTAGTG | CTGGAGGCTTGCTG | CATTAAAGCTAACAGAATCCT | GACACAAGGCCTGTTACTA | CAGATCTGGCCGCAC | AACCCAGCTTTCTTGT |
| miR_8 | ATACAAAAGTTGTA | AGTCGACCAGTGGATC | AAGGCTGTATGCT | GTTTTGGCCACTGACTGACAG | GCACTCACATGGAACAAAT | TCGAGATATCTAG | ACAAAGTGGTCCCC |
| | | | | GATTCTTAGCTTTAATG | GGCC | | |
| AntiSense_rAAV- | GGGGACAACTTTGT | TTAAAGGGAGGTAGTG | CTGGAGGCTTGCTG | ATAACAGACTGTCTACTTAGA | GACACAAGGCCTGTTACTA | CAGATCTGGCCGCAC | AACCCAGCTTTCTTGT |
| miR_9 | ATACAAAAGTTGTA | AGTCGACCAGTGGATC | AAGGCTGTATGCT | GTTTTGGCCACTGACTGACTC | GCACTCACATGGAACAAAT | TCGAGATATCTAG | ACAAAGTGGTCCCC |
| | | | | TAAGTACAGTCTGTTAT | GGCC | | |
| AntiSense_rAAV- | GGGGACAACTTTGT | TTAAAGGGAGGTAGTG | CTGGAGGCTTGCTG | AATAACAGACTGTCTACTTAG | GACACAAGGCCTGTTACTA | CAGATCTGGCCGCAC | AACCCAGCTTTCTTGT |
| miR_10 | ATACAAAAGTTGTA | AGTCGACCAGTGGATC | AAGGCTGTATGCT | GTTTTGGCCACTGACTGACCT | GCACTCACATGGAACAAAT | TCGAGATATCTAG | ACAAAGTGGTCCCC |
| | | | | AAGTAGAGTCTGTTATT | GGCC | | |
| AntiSense_rAAV- | GGGGACAACTTTGT | TTAAAGGGAGGTAGTG | CTGGAGGCTTGCTG | TGAAGTTTATGGTAGTGCACA | GACACAAGGCCTGTTACTA | CAGATCTGGCCGCAC | AACCCAGCTTTCTTGT |
| miR_11 | ATACAAAAGTTGTA | AGTCGACCAGTGGATC | AAGGCTGTATGCT | GTTTTGGCCACTGACTGACTG | GCACTCACATGGAACAAAT | TCGAGATATCTAG | ACAAAGTGGTCCCC |
| | | | | TGCACTCATAAACTTCA | GGCC | | |
| AntiSense_rAAV- | GGGGACAACTTTGT | TTAAAGGGAGGTAGTG | CTGGAGGCTTGCTG | TTCTTCTGAAGTTTATGGTAG | GACACAAGGCCTGTTACTA | CAGATCTGGCCGCAC | AACCCAGCTTTCTTGT |
| miR_12 | ATACAAAAGTTGTA | AGTCGACCAGTGGATC | AAGGCTGTATGCT | GTTTTGGCCACTGACTGACCT | GCACTCACATGGAACAAAT | TCGAGATATCTAG | ACAAAGTGGTCCCC |
| | | | | ACCATACTTCAGAAGAA | GGCC | | |
| AntiSense_rAAV- | GGGGACAACTTTGT | TTAAAGGGAGGTAGTG | CTGGAGGCTTGCTG | TTAACCTGCTTGACCAGCTTT | GACACAAGGCCTGTTACTA | CAGATCTGGCCGCAC | AACCCAGCTTTCTTGT |
| miR_13 | ATACAAAAGTTGTA | AGTCGACCAGTGGATC | AAGGCTGTATGCT | GTTTTGGCCACTGACTGACAA | GCACTCACATGGAACAAAT | TCGAGATATCTAG | ACAAAGTGGTCCCC |
| | | | | AGCTGGAAGCAGGTTAA | GGCC | | |
| AntiSense_rAAV- | GGGGACAACTTTGT | TTAAAGGGAGGTAGTG | CTGGAGGCTTGCTG | TGTTTAACCTGCTTGACCAGC | GACACAAGGCCTGTTACTA | CAGATCTGGCCGCAC | AACCCAGCTTTCTTGT |
| miR_14 | ATACAAAAGTTGTA | AGTCGACCAGTGGATC | AAGGCTGTATGCT | GTTTTGGCCACTGACTGACGC | GCACTCACATGGAACAAAT | TCGAGATATCTAG | ACAAAGTGGTCCCC |
| | | | | TGGTCACAGGTTAAACA | GGCC | | |
| AntiSense_rAAV- | GGGGACAACTTTGT | TTAAAGGGAGGTAGTG | CTGGAGGCTTGCTG | AAATTGTTTAACCTGCTTGAC | GACACAAGGCCTGTTACTA | CAGATCTGGCCGCAC | AACCCAGCTTTCTTGT |
| miR_15 | ATACAAAAGTTGTA | AGTCGACCAGTGGATC | AAGGCTGTATGCT | GTTTTGGCCACTGACTGACGT | GCACTCACATGGAACAAAT | TCGAGATATCTAG | ACAAAGTGGTCCCC |
| | | | | CAAGCATTAAACAATTT | GGCC | | |
| AntiSense_rAAV- | GGGGACAACTTTGT | TTAAAGGGAGGTAGTG | CTGGAGGCTTGCTG | ATTTAGGTTAGTCTCCTGATT | GACACAAGGCCTGTTACTA | CAGATCTGGCCGCAC | AACCCAGCTTTCTTGT |
| miR_16 | ATACAAAAGTTGTA | AGTCGACCAGTGGATC | AAGGCTGTATGCT | GTTTTGGCCACTGACTGACAA | GCACTCACATGGAACAAAT | TCGAGATATCTAG | ACAAAGTGGTCCCC |
| | | | | TCAGGACTAACCTAAAT | GGCC | | |
| AntiSense_rAAV- | GGGGACAACTTTGT | TTAAAGGGAGGTAGTG | CTGGAGGCTTGCTG | ACCTTTAGGAAACTATTCTTG | GACACAAGGCCTGTTACTA | CAGATCTGGCCGCAC | AACCCAGCTTTCTTGT |
| miR_17 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | AGAATATTCCTAAAGGT | GGCC | | |
| AntiSense_rAAV- | GGGGACAACTTTGT | TTAAAGGGAGGTAGTG | CTGGAGGCTTGCTG | AAGAGATACCTTTAGGAAACT | GACACAAGGCCTGTTACTA | CAGATCTGGCCGCAC | AACCCAGCTTTCTTGT |
| miR_18 | ATACAAAAGTTGTA | AGTCGACCAGTGGATC | AAGGCTGTATGCT | GTTTTGGCCACTGACTGACAG | GCACTCACATGGAACAAAT | TCGAGATATCTAG | ACAAAGTGGTCCCC |
| | | | | TTTCCTAGGTATCTCTT | GGCC | | |
| AntiSense_rAAV- | GGGGACAACTTTGT | TTAAAGGGAGGTAGTG | CTGGAGGCTTGCTG | CAAAGTAGTAACCATTAATGG | GACACAAGGCCTGTTACTA | CAGATCTGGCCGCAC | AACCCAGCTTTCTTGT |
| miR_19 | ATACAAAAGTTGTA | AGTCGACCAGTGGATC | AAGGCTGTATGCT | GTTTTGGCCACTGACTGACCC | GCACTCACATGGAACAAAT | TCGAGATATCTAG | ACAAAGTGGTCCCC |
| | | | | ATTAATTTACTACTTTG | GGCC | | |
| AntiSense_rAAV- | GGGGACAACTTTGT | TTAAAGGGAGGTAGTG | CTGGAGGCTTGCTG | TTCACATACAGTATTAGCCAC | GACACAAGGCCTGTTACTA | CAGATCTGGCCGCAC | AACCCAGCTTTCTTGT |
| miR_20 | ATACAAAAGTTGTA | AGTCGACCAGTGGATC | AAGGCTGTATGCT | GTTTTGGCCACTGACTGACGT | GCACTCACATGGAACAAAT | TCGAGATATCTAG | ACAAAGTGGTCCCC |
| | | | | GGCTAACTGTATGTGAA | GGCC | | |
| AntiSense_rAAV- | GGGGACAACTTTGT | TTAAAGGGAGGTAGTG | CTGGAGGCTTGCTG | TTAAGGTTCGCACACGCTATT | GACACAAGGCCTGTTACTA | CAGATCTGGCCGCAC | AACCCAGCTTTCTTGT |
| miR_21 | ATACAAAAGTTGTA | AGTCGACCAGTGGATC | AAGGCTGTATGCT | GTTTTGGCCACTGACTGACAA | GCACTCACATGGAACAAAT | TCGAGATATCTAG | ACAAAGTGGTCCCC |
| | | | | TAGCGTGCGAACCTTAA | GGCC | | |
| AntiSense_rAAV- | GGGGACAACTTTGT | TTAAAGGGAGGTAGTG | CTGGAGGCTTGCTG | ATTAAGGTTCGCACACGCTAT | GACACAAGGCCTGTTACTA | CAGATCTGGCCGCAC | AACCCAGCTTTCTTGT |
| miR_22 | ATACAAAAGTTGTA | AGTCGACCAGTGGATC | AAGGCTGTATGCT | GTTTTGGCCACTGACTGACAT | GCACTCACATGGAACAAAT | TCGAGATATCTAG | ACAAAGTGGTCCCC |
| | | | | AGCGTGCGAACCTTAAT | GGCC | | |
| AntiSense_rAAV- | GGGGACAACTTTGT | TTAAAGGGAGGTAGTG | CTGGAGGCTTGCTG | TATTAAGGTTCGCACACGCTA | GACACAAGGCCTGTTACTA | CAGATCTGGCCGCAC | AACCCAGCTTTCTTGT |
| miR_23 | ATACAAAAGTTGTA | AGTCGACCAGTGGATC | AAGGCTGTATGCT | GTTTTGGCCACTGACTGACTA | GCACTCACATGGAACAAAT | TCGAGATATCTAG | ACAAAGTGGTCCCC |
| | | | | GCGTGTGAACCTTAATA | GGCC | | |
| AntiSense_rAAV- | GGGGACAACTTTGT | TTAAAGGGAGGTAGTG | CTGGAGGCTTGCTG | AACTCATCCACATATTGCAAC | GACACAAGGCCTGTTACTA | CAGATCTGGCCGCAC | AACCCAGCTTTCTTGT |
| miR_24 | ATACAAAAGTTGTA | AGTCGACCAGTGGATC | AAGGCTGTATGCT | GTTTTGGCCACTGACTGACGT | GCACTCACATGGAACAAAT | TCGAGATATCTAG | ACAAAGTGGTCCCC |
| | | | | TGCAATGTGGATGAGTT | GGCC | | |
| AntiSense_rAAV- | GGGGACAACTTTGT | TTAAAGGGAGGTAGTG | CTGGAGGCTTGCTG | AGTAAGTGGAATCTATACACC | GACACAAGGCCTGTTACTA | CAGATCTGGCCGCAC | AACCCAGCTTTCTTGT |
| miR_25 | ATACAAAAGTTGTA | AGTCGACCAGTGGATC | AAGGCTGTATGCT | GTTTTGGCCACTGACTGACGG | GCACTCACATGGAACAAAT | TCGAGATATCTAG | ACAAAGTGGTCCCC |
| | | | | TGTATATTCCACTTACT | GGCC | | |
| AntiSense_rAAV- | GGGGACAACTTTGT | TTAAAGGGAGGTAGTG | CTGGAGGCTTGCTG | AATGCTACTCATCTGTAGTAA | GACACAAGGCCTGTTACTA | CAGATCTGGCCGCAC | AACCCAGCTTTCTTGT |
| miR_26 | ATACAAAAGTTGTA | AGTCGACCAGTGGATC | AAGGCTGTATGCT | GTTTTGGCCACTGACTGACTT | GCACTCACATGGAACAAAT | TCGAGATATCTAG | ACAAAGTGGTCCCC |
| | | | | ACTACATGAGTAGCATT | GGCC | | |
| AntiSense_rAAV- | GGGGACAACTTTGT | TTAAAGGGAGGTAGTG | CTGGAGGCTTGCTG | TGTAGTAAGTGCCATCTCACA | GACACAAGGCCTGTTACTA | CAGATCTGGCCGCAC | AACCCAGCTTTCTTGT |
| miR_27 | ATACAAAAGTTGTA | AGTCGACCAGTGGATC | AAGGCTGTATGCT | GTTTTGGCCACTGACTGACTG | GCACTCACATGGAACAAAT | TCGAGATATCTAG | ACAAAGTGGTCCCC |
| | | | | TGAGATCACTTACTACA | GGCC | | |
| AntiSense_rAAV- | GGGGACAACTTTGT | TTAAAGGGAGGTAGTG | CTGGAGGCTTGCTG | TACTCACTGTAGTAAGTGCCA | GACACAAGGCCTGTTACTA | CAGATCTGGCCGCAC | AACCCAGCTTTCTTGT |
| miR_28 | ATACAAAAGTTGTA | AGTCGACCAGTGGATC | AAGGCTGTATGCT | GTTTTGGCCACTGACTGACTG | GCACTCACATGGAACAAAT | TCGAGATATCTAG | ACAAAGTGGTCCCC |
| | | | | GCACTTTACAGTGAGTA | GGCC | | |
| AntiSense_rAAV- | GGGGACAACTTTGT | TTAAAGGGAGGTAGTG | CTGGAGGCTTGCTG | AAATGCTACTCACTGTAGTAA | GACACAAGGCCTGTTACTA | CAGATCTGGCCGCAC | AACCCAGCTTTCTTGT |
| miR_29 | ATACAAAAGTTGTA | AGTCGACCAGTGGATC | AAGGCTGTATGCT | GTTTTGGCCACTGACTGACTT | GCACTCACATGGAACAAAT | TCGAGATATCTAG | ACAAAGTGGTCCCC |
| | | | | ACTACAGAGTAGCATTT | GGCC | | |
| AntiSense_rAAV- | GGGGACAACTTTGT | TTAAAGGGAGGTAGTG | CTGGAGGCTTGCTG | TTAAATGCTACTCACTGTAGT | GACACAAGGCCTGTTACTA | CAGATCTGGCCGCAC | AACCCAGCTTTCTTGT |
| miR_30 | ATACAAAAGTTGTA | AGTCGACCAGTGGATC | AAGGCTGTATGCT | GTTTTGGCCACTGACTGACAC | GCACTCACATGGAACAAAT | TCGAGATATCTAG | ACAAAGTGGTCCCC |
| | | | | TACAGTGTAGCATTTAA | GGCC | | |
| AntiSense_rAAV- | GGGGACAACTTTGT | TTAAAGGGAGGTAGTG | CTGGAGGCTTGCTG | AAACTTAGCACTCTACTAACA | GACACAAGGCCTGTTACTA | CAGATCTGGCCGCAC | AACCCAGCTTTCTTGT |
| miR_31 | ATACAAAAGTTGTA | AGTCGACCAGTGGATC | AAGGCTGTATGCT | GTTTTGGCCACTGACTGACTG | GCACTCACATGGAACAAAT | TCGAGATATCTAG | ACAAAGTGGTCCCC |
| | | | | TTAGTAGTGCTAAGTTT | GGCC | | |
| AntiSense_rAAV- | GGGGACAACTTTGT | TTAAAGGGAGGTAGTG | CTGGAGGCTTGCTG | ATACCAATCAGGGAAGAGATG | GACACAAGGCCTGTTACTA | CAGATCTGGCCGCAC | AACCCAGCTTTCTTGT |
| miR_32 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | TCTCTTCTGATTGGTAT | GGCC | | |
| AntiSense_rAAV- | GGGGACAACTTTGT | TTAAAGGGAGGTAGTG | CTGGAGGCTTGCTG | CTAAATACCAATCAGGGAAGA | GACACAAGGCCTGTTACTA | CAGATCTGGCCGCAC | AACCCAGCTTTCTTGT |
| miR_33 | ATACAAAAGTTGTA | AGTCGACCAGTGGATC | AAGGCTGTATGCT | GTTTTGGCCACTGACTGACTC | GCACTCACATGGAACAAAT | TCGAGATATCTAG | ACAAAGTGGTCCCC |
| | | | | TTCCCTTTGGTATTTAG | GGCC | | |
| AntiSense_rAAV- | GGGGACAACTTTGT | TTAAAGGGAGGTAGTG | CTGGAGGCTTGCTG | TAAACAGCATGGTTACAAGTA | GACACAAGGCCTGTTACTA | CAGATCTGGCCGCAC | AACCCAGCTTTCTTGT |
| miR_34 | ATACAAAAGTTGTA | AGTCGACCAGTGGATC | AAGGCTGTATGCT | GTTTTGGCCACTGACTGACTA | GCACTCACATGGAACAAAT | TCGAGATATCTAG | ACAAAGTGGTCCCC |
| | | | | CTTGTACATGCTGTTTA | GGCC | | |
| AntiSense_rAAV- | GGGGACAACTTTGT | TTAAAGGGAGGTAGTG | CTGGAGGCTTGCTG | ATAAACAGCATGGTTACAAGT | GACACAAGGCCTGTTACTA | CAGATCTGGCCGCAC | AACCCAGCTTTCTTGT |
| miR_35 | ATACAAAAGTTGTA | AGTCGACCAGTGGATC | AAGGCTGTATGCT | GTTTTGGCCACTGACTGACAC | GCACTCACATGGAACAAAT | TCGAGATATCTAG | ACAAAGTGGTCCCC |
| | | | | TTGTAAATGCTGTTTAT | GGCC | | |
| AntiSense_rAAV- | GGGGACAACTTTGT | TTAAAGGGAGGTAGTG | CTGGAGGCTTGCTG | TTCTGGTACTGTAAACAGTTC | GACACAAGGCCTGTTACTA | CAGATCTGGCCGCAC | AACCCAGCTTTCTTGT |
| miR_36 | ATACAAAAGTTGTA | AGTCGACCAGTGGATC | AAGGCTGTATGCT | GTTTTGGCCACTGACTGACGA | GCACTCACATGGAACAAAT | TCGAGATATCTAG | ACAAAGTGGTCCCC |
| | | | | ACTGTTCAGTACCAGAA | GGCC | | |
| AntiSense_rAAV- | GGGGACAACTTTGT | TTAAAGGGAGGTAGTG | CTGGAGGCTTGCTG | ATGAACTTCACCTTCCAGTCT | GACACAAGGCCTGTTACTA | CAGATCTGGCCGCAC | AACCCAGCTTTCTTGT |
| miR_37 | ATACAAAAGTTGTA | AGTCGACCAGTGGATC | AAGGCTGTATGCT | GTTTTGGCCACTGACTGACAG | GCACTCACATGGAACAAAT | TCGAGATATCTAG | ACAAAGTGGTCCCC |
| | | | | ACTGGAGTGAAGTTCAT | GGCC | | |
| AntiSense_rAAV- | GGGGACAACTTTGT | TTAAAGGGAGGTAGTG | CTGGAGGCTTGCTG | TTAGATAGTTCCCAGGAGGAC | GACACAAGGCCTGTTACTA | CAGATCTGGCCGCAC | AACCCAGCTTTCTTGT |
| miR_38 | ATACAAAAGTTGTA | AGTCGACCAGTGGATC | AAGGCTGTATGCT | GTTTTGGCCACTGACTGACGT | GCACTCACATGGAACAAAT | TCGAGATATCTAG | ACAAAGTGGTCCCC |
| | | | | CCTCCTGAACTATCTAA | GGCC | | |
| AntiSense_rAAV- | GGGGACAACTTTGT | TTAAAGGGAGGTAGTG | CTGGAGGCTTGCTG | AACAAAGTAAACCAAGGAGGA | GACACAAGGCCTGTTACTA | CAGATCTGGCCGCAC | AACCCAGCTTTCTTGT |
| miR_39 | ATACAAAAGTTGTA | AGTCGACCAGTGGATC | AAGGCTGTATGCT | GTTTTGGCCACTGACTGACTC | GCACTCACATGGAACAAAT | TCGAGATATCTAG | ACAAAGTGGTCCCC |
| | | | | CTCCTTTTTACTTTGTT | GGCC | | |
| AntiSense_rAAV- | GGGGACAACTTTGT | TTAAAGGGAGGTAGTG | CTGGAGGCTTGCTG | AAACAAAGTAAACCAAGGAGG | GACACAAGGCCTGTTACTA | CAGATCTGGCCGCAC | AACCCAGCTTTCTTGT |
| miR_40 | ATACAAAAGTTGTA | AGTCGACCAGTGGATC | AAGGCTGTATGCT | GTTTTGGCCACTGACTGACCC | GCACTCACATGGAACAAAT | TCGAGATATCTAG | ACAAAGTGGTCCCC |
| | | | | TCCTTGTTACTTTGTTT | GGCC | | |

TABLE 4
miARNA-C9ORF72-sense-Library

| miR Name- | | | | | | | |
|---|---|---|---|---|---|---|---|
| Append (SEQ ID | | | | | | | |
| NOS 147-186, | | | | | | | |
| respectively, | | | | mature-miR. | | | |
| in order of | | | 5′ miR | Loop sequence (19 nt) | 3′ miR flanking | | |
| appearance) | attB5 | 5′-buffer | flanking region | 21-mer target | region | 3′-buffer | attB2 |
| Sense_rAAV- | GGGGACAACTTTGT | TTAAAGGGAGGTAGTG | CTGGAGGCTTGCTG | TAGTATGTATGACAAAGTCCT | GACACAAGGCCTGTTACTA | CAGATCTGGCCGCAC | AACCCAGCTTTCTTGT |
| miR_41 | ATACAAAAGTTGTA | AGTCGACCAGTGGATC | AAGGCTGTATGCT | GTTTTGGCCACTGACTGACAG | GCACTCACATGGAACAAAT | TCGAGATATCTAG | ACAAAGTGGTCCCC |
| | | | | GACTTTCATACATACTA | GGCC | | |
| Sense_rAAV- | GGGGACAACTTTGT | TTAAAGGGAGGTAGTG | CTGGAGGCTTGCTG | TTGCTAAAGTGGCTAATACTG | GACACAAGGCCTGTTACTA | CAGATCTGGCCGCAC | AACCCAGCTTTCTTGT |
| miR_42 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | GTATTACACTTTAGCAA | GGCC | | |
| Sense_rAAV- | GGGGACAACTTTGT | TTAAAGGGAGGTAGTG | CTGGAGGCTTGCTG | AAACGTCCTCAACAAATGATT | GACACAAGGCCTGTTACTA | CAGATCTGGCCGCAC | AACCCAGCTTTCTTGT |
| miR_43 | ATACAAAAGTTGTA | AGTCGACCAGTGGATC | AAGGCTGTATGCT | GTTTTGGCCACTGACTGACAA | GCACTCACATGGAACAAAT | TCGAGATATCTAG | ACAAAGTGGTCCCC |
| | | | | TCATTTTGAGGACGTTT | GGCC | | |
| Sense_rAAV- | GGGGACAACTTTGT | TTAAAGGGAGGTAGTG | CTGGAGGCTTGCTG | AGAATCAGGAGACTAACCTAA | GACACAAGGCCTGTTACTA | CAGATCTGGCCGCAC | AACCCAGCTTTCTTGT |
| miR_44 | ATACAAAAGTTGTA | AGTCGACCAGTGGATC | AAGGCTGTATGCT | GTTTTGGCCACTGACTGACTT | GCACTCACATGGAACAAAT | TCGAGATATCTAG | ACAAAGTGGTCCCC |
| | | | | AGGTTACTCCTGATTCT | GGCC | | |
| Sense_rAAV- | GGGGACAACTTTGT | TTAAAGGGAGGTAGTG | CTGGAGGCTTGCTG | TTCATTTCCGAGAATCAAGAC | GACACAAGGCCTGTTACTA | CAGATCTGGCCGCAC | AACCCAGCTTTCTTGT |
| miR_45 | ATACAAAAGTTGTA | AGTCGACCAGTGGATC | AAGGCTGTATGCT | GTTTTGGCCACTGACTGACGT | GCACTCACATGGAACAAAT | TCGAGATATCTAG | ACAAAGTGGTCCCC |
| | | | | CTTGATTCGGAAATGAA | GGCC | | |
| Sense_rAAV- | GGGGACAACTTTGT | TTAAAGGGAGGTAGTG | CTGGAGGCTTGCTG | TAGTCTGGCTGTAACATAGTG | GACACAAGGCCTGTTACTA | CAGATCTGGCCGCAC | AACCCAGCTTTCTTGT |
| miR_46 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | CTATGTCAGCCAGACTA | GGCC | | |
| Sense_rAAV- | GGGGACAACTTTGT | TTAAAGGGAGGTAGTG | CTGGAGGCTTGCTG | TTAGTCTGGCTGTAACATAGT | GACACAAGGCCTGTTACTA | CAGATCTGGCCGCAC | AACCCAGCTTTCTTGT |
| miR_47 | ATACAAAAGTTGTA | AGTCGACCAGTGGATC | AAGGCTGTATGCT | GTTTTGGCCACTGACTGACAC | GCACTCACATGGAACAAAT | TCGAGATATCTAG | ACAAAGTGGTCCCC |
| | | | | TATGTTAGCCAGACTAA | GGCC | | |
| Sense_rAAV- | GGGGACAACTTTGT | TTAAAGGGAGGTAGTG | CTGGAGGCTTGCTG | ATAGGTGAGCATAAGATGGTA | GACACAAGGCCTGTTACTA | CAGATCTGGCCGCAC | AACCCAGCTTTCTTGT |
| miR_48 | ATACAAAAGTTGTA | AGTCGACCAGTGGATC | AAGGCTGTATGCT | GTTTTGGCCACTGACTGACTA | GCACTCACATGGAACAAAT | TCGAGATATCTAG | ACAAAGTGGTCCCC |
| | | | | CCATCTTGCTCACCTAT | GGCC | | |
| Sense_rAAV- | GGGGACAACTTTGT | TTAAAGGGAGGTAGTG | CTGGAGGCTTGCTG | AATCTAAGTAGACAGTCTGTT | GACACAAGGCCTGTTACTA | CAGATCTGGCCGCAC | AACCCAGCTTTCTTGT |
| miR_49 | ATACAAAAGTTGTA | AGTCGACCAGTGGATC | AAGGCTGTATGCT | GTTTTGGCCACTGACTGACAA | GCACTCACATGGAACAAAT | TCGAGATATCTAG | ACAAAGTGGTCCCC |
| | | | | CAGACTCTACTTAGATT | GGCC | | |
| Sense_rAAV- | GGGGACAACTTTGT | TTAAAGGGAGGTAGTG | CTGGAGGCTTGCTG | AGAACAATCTAAGTAGACAGT | GACACAAGGCCTGTTACTA | CAGATCTGGCCGCAC | AACCCAGCTTTCTTGT |
| miR_50 | ATACAAAAGTTGTA | AGTCGACCAGTGGATC | AAGGCTGTATGCT | GTTTTGGCCACTGACTGACAC | GCACTCACATGGAACAAAT | TCGAGATATCTAG | ACAAAGTGGTCCCC |
| | | | | TGTCTATAGATTGTTCT | GGCC | | |
| Sense_rAAV- | GGGGACAACTTTGT | TTAAAGGGAGGTAGTG | CTGGAGGCTTGCTG | TTAAGTACTAAACTCCACTGC | GACACAAGGCCTGTTACTA | CAGATCTGGCCGCAC | AACCCAGCTTTCTTGT |
| miR_51 | ATACAAAAGTTGTA | AGTCGACCAGTGGATC | AAGGCTGTATGCT | GTTTTGGCCACTGACTGACGC | GCACTCACATGGAACAAAT | TCGAGATATCTAG | ACAAAGTGGTCCCC |
| | | | | AGTGGATTAGTACTTAA | GGCC | | |
| Sense_rAAV- | GGGGACAACTTTGT | TTAAAGGGAGGTAGTG | CTGGAGGCTTGCTG | AACTCTTAAGTACTAAACTCC | GACACAAGGCCTGTTACTA | CAGATCTGGCCGCAC | AACCCAGCTTTCTTGT |
| miR_52 | ATACAAAAGTTGTA | AGTCGACCAGTGGATC | AAGGCTGTATGCT | GTTTTGGCCACTGACTGACGG | GCACTCACATGGAACAAAT | TCGAGATATCTAG | ACAAAGTGGTCCCC |
| | | | | AGTTTAACTTAAGAGTT | GGCC | | |
| Sense_rAAV- | GGGGACAACTTTGT | TTAAAGGGAGGTAGTG | CTGGAGGCTTGCTG | AATTCAGGCACCTTGCCCACG | GACACAAGGCCTGTTACTA | CAGATCTGGCCGCAC | AACCCAGCTTTCTTGT |
| miR_53 | ATACAAAAGTTGTA | AGTCGACCAGTGGATC | AAGGCTGTATGCT | GTTTTGGCCACTGACTGACCG | GCACTCACATGGAACAAAT | TCGAGATATCTAG | ACAAAGTGGTCCCC |
| | | | | TGGGCAGTGCCTGAATT | GGCC | | |
| Sense_rAAV- | GGGGACAACTTTGT | TTAAAGGGAGGTAGTG | CTGGAGGCTTGCTG | AGAGAATTCAGGCACCTTGCC | GACACAAGGCCTGTTACTA | CAGATCTGGCCGCAC | AACCCAGCTTTCTTGT |
| miR_54 | ATACAAAAGTTGTA | AGTCGACCAGTGGATC | AAGGCTGTATGCT | GTTTTGGCCACTGACTGACGG | GCACTCACATGGAACAAAT | TCGAGATATCTAG | ACAAAGTGGTCCCC |
| | | | | CAAGGTCTGAATTCTCT | GGCC | | |
| Sense_rAAV- | GGGGACAACTTTGT | TTAAAGGGAGGTAGTG | CTGGAGGCTTGCTG | ATAACAACCCTACACATTAGG | GACACAAGGCCTGTTACTA | CAGATCTGGCCGCAC | AACCCAGCTTTCTTGT |
| miR_55 | ATACAAAAGTTGTA | AGTCGACCAGTGGATC | AAGGCTGTATGCT | GTTTTGGCCACTGACTGACCC | GCACTCACATGGAACAAAT | TCGAGATATCTAG | ACAAAGTGGTCCCC |
| | | | | TAATGTAGGGTTGTTAT | GGCC | | |
| Sense_rAAV- | GGGGACAACTTTGT | TTAAAGGGAGGTAGTG | CTGGAGGCTTGCTG | TTCTGATTCAAGCCATTAAGG | GACACAAGGCCTGTTACTA | CAGATCTGGCCGCAC | AACCCAGCTTTCTTGT |
| miR_56 | ATACAAAAGTTGTA | AGTCGACCAGTGGATC | AAGGCTGTATGCT | GTTTTGGCCACTGACTGACCC | GCACTCACATGGAACAAAT | TCGAGATATCTAG | ACAAAGTGGTCCCC |
| | | | | TTAATGTTGAATCAGAA | GGCC | | |
| Sense_rAAV- | GGGGACAACTTTGT | TTAAAGGGAGGTAGTG | CTGGAGGCTTGCTG | TACAGGACTAAAGTGCTTCTG | GACACAAGGCCTGTTACTA | CAGATCTGGCCGCAC | AACCCAGCTTTCTTGT |
| miR_57 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | GAAGCATTAGTCCTGTA | GGCC | | |
| Sense_rAAV- | GGGGACAACTTTGT | TTAAAGGGAGGTAGTG | CTGGAGGCTTGCTG | AACAGATACAGGACTAAAGTG | GACACAAGGCCTGTTACTA | CAGATCTGGCCGCAC | AACCCAGCTTTCTTGT |
| miR_58 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | CTTTAGCTGTATCTGTT | GGCC | | |
| Sense_rAAV- | GGGGACAACTTTGT | TTAAAGGGAGGTAGTG | CTGGAGGCTTGCTG | ATGAAAGGCTGACACTGAACA | GACACAAGGCCTGTTACTA | CAGATCTGGCCGCAC | AACCCAGCTTTCTTGT |
| miR_59 | ATACAAAAGTTGTA | AGTCGACCAGTGGATC | AAGGCTGTATGCT | GTTTTGGCCACTGACTGACTG | GCACTCACATGGAACAAAT | TCGAGATATCTAG | ACAAAGTGGTCCCC |
| | | | | TTCAGTCAGCCTTTCAT | GGCC | | |
| Sense_rAAV- | GGGGACAACTTTGT | TTAAAGGGAGGTAGTG | CTGGAGGCTTGCTG | AATGATGTATGAAAGGCTGAC | GACACAAGGCCTGTTACTA | CAGATCTGGCCGCAC | AACCCAGCTTTCTTGT |
| miR_60 | ATACAAAAGTTGTA | AGTCGACCAGTGGATC | AAGGCTGTATGCT | GTTTTGGCCACTGACTGACGT | GCACTCACATGGAACAAAT | TCGAGATATCTAG | ACAAAGTGGTCCCC |
| | | | | CAGCCTCATACATCATT | GGCC | | |
| Sense_rAAV- | GGGGACAACTTTGT | TTAAAGGGAGGTAGTG | CTGGAGGCTTGCTG | TGAGATGGCACTTACTACAGT | GACACAAGGCCTGTTACTA | CAGATCTGGCCGCAC | AACCCAGCTTTCTTGT |
| miR_61 | ATACAAAAGTTGTA | AGTCGACCAGTGGATC | AAGGCTGTATGCT | GTTTTGGCCACTGACTGACAC | GCACTCACATGGAACAAAT | TCGAGATATCTAG | ACAAAGTGGTCCCC |
| | | | | TGTAGTGTGCCATCTCA | GGCC | | |
| Sense_rAAV- | GGGGACAACTTTGT | TTAAAGGGAGGTAGTG | CTGGAGGCTTGCTG | ATGAGTAGCATTTACACCACT | GACACAAGGCCTGTTACTA | CAGATCTGGCCGCAC | AACCCAGCTTTCTTGT |
| miR_62 | ATACAAAAGTTGTA | AGTCGACCAGTGGATC | AAGGCTGTATGCT | GTTTTGGCCACTGACTGACAG | GCACTCACATGGAACAAAT | TCGAGATATCTAG | ACAAAGTGGTCCCC |
| | | | | TGGTGTATGCTACTCAT | GGCC | | |
| Sense_rAAV- | GGGGACAACTTTGT | TTAAAGGGAGGTAGTG | CTGGAGGCTTGCTG | TATAGATTCCACTTACTACAG | GACACAAGGCCTGTTACTA | CAGATCTGGCCGCAC | AACCCAGCTTTCTTGT |
| miR_63 | ATACAAAAGTTGTA | AGTCGACCAGTGGATC | AAGGCTGTATGCT | GTTTTGGCCACTGACTGACCT | GCACTCACATGGAACAAAT | TCGAGATATCTAG | ACAAAGTGGTCCCC |
| | | | | GTAGTATGGAATCTATA | GGCC | | |
| Sense_rAAV- | GGGGACAACTTTGT | TTAAAGGGAGGTAGTG | CTGGAGGCTTGCTG | AAACGTACCATTCTGTTTGAT | GACACAAGGCCTGTTACTA | CAGATCTGGCCGCAC | AACCCAGCTTTCTTGT |
| miR_64 | ATACAAAAGTTGTA | AGTCGACCAGTGGATC | AAGGCTGTATGCT | GTTTTGGCCACTGACTGACAT | GCACTCACATGGAACAAAT | TCGAGATATCTAG | ACAAAGTGGTCCCC |
| | | | | CAAACAATGGTACGTTT | GGCC | | |
| Sense_rAAV- | GGGGACAACTTTGT | TTAAAGGGAGGTAGTG | CTGGAGGCTTGCTG | TTTACCGTAAGACACTGTTAA | GACACAAGGCCTGTTACTA | CAGATCTGGCCGCAC | AACCCAGCTTTCTTGT |
| miR_65 | ATACAAAAGTTGTA | AGTCGACCAGTGGATC | AAGGCTGTATGCT | GTTTTGGCCACTGACTGACTT | GCACTCACATGGAACAAAT | TCGAGATATCTAG | ACAAAGTGGTCCCC |
| | | | | AACAGTCTTACGGTAAA | GGCC | | |
| Sense_rAAV- | GGGGACAACTTTGT | TTAAAGGGAGGTAGTG | CTGGAGGCTTGCTG | AATAGCGTGTGCGAACCTTAA | GACACAAGGCCTGTTACTA | CAGATCTGGCCGCAC | AACCCAGCTTTCTTGT |
| miR_66 | ATACAAAAGTTGTA | AGTCGACCAGTGGATC | AAGGCTGTATGCT | GTTTTGGCCACTGACTGACTT | GCACTCACATGGAACAAAT | TCGAGATATCTAG | ACAAAGTGGTCCCC |
| | | | | AAGGTTCACACGCTATT | GGCC | | |
| Sense_rAAV- | GGGGACAACTTTGT | TTAAAGGGAGGTAGTG | CTGGAGGCTTGCTG | TTAAGACCCGCTCTGGAGGAG | GACACAAGGCCTGTTACTA | CAGATCTGGCCGCAC | AACCCAGCTTTCTTGT |
| miR_67 | ATACAAAAGTTGTA | AGTCGACCAGTGGATC | AAGGCTGTATGCT | GTTTTGGCCACTGACTGACCT | GCACTCACATGGAACAAAT | TCGAGATATCTAG | ACAAAGTGGTCCCC |
| | | | | CCTCCAGCGGGTCTTAA | GGCC | | |
| Sense_rAAV- | GGGGACAACTTTGT | TTAAAGGGAGGTAGTG | CTGGAGGCTTGCTG | TTTATCTTAAGACCCGCTCTG | GACACAAGGCCTGTTACTA | CAGATCTGGCCGCAC | AACCCAGCTTTCTTGT |
| miR_68 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | GAGCGGCTTAAGATAAA | GGCC | | |
| Sense_rAAV- | GGGGACAACTTTGT | TTAAAGGGAGGTAGTG | CTGGAGGCTTGCTG | TTTCTCACGAGGCTAGCGAAA | GACACAAGGCCTGTTACTA | CAGATCTGGCCGCAC | AACCCAGCTTTCTTGT |
| miR_69 | ATACAAAAGTTGTA | AGTCGACCAGTGGATC | AAGGCTGTATGCT | GTTTTGGCCACTGACTGACTT | GCACTCACATGGAACAAAT | TCGAGATATCTAG | ACAAAGTGGTCCCC |
| | | | | TCGCTACTCGTGAGAAA | GGCC | | |
| Sense_rAAV- | GGGGACAACTTTGT | TTAAAGGGAGGTAGTG | CTGGAGGCTTGCTG | TTCCAGAGCTTGCTACAGGCT | GACACAAGGCCTGTTACTA | CAGATCTGGCCGCAC | AACCCAGCTTTCTTGT |
| miR_70 | ATACAAAAGTTGTA | AGTCGACCAGTGGATC | AAGGCTGTATGCT | GTTTTGGCCACTGACTGACAG | GCACTCACATGGAACAAAT | TCGAGATATCTAG | ACAAAGTGGTCCCC |
| | | | | CCTGTAAAGCTCTGGAA | GGCC | | |
| Sense_rAAV- | GGGGACAACTTTGT | TTAAAGGGAGGTAGTG | CTGGAGGCTTGCTG | TGTACTATCAGCATGTAGCAG | GACACAAGGCCTGTTACTA | CAGATCTGGCCGCAC | AACCCAGCTTTCTTGT |
| miR_71 | ATACAAAAGTTGTA | AGTCGACCAGTGGATC | AAGGCTGTATGCT | GTTTTGGCCACTGACTGACCT | GCACTCACATGGAACAAAT | TCGAGATATCTAG | ACAAAGTGGTCCCC |
| | | | | GCTACACTGATAGTACA | GGCC | | |
| Sense_rAAV- | GGGGACAACTTTGT | TTAAAGGGAGGTAGTG | CTGGAGGCTTGCTG | TTCAGATGTACTATCAGCATG | GACACAAGGCCTGTTACTA | CAGATCTGGCCGCAC | AACCCAGCTTTCTTGT |
| miR_72 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | TGCTGAGTACATCTGAA | GGCC | | |
| Sense_rAAV- | GGGGACAACTTTGT | TTAAAGGGAGGTAGTG | CTGGAGGCTTGCTG | AATTAACGTAGAATAGAACCC | GACACAAGGCCTGTTACTA | CAGATCTGGCCGCAC | AACCCAGCTTTCTTGT |
| miR_73 | ATACAAAAGTTGTA | AGTCGACCAGTGGATC | AAGGCTGTATGCT | GTTTTGGCCACTGACTGACGG | GCACTCACATGGAACAAAT | TCGAGATATCTAG | ACAAAGTGGTCCCC |
| | | | | GTTCTACTACGTTAATT | GGCC | | |
| Sense_rAAV- | GGGGACAACTTTGT | TTAAAGGGAGGTAGTG | CTGGAGGCTTGCTG | TAAACCGTCCACTTTCCACAA | GACACAAGGCCTGTTACTA | CAGATCTGGCCGCAC | AACCCAGCTTTCTTGT |
| miR_74 | ATACAAAAGTTGTA | AGTCGACCAGTGGATC | AAGGCTGTATGCT | GTTTTGGCCACTGACTGACTT | GCACTCACATGGAACAAAT | TCGAGATATCTAG | ACAAAGTGGTCCCC |
| | | | | GTGGAATGGACGGTTTA | GGCC | | |
| Sense_rAAV- | GGGGACAACTTTGT | TTAAAGGGAGGTAGTG | CTGGAGGCTTGCTG | TGCACTGGCAGGATCATAGCT | GACACAAGGCCTGTTACTA | CAGATCTGGCCGCAC | AACCCAGCTTTCTTGT |
| miR_75 | ATACAAAAGTTGTA | AGTCGACCAGTGGATC | AAGGCTGTATGCT | GTTTTGGCCACTGACTGACAG | GCACTCACATGGAACAAAT | TCGAGATATCTAG | ACAAAGTGGTCCCC |
| | | | | CTATGACTGCCAGTGCA | GGCC | | |
| Sense_rAAV- | GGGGACAACTTTGT | TTAAAGGGAGGTAGTG | CTGGAGGCTTGCTG | AGAGGTTTCCCAATACACTTT | GACACAAGGCCTGTTACTA | CAGATCTGGCCGCAC | AACCCAGCTTTCTTGT |
| miR_76 | ATACAAAAGTTGTA | AGTCGACCAGTGGATC | AAGGCTGTATGCT | GTTTTGGCCACTGACTGACAA | GCACTCACATGGAACAAAT | TCGAGATATCTAG | ACAAAGTGGTCCCC |
| | | | | AGTGTAGGGAAACCTCT | GGCC | | |
| Sense_rAAV- | GGGGACAACTTTGT | TTAAAGGGAGGTAGTG | CTGGAGGCTTGCTG | TTCAAATTGAGTGAGACGGTG | GACACAAGGCCTGTTACTA | CAGATCTGGCCGCAC | AACCCAGCTTTCTTGT |
| miR_77 | ATACAAAAGTTGTA | AGTCGACCAGTGGATC | AAGGCTGTATGCT | GTTTTGGCCACTGACTGACCA | GCACTCACATGGAACAAAT | TCGAGATATCTAG | ACAAAGTGGTCCCC |
| | | | | CCGTCTCTCAATTTGAA | GGCC | | |
| Sense_rAAV- | GGGGACAACTTTGT | TTAAAGGGAGGTAGTG | CTGGAGGCTTGCTG | CCAAGATTCAAATTGAGTGAG | GACACAAGGCCTGTTACTA | CAGATCTGGCCGCAC | AACCCAGCTTTCTTGT |
| miR_78 | ATACAAAAGTTGTA | AGTCGACCAGTGGATC | AAGGCTGTATGCT | GTTTTGGCCACTGACTGACCT | GCACTCACATGGAACAAAT | TCGAGATATCTAG | ACAAAGTGGTCCCC |
| | | | | CACTCATTGAATCTTGG | GGCC | | |
| Sense_rAAV- | GGGGACAACTTTGT | TTAAAGGGAGGTAGTG | CTGGAGGCTTGCTG | AATACTTGAAGTCATCGTCTT | GACACAAGGCCTGTTACTA | CAGATCTGGCCGCAC | AACCCAGCTTTCTTGT |
| miR_79 | ATACAAAAGTTGTA | AGTCGACCAGTGGATC | AAGGCTGTATGCT | GTTTTGGCCACTGACTGACAA | GCACTCACATGGAACAAAT | TCGAGATATCTAG | ACAAAGTGGTCCCC |
| | | | | GACGATCTTCAAGTATT | GGCC | | |
| Sense_rAAV- | GGGGACAACTTTGT | TTAAAGGGAGGTAGTG | CTGGAGGCTTGCTG | TGAAATGGTAATGACACTACT | GACACAAGGCCTGTTACTA | CAGATCTGGCCGCAC | AACCCAGCTTTCTTGT |
| miR_80 | ATACAAAAGTTGTA | AGTCGACCAGTGGATC | AAGGCTGTATGCT | GTTTTGGCCACTGACTGACAG | GCACTCACATGGAACAAAT | TCGAGATATCTAG | ACAAAGTGGTCCCC |
| | | | | TAGTGTTTACCATTTCA | GGCC | | |

The following miRNA constructs were prepared:

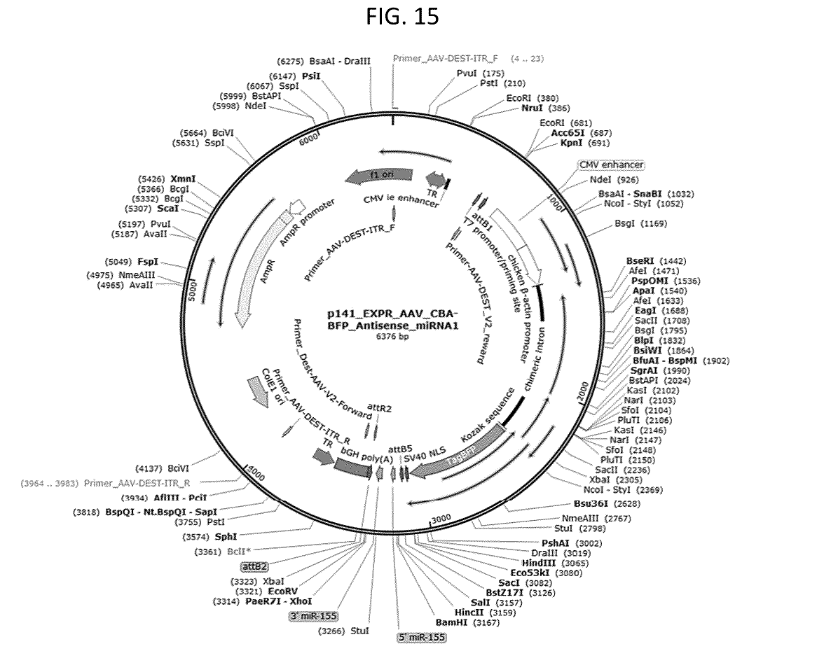

(1) p141_EXPR_AAV_CBA-BFP_Antisense_miRNA1. This construct comprises CBA promoter, BFP sequence, miRNA1 targeting antisense C9orf72, bGH polyA signal. Ampicillin resistance gene. The vector map is shown in FIG. 15. According to some embodiments, the nucleic acid sequence of p141_EXPR_AAV_CBA-BFP_Antisense_miRNA1 comprises SEQ ID NO: 74. According to some embodiments, the nucleic acid sequence of p141_EXPR_AAV_CBA-BFP_Antisense_miRNA1 is at least 85%, at least 90%, at least 95%, at least 96%, at least 97%, at least 98%, at least 99% identical to SEQ ID NO: 74, shown below.

ccggcgaacgtggcgagaaaggaagggaagaaagcgaaaggagcgggcgc
tagggcgctggcaagtgtagcggtcacgctgcgcgtaaccaccacacccg
ccgcgcttaatgcgccgctacagggcgcgtcgcgccattcgccattcagg
ctacgcaactgttgggaagggcgatcggtgcgggcctcttcgctattacg
ccaggctgcaggggggggggggggggggttggccactccctctctgcgcg
ctcgctcgctcactgaggccgggcgaccaaaggtcgcccgacgcccgggc
tttgcccgggcggcctcagtgagcgagcgagcgcgcagagagggagtggc
caactccatcactaggggttcctagatctgaattcgcgacggatcgggag
atctcccgatcccctatggtgcactctcagtacaatctgctctgatgccg
catagttaagccagtatctgctccctgcttgtgtgttggaggtcgctgag
tagtgcgcgagcaaaatttaagctacaacaaggcaaggcttgaccgacaa
ttctctggctaactagagaacccactgcttactggcttatcgaaattaat
acgactcactatagggagacccaagctggctagttaagctatcaacaagt
ttGTACAAAAAAGCAGGCTTACTCAGATCTGAATTCGGTACCTAGTTATT
AATAGTAATCAATTACGGGGTCATTAGTTCATAGCCCATATATGGAGTTC
CGCGTTACATAACTTACGGTAAATGGCCCGCCTGGCTGACCGCCCAACGA
CCCCCGCCCATTGACGTCAATAATGACGTATGTTCCCATAGTAACGCCAA
TAGGGACTTTCCATTGACGTCAATGGGTGGAGTATTTACGGTAAACTGCC
CACTTGGCAGTACATCAAGTGTATCATATGCCAAGTACGCCCCCTATTGA
CGTCAATGACGGTAAATGGCCCGCCTGGCATTATGCCCAGTACATGACCT
TATGGGACTTTCCTACTTGGCAGTACATCTACGTATTAGTCATCGCTATT
ACCATGGTCGAGGTGAGCCCCACGTTCTGCTTCACTCTCCCCATCTCCCC
CCCCTCCCCACCCCCAATTTTGTATTTATTTATTTTTTAATTATTTTGTG
CAGCGATGGGGGCGGGGGGGGGGGGGGGGCGCGCGCCAGGCGGGGCGGGG
CGGGGCGAGGGGCGGGGCGGGGCGAGGCGGAGAGGTGCGGCGGCAGCCAA
TCAGAGCGGCGCGCTCCGAAAGTTTCCTTTTATGGCGAGGCGGCGGCGGC
GGCGGCCCTATAAAAAGCGAAGCGCGCGGCGGGCGGGAGTCGCTGCGCGC
TGCCTTCGCCCCGTGCCCCGCTCCGCCGCCGCCTCGCGCCGCCCGCCCCG
GCTCTGACTGACCGCGTTACTCCCACAGGTGAGCGGGCGGGACGGCCCTT
CTCCTCCGGGCTGTAATTAGCGCTTGGTTTAATGACGGCTTGTTTCTTTT
CTGTGGCTGCGTGAAAGCCTTGAGGGGCTCCGGGAGGGCCCTTTGTGCGG
GGGGAGCGGCTCGGGGGGTGCGTGCGTGTGTGTGTGCGTGGGGAGCGCCG
CGTGCGGCTCCGCGCTGCCCGGCGGCTGTGAGCGCTGCGGGCGCGGCGCG
GGGCTTTGTGCGCTCCGCAGTGTGCGCGAGGGGAGCGCGGCCGGGGGCGG
TGCCCCGCGGTGCGGGGGGGGCTGCGAGGGGAACAAAGGCTGCGTGCGGG
GTGTGTGCGTGGGGGGGTGAGCAGGGGGTGTGGGCGCGTCGGTCGGGCTG
CAACCCCCCCTGCACCCCCCTCCCCGAGTTGCTGAGCACGGCCCGGCTTC
GGGTGCGGGGCTCCGTACGGGGCGTGGCGCGGGGCTCGCCGTGCCGGGCG
GGGGGTGGCGGCAGGTGGGGGTGCCGGGCGGGGCGGGGCCGCCTCGGGCC
GGGGAGGGCTCGGGGGAGGGGCGCGGCGGCCCCCGGAGCGCCGGCGGCTG
TCGAGGCGCGGCGAGCCGCAGCCATTGCCTTTTATGGTAATCGTGCGAGA
GGGCGCAGGGACTTCCTTTGTCCCAAATCTGTGCGGAGCCGAAATCTGGG
AGGCGCCGCCGCACCCCCTCTAGCGGGCGCGGGGCGAAGCGGTGCGGCGC
CGGCAGGAAGGAAATGGGCGGGGAGGGCCTTCGTGCGTCGCCGCGCCGCC
GTCCCCTTCTCCCTCTCCAGCCTCGGGGCTGTCCGCGGGGGGACGGCTGC
CTTCGGGGGGGACGGGGCAGGGCGGGGTTCGGCTTCTGGCGTGTGACCGG
CGGCTCTAGAGCCTCTGCTAACCATGTTCATGCCTTCTTCTTTTTCCTAC
AGCTCCTGGGCAACGCCACCATGGATGAGCGAGCTGATTAAGGAGAACAT
GCACATGAAGCTGTACATGGAGGGCACCGTGGACAACCATCACTTCAAGT
GCACATCCGAGGGCGAAGGCAAGCCCTACGAGGGCACCCAGACCATGAGA
ATCAAGGTGGTCGAGGGCGGCCCTCTCCCCTTCGCCTTCGACATCCTGGC
TACTAGCTTCCTCTACGGCAGCAAGACCTTCATCAACCACACCCAGGGCA
TCCCCGACTTCTTCAAGCAGTCCTTCCCTGAGGGCTTCACATGGGAGAGA
GTCACCACATACGAAGACGGGGGCGTGCTGACCGCTACCCAGGACACCAG
CCTCCAGGACGGCTGCCTCATCTACAACGTCAAGATCAGAGGGGTGAACT
TCACATCCAACGGCCCTGTGATGCAGAAGAAAACACTCGGCTGGGAGGCC
TTCACCGAGACGCTGTACCCCGCTGACGGCGGCCTGGAAGGCAGAAACGA
CATGGCCCTGAAGCTCGTGGGCGGGAGCCATCTGATCGCAAACATCAAGA
CCACATATAGATCCAAGAAACCCGCTAAGAACCTCAAGATGCCTGGCGTC
TACTATGTGGACTACAGACTGGAAAGAATCAAGGAGGCCAACAACGAGAC
CTACGTCGAGCAGCACGAGGTGGCAGTGGCCAGATACTGCGACCTCCCTA
GCAAACTGGGGCACAAGCTTAATGAGGGAGCTCCAAAGAAGAAGCGTAAG
GTAGGTAGTTCCTAGACAACTTTGTATACAAAAGTTGTATTAAAGGGAGG
TAGTGAGTCGACCAGTGGATCCTGGAGGCTTGCTGAAGGCTGTATGCTTT
CAGTGTCAGCCTTTCATACGTTTTGGCCACTGACTGACGTATGAAACTGA
CACTGAAGACACAAGGCCTGTTACTAGCACTCACATGGAACAAATGGCCC
AGATCTGGCCGCACTCGAGATATCTAGAACCCAGCTTTcttgtacaaagt
ggttgatcgctgatcagcctcgactgtgccttctagttgccagccatctg
ttgtttgcccctcccccgtgccttccttgaccctggaaggtgccactccc
actgtcctttcctaataaaatgaggaaattgcatcgcattgtctgagtag
gtgtcattctattctggggggtggggtggggcaggacagcaagggggagg
attgggaagacaatagcaggcatgctggggagagatctaggaacccctag
tgatggagttggccactccctctctgcgcgctcgctcgctcactgaggcc
gcccgggcaaagcccgggcgtcgggcgacctttggtcgcccggcctcagt
gagcgagcgagcgcgcagagagggagtggccaaccccccccccccccccc
ctgcagccctgcattaatgaatcggccaacgcgcggggagaggcggtttg
cgtattgggcgctcttccgcttcctcgctcactgactcgctgcgctcggt
cgttcggctgcggcgagcggtatcagctcactcaaaggcggtaatacggt
tatccacagaatcaggggataacgcaggaaagaacatgtgagcaaaaggc
cagcaaaaggccaggaaccgtaaaaaggccgcgttgctggcgtttttcca
taggctccgcccccctgacgagcatcacaaaaatcgacgctcaagtcaga
ggtggcgaaacccgacaggactataaagataccaggcgtttccccctgga
agctccctcgtgcgctctcctgttccgaccctgccgcttaccggatacct
gtccgcctttctcccttcgggaagcgtggcgctttctcaatgctcacgct
gtaggtatctcagttcggtgtaggtcgttcgctccaagctgggctgtgtg
cacgaaccccccgttcagcccgaccgctgcgccttatccggtaactatcg
tcttgagtccaacccggtaagacacgacttatcgccactggcagcagcca
ctggtaacaggattagcagagcgaggtatgtaggcggtgctacagagttc
ttgaagtggtggcctaactacggctacactagaaggacagtatttggtat
ctgcgctctgctgaagccagttaccttcggaaaaagagttggtagctctt
gatccggcaaacaaaccaccgctggtagcggtggtttttttgtttgcaag
cagcagattacgcgcagaaaaaaaggatctcaagaagatcctttgatctt
ttctacggggtctgacgctcagtggaacgaaaactcacgttaagggattt
tggtcatgagattatcaaaaaggatcttcacctagatccttttaaattaa
aaatgaagttttaaatcaatctaaagtatatatgagtaaacttggtctga
cagttaccaatgcttaatcagtgaggcacctatctcagcgatctgtctat
ttcgttcatccatagttgcctgactccccgtcgtgtagataactacgata
cgggagggcttaccatctggccccagtgctgcaatgataccgcgagaccc
acgctcaccggctccagatttatcagcaataaaccagccagccggaaggg
ccgagcgcagaagtggtcctgcaactttatccgcctccatccagtctatt
aattgttgccgggaagctagagtaagtagttcgccagttaatagtttgcg
caacgttgttgccattgctacaggcatcgtggtgtcacgctcgtcgtttg
gtatggcttcattcagctccggttcccaacgatcaaggcgagttacatga
tcccccatgttgtgcaaaaaagcggttagctccttcggtcctccgatcgt
tgtcagaagtaagttggccgcagtgttatcactcatggttatggcagcac
tgcataattctcttactgtcatgccatccgtaagatgcttttctgtgact
ggtgagtactcaaccaagtcattctgagaatagtgtatgcggcgaccgag
ttgctcttgcccggcgtcaatacgggataataccgcgccacatagcagaa
ctttaaaagtgctcatcattggaaaacgttcttcggggcgaaaactctca
aggatcttaccgctgttgagatccagttcgatgtaacccactcgtgcacc
caactgatcttcagcatcttttactttcaccagcgtttctgggtgagcaa
aaacaggaaggcaaaatgccgcaaaaaagggaataagggcgacacggaaa
tgttgaatactcatactcttcctttttcaatattattgaagcatttatca
gggttattgtctcatgagcggatacatatttgaatgtatttagaaaaata
aacaaataggggttccgcgcacatttccccgaaaagtgccacctgacgtc
taagaaaccattattatcatgacattaacctataaaaataggcgtatcac
gaggccctttcgtctcgcgcgtttcggtgatgacggtgaaaacctctgac
acatgcagctcccggagacggtcacagcttgtctgtaagcggatgccggg
agcagacaagcccgtcagggcgcgtcagcgggtgttggcgggtgtcgggg
ctggcttaactatgcggcatcagagcagattgtactgagagtgcaccata
tgcggtgtgaaataccgcacagatgcgtaaggagaaaataccgcatcagg
aaattgtaaacgttaatattttgttaaaattcgcgttaaatttttgttaa
atcagctcattttttaaccaataggccgaaatcggcaaaatcccttataa
atcaaaagaatagaccgagatagggttgagtgttgttccagtttggaaca
agagtccactattaaagaacgtggactccaacgtcaaagggcgaaaaacc
gtctatcagggcgatggcccactacgtgaaccatcaccctaatcaagttt
tttggggtcgaggtgccgtaaagcactaaatcggaaccctaaagggagcc
cccgatttagagcttgacggggaaag

According to some embodiments, p141_EXPR_AAV_CBA-BFP_Antisense_miRNA1_11-ATTB1 (870 bp) comprises SEQ ID NO: 75, shown below.

NNNNNNNNNNNNNNATCGNNNNNAGNTATTAATAGTAATCAATTACGGGG
TCATTAGTTCATAGCCCATATATGGAGTTCCNCGTTACATAACTTACGGT
AAATGGCCCGCCTGGCTGACCGCCCAACGACCCCCGCCCATTGACGTCAA
TAATGACGTATGTTCCCATAGTAACGCCAATAGGGACTTTCCATTGACGT
CAATGGGTGGAGTATTTACGGTAAACTGCCCACTTGGCAGTACATCAAGT
GTATCATATGCCAAGTACGCCCCCTATTGACGTCAATGACGGTAAATGGC
CCGCCTGGCATTATGCCCAGTACATGACCTTATGGGACTTTCCTACTTGG
CAGTACATCTACGTATTAGTCATCGCTATTACCATGGTCGAGGTGAGCCC
CACGTTCTGCTTCACTCTCCCCATCTCCCCCCCCTCCCCACCCCCAATTT
TGTATTTATTTATTTTTTAATTATTTTGTGCAGCGATGGGGGCGGGGGGG
GGGGGGGGGCGCGCGCCAGGCGGGGCGGGGCGGGGCGAGGGGCGGGGCGG
GGCGAGGCGAAAAGGTGCGGCGGCAGCCAATCAGAGCGGCGCGCTCCGAA
AGTTTCCTTTTATGGCGAGGCGGCGGCGGCGGCGGCCCTATAAAAAGCNA
AGCGCGCGGCGGGCGGGAGTCGCTGCNCGCTGCCTTCGCCCCGTGCCCCG
CTCCGCCGCCGCCTCGCGCCGCCCGCCCCGGCTCTGACTGACCGCGTTAC
TCCCACAGGTGAGCGGGCGGNNNGGCCCTNCTCCTCNGGCTGNATNGCGC
TNNTTAATGACGGCTNGTTTCTTTTCTGTGNTGCNNGAAGCCTTGNGGGG
NTCCNGGGAGGNCCNNTTGN

According to some embodiments, p141_EXPR_AAV_CBA-BFP_Antisense_miRNA1_11-ATTB2 (908 bp) comprises SEQ ID NO: 76, shown below.

NNNNNNNNNNNNNGNGNGNGGCAGATCTGGGCCATTTGTTCCNTGTGAGT
GCTAGTAACAGGCCTTGTGTCTTCAGTGTCAGTTTCATACGTCAGTCAGT
GGCCAAAACGTATGAAAGGCTGACACTGAAAGCATACAGCCTTCAGCAAG
CCTCCAGGATCCACTGGTCGACTCACTACCTCCCTTTAATACAACTTTTG
TATACAAAGTTGTCTAGGAACTACCTACCTTACGCTTCTTCTTTGGAGCT
CCCTCATTAAGCTTGTGCCCCAGTTTGCTAGGGAGGTCGCAGTATCTGGC
CACTGCCACCTCGTGCTGCTCGACGTAGGTCTCGTTGTTGGCCTCCTTGA
TTCTTTCCAGTCTGTAGTCCACATAGTAGACGCCAGGCATCTTGAGGTTC
TTAGCGGGTTTCTTGGATCTATATGTGGTCTTGATGTTTGCGATCAGATG
GCTCCCGCCCACGAGCTTCAGGGCCATGTCGTTTCTGCCTTCCAGGCCGC
CGTCAGCGGGGTACAGCGTCTCGGTGAAGGCCTCCCAGCCGAGTGTTTTC
TTCTGCATCACAGGGCCGTTGGATGTGAAGTTCACCCCTCTGATCTTGAC
GTTGTAGATGAGGCAGCCGTCCTGGAGGCTGGTGTCCTGGGTAGCGGTCA
GCACGCCCCCGTCTTCGTATGTGGTGACTCTCTCCCATGTGAAGCCCTCA
GGGAAGGACTGCTTGAAGAAGTCGGGGATGCCCTGGGTGTGGTTGATGAA
GGTCTTGCTGCCGTAGAGGAAGCTAGTAGCCAGGATGTCGAAGGCGAAGG
GGAGAGGGCCGCCCTCGACCACCTTGATTCTCATGGTCTGGGTGCCCTCG
TAGGGCTTGCCTTCGCCCTCGGATGTGCACTTGAAGTGATGNTTGTCCAC
GGTGCCNN

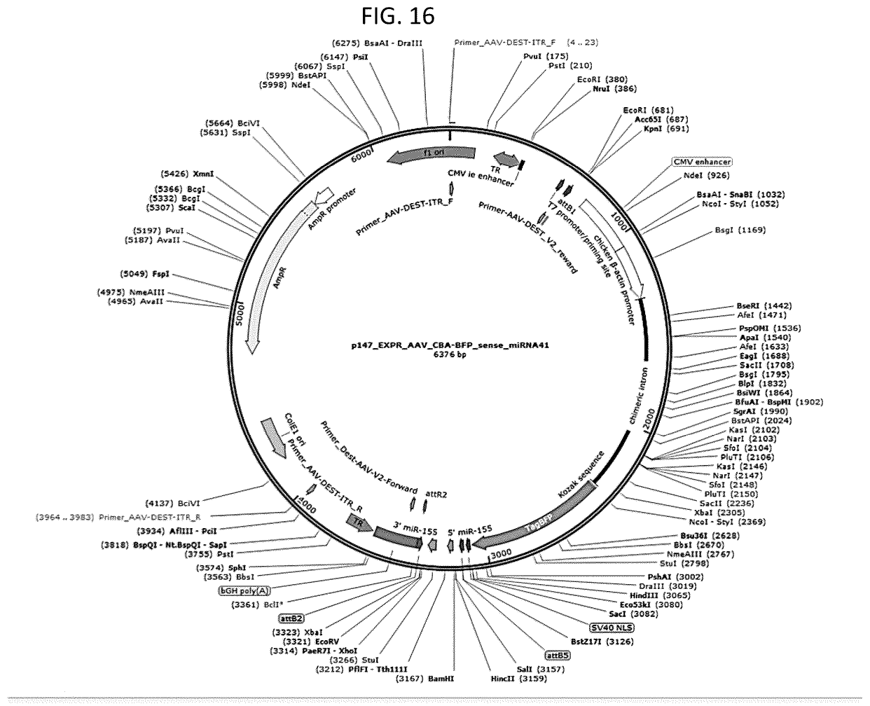

(2) p147_EXPR_AAV_CBA-BFP_sense_miRNA41. This construct comprises CBA promoter, BFP sequence, miRNA41 targeting sense C9orf72, bGH polyA signal. Ampicillin resistance gene. The vector map is shown in FIG. 16. According to some embodiments, the nucleic acid sequence of p147_EXPR_AAV_CBA-BFP_sense_miRNA41 comprises SEQ ID NO: 77. According to some embodiments, the nucleic acid sequence of p147_EXPR_AAV_CBA-BFP_sense_miRNA41 is at least 85%, at least 90%, at least 95%, at least 96%, at least 97%, at least 98%, at least 99% identical to SEQ ID NO: 77, shown below.

ccggcgaacgtggcgagaaaggaagggaagaaagcgaaaggagcgggcgc
tagggcgctggcaagtgtagcggtcacgctgcgcgtaaccaccacacccg
ccgcgcttaatgcgccgctacagggcgcgtcgcgccattcgccattcagg
ctacgcaactgttgggaagggcgatcggtgcgggcctcttcgctattacg
ccaggctgcaggggggggggggggggggttggccactccctctctgcgcg
ctcgctcgctcactgaggccgggcgaccaaaggtcgcccgacgcccgggc
tttgcccgggcggcctcagtgagcgagcgagcgcgcagagagggagtggc
caactccatcactaggggttcctagatctgaattcgcgacggatcgggag
atctcccgatcccctatggtgcactctcagtacaatctgctctgatgccg
catagttaagccagtatctgctccctgcttgtgtgttggaggtcgctgag
tagtgcgcgagcaaaatttaagctacaacaaggcaaggcttgaccgacaa
ttctctggctaactagagaacccactgcttactggcttatcgaaattaat
acgactcactatagggagacccaagctggctagttaagctatcaacaagt
ttGTACAAAAAAGCAGGCTTACTCAGATCTGAATTCGGTACCTAGTTATT
AATAGTAATCAATTACGGGGTCATTAGTTCATAGCCCATATATGGAGTTC
CGCGTTACATAACTTACGGTAAATGGCCCGCCTGGCTGACCGCCCAACGA
CCCCCGCCCATTGACGTCAATAATGACGTATGTTCCCATAGTAACGCCAA
TAGGGACTTTCCATTGACGTCAATGGGTGGAGTATTTACGGTAAACTGCC
CACTTGGCAGTACATCAAGTGTATCATATGCCAAGTACGCCCCCTATTGA
CGTCAATGACGGTAAATGGCCCGCCTGGCATTATGCCCAGTACATGACCT
TATGGGACTTTCCTACTTGGCAGTACATCTACGTATTAGTCATCGCTATT
ACCATGGTCGAGGTGAGCCCCACGTTCTGCTTCACTCTCCCCATCTCCCC
CCCCTCCCCACCCCCAATTTTGTATTTATTTATTTTTTAATTATTTTGTG
CAGCGATGGGGGCGGGGGGGGGGGGGGGGCGCGCGCCAGGCGGGGCGGGG
CGGGGCGAGGGGCGGGGCGGGGCGAGGCGGAGAGGTGCGGCGGCAGCCAA
TCAGAGCGGCGCGCTCCGAAAGTTTCCTTTTATGGCGAGGCGGCGGCGGC
GGCGGCCCTATAAAAAGCGAAGCGCGCGGCGGGCGGGAGTCGCTGCGCGC
TGCCTTCGCCCCGTGCCCCGCTCCGCCGCCGCCTCGCGCCGCCCGCCCCG
GCTCTGACTGACCGCGTTACTCCCACAGGTGAGCGGGCGGGACGGCCCTT
CTCCTCCGGGCTGTAATTAGCGCTTGGTTTAATGACGGCTTGTTTCTTTT
CTGTGGCTGCGTGAAAGCCTTGAGGGGCTCCGGGAGGGCCCTTTGTGCGG
GGGGAGCGGCTCGGGGGGTGCGTGCGTGTGTGTGTGCGTGGGGAGCGCCG
CGTGCGGCTCCGCGCTGCCCGGCGGCTGTGAGCGCTGCGGGCGCGGCGCG
GGGCTTTGTGCGCTCCGCAGTGTGCGCGAGGGGAGCGCGGCCGGGGGCGG
TGCCCCGCGGTGCGGGGGGGGCTGCGAGGGGAACAAAGGCTGCGTGCGGG
GTGTGTGCGTGGGGGGGTGAGCAGGGGGTGTGGGCGCGTCGGTCGGGCTG
CAACCCCCCCTGCACCCCCCTCCCCGAGTTGCTGAGCACGGCCCGGCTTC
GGGTGCGGGGCTCCGTACGGGGCGTGGCGCGGGGCTCGCCGTGCCGGGCG
GGGGGTGGCGGCAGGTGGGGGTGCCGGGCGGGGCGGGGCCGCCTCGGGCC
GGGGAGGGCTCGGGGGAGGGGCGCGGCGGCCCCCGGAGCGCCGGCGGCTG
TCGAGGCGCGGCGAGCCGCAGCCATTGCCTTTTATGGTAATCGTGCGAGA
GGGCGCAGGGACTTCCTTTGTCCCAAATCTGTGCGGAGCCGAAATCTGGG
AGGCGCCGCCGCACCCCCTCTAGCGGGCGCGGGGCGAAGCGGTGCGGCGC
CGGCAGGAAGGAAATGGGCGGGGAGGGCCTTCGTGCGTCGCCGCGCCGCC
GTCCCCTTCTCCCTCTCCAGCCTCGGGGCTGTCCGCGGGGGGACGGCTGC
CTTCGGGGGGGACGGGGCAGGGCGGGGTTCGGCTTCTGGCGTGTGACCGG
CGGCTCTAGAGCCTCTGCTAACCATGTTCATGCCTTCTTCTTTTTCCTAC
AGCTCCTGGGCAACGCCACCATGGATGAGCGAGCTGATTAAGGAGAACAT
GCACATGAAGCTGTACATGGAGGGCACCGTGGACAACCATCACTTCAAGT
GCACATCCGAGGGCGAAGGCAAGCCCTACGAGGGCACCCAGACCATGAGA
ATCAAGGTGGTCGAGGGCGGCCCTCTCCCCTTCGCCTTCGACATCCTGGC
TACTAGCTTCCTCTACGGCAGCAAGACCTTCATCAACCACACCCAGGGCA
TCCCCGACTTCTTCAAGCAGTCCTTCCCTGAGGGCTTCACATGGGAGAGA
GTCACCACATACGAAGACGGGGGCGTGCTGACCGCTACCCAGGACACCAG
CCTCCAGGACGGCTGCCTCATCTACAACGTCAAGATCAGAGGGGTGAACT
TCACATCCAACGGCCCTGTGATGCAGAAGAAAACACTCGGCTGGGAGGCC
TTCACCGAGACGCTGTACCCCGCTGACGGCGGCCTGGAAGGCAGAAACGA
CATGGCCCTGAAGCTCGTGGGCGGGAGCCATCTGATCGCAAACATCAAGA
CCACATATAGATCCAAGAAACCCGCTAAGAACCTCAAGATGCCTGGCGTC
TACTATGTGGACTACAGACTGGAAAGAATCAAGGAGGCCAACAACGAGAC
CTACGTCGAGCAGCACGAGGTGGCAGTGGCCAGATACTGCGACCTCCCTA
GCAAACTGGGGCACAAGCTTAATGAGGGAGCTCCAAAGAAGAAGCGTAAG
GTAGGTAGTTCCTAGACAACTTTGTATACAAAAGTTGTATTAAAGGGAGG
TAGTGAGTCGACCAGTGGATCCTGGAGGCTTGCTGAAGGCTGTATGCTTA
GTATGTATGACAAAGTCCTGTTTTGGCCACTGACTGACAGGACTTTCATA
CATACTAGACACAAGGCCTGTTACTAGCACTCACATGGAACAAATGGCCC
AGATCTGGCCGCACTCGAGATATCTAGAACCCAGCTTTcttgtacaaagt
ggttgatcgctgatcagcctcgactgtgccttctagttgccagccatctg
ttgtttgcccctcccccgtgccttccttgaccctggaaggtgccactccc
actgtcctttcctaataaaatgaggaaattgcatcgcattgtctgagtag
gtgtcattctattctggggggtggggtggggcaggacagcaagggggagg
attgggaagacaatagcaggcatgctggggagagatctaggaacccctag
tgatggagttggccactccctctctgcgcgctcgctcgctcactgaggcc
gcccgggcaaagcccgggcgtcgggcgacctttggtcgcccggcctcagt
gagcgagcgagcgcgcagagagggagtggccaaccccccccccccccccc
ctgcagccctgcattaatgaatcggccaacgcgcggggagaggcggtttg
cgtattgggcgctcttccgcttcctcgctcactgactcgctgcgctcggt
cgttcggctgcggcgagcggtatcagctcactcaaaggcggtaatacggt
tatccacagaatcaggggataacgcaggaaagaacatgtgagcaaaaggc
cagcaaaaggccaggaaccgtaaaaaggccgcgttgctggcgtttttcca
taggctccgcccccctgacgagcatcacaaaaatcgacgctcaagtcaga
ggtggcgaaacccgacaggactataaagataccaggcgtttccccctgga
agctccctcgtgcgctctcctgttccgaccctgccgcttaccggatacct
gtccgcctttctcccttcgggaagcgtggcgctttctcaatgctcacgct
gtaggtatctcagttcggtgtaggtcgttcgctccaagctgggctgtgtg
cacgaaccccccgttcagcccgaccgctgcgccttatccggtaactatcg
tcttgagtccaacccggtaagacacgacttatcgccactggcagcagcca
ctggtaacaggattagcagagcgaggtatgtaggcggtgctacagagttc
ttgaagtggtggcctaactacggctacactagaaggacagtatttggtat
ctgcgctctgctgaagccagttaccttcggaaaaagagttggtagctctt
gatccggcaaacaaaccaccgctggtagcggtggtttttttgtttgcaag
cagcagattacgcgcagaaaaaaaggatctcaagaagatcctttgatctt
ttctacggggtctgacgctcagtggaacgaaaactcacgttaagggattt
tggtcatgagattatcaaaaaggatcttcacctagatccttttaaattaa
aaatgaagttttaaatcaatctaaagtatatatgagtaaacttggtctga
cagttaccaatgcttaatcagtgaggcacctatctcagcgatctgtctat
ttcgttcatccatagttgcctgactccccgtcgtgtagataactacgata
cgggagggcttaccatctggccccagtgctgcaatgataccgcgagaccc
acgctcaccggctccagatttatcagcaataaaccagccagccggaaggg
ccgagcgcagaagtggtcctgcaactttatccgcctccatccagtctatt
aattgttgccgggaagctagagtaagtagttcgccagttaatagtttgcg
caacgttgttgccattgctacaggcatcgtggtgtcacgctcgtcgtttg
gtatggcttcattcagctccggttcccaacgatcaaggcgagttacatga
tcccccatgttgtgcaaaaaagcggttagctccttcggtcctccgatcgt
tgtcagaagtaagttggccgcagtgttatcactcatggttatggcagcac
tgcataattctcttactgtcatgccatccgtaagatgcttttctgtgact
ggtgagtactcaaccaagtcattctgagaatagtgtatgcggcgaccgag
ttgctcttgcccggcgtcaatacgggataataccgcgccacatagcagaa
ctttaaaagtgctcatcattggaaaacgttcttcggggcgaaaactctca
aggatcttaccgctgttgagatccagttcgatgtaacccactcgtgcacc
caactgatcttcagcatcttttactttcaccagcgtttctgggtgagcaa
aaacaggaaggcaaaatgccgcaaaaaagggaataagggcgacacggaaa
tgttgaatactcatactcttcctttttcaatattattgaagcatttatca
gggttattgtctcatgagcggatacatatttgaatgtatttagaaaaata
aacaaataggggttccgcgcacatttccccgaaaagtgccacctgacgtc
taagaaaccattattatcatgacattaacctataaaaataggcgtatcac
gaggccctttcgtctcgcgcgtttcggtgatgacggtgaaaacctctgac
acatgcagctcccggagacggtcacagcttgtctgtaagcggatgccggg
agcagacaagcccgtcagggcgcgtcagcgggtgttggcgggtgtcgggg
ctggcttaactatgcggcatcagagcagattgtactgagagtgcaccata
tgcggtgtgaaataccgcacagatgcgtaaggagaaaataccgcatcagg
aaattgtaaacgttaatattttgttaaaattcgcgttaaatttttgttaa
atcagctcattttttaaccaataggccgaaatcggcaaaatcccttataa
atcaaaagaatagaccgagatagggttgagtgttgttccagtttggaaca
agagtccactattaaagaacgtggactccaacgtcaaagggcgaaaaacc
gtctatcagggcgatggcccactacgtgaaccatcaccctaatcaagttt
tttggggtcgaggtgccgtaaagcactaaatcggaaccctaaagggagcc
cccgatttagagcttgacggggaaag

According to some embodiments, p147_EXPR_AAV_CBA-BFP_sense_miRNA41_attb1_Sequencing result (953 bp) comprises SEQ ID NO: 78, shown below.

NNNNNNNNNNNNNNGNNNNNNGTTATTAATAGTAATCAATTACGGGGTCA
TTAGTTCATAGCCCATATATGGAGTTCCNCGTTACATAACTTACGGTAAA
TGGCCCGCCTGGCTGACCGCCCAACGACCCCCGCCCATTGACGTCAATAA
TGACGTATGTTCCCATAGTAACGCCAATAGGGACTTTCCATTGACGTCAA
TGGGTGGAGTATTTACGGTAAACTGCCCACTTGGCAGTACATCAAGTGTA
TCATATGCCAAGTACGCCCCCTATTGACGTCAATGACGGTAAATGGCCCG
CCTGGCATTATGCCCAGTACATGACCTTATGGGACTTTCCTACTTGGCAG
TACATCTACGTATTAGTCATCGCTATTACCATGGTCGAGGTGAGCCCCAC
GTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGG
GGGGGGCGCGCGCCAGGCGGGGCGGGGCGGGGCNAGGGGCGGGGCGGGGC
GAGGCGAAAAGGTGCGGCGGCAGCCAATCAGAGCGGCGCGCTCCGAAAGT
TTCCTTTTATGGCGAGGCGGCGGCGGCGGCGGCCCTATAAAAAGNNAAGC
GCGCGGCGGGCGGGAGTCGCTGCGCGCTGCCTTCGCCCCGTGCCCCGCTC
CGCCGCCGCCTCGCGCCGCCCGCCCCGGCTCTGACTGACCGCGTTACTCC
CACAGGTGAGCGGGCGGNACGNCCCTTCTCCTCCGGGCTGTAATTAGCGC
TTNNTTAATGACGGCTTGTTCNTTTCTGNNGCTGNNNAAAGCCTTGNGGG
GCTNNNAGGNCNTTTGNNNGGGGNAGNGNTCGGGGNNNNNNNTGNNTNTN
TNNNGNANCNCCNNGTGNGNTCCNNNCTGCCCGNGCTNNNACNCTGNNNN
CNN

According to some embodiments, p141_EXPR_AAV_CBA-BFP_Antisense_miRNA1_M_5-ATTB2 (958 bp) comprises SEQ ID NO: 79, shown below.

CNNNNNNNNNNNNNNNGNNGCAGATCTGGGCCATTTGTTCCATGTGAGTG
CTAGTAACAGGCCTTGTGTCTAGTATGTANGAAAGTCCTGTCAGTCAGTG
GCCAAAACAGGACTTTGTCATACATACTAAGCATACAGCCTTCAGCAAGC
CTCCAGGATCCACTGGTCGACTCACTACCTCCCTTTAATACAACTTTTGT
ATACAAAGTTGTCTAGGAACTACCTACCTTACGCTTCTTCTTTGGAGCTC
CCTCATTAAGCTTGTGCCCCAGTTTGCTAGGGAGGTCGCAGTATCTGGCC
ACTGCCACCTCGTGCTGCTCGACGTAGGTCTCGTTGTTGGCCTCCTTGAT
TCTTTCCAGTCTGTAGTCCACATAGTAGACGCCAGGCATCTTGAGGTTCT
TAGCGGGTTTCTTGGATCTATATGTGGTCTTGATGTTTGCGATCAGATGG
CTCCCGCCCACGAGCTTCAGGGCCATGTCGTTTCTGCCTTCCAGGCCGCC
GTCAGCGGGGTACAGCGTCTCGGTGAAGGCCTCCCAGCCGAGTGTTTTCT
TCTGCATCACAGGGCCGTTGGATGTGAAGTTCACCCCTCTGATCTTGACG
TTGTAGATGAGGCAGCCGTCCTGGAGGCTGGTGTCCTGGGTAGCGGTCAG
CACGCCCCCGTCTTCGTATGTGGTGACTCTCTCCCATGTGAAGCCCTCAG
GGAAGGACTGCTTGAAGAAGTCGGGGATGCCCTGGGTGTGGTTGATGAAG
GTCTTGCTGCCGTAGAGGAAGCTAGTAGCCAGGATGTCGAAGGCGAAGGG
GAGAGGGCCGCCCTCGACCACCTTGATTCTCATGGTCTGGGTGCCCTCGT
AGGGCTTGCCTTCGCCCTCGGATGTGCACTTGAAGTGATGGTTGTCCACG
GTGCCCTCCATGTACAGCTTCATGTGCATGTTCTNCCTTAATCAGCTCGC
TCATCCAN

Reporter with Target Tandem Arrays (Puro+) Transfection in HEK293 Cells.

Next, tandem array constructs were prepared. Use of Puro+ ensured only cells that were transduced with reporter constructs survived. Use of BSD+ ensured only cells that were transduced with miRNA constructs survived. Double selection ensured accurate knock-down efficiency.

The Following Tandem Array Constructs were Prepared:

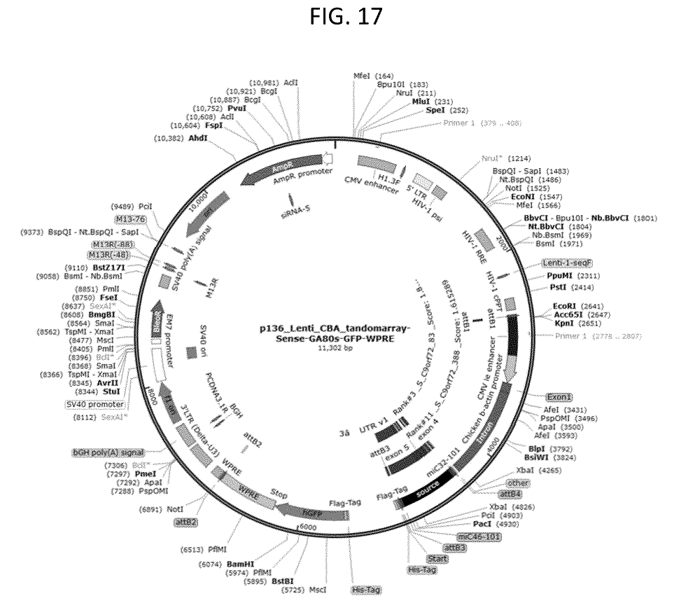

(1) p136_Lenti_CBA_tandomarray-Sense-GA80s-GFP-WPRE. This construct comprises CBA promoter, tandomArray-sense (miRNA targeting site C9orf72 on sense sequence), Glycine Alanine repeat sequence tagged with GFP gene, WPRE, Ampicillin resistance gene, lentivirus production gene. The vector map is shown in FIG. 17. According to some embodiments, the nucleic acid sequence of p136_Lenti_CBA_tandomarray-Sense-GA80s-GFP-WPRE comprises SEQ ID NO: 80. According to some embodiments, the nucleic acid sequence of p136_Lenti_CBA_tandomarray-Sense-GA80s-GFP-WPRE is at least 85%, at least 90%, at least 95%, at least 96%, at least 97%, at least 98%, at least 99% identical to SEQ ID NO: 80, shown below.

gtcgacggatcgggagatctcccgatcccctatggtgcactctcagtacaatctgctctgatgc
cgcatagttaagccagtatctgctccctgcttgtgtgttggaggtcgctgagtagtgcgcgagc
aaaatttaagctacaacaaggcaaggcttgaccgacaattgcatgaagaatctgcttagggtta
ggcgttttgcgctgcttcgcgatgtacgggccagatatacgcgttgacattgattattgactag
ttattaatagtaatcaattacggggtcattagttcatagcccatatatggagttccgcgttaca
taacttacggtaaatggcccgcctggctgaccgcccaacgacccccgcccattgacgtcaataa
tgacgtatgttcccatagtaacgccaatagggactttccattgacgtcaatgggtggagtattt
acggtaaactgcccacttggcagtacatcaagtgtatcatatgccaagtacgccccctattgac
gtcaatgacggtaaatggcccgcctggcattatgcccagtacatgaccttatgggactttccta
cttggcagtacatctacgtattagtcatcgctattaccatggtgatgcggttttggcagtacat
caatgggcgtggatagcggtttgactcacggggatttccaagtctccaccccattgacgtcaat
gggagtttgttttggcaccaaaatcaacgggactttccaaaatgtcgtaacaactccgccccat
tgacgcaaatgggcggtaggcgtgtacggtgggaggtctatataagcagcgcgttttgcctgta
ctgggtctctctggttagaccagatctgagcctgggagctctctggctaactagggaacccact
gcttaagcctcaataaagcttgccttgagtgcttcaagtagtgtgtgcccgtctgttgtgtgac
tctggtaactagagatccctcagacccttttagtcagtgtggaaaatctctagcagtggcgccc
gaacagggacttgaaagcgaaagggaaaccagaggagctctctcgacgcaggactcggcttgct
gaagcgcgcacggcaagaggcgaggggcggcgactggtgagtacgccaaaaattttgactagcg
gaggctagaaggagagagatgggtgcgagagcgtcagtattaagcgggggagaattagatcgcg
atgggaaaaaattcggttaaggccagggggaaagaaaaaatataaattaaaacatatagtatgg
gcaagcagggagctagaacgattcgcagttaatcctggcctgttagaaacatcagaaggctgta
gacaaatactgggacagctacaaccatcccttcagacaggatcagaagaacttagatcattata
taatacagtagcaaccctctattgtgtgcatcaaaggatagagataaaagacaccaaggaagct
ttagacaagatagaggaagagcaaaacaaaagtaagaccaccgcacagcaagcggccgctgatc
ttcagacctggaggaggagatatgagggacaattggagaagtgaattatataaatataaagtag
taaaaattgaaccattaggagtagcacccaccaaggcaaagagaagagtggtgcagagagaaaa
aagagcagtgggaataggagctttgttccttgggttcttgggagcagcaggaagcactatgggc
gcagcgtcaatgacgctgacggtacaggccagacaattattgtctggtatagtgcagcagcaga
acaatttgctgagggctattgaggcgcaacagcatctgttgcaactcacagtctggggcatcaa
gcagctccaggcaagaatcctggctgtggaaagatacctaaaggatcaacagctcctggggatt
tggggttgctctggaaaactcatttgcaccactgctgtgccttggaatgctagttggagtaata
aatctctggaacagatttggaatcacacgacctggatggagtgggacagagaaattaacaatta
cacaagcttaatacactccttaattgaagaatcgcaaaaccagcaagaaaagaatgaacaagaa
ttattggaattagataaatgggcaagtttgtggaattggtttaacataacaaattggctgtggt
atataaaattattcataatgatagtaggaggcttggtaggtttaagaatagtttttgctgtact
ttctatagtgaatagagttaggcagggatattcaccattatcgtttcagacccacctcccaacc
ccgaggggacccgacaggcccgaaggaatagaagaagaaggtggagagagagacagagacagat
ccattcgattagtgaacggatcggcactgcgtgcgccaattctgcagacaaatggcagtattca
tccacaattttaaaagaaaaggggggattggggggtacagtgcaggggaaagaatagtagacat
aatagcaacagacatacaaactaaagaattacaaaaacaaattacaaaaattcaaaattttcgg
gtttattacagggacagcagagatccagtttggttaatggCCGCacaagtttGTACAAAAAAGC
AGGCTTActcagatctgaattcggtacctagttattaatagtaatcaattacggggtcattagt
tcatagcccatatatggagttccgcgttacataacttacggtaaatggcccgcctggctgaccg
cccaacgacccccgcccattgacgtcaataatgacgtatgttcccatagtaacgccaataggga
ctttccattgacgtcaatgggtggagtatttacggtaaactgcccacttggcagtacatcaagt
gtatcatatgccaagtacgccccctattgacgtcaatgacggtaaatggcccgcctggcattat
gcccagtacatgaccttatgggactttcctacttggcagtacatctacgtattagtcatcgcta
ttaccatggtcgaggtgagccccacgttctgcttcactctccccatctcccccccctccccacc
cccaattttgtatttatttattttttaattattttgtgcagcgatgggggcggggggggggggg
gggcgcgcgccaggcggggcggggcggggcgaggggcggggcggggcgaggcggagaggtgcgg
cggcagccaatcagagcggcgcgctccgaaagtttccttttatggcgaggcggcggcggcggcg
gccctataaaaagcgaagcgcgcggcgggcgggagtcgctgcgcgctgccttcgccccgtgccc
cgctccgccgccgcctcgcgccgcccgccccggctctgactgaccgcgttactcccacaggtga
gcgggcgggacggcccttctcctccgggctgtaattagcgcttggtttaatgacggcttgtttc
ttttctgtggctgcgtgaaagccttgaggggctccgggagggccctttgtgcggggggagcggc
tcggggggtgcgtgcgtgtgtgtgtgcgtggggagcgccgcgtgcggctccgcgctgcccggcg
gctgtgagcgctgcgggcgcggcgcggggctttgtgcgctccgcagtgtgcgcgaggggagcgc
ggccgggggcggtgccccgcggtgcggggggggctgcgaggggaacaaaggctgcgtgcggggt
gtgtgcgtgggggggtgagcagggggtgtgggcgcgtcggtcgggctgcaaccccccctgcacc
cccctccccgagttgctgagcacggcccggcttcgggtgcggggctccgtacggggcgtggcgc
ggggctcgccgtgccgggcggggggtggcggcaggtgggggtgccgggcggggcggggccgcct
cgggccggggagggctcgggggaggggcgcggcggcccccggagcgccggcggctgtcgaggcg
cggcgagccgcagccattgccttttatggtaatcgtgcgagagggcgcagggacttcctttgtc
ccaaatctgtgcggagccgaaatctgggaggcgccgccgcaccccctctagcgggcgcggggcg
aagcggtgcggcgccggcaggaaggaaatgggcggggagggccttcgtgcgtcgccgcgccgcc
gtccccttctccctctccagcctcggggctgtccgcggggggacggctgccttcgggggggacg
gggcagggcggggttcggcttctggcgtgtgaccggcggctctagagcctctgctaaccatgtt
catgccttcttctttttcctacagctcctgggcaacgccaccatggCACCCAACTTTTCTATAC
AAAGTTGTATCCTTACTCTAGGACCAAGAATGAACTGCTTTCATCTATGAAAGAAGAAATAGAT
GTAAGTTTAAATGAGAGCAATTATACACTTTAATGTATATTATTAATATTCTAAACATACTATT
CACATACAGTAATAGGAGCAATTAATATTTAATGTAGTGTCTTTTGAAACAAAAGAGTGTTAAG
AGATACCTTTAGAAGAGGAAGTTGTTCTTGTAAAAAAAAGTGTTATTTCAACACTATGATACAG
TACTCAATGATGATGATAAAGTAAGAATTTTTCTTTTCATAAAATAGGGACATTACGTATTTGA
ACACTCATTATATTTCTATATATAACAGAATCCTTTCATATTAAGTTGTACTGTAGATGAACTT
AAGTTATTTAAGCAGTGGAGTTTAGTACTTAATATAAGCATTGAGTAAGATAAATAATATAAAA
GCTAACATTTCCTATTTACATTTCTTCTAGACACAGTTACAGATTTTCATGAAATTTTAGCATG
AGTGTGTTTAACCTAAAGCCTTTCATACATCATTTTAAACATGTCAATTTCTTCAGCTACATTA
ATTAAATGATATTATATTATCTTCAGGTTCCGAAGAGAACAACTTTGTATAATAAAGTTGTAAT
GCATCACCACCATCATCACGATTATAAGGATGACGATGACAAGGGAGCTGGGGCGGGTGCGGGG
GCAGGAGCCGGAGCCGGCGCGGGCGCAGGTGCAGGTGCTGGTGCTGGCGCCGGTGCGGGAGCCG
GGGCAGGCGCTGGGGCGGGCGCTGGTGCTGGTGCTGGTGCCGGGGCCGGCGCCGGAGCAGGGGC
TGGAGCGGGCGCGGGGGCGGGCGCCGGAGCCGGTGCGGGGGCCGGGGCCGGCGCAGGCGCAGGC
GCTGGCGCCGGTGCTGGAGCTGGCGCCGGGGCGGGAGCAGGGGCCGGAGCAGGCGCTGGTGCCG
GCGCAGGGGCTGGCGCGGGGGCAGGTGCAGGCGCAGGTGCCGGTGCCGGGGCAGGCGCTGGCGC
TGGTGCCGGCGCAGGGGCAGGGGCAGGAGCGGGCGCAGGTGCGGGGGCTGGTGCCGGTGCTGGA
GCTGGGGCAGGGGCGGGCGCAGGTGCCGGCGCGGGTGCCGGTGCCGGCGCCGGGGCCGGGGCCG
GGGCAGGCGCTCATCACCACCATCATCACGATTATAAGGATGACGATGACAAGagcaagggcga
ggaactgttcactggcgtggtcccaattctcgtggaactggatggcgatgtgaatgggcacaaa
ttttctgtcagcggagagggtgaaggtgatgccacatacggaaagctcaccctgaaattcatct
gcaccactggaaagctccctgtgccatggccaacactggtcactaccctgacctatggcgtgca
gtgcttttccagatacccagaccatatgaagcagcatgactttttcaagagcgccatgcccgag
ggctatgtgcaggagagaaccatctttttcaaagatgacgggaactacaagacccgcgctgaag
tcaagttcgaaggtgacaccctggtgaatagaatcgagctgaagggcattgactttaaggagga
tggaaacattctcggccacaagctggaatacaactataactcccacaatgtgtacatcatggcc
gacaagcaaaagaatggcatcaaggtcaacttcaagatcagacacaacattgaggatggatccg
tgcagctggccgaccattatcaacagaacactccaatcggcgacggccctgtgctcctcccaga
caaccattacctgtccacccagtctgccctgtctaaagatcccaacgaaaagagagaccacatg
gtcctgctggagtttgtgaccgctgctgggatcacacatggcatggacgagctgtacaagTGAa
atcaacctctggattacaaaatttgtgaaagattgactggtattcttaactatgttgctccttt
tacgctatgtggatacgctgctttaatgcctttgtatcatgctattgcttcccgtatggctttc
attttctcctccttgtataaatcctggttgctgtctctttatgaggagttgtggcccgttgtca
ggcaacgtggcgtggtgtgcactgtgtttgctgacgcaacccccactggttggggcattgccac
cacctgtcagctcctttccgggactttcgctttccccctccctattgccacggcggaactcatc
gccgcctgccttgcccgctgctggacaggggctcggctgttgggcactgacaattccgtggtgt
tgtcggggaaatcatcgtcctttccttggctgctcgcctgtgttgccacctggattctgcgcgg
gacgtccttctgctacgtcccttcggccctcaatccagcggaccttccttcccgcggcctgctg
ccggctctgcggcctcttccgcgtcttcgccttcgccctcagacgagtcggatctccctttggg
ccgcctccccgcctgAACCCAGCTTTcttgtacaaagtggtGCGGccgcggcctgctgccggct
ctgcggcctcttccgcgtcttcgccttcgccctcagacgagtcggatctccctttgggccgcct
ccccgcgtcgactttaagaccaatgacttacaaggcagctgtagatcttagccactttttaaaa
gaaaaggggggactggaagggctaattcactcccaacgaagacaagatctgctttttgcttgta
ctgggtctctctggttagaccagatctgagcctgggagctctctggctaactagggaacccact
gcttaagcctcaataaagcttgccttgagtgcttcaagtagtgtgtgcccgtctgttgtgtgac
tctggtaactagagatccctcagacccttttagtcagtgtggaaaatctctagcagggcccgtt
taaacccgctgatcagcctcgactgtgccttctagttgccagccatctgttgtttgcccctccc
ccgtgccttccttgaccctggaaggtgccactcccactgtcctttcctaataaaatgaggaaat
tgcatcgcattgtctgagtaggtgtcattctattctggggggtggggtggggcaggacagcaag
ggggaggattgggaagacaatagcaggcatgctggggatgcggtgggctctatggcttctgagg
cggaaagaaccagctggggctctagggggtatccccacgcgccctgtagcggcgcattaagcgc
ggcgggtgtggtggttacgcgcagcgtgaccgctacacttgccagcgccctagcgcccgctcct
ttcgctttcttcccttcctttctcgccacgttcgccggctttccccgtcaagctctaaatcggg
ggctccctttagggttccgatttagtgctttacggcacctcgaccccaaaaaacttgattaggg
tgatggttcacgtagtgggccatcgccctgatagacggtttttcgccctttgacgttggagtcc
acgttctttaatagtggactcttgttccaaactggaacaacactcaaccctatctcggtctatt
cttttgatttataagggattttgccgatttcggcctattggttaaaaaatgagctgatttaaca
aaaatttaacgcgaattaattctgtggaatgtgtgtcagttagggtgtggaaagtccccaggct
ccccagcaggcagaagtatgcaaagcatgcatctcaattagtcagcaaccaggtgtggaaagtc
cccaggctccccagcaggcagaagtatgcaaagcatgcatctcaattagtcagcaaccatagtc
ccgcccctaactccgcccatcccgcccctaactccgcccagttccgcccattctccgccccatg
gctgactaattttttttatttatgcagaggccgaggccgcctctgcctctgagctattccagaa
gtagtgaggaggcttttttggaggcctaggcttttgcaaaaagctcccgggagcttgtatatcc
attttcggatctgatcagcacgtgttgacaattaatcatcggcatagtatatcggcatagtata
atacgacaaggtgaggaactaaaccatggccaagttgaccagtgccgttccggtgctcaccgcg
cgcgacgtcgccggagcggtcgagttctggaccgaccggctcgggttctcccgggacttcgtgg
aggacgacttcgccggtgtggtccgggacgacgtgaccctgttcatcagcgcggtccaggacca
ggtggtgccggacaacaccctggcctgggtgtgggtgcgcggcctggacgagctgtacgccgag
tggtcggaggtcgtgtccacgaacttccgggacgcctccgggccggccatgaccgagatcggcg
agcagccgtgggggcgggagttcgccctgcgcgacccggccggcaactgcgtgcacttcgtggc
cgaggagcaggactgacacgtgctacgagatttcgattccaccgccgccttctatgaaaggttg
ggcttcggaatcgttttccgggacgccggctggatgatcctccagcgcggggatctcatgctgg
agttcttcgcccaccccaacttgtttattgcagcttataatggttacaaataaagcaatagcat
cacaaatttcacaaataaagcatttttttcactgcattctagttgtggtttgtccaaactcatc
aatgtatcttatcatgtctgtataccgtcgacctctagctagagcttggcgtaatcatggtcat
agctgtttcctgtgtgaaattgttatccgctcacaattccacacaacatacgagccggaagcat
aaagtgtaaagcctggggtgcctaatgagtgagctaactcacattaattgcgttgcgctcactg
cccgctttccagtcgggaaacctgtcgtgccagctgcattaatgaatcggccaacgcgcgggga
gaggcggtttgcgtattgggcgctcttccgcttcctcgctcactgactcgctgcgctcggtcgt
tcggctgcggcgagcggtatcagctcactcaaaggcggtaatacggttatccacagaatcaggg
gataacgcaggaaagaacatgtgagcaaaaggccagcaaaaggccaggaaccgtaaaaaggccg
cgttgctggcgtttttccataggctccgcccccctgacgagcatcacaaaaatcgacgctcaag
tcagaggtggcgaaacccgacaggactataaagataccaggcgtttccccctggaagctccctc
gtgcgctctcctgttccgaccctgccgcttaccggatacctgtccgcctttctcccttcgggaa
gcgtggcgctttctcatagctcacgctgtaggtatctcagttcggtgtaggtcgttcgctccaa
gctgggctgtgtgcacgaaccccccgttcagcccgaccgctgcgccttatccggtaactatcgt
cttgagtccaacccggtaagacacgacttatcgccactggcagcagccactggtaacaggatta
gcagagcgaggtatgtaggcggtgctacagagttcttgaagtggtggcctaactacggctacac
tagaagaacagtatttggtatctgcgctctgctgaagccagttaccttcggaaaaagagttggt
agctcttgatccggcaaacaaaccaccgctggtagcggtggtttttttgtttgcaagcagcaga
ttacgcgcagaaaaaaaggatctcaagaagatcctttgatcttttctacggggtctgacgctca
gtggaacgaaaactcacgttaagggattttggtcatgagattatcaaaaaggatcttcacctag
atccttttaaattaaaaatgaagttttaaatcaatctaaagtatatatgagtaaacttggtctg
acagttaccaatgcttaatcagtgaggcacctatctcagcgatctgtctatttcgttcatccat
agttgcctgactccccgtcgtgtagataactacgatacgggagggcttaccatctggccccagt
gctgcaatgataccgcgagacccacgctcaccggctccagatttatcagcaataaaccagccag
ccggaagggccgagcgcagaagtggtcctgcaactttatccgcctccatccagtctattaattg
ttgccgggaagctagagtaagtagttcgccagttaatagtttgcgcaacgttgttgccattgct
acaggcatcgtggtgtcacgctcgtcgtttggtatggcttcattcagctccggttcccaacgat
caaggcgagttacatgatcccccatgttgtgcaaaaaagcggttagctccttcggtcctccgat
cgttgtcagaagtaagttggccgcagtgttatcactcatggttatggcagcactgcataattct
cttactgtcatgccatccgtaagatgcttttctgtgactggtgagtactcaaccaagtcattct
gagaatagtgtatgcggcgaccgagttgctcttgcccggcgtcaatacgggataataccgcgcc
acatagcagaactttaaaagtgctcatcattggaaaacgttcttcggggcgaaaactctcaagg
atcttaccgctgttgagatccagttcgatgtaacccactcgtgcacccaactgatcttcagcat
cttttactttcaccagcgtttctgggtgagcaaaaacaggaaggcaaaatgccgcaaaaaaggg
aataagggcgacacggaaatgttgaatactcatactcttcctttttcaatattattgaagcatt
tatcagggttattgtctcatgagcggatacatatttgaatgtatttagaaaaataaacaaatag
gggttccgcgcacatttccccgaaaagtgccacctgac

According to some embodiments, p136_Lenti_CBA_tandomarray-Sense-GA80s-GFP-WPRE_1-FP-CBA-01 (1077 bp) comprises SEQ ID NO:81, shown below.

NNNNNNNNNNNNNNNNNNNNANNNGNTCTGCCTTCTTCTTTTTCCTACAG
CTCCTGGGCAACGCCACCATGGCACCCAACTTTTCTATACAAAGTTGTAT
CCTTACTCTAGGACCAAGAATGAACTGCTITCATCTATGAAAGAAGAAAT
AGATGTAAGTTTAAATGAGAGCAATTATACACTTTAATGTATATTATTAA
TATTCTAAACATACTATTCACATACAGTAATAGGAGCAATTAATATTTAA
TGTAGTGTCTTTTGAAACAAAAGAGTGTTAAGAGATACCTTTAGAAGAGG
AAGTTGTTCTTGTAAAAAAAAGTGTTATTTCAACACTATGATACAGTACT
CAATGATGATGATAAAGTAAGAATTTTTCTTTTCATAAAATAGGGACATT
ACGTATTTGAACACTCATTATATTTCTATATATAACAGAATCCTTTCATA
TTAAGTTGTACTGTAGATGAACTTAAGTTATTTAAGCAGTGGAGTTTAGT
ACTTAATATAAGCATTGAGTAAGATAAATAATATAAAAGCTAACATTTCC
TATTTACATTTCTTCTAGACACAGTTACAGATTTTCATGAAATTTTAGCA
TGAGTGTGTTTAACCTAAAGCCTTTCATACATCATTTTAAACATGTCAAT
TTCTTCAGCTACATTAATTAAATGATATTATATTATCTTCAGGTTCCGAA
GAGAACAACTTTGTATAATAAAGTTGTAATGCATCACCACCATCATCACG
ATTATAAGGATGACGATGACAAGGGAGCTGGGGCGGGTGCGGGGGCAGGA
GCCGGAGCCGGCGCGGGCGCNNNGCNGNGCTGGTGCTGGCGCCGGTGCGG
GANCCGGGGCNNCGCTGGGGCGGGCGCTGGTGCTGGTGCTGGTGCCGGGG
CCNGCGCCCGGANCNAGGGCTGGAGCGGGCGCGGGGGCGGGCGCCGNAGC
CGGTGCGGGGGCCGGGGNCGGCGCNNNNCAGCGCTGGCCNCNNNGCTGNA
NCTGGCGCCGGGGCGGGANCAGGGNCNGANAGGCGCTGGTGCCGNNNNNN
GGGCTGGCNCGGGGCAGNTNCAGGNNN

According to some embodiments, p136_Lenti_CBA_tandomarray-Sense-GA80s-GFP-WPRE_1-RP-WPRE-01 (1045 bp) comprises SEQ ID NO: 82, shown below.

NNNNNNNNNNNNNGNNNNNNNNCAGCGTATCCNCATAGCGTAAAAGGAGC
AACATAGTTAAGAATACCAGTCAATCTTTCACAAATTTTGTAATCCAGAG
GTTGATTTCACTTGTACAGCTCGTCCATGCCATGTGTGATCCCAGCAGCG
GTCACAAACTCCAGCAGGACCATGTGGTCTCTCTTTTCGTTGGGATCTTT
AGACAGGGCAGACTGGGTGGACAGGTAATGGTTGTCTGGGAGGAGCACAG
GGCCGTCGCCGATTGGAGTGTTCTGTTGATAATGGTCGGCCAGCTGCACG
GATCCATCCTCAATGTTGTGTCTGATCTTGAAGTTGACCTTGATGCCATT
CTTTTGCTTGTCGGCCATGATGTACACATTGTGGGAGTTATAGTTGTATT
CCAGCTTGTGGCCGAGAATGTTTCCATCCTCCTTAAAGTCAATGCCCTTC
AGCTCGATTCTATTCACCAGGGTGTCACCTTCGAACTTGACTTCAGCGCG
GGTCTTGTAGTTCCCGTCATCTTTGAAAAAGATGGTTCTCTCCTGCACAT
AGCCCTCGGGCATGGCGCTCTTGAAAAAGTCATGCTGCTTCATATGGTCT
GGGTATCTGGAAAAGCACTGCACGCCATAGGTCAGGGTAGTGACCAGTGT
TGGCCATGGCACAGGGAGCTTTCCAGTGGTGCAGATGAATTTCAGGGTGA
GCTTTCCGTATGTGGCATCACCTTCACCCTCTCCGCTGACAGAAAATTTG
TGCCCATTCACATCGCCATCCAGTTCCACGAGAATTGGGACCACGCCAGT
GAACAGTTCCTCGCCCTTGCTCTTGTCATCGTCATCCTTATAATCGTGAT
GATGGTGGTGATGAGCGCCTGCCCCGGCCCCGGCCNCGGCGCCGGCACCG
GNACCCGCGCNGCACCTGCGCCCNCCCTGCCCNANCTCAGCACCGGCACC
AGCCCCGCACTGCGCCNCTCTGCCCNNCCNGCNCNGCACCANNGCNGNNC
NGCCNNNNNNNNTGNNCNGNACNGCCCNNGCNNCCNGNNCNNNAN

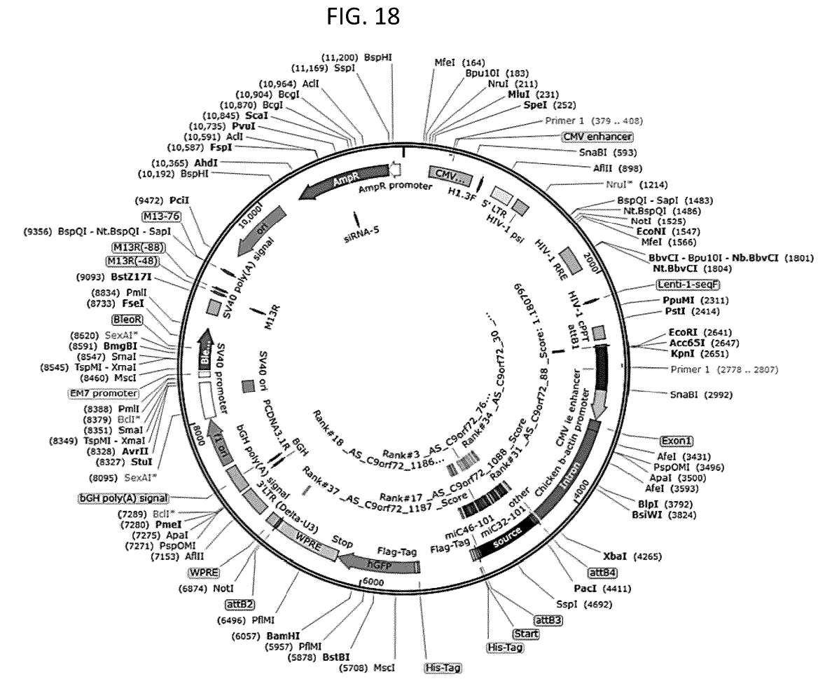

(2) p137_Lenti_CBA_tandomarray-AntiSense-GA80s-GFP-WPRE. This construct comprises CBA promoter, tandomArray-antisense (miRNA targeting site C9orf72 on antisense sequence), Glycine Alanine repeat sequence tagged with GFP gene, WPRE, Ampicillin resistance gene, lentivirus production gene. The vector map is shown in FIG. 18. According to some embodiments, the nucleic acid sequence of p137_Lenti_CBA_tandomarray-AntiSense-GA80s-GFP-WPRE comprises SEQ ID NO: 83. According to some embodiments, the nucleic acid sequence of p137_Lenti_CBA_tandomarray-AntiSense-GA80s-GFP-WPRE is at least 85%, at least 90%, at least 95%, at least 96%, at least 97%, at least 98%, at least 99% identical to SEQ ID NO: 83, shown below.

gtcgacggatcgggagatctcccgatcccctatggtgcactctcagtacaatctgctctgatgc
cgcatagttaagccagtatctgctccctgcttgtgtgttggaggtcgctgagtagtgcgcgagc
aaaatttaagctacaacaaggcaaggcttgaccgacaattgcatgaagaatctgcttagggtta
ggcgttttgcgctgcttcgcgatgtacgggccagatatacgcgttgacattgattattgactag
ttattaatagtaatcaattacggggtcattagttcatagcccatatatggagttccgcgttaca
taacttacggtaaatggcccgcctggctgaccgcccaacgacccccgcccattgacgtcaataa
tgacgtatgttcccatagtaacgccaatagggactttccattgacgtcaatgggtggagtattt
acggtaaactgcccacttggcagtacatcaagtgtatcatatgccaagtacgccccctattgac
gtcaatgacggtaaatggcccgcctggcattatgcccagtacatgaccttatgggactttccta
cttggcagtacatctacgtattagtcatcgctattaccatggtgatgcggttttggcagtacat
caatgggcgtggatagcggtttgactcacggggatttccaagtctccaccccattgacgtcaat
gggagtttgttttggcaccaaaatcaacgggactttccaaaatgtcgtaacaactccgccccat
tgacgcaaatgggcggtaggcgtgtacggtgggaggtctatataagcagcgcgttttgcctgta
ctgggtctctctggttagaccagatctgagcctgggagctctctggctaactagggaacccact
gcttaagcctcaataaagcttgccttgagtgcttcaagtagtgtgtgcccgtctgttgtgtgac
tctggtaactagagatccctcagacccttttagtcagtgtggaaaatctctagcagtggcgccc
gaacagggacttgaaagcgaaagggaaaccagaggagctctctcgacgcaggactcggcttgct
gaagcgcgcacggcaagaggcgaggggcggcgactggtgagtacgccaaaaattttgactagcg
gaggctagaaggagagagatgggtgcgagagcgtcagtattaagcgggggagaattagatcgcg
atgggaaaaaattcggttaaggccagggggaaagaaaaaatataaattaaaacatatagtatgg
gcaagcagggagctagaacgattcgcagttaatcctggcctgttagaaacatcagaaggctgta
gacaaatactgggacagctacaaccatcccttcagacaggatcagaagaacttagatcattata
taatacagtagcaaccctctattgtgtgcatcaaaggatagagataaaagacaccaaggaagct
ttagacaagatagaggaagagcaaaacaaaagtaagaccaccgcacagcaagcggccgctgatc
ttcagacctggaggaggagatatgagggacaattggagaagtgaattatataaatataaagtag
taaaaattgaaccattaggagtagcacccaccaaggcaaagagaagagtggtgcagagagaaaa
aagagcagtgggaataggagctttgttccttgggttcttgggagcagcaggaagcactatgggc
gcagcgtcaatgacgctgacggtacaggccagacaattattgtctggtatagtgcagcagcaga
acaatttgctgagggctattgaggcgcaacagcatctgttgcaactcacagtctggggcatcaa
gcagctccaggcaagaatcctggctgtggaaagatacctaaaggatcaacagctcctggggatt
tggggttgctctggaaaactcatttgcaccactgctgtgccttggaatgctagttggagtaata
aatctctggaacagatttggaatcacacgacctggatggagtgggacagagaaattaacaatta
cacaagcttaatacactccttaattgaagaatcgcaaaaccagcaagaaaagaatgaacaagaa
ttattggaattagataaatgggcaagtttgtggaattggtttaacataacaaattggctgtggt
atataaaattattcataatgatagtaggaggcttggtaggtttaagaatagtttttgctgtact
ttctatagtgaatagagttaggcagggatattcaccattatcgtttcagacccacctcccaacc
ccgaggggacccgacaggcccgaaggaatagaagaagaaggtggagagagagacagagacagat
ccattcgattagtgaacggatcggcactgcgtgcgccaattctgcagacaaatggcagtattca
tccacaattttaaaagaaaaggggggattggggggtacagtgcaggggaaagaatagtagacat
aatagcaacagacatacaaactaaagaattacaaaaacaaattacaaaaattcaaaattttcgg
gtttattacagggacagcagagatccagtttggttaatggCCGCacaagtttGTACAAAAAAGC
AGGCTTActcagatctgaattcggtacctagttattaatagtaatcaattacggggtcattagt
tcatagcccatatatggagttccgcgttacataacttacggtaaatggcccgcctggctgaccg
cccaacgacccccgcccattgacgtcaataatgacgtatgttcccatagtaacgccaataggga
ctttccattgacgtcaatgggtggagtatttacggtaaactgcccacttggcagtacatcaagt
gtatcatatgccaagtacgccccctattgacgtcaatgacggtaaatggcccgcctggcattat
gcccagtacatgaccttatgggactttcctacttggcagtacatctacgtattagtcatcgcta
ttaccatggtcgaggtgagccccacgttctgcttcactctccccatctcccccccctccccacc
cccaattttgtatttatttattttttaattattttgtgcagcgatgggggcggggggggggggg
gggcgcgcgccaggcggggcggggcggggcgaggggcggggcggggcgaggcggagaggtgcgg
cggcagccaatcagagcggcgcgctccgaaagtttccttttatggcgaggcggcggcggcggcg
gccctataaaaagcgaagcgcgcggcgggcgggagtcgctgcgcgctgccttcgccccgtgccc
cgctccgccgccgcctcgcgccgcccgccccggctctgactgaccgcgttactcccacaggtga
gcgggcgggacggcccttctcctccgggctgtaattagcgcttggtttaatgacggcttgtttc
ttttctgtggctgcgtgaaagccttgaggggctccgggagggccctttgtgcggggggagcggc
tcggggggtgcgtgcgtgtgtgtgtgcgtggggagcgccgcgtgcggctccgcgctgcccggcg
gctgtgagcgctgcgggcgcggcgcggggctttgtgcgctccgcagtgtgcgcgaggggagcgc
ggccgggggcggtgccccgcggtgcggggggggctgcgaggggaacaaaggctgcgtgcggggt
gtgtgcgtgggggggtgagcagggggtgtgggcgcgtcggtcgggctgcaaccccccctgcacc
cccctccccgagttgctgagcacggcccggcttcgggtgcggggctccgtacggggcgtggcgc
ggggctcgccgtgccgggcggggggtggcggcaggtgggggtgccgggcggggcggggccgcct
cgggccggggagggctcgggggaggggcgcggcggcccccggagcgccggcggctgtcgaggcg
cggcgagccgcagccattgccttttatggtaatcgtgcgagagggcgcagggacttcctttgtc
ccaaatctgtgcggagccgaaatctgggaggcgccgccgcaccccctctagcgggcgcggggcg
aagcggtgcggcgccggcaggaaggaaatgggcggggagggccttcgtgcgtcgccgcgccgcc
gtccccttctccctctccagcctcggggctgtccgcggggggacggctgccttcgggggggacg
gggcagggcggggttcggcttctggcgtgtgaccggcggctctagagcctctgctaaccatgtt
catgccttcttctttttcctacagctcctgggcaacgccaccatggCACCCAACTTTTCTATAC
AAAGTTGTATCCTTACTCTAGGACCAAGAATCCATACATGCAGACATGATTACATTAATTAACA
TGAGGTTTTGCTTTTTCTTTAATCCCTGATTGGTATTTAGAAACCACTGCTATTGTAGTGAAAA
TTCTACAATCATAAAGCCCTCACTTCTTGTTTTTTACCCGGCTAAGTTTTTAATTTTTCCTGGC
TCTCAATACTTGTAAGACAGTGAACTGTTTACAGTACCAGAAAGTTCACAACACTTTCTCAATC
TTCAATGGAAGGTGAAGTTCATATCACTATCCTGGGAACTATCTAATTAACGTAGAATAGAATG
CCAACATAGCCAAACAAAATATTTTATCAACTCGTTCTTGTTTCAGATGTATAGCAGTTTCCAA
CTGATTCAACCGTATTTCAAGTATTCTGAGATAGTCTTGTTTCTGTGATATTCACAGATTATGT
TAAAAGTTTCTCTGAGAAAAATCATATCTTAATGCATGGCAACTGTTTGAATAGAAATTTACCC
CCTCCTGTTTCTGAATACAAATCTGTGCACTTCTTTAGACAATCCTTGTTTTCTTCTGGTTAAT
TATCTTCAGGTTCCGAAGAGAACAACTTTGTATAATAAAGTTGTAATGCATCACCACCATCATC
ACGATTATAAGGATGACGATGACAAGGGAGCTGGGGCGGGTGCGGGGGCAGGAGCCGGAGCCGG
CGCGGGCGCAGGTGCAGGTGCTGGTGCTGGCGCCGGTGCGGGAGCCGGGGCAGGCGCTGGGGCG
GGCGCTGGTGCTGGTGCTGGTGCCGGGGCCGGCGCCGGAGCAGGGGCTGGAGCGGGCGCGGGGG
CGGGCGCCGGAGCCGGTGCGGGGGCCGGGGCCGGCGCAGGCGCAGGCGCTGGCGCCGGTGCTGG
AGCTGGCGCCGGGGCGGGAGCAGGGGCCGGAGCAGGCGCTGGTGCCGGCGCAGGGGCTGGCGCG
GGGGCAGGTGCAGGCGCAGGTGCCGGTGCCGGGGCAGGCGCTGGCGCTGGTGCCGGCGCAGGGG
CAGGGGCAGGAGCGGGCGCAGGTGCGGGGGCTGGTGCCGGTGCTGGAGCTGGGGCAGGGGCGGG
CGCAGGTGCCGGCGCGGGTGCCGGTGCCGGCGCCGGGGCCGGGGCCGGGGCAGGCGCTCATCAC
CACCATCATCACGATTATAAGGATGACGATGACAAGagcaagggcgaggaactgttcactggcg
tggtcccaattctcgtggaactggatggcgatgtgaatgggcacaaattttctgtcagcggaga
gggtgaaggtgatgccacatacggaaagctcaccctgaaattcatctgcaccactggaaagctc
cctgtgccatggccaacactggtcactaccctgacctatggcgtgcagtgcttttccagatacc
cagaccatatgaagcagcatgactttttcaagagcgccatgcccgagggctatgtgcaggagag
aaccatctttttcaaagatgacgggaactacaagacccgcgctgaagtcaagttcgaaggtgac
accctggtgaatagaatcgagctgaagggcattgactttaaggaggatggaaacattctcggcc
acaagctggaatacaactataactcccacaatgtgtacatcatggccgacaagcaaaagaatgg
catcaaggtcaacttcaagatcagacacaacattgaggatggatccgtgcagctggccgaccat
tatcaacagaacactccaatcggcgacggccctgtgctcctcccagacaaccattacctgtcca
cccagtctgccctgtctaaagatcccaacgaaaagagagaccacatggtcctgctggagtttgt
gaccgctgctgggatcacacatggcatggacgagctgtacaagTGAaatcaacctctggattac
aaaatttgtgaaagattgactggtattcttaactatgttgctccttttacgctatgtggatacg
ctgctttaatgcctttgtatcatgctattgcttcccgtatggctttcattttctcctccttgta
taaatcctggttgctgtctctttatgaggagttgtggcccgttgtcaggcaacgtggcgtggtg
tgcactgtgtttgctgacgcaacccccactggttggggcattgccaccacctgtcagctccttt
ccgggactttcgctttccccctccctattgccacggcggaactcatcgccgcctgccttgcccg
ctgctggacaggggctcggctgttgggcactgacaattccgtggtgttgtcggggaaatcatcg
tcctttccttggctgctcgcctgtgttgccacctggattctgcgcgggacgtccttctgctacg
tcccttcggccctcaatccagcggaccttccttcccgcggcctgctgccggctctgcggcctct
tccgcgtcttcgccttcgccctcagacgagtcggatctccctttgggccgcctccccgcctgAA
CCCAGCTTTcttgtacaaagtggtGCGGccgcggcctgctgccggctctgcggcctcttccgcg
tcttcgccttcgccctcagacgagtcggatctccctttgggccgcctccccgcgtcgactttaa
gaccaatgacttacaaggcagctgtagatcttagccactttttaaaagaaaaggggggactgga
agggctaattcactcccaacgaagacaagatctgctttttgcttgtactgggtctctctggtta
gaccagatctgagcctgggagctctctggctaactagggaacccactgcttaagcctcaataaa
gcttgccttgagtgcttcaagtagtgtgtgcccgtctgttgtgtgactctggtaactagagatc
cctcagacccttttagtcagtgtggaaaatctctagcagggcccgtttaaacccgctgatcagc
ctcgactgtgccttctagttgccagccatctgttgtttgcccctcccccgtgccttccttgacc
ctggaaggtgccactcccactgtcctttcctaataaaatgaggaaattgcatcgcattgtctga
gtaggtgtcattctattctggggggtggggtggggcaggacagcaagggggaggattgggaaga
caatagcaggcatgctggggatgcggtgggctctatggcttctgaggcggaaagaaccagctgg
ggctctagggggtatccccacgcgccctgtagcggcgcattaagcgcggcgggtgtggtggtta
cgcgcagcgtgaccgctacacttgccagcgccctagcgcccgctcctttcgctttcttcccttc
ctttctcgccacgttcgccggctttccccgtcaagctctaaatcgggggctccctttagggttc
cgatttagtgctttacggcacctcgaccccaaaaaacttgattagggtgatggttcacgtagtg
ggccatcgccctgatagacggtttttcgccctttgacgttggagtccacgttctttaatagtgg
actcttgttccaaactggaacaacactcaaccctatctcggtctattcttttgatttataaggg
attttgccgatttcggcctattggttaaaaaatgagctgatttaacaaaaatttaacgcgaatt
aattctgtggaatgtgtgtcagttagggtgtggaaagtccccaggctccccagcaggcagaagt
atgcaaagcatgcatctcaattagtcagcaaccaggtgtggaaagtccccaggctccccagcag
gcagaagtatgcaaagcatgcatctcaattagtcagcaaccatagtcccgcccctaactccgcc
catcccgcccctaactccgcccagttccgcccattctccgccccatggctgactaatttttttt
atttatgcagaggccgaggccgcctctgcctctgagctattccagaagtagtgaggaggctttt
ttggaggcctaggcttttgcaaaaagctcccgggagcttgtatatccattttcggatctgatca
gcacgtgttgacaattaatcatcggcatagtatatcggcatagtataatacgacaaggtgagga
actaaaccatggccaagttgaccagtgccgttccggtgctcaccgcgcgcgacgtcgccggagc
ggtcgagttctggaccgaccggctcgggttctcccgggacttcgtggaggacgacttcgccggt
gtggtccgggacgacgtgaccctgttcatcagcgcggtccaggaccaggtggtgccggacaaca
ccctggcctgggtgtgggtgcgcggcctggacgagctgtacgccgagtggtcggaggtcgtgtc
cacgaacttccgggacgcctccgggccggccatgaccgagatcggcgagcagccgtgggggcgg
gagttcgccctgcgcgacccggccggcaactgcgtgcacttcgtggccgaggagcaggactgac
acgtgctacgagatttcgattccaccgccgccttctatgaaaggttgggcttcggaatcgtttt
ccgggacgccggctggatgatcctccagcgcggggatctcatgctggagttcttcgcccacccc
aacttgtttattgcagcttataatggttacaaataaagcaatagcatcacaaatttcacaaata
aagcatttttttcactgcattctagttgtggtttgtccaaactcatcaatgtatcttatcatgt
ctgtataccgtcgacctctagctagagcttggcgtaatcatggtcatagctgtttcctgtgtga
aattgttatccgctcacaattccacacaacatacgagccggaagcataaagtgtaaagcctggg
gtgcctaatgagtgagctaactcacattaattgcgttgcgctcactgcccgctttccagtcggg
aaacctgtcgtgccagctgcattaatgaatcggccaacgcgcggggagaggcggtttgcgtatt
gggcgctcttccgcttcctcgctcactgactcgctgcgctcggtcgttcggctgcggcgagcgg
tatcagctcactcaaaggcggtaatacggttatccacagaatcaggggataacgcaggaaagaa
catgtgagcaaaaggccagcaaaaggccaggaaccgtaaaaaggccgcgttgctggcgtttttc
cataggctccgcccccctgacgagcatcacaaaaatcgacgctcaagtcagaggtggcgaaacc
cgacaggactataaagataccaggcgtttccccctggaagctccctcgtgcgctctcctgttcc
gaccctgccgcttaccggatacctgtccgcctttctcccttcgggaagcgtggcgctttctcat
agctcacgctgtaggtatctcagttcggtgtaggtcgttcgctccaagctgggctgtgtgcacg
aaccccccgttcagcccgaccgctgcgccttatccggtaactatcgtcttgagtccaacccggt
aagacacgacttatcgccactggcagcagccactggtaacaggattagcagagcgaggtatgta
ggcggtgctacagagttcttgaagtggtggcctaactacggctacactagaagaacagtatttg
gtatctgcgctctgctgaagccagttaccttcggaaaaagagttggtagctcttgatccggcaa
acaaaccaccgctggtagcggtggtttttttgtttgcaagcagcagattacgcgcagaaaaaaa
ggatctcaagaagatcctttgatcttttctacggggtctgacgctcagtggaacgaaaactcac
gttaagggattttggtcatgagattatcaaaaaggatcttcacctagatccttttaaattaaaa
atgaagttttaaatcaatctaaagtatatatgagtaaacttggtctgacagttaccaatgctta
atcagtgaggcacctatctcagcgatctgtctatttcgttcatccatagttgcctgactccccg
tcgtgtagataactacgatacgggagggcttaccatctggccccagtgctgcaatgataccgcg
agacccacgctcaccggctccagatttatcagcaataaaccagccagccggaagggccgagcgc
agaagtggtcctgcaactttatccgcctccatccagtctattaattgttgccgggaagctagag
taagtagttcgccagttaatagtttgcgcaacgttgttgccattgctacaggcatcgtggtgtc
acgctcgtcgtttggtatggcttcattcagctccggttcccaacgatcaaggcgagttacatga
tcccccatgttgtgcaaaaaagcggttagctccttcggtcctccgatcgttgtcagaagtaagt
tggccgcagtgttatcactcatggttatggcagcactgcataattctcttactgtcatgccatc
cgtaagatgcttttctgtgactggtgagtactcaaccaagtcattctgagaatagtgtatgcgg
cgaccgagttgctcttgcccggcgtcaatacgggataataccgcgccacatagcagaactttaa
aagtgctcatcattggaaaacgttcttcggggcgaaaactctcaaggatcttaccgctgttgag
atccagttcgatgtaacccactcgtgcacccaactgatcttcagcatcttttactttcaccagc
gtttctgggtgagcaaaaacaggaaggcaaaatgccgcaaaaaagggaataagggcgacacgga
aatgttgaatactcatactcttcctttttcaatattattgaagcatttatcagggttattgtct
catgagcggatacatatttgaatgtatttagaaaaataaacaaataggggttccgcgcacattt
ccccgaaaagtgccacctgac

According to some embodiments, p137_Lenti_CBA_tandomarray-AntiSense-GA80s-GFP-WPRE_6-FP-CBA-01 (1028 bp) comprises SEQ ID NO: 84, shown below.

NNNNNNNNNNNNCNCNGCNNNNTGTTNNTGCCTTCTTCTTTTTCCTACAG
CTCCTGGGCAACGCCACCATGGCACCCAACTTTTCTATACAAAGTTGTAT
CCTTACTCTAGGACCAAGAATCCATACATGCAGACATGATTACATTAATT
AACATGAGGTTTTGCTTTTTCTTTAATCCCTGATTGGTATTTAGAAACCA
CTGCTATTGTAGTGAAAATTCTACAATCATAAAGCCCTCACTTCTTGTTT
TTTACCCGGCTAAGTTTTTAATTTTTCCTGGCTCTCAATACTTGTAAGAC
AGTGAACTGTTTACAGTACCAGAAAGTTCACAACACTTTCTCAATCTTCA
ATGGAAGGTGAAGTTCATATCACTATCCTGGGAACTATCTAATTAACGTA
GAATAGAATGCCAACATAGCCAAACAAAATATTTTATCAACTCGTTCTTG
TTTCAGATGTATAGCAGTTTCCAACTGATTCAACCGTATTTCAAGTATTC
TGAGATAGTCTTGTTTCTGTGATATTCACAGATTATGTTAAAAGTTTCTC
TGAGAAAAATCATATCTTAATGCATGGCAACTGTTTGAATAGAAATTTAC
CCCCTCCTGTTTCTGAATACAAATCTGTGCACTTCTTTAGACAATCCTTG
TTTTCTTCTGGTTAATTATCTTCAGGTTCCGAAGAGAACAACTTTGTATA
ATAAAGTTGTAATGCATCACCACCATCATCACGATTATAAGGATGACGAT
GACAAGGGAGCTGGGGCGGGTGCNGGGGGCANGAGCCGGANCCGGCGCGG
GCGCANGTGCAGGTGCTGGTGCTGGCGCCGGTGCGGGAGCCGGGGCNGCG
CTGGGGCGGGCGCTGGTGCTGGTGCTGGTGCCGGGGCCGGCGCCGGANCA
GGGCTGGAGCGGGCGCGGGGCGGGCGCCGGANCCGGTGCGGGGGCCGGGG
CCGGCGCNNCGCNGCGCTGGCGCCGGTGCTGGANCTGGCNCCCGGGNCGG
GANCAGGGNNNGGNANCNGGCNCTGGNN

According to some embodiments, p137_Lenti_CBA_tandomarray-AntiSense-GA80s-GFP-WPRE_6-RP-WPRE-01 (1033 bp) comprises SEQ ID NO: 85, shown below.

NNNNNNNNNNNNNNGNNNNTANNNCAGCGTATCCACATAGCGTAAAAGGA
GCAACATAGTTAAGAATACCAGTCAATCTTTCACAAATTTTGTAATCCAG
AGGTTGATTTCACTTGTACAGCTCGTCCATGCCATGTGTGATCCCAGCAG
CGGTCACAAACTCCAGCAGGACCATGTGGTCTCTCTTTTCGTTGGGATCT
TTAGACAGGGCAGACTGGGTGGACAGGTAATGGTTGTCTGGGAGGAGCAC
AGGGCCGTCGCCGATTGGAGTGTTCTGTTGATAATGGTCGGCCAGCTGCA
CGGATCCATCCTCAATGTTGTGTCTGATCTTGAAGTTGACCTTGATGCCA
TTCTTTTGCTTGTCGGCCATGATGTACACATTGTGGGAGTTATAGTTGTA
TTCCAGCTTGTGGCCGAGAATGTTTCCATCCTCCTTAAAGTCAATGCCCT
TCAGCTCGATTCTATTCACCAGGGTGTCACCTTCGAACTTGACTTCAGCG
CGGGTCTTGTAGTTCCCGTCATCTTTGAAAAAGATGGTTCTCTCCTGCAC
ATAGCCCTCGGGCATGGCGCTCTTGAAAAAGTCATGCTGCTTCATATGGT
CTGGGTATCTGGAAAAGCACTGCACGCCATAGGTCAGGGTAGTGACCAGT
GTTGGCCATGGCACAGGGAGCTTTCCAGTGGTGCAGATGAATTTCAGGGT
GAGCTTTCCGTATGTGGCATCACCTTCACCCTCTCCGCTGACANNAAAAT
TTGTGCCCATTCACATCGCCATCCAGTTCCNCGAGAATTGGGACCACGCC
AGTGAACAGTTCCTCGCCCTTGCTCTTGTCATCGTCATCCTTATAATCGT
GATGATGGTGGTGATGAGCGCCTGCCCCGGCCCCGGCCCCGGCGCCGGCA
CCGGCACCCCGCGCCGGGNANCTGCGCCCGCCCCNGCCCCAACTTCAGCA
NCNGCACCANCCCCGNNNCNTGNCCCCNCTNCCTGCCCCNNGCCCCTGCG
CCGAGNACCAACGNCANGNGCTCTGNCCCNNNN

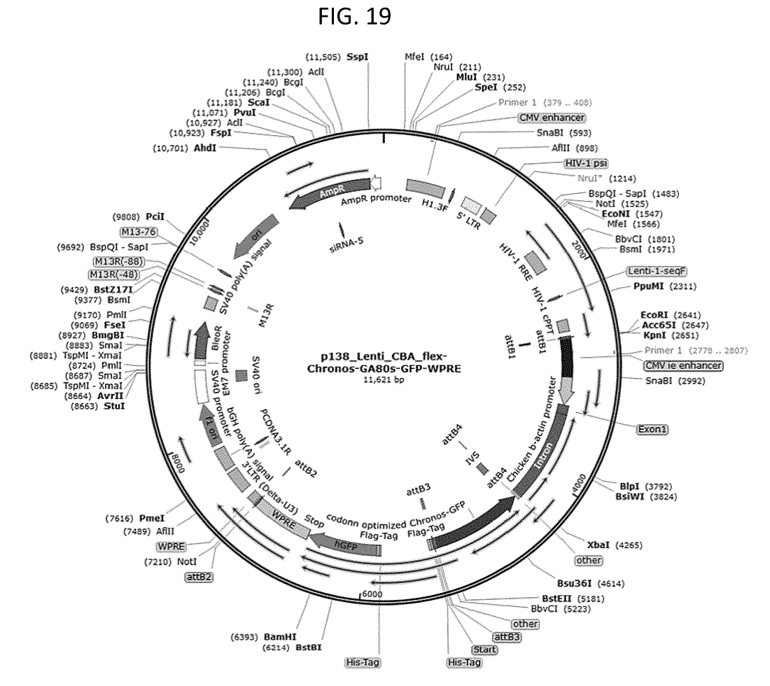

(3) p138_Lenti_CBA_flex-Chronos-GA80s-GFP-WPRE. This construct comprises CBA promoter, partial of Chronos GFP sequence, Glycine Alanine repeat sequence tagged with GFP gene, WPRE, Ampicillin resistance gene, lentivirus production gene. The vector map is shown in FIG. 19. According to some embodiments, the nucleic acid sequence of p138_Lenti_CBA_flex-Chronos-GA80s-GFP-WPRE comprises SEQ ID NO: 86. According to some embodiments, the nucleic acid sequence of p138_Lenti_CBA_flex-Chronos-GA80s-GFP-WPRE is at least 85%, at least 90%, at least 95%, at least 96%, at least 97%, at least 98%, at least 99% identical to SEQ ID NO: 86, shown below.

gtcgacggatcgggagatctcccgatcccctatggtgcactctcagtacaatctgctctgatgc
cgcatagttaagccagtatctgctccctgcttgtgtgttggaggtcgctgagtagtgcgcgagc
aaaatttaagctacaacaaggcaaggcttgaccgacaattgcatgaagaatctgcttagggtta
ggcgttttgcgctgcttcgcgatgtacgggccagatatacgcgttgacattgattattgactag
ttattaatagtaatcaattacggggtcattagttcatagcccatatatggagttccgcgttaca
taacttacggtaaatggcccgcctggctgaccgcccaacgacccccgcccattgacgtcaataa
tgacgtatgttcccatagtaacgccaatagggactttccattgacgtcaatgggtggagtattt
acggtaaactgcccacttggcagtacatcaagtgtatcatatgccaagtacgccccctattgac
gtcaatgacggtaaatggcccgcctggcattatgcccagtacatgaccttatgggactttccta
cttggcagtacatctacgtattagtcatcgctattaccatggtgatgcggttttggcagtacat
caatgggcgtggatagcggtttgactcacggggatttccaagtctccaccccattgacgtcaat
gggagtttgttttggcaccaaaatcaacgggactttccaaaatgtcgtaacaactccgccccat
tgacgcaaatgggcggtaggcgtgtacggtgggaggtctatataagcagcgcgttttgcctgta
ctgggtctctctggttagaccagatctgagcctgggagctctctggctaactagggaacccact
gcttaagcctcaataaagcttgccttgagtgcttcaagtagtgtgtgcccgtctgttgtgtgac
tctggtaactagagatccctcagacccttttagtcagtgtggaaaatctctagcagtggcgccc
gaacagggacttgaaagcgaaagggaaaccagaggagctctctcgacgcaggactcggcttgct
gaagcgcgcacggcaagaggcgaggggcggcgactggtgagtacgccaaaaattttgactagcg
gaggctagaaggagagagatgggtgcgagagcgtcagtattaagcgggggagaattagatcgcg
atgggaaaaaattcggttaaggccagggggaaagaaaaaatataaattaaaacatatagtatgg
gcaagcagggagctagaacgattcgcagttaatcctggcctgttagaaacatcagaaggctgta
gacaaatactgggacagctacaaccatcccttcagacaggatcagaagaacttagatcattata
taatacagtagcaaccctctattgtgtgcatcaaaggatagagataaaagacaccaaggaagct
ttagacaagatagaggaagagcaaaacaaaagtaagaccaccgcacagcaagcggccgctgatc
ttcagacctggaggaggagatatgagggacaattggagaagtgaattatataaatataaagtag
taaaaattgaaccattaggagtagcacccaccaaggcaaagagaagagtggtgcagagagaaaa
aagagcagtgggaataggagctttgttccttgggttcttgggagcagcaggaagcactatgggc
gcagcgtcaatgacgctgacggtacaggccagacaattattgtctggtatagtgcagcagcaga
acaatttgctgagggctattgaggcgcaacagcatctgttgcaactcacagtctggggcatcaa
gcagctccaggcaagaatcctggctgtggaaagatacctaaaggatcaacagctcctggggatt
tggggttgctctggaaaactcatttgcaccactgctgtgccttggaatgctagttggagtaata
aatctctggaacagatttggaatcacacgacctggatggagtgggacagagaaattaacaatta
cacaagcttaatacactccttaattgaagaatcgcaaaaccagcaagaaaagaatgaacaagaa
ttattggaattagataaatgggcaagtttgtggaattggtttaacataacaaattggctgtggt
atataaaattattcataatgatagtaggaggcttggtaggtttaagaatagtttttgctgtact
ttctatagtgaatagagttaggcagggatattcaccattatcgtttcagacccacctcccaacc
ccgaggggacccgacaggcccgaaggaatagaagaagaaggtggagagagagacagagacagat
ccattcgattagtgaacggatcggcactgcgtgcgccaattctgcagacaaatggcagtattca
tccacaattttaaaagaaaaggggggattggggggtacagtgcaggggaaagaatagtagacat
aatagcaacagacatacaaactaaagaattacaaaaacaaattacaaaaattcaaaattttcgg
gtttattacagggacagcagagatccagtttggttaatggCCGCacaagtttGTACAAAAAAGC
AGGCTTActcagatctgaattcggtacctagttattaatagtaatcaattacggggtcattagt
tcatagcccatatatggagttccgcgttacataacttacggtaaatggcccgcctggctgaccg
cccaacgacccccgcccattgacgtcaataatgacgtatgttcccatagtaacgccaataggga
ctttccattgacgtcaatgggtggagtatttacggtaaactgcccacttggcagtacatcaagt
gtatcatatgccaagtacgccccctattgacgtcaatgacggtaaatggcccgcctggcattat
gcccagtacatgaccttatgggactttcctacttggcagtacatctacgtattagtcatcgcta
ttaccatggtcgaggtgagccccacgttctgcttcactctccccatctcccccccctccccacc
cccaattttgtatttatttattttttaattattttgtgcagcgatgggggcggggggggggggg
gggcgcgcgccaggcggggcggggcggggcgaggggcggggcggggcgaggcggagaggtgcgg
cggcagccaatcagagcggcgcgctccgaaagtttccttttatggcgaggcggcggcggcggcg
gccctataaaaagcgaagcgcgcggcgggcgggagtcgctgcgcgctgccttcgccccgtgccc
cgctccgccgccgcctcgcgccgcccgccccggctctgactgaccgcgttactcccacaggtga
gcgggcgggacggcccttctcctccgggctgtaattagcgcttggtttaatgacggcttgtttc
ttttctgtggctgcgtgaaagccttgaggggctccgggagggccctttgtgcggggggagcggc
tcggggggtgcgtgcgtgtgtgtgtgcgtggggagcgccgcgtgcggctccgcgctgcccggcg
gctgtgagcgctgcgggcgcggcgcggggctttgtgcgctccgcagtgtgcgcgaggggagcgc
ggccgggggcggtgccccgcggtgcggggggggctgcgaggggaacaaaggctgcgtgcggggt
gtgtgcgtgggggggtgagcagggggtgtgggcgcgtcggtcgggctgcaaccccccctgcacc
cccctccccgagttgctgagcacggcccggcttcgggtgcggggctccgtacggggcgtggcgc
ggggctcgccgtgccgggcggggggtggcggcaggtgggggtgccgggcggggcggggccgcct
cgggccggggagggctcgggggaggggcgcggcggcccccggagcgccggcggctgtcgaggcg
cggcgagccgcagccattgccttttatggtaatcgtgcgagagggcgcagggacttcctttgtc
ccaaatctgtgcggagccgaaatctgggaggcgccgccgcaccccctctagcgggcgcggggcg
aagcggtgcggcgccggcaggaaggaaatgggcggggagggccttcgtgcgtcgccgcgccgcc
gtccccttctccctctccagcctcggggctgtccgcggggggacggctgccttcgggggggacg
gggcagggcggggttcggcttctggcgtgtgaccggcggctctagagcctctgctaaccatgtt
catgccttcttctttttcctacagctcctgggcaacgccaccatggCACCCAACTTTTCTATAC
AAAGTTGTAtctctgtctcgacaagcccagtttctattggtctccttaaacctgtcttgtaacc
ttgatacttacCAGGTGGTGGCCCAGGAAGCCCCAGGTGTTTTTGCTTATCAGATCCAGGATCA
GATGGCCGATGCCGCTGGTGTATGGGGTGATCAGGCCGAGGCCCTCGTGTCCGGCAATGAACAT
CACGGGGAACATCAGCCAGCTGCAGAAAAAGACGTAGGCCATGATTTTACAGATCTTTCTGCAC
ACGCCCTTAGGCAGTGTGTGGTAGCTTTCGATGTACACCTTGGCGATCTGAAAGAAGCATGTGA
CGCCGTAAAAGAGTCCGATCATGAAGAACAGAATTTTCAGAGGGCCCTTGGTAAAAGCGGCGGT
GATTCCCCACACGATGTTGCCGATGTCTGTCACGAGGATTGTCATGGTTCTCTTGCTGTACTCC
TCGTGCAGTCCAGTCAGGTTGCTCAGGTGGATCAGGATAACGGGGCAGGTCAGCAGCCACATGG
AGTACCGCAGCCAGATCACGGCGCCGCCGTTGGTCTGATACACGGTGGCAGGGCTGTCCACTTC
GTGAAACAGCTCGATAAAGCACTTCACCAGCTCAATCACACACACGTACACTTCCTCCCAGCCG
GTTGTGGCCTTGAATGAGTGCCAGCCGTAGAAGATCAGCTGCACGATGGCCACAATCACTGTGA
ACCACTGCAGGCCCACGGCGATCTTGTGCTGCAGCTCGGTGCCGTGGTTAATGTGAGGAAAACA
ACCATGATCGGCGCCGGCTGTTGTGGCATTAGATGTCTCGCCGTGGGCGTCGGCAGCAGGGGTC
ACCACGGCGGCGGCAGACAGCAGGCCCCTGATTGTGGCCTCAGCAGATGGCACAGCGCTTATGA
AGGCGTGGGTCATGGTGGCGGCTGTTTCCATGGTGGCACAACTTTGTATAATAAAGTTGTAATG
CATCACCACCATCATCACGATTATAAGGATGACGATGACAAGGGAGCTGGGGCGGGTGCGGGGG
CAGGAGCCGGAGCCGGCGCGGGCGCAGGTGCAGGTGCTGGTGCTGGCGCCGGTGCGGGAGCCGG
GGCAGGCGCTGGGGCGGGCGCTGGTGCTGGTGCTGGTGCCGGGGCCGGCGCCGGAGCAGGGGCT
GGAGCGGGCGCGGGGGCGGGCGCCGGAGCCGGTGCGGGGGCCGGGGCCGGCGCAGGCGCAGGCG
CTGGCGCCGGTGCTGGAGCTGGCGCCGGGGCGGGAGCAGGGGCCGGAGCAGGCGCTGGTGCCGG
CGCAGGGGCTGGCGCGGGGGCAGGTGCAGGCGCAGGTGCCGGTGCCGGGGCAGGCGCTGGCGCT
GGTGCCGGCGCAGGGGCAGGGGCAGGAGCGGGCGCAGGTGCGGGGGCTGGTGCCGGTGCTGGAG
CTGGGGCAGGGGCGGGCGCAGGTGCCGGCGCGGGTGCCGGTGCCGGCGCCGGGGCCGGGGCCGG
GGCAGGCGCTCATCACCACCATCATCACGATTATAAGGATGACGATGACAAGagcaagggcgag
gaactgttcactggcgtggtcccaattctcgtggaactggatggcgatgtgaatgggcacaaat
tttctgtcagcggagagggtgaaggtgatgccacatacggaaagctcaccctgaaattcatctg
caccactggaaagctccctgtgccatggccaacactggtcactaccctgacctatggcgtgcag
tgcttttccagatacccagaccatatgaagcagcatgactttttcaagagcgccatgcccgagg
gctatgtgcaggagagaaccatctttttcaaagatgacgggaactacaagacccgcgctgaagt
caagttcgaaggtgacaccctggtgaatagaatcgagctgaagggcattgactttaaggaggat
ggaaacattctcggccacaagctggaatacaactataactcccacaatgtgtacatcatggccg
acaagcaaaagaatggcatcaaggtcaacttcaagatcagacacaacattgaggatggatccgt
gcagctggccgaccattatcaacagaacactccaatcggcgacggccctgtgctcctcccagac
aaccattacctgtccacccagtctgccctgtctaaagatcccaacgaaaagagagaccacatgg
tcctgctggagtttgtgaccgctgctgggatcacacatggcatggacgagctgtacaagTGAaa
tcaacctctggattacaaaatttgtgaaagattgactggtattcttaactatgttgctcctttt
acgctatgtggatacgctgctttaatgcctttgtatcatgctattgcttcccgtatggctttca
ttttctcctccttgtataaatcctggttgctgtctctttatgaggagttgtggcccgttgtcag
gcaacgtggcgtggtgtgcactgtgtttgctgacgcaacccccactggttggggcattgccacc
acctgtcagctcctttccgggactttcgctttccccctccctattgccacggcggaactcatcg
ccgcctgccttgcccgctgctggacaggggctcggctgttgggcactgacaattccgtggtgtt
gtcggggaaatcatcgtcctttccttggctgctcgcctgtgttgccacctggattctgcgcggg
acgtccttctgctacgtcccttcggccctcaatccagcggaccttccttcccgcggcctgctgc
cggctctgcggcctcttccgcgtcttcgccttcgccctcagacgagtcggatctccctttgggc
cgcctccccgcctgAACCCAGCTTTcttgtacaaagtggtGCGGccgcggcctgctgccggctc
tgcggcctcttccgcgtcttcgccttcgccctcagacgagtcggatctccctttgggccgcctc
cccgcgtcgactttaagaccaatgacttacaaggcagctgtagatcttagccactttttaaaag
aaaaggggggactggaagggctaattcactcccaacgaagacaagatctgctttttgcttgtac
tgggtctctctggttagaccagatctgagcctgggagctctctggctaactagggaacccactg
cttaagcctcaataaagcttgccttgagtgcttcaagtagtgtgtgcccgtctgttgtgtgact
ctggtaactagagatccctcagacccttttagtcagtgtggaaaatctctagcagggcccgttt
aaacccgctgatcagcctcgactgtgccttctagttgccagccatctgttgtttgcccctcccc
cgtgccttccttgaccctggaaggtgccactcccactgtcctttcctaataaaatgaggaaatt
gcatcgcattgtctgagtaggtgtcattctattctggggggtggggtggggcaggacagcaagg
gggaggattgggaagacaatagcaggcatgctggggatgcggtgggctctatggcttctgaggc
ggaaagaaccagctggggctctagggggtatccccacgcgccctgtagcggcgcattaagcgcg
gcgggtgtggtggttacgcgcagcgtgaccgctacacttgccagcgccctagcgcccgctcctt
tcgctttcttcccttcctttctcgccacgttcgccggctttccccgtcaagctctaaatcgggg
gctccctttagggttccgatttagtgctttacggcacctcgaccccaaaaaacttgattagggt
gatggttcacgtagtgggccatcgccctgatagacggtttttcgccctttgacgttggagtcca
cgttctttaatagtggactcttgttccaaactggaacaacactcaaccctatctcggtctattc
ttttgatttataagggattttgccgatttcggcctattggttaaaaaatgagctgatttaacaa
aaatttaacgcgaattaattctgtggaatgtgtgtcagttagggtgtggaaagtccccaggctc
cccagcaggcagaagtatgcaaagcatgcatctcaattagtcagcaaccaggtgtggaaagtcc
ccaggctccccagcaggcagaagtatgcaaagcatgcatctcaattagtcagcaaccatagtcc
cgcccctaactccgcccatcccgcccctaactccgcccagttccgcccattctccgccccatgg
ctgactaattttttttatttatgcagaggccgaggccgcctctgcctctgagctattccagaag
tagtgaggaggcttttttggaggcctaggcttttgcaaaaagctcccgggagcttgtatatcca
ttttcggatctgatcagcacgtgttgacaattaatcatcggcatagtatatcggcatagtataa
tacgacaaggtgaggaactaaaccatggccaagttgaccagtgccgttccggtgctcaccgcgc
gcgacgtcgccggagcggtcgagttctggaccgaccggctcgggttctcccgggacttcgtgga
ggacgacttcgccggtgtggtccgggacgacgtgaccctgttcatcagcgcggtccaggaccag
gtggtgccggacaacaccctggcctgggtgtgggtgcgcggcctggacgagctgtacgccgagt
ggtcggaggtcgtgtccacgaacttccgggacgcctccgggccggccatgaccgagatcggcga
gcagccgtgggggcgggagttcgccctgcgcgacccggccggcaactgcgtgcacttcgtggcc
gaggagcaggactgacacgtgctacgagatttcgattccaccgccgccttctatgaaaggttgg
gcttcggaatcgttttccgggacgccggctggatgatcctccagcgcggggatctcatgctgga
gttcttcgcccaccccaacttgtttattgcagcttataatggttacaaataaagcaatagcatc
acaaatttcacaaataaagcatttttttcactgcattctagttgtggtttgtccaaactcatca
atgtatcttatcatgtctgtataccgtcgacctctagctagagcttggcgtaatcatggtcata
gctgtttcctgtgtgaaattgttatccgctcacaattccacacaacatacgagccggaagcata
aagtgtaaagcctggggtgcctaatgagtgagctaactcacattaattgcgttgcgctcactgc
ccgctttccagtcgggaaacctgtcgtgccagctgcattaatgaatcggccaacgcgcggggag
aggcggtttgcgtattgggcgctcttccgcttcctcgctcactgactcgctgcgctcggtcgtt
cggctgcggcgagcggtatcagctcactcaaaggcggtaatacggttatccacagaatcagggg
ataacgcaggaaagaacatgtgagcaaaaggccagcaaaaggccaggaaccgtaaaaaggccgc
gttgctggcgtttttccataggctccgcccccctgacgagcatcacaaaaatcgacgctcaagt
cagaggtggcgaaacccgacaggactataaagataccaggcgtttccccctggaagctccctcg
tgcgctctcctgttccgaccctgccgcttaccggatacctgtccgcctttctcccttcgggaag
cgtggcgctttctcatagctcacgctgtaggtatctcagttcggtgtaggtcgttcgctccaag
ctgggctgtgtgcacgaaccccccgttcagcccgaccgctgcgccttatccggtaactatcgtc
ttgagtccaacccggtaagacacgacttatcgccactggcagcagccactggtaacaggattag
cagagcgaggtatgtaggcggtgctacagagttcttgaagtggtggcctaactacggctacact
agaagaacagtatttggtatctgcgctctgctgaagccagttaccttcggaaaaagagttggta
gctcttgatccggcaaacaaaccaccgctggtagcggtggtttttttgtttgcaagcagcagat
tacgcgcagaaaaaaaggatctcaagaagatcctttgatcttttctacggggtctgacgctcag
tggaacgaaaactcacgttaagggattttggtcatgagattatcaaaaaggatcttcacctaga
tccttttaaattaaaaatgaagttttaaatcaatctaaagtatatatgagtaaacttggtctga
cagttaccaatgcttaatcagtgaggcacctatctcagcgatctgtctatttcgttcatccata
gttgcctgactccccgtcgtgtagataactacgatacgggagggcttaccatctggccccagtg
ctgcaatgataccgcgagacccacgctcaccggctccagatttatcagcaataaaccagccagc
cggaagggccgagcgcagaagtggtcctgcaactttatccgcctccatccagtctattaattgt
tgccgggaagctagagtaagtagttcgccagttaatagtttgcgcaacgttgttgccattgcta
caggcatcgtggtgtcacgctcgtcgtttggtatggcttcattcagctccggttcccaacgatc
aaggcgagttacatgatcccccatgttgtgcaaaaaagcggttagctccttcggtcctccgatc
gttgtcagaagtaagttggccgcagtgttatcactcatggttatggcagcactgcataattctc
ttactgtcatgccatccgtaagatgcttttctgtgactggtgagtactcaaccaagtcattctg
agaatagtgtatgcggcgaccgagttgctcttgcccggcgtcaatacgggataataccgcgcca
catagcagaactttaaaagtgctcatcattggaaaacgttcttcggggcgaaaactctcaagga
tcttaccgctgttgagatccagttcgatgtaacccactcgtgcacccaactgatcttcagcatc
ttttactttcaccagcgtttctgggtgagcaaaaacaggaaggcaaaatgccgcaaaaaaggga
ataagggcgacacggaaatgttgaatactcatactcttcctttttcaatattattgaagcattt
atcagggttattgtctcatgagcggatacatatttgaatgtatttagaaaaataaacaaatagg
ggttccgcgcacatttccccgaaaagtgccacctgac

According to some embodiments, p138_Lenti_CBA_flex-Chronos-GA80s-GFP-WPRE_10-FP-CBA_sequencing result (801 bp) comprises SEQ ID NO: 87, shown below_.

NNNNNNNNNNNNNNNNNNNNNNNNNGTTCTGCCTTCTTCTTTTTCCTACA
GCTCCTGGGCAACGCCACCATGGCACCCAACTTTTCTATACAAAGTTGTA
TCTCTGTCTCGACAAGCCCAGTTTCTATTGGTCTCCTTAAACCTGTCTTG
TAACCTTGATACTTACCAGGTGGTGGCCCAGGAAGCCCCAGGTGTTTTTG
CTTATCAGATCCAGGATCAGATGGCCGATGCCGCTGGTGTATGGGGTGAT
CAGGCCGAGGCCCTCGTGTCCGGCAATGAACATCACGGGGAACATCAGCC
AGCTGCAGAAAAAGACGTAGGCCATGATTTTACAGATCTTTCTGCACACG
CCCTTAGGCAGTGTGTGGTAGCTTTCGATGTACACCTTGGCGATCTGAAA
GAAGCATGTGACGCCGTAAAAGAGTCCGATCATGAAGAACAGAATTTTCA
GAGGGCCCTTGGTAAAAGCGGCGGTGATTCCCCACACGATGTTGCCGATG
TCTGTCACGAGGATTGTCATGGTTCTCTTGCTGTACTCCTCGTGCAGTCC
AGTCAGGTTGCTCAGGTGGATCAGGATAACGGGGCAGGTCAGCAGCCACA
TGGAGTACCGCAGCCAGATCACGGCGCCGCCGTTGGTCTGATACACGGTG
GCAGGGCTGTCCACTTCGTGAAACAGCTCGATAAAGCACTTCACCAGCTC
AATCACACACACGTACACTTCCTCCCAGCCGGTTGTGGCCTTGNATGAGT
GCCANCCGTANNNATCAGCTGCACNATGGNCACNATCNCNGTGAACCNNT
G

According to some embodiments, p138_Lenti_CBA_flex-Chronos-GA80s-GFP-WPRE_10-RP-WPRE-01 (862 bp) comprises SEQ ID NO: 88, shown below.

NNNNNNNNNNNNNGNNNNANAGCAGCGTATCCACATAGCGTAAAAGGAGC
AACATAGTTAAGAATACCAGTCAATCTTTCACAAATTTTGTAATCCAGAG
GTTGATTTCACTTGTACAGCTCGTCCATGCCATGTGTGATCCCAGCAGCG
GTCACAAACTCCAGCAGGACCATGTGGTCTCTCTTTTCGTTGGGATCTTT
AGACAGGGCAGACTGGGTGGACAGGTAATGGTTGTCTGGGAGGAGCACAG
GGCCGTCGCCGATTGGAGTGTTCTGTTGATAATGGTCGGCCAGCTGCACG
GATCCATCCTCAATGTTGTGTCTGATCTTGAAGTTGACCTTGATGCCATT
CTTTTGCTTGTCGGCCATGATGTACACATTGTGGGAGTTATAGTTGTATT
CCAGCTTGTGGCCGAGAATGTTTCCATCCTCCTTAAAGTCAATGCCCTTC
AGCTCGATTCTATTCACCAGGGTGTCACCTTCGAACTTGACTTCAGCGCG
GGTCTTGTAGTTCCCGTCATCTTTGAAAAAGATGGTTCTCTCCTGCACAT
AGCCCTCGGGCATGGCGCTCTTGAAAAAGTCATGCTGCTTCATATGGTCT
GGGTATCTGGAAAAGCACTGCACGCCATAGGTCAGGGTAGTGACCAGTGT
TGGCCATGGCACAGGGAGCTTTCCAGTGGTGCAGATGAATTTCAGGGTGA
GCTTTCCGTATGTGGCATCACCTTCACCCTCTCCGCTGACANAAAATTTG
TGCCCATTCACATCGCCATCCAGTTCCNCGAGAATTGGGACACNCCAGTG
AACAGTTCCTCNCCTTGCTCTTGTCNTCGTCATTCNTATAATCGGAAGAN
GGNGGNGATGAN

miRNA Knockdown

Based on algorithms, a total of 80 miRNA constructs were designed to target the C9orf72 gene. A cell model-based screening will be performed to find the top candidates. The screening will be performed on stable cell model generated by p136_Lenti_CBA_tandomarray-Sense-GA80s-GFP-WPRE or p137_Lenti_CBA_tandomarray-AntiSense-GA80s-GFP-WPRE

Experiments will be performed using cells transfected with:
(1) p136_Lenti_CBA_tandomarray-Sense-GA80s-GFP-WPRE;
(2) p137_Lenti_CBA_tandomarray-AntiSense-GA80s-GFP-WPRE or
(3) p138_Lenti_CBA_flex-Chronos-GA80s-GFP-WPRE. Untransfected cells served as control. One day after transfection, cells will be infected with virus carrying the top miRNA constructs. At day 3, cell will be stained with anti-GFP antibody and GFP fluorescence will be detected to determine c9orf72 knockdown. This experiment will be used to demonstrate the efficiency of miRNA knockdown.

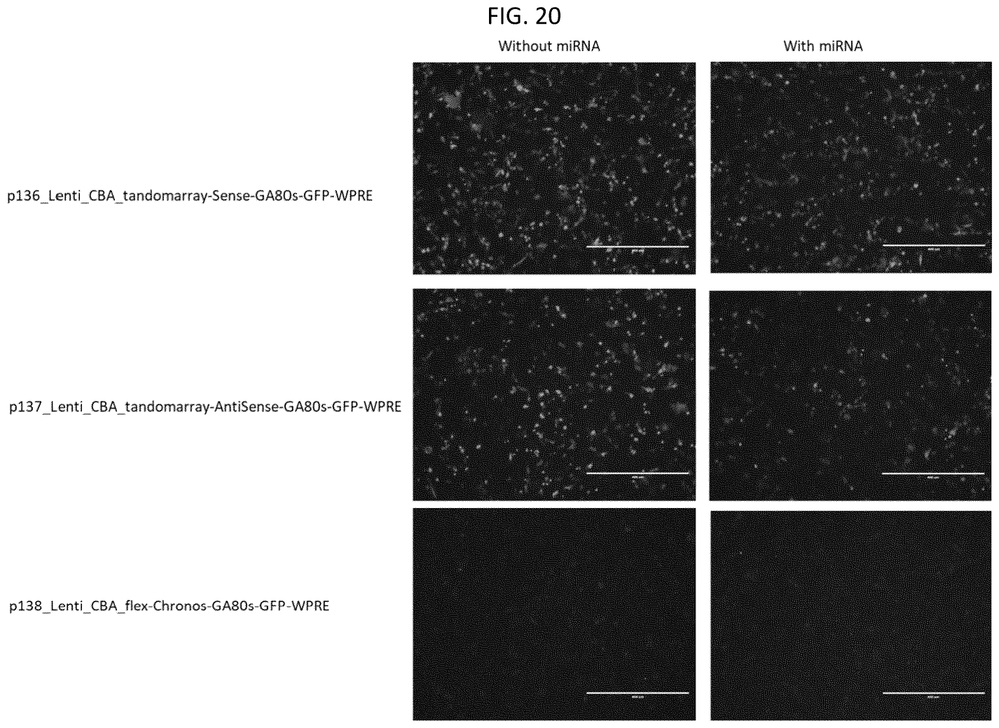

FIG. 20 shows the results of another set of experiments, which demonstrated that using p136_Lenti_CBA_tandomarray-Sense-GA80s-GFP-WPRE or p137_Lenti_CBA_tandomarray-AntiSense-GA80s-GFP-WPRE, a fluorescence reporter system can be built that can be used to evaluate the efficiency of miRNA knockdown.

Puro & BSD Positive Selection for 3, 6, 9, 12 Days.

Puro+ selection will be effective from 24 hrs. BSD+ selection will take longer, which is advantageous for quantifying protein knock-down turnover.

Samples will be collected at 3, 6, 9, 12, 15 days for quantification.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

### REFERENCES

Angela Schoolmeesters, M. L. K., Annaleen Vermeulen, Anja Smith, *Mayya Shveygert, *Xin Zhou, *Robert Blelloch (2017). “Smart-Lenti-miRNA-Vector” Keystone Pposter.
Barta, T., et al. (2016). “miRNAsong: a web-based tool for generation and testing of miRNA sponge constructs in silico.” Sci Rep 6: 36625.
Bofill-De Ros, X. and S. Gu (2016). “Guidelines for the optimal design of miRNA-based shRNAs.” Methods 103: 157-166.
Bofill-De Ros, X., et al. (2019). “Structural Differences between Pri-miRNA Paralogs Promote Alternative Drosha Cleavage and Expand Target Repertoires.” Cell Rep 26(2): 447-459 e444.
Bofill-De Ros, X., et al. (2019). “51-Structural Differences between Pri-miRNA Paralogs Promote Alternative Drosha Cleavage and Expand Target Repertoires.”
Chen, Z., et al. (2006). “Modeling CTLA4-linked autoimmunity with RNA interference in mice.” Proc Natl Acad Sci USA 103(44): 16400-16405.
DeJesus-Hernandez, M., et al. (2011). “Suppl. Infor. Expanded GGGGCC hexanucleotide repeat in noncoding region of C9ORF72 causes chromosome 9p-linked FTD and ALS.” Neuron.
DeJesus-Hernandez, M., et al. (2011). “Expanded GGGGCC hexanucleotide repeat in noncoding region of C9ORF72 causes chromosome 9p-linked FTD and ALS.” Neuron 72(2): 245-256.
Dow, L. E., et al. (2012). “Suppl. Infor. A pipeline for the generation of shRNA transgenic mice.” Nat Protoc.
Dow, L. E., et al. (2012). “A pipeline for the generation of shRNA transgenic mice.” Nat Protoc 7(2): 374-393.
Farg, M. A., et al. (2014). “C9ORF72, implicated in amytrophic lateral sclerosis and frontotemporal dementia, regulates endosomal trafficking.” Hum Mol Genet 23(13): 3579-3595.
Fellmann, C, et al. (2013). “Suppl. Infor. An optimized microRNA backbone for effective single-copy RNAi.” Cell Rep.
Fellmann, C, et al. (2013). “An optimized microRNA backbone for effective single-copy RNAi.” Cell Rep 5(6): 1704-1713.
Hauser, F., et al. (2013). “A genomic-scale artificial microRNA library as a tool to investigate the functionally redundant gene space in Arabidopsis.” Plant Cell 25(8): 2848-2863.
Hu, J. et al., J., et al. (2015). “Engineering Duplex RNAs for Challenging Targets: Recognition of GGGGCC/CCCCGG Repeats at the ALS/FTD C9orf72 Locus.” Chem Biol 22(11): 1505-1511.
Jiang, J., et al. (2016). “Gain of Toxicity from ALS/FTD-Linked Repeat Expansions in C9ORF72 Is Alleviated by Antisense Oligonucleotides Targeting GGGGCC-Containing RNAs.” Neuron 90(3): 535-550.
Jiang, L., et al. (2017). “NEAT1 scaffolds RNA-binding proteins and the Microprocessor to globally enhance pri-miRNA processing.” Nat Struct Mol Biol 24(10): 816-824.
Martier, R., et al. (2019). “Targeting RNA-Mediated Toxicity in C9orf72 ALS and/or FTD by RNAi-Based Gene Therapy.” Mol Ther Nucleic Acids 16: 26-37.
Martier, R., et al. (2019). “Suppl. Infor. Artificial MicroRNAs Targeting C9orf72 Can Reduce Accumulation of Intra-nuclear Transcripts in ALS and FTD Patients.” Mol Ther Nucleic Acids.
Martier, R., et al. (2019). “Artificial MicroRNAs Targeting C9orf72 Can Reduce Accumulation of Intra-nuclear Transcripts in ALS and FTD Patients.” Mol Ther Nucleic Acids 14: 593-608.
Miniarikova, J., et al. (2016). “Design, Characterization, and Lead Selection of Therapeutic miRNAs Targeting
Huntingtin for Development of Gene Therapy for Huntington's Disease.” Mol Ther Nucleic Acids 5: e297.
Riba, A., et al. (2017). “Explicit Modeling of siRNA-Dependent On- and Off-Target Repression Improves the Interpretation of Screening Results.” Cell Syst 4(2): 182-193 e184.
Urbanek-Trzeciak, M. O., et al. (2018). “miRNAmotif-A Tool for the Prediction of Pre-miRNA(−)Protein Interactions.” Int J Mol Sci 19(12).
Urbanek-Trzeciak, M. O., et al. (2018). “Supplementary Information miRNAmotif-A Tool for the Prediction of Pre-miRNA(−)Protein Interactions.” Int J Mol Sci.
Watanabe, C., et al. (2016). “S1-Quantitative evaluation of first, second, and third generation hairpin systems reveals the limit of mammalian vector-based RNAi.” RNA Biol.
Watanabe, C., et al. (2016). “Quantitative evaluation of first, second, and third generation hairpin systems reveals the limit of mammalian vector-based RNAi.” RNA Biol 13(1): 25-33.
Watanabe, C., et al. (2016). “S2-Quantitative evaluation of first, second, and third generation hairpin systems reveals the limit of mammalian vector-based RNAi.” RNA Biol.
Watanabe, C., et al. (2016). “S3-Quantitative evaluation of first, second, and third generation hairpin systems reveals the limit of mammalian vector-based RNAi.” RNA Biol.
Zhang, X., et al. (2016). “Cell-free 3D scaffold with two-stage delivery of miRNA-26a to regenerate critical-sized bone defects.” Nat Commun 7: 10376.

1. A nucleic acid sequence encoding a C9ORF72 protein, wherein the nucleic acid sequence is codon optimized.

2. The nucleic acid sequence of claim 1, wherein the codon optimized sequence is selected from a sequence set forth in Table 2.

3. The nucleic acid sequence of claim 1, comprising a nucleic acid sequence that is at least 85% identical to a nucleic acid sequence selected from any one of SEQ ID NOs 21-52 and 100-106.

4. A transgene expression cassette comprising
a promoter; and
the nucleic acid sequence of claim 1.

5. The transgene expression cassette of claim 4, further comprising:
a c9orf72 sense transcript specific inhibitor; and
a c9orf72 antisense transcript specific inhibitor.

6. The transgene expression cassette of claim 5, wherein the c9orf72 sense transcript specific inhibitor is selected from the group consisting of: a nucleic acid, an aptamer, an antibody, a peptide, or a small molecule.

7. (canceled)

8. (canceled)

9. The transgene expression cassette of claim 5, wherein:
the sense transcript inhibitor is selected from an miRNA set forth in Table 4;
the antisense transcript inhibitor is selected from an miRNA set forth in Table 3.

10.-12. (canceled)

13. The transgene expression cassette of claim 4, wherein the promoter is specific for expression in neurons.

14. (canceled)

15. (canceled)

16. A nucleic acid vector comprising the expression cassette of claim 4.

17. The vector of claim 16, wherein the vector is an adeno-associated viral (AAV) vector.

18. The vector of claim 17, wherein the serotype of the AAV vector is derived from an AAV serotype selected from the group consisting of AAV1, AAV2, AAV3, AAV4, AAV5, AAV6, AAV7, AAV8, AAV9, AAV10, AAV11, and AAV12.

19. (canceled)

20. A mammalian cell comprising the vector of claim 16.

21.-25. (canceled)

26. A method of treating or preventing the progression of a c9orf72 associated disease, comprising administering to a subject in need thereof the vector of claim 16, thereby treating the c9orf72 associated disease in the subject.

27. (canceled)

28. The method of claim 26, wherein:
the c9orf72 associated disease is a c9orf72 hexanucleotide repeat expansion associated disease;
the c9orf72 associated disease is a neurodegenerative disease;
the subject has one or more mutations in the c9orf72 gene; and/or
the expression of c9orf72 is inhibited or suppressed.

29. (canceled)

30. The method of claim 28, wherein the neurodegenerative disease is selected from the group consisting of: amyotrophic lateral sclerosis (ALS), frontotemporal dementia (FTD), Parkinson disease, progressive supranuclear palsy, ataxia, corticobasal syndrome, Huntington disease-like syndrome, Creutzfeldt-Jakob disease and Alzheimer disease.

31.-37. (canceled)

38. A method for inhibiting the expression of c9orf72 gene in a cell wherein the c9orf72 gene comprises a hexanucleotide repeat expansion, comprising administering the cell a composition comprising the vector of claim 16.

39. The method of claim 38, wherein the hexanucleotide repeat expansion causes loss of function of C9ORF72 protein and/or toxic gain of function from sense and antisense c9orf72 repeat RNA or from dipeptide repeats.

40. The method of claim 38, wherein the cell is a mammalian cell.

41. (canceled)

42. The method of claim 26, wherein the vector is administered by intracranial administration.

43. (canceled)

44. A kit comprising the vector of claim 16 and instructions for use.

45. (canceled)
